(19) United States
(12) Patent Application Publication
(10) Pub. No.: US 2026/0364080 A1
(43) Pub. Date: Nov. 19, 2026

(54) PYRAZOLO-PYRIMIDINONE COMPOUNDS FOR USE IN METHODS OF INHIBITING WEE1 A KINASE

(71) Applicant: Acrivon Therapeutics, Inc., Watertown, MA (US)

(72) Inventors: Hans Mattias Jönsson, Lund (SE); Nima Rajabi, Lund (SE); Filip Alexander Paulsen, Malmö (SE); Andrea Öhlander, Malmö (SE); Björn Borgström, Löddeköpinge (SE); Magnus Munck af Rosenschöld, Lund (SE); Lars Thomas Brimert, Blentarp (SE); Caroline Maria Wigerup, Dalby (SE); John Henry van Duzer, Concord, MA (US); David Proia, Newton, MA (US); Lei Shi, Arlington, MA (US); Åsa Helén Nilsson, Åkarp (SE); Mette Laursen, Limhamn (SE); Bo Roger Svensson, Lund (SE); Joon O. Jung, Newton, MA (US)

(21) Appl. No.: 19/491,155

(22) PCT Filed: Jun. 7, 2024
(86) PCT No.: PCT/US2024/033065

§ 371 Date: Dec. 8, 2025

Related U.S. Application Data
(60) Provisional application No. 63/472,105, filed on Jun. 9, 2023.
(60) Provisional application No. 63/608,086, filed on Dec. 8, 2023.
(60) Provisional application No. 63/635,197, filed on Apr. 17, 2024.

(51) Int. Cl. A61K 31/519; A61P 35/00; C07B 59/00; C07D 487/04; C07D 519/00

(52) U.S. Cl. CPC A61K 31/519; A61P 35/00; C07B 59/002; C07D 487/04; C07D 519/00

(57) ABSTRACT

Pyrazolo-pyrimidinone compounds of formula I: for use in methods of inhibiting Wee1A kinase or both Wee1A kinase and Myt1 kinase.

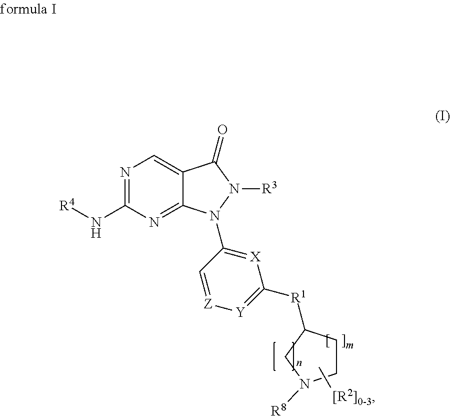

### BACKGROUND

Cells are continuously challenged with endogenous and exogenous agents that influence DNA integrity. To maintain genomic stability and prevent unwanted propagation of damaged DNA, cells have established an organized signaling network that recognizes DNA lesions and halts the cell cycle to allow the DNA to be correctly repaired before resuming DNA replication or cell division. The DNA damage response and the cell cycle are tightly linked via several cell cycle checkpoints that are important control steps for maintaining genomic integrity.

Cancer cells frequently have a defective G1/S checkpoint, often via disrupted p53 activity due to mutations or deletion, or inactivation by viral oncoproteins. Therefore, cancer cells rely heavily on other cell cycle checkpoints, including the G2/M checkpoint, to avoid accumulation of deleterious DNA damage and mitotic catastrophe. As such, cancer cells are hypothesized to be particularly vulnerable to inhibition of proteins that safeguard the entry into mitosis. Matheson, C. J. et al Trends Pharmacol Sci 37, 872-881 (2016).

Wee1A kinase is a tyrosine kinase belonging to the Wee1 kinase family, including Wee1A kinase, Wee1B kinase, and Myt1 kinase. Rorà, A. G. L. et al J Hematol Oncol 13, 126 (2020). The primary role for this kinase family is to regulate cell cycle progression and entry into mitosis (Wee1A kinase and Myt1 kinase) or meiosis (Wee1B kinase). The key complex regulating mitotic entry is Cdk1/cyclin B1 complex, also known as the mitosis-promoting factor. Wee1A kinase constrains Cdk1/cyclin B1 complex activity by phosphorylating Cdk1 on the inhibitory tyrosine 15 site (Y¹⁵). Hence, inhibition of Wee1A kinase effectively promotes Cdk1/cyclin B1 complex activity by preventing inhibitory Y¹⁵ phosphorylation. Untimely activation of Cdk1/cyclin B complex promotes premature entry into mitosis with unresolved DNA damages, ultimately leading to mitotic catastrophe and cell death.

In addition to its well-established role in regulating mitotic entry at the G2/M checkpoint, Wee1A kinase has also been suggested to be important in the intra-S checkpoint by limiting activity of Cdk2. Elbæk, C. R. et al Cell Reports 38, 110261 (2022); Elbæk, C. R. et al Mutat Res Fundam Mol Mech Mutagen 819-820, 111694 (2020). The activity of Cdk2 is regulated by Wee1A kinase in the same way as Cdk1 by tyrosine 15 phosphorylation. Cdk2 is the primary Cdk driving DNA replication and inhibition of Wee1A kinase leads to excessive DNA replication, leading to exhaustion of nucleotide pools and degradation of the ribonucleotide reductase subunit RRM2 (Pfister, S. X. et al Cancer Cell 28, 557-568 (2015)). Pfister et al. showed that Wee1A kinase inhibition selectively kills H₃K₃₆me₃-deficient cancer cells through dNTP starvation resulting from RRM2 depletion. The histone methyl transferase SETD2 catalyzes H₃K₃₆me₃, which promotes RRM2 expression and synthesis of dNTPs. Inactivation of SETD2 gene is frequent in clear cell renal carcinomas (ccRCC) and might therefore be sensitive to Wee1A kinase inhibition. A phase II trial is testing AZD1775 in SETD2-deficient solid tumors (NCT03284385).

Wee1A kinase has also been suggested to have a role in controlling histone stoichiometry by phosphorylation of core histone H₂B at tyrosine 37 at late S phase. Koh, S.-B. Cell Signal 94, 110310 (2022).

Cancers associated with high-risk human papilloma virus (HPV) such as head and neck squamous cell carcinoma (HNSCC) showed increased sensitivity to Wee1A kinase inhibition. Diab, A. et al Proc National Acad Sci 117, 28287-28296 (2020).

Several Wee1A kinase inhibitors are currently being tested in clinical trials (Bukhari, A. B. et al Frontiers Oncol 12, 828684 (2022) and have shown activity in many indications. A phase II study of the Wee1A kinase inhibitor AZD1775 (adavosertib) has shown promising results in women with uterine serous carcinoma. Liu, J. F. et al J Clin Oncol 39, 1531-1539 (2021). Adavosertib has also shown effect compared to active monitoring in RAS/TP₅₃ mutated metastatic colorectal cancer. Seligmann, J. F. et al J Clin Oncol 39, 3705-3715 (2021). In a phase 1b trial of 18 patients with platinum-resistant ovarian cancer, the combination of ZN-c3 (azenosertib) plus paclitaxel led to an ORR of 50% (J Clin Oncol 41, 2023 (suppl 16; abstr 5513)). Given the encouraging signs of clinical activity with Wee1A kinase inhibition, there is an urgent need for novel Wee1A kinase inhibitors with improved potency and selectivity, as well as for compounds that inhibit both Wee1A kinase and Myt1 kinase to maximize the efficacy potential of this target class.

### SUMMARY

In some embodiments, the present disclosure provides a compound of formula I:

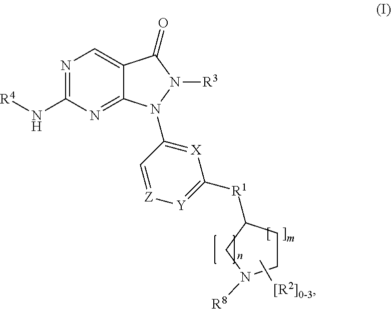

or a pharmaceutically acceptable salt thereof, wherein each of X, Y, Z, R¹, R², R³, R⁴, R⁸, n and m are as defined below and described herein.

In some embodiments, the present disclosure provides a pharmaceutical composition comprising a compound of formula I, or a pharmaceutically acceptable salt thereof, and a pharmaceutically acceptable carrier.

In some embodiments, the present disclosure provides a method of inhibiting Wee1A kinase in a patient or in a biological sample, the method comprising administering to the patient or contacting the biological sample with a compound of formula I, or a pharmaceutically acceptable salt thereof.

In some embodiments, the present disclosure provides a method of inhibiting both Wee1A kinase and Myt1 kinase in a patient or in a biological sample, the method comprising administering to the patient or contacting the biological sample with a compound of formula I, or a pharmaceutically acceptable salt thereof.

In some embodiments, the present disclosure provides a method of treating a disease or disorder associated with Wee1A kinase, the method comprising administering to a patient in need thereof a compound of formula I, or a pharmaceutically acceptable salt thereof.

In some embodiments, the present disclosure provides a method of treating a disease or disorder associated with both Wee1A kinase and Myt1 kinase, the method comprising administering to a patient in need thereof a compound of formula I that has dual activity, or a pharmaceutically acceptable salt thereof.

In some such embodiments, the disease or disorder associated with Wee1A kinase is a cancer. In some such embodiments, the disease or disorder associated with Wee1A kinase and Myt1 kinase is a cancer. In some embodiments, the cancer is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

### DETAILED DESCRIPTION

### 1. General Description of Compounds of the Disclosure

In some embodiments, the present disclosure provides inhibitors of Wee1A kinase. In some embodiments, such compounds include those of the formulae described herein, or a pharmaceutically acceptable salt thereof, wherein each variable is as defined and described herein.

In some embodiments, the present disclosure provides a compound having structural formula AA:

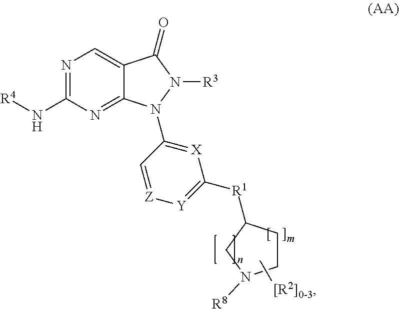

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged or fused C₃-C₅ cycloalkyl ring, or one R² and R⁸ are optionally taken together to form a bridged or fused 3-5 membered ring;
R³ is —C₁-C₃ alkyl or —CH₂—CH═CH₂;

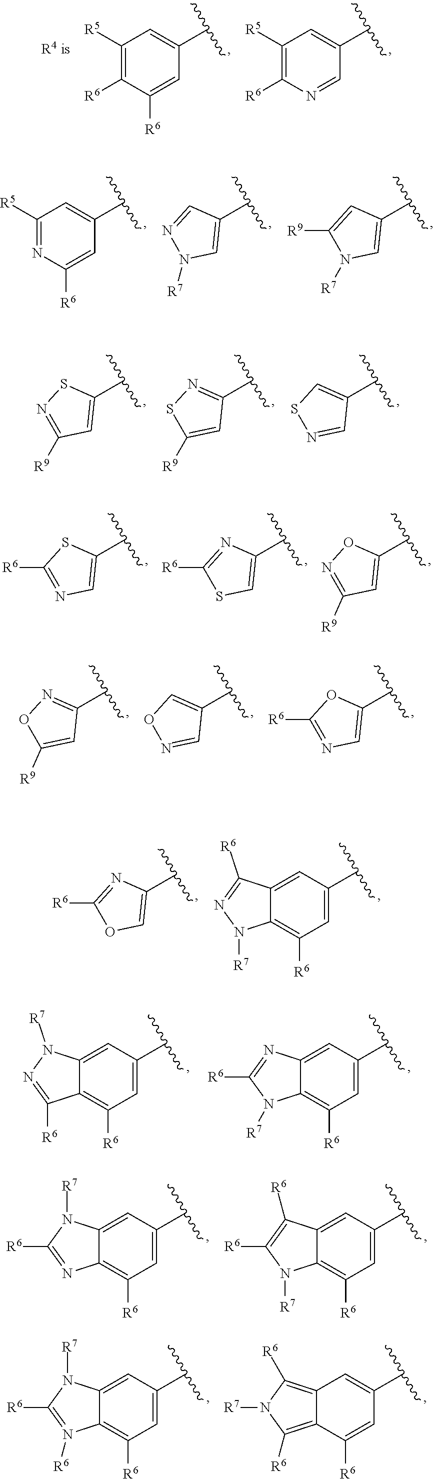

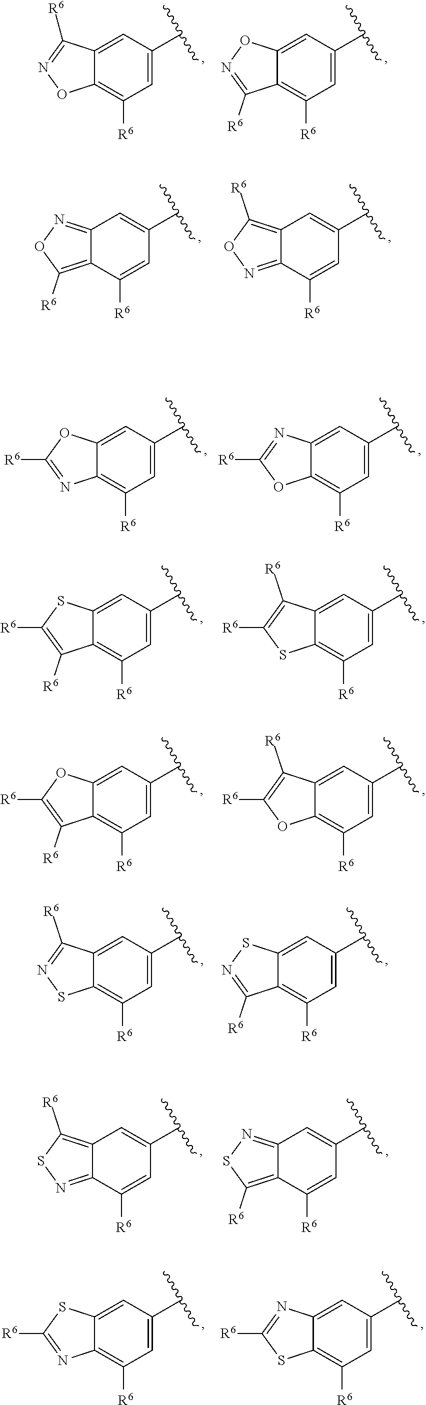

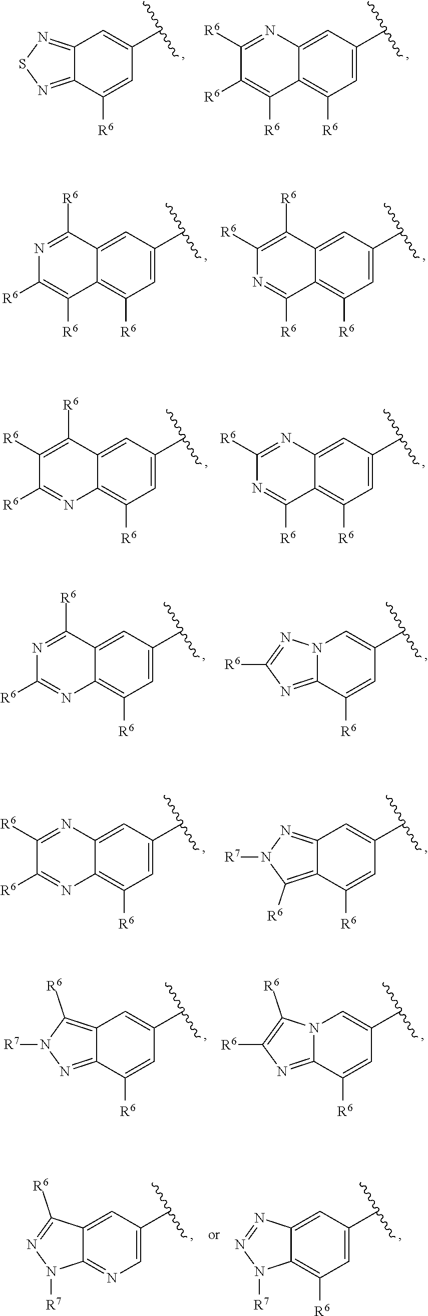

wherein “ [symbol] ” represents a point of attachment of R⁴ to the compound;
each R⁵ and each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl, wherein any C₁-C₄ alkyl or C₁-C₄ alkylene portion of R⁷ is optionally substituted with one or more substituents independently selected from halo and —CN;
R⁸ is hydrogen, —C₁-C₄ alkyl, or a 4-6 membered saturated heterocycle;
R⁹ is hydrogen, halo, —CN, —C₁-C₄ alkyl optionally substituted with halo, —O—C₁-C₄ alkyl optionally substituted with halo, or an optionally substituted phenyl;
m is 0, 1, 2, 3 or 4;
n is 0, 1, 2 or 3; and
m+n is 1, 2, 3, or 4,
   wherein any hydrogen atom is optionally replaced with deuterium.

In some embodiments, the present disclosure provides a compound having structural formula A:

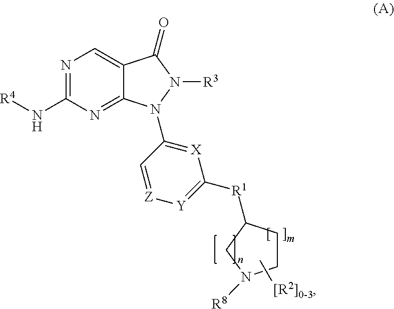

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged C₃-C₅ cycloalkyl ring, or one R² and R⁸ are optionally taken together to form a bridged 3-5 membered ring;
R³ is —C₁-C₃ alkyl or —CH₂—CH═CH₂;

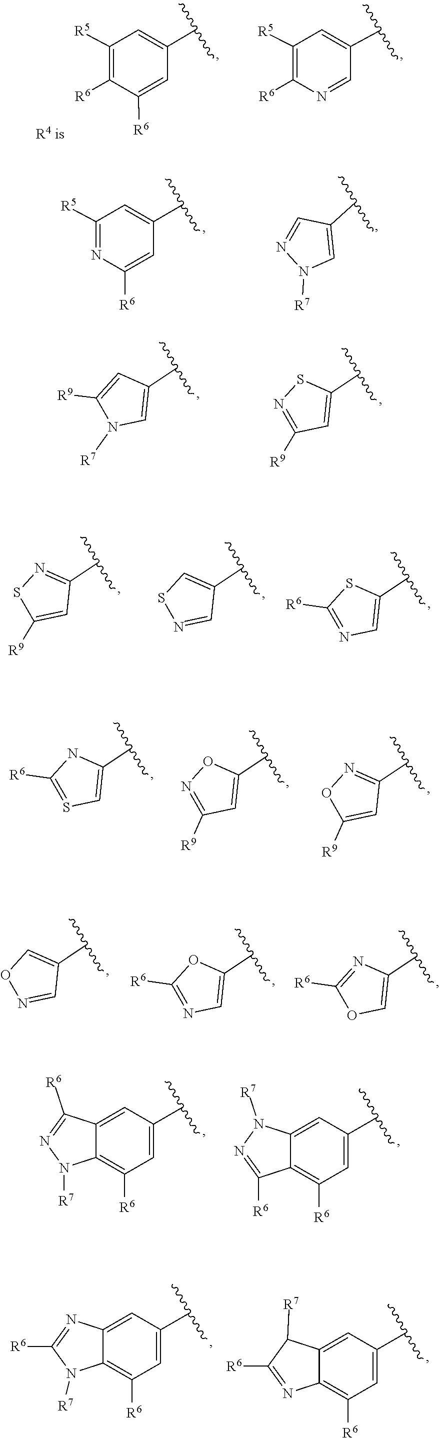

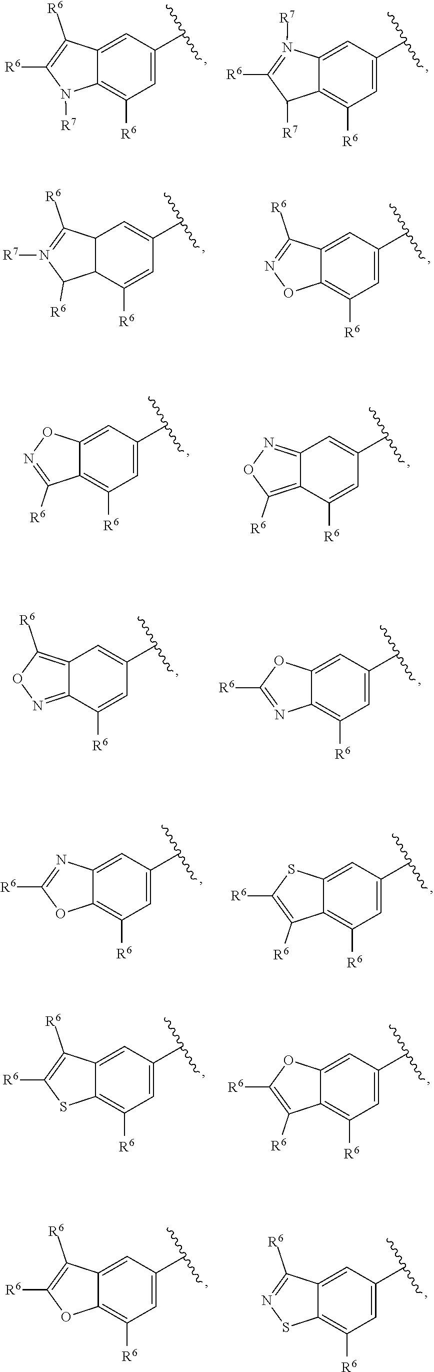

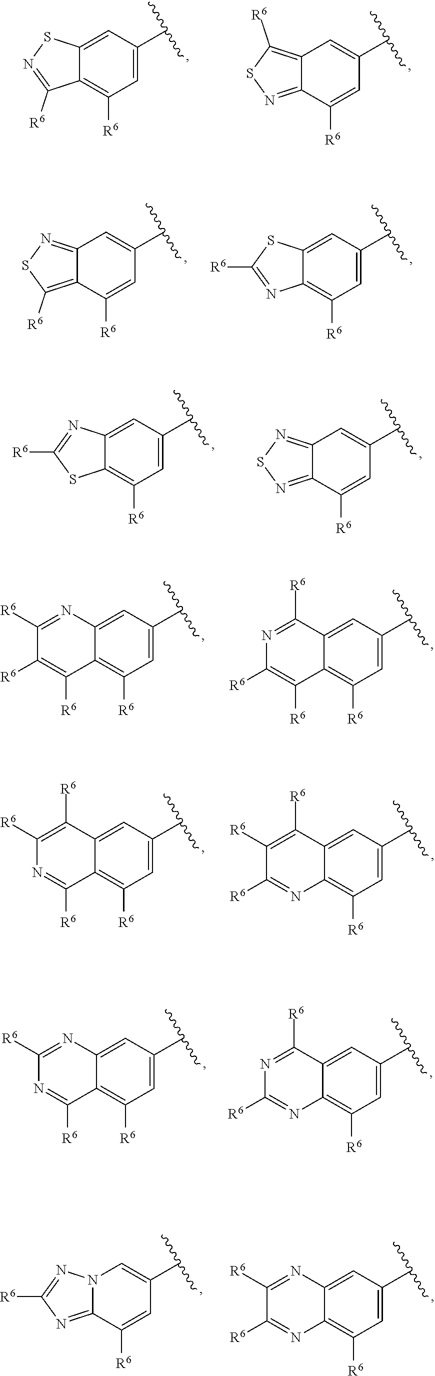

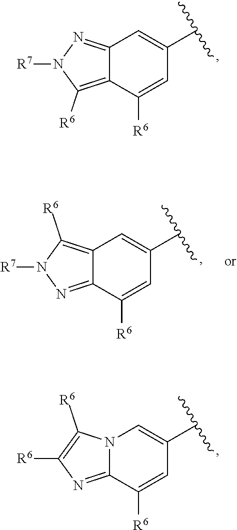

wherein “ [symbol] ” represents a point of attachment of R⁴ to the compound;
each R⁵ and each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl, wherein any C₁-C₄ alkyl or C₁-C₄ alkylene portion of R⁷ is optionally substituted with one or more substituents independently selected from halo and —CN;
R⁸ is hydrogen, —C₁-C₄ alkyl, or a 4-6 membered saturated heterocycle;
R⁹ is hydrogen, halo, —CN, —C₁-C₄ alkyl optionally substituted with halo, —O—C₁-C₄ alkyl optionally substituted with halo, or an optionally substituted phenyl;
m is 0, 1, 2, 3 or 4;
n is 0, 1, 2 or 3; and
m+n is 1, 2, 3, or 4,
   wherein any hydrogen atom is optionally replaced with deuterium.

In some embodiments, the present disclosure provides a compound having structural formula I:

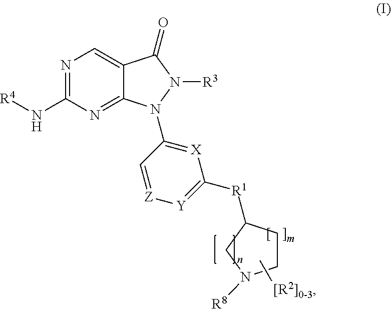

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged C₃-C₅ cycloalkyl ring, or one R² and R⁸ are optionally taken together to form a bridged 3-5 membered ring;
R³ is —C₁-C₃ alkyl or —CH₂—CH═CH₂;
R⁴ is

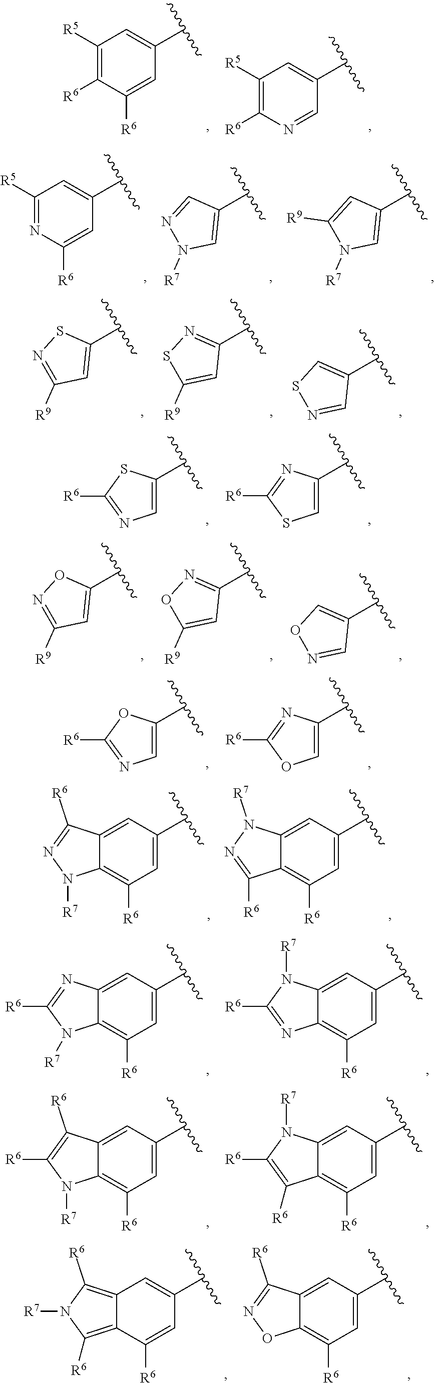

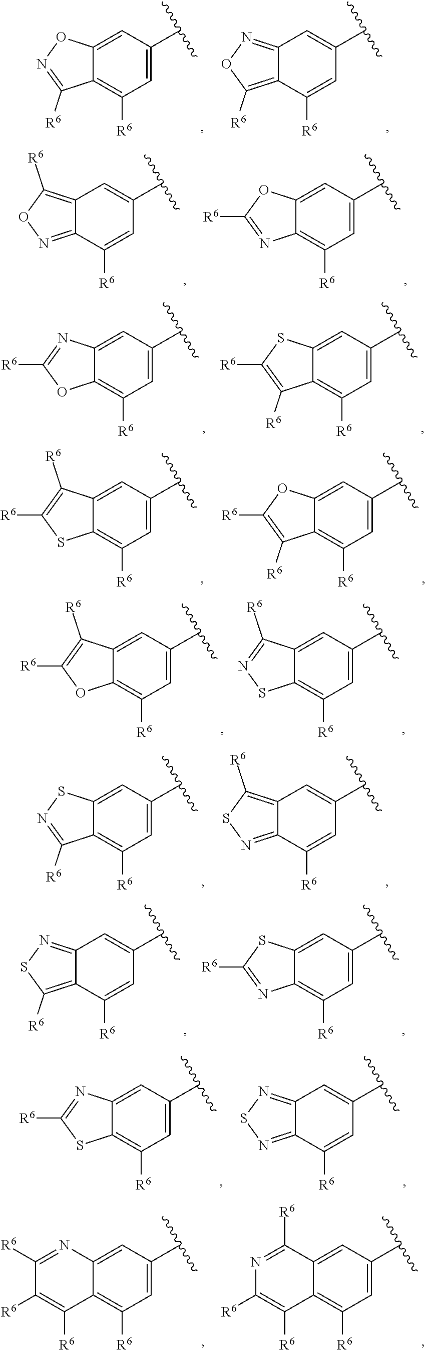

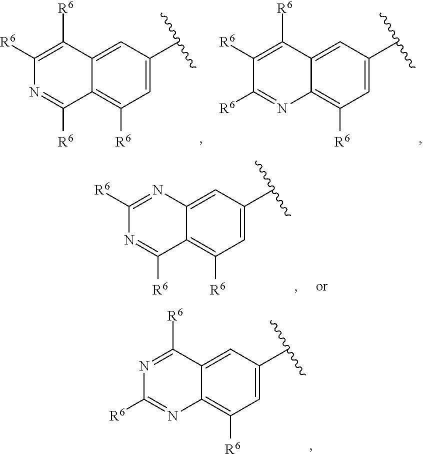

wherein “ [symbol] ” represents a point of attachment of R⁴ to the compound;
each R⁵ and each R⁶ is independently hydrogen, halo, —CN, —C₁-C₄ alkyl optionally substituted with halo, or —O—(C₁-C₄ alkyl) optionally substituted with halo, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, or —C₁-C₄ alkylene-O—C₁-C₄ alkyl, wherein any C₁-C₄ alkyl or C₁-C₄ alkylene portion of R⁷ is optionally substituted with one or more substituents independently selected from halo and —CN;
R⁸ is hydrogen or —C₁-C₄ alkyl;
R⁹ is hydrogen, halo, —CN, —C₁-C₄ alkyl optionally substituted with halo, —O—C₁-C₄ alkyl optionally substituted with halo, or an optionally substituted phenyl;
m is 0 or 1;
n is 0, 1, 2 or 3; and
m and n are not simultaneously 0.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocyclyl” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, as used herein, means a straight or branched hydrocarbon chain that is completely saturated. Exemplary alkyl groups include methyl, ethyl, n-propyl, isopropyl, n-butyl, sec-butyl, iso-butyl, tert-butyl, etc.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated” refers to a moiety that includes at least one double or triple bond. The term “partially unsaturated” is intended to encompass moieties having multiple sites of unsaturation, but when used to describe a ring, is not intended to include aryl or heteroaryl moieties, as herein defined.

The term “saturated” refers to a moiety that has no double or triple bonds.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a carbon atom.

The term “alkenylene” refers to a bivalent alkenyl group. A substituted alkenylene chain is a polymethylene group containing at least one double bond in which one or more hydrogen atoms are replaced with a substituent. Suitable substituents include those described below for a carbon atom.

The terms “halogen” and “halo” are used interchangeably and mean F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein each ring atom is carbon, at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic carbocyclic rings.

The term “cycloalkyl ring” refers to a fully saturated carbocyclic ring.

A first ring is “spiro-fused” to a second ring when the two rings share a single ring carbon atom. An example of a spiro-fused ring system is:

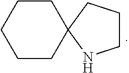

A first ring is “fused” to a second ring when the two rings share two ring carbon atoms that are directly connected to one another. Example of fused ring systems are:

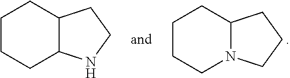 [SMILES: C1CCC2NCCC2C1 ; C1CCN2CCCC2C1]

A first ring is “bridged” to a second ring when the two rings share two ring carbon atoms that are separated by at least one ring carbon atom. Example of bridged ring systems are:

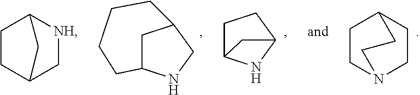

A reference to the number of atoms in a bridged ring, e.g., one R² and R⁸ are taken together to form a bridged 3-5 membered ring, refers to the two bridgehead ring atoms plus any additional ring atoms in between those bridgehead atoms that form the ring that is bridged to the existing ring. For example if the ring defined by the structure

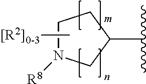

is

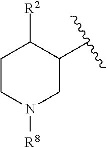

and R² and R⁸ are taken together to form

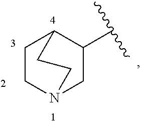 [SMILES: *C1CN2CCC1CC2]

the number of atoms in the bridged ring is 4 as enumerated in the bridged structure.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridinyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl or heteroaryl rings such that the resulting bi- or multicyclic ring system as a whole is fully aromatic. Nonlimiting examples include indolyl, isoindolyl, benzothienyl, benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, and phenoxazinyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolidinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The terms “cyano-substituted Cₓ-C_{y} alkyl” and “fluoro-substituted Cₓ-C_{y} alkyl” where each of x and y is an integer, refer to the corresponding alkyl having one or more of the indicated substituents in place of hydrogen.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; (CH₂)₀₋₄N(R^{◯}₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; N(R^{◯})C(S)NR^{◯}₂; —(CH₂)₀₋₄N(R^{◯})C(O)OR^{◯}; —N(R^{◯})N(R^{◯})C(O)R^{◯}; N(R^{◯})N(R^{◯})C(O)NR^{◯}₂; N(R^{◯})N(R^{◯})C(O)OR^{◯}; —(CH₂)₀₋₄C(O)R^{◯}₂; —C(S)R^{◯}; —(CH₂)₀₋₄C(O)OR^{◯}; —(CH₂)₀₋₄C(O)SR^{◯}; (CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}; —(CH₂)₀₋₄C(O)NR^{◯}₂; —C(S)NR^{◯}₂; —C(S)SR^{◯}; —SC(S)SR^{◯}, (CH₂)₀₋₄OC(O)NR^{◯}₂; C(O)N(OR^{◯})R^{◯}; —C(O)C(O)R^{◯}; —C(O)CH₂C(O)R^{◯}; —C(NOR^{◯})R^{◯}; (CH₂)₀₋₄SSR^{◯}; —(CH₂)₀₋₄S(O)₂R^{◯}; —(CH₂)₀₋₄S(O)₂OR^{◯}; (CH₂)₀₋₄OS(O)₂R^{◯}; —S(O)₂NR^{◯}₂; (CH₂)₀₋₄S(O)R^{◯}; N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; P(O)R^{◯}₂; OP(O)R^{◯}₂; —OP(O)(OR^{◯})₂; SiR^{◯}₃; —(C₁₋₄ straight or branched alkylene)O—N(R^{◯)}₂; or —(C₁₋₄ straight or branched alkylene)C(O)O—N(R^{◯)}₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, C(O)SR^{●}, —(C₁-4 straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-7-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, (haloR^{●}), OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or a substituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group and the substituted 5-6-membered saturated, partially unsaturated, or aryl ring having 0-4 heteroatoms independently selected from nitrogen, oxygen, or sulfur of R^{†} are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{●}₂, or NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)=₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, rotational isomers (atropisomers) and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and hair, skin, blood, saliva, urine, feces, semen, tears, or other body fluids or extracts thereof. Inhibition of activity of a protein kinase, for example, Wee1A kinase or a mutant thereof, in a biological sample is useful for a variety of purposes that are known to one of skill in the art. Examples of such purposes include, but are not limited to, blood transfusion, organ transplantation, biological specimen storage, and biological assays.

As used herein, a “disease or disorder associated with Wee1A kinase” or, alternatively, “a Wee1A kinase-mediated disease or disorder” means any disease or other deleterious condition in which Wee1A kinase, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds of the present disclosure that may be combined with the carrier materials to produce a composition in a single dosage form will vary depending upon the host treated, the particular mode of administration, etc. Preferably, provided compositions are formulated so that a dosage of between 0.01 to about 100 mg/kg, or about 0.1 mg/kg to about 50 mg/kg, and preferably from about 1 mg/kg to about 25 mg/kg, of subject body weight/day of the inhibitor can be administered to a patient receiving these compositions to obtain the desired therapeutic effect. The amount of a compound of the present disclosure in the composition will also depend upon the particular compound in the composition.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

As used herein, the term “inhibitor” is defined as a compound that binds to and/or inhibits the target protein kinase with measurable affinity. In certain embodiments, an inhibitor has an IC₅₀ and/or binding constant of less than about 50 μM, less than about 1 μM, less than about 500 nM, less than about 100 nM, less than about 50 nM, less than about 20 nM, or less than about 10 nM.

The terms “measurable affinity” and “measurably inhibit,” as used herein, means a measurable change in Wee1A kinase or Myt1 kinase activity between a sample comprising a compound of the present disclosure, or composition thereof, and an equivalent sample comprising Wee1A kinase or Myt1 kinase, in the absence of said compound, or composition thereof.

As used herein the terms “dual inhibitor” and “dual Wee1A kinase/Myt1 kinase inhibitor” are used interchangeably and mean a compound disclosed herein that meets one or more of the following criteria: 1) a Myt1 kinase binding activity IC₅₀ of ≤100 nM (i.e., “A” or “B” rated in Table 3); or 2) a Myt1 kinase target engagement EC₅₀ of ≤500 nM (i.e., “A” or “B” rated in Table 4).

### 3. Description of Exemplary Compounds

In some embodiments, the present disclosure provides a compound having structural formula AA:

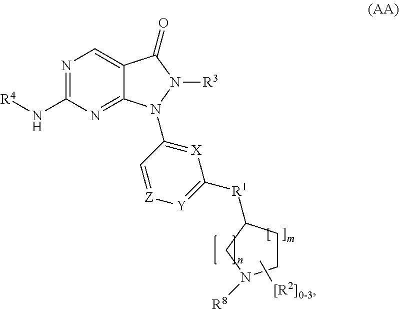

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged or fused C₃-C₅ cycloalkyl ring, or one R² and R⁸ are optionally taken together to form a bridged or fused 3-5 membered ring;
R³ is —C₁-C₃ alkyl or —CH₂—CH═CH₂;

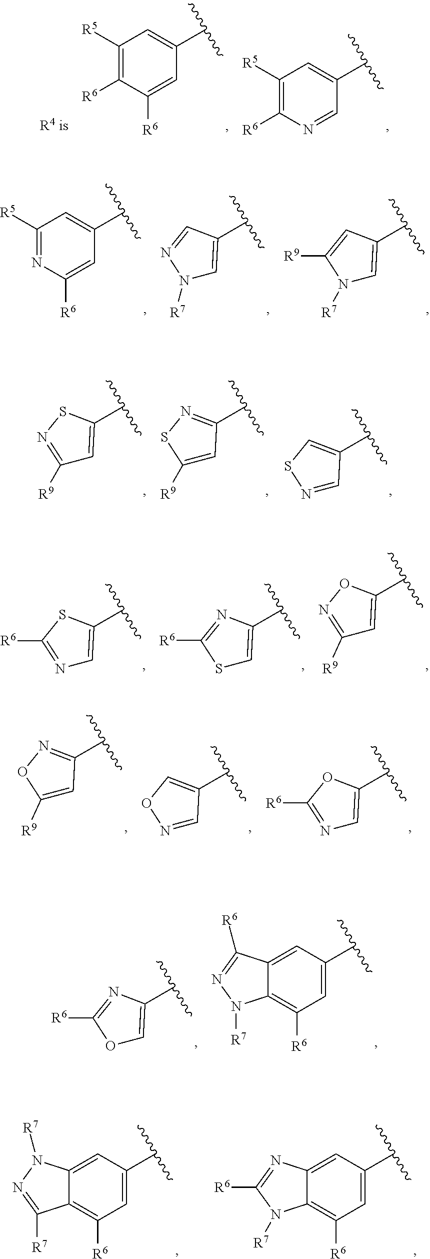

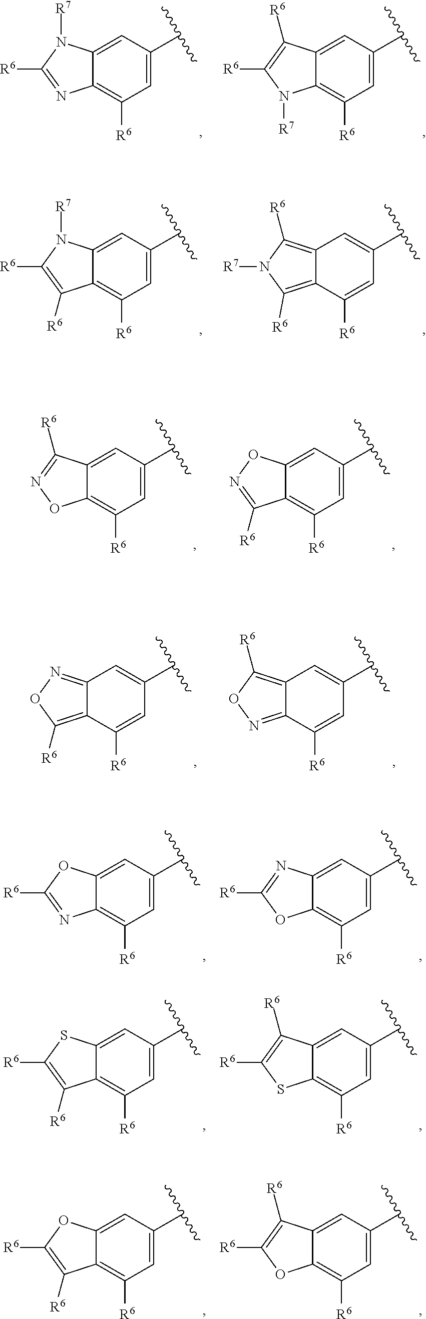

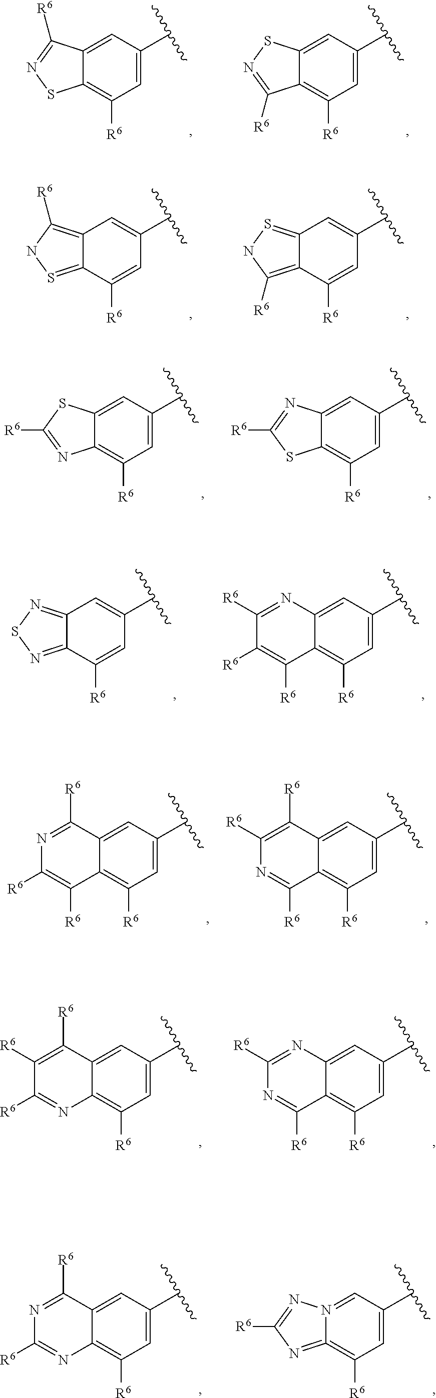

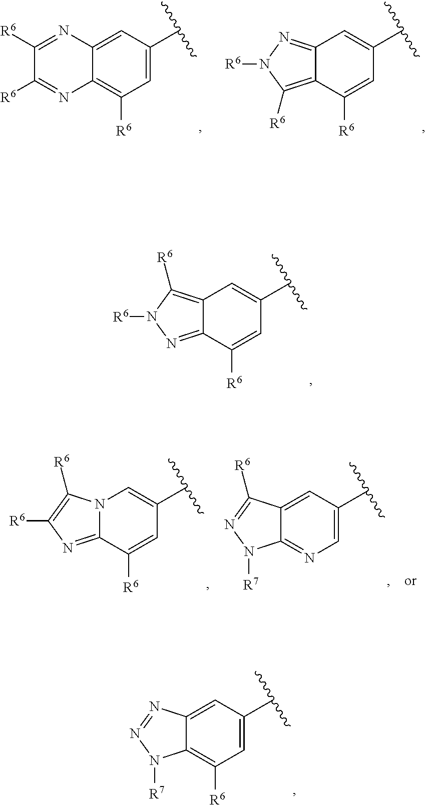

wherein “ [symbol] ” represents a point of attachment of R⁴ to the compound;
each R⁵ and each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl, wherein any C₁-C₄ alkyl or C₁-C₄ alkylene portion of R⁷ is optionally substituted with one or more substituents independently selected from halo and —CN;
R⁸ is hydrogen, —C₁-C₄ alkyl, or a 4-6 membered saturated heterocycle;
R⁹ is hydrogen, halo, —CN, —C₁-C₄ alkyl optionally substituted with halo, —O—C₁-C₄ alkyl optionally substituted with halo, or an optionally substituted phenyl;
m is 0, 1, 2, 3 or 4;
n is 0, 1, 2 or 3; and
m+n is 1, 2, 3, or 4,
   wherein any hydrogen atom is optionally replaced with deuterium.

In some embodiments, the present disclosure provides a compound having structural formula A:

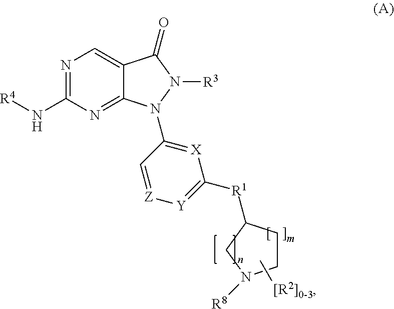

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged C₃-C₅ cycloalkyl ring, or one R² and R⁸ are optionally taken together to form a bridged 3-5 membered ring;
R³ is —C₁-C₃ alkyl or —CH₂—CH═CH₂;
R⁴ is

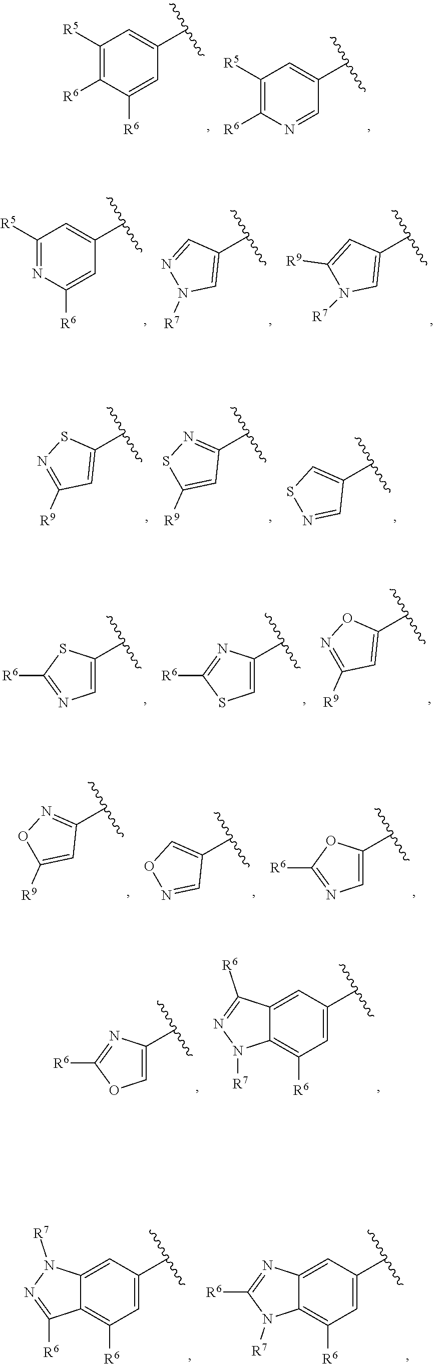

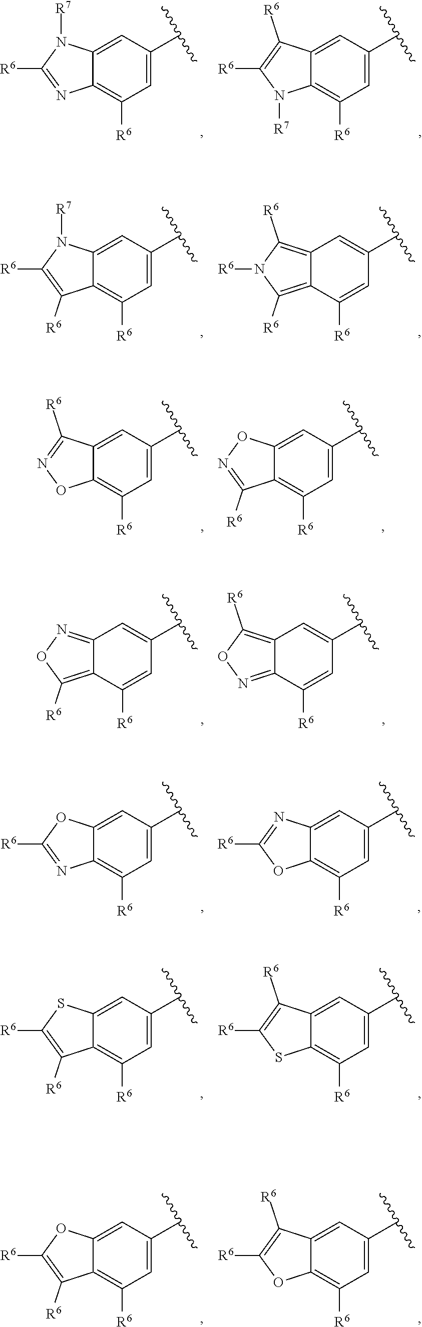

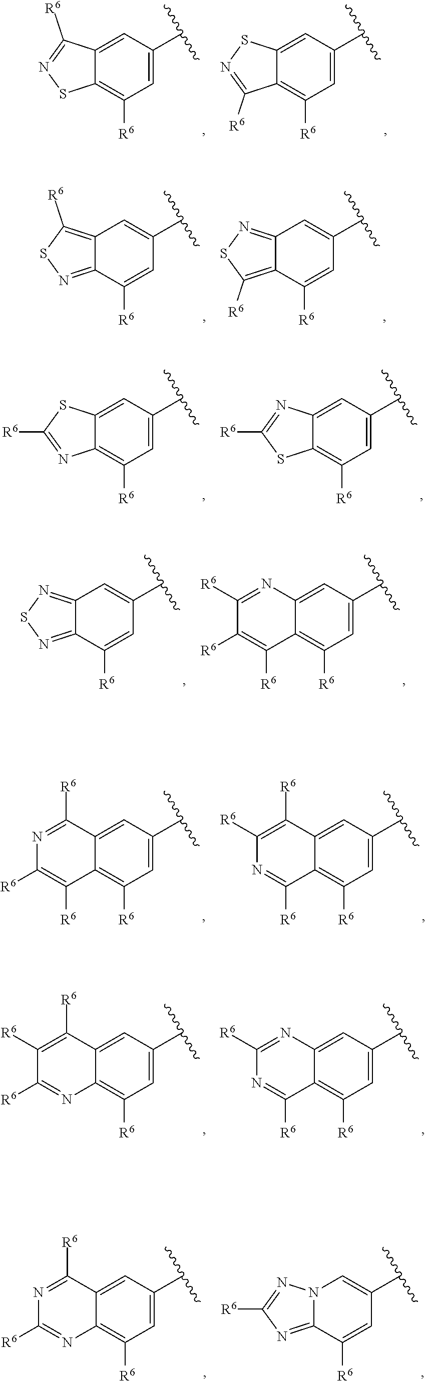

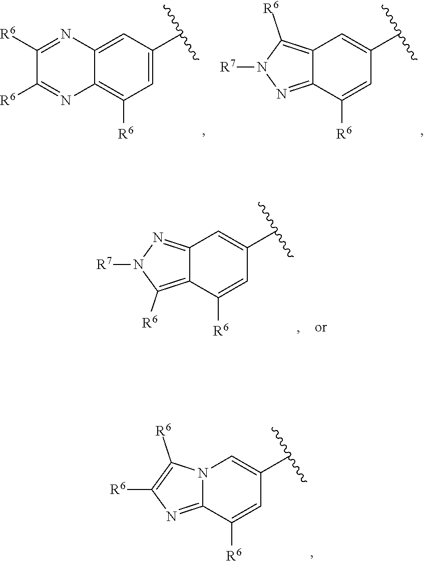

wherein “ [symbol] ” represents a point of attachment of R⁴ to the compound;
each R⁵ and each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl wherein any C₁-C₄ alkyl or C₁-C₄ alkylene portion of R⁷ is optionally substituted with one or more substituents independently selected from halo and —CN;
R⁸ is hydrogen, —C₁-C₄ alkyl, or a 4-6 membered saturated heterocycle;
R⁹ is hydrogen, halo, —CN, —C₁-C₄ alkyl optionally substituted with halo, —O—C₁-C₄ alkyl optionally substituted with halo, or an optionally substituted phenyl;
m is 0, 1, 2 or 3;
n is 0, 1, 2 or 3; and
m+n=1, 2, 3, or 4,
   wherein any hydrogen atom in Formula A is optionally substituted with deuterium.

In some embodiments, the present disclosure provides a compound having structural formula I:

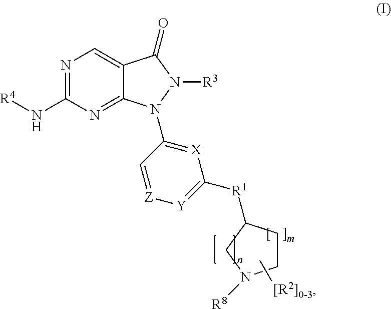

or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged C₃-C₅ cycloalkyl ring, or one R² and R⁸ are optionally taken together to form a bridged 3-5 membered ring;
R³ is —C₁-C₃ alkyl or —CH₂—CH—CH₂;
R⁴ is

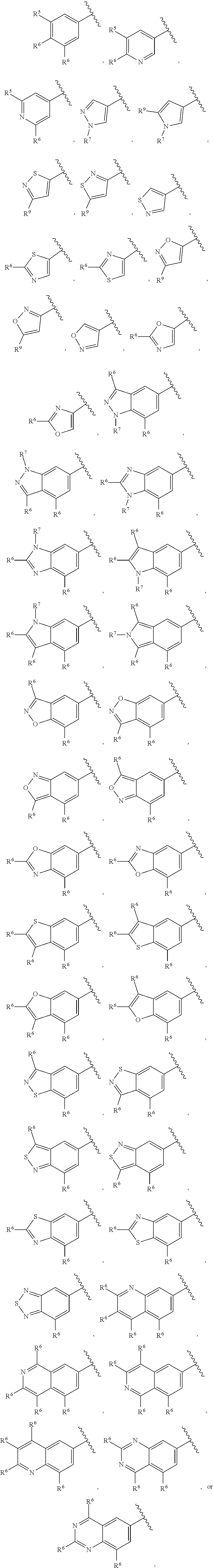

wherein “ [symbol] ” represents a point of attachment of R⁴ to the compound;
each R⁵ and each R⁶ is independently hydrogen, halo, —CN, —C₁-C₄ alkyl optionally substituted with halo, or —O—(C₁-C₄ alkyl) optionally substituted with halo, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, or —C₁-C₄ alkylene-O—C₁-C₄ alkyl, wherein any C₁-C₄ alkyl or C₁-C₄ alkylene portion of R⁷ is optionally substituted with one or more substituents independently selected from halo and —CN;
R⁸ is hydrogen or —C₁-C₄ alkyl;
R⁹ is hydrogen, halo, —CN, —C₁-C₄ alkyl optionally substituted with halo, —O—C₁-C₄ alkyl optionally substituted with halo, or an optionally substituted phenyl;
m is 0 or 1;
n is 0, 1, 2 or 3; and
m and n are not simultaneously 0.

As defined generally above and discussed throughout, each of X, Y and Z is independently CH or N.

In some embodiments, each of X, Y and Z is CH (i.e., the ring comprising X, Y, and Z is phen-2,6-diyl). In some embodiments, X is N and each of Y and Z is CH (i.e., the ring is pyridin-2,6-diyl). In some embodiments, X and Y is N and Z is CH (i.e., the ring is pyrimidin-2,6-diyl). In some embodiments, each of X and Z is N and Y is CH (i.e., the ring is pyrazin-2,6-diyl).

As defined generally above and discussed throughout, R¹ is —O—, —NH—, or —N(C₁-C₃ alkyl)-.

In some embodiments, R¹ is —O—. In some embodiments, R¹ is —NH—. In some embodiments, R¹ is —N(CH₃)—. In some embodiments, R¹ is —N(CH₂CH₃)—. In some embodiments, R¹ is —N(CH₂CH₂CH₃)—.

As defined generally above and discussed throughout, each R², if present, is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged or fused C₃-C₅ cycloalkyl ring, or one R² and R⁸ are optionally taken together to form a bridged or fused 3-5 membered ring.

The spiro-fused, fused, or bridged ring in the definition of R² refers to the ring formed by taking the two R² or the R² and R⁸ substituents together and how that ring is attached to the ring depicted as

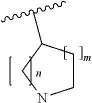

in Formula I. Examples of such spiro-fused, fused and bridged ring systems formed by taking two R² or one R² and one R⁸ together include, but are not limited to:

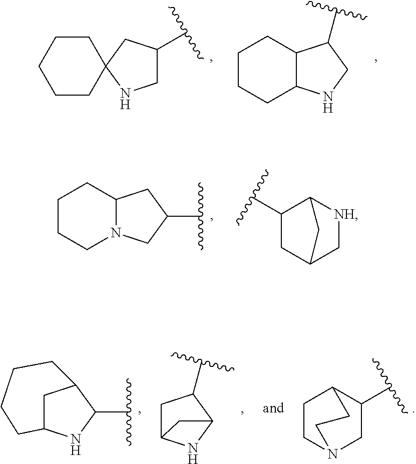

In some embodiments, R² is absent. In some embodiments, one R² and R⁸ are taken together with the ring to which they are bound to form

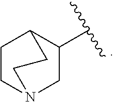 [SMILES: *C1CN2CCC1CC2]

In some embodiments, one R² and R⁸ are taken together with the ring to which they are bound to form

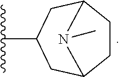 [SMILES: *C1CC2CCC(C1)N2C]

In some embodiments, one R² and R⁸ are taken together with the ring to which they are bound to form

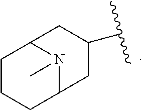 [SMILES: *C1CC2CCCC(C1)N2C]

As defined generally above and discussed throughout, R³ is —C₁-C₃ alkyl or —CH₂—CH═CH₂. In some embodiments, R³ is —CH₂—CH═CH₂. In some embodiments, R³ is methyl. In some embodiments, R³ is ethyl. In some embodiments, R³ is n-propyl.

As defined generally above and discussed throughout, R⁴ is

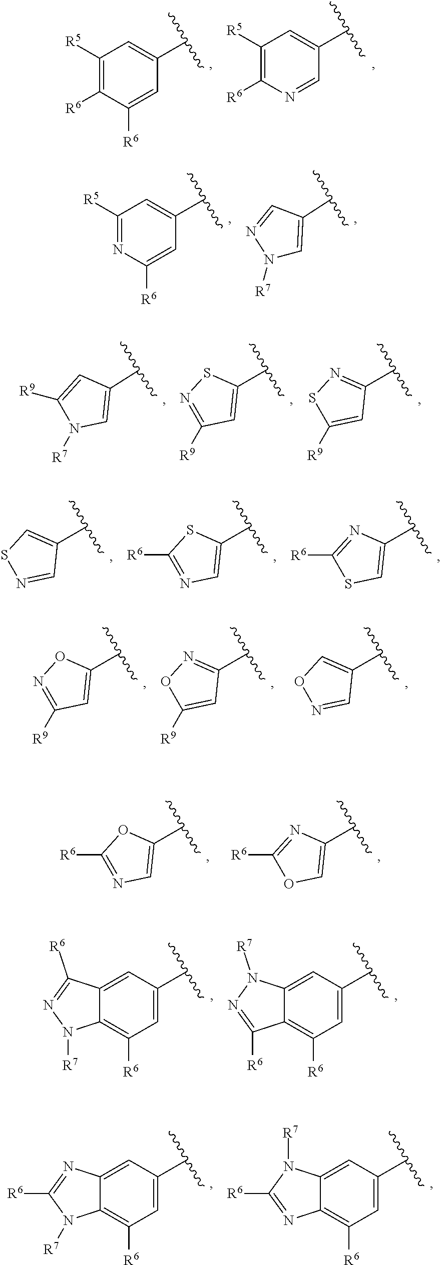

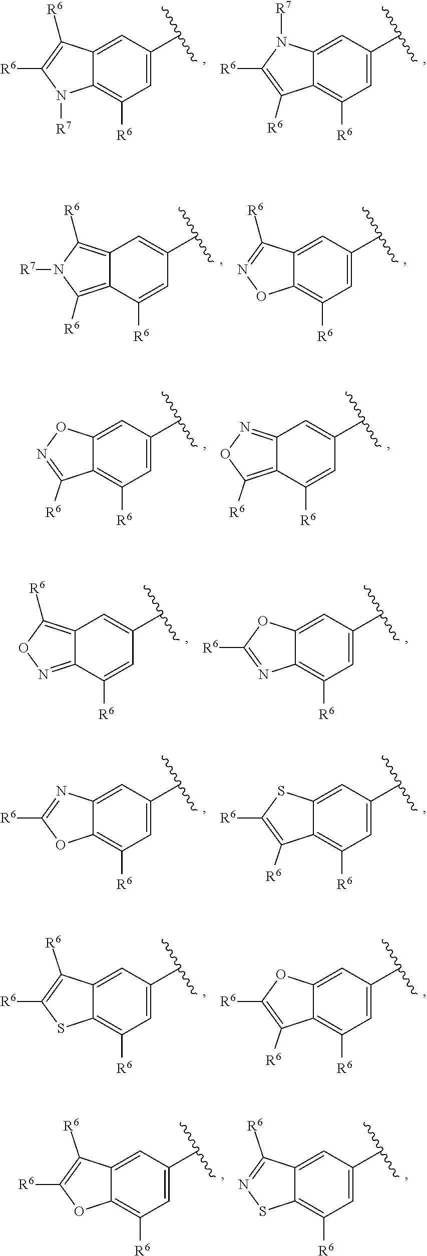

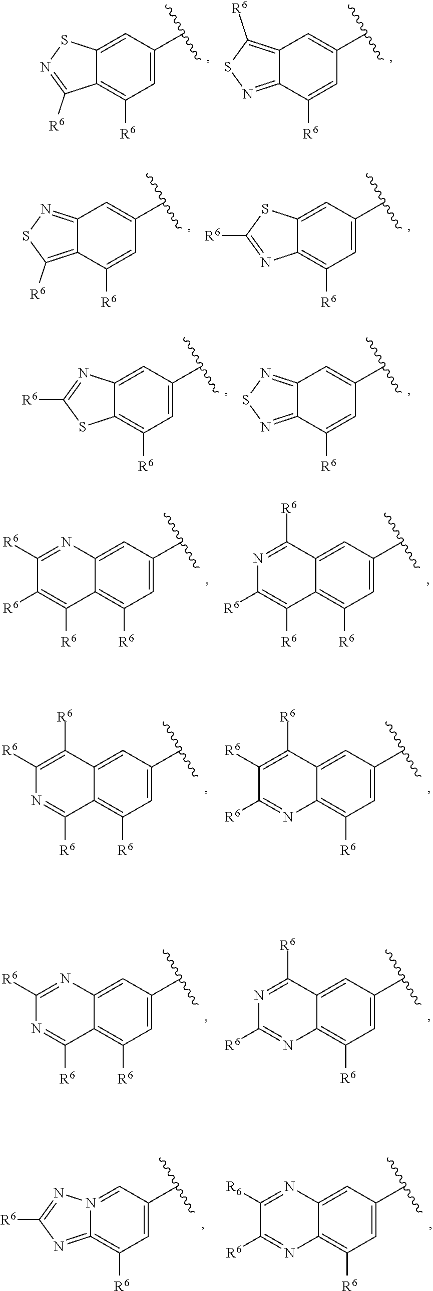

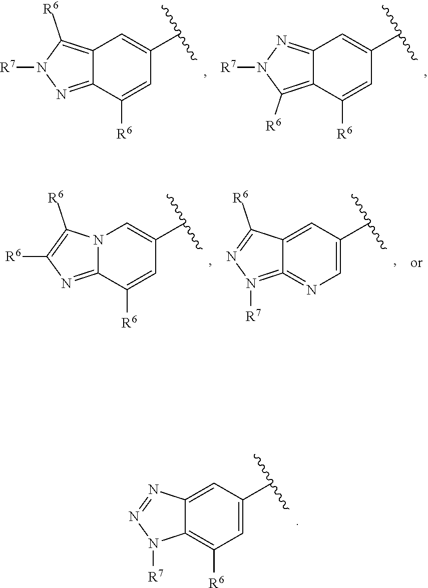

In some embodiments, R⁴ is

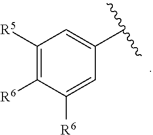

In some aspects of this embodiment, R⁵ is hydrogen. In some aspects of either of these embodiments, R⁵ is C₁-C₄ alkyl. In some aspects of this embodiment, R⁵ is methyl. In some aspects of this embodiment, R⁵ is —CN. In some aspects of either of these embodiments, R⁵ is halo. In some aspects of this embodiment, R⁵ is chloro. In some aspects of this embodiment, R⁵ is fluoro. In some aspects of this embodiment, R⁵ is bromo. In some aspects of either of these embodiments, R⁵ is —O—(C₁-C₄ alkyl) optionally substituted with halo. In some aspects of either of these embodiments, R⁵ is —O—(C₁-C₄ alkyl) optionally substituted with fluoro. In some aspects of this embodiment, R⁵ is —OCF₃. In some aspects of this embodiment, each R⁶ is hydrogen. In some aspects of this embodiment, one R⁶ is hydrogen and the other R⁶ is halo. In some aspects of this embodiment, one R⁶ is hydrogen and the other R⁶ is chloro. In some aspects of this embodiment, one R⁶ is hydrogen and the other R⁶ is fluoro. In some aspects of this embodiment, one R⁶ is hydrogen and the other R⁶ is bromo. In some aspects of this embodiment, one R⁶ is hydrogen and the other R⁶ is —C₁-C₄ alkyl optionally substituted with halo. In some aspects of this embodiment, one R⁶ is hydrogen and the other R⁶ is —C₁-C₄ alkyl optionally substituted with fluoro. In some aspects of this embodiment, one R^{e} is hydrogen and the other R⁶ is —CH₃. In some aspects of this embodiment, one R⁶ is hydrogen and the other R⁶ is —CF₃. In some aspects of this embodiment, one R⁶ is hydrogen and the other R⁶ is —O(C₁-C₄ alkyl) optionally substituted with halo. In some aspects of this embodiment, one R⁶ is hydrogen and the other R⁶ is —O(C₁-C₄ alkyl) optionally substituted with fluoro. In some aspects of this embodiment, one R⁶ is hydrogen and the other R⁶ is —OCH₃. In some aspects of this embodiment, one R⁶ is hydrogen and the other R⁶ is —OCH₂CF₃. In some aspects of this embodiment, one R⁶ is hydrogen and the other R⁶ is —CN. In some aspects of this embodiment, R⁵ and the R⁶ bound to an adjacent ring atom are taken together to form methylenedioxy. In some aspects of this embodiment, one R⁶ is hydrogen and the other R⁶ is cyclopropylmethoxy. In some aspects of this embodiment, one R⁶ is hydrogen and the other R⁶ is morpholinyl. In some aspects of this embodiment, one R⁶ is methyl and the other R⁶ is morpholinyl. In some aspects of this embodiment, one R⁶ is hydrogen and the other R⁶ is thiomorpholinyl. In some aspects of this embodiment, one R⁶ is hydrogen and the other R⁶ is 2-methylpropan-1-yloxy. In some aspects of this embodiment, one R⁶ is hydrogen and the other R⁶ is 1-methyl-1H-pyrazolyl. In some aspects of this embodiment, one R⁶ is hydrogen and the other R⁶ is phenyl optionally substituted with halo. In some aspects of this embodiment, one R⁶ is hydrogen and the other R⁶ is ethyloxy optionally substituted with halo.

In some embodiments, R⁴ is any one of phenyl, 3-methylphenyl, 3-chloro-5-bromophenyl, 3,4-dichlorophenyl, 4-bromophenyl, 4-chlorophenyl, 4-fluorophenyl, 4-(1-methylpiperidin-4-yl)-5-methylphenyl, 4-trifluoromethoxyphenyl, 3-methyl-4-trifluoromethoxyphenyl, 4-(2,2,2-trifluoroethan-1-yl)oxyphenyl, 3-methyl-4-(2,2,2-trifluoroethan-1-yl)oxyphenyl, 4-trifluoromethylphenyl, 3-methyl-4-chlorophenyl, 3-methyl-4-fluorophenyl, 3-methyl-5-fluorophenyl, 3-methyl-4-cyanophenyl, 3-methyl-4-methoxyphenyl, 3-methyl-4-(2,2,2-trifluoroethan-1-yloxy)phenyl, or 3,4-methylenedioxyphenyl.

In some embodiments, R⁴ is any one of 4-(cyclopropylmethyloxy)phenyl, 4-(morpholin-4-yl)phenyl, 4-(thiomorpholin-4-yl)phenyl, 4-(2-methylpropan-1-yloxy)phenyl, 3-(1-methyl-1H-pyrazol-4-yl)phenyl, 4-(1-methyl-1H-pyrazol-4-yl)phenyl, 3-methyl-4-(morpholin-4-yl)phenyl, 4-(phenyl)phenyl, 3-bromophenyl, 3-cyanophenyl, 4-(2-fluoroethan-1-yloxy)phenyl, 4-cyanophenyl, 4-(1,1-dioxothiazinan-4-yl)phenyl, 3-(pyridin-3-yl)phenyl, 3-(pyrimidin-5-yl)phenyl, 3-(1H-imidazol-1-yl)phenyl, 3-(1H-pyrazol-1-yl)phenyl, 3-(d3-methyl)phenyl, 4-(3-fluoropropan-1-yloxy)phenyl or 4-ethoxyphenyl.

In some embodiments, R⁴ is

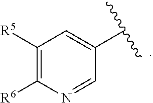

In some embodiments, R⁴ is

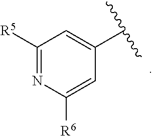

In some aspects of either of these two embodiments, R⁵ is hydrogen. In some aspects of either of these embodiments, R⁵ is halo. In some aspects of either of these embodiments, R⁵ is chloro. In some aspects of either of these embodiments, R⁵ is fluoro. In some aspects of either of these embodiments, R⁵ is cyano. In some aspects of either of these embodiments, R⁵ is C₁-C₄ alkyl optionally substituted with halo. In some aspects of either of these embodiments, R⁵ is C₁-C₄ alkyl optionally substituted with fluoro. In some aspects of either of these embodiments, R⁵ is —O—C₁-C₄ alkyl. In some aspects of either of these embodiments, R⁵ is methoxy. In some aspects of either of these embodiments, R⁵ is methyl. In some aspects of either of these embodiments, R⁵ is —CF₃. In some aspects of either of these embodiments, R⁶ is hydrogen. In some aspects of either of these embodiments, R⁶ is methyl. In some aspects of either of these embodiments, R⁶ is —O—C₁-C₄ alkyl. In some aspects of either of these embodiments, R⁶ is methoxy. In some aspects of either of these embodiments, R⁶ is —O—C₁-C₄ haloalkyl. In some aspects of either of these embodiments, R⁶ is 2-fluoroethoxy.

In some embodiments, R⁴ is any one of 2-methylpyridin-4-yl, 6-methylpyridin-3-yl, 2,6-dimethylpyridin-4-yl, 2-trifluoromethyl-6-methylpyridin-4-yl, or 2-trifluoromethylpyridin-4-yl.

In some embodiments, R⁴ is any one of pyridin-3-yl, 2-methylpyridin-5-yl, 2-methoxypyridin-4-yl, 2-methoxypyridin-5-yl, 3-chloropyridin-5-yl, 3-fluoropyridin-5-yl, 3-cyanopyridin-5-yl, 3-methylpyridin-5-yl, or 3-methoxypyridin-5-yl.

In some embodiments, R⁴ is 2-(2-fluoroethoxy)pyridin-5-yl.

In some embodiments, R⁴ is

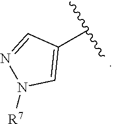

In some aspects of this embodiment, R⁷ is C₁-C₄ alkyl optionally substituted with halo. In some aspects of this embodiment, R⁷ is C₁-C₄ alkyl optionally substituted with fluoro. In some aspects of this embodiment, R⁷ is methyl. In some aspects of this embodiment, R⁷ is ethyl. In some aspects of this embodiment, R⁷ is propyl. In some aspects of this embodiment, R⁷ is isopropyl. In some aspects of this embodiment, R⁷ is —CH₂CH(CH₃)₂. In some aspects of this embodiment, R⁷ is —CH₂C(CH₃)₂F. In some aspects of this embodiment, R⁷ is —CH₂CH₂CF₃. In some aspects of this embodiment, R⁴ is any one of 1-methyl-1H-pyrazol-4-yl, 1-propyl-1H-pyrazol-4yl, 1-isopropyl-1H-pyrazol-4-yl, 1-(2,2-dimethylethan-1-yl)pyrazol-4-yl, 1-(2-fluoro-2,2-dimethylethan-1-yl)-1H-pyrazol-4-yl, or 1-(3,3,3-trifluoropropan-1-yl)-1H-pyrazol-4-yl.

In some embodiments, R⁴ is

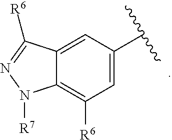

In some aspects of this embodiment, each R⁶ is hydrogen. In some aspects of this embodiment, R⁷ is hydrogen. In some aspects of this embodiment, R⁷ is C₁-C₄ alkyl optionally substituted with halo. In some aspects of this embodiment, R⁷ is C₁-C₄ alkyl optionally substituted with fluoro. In some aspects of this embodiment, R⁷ is methyl. In some aspects of this embodiment, R⁷ is ethyl. In some aspects of this embodiment, R⁷ is propyl. In some aspects of this embodiment, R⁷ is isopropyl. In some aspects of this embodiment, R⁷ is —CH₂CH(CH₃)₂. In some aspects of this embodiment, R⁷ is —CH₂C(CH₃)₂F. In some aspects of this embodiment, R⁷ is —CH₂CH₂CF₃. In some aspects of this embodiment, R⁷ is cyclopropyl. In some aspects of this embodiment, R⁷ is isobutyl. In some aspects of this embodiment, R⁷ is 3-fluoropropyl. In some aspects of this embodiment, R⁴ is 1-methyl-1H-indazol-5-yl. In some embodiments, R⁴ is selected from 1H-indazol-5-yl, 1-methyl-1H-indazol-5-yl, 1-ethyl-1H-indazol-5-yl, 1-isopropyl-1H-indazol-5-yl, 1-propyl-1H-indazol-5-yl, 1-(3-fluoropropan-1-yl)-1H-indazol-5-yl, 1-isobutyl-1H-indazol-5-yl, and 1-cyclopropyl-1H-indazol-5-yl.

In some embodiments, when R⁴ is

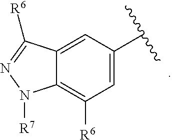

one R⁶ is hydrogen and the other R⁶ is methyl or halo. In some aspects of this embodiment, R⁴ is 3-chloro-1H-indazol-5-yl, 3-methyl-1H-indazol-5-yl, or 1,3-dimethyl-1H-indazol-5-yl.

In some embodiments, R⁴ is

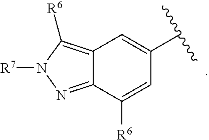

In some aspects of this embodiment, R⁷ is hydrogen. In some aspects of this embodiment, R⁷ is methyl. In some aspects of this embodiment, R⁷ is ethyl. In some aspects of this embodiment, R⁷ is t-butyl. In some aspects of this embodiment, each R⁶ is hydrogen. In some aspects of this embodiment, R⁴ is 2-methyl-2H-indazol-5-yl, or 2-ethyl-2H-indazol-5-yl. In some aspects of this embodiment, R⁴ is 2-(t-butyl)-2H-indazol-5-yl.

In some embodiments, R⁴ is

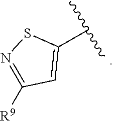

In some aspects of this embodiment, R⁹ is an optionally substituted phenyl. In some aspects of this embodiment, R⁹ is unsubstituted phenyl. In some aspects of this embodiment, R⁴ is 3-phenylisothiazol-5-yl.

In some embodiments, R⁴ is

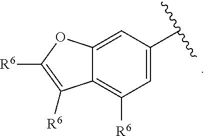

In some embodiments, R⁴ is

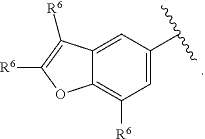

In some aspects of each of these embodiments, each R⁶ is hydrogen. In some aspects of each of these embodiments, two R⁶ are hydrogen and the other R⁶ is other than hydrogen. In some aspects of each of these embodiments, one R⁶ is hydrogen and the other two R⁶ are other than hydrogen. In some aspects of this embodiment, R⁴ is benzofuran-6-yl. In some aspects of this embodiment, R⁴ is benzofuran-5-yl.

In some embodiments, R⁴ is

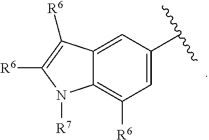

In some aspects of this embodiment, R⁷ is C₁-C₄ alkyl. In some aspects of this embodiment, R⁷ is methyl. In some aspects of this embodiment, R⁷ is —C(O)CH₃. In some aspects of this embodiment, each R⁶ is hydrogen. In some aspects of this embodiment, two R⁶ are hydrogen and the other R⁶ is other than hydrogen. In some aspects of this embodiment, one R⁶ is hydrogen and the other two R⁶ are other than hydrogen. In some aspects of this embodiment, R⁴ is 1-methyl-1H-indol-5-yl. In some aspects of this embodiment, R⁴ is 1-acetyl-1H-indol-5-yl.

In some embodiments, R⁴ is

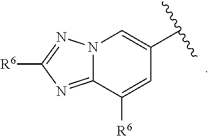

In some aspects of this embodiment, each R⁶ is hydrogen. In some aspects of this embodiment, R⁴ is 1,2,4-triazolo[1,5-a]pyridin-6-yl.

In some embodiments, R⁴ is

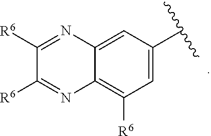

In some aspects of this embodiment, each R⁶ is hydrogen. In some aspects of this embodiment, R⁴ is quinoxalin-6-yl.

In some embodiments, R⁴ is selected from any one of

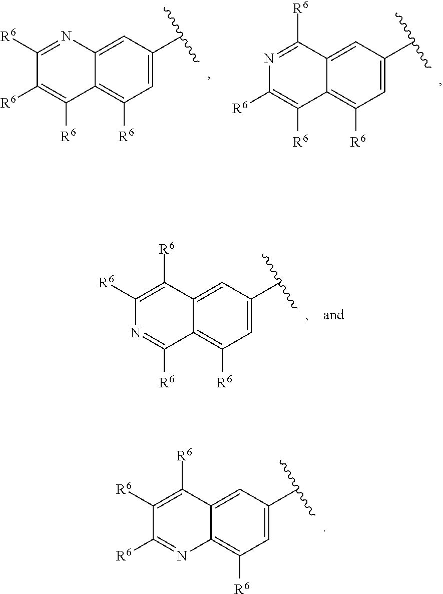

In some aspects of this embodiment, each R⁶ is hydrogen. In some aspects of this embodiment, R⁴ is quinolin-6-yl, quinolin-7-yl, isoquinolin-6-yl, or isoquinolin-7-yl.

In some embodiments, R⁴ is

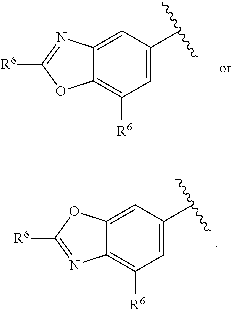

In some aspects of these embodiments, each R⁶ is hydrogen. In some aspects of these embodiments, one R⁶ is hydrogen and the other R⁶ is methyl. In some aspects of these embodiments, R⁴ is 2-methylbenzo[d]oxazol-5-yl or 2-methylbenzo[d]oxazol-6-yl.

In some embodiments, R⁴ is

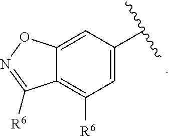

In some aspects of these embodiments, each R⁶ is hydrogen. In some aspects of these embodiments, R⁴ is benzo[d]isoxazol-6-yl.

In some embodiments, R⁴ is

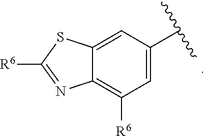

In some aspects of this embodiment, each R⁶ is hydrogen. In some aspects of this embodiment, one R⁶ is hydrogen and the other R⁶ is methyl. In some aspects of this embodiment, R⁴ is benzo[d]thiazol-6-yl or 2-methylbenzo[d]thiazol-6-yl.

In some embodiments, R⁴ is

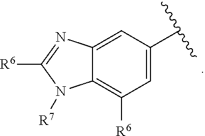

In some aspects of this embodiment, each R⁶ is hydrogen. In some aspects of this embodiment, R⁷ is methyl. In some aspects of this embodiment, R⁴ is 1-methyl-1H-benzo[d]imidazol-5-yl. In some aspects of this embodiment, R⁷ is isopropyl. In some aspects of this embodiment, R⁴ is 1-isopropyl-1H-benzo[d]imidazol-5-yl.

In some embodiments, R⁴ is

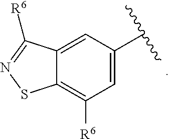

In some aspects of this embodiment, each R⁶ is hydrogen. In some aspects of this embodiment, R⁴ is benzo[d]thiazol-5-yl.

In some embodiments, R⁴ is

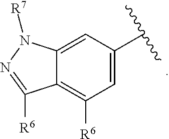

In some aspects of this embodiment, each R⁶ is hydrogen. In some aspects of this embodiment, R⁷ is methyl. In some aspects of this embodiment, R⁴ is 1-methyl-1H-indazol-6-yl.

In some embodiments, R⁴ is

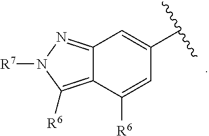

In some aspects of this embodiment, each R⁶ is hydrogen. In some aspects of this embodiment, R⁷ is methyl. In some aspects of this embodiment, R⁴ is 2-methyl-2H-indazol-6-yl.

In some embodiments, R⁴ is

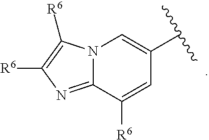

In some aspects of this embodiment, each R⁶ is hydrogen. In some aspects of this embodiment, R⁴ is imidazo[1,2-a]pyridin-6-yl.

In some embodiments, R⁴ is

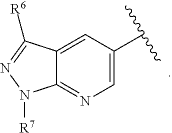

In some aspects of this embodiment, R⁶ is hydrogen. In some aspects of this embodiment, R⁷ is C₁-C₄ alkyl. In some aspects of this embodiment, R⁷ is isopropyl. In some aspects of this embodiment, R⁴ is 1-isopropyl-1H-pyrazolo[3,4-b]pyridin-5-yl.

In some embodiments, R⁴ is

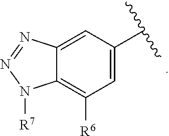

In some aspects of this embodiment, R⁶ is hydrogen. In some aspects of this embodiment, R⁷ is C₁-C₄ alkyl. In some aspects of this embodiment, R⁷ is isopropyl. In some aspects of this embodiment, R⁴ is 1-isopropyl-1H-benzo[d][1,2,3]triazol-5-yl.

As defined generally above and discussed throughout, R⁵ is hydrogen, halo, —CN, —C₁-C₄ alkyl optionally substituted with halo, or —O—(C₁-C₄ alkyl) optionally substituted with halo, or R⁵ and an R⁶ on an adjacent ring atom are taken together to form a 4-7 membered saturated heterocyclic or cycloalkyl ring that is fused to R⁴. In some embodiments, R⁵ is hydrogen. In some embodiments, R⁵ is halo. In some embodiments, R⁵ is chloro. In some embodiments, R⁵ is fluoro. In some embodiments, R⁵ is C₁-C₄ alkyl optionally substituted with halo. In some embodiments, R⁵ is C₁-C₄ alkyl optionally substituted with fluoro. In some embodiments, R⁵ is methyl. In some embodiments, R⁵ is —CF₃. In some embodiments, R⁵ and an R⁶ on an adjacent ring atom are taken together to form a 4-7 membered saturated heterocyclic or cycloalkyl ring that is fused to R⁴. In some embodiments, R⁵ and an R⁶ on an adjacent ring atom are taken together to form a methylenedioxy that is fused to R⁴.

As defined generally above and discussed throughout, each R⁶ is independently hydrogen, halo, —CN, —C₁-C₄ alkyl optionally substituted with halo, or —O—(C₁-C₄ alkyl) optionally substituted with halo, wherein no more than two R⁶ are other than hydrogen. In some embodiments, no more than one R⁶ is other than hydrogen. In some embodiments, each R⁶ is hydrogen. In some embodiments, one R⁶ is halo. In some embodiments, one Reis chloro. In some embodiments, one R⁶ is fluoro. In some embodiments, one R⁶ is bromo. In some embodiments, one R⁶ is —C₁-C₄ alkyl optionally substituted with halo. In some embodiments, one R⁶ is —C₁-C₄ alkyl optionally substituted with fluoro. In some embodiments, one R⁶ is —CH₃. In some embodiments, one R⁶ is —CF₃. In some aspects of this embodiment, one R⁶ is hydrogen and the other R⁶ is halo. In some embodiments, one R⁶ is —O(C₁-C₄ alkyl) optionally substituted with halo. In some embodiments, one R⁶ is —O(C₁-C₄ alkyl) optionally substituted with fluoro. In some embodiments, one R⁶ is —OCH₃. In some embodiments, one R⁶ is —OCH₂CF₃. In some embodiments, one R⁶ is —CN.

As defined generally above and discussed throughout, R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl, wherein any C₁-C₄ alkyl or C₁-C₄ alkylene portion of R⁷ is optionally substituted with one or more substituents independently selected from halo and —CN. In some embodiments, R⁷ is C₁-C₄ alkyl optionally substituted with halo. In some embodiments, R⁷ is C₁-C₄ alkyl optionally substituted with fluoro. In some embodiments, R⁷ is methyl. In some embodiments, R⁷ is ethyl. In some embodiments, R⁷ is propyl. In some embodiments, R⁷ is isopropyl. In some embodiments, R⁷ is —CH₂CH(CH₃)₂. In some embodiments, R⁷ is —CH₂C(CH₃)₂F. In some embodiments, R⁷ is —CH₂CH₂CF₃. In some embodiments, R⁷ is —CH₂CH₂CH₂F₃. In some embodiments, R⁷ is cyclopropyl. In some embodiments, R⁷ is —C(O)CH₃.

As defined generally above and discussed throughout, R⁸ is hydrogen, —C₁-C₄ alkyl, or a 4-6 membered saturated heterocycle. In some embodiments, R⁸ is hydrogen. In some embodiments, R⁸ is methyl. In some embodiments, R⁸ is ethyl. In some embodiments, R⁸ is propyl. In some embodiments, R⁸ is oxetan-3-yl.

As defined generally above and discussed throughout, R⁹ is hydrogen, halo, —CN, —C₁-C₄ alkyl optionally substituted with halo, —O—C₁-C₄ alkyl optionally substituted with halo, or an optionally substituted phenyl. In some embodiments, R⁹ is unsubstituted phenyl.

As defined generally above and discussed throughout, the monocyclic heterocyclic ring represented by the structure:

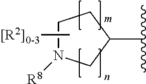

may comprise between 4 (when the sum of m+n is 1) and 7 (when the sum of m+n is 4) ring atoms. In some embodiments, that ring may be a spiro-fused bicyclic ring when two R² bound to the same carbon atom are taken together to form a C₃-C₇ cycloalkyl ring. In some embodiments, that ring may be a bridged bicyclic ring when two R² bound to different carbon atoms are taken together to form a C₃-C₅ cycloalkyl ring. In some embodiments, that ring may be a bridged bicyclic ring when one R² and R⁸ are taken together to form a 3-5 membered ring. In some embodiments, that ring may be a fused bicyclic ring when two R² bound to different carbon atoms are taken together to form a C₃-C₅ cycloalkyl ring. In some embodiments, that ring may be a fused bicyclic ring when one R² and R⁸ are taken together to form a 3-5 membered ring.

In some embodiments, the ring represented by the structure:

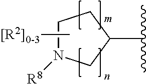

is a piperidinyl ring. In some aspects of these embodiments, the piperidinyl ring is a piperidin-3-yl ring (i.e., nis 1 and m is 2; or n is 3 and m is 0). In some aspects of these embodiments, the piperidinyl ring is a piperidin-4-yl ring (i.e., n is 2 and m is 1). In some aspects of these embodiments, the R⁸ substituent on such piperidinyl ring is hydrogen. In some aspects of these embodiments, the R⁸ substituent on such piperidinyl ring is methyl. In some aspects of these embodiments, the R⁸ substituent on such piperidinyl ring is trideuteromethyl (methyl-d3). In some aspects of these embodiments, the R⁸ substituent on such piperidinyl ring is ethyl. In some aspects of these embodiments, the R⁸ substituent on such piperidinyl ring is isopropyl. In some aspects of these embodiments, the R⁸ substituent on such piperidinyl ring is propyl. In some aspects of these embodiments, R² is absent. In some aspects of these embodiments, one or more R² are methyl. In some aspects of these embodiments, the piperidinyl ring is piperidinyl-4-yl, piperidinyl-3-yl, 1-methylpiperidin-4-yl, 1-d3-methylpiperidin-4-yl, 1-methylpiperidin-3-yl, 1-ethylpiperidin-4-yl, 1-propylpiperidin-4-yl, 1,2-dimethylpiperidin-4-yl, 2,2-dimethylpiperidin-4-yl, 2-methylpiperidin-4-yl, 1,2,2-trimethylpiperidin-4-yl, or 1-(oxetan-3-yl)piperindin-4-yl.

In some embodiments, the ring represented by the structure:

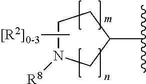

is a pyrrolidinyl ring. In some aspects of these embodiments, the pyrrolidinyl ring is a pyrrolidin-3-yl ring (i.e., n is 1 and m is 1; or n is 2 and m is 0). In some aspects of these embodiments, the R⁸ substituent on such pyrrolidinyl ring is hydrogen. In some aspects of these embodiments, the R⁸ substituent on such pyrrolidinyl ring is methyl. In some aspects of these embodiments, the R⁸ substituent on such pyrrolidinyl ring is ethyl. In some aspects of these embodiments, the R⁸ substituent on such pyrrolidinyl ring is isopropyl. In some aspects of these embodiments, R² is absent.

In some embodiments, the ring represented by the structure:

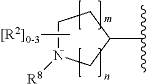

is a quinuclidinyl ring. In some aspects of these embodiments, the quinuclidinyl ring is a quinuclidin-3-yl ring (i.e., n is 1 and m is 2; or n is 3 and m is 0; and one R² and R⁸ are taken together to form a bridged 4 membered ring). In some aspects of these embodiments, the R⁸ substituent on such quinuclidinyl ring is hydrogen. In some aspects of these embodiments, the R⁸ substituent on such quinuclidinyl ring is methyl. In some aspects of these embodiments, the R⁸ substituent on such quinuclidinyl ring is ethyl. In some aspects of these embodiments, the R⁸ substituent on such quinuclidinyl ring is isopropyl. In some aspects of these embodiments, R² is absent.

In some embodiments, the ring represented by the structure:

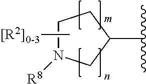

is an azabicyclo[3.2.1]octanyl ring. In some aspects of these embodiments, the azabicyclo[3.2.1]octanyl ring is an 8-azabicyclo[3.2.1]octan-3-yl ring (i.e., n is 1 and m is 2; or n is 3 and m is 0; and two R² bound to different carbon atoms are taken together to form a bridged C₄ cycloalkyl ring). In some aspects of these embodiments, the R⁸ substituent on such azabicyclo[3.2.1]octanyl ring is hydrogen. In some aspects of these embodiments, the R⁸ substituent on such azabicyclo[3.2.1]octanyl ring is methyl. In some aspects of these embodiments, the R⁸ substituent on such azabicyclo[3.2.1]octanyl ring is ethyl. In some aspects of these embodiments, the R⁸ substituent on such azabicyclo[3.2.1]octanyl ring is isopropyl. In some aspects of these embodiments, R² is absent.

In some embodiments, the ring represented by the structure:

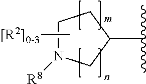

is an azabicyclo[3.3.1]nonanyl ring. In some aspects of these embodiments, the azabicyclo[3.3.1]nonanyl ring is a 9-azabicyclo[3.3.1]nonan-3-yl ring (i.e., n is 1 and m is 2; or n is 3 and m is 0; and two R² bound to different carbon atoms are taken together to form a bridged C₅ cycloalkyl ring). In some aspects of these embodiments, the R⁸ substituent on such azabicyclo[3.3.1]nonanyl ring is hydrogen. In some aspects of these embodiments, the R⁸ substituent on such azabicyclo[3.3.1]nonanyl ring is methyl. In some aspects of these embodiments, the R⁸ substituent on such azabicyclo[3.3.1]nonanyl ring is ethyl. In some aspects of these embodiments, the R⁸ substituent on such azabicyclo[3.3.1]nonanyl ring is isopropyl. In some aspects of these embodiments, R² is absent.

In some embodiments, the ring represented by the structure:

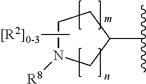

is an azabicyclo[2.2.2]octanyl ring. In some aspects of these embodiments, the azabicyclo[2.2.2]octanyl ring is a 2-azabicyclo[2.2.2]octan-5-yl ring (i.e., n is 1 and m is 2; or n is 3 and m is 0; and two R² bound to different carbon atoms are taken together to form a bridged C₄ cycloalkyl ring). In some aspects of these embodiments, the R⁸ substituent on such azabicyclo[2.2.2]octanyl ring is methyl.

In some embodiments, the ring represented by the structure:

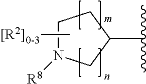

is an azepanyl ring. In some aspects of these embodiments, the azepanyl ring is azepan-4-yl (i.e., n is 2 and m is 2). In some aspects of these embodiments, the R⁸ substituent on such azepanyl ring is hydrogen. In some aspects of these embodiments, the R⁸ substituent on such azepanyl ring is methyl.

In some embodiments, the compound of formula I may be a compound, or a pharmaceutically acceptable salt thereof, selected from Table 1, which lists chemical structure for each compound and LCMS and ¹H NMR for those compounds that have therein been so analyzed. Chemical shifts are reported in ppm (δ) using the residual solvent as internal standard. Peak multiplicities given in Hz are expressed as follow: s, singlet; d, doublet; dd, doublet of doublets; ddd, doublet of doublet of doublets; t, triplet; dt, doublet of triplets; q, quartet; dq, doublet of quartets; p, pentet; h, heptet; m, multiplet; br s, broad singlet.

TABLE 1
Exemplary Compounds

| Cpd. | Structure | LCMS | ¹HNMR |
|---|---|---|---|
| 100 | 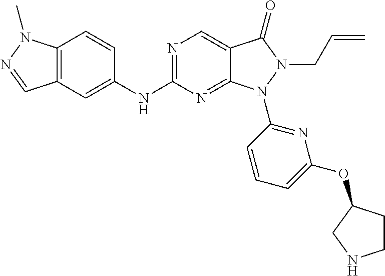 [SMILES: C=CCn1c(=O)c2cnc(Nc3ccc4c(cnn4C)c3)nc2n1-c1cccc(O[C@H]2CCNC2)n1] | 484.0 | 1H NMR (400 MHz, Methanol-d4) δ 8.82 (s, 1H), 8.53 (s, 1H), 8.20 (s, 1H), 7.97 (t, J = 7.9 Hz, 1H), 7.92 (s, 1H), 7.61-7.40 (m, 3H), 6.86 (d, J = 8.2 Hz, 1H), 5.84-5.70 (m, 1H), 5.65 (s, 1H), 5.11 (d, J = 10.3 Hz, 1H), 5.05-4.98 (m, 1H), 4.79-4.61 (m, 2H), 4.06 (s, 3H), 3.51 (s, 2H), 3.47-3.35 (m, 2H), 2.44-2.20 (m, 2H). One exchangeable proton not listed |
| 101 | 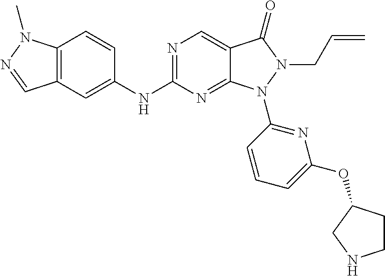 [SMILES: C=CCn1c(=O)c2cnc(Nc3ccc4c(cnn4C)c3)nc2n1-c1cccc(O[C@@H]2CCNC2)n1] | 484.0 | 1H NMR (400 MHz, Methanol-d4) δ 8.84 (s, 1H), 8.22 (s, 1H), 8.05-7.91 (m, 2H), 7.58 (dd, J = 9.0, 1.9 Hz, 1H), 7.55-7.50 (m, 2H), 6.85 (d, J = 8.2 Hz, 1H), 5.88-5.70 (m, 1H), 5.62 (s, 1H), 5.11 (d, J = 10.2 Hz, 1H), 5.01 (d, J = 18.1 Hz, 1H), 4.77-4.67 (m, 2H), 4.07 (s, 3H), 3.52-3.33 (m, 4H), 2.44-2.11 (m, 2H). Two exchangeable protons not listed. |
| 102 | 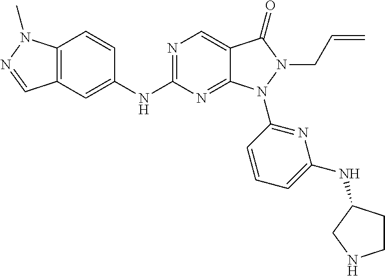 [SMILES: C=CCn1c(=O)c2cnc(Nc3ccc4c(cnn4C)c3)nc2n1-c1cccc(N[C@@H]2CCNC2)n1] | 483.0 | 1H NMR (400 MHz, DMSO-d6) δ 10.36 (s, 1H), 8.86 (s, 1H), 8.72 (s, 2H), 8.33 (s, 1H), 7.99 (s, 1H), 7.75 (s, 1H), 7.64-7.56 (m, 2H), 7.21 (d, J = 5.3 Hz, 1H), 7.05 (d, J = 7.5 Hz, 1H), 6.52 (d, J = 8.2 Hz, 1H), 5.78-5.63 (m, 1H), 5.07 (d, J = 10.2 Hz, 1H), 4.93 (d, J = 18.3 Hz, 1H), 4.61 (s, 2H), 4.40-4.32 (m, 1H), 4.03 (s, 3H), 3.35-3.19 (m, 3H), 3.03 (td, J = 11.0, 4.5 Hz, 1H), 2.21 (dq, J = 14.9, 8.0 Hz, 1H), 1.90 (td, J = 13.4, 7.4 Hz, 1H) |
| 103 | 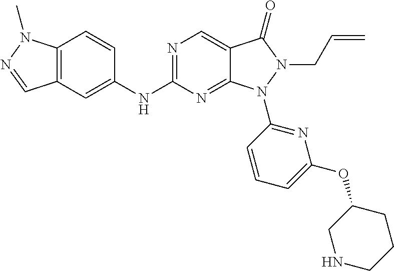 [SMILES: C=CCn1c(=O)c2cnc(Nc3ccc4c(cnn4C)c3)nc2n1-c1cccc(O[C@@H]2CCCNC2)n1] | 498.0 | 1H NMR (500 MHz, DMSO) δ 10.41 (s, 1H), 8.87 (s, 1H), 8.73 (s, 1H), 8.53 (s, 1H), 8.25 (s, 1H), 8.06 (s, 1H), 8.01 (s, 1H), 7.58 (s, 1H), 7.55 (d, J = 7.6 Hz, 1H), 6.83 (d, J = 8.0 Hz, 1H), 5.70 (ddt, J = 16.2, 10.3, 5.9 Hz, 1H), 5.25 (s, 1H), 5.05 (dd, J = 10.3, 1.3 Hz, 1H), 4.94 (dd, J = 17.1, 1.4 Hz, 1H), 4.58 (d, J = 5.6 Hz, 2H), 4.02 (s, 3H), 3.34 (s, 1H), 3.26 (s, 1H), 3.06 (d, J = 13.3 Hz, 2H), 1.90 (t, J = 13.4 Hz, 2H), 1.86-1.78 (m, 1H), 1.69 (s, 1H) |
| 104 | 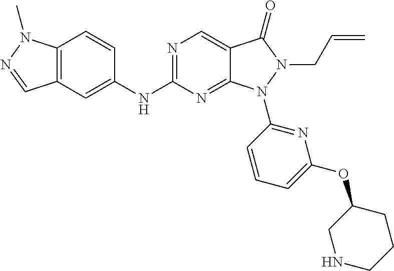 [SMILES: C=CCn1c(=O)c2cnc(Nc3ccc4c(cnn4C)c3)nc2n1-c1cccc(O[C@H]2CCCNC2)n1] | 498.0 | 1H NMR (500 MHz, DMSO) δ 10.41 (s, 1H), 8.88 (s, 1H), 8.73 (s, 1H), 8.52 (s, 1H), 8.27 (s, 1H), 8.07 (s, 1H), 8.02 (s, 1H), 7.59 (s, 1H), 7.56 (d, J = 7.6 Hz, 1H), 6.84 (d, J = 7.9 Hz, 1H), 5.71 (ddt, J = 16.2, 10.3, 5.9 Hz, 1H), 5.26 (s, 1H), 5.06 (dd, J = 10.3, 1.3 Hz, 1H), 4.95 (dd, J = 17.1, 1.4 Hz, 1H), 4.60 (s, 2H), 4.03 (s, 3H), 3.35 (s, 1H), 3.27 (s, 1H), 3.08 (s, 2H), 1.90 (d, J = 15.2 Hz, 2H), 1.87- 1.81 (m, 1H), 1.70 (s, 1H). |
| 105 | 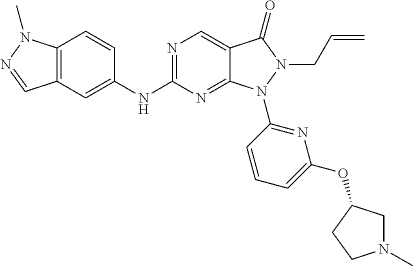 [SMILES: C=CCn1c(=O)c2cnc(Nc3ccc4c(cnn4C)c3)nc2n1-c1cccc(O[C@H]2CCN(C)C2)n1] | 498.3 | 1H NMR (500 MHz, Methanol-d4) δ 8.83 (s, 1H), 8.20 (s, 1H), 7.93 (s, 1H), 7.90 (t, J = 7.9 Hz, 1H), 7.58 (dd, J = 9.0, 1.9 Hz, 1H), 7.51 (d, J = 9.0 Hz, 1H), 7.44 (d, J = 7.6 Hz, 1H), 6.80 (d, J = 8.2 Hz, 1H), 5.81-5.69 (m, 1H), 5.44-5.38 (m, 1H), 5.12-5.07 (m, 1H), 5.01-4.96 (m, 1H), 4.75-4.71 (m, 3H), 4.07 (s, 3H), 2.91-2.79 (m, 3H), 2.66 (s, 1H), 2.49-2.42 (m, 1H), 2.36 (s, 3H), 1.98 (dtd, J = 14.3, 7.6, 2.9 Hz, 1H). |
| 106 | 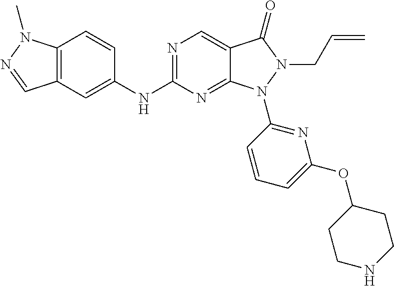 [SMILES: C=CCn1c(=O)c2cnc(Nc3ccc4c(cnn4C)c3)nc2n1-c1cccc(OC2CCNCC2)n1] | 498.0 | 1H NMR (500 MHz, DMSO-d6) δ 10.40 (s, 1H), 8.88 (s, 1H), 8.64-8.46 (m, 2H), 8.25 (s, 1H), 8.08-8.02 (m, 1H), 8.01 (s, 1H), 7.66-7.57 (m, 2H), 7.53 (d, J = 7.6 Hz, 1H), 6.85 (d, J = 8.1 Hz, 1H), 5.71 (ddt, J = 16.2, 10.3, 5.8 Hz, 1H), 5.19 (tt, J = 7.3, 3.5 Hz, 1H), 5.06 (dd, J = 10.3, 1.3 Hz, 1H), 4.94 (dd, J = 17.2, 1.4 Hz, 1H), 4.65-4.58 (m, 2H), 4.03 (s, 3H), 3.29-3.08 (m, 4H), 2.15-2.06 (m, 2H), 1.93-1.82 (m, 2H) |
| 107 | 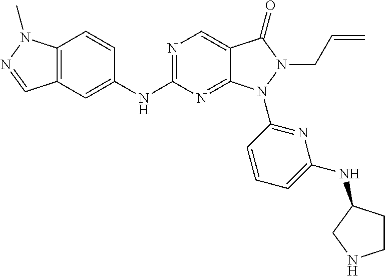 [SMILES: C=CCn1c(=O)c2cnc(Nc3ccc4c(cnn4C)c3)nc2n1-c1cccc(N[C@H]2CCNC2)n1] | 483.0 | 1H NMR (500 MHz, dmso) δ 10.32 (s, 1H), 8.86 (s, 1H), 8.72 (d, J = 22.2 Hz, 1H), 8.33 (s, 1H), 7.98 (s, 1H), 7.75 (s, 1H), 7.58 (t, J = 7.0 Hz, 1H), 7.19 (d, J = 4.7 Hz, 1H), 7.06 (d, J = 7.5 Hz, 1H), 6.52 (d, J = 8.2 Hz, 1H), 5.71 (ddt, J = 16.3, 10.8, 5.8 Hz, 1H), 5.07 (d, J = 10.2 Hz, 1H), 4.94 (d, J = 17.2 Hz, 1H), 4.61 (s, 2H), 4.39-4.33 (m, 2H), 4.03 (s, 3H), 3.44 (dt, J = 11.8, 5.9 Hz, 1H), 3.36-3.28 (m, 2H), 3.28-3.20 (m, 1H), 3.03 (dd, J = 11.3, 6.1 Hz, 1H), 2.21 (dq, J = 14.1, 7.2 Hz, 1H), 1.90 (dq, J = 13.2, 6.8 Hz, 1H) |
| 108 | 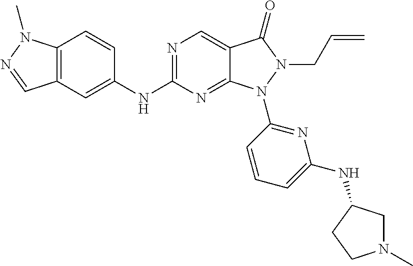 [SMILES: C=CCn1c(=O)c2cnc(Nc3ccc4c(cnn4C)c3)nc2n1-c1cccc(N[C@H]2CCN(C)C2)n1] | 497.2 | 1H NMR (500 MHz, MeOD) δ 8.82 (s, 1H), 8.25 (s, 1H), 7.90 (s, 1H), 7.69 (t, J = 7.9 Hz, 1H), 7.56 (dd, J = 9.0, 2.0 Hz, 1H), 7.49 (d, J = 9.0 Hz, 1H), 7.02 (d, J = 7.5 Hz, 1H), 6.58 (d, J = 8.3 Hz, 1H), 5.76 (ddt, J = 16.3, 10.3, 6.0 Hz, 1H), 5.11 (dd, J = 10.3, 1.1 Hz, 1H), 5.01 (dd, J = 17.1, 1.3 Hz, 1H), 4.74-4.63 (m, 2H), 4.47 (tt, J = 8.6, 4.7 Hz, 1H), 4.06 (s, 3H), 3.33 (d, J = 9.3 Hz, 1H), 3.14- 3.03 (m, 2H), 2.71 (s, 3H), 2.44 (td, J = 14.1, 8.2 Hz, 1H), 1.99 (dq, J = 13.3, 7.6 Hz, 1H), 1.94 (s, 1H) |
| 109 | 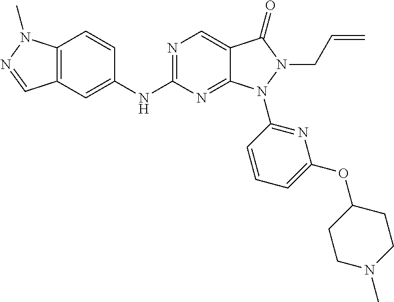 [SMILES: C=CCn1c(=O)c2cnc(Nc3ccc4c(cnn4C)c3)nc2n1-c1cccc(OC2CCN(C)CC2)n1] | 512.3 | 1H NMR (400 MHz, dmso) δ 10.42 (s, 1H), 9.36 (s, 1H), 8.89 (s, 1H), 8.26 (s, 1H), 8.05 (s, 1H), 8.02 (d, J = 2.7 Hz, 1H), 7.60 (s, 2H), 7.54 (d, J = 6.5 Hz, 1H), 6.84 (t, J = 8.3 Hz, 1H), 5.78-5.65 (m, 1H), 5.26 (s, 1H), 5.08 (d, J = 5.0 Hz, 1H), 4.94 (d, J = 17.1 Hz, 1H), 4.62 (s, 2H), 4.03 (s, 3H), 3.47 (s, 1H), 3.32 (s, 1H), 3.17 (s, 2H), 2.81 (dd, J = 10.5, 4.5 Hz, 3H), 2.25 (s, 1H), 2.11 (d, J = 14.1 Hz, 1H), 2.02 (d, J = 12.4 Hz, 1H), 1.78 (d, J = 13.7 Hz, 1H). |
| 110 | 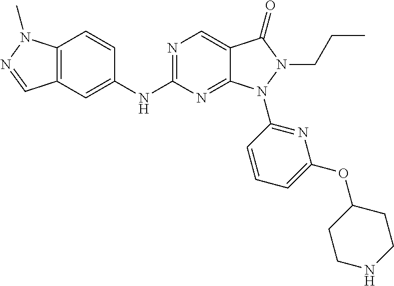 [SMILES: CCCn1c(=O)c2cnc(Nc3ccc4c(cnn4C)c3)nc2n1-c1cccc(OC2CCNCC2)n1] | 500.3 | 1H NMR (400 MHz, dmso) δ 10.38 (s, 1H), 8.87 (s, 1H), 8.47 (s, 2H), 8.27 (s, 1H), 8.09 (s, 1H), 8.02 (s, 1H), 7.60 (s, 3H), 6.87 (d, J = 8.2 Hz, 1H), 5.16 (s, 1H), 4.03 (s, 3H), 3.97 (s, 2H), 3.21 (s, 2H), 3.13 (s, 2H), 2.08 (s, 2H), 1.86 (s, 2H), 1.45 (q, J = 7.1 Hz, 2H), 0.71 (t, J = 7.3 Hz, 3H). |
| 111 | 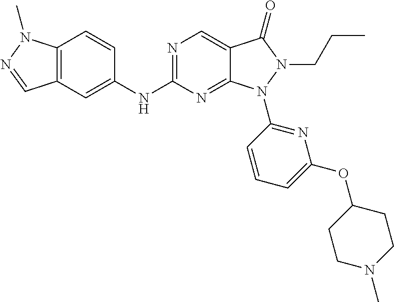 [SMILES: CCCn1c(=O)c2cnc(Nc3ccc4c(cnn4C)c3)nc2n1-c1cccc(OC2CCN(C)CC2)n1] | 514.3 | 1H NMR (400 MHz, dmso) δ 10.40 (s, 1H), 9.40 (s, 1H), 8.87 (s, 1H), 8.26 (s, 1H), 8.09 (s, 1H), 8.02 (s, 1H), 7.60 (s, 3H), 6.87 (s, 1H), 5.15 (d, J = 60.9 Hz, 1H), 4.03 (s, 3H), 3.98 (s, 2H), 3.46 (s, 1H), 3.34 (s, 1H), 3.16 (s, 2H), 2.79 (s, 3H), 2.22 (s, 1H), 2.07 (s, 2H), 1.79 (s, 1H), 1.45 (d, J = 7.0 Hz, 2H), 0.71 (t, J = 7.1 Hz, 3H) |
| 112 | 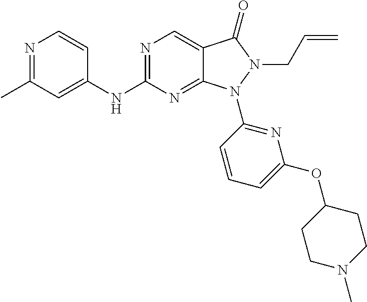 [SMILES: C=CCn1c(=O)c2cnc(Nc3ccnc(C)c3)nc2n1-c1cccc(OC2CCN(C)CC2)n1] | 473.0 | 1H NMR (400 MHz, DMSO) δ 11.57 (s, 1H), 9.62 (d, J = 34.3 Hz, 1H), 9.16 (s, 1H), 8.54 (d, J = 6.8 Hz, 1H), 8.18 (s, 1H), 8.09 (q, J = 8.0 Hz, 1H), 8.01- 7.84 (m, 1H), 7.53 (d, J = 7.6 Hz, 1H), 6.92 (dd, J = 8.3, 3.4 Hz, 1H), 5.73 (td, J = 11.4, 5.9 Hz, 1H), 5.26-5.08 (m, 2H), 4.94 (d, J = 17.2 Hz, 1H), 4.67 (d, J = 5.5 Hz, 2H), 3.16 (d, J = 10.7 Hz, 3H), 2.87-2.74 (m, 3H), 2.61 (s, 3H), 2.25 (d, J = 13.0 Hz, 1H), 2.15-1.95 (m, 2H), 1.90-1.70 (m, 1H). |
| 113 | 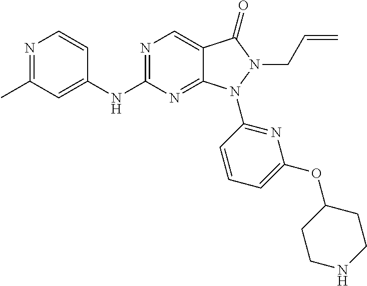 [SMILES: C=CCn1c(=O)c2cnc(Nc3ccnc(C)c3)nc2n1-c1cccc(OC2CCNCC2)n1] | 459.0 | 1H NMR (400 MHz, dmso) δ 11.53 (s, 1H), 9.16 (s, 1H), 8.57 (s, 1H), 8.53 (d, J = 6.8 Hz, 2H), 8.16 (s, 1H), 8.08 (t, J = 7.9 Hz, 1H), 7.90 (d, J = 6.9 Hz, 1H), 7.52 (d, J = 7.6 Hz, 1H), 6.93 (d, J = 8.2 Hz, 1H), 5.73 (ddt, J = 16.2, 10.6, 5.7 Hz, 1H), 5.18 (dq, J = 10.7, 3.2 Hz, 1H), 5.07 (d, J = 10.3 Hz, 1H), 4.93 (d, J = 17.1 Hz, 1H), 4.66 (d, J = 5.7 Hz, 2H), 3.24 (s, 3H), 3.14 (s, 2H), 2.60 (s, 3H), 2.10 (s, 2H), 1.86 (d, J = 9.8 Hz, 2H). |
| 114 | 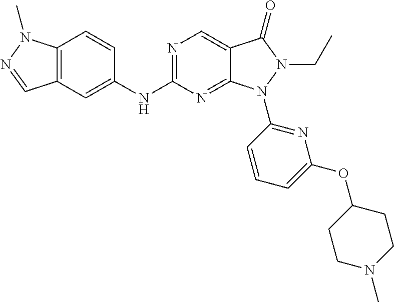 [SMILES: CCn1c(=O)c2cnc(Nc3ccc4c(cnn4C)c3)nc2n1-c1cccc(OC2CCN(C)CC2)n1] | 500.3 | 1H NMR (400 MHz, dmso) δ 10.40 (s, 1H), 9.37 (s, 1H), 8.86 (s, 1H), 8.26 (s, 1H), 8.09 (s, 1H), 8.02 (d, J = 3.1 Hz, 1H), 7.60 (s, 3H), 6.86 (t, J = 7.7 Hz, 1H), 5.23 (s, 1H), 5.07 (s, 1H), 4.03 (s, 3H), 3.46 (s, 1H), 3.31 (s, 1H), 3.15 (d, J = 10.6 Hz, 2H), 2.80 (dd, J = 11.5, 4.4 Hz, 3H), 2.23 (s, 1H), 2.10 (d, J = 13.9 Hz, 1H), 2.01 (d, J = 13.1 Hz, 1H), 1.77 (d, J = 12.5 Hz, 1H), 1.01 (d, J = 5.1 Hz, 3H). |
| 115 | 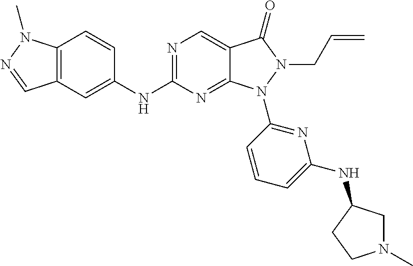 [SMILES: C=CCn1c(=O)c2cnc(Nc3ccc4c(cnn4C)c3)nc2n1-c1cccc(N[C@@H]2CCN(C)C2)n1] | 497.2 | 1H NMR (400 MHz, dmso) δ 10.38 (s, 1H), 9.77 (s, 1H), 8.86 (s, 1H), 8.35 (s, 1H), 7.99 (s, 1H), 7.77 (s, 1H), 7.59 (s, 2H), 7.36 (s, 0H), 7.19 (s, 1H), 7.07 (s, 1H), 6.58-6.47 (m, 1H), 5.70 (s, 1H), 5.07 (d, J = 10.1 Hz, 1H), 4.94 (d, J = 17.2 Hz, 1H), 4.61 (s, 2H), 4.42 (s, 1H), 4.03 (s, 3H), 3.87 (s, 1H), 3.66 (s, 1H), 3.46 (s, 1H), 3.29 (s, 1H), 3.14 (d, J = 40.6 Hz, 1H), 2.84 (dd, J = 16.4, 4.7 Hz, 3H), 2.25 (s, 1H), 1.97 (d, J = 51.4 Hz, 1H) |
| 116 | 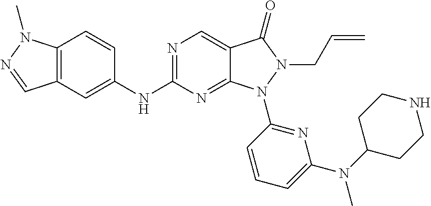 [SMILES: C=CCn1c(=O)c2cnc(Nc3ccc4c(cnn4C)c3)nc2n1-c1cccc(N(C)C2CCNCC2)n1] | 511.4 | 1H NMR (400 MHz, Methanol-d4) δ 8.82 (s, 1H), 8.55 (s, 1H), 8.29 (s, 1H), 7.91 (s, 1H), 7.77 (t, J = 8.0 Hz, 1H), 7.57 (dd, J = 9.0, 1.9 Hz, 1H), 7.49 (d, J = 9.0 Hz, 1H), 7.09 (d, J = 7.5 Hz, 1H), 6.67 (d, J = 8.5 Hz, 1H), 5.86-5.66 (m, 1H), 5.09 (d, J = 10.3 Hz, 1H), 4.97 (d, J = 17.1 Hz, 1H), 4.80-4.60 (m, 3H), 4.06 (s, 3H), 3.36 (s, 2H), 2.95 (s, 5H), 1.99-1.75 (m, 4H). One exchangeable proton not listed. |
| 117 | 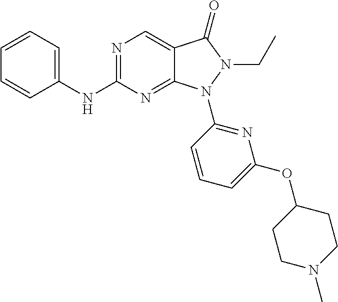 [SMILES: CCn1c(=O)c2cnc(Nc3ccccc3)nc2n1-c1cccc(OC2CCN(C)CC2)n1] | 446.0 | 1H NMR (400 MHz, dmso) δ 10.29 (s, 1H), 9.42 (s, 1H), 8.88 (s, 1H), 8.04 (q, J = 8.3 Hz, 1H), 7.74 (d, J = 7.9 Hz, 2H), 7.54 (d, J = 7.7 Hz, 1H), 7.33 (t, J = 7.8 Hz, 2H), 7.04 (t, J = 7.3 Hz, 1H), 6.85 (t, J = 7.4 Hz, 1H), 5.15 (d, J = 65.9 Hz, 1H), 4.07-3.91 (m, 2H), 3.48 (d, J = 11.8 Hz, 1H), 3.33 (d, J = 11.6 Hz, 1H), 3.15 (d, J = 11.6 Hz, 2H), 2.80 (dd, J = 13.5, 4.4 Hz, 3H), 2.25 (d, J = 12.5 Hz, 1H), 2.06 (dd, J = 36.7, 14.4 Hz, 2H), 1.78 (d, J = 12.5 Hz, 1H), 1.01 (q, J = 6.7 Hz, 3H). |
| 118 | 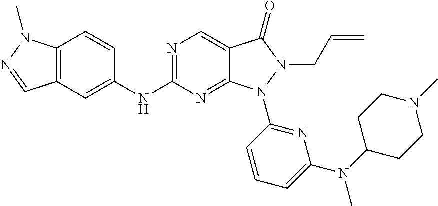 [SMILES: C=CCn1c(=O)c2cnc(Nc3ccc4c(cnn4C)c3)nc2n1-c1cccc(N(C)C2CCN(C)CC2)n1] | 525.3 | 1H NMR (400 MHz, Methanol-d4) δ 8.82 (s, 1H), 8.54 (s, 1H), 8.29 (s, 1H), 7.91 (s, 1H), 7.76 (t, J = 8.1 Hz, 1H), 7.58 (dd, J = 9.0, 1.9 Hz, 1H), 7.50 (d, J = 9.0 Hz, 1H), 7.08 (d, J = 7.5 Hz, 1H), 6.65 (d, J = 8.4 Hz, 1H), 5.83-5.67 (m, 1H), 5.08 (d, J = 10.1 Hz, 1H), 4.97 (d, J = 17.2 Hz, 1H), 4.75 (d, J = 5.9 Hz, 2H), 4.62 (s, 1H), 4.06 (s, 3H), 3.17 (d, J = 11.5 Hz, 2H), 2.93 (s, 3H), 2.61-2.36 (m, 5H), 2.06-1.86 (m, 2H), 1.75 (d, J = 10.4 Hz, 2H). |
| 119 | 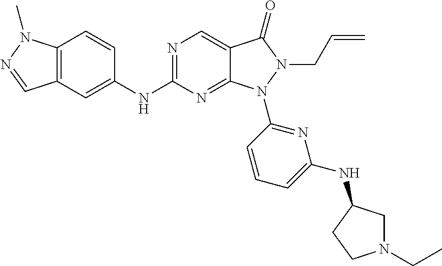 [SMILES: C=CCn1c(=O)c2cnc(Nc3ccc4c(cnn4C)c3)nc2n1-c1cccc(N[C@@H]2CCN(CC)C2)n1] | 511.3 | 1H NMR (400 MHz, dmso) δ 10.36 (s, 1H), 9.64 (s, 1H), 8.86 (s, 1H), 8.35 (s, 1H), 7.99 (s, 1H), 7.76 (s, 1H), 7.59 (s, 2H), 7.25 (d, J = 55.7 Hz, 1H), 7.06 (s, 1H), 6.53 (t, J = 7.4 Hz, 1H), 5.78-5.64 (m, 1H), 5.07 (d, J = 10.4 Hz, 1H), 4.94 (d, J = 17.2 Hz, 1H), 4.61 (s, 2H), 4.41 (s, 1H), 4.03 (s, 3H), 3.86 (s, 1H), 3.65 (s, 1H), 3.37 (d, J = 38.7 Hz, 1H), 3.25- 3.06 (m, 3H), 2.90 (s, 0H), 2.22 (s, 1H), 1.97 (d, J = 38.1 Hz, 1H), 1.17 (q, J = 7.0 Hz, 3H). |
| 120 | 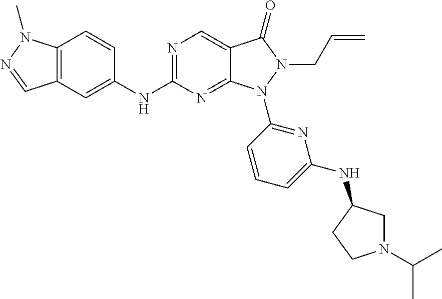 [SMILES: C=CCn1c(=O)c2cnc(Nc3ccc4c(cnn4C)c3)nc2n1-c1cccc(N[C@@H]2CCN(C(C)C)C2)n1] | 525.3 | 1H NMR (400 MHz, dmso) δ 10.36 (s, 1H), 9.63 (s, 1H), 8.87 (s, 1H), 8.36 (s, 1H), 7.99 (s, 1H), 7.78 (s, 1H), 7.59 (s, 2H), 7.25 (d, J = 44.8 Hz, 1H), 7.06 (s, 1H), 6.58-6.48 (m, 1H), 5.71 (d, J = 6.3 Hz, 1H), 5.07 (d, J = 10.2 Hz, 1H), 4.94 (d, J = 17.0 Hz, 1H), 4.60 (s, 2H), 4.39 (s, 1H), 4.03 (s, 3H), 3.79 (s, 1H), 3.60 (s, 1H), 3.41 (d, J = 6.6 Hz, 2H), 3.19 (s, 2H), 2.91 (s, 1H), 2.21 (s, 1H), 1.99 (s, 1H), 1.26-1.17 (m, 6H). |
| 121 | 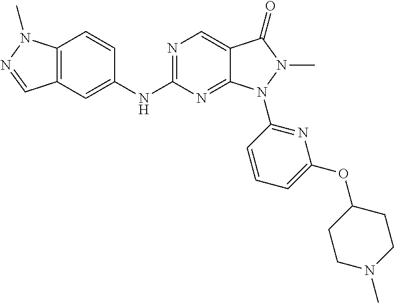 [SMILES: CN1CCC(Oc2cccc(-n3c4nc(Nc5ccc6c(cnn6C)c5)ncc4c(=O)n3C)n2)CC1] | 486.0 | 1H NMR (400 MHz, dmso) δ 10.39 (s, 1H), 9.33 (s, 1H), 8.87 (s, 1H), 8.26 (s, 1H), 8.09 (s, 1H), 8.03 (s, 1H), 7.61 (s, 3H), 6.83 (t, J = 8.8 Hz, 1H), 5.19 (d, J = 61.3 Hz, 1H), 4.04 (s, 3H), 3.43 (d, J = 7.2 Hz, 4H), 3.32 (s, 1H), 3.17 (s, 2H), 2.81 (dd, J = 11.3, 4.6 Hz, 3H), 2.25 (s, 1H), 2.05 (d, J = 36.8 Hz, 2H), 1.79 (s, 1H). |
| 122 | 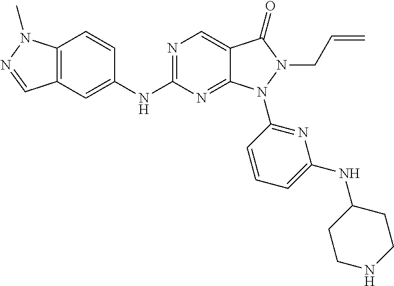 [SMILES: C=CCn1c(=O)c2cnc(Nc3ccc4c(cnn4C)c3)nc2n1-c1cccc(NC2CCNCC2)n1] | 497.2 | 1H NMR (400 MHz, dmso) δ 10.32 (s, 1H), 8.85 (s, 1H), 8.48 (s, 1H), 8.29 (s, 2H), 7.99 (s, 1H), 7.71 (s, 1H), 7.58 (d, J = 9.2 Hz, 2H), 7.00 (d, J = 7.6 Hz, 2H), 6.49 (d, J = 8.3 Hz, 1H), 5.77-5.65 (m, 1H), 5.06 (d, J = 10.3 Hz, 1H), 4.92 (d, J = 17.0 Hz, 1H), 4.62 (s, 2H), 4.02 (s, 3H), 3.89 (s, 1H), 3.26 (s, 2H), 3.03 (d, J = 10.5 Hz, 2H), 2.02 (d, J = 11.4 Hz, 2H), 1.59 (d, J = 10.7 Hz, 2H). |
| 123 | 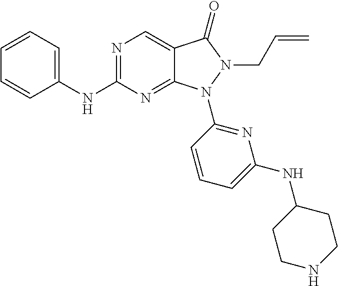 [SMILES: C=CCn1c(=O)c2cnc(Nc3ccccc3)nc2n1-c1cccc(NC2CCNCC2)n1] | 443.0 | 1H NMR (400 MHz, dmso) δ 10.23 (s, 1H), 8.86 (s, 1H), 8.47 (s, 1H), 8.27 (s, 1H), 7.76 (d, J = 7.9 Hz, 2H), 7.64 (t, J = 7.9 Hz, 1H), 7.30 (t, J = 7.8 Hz, 2H), 7.02 (s, 2H), 6.94 (d, J = 7.5 Hz, 1H), 6.48 (d, J = 8.3 Hz, 1H), 5.79-5.62 (m, 1H), 5.05 (d, J = 10.1 Hz, 1H), 4.92 (d, J = 17.0 Hz, 1H), 4.59 (s, 2H), 3.89 (s, 1H), 3.26 (s, 2H), 3.02 (d, J = 10.5 Hz, 2H), 2.02 (d, J = 11.0 Hz, 2H), 1.59 (d, J = 10.7 Hz, 2H). |
| 124 | 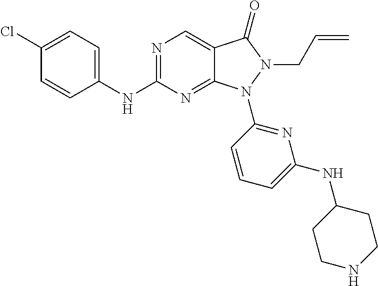 [SMILES: C=CCn1c(=O)c2cnc(Nc3ccc(Cl)cc3)nc2n1-c1cccc(NC2CCNCC2)n1] | 477.0 | 1H NMR (400 MHz, dmso) δ 10.37 (s, 1H), 8.89 (s, 1H), 8.48 (s, 1H), 8.29 (s, 1H), 7.81 (d, J = 8.8 Hz, 2H), 7.69 (t, J = 7.7 Hz, 1H), 7.37 (d, J = 8.8 Hz, 2H), 7.04 (d, J = 7.2 Hz, 1H), 6.92 (d, J = 7.5 Hz, 1H), 6.48 (d, J = 8.3 Hz, 1H), 5.70 (td, J = 10.5, 5.0 Hz, 1H), 5.05 (d, J = 10.3 Hz, 1H), 4.91 (d, J = 17.1 Hz, 1H), 4.61 (s, 2H), 3.88 (s, 1H), 3.26 (s, 2H), 3.02 (d, J = 10.3 Hz, 2H), 2.02 (d, J = 12.4 Hz, 2H), 1.59 (d, J = 10.9 Hz, 2H). |
| 125 | 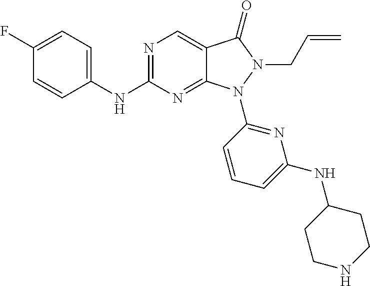 [SMILES: C=CCn1c(=O)c2cnc(Nc3ccc(F)cc3)nc2n1-c1cccc(NC2CCNCC2)n1] | 461.0 | 1H NMR (500 MHz, DMSO) δ 10.25 (s, 1H), 8.85 (s, 1H), 8.48 (s, 1H), 8.28 (s, 1H), 7.76 (dd, J = 8.7, 5.0 Hz, 2H), 7.65 (t, J = 7.9 Hz, 1H), 7.20-7.11 (m, 2H), 7.01 (d, J = 7.2 Hz, 1H), 6.92 (d, J = 7.5 Hz, 1H), 6.48 (d, J = 8.3 Hz, 1H), 5.76- 5.63 (m, 1H), 5.05 (dd, J = 10.3, 1.3 Hz, 1H), 4.92 (dd, J = 17.2, 1.4 Hz, 1H), 4.60 (s, 2H), 3.88 (s, 1H), 3.28 (d, J = 12.8 Hz, 2H), 3.03 (q, J = 11.3 Hz, 2H), 2.02 (d, J = 10.7 Hz, 2H), 1.67-1.53 (m, 2H). |
| 126 | 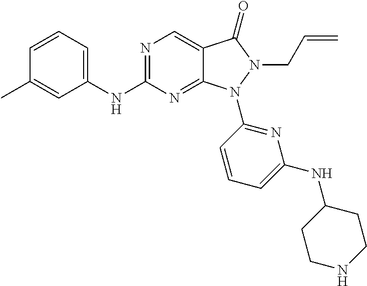 [SMILES: C=CCn1c(=O)c2cnc(Nc3cccc(C)c3)nc2n1-c1cccc(NC2CCNCC2)n1] | 457.3 | 1H NMR (400 MHz, DMSO-d6) δ 10.20 (s, 1H), 8.85 (s, 1H), 8.58-8.39 (m, 1H), 8.37-8.16 (m, 1H), 7.76 (s, 1H), 7.63 (t, J = 7.9 Hz, 1H), 7.44 (d, J = 7.8 Hz, 1H), 7.17 (t, J = 7.8 Hz, 1H), 7.04 (d, J = 6.3 Hz, 1H), 6.96 (d, J = 7.5 Hz, 1H), 6.84 (d, J = 7.4 Hz, 1H), 6.48 (d, J = 8.2 Hz, 1H), 5.70 (ddt, J = 16.2, 10.8, 5.7 Hz, 1H), 5.05 (d, J = 10.2 Hz, 1H), 4.91 (d, J = 17.2 Hz, 1H), 4.61 (d, J = 4.3 Hz, 2H), 3.89 (s, 1H), 3.28 (d, J = 11.8 Hz, 2H), 3.03 (d, J = 10.2 Hz, 2H), 2.28 (s, 3H), 2.02 (d, J = 11.5 Hz, 2H), 1.59 (q, J = 10.0 Hz, 2H). |
| 127 | 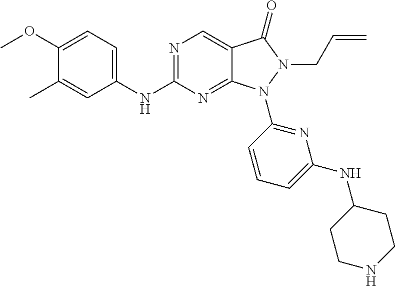 [SMILES: C=CCn1c(=O)c2cnc(Nc3ccc(OC)c(C)c3)nc2n1-c1cccc(NC2CCNCC2)n1] | 487.3 | 1H NMR (400 MHz, DMSO-d6) δ 10.10 (s, 1H), 8.80 (s, 1H), 8.57-8.42 (m, 1H), 8.38-8.19 (m, 1H), 7.69 (s, 1H), 7.62 (t, J = 7.9 Hz, 1H), 7.41 (dd, J = 8.9, 2.1 Hz, 1H), 7.02 (d, J = 6.2 Hz, 1H), 6.95 (d, J = 7.5 Hz, 1H), 6.87 (d, J = 8.9 Hz, 1H), 6.47 (d, J = 8.3 Hz, 1H), 5.69 (ddt, J = 16.3, 10.8, 5.6 Hz, 1H), 5.04 (d, J = 10.4 Hz, 1H), 4.90 (d, J = 17.5 Hz, 1H), 4.60 (s, 2H), 3.94-3.85 (m, 1H), 3.76 (s, 3H), 3.35-3.21 (m, 2H), 3.10-2.96 (m, 2H), 2.13 (s, 3H), 2.02 (d, J = 11.0 Hz, 2H), 1.59 (q, J = 10.2 Hz, 2H). |
| 128 | 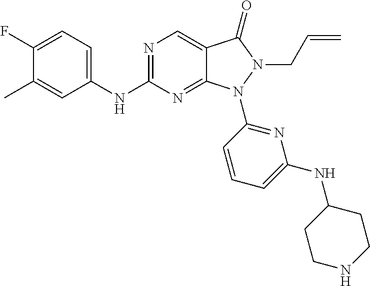 [SMILES: C=CCn1c(=O)c2cnc(Nc3ccc(F)c(C)c3)nc2n1-c1cccc(NC2CCNCC2)n1] | 475.0 | 1H NMR (400 MHz, DMSO-d6) δ 10.23 (s, 1H), 8.85 (s, 1H), 8.58-8.47 (m, 1H), 8.43-8.23 (m, 1H), 7.89- 7.75 (m, 1H), 7.63 (t, J = 7.9 Hz, 1H), 7.52-7.39 (m, 1H), 7.08 (t, J = 9.2 Hz, 1H), 7.05-6.98 (m, 1H), 6.94 (d, J = 7.5 Hz, 1H), 6.48 (d, J = 8.3 Hz, 1H), 5.77-5.60 (m, 1H), 5.05 (d, J = 10.4 Hz, 1H), 4.90 (d, J = 17.2 Hz, 1H), 4.66-4.51 (m, 2H), 3.96-3.82 (m, 1H), 3.35-3.22 (m, 2H), 3.12-2.95 (m, 2H), 2.21 (s, 3H), 2.05-1.94 (m, 2H), 1.68-1.50 (m, 2H). |
| 129 | 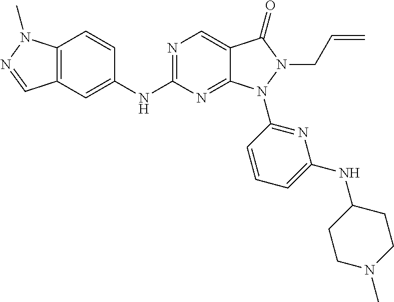 [SMILES: C=CCn1c(=O)c2cnc(Nc3ccc4c(cnn4C)c3)nc2n1-c1cccc(NC2CCN(C)CC2)n1] | 511.0 | 1H NMR (400 MHz, dmso) δ 10.34 (s, 1H), 9.21 (s, 1H), 8.85 (s, 1H), 8.34 (s, 1H), 7.99 (s, 1H), 7.70 (s, 1H), 7.59 (s, 2H), 7.01 (d, J = 7.3 Hz, 2H), 6.47 (d, J = 8.3 Hz, 1H), 5.70 (s, 1H), 5.06 (d, J = 10.0 Hz, 1H), 4.92 (d, J = 17.0 Hz, 1H), 4.62 (s, 2H), 4.03 (s, 3H), 3.85 (s, 1H), 3.43 (d, J = 11.5 Hz, 2H), 3.29 (s, 1H), 3.09 (d, J = 12.3 Hz, 2H), 2.77 (d, J = 4.5 Hz, 3H), 2.10 (d, J = 13.2 Hz, 1H), 1.94 (s, 1H), 1.58 (d, J = 13.7 Hz, 1H). |
| 130 | 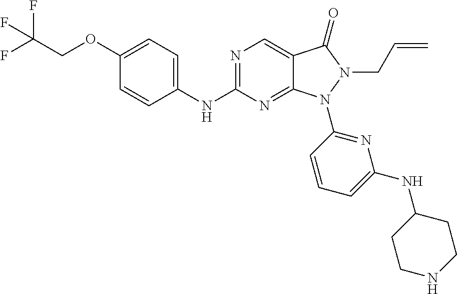 [SMILES: C=CCn1c(=O)c2cnc(Nc3ccc(OCC(F)(F)F)cc3)nc2n1-c1cccc(NC2CCNCC2)n1] | 541.0 | 1H NMR (400 MHz, dmso) δ 10.18 (s, 1H), 8.83 (s, 1H), 7.69 (s, 2H), 7.61 (s, 1H), 7.02 (d, J = 8.9 Hz, 2H), 6.89 (d, J = 7.5 Hz, 1H), 6.85 (d, J = 7.6 Hz, 1H), 6.43 (d, J = 8.2 Hz, 1H), 5.70 (dd, J = 16.9, 10.5 Hz, 1H), 5.04 (d, J = 10.4 Hz, 1H), 4.91 (d, J = 17.2 Hz, 1H), 4.72 (q, J = 8.8 Hz, 2H), 4.61 (s, 2H), 3.72 (s, 1H), 3.02 (d, J = 12.0 Hz, 2H), 2.63 (t, J = 11.9 Hz, 3H), 1.92-1.81 (m, 2H), 1.36 (d, J = 10.7 Hz, 2H). |
| 131 | 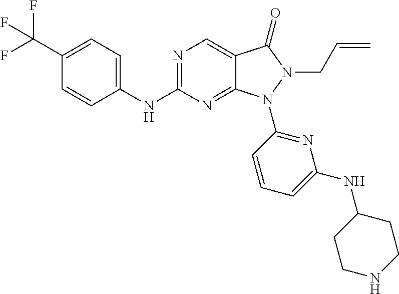 [SMILES: C=CCn1c(=O)c2cnc(Nc3ccc(C(F)(F)F)cc3)nc2n1-c1cccc(NC2CCNCC2)n1] | 511.0 | 1H NMR (400 MHz, DMSO-d6) δ 10.61 (s, 1H), 8.94 (s, 1H), 8.56-8.44 (m, 1H), 8.37-8.20 (m, 1H), 8.01 (d, J = 8.5 Hz, 2H), 7.72 (t, J = 7.8 Hz, 1H), 7.67 (d, J = 8.8 Hz, 2H), 7.06 (d, J = 7.4 Hz, 1H), 6.94 (d, J = 7.5 Hz, 1H), 6.50 (d, J = 8.3 Hz, 1H), 5.71 (ddd, J = 16.0, 10.6, 5.5 Hz, 1H), 5.06 (d, J = 10.1 Hz, 1H), 4.92 (d, J = 17.0 Hz, 1H), 4.61 (d, J = 5.6 Hz, 2H), 3.94-3.84 (m, 1H), 3.33-3.23 (m, 2H), 3.08-2.94 (m, 2H), 2.08-1.96 (m, 2H), 1.67-1.53 (m, 2H). |
| 132 | 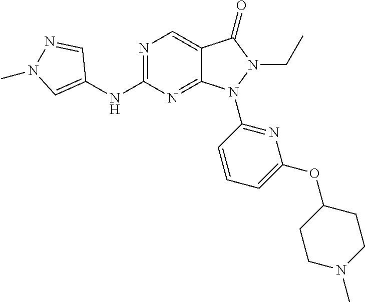 [SMILES: CCn1c(=O)c2cnc(Nc3cnn(C)c3)nc2n1-c1cccc(OC2CCN(C)CC2)n1] | 450.0 | 1H NMR (400 MHz, Methanol-d4) δ 8.92-8.72 (m, 1H), 8.54 (s, 1H), 8.13- 7.78 (m, 2H), 7.66-7.52 (m, 1H), 7.50 (d, J = 7.5 Hz, 1H), 6.95-6.76 (m, 1H), 5.27-5.09 (m, 1H), 4.09 (q, J = 7.0 Hz, 2H), 3.86 (s, 3H), 3.14-3.02 (m, 2H), 2.95-2.79 (m, 2H), 2.61 (s, 3H), 2.22- 2.07 (m, 2H), 2.06-1.93 (m, 2H), 1.12 (t, J = 7.0 Hz, 3H). |
| 133 | 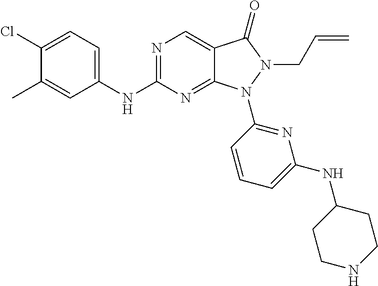 [SMILES: C=CCn1c(=O)c2cnc(Nc3ccc(Cl)c(C)c3)nc2n1-c1cccc(NC2CCNCC2)n1] | 491.0 | 1H NMR (400 MHz, DMSO-d6) δ 10.32 (s, 1H), 8.87 (s, 1H), 8.63-8.48 (m, 1H), 8.39-8.24 (m, 1H), 7.91 (s, 1H), 7.66 (t, J = 7.9 Hz, 1H), 7.51 (d, J = 7.9 Hz, 1H), 7.33 (d, J = 8.7 Hz, 1H), 7.11-7.01 (m, 1H), 6.94 (d, J = 7.5 Hz, 1H), 6.50 (d, J = 8.3 Hz, 1H), 5.70 (ddt, J = 16.1, 10.5, 5.7 Hz, 1H), 5.05 (d, J = 10.3 Hz, 1H), 4.91 (d, J = 17.4 Hz, 1H), 4.61 (d, J = 5.1 Hz, 2H), 3.97-3.83 (m, 1H), 3.34-3.20 (m, 2H), 3.11-2.91 (m, 2H), 2.30 (s, 3H), 2.09-1.94 (m, 2H), 1.66-1.51 (m, 2H). |
| 134 | 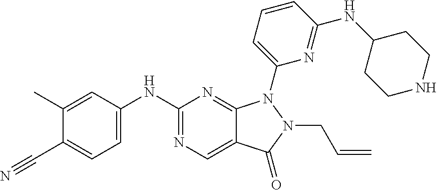 [SMILES: C=CCn1c(=O)c2cnc(Nc3ccc(C#N)c(C)c3)nc2n1-c1cccc(NC2CCNCC2)n1] | 482.1 | 1H NMR (500 MHz, DMSO) δ 10.63 (s, 1H), 8.95 (s, 1H), 8.58-8.38 (m, 1H), 8.26 (q, J = 10.8 Hz, 1H), 8.06 (s, 1H), 7.72-7.63 (m, 3H), 7.07 (d, J = 7.3 Hz, 1H), 6.96 (d, J = 7.5 Hz, 1H), 6.53 (d, J = 8.3 Hz, 1H), 5.72 (ddt, J = 17.2, 10.3, 5.7 Hz, 1H), 5.07 (dd, J = 10.3, 1.5 Hz, 1H), 4.92 (dq, J = 17.1, 1.5 Hz, 1H), 4.63 (d, J = 5.7 Hz, 2H), 3.97-3.84 (m, 1H), 3.28 (d, J = 13.7 Hz, 2H), 3.03 (qd, J = 11.3, 3.0 Hz, 2H), 2.44 (s, 3H), 2.09- 1.94 (m, 2H), 1.66-1.54 (m, 2H) |
| 135 | 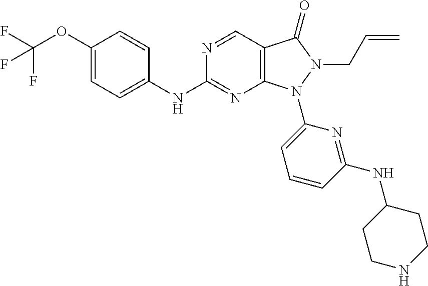 [SMILES: C=CCn1c(=O)c2cnc(Nc3ccc(OC(F)(F)F)cc3)nc2n1-c1cccc(NC2CCNCC2)n1] | 527.0 | 1H NMR (400 MHz, dmso) δ 10.40 (s, 1H), 8.89 (s, 1H), 7.88 (d, J = 8.6 Hz, 2H), 7.61 (s, 1H), 7.32 (d, J = 8.6 Hz, 2H), 6.90-6.78 (m, 2H), 6.44 (d, J = 8.2 Hz, 1H), 5.70 (s, 1H), 5.05 (d, J = 10.3 Hz, 1H), 4.92 (d, J = 17.1 Hz, 1H), 4.61 (s, 2H), 3.66 (s, 1H), 2.94 (d, J = 12.1 Hz, 2H), 2.54 (s, 1H), 1.88 (s, 1H), 1.81 (d, J = 10.8 Hz, 2H), 1.35-1.21 (m, 3H). |
| 136 | 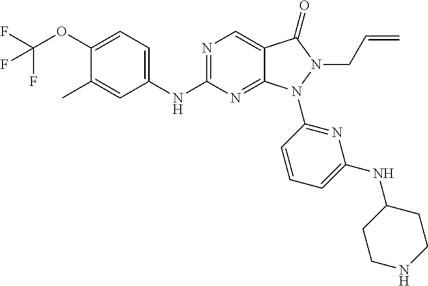 [SMILES: C=CCn1c(=O)c2cnc(Nc3ccc(OC(F)(F)F)c(C)c3)nc2n1-c1cccc(NC2CCNCC2)n1] | 541.0 | 1H NMR (400 MHz, dmso) δ 10.37 (s, 1H), 8.89 (s, 1H), 8.47 (s, 1H), 8.28 (s, 1H), 7.94 (s, 1H), 7.65 (t, J = 7.9 Hz, 1H), 7.56 (d, J = 8.0 Hz, 1H), 7.25 (d, J = 8.6 Hz, 1H), 7.05 (d, J = 7.3 Hz, 1H), 6.95 (d, J = 7.5 Hz, 1H), 6.50 (d, J = 8.3 Hz, 1H), 5.70 (d, J = 6.5 Hz, 1H), 5.05 (d, J = 10.1 Hz, 1H), 4.91 (d, J = 17.1 Hz, 1H), 4.61 (s, 2H), 3.90 (s, 1H), 3.26 (s, 2H), 3.03 (d, J = 10.2 Hz, 2H), 2.25 (s, 3H), 2.02 (d, J = 11.4 Hz, 2H), 1.59 (d, J = 11.0 Hz, 2H). |
| 137 | 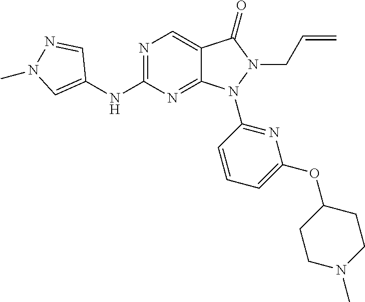 [SMILES: C=CCn1c(=O)c2cnc(Nc3cnn(C)c3)nc2n1-c1cccc(OC2CCN(C)CC2)n1] | 462.0 | 1H NMR (400 MHz, DMSO-d6) δ 10.45-10.02 (m, 1H), 8.92-8.75 (m, 1H), 8.00 (t, J = 7.9 Hz, 1H), 7.92-7.79 (m, 1H), 7.57-7.47 (m, 1H), 7.42 (d, J = 7.7 Hz, 1H), 6.81 (d, J = 7.9 Hz, 1H), 5.79-5.63 (m, 1H), 5.05 (d, J = 9.6 Hz, 1H), 4.98-4.86 (m, 2H), 4.62-4.50 (m, 2H), 3.80 (s, 3H), 2.71-2.58 (m, 2H), 2.30-2.14 (m, 5H), 2.03-1.86 (m, 2H), 1.77-1.63 (m, 2H). |
| 138 | 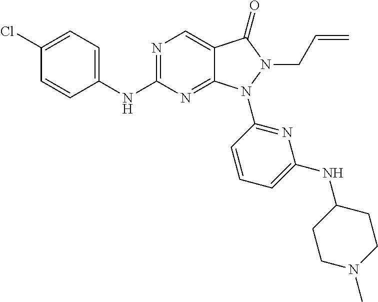 [SMILES: C=CCn1c(=O)c2cnc(Nc3ccc(Cl)cc3)nc2n1-c1cccc(NC2CCN(C)CC2)n1] | 491.0 | 1H NMR (400 MHz, Methanol-d4) δ 8.83 (s, 1H), 7.71 (d, J = 8.9 Hz, 2H), 7.68-7.60 (m, 1H), 7.27 (d, J = 9.0 Hz, 2H), 7.05-6.90 (m, 1H), 6.64-6.47 (m, 1H), 5.85-5.68 (m, 1H), 5.10 (d, J = 10.2 Hz, 1H), 4.99 (d, J = 17.1 Hz, 1H), 4.69 (d, J = 5.8 Hz, 2H), 4.22- 3.91 (m, 1H), 3.62-3.36 (m, 2H), 3.27- 3.08 (m, 2H), 2.92-2.83 (m, 3H), 2.40-2.12 (m, 2H), 2.09-1.57 (m, 2H). 3 exchangeable protons not listed |
| 139 | 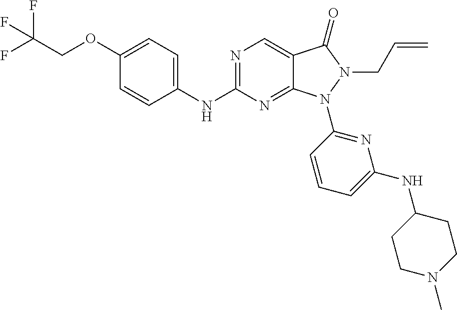 [SMILES: C=CCn1c(=O)c2cnc(Nc3ccc(OCC(F)(F)F)cc3)nc2n1-c1cccc(NC2CCN(C)CC2)n1] | 555.0 | 1H NMR (400 MHz, Methanol-d4) δ 8.80 (s, 1H), 7.71-7.58 (m, 3H), 7.04- 6.91 (m, 3H), 6.63-6.46 (m, 1H), 5.82- 5.68 (m, 1H), 5.10 (d, J = 10.0 Hz, 1H), 4.99 (d, J = 17.1 Hz, 1H), 4.69 (d, J = 5.8 Hz, 2H), 4.51 (q, J = 8.6 Hz, 2H), 4.19-3.94 (m, 1H), 3.61-3.38 (m, 2H), 3.25-3.09 (m, 2H), 2.92-2.85 (m, 3H), 2.38-2.12 (m, 2H), 1.70 (s, 2H). 3 exchangeable protons not listed |
| 140 | 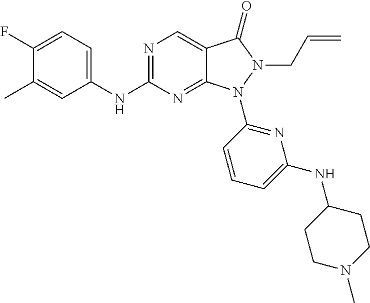 [SMILES: C=CCn1c(=O)c2cnc(Nc3ccc(F)c(C)c3)nc2n1-c1cccc(NC2CCN(C)CC2)n1] | 489.0 | 1H NMR (400 MHz, Methanol-d4) δ 8.80 (s, 1H), 7.77-7.57 (m, 2H), 7.46- 7.35 (m, 1H), 7.06-6.89 (m, 2H), 6.56 (dd, J = 33.7, 8.2 Hz, 1H), 5.83-5.68 (m, 1H), 5.10 (d, J = 10.2 Hz, 1H), 4.99 (d, J = 17.1 Hz, 1H), 4.69 (d, J = 5.9 Hz, 2H), 4.20-3.92 (m, 1H), 3.59-3.37 (m, 2H), 3.26-3.09 (m, 2H), 2.87 (d, J = 4.7 Hz, 3H), 2.26 (d, J = 18.1 Hz, 5H), 2.09-1.61 (m, 2H). 3 exchangeable protons not listed |
| 141 | 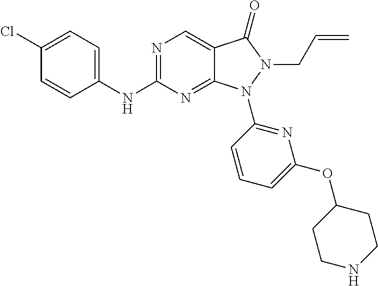 [SMILES: C=CCn1c(=O)c2cnc(Nc3ccc(Cl)cc3)nc2n1-c1cccc(OC2CCNCC2)n1] | 478.0 | 1H NMR (400 MHz, dmso) δ 10.43 (s, 1H), 8.91 (s, 1H), 7.99 (t, J = 8.0 Hz, 1H), 7.79 (d, J = 8.8 Hz, 2H), 7.39 (t, J = 7.8 Hz, 3H), 6.79 (d, J = 8.2 Hz, 1H), 5.71 (ddt, J = 16.3, 10.8, 5.8 Hz, 1H), 5.05 (d, J = 10.3 Hz, 1H), 5.03-4.95 (m, 1H), 4.92 (d, J = 17.3 Hz, 1H), 4.61 (d, J = 5.8 Hz, 2H), 2.94 (dt, J = 12.5, 4.4 Hz, 2H), 2.61-2.53 (m, 2H), 2.24 (s, 1H), 1.98-1.82 (m, 2H), 1.57-1.41 (m, 2H). |
| 142 | 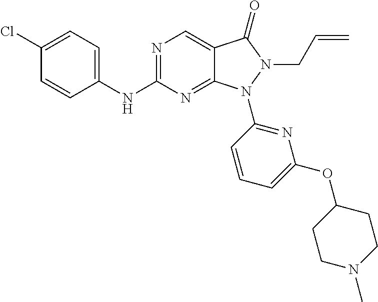 [SMILES: C=CCn1c(=O)c2cnc(Nc3ccc(Cl)cc3)nc2n1-c1cccc(OC2CCN(C)CC2)n1] | 492.0 | 1H NMR (400 MHz, dmso) δ 10.43 (s, 1H), 8.91 (s, 1H), 7.99 (t, J = 7.9 Hz, 1H), 7.84-7.71 (m, 2H), 7.40 (t, J = 8.9 Hz, 3H), 6.79 (d, J = 8.2 Hz, 1H), 5.70 (ddt, J = 16.3, 10.8, 5.1 Hz, 1H), 5.05 (d, J = 10.3 Hz, 1H), 4.99-4.86 (m, 2H), 4.60 (d, J = 5.8 Hz, 2H), 2.65- 2.55 (m, 2H), 2.24-2.07 (m, 5H), 1.99- 1.87 (m, 2H), 1.76-1.58 (m, 2H) |
| 143 | 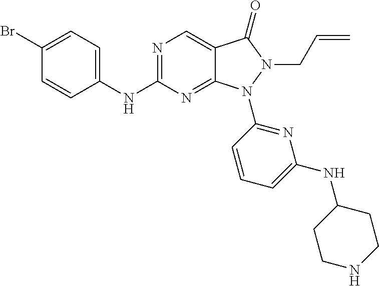 [SMILES: C=CCn1c(=O)c2cnc(Nc3ccc(Br)cc3)nc2n1-c1cccc(NC2CCNCC2)n1] | 521.0 | 1H NMR (400 MHz, dmso) δ 10.35 (s, 1H), 8.88 (s, 1H), 7.77 (d, J = 8.5 Hz, 2H), 7.62 (s, 1H), 7.48 (d, J = 8.6 Hz, 2H), 6.83 (dd, J = 13.0, 7.7 Hz, 2H), 6.44 (d, J = 8.3 Hz, 1H), 5.71 (d, J = 6.3 Hz, 1H), 5.05 (d, J = 10.2 Hz, 1H), 4.91 (d, J = 17.1 Hz, 1H), 4.60 (s, 2H), 3.66 (s, 1H), 3.57 (s, 1H), 2.93 (d, J = 11.9 Hz, 2H), 2.53 (s, 1H), 1.80 (d, J = 11.2 Hz, 2H), 1.35-1.20 (m, 3H). |
| 144 | 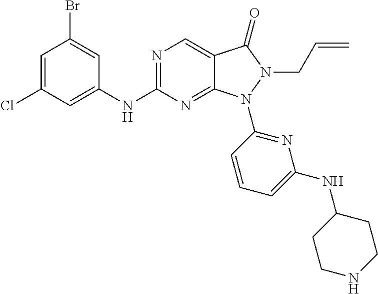 [SMILES: C=CCn1c(=O)c2cnc(Nc3cc(Cl)cc(Br)c3)nc2n1-c1cccc(NC2CCNCC2)n1] | 557.0 | 1H NMR (400 MHz, dmso) δ 10.58 (s, 1H), 8.94 (s, 1H), 8.47 (s, 1H), 8.26 (s, 1H), 8.09 (s, 1H), 7.94 (s, 1H), 7.69 (t, J = 7.8 Hz, 1H), 7.32 (s, 1H), 7.07 (d, J = 7.2 Hz, 1H), 6.93 (d, J = 7.4 Hz, 1H), 6.51 (d, J = 8.3 Hz, 1H), 5.71 (dd, J = 17.0, 10.5 Hz, 1H), 5.05 (d, J = 10.0 Hz, 1H), 4.91 (d, J = 16.8 Hz, 1H), 4.64 (s, 2H), 3.89 (s, 1H), 3.26 (s, 2H), 3.04 (s, 2H), 2.02 (d, J = 11.7 Hz, 2H), 1.59 (d, J = 10.7 Hz, 2H). |
| 145 | 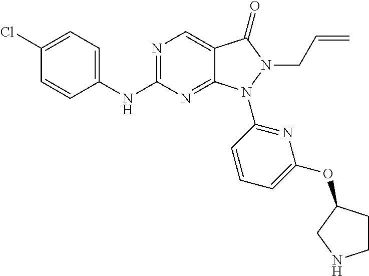 [SMILES: C=CCn1c(=O)c2cnc(Nc3ccc(Cl)cc3)nc2n1-c1cccc(O[C@H]2CCNC2)n1] | 464.0 | 1H NMR (400 MHz, dmso) δ 10.47 (s, 1H), 9.04 (s, 1H), 8.93 (s, 1H), 8.88 (s, 1H), 8.08 (s, 1H), 7.79 (d, J = 8.8 Hz, 2H), 7.50 (d, J = 7.7 Hz, 1H), 7.40 (d, J = 8.7 Hz, 2H), 6.84 (d, J = 8.1 Hz, 1H), 5.79-5.63 (m, 1H), 5.54 (s, 1H), 5.06 (d, J = 10.3 Hz, 1H), 4.95 (d, J = 17.1 Hz, 1H), 4.62 (d, J = 5.1 Hz, 2H), 3.50 (d, J = 6.6 Hz, 1H), 3.41 (s, 1H), 3.31 (s, 2H), 2.32-2.22 (m, 1H), 2.17 (s, 1H) |
| 146 | 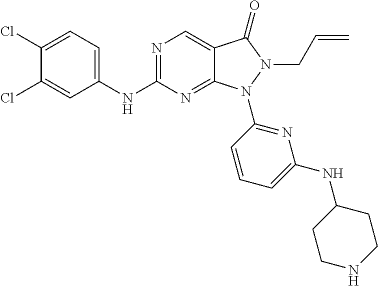 [SMILES: C=CCn1c(=O)c2cnc(Nc3ccc(Cl)c(Cl)c3)nc2n1-c1cccc(NC2CCNCC2)n1] | 511.0 | 1H NMR (400 MHz, DMSO-d6) δ 10.55 (s, 1H), 8.92 (s, 1H), 8.52 (s, 1H), 8.44-8.17 (m, 2H), 7.68 (t, J = 7.9 Hz, 1H), 7.56 (s, 2H), 7.07 (d, J = 7.4 Hz, 1H), 6.93 (d, J = 7.4 Hz, 1H), 6.51 (d, J = 8.3 Hz, 1H), 5.71 (ddt, J = 15.9, 10.7, 5.6 Hz, 1H), 5.05 (d, J = 9.3 Hz, 1H), 4.91 (d, J = 17.2 Hz, 1H), 4.62 (d, J = 5.0 Hz, 2H), 4.04-3.78 (m, 1H), 3.32- 3.25 (m, 2H), 3.12-2.92 (m, 2H), 2.10- 1.95 (m, 2H), 1.71-1.49 (m, 2H). |
| 147 | 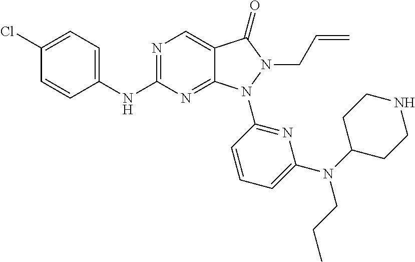 [SMILES: C=CCn1c(=O)c2cnc(Nc3ccc(Cl)cc3)nc2n1-c1cccc(N(CCC)C2CCNCC2)n1] | 519.0 | 1H NMR (400 MHz, dmso) δ 10.38 (s, 1H), 8.89 (s, 1H), 8.49 (s, 1H), 8.17 (s, 1H), 7.81 (d, J = 8.7 Hz, 3H), 7.37 (d, J = 8.8 Hz, 2H), 7.01 (d, J = 7.6 Hz, 1H), 6.66 (d, J = 8.5 Hz, 1H), 5.71 (dd, J = 16.9, 10.5 Hz, 1H), 5.05 (d, J = 10.2 Hz, 1H), 4.90 (d, J = 17.5 Hz, 1H), 4.62 (s, 2H), 4.47 (s, 1H), 3.37 (d, J = 12.0 Hz, 2H), 3.22 (s, 2H), 3.01 (d, J = 11.3 Hz, 2H), 1.95-1.77 (m, 4H), 1.51 (s, 2H), 0.89 (t, J = 7.3 Hz, 3H) |
| 148 | 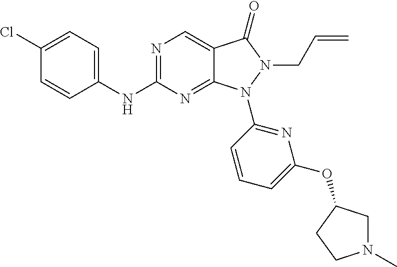 [SMILES: C=CCn1c(=O)c2cnc(Nc3ccc(Cl)cc3)nc2n1-c1cccc(O[C@H]2CCN(C)C2)n1] | 478.0 | 1H NMR (400 MHz, dmso) δ 10.47 (s, 1H), 9.95 (d, J = 102.8 Hz, 1H), 8.93 (s, 1H), 8.09 (s, 1H), 7.79 (d, J = 8.8 Hz, 2H), 7.51 (d, J = 7.7 Hz, 1H), 7.40 (d, J = 8.8 Hz, 2H), 6.93-6.80 (m, 1H), 5.71 (d, J = 6.2 Hz, 1H), 5.52 (d, J = 30.9 Hz, 1H), 5.07 (d, J = 10.3 Hz, 1H), 4.95 (d, J = 17.2 Hz, 1H), 4.62 (s, 2H), 3.98 (s, 1H), 3.75 (s, 1H), 3.40 (s, 1H), 3.27 (s, 1H), 3.13 (s, 1H), 2.88 (dd, J = 25.5, 4.3 Hz, 3H), 2.61 (s, 1H), 2.22 (d, J = 84.0 Hz, 1H) |
| 149 | 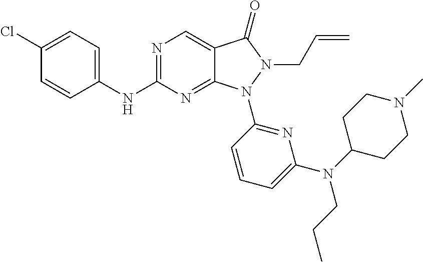 [SMILES: C=CCn1c(=O)c2cnc(Nc3ccc(Cl)cc3)nc2n1-c1cccc(N(CCC)C2CCN(C)CC2)n1] | 533.3 | 1H NMR (400 MHz, dmso) δ 10.38 (s, 1H), 9.27 (s, 1H),8.89 (s, 1H), 7.81 (d, J = 8.8 Hz, 3H), 7.38 (d, J = 8.9 Hz,2H), 7.03 (d, J = 7.6 Hz, 1H), 6.63 (d, J = 8.5 Hz, 1H), 5.70(dd, J = 16.9, 10.5 Hz, 1H), 5.05 (d, J = 10.0 Hz, 1H), 4.89(d, J = 17.4 Hz, 1H), 4.64 (s, 2H), 4.53 (s, 1H), 3.49 (d, J = 11.7 Hz, 2H), 3.25- 3.06 (m, 4H), 2.81 (d, J = 4.4 Hz, 3H), 1.99-1.79 (m, 4H), 1.53 (s, 2H), 0.90 (t, J = 7.2 Hz, 3H). |
| 150 | 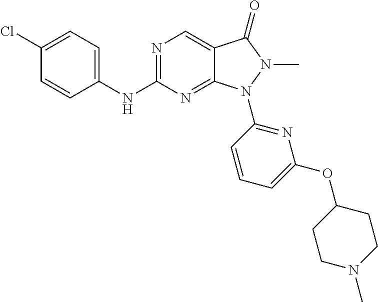 [SMILES: CN1CCC(Oc2cccc(-n3c4nc(Nc5ccc(Cl)cc5)ncc4c(=O)n3C)n2)CC1] | 466.0 | 1H NMR (500 MHz, DMSO) δ 10.41 (s, 1H), 9.47 (d, J = 30.4 Hz, 1H), 8.91 (s, 1H), 8.09 (dt, J = 10.9, 7.9 Hz, 1H), 7.86- 7.73 (m, 2H), 7.54 (dd, J = 7.7, 3.2 Hz, 1H), 7.46-7.36 (m, 2H), 6.84 (dd, J = 11.6, 8.1 Hz, 1H), 5.29-5.06 (m, 1H), 3.49 (d, J = 12.6 Hz, 1H), 3.43 (d, J = 9.2 Hz, 3H), 3.35 (d, J = 12.2 Hz, 1H), 3.17 (dt, J = 14.4, 10.3 Hz, 2H), 2.82 (dd, J = 16.3, 4.6 Hz, 3H), 2.32-2.22 (m, 1H), 2.13 (d, J = 15.5 Hz, 1H), 2.02 (t, J = 14.0 Hz, 1H), 1.85-1.73 (m, 1H). |
| 151 | 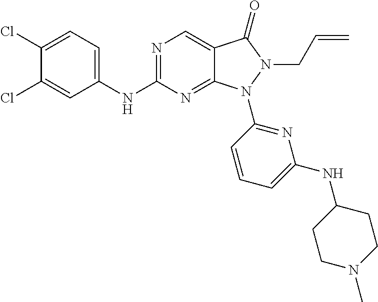 [SMILES: C=CCn1c(=O)c2cnc(Nc3ccc(Cl)c(Cl)c3)nc2n1-c1cccc(NC2CCN(C)CC2)n1] | 526.0 | 1H NMR (400 MHz, DMSO-d6) δ 10.56 (s, 1H), 9.43-9.16 (m, 1H), 8.92 (s, 1H), 8.38 (s, 1H), 7.78-7.60 (m, 1H), 7.62-7.48 (m, 2H), 7.05 (d, J = 7.3 Hz, 1H), 7.00-6.90 (m, 1H), 6.61- 6.44 (m, 1H), 5.70 (dq, J = 16.2, 5.6 Hz, 1H), 5.06 (d, J = 10.3 Hz, 1H), 4.91 (d, J = 17.1 Hz, 1H), 4.70-4.57 (m, 2H), 4.07-3.74 (m, 1H), 3.47-3.42 (m, 2H), 3.23-3.01 (m, 2H), 2.81-2.72 (m, 3H), 2.16-1.88 (m, 2H), 1.65- 1.48 (m, 2H). |
| 152 | 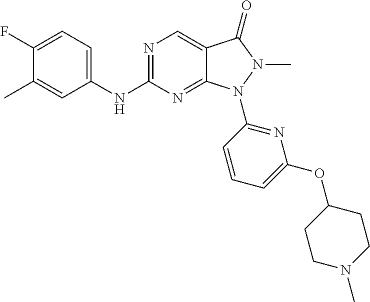 [SMILES: Cc1cc(Nc2ncc3c(=O)n(C)n(-c4cccc(OC5CCN(C)CC5)n4)c3n2)ccc1F] | 464.3 | H NMR (500 MHz, DMSO) δ 10.27 (s, 1H), 9.44 (d, J = 27.2 Hz, 1H), 8.87 (s, 1H), 8.02 (dt, J = 11.9, 8.0 Hz, 1H), 7.74 (s, 1H), 7.56 (dd, J = 7.8, 1.5 Hz, 1H), 7.47 (dt, J = 7.8, 3.6 Hz, 1H), 7.12 (t, J = 9.1 Hz, 1H), 6.84 (dd, J = 11.3, 8.1 Hz, 1H), 5.30-5.07 (m, 1H), 3.49 (d, J = 12.5 Hz, 1H), 3.43 (d, J = 8.6 Hz, 3H), 3.35 (d, J = 12.1 Hz, 1H), 3.18 (p, J = 11.5 Hz, 2H), 2.82 (dd, J = 15.9, 4.6 Hz, 3H), 2.27 (d, J = 13.2 Hz, 1H), 2.24 (t, J = 2.4 Hz, 3H), 2.13 (d, J = 15.0 Hz, 1H), 2.02 (dd, J = 15.7, 12.2 Hz, 1H), 1.85- 1.73 (m, 1H). Mixture of two conformers |
| 153 | 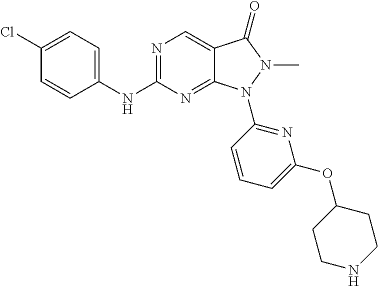 [SMILES: Cn1c(=O)c2cnc(Nc3ccc(Cl)cc3)nc2n1-c1cccc(OC2CCNCC2)n1] | 452.0 | 1H NMR (400 MHz, cdcl3) δ 8.86 (s, 1H), 7.77 (t, J = 7.9 Hz, 1H), 7.71-7.61 (m, 1H), 7.60-7.51 (m, 2H), 7.37 (d, J = 7.7 Hz, 1H), 7.31 (d, J = 8.7 Hz, 2H), 6.70 (d, J = 8.1 Hz, 1H), 5.09 (tt, J = 8.5, 4.0 Hz, 1H), 3.57 (s, 3H), 3.13 (dt, J = 12.5, 4.6 Hz, 2H), 2.76 (ddd, J = 12.6, 9.5, 3.1 Hz, 2H), 2.01 (dt, J = 13.6, 4.0 Hz, 2H), 1.74-1.62 (m, 4H) |
| 154 | 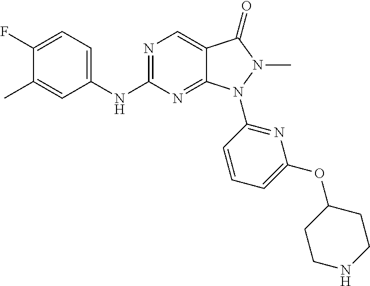 [SMILES: Cc1cc(Nc2ncc3c(=O)n(C)n(-c4cccc(OC5CCNCC5)n4)c3n2)ccc1F] | 450.0 | 1H NMR (400 MHz, cdcl3) δ 8.84 (s, 1H), 7.75 (t, J = 7.9 Hz, 1H), 7.64-7.48 (m, 2H), 7.41 (d, J = 7.7 Hz, 1H), 7.34- 7.28 (m, 1H), 6.98 (t, J = 9.0 Hz, 1H), 6.68 (d, J = 8.1 Hz, 1H), 5.09 (tt, J = 8.6, 4.0 Hz, 1H), 3.57 (s, 3H), 3.13 (dt, J = 12.6, 4.6 Hz, 2H), 2.76 (ddd, J = 12.6, 9.5, 3.1 Hz, 2H), 2.30 (d, J = 2.0 Hz, 3H), 2.02 (dt, J = 13.6, 4.5 Hz, 2H), 1.68 (dtd, J = 13.1, 9.2, 3.9 Hz, 4H) |
| 155 | 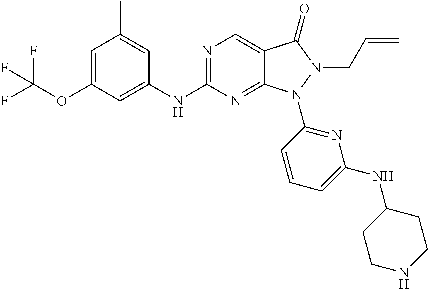 [SMILES: C=CCn1c(=O)c2cnc(Nc3cc(C)cc(OC(F)(F)F)c3)nc2n1-c1cccc(NC2CCNCC2)n1] | 541.0 | 1H NMR (400 MHz, dmso) δ 10.45 (s, 1H), 8.91 (s, 1H), 8.47 (s, 1H), 8.26 (s, 1H), 7.73 (s, 1H), 7.61 (s, 1H), 7.57 (d, J = 7.8 Hz, 1H), 7.07 (d, J = 7.3 Hz, 1H), 6.92 (d, J = 7.4 Hz, 1H), 6.83 (s, 1H), 6.51 (d, J = 8.3 Hz, 1H), 5.71 (dd, J = 16.9, 10.5 Hz, 1H), 5.05 (d, J = 10.4 Hz, 1H), 4.91 (d, J = 17.2 Hz, 1H), 4.60 (d, J = 4.8 Hz, 2H), 3.89 (s, 1H), 3.26 (s, 2H), 3.02 (d, J = 11.2 Hz, 2H), 2.32 (s, 3H), 2.02 (d, J = 12.4 Hz, 2H), 1.58 (d, J = 11.1 Hz, 2H) |
| 156 | 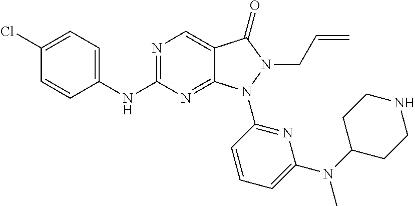 [SMILES: C=CCn1c(=O)c2cnc(Nc3ccc(Cl)cc3)nc2n1-c1cccc(N(C)C2CCNCC2)n1] | 491.0 | 1H NMR (400 MHz, dmso) δ 10.37 (s, 1H), 8.89 (s, 1H), 7.79 (dd, J = 20.1, 8.4 Hz, 3H), 7.37 (d, J = 8.8 Hz, 2H), 6.98 (d, J = 7.5 Hz, 1H), 6.60 (d, J = 8.5 Hz, 1H), 5.70 (td, J = 10.5, 5.1 Hz, 1H), 5.03 (d, J = 10.2 Hz, 1H), 4.89 (d, J = 17.3 Hz, 1H), 4.65 (d, J = 5.0 Hz, 2H), 4.34 (s, 1H), 3.00 (d, J = 11.9 Hz, 2H), 2.85 (s, 3H), 2.54 (d, J = 10.4 Hz, 2H), 1.68- 1.54 (m, 2H), 1.50 (d, J = 10.2 Hz, 2H). |
| 157 | 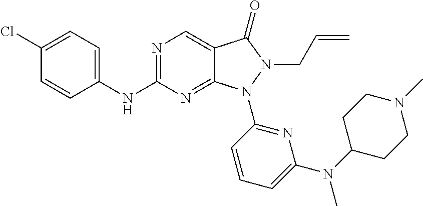 [SMILES: C=CCn1c(=O)c2cnc(Nc3ccc(Cl)cc3)nc2n1-c1cccc(N(C)C2CCN(C)CC2)n1] | 505.0 | 1H NMR (400 MHz, dmso) δ 10.39 (s, 1H), 9.27 (s, 1H), 8.90 (s, 1H), 7.82 (t, J = 9.1 Hz, 3H), 7.38 (d, J = 8.8 Hz, 2H), 7.06 (d, J = 7.6 Hz, 1H), 6.67 (d, J = 8.5 Hz, 1H), 5.70 (dd, J = 16.9, 10.4 Hz, 1H), 5.04 (d, J = 10.1 Hz, 1H), 4.88 (d, J = 17.1 Hz, 1H), 4.65 (s, 3H), 3.50 (d, J = 11.1 Hz, 2H), 3.14 (d, J = 12.0 Hz, 2H), 2.84 (s, 3H), 2.81 (d, J = 4.4 Hz, 3H), 1.96 (d, J = 10.6 Hz, 2H), 1.80 (d, J = 12.1 Hz, 2H). |
| 158 | 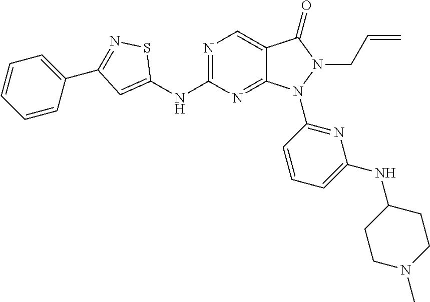 [SMILES: C=CCn1c(=O)c2cnc(Nc3cc(-c4ccccc4)ns3)nc2n1-c1cccc(NC2CCN(C)CC2)n1] | 540.0 | 1H NMR (400 MHz, DMSO-d6) δ 12.39-11.99 (m, 1H), 9.37-9.17 (m, 1H), 9.11-8.94 (m, 1H), 7.99-6.46 (m, 10H), 5.84-5.66 (m, 1H), 5.08 (d, J = 9.8 Hz, 1H), 4.94 (d, J = 16.9 Hz, 1H), 4.68-4.51 (m, 2H), 3.17-3.00 (m, 2H), 2.83-2.69 (m, 3H), 2.18-1.48 (m, 4H). Overlap with solvent and water signal three protons not listed. |
| 159 | 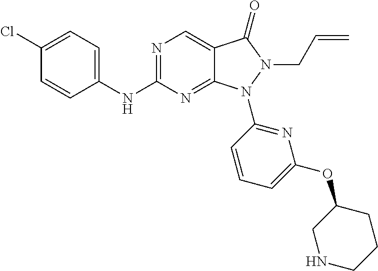 [SMILES: C=CCn1c(=O)c2cnc(Nc3ccc(Cl)cc3)nc2n1-c1cccc(O[C@H]2CCCNC2)n1] | 478.0 | |
| 160 | 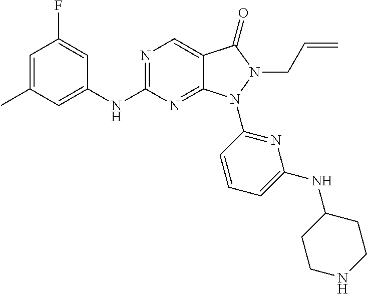 [SMILES: C=CCn1c(=O)c2cnc(Nc3cc(C)cc(F)c3)nc2n1-c1cccc(NC2CCNCC2)n1] | 475.0 | 1H NMR (400 MHz, DMSO-d6) δ 10.35 (s, 1H), 8.89 (s, 1H), 7.59-7.51 (m, 2H), 7.47 (s, 1H), 6.87 (d, J = 7.4 Hz, 1H), 6.82 (d, J = 7.7 Hz, 1H), 6.67 (d, J = 10.4 Hz, 1H), 6.45 (d, J = 8.3 Hz, 1H), 5.79-5.61 (m, 1H), 5.05 (d, J = 10.3 Hz, 1H), 4.91 (d, J = 18.2 Hz, 1H), 4.62 (d, J = 5.2 Hz, 2H), 3.62 (d, J = 15.2 Hz, 1H), 3.33 (s, 2H), 2.91 (d, J = 12.3 Hz, 2H), 2.28 (s, 3H), 1.79 (d, J = 12.4 Hz, 2H), 1.35 (s, 1H), 1.32-1.17 (m, 2H). |
| 161 | 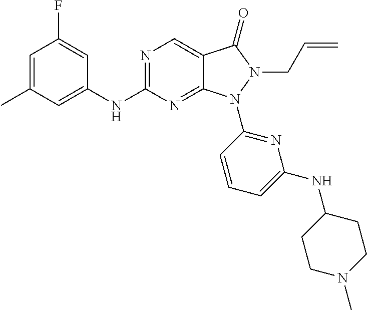 [SMILES: C=CCn1c(=O)c2cnc(Nc3cc(C)cc(F)c3)nc2n1-c1cccc(NC2CCN(C)CC2)n1] | 489.3 | 1H NMR (400 MHz, DMSO-d6) δ 10.37 (s, 1H), 9.47-9.21 (m, 1H), 8.90 (s, 1H), 7.67-7.56 (m, 1H), 7.52 (d, J = 12.2 Hz, 1H), 7.45 (s, 1H), 7.05 (d, J = 7.0 Hz, 1H), 7.01-6.91 (m, 1H), 6.68 (d, J = 9.8 Hz, 1H), 6.59-6.43 (m, 1H), 5.78-5.63 (m, 1H), 5.06 (d, J = 10.3 Hz, 1H), 4.91 (d, J = 17.1 Hz, 1H), 4.68-4.53 (m, 2H), 4.09-3.75 (m, 1H), 3.50-3.27 (m, 2H), 3.22-3.00 (m, 2H), 2.82-2.72 (m, 3H), 2.28 (s, 3H), 2.15-1.88 (m, 2H), 1.70-1.50 (m, 2H). |
| 162 | 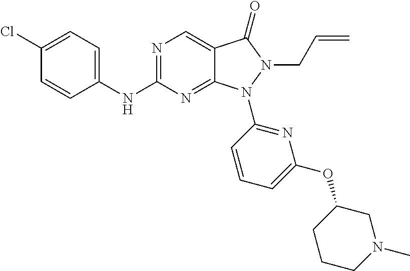 [SMILES: C=CCn1c(=O)c2cnc(Nc3ccc(Cl)cc3)nc2n1-c1cccc(O[C@H]2CCCN(C)C2)n1] | 492.0 | 1H NMR (400 MHz, cd3od) δ 8.85 (s, 1H), 8.01 (t, J = 7.9 Hz, 1H), 7.69 (d, J = 8.9 Hz, 2H), 7.50 (dd, J = 14.3, 7.7 Hz, 1H), 7.29 (d, J = 8.7 Hz, 2H), 6.90 (dd, J = 30.2, 8.1 Hz, 1H), 5.76 (ddt, J = 16.4, 10.9, 5.8 Hz, 1H), 5.52 (s, 1H), 5.11 (d, J = 10.2 Hz, 1H), 5.00 (d, J = 17.1 Hz, 1H), 4.68 (qd, J = 16.2, 5.9 Hz, 2H), 3.72 (d, J = 13.4 Hz, 1H), 3.52 (d, J = 12.6 Hz, 1H), 3.36 (d, J = 21.8 Hz, 1H), 3.04 (q, J = 12.4 Hz, 1H), 2.88 (d, J = 21.7 Hz, 3H), 2.15 (d, J = 13.6 Hz, 2H), 1.83 (q, J = 16.4 Hz, 2H). |
| 163 | 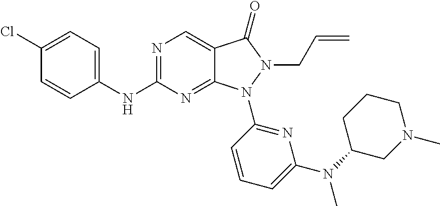 [SMILES: C=CCn1c(=O)c2cnc(Nc3ccc(Cl)cc3)nc2n1-c1cccc(N(C)[C@@H]2CCCN(C)C2)n1] | 505.0 | 1H NMR (400 MHz, dmso) δ 10.40 (s, 1H), 9.63 (s, 1H), 8.90 (s, 1H), 7.88 (t, J = 8.0 Hz, 1H), 7.81 (d, J = 8.6 Hz, 2H), 7.38 (d, J = 8.6 Hz, 2H), 7.10 (d, J = 7.6 Hz, 1H), 6.66 (d, J = 8.5 Hz, 1H), 5.70 (ddt, J = 16.3, 10.7, 5.7 Hz, 1H), 5.04 (d, J = 10.9 Hz, 1H), 4.88 (d, J = 19.1 Hz, 1H), 4.83-4.74 (m, 1H), 4.64 (qd, J = 16.4, 5.9 Hz, 2H), 3.40 (d, J = 11.9 Hz, 1H), 3.30 (d, J = 8.9 Hz, 1H), 3.18 (q, J = 11.1 Hz, 1H), 2.97-2.82 (m, 4H), 2.79 (d, J = 4.5 Hz, 3H), 2.05- 1.90 (m, 1H), 1.80-1.61 (m, 3H). |
| 164 | 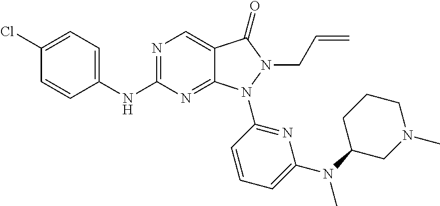 [SMILES: C=CCn1c(=O)c2cnc(Nc3ccc(Cl)cc3)nc2n1-c1cccc(N(C)[C@H]2CCCN(C)C2)n1] | 505.0 | 1H NMR (400 MHz, dmso) δ 10.40 (s, 1H), 9.62 (s, 1H), 8.90 (s, 1H), 7.88 (t, J = 8.0 Hz, 1H), 7.81 (d, J = 8.6 Hz, 2H), 7.38 (d, J = 8.8 Hz, 2H), 7.10 (d, J = 7.6 Hz, 1H), 6.66 (d, J = 8.5 Hz, 1H), 5.70 (ddt, J = 16.2, 10.7, 5.7 Hz, 1H), 5.04 (d, J = 10.3 Hz, 1H), 4.88 (d, J = 17.8 Hz, 1H), 4.84-4.74 (m, 1H), 4.64 (qd, J = 16.4, 5.1 Hz, 2H), 3.40 (d, J = 11.8 Hz, 1H), 3.30 (d, J = 11.1 Hz, 1H), 3.18 (q, J = 11.2 Hz, 1H), 2.94-2.82 (m, 4H), 2.79 (d, J = 4.5 Hz, 3H), 2.05- 1.89 (m, 1H), 1.82-1.62 (m, 3H). |
| 165 | 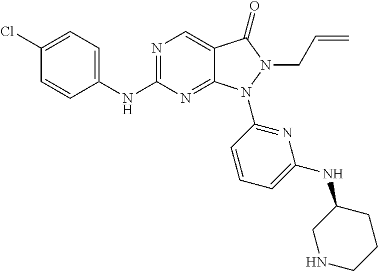 [SMILES: C=CCn1c(=O)c2cnc(Nc3ccc(Cl)cc3)nc2n1-c1cccc(N[C@H]2CCCNC2)n1] | 477.0 | 1H NMR (400 MHz, DMSO-d6) δ 10.38 (s, 1H), 8.89 (s, 1H), 8.73-8.48 (m, 2H), 7.81 (d, J = 8.9 Hz, 2H), 7.71 (t, J = 7.7 Hz, 1H), 7.37 (d, J = 8.9 Hz, 2H), 7.01 (d, J = 7.2 Hz, 1H), 6.96 (d, J = 7.6 Hz, 1H), 6.50 (d, J = 8.2 Hz, 1H), 5.77-5.63 (m, 1H), 5.06 (d, J = 10.2 Hz, 1H), 4.91 (d, J = 17.3 Hz, 1H), 4.61 (d, J = 5.1 Hz, 2H), 4.08-3.93 (m, 1H), 3.34 (d, J = 10.8 Hz, 1H), 3.17 (d, J = 12.7 Hz, 1H), 2.94-2.79 (m, 1H), 2.78- 2.64 (m, 1H), 2.01-1.81 (m, 2H), 1.75-1.61 (m, 1H), 1.57-1.43 (m, 1H). |
| 166 | 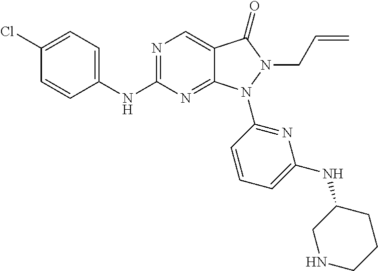 [SMILES: C=CCn1c(=O)c2cnc(Nc3ccc(Cl)cc3)nc2n1-c1cccc(N[C@@H]2CCCNC2)n1] | 477.0 | 1H NMR (400 MHz, DMSO-d6) δ 10.38 (s, 1H), 8.89 (s, 1H), 8.73-8.45 (m, 2H), 7.81 (d, J = 8.9 Hz, 2H), 7.71 (t, J = 7.7 Hz, 1H), 7.37 (d, J = 8.9 Hz, 2H), 7.01 (d, J = 7.2 Hz, 1H), 6.96 (d, J = 7.6 Hz, 1H), 6.50 (d, J = 8.2 Hz, 1H), 5.79-5.62 (m, 1H), 5.06 (d, J = 10.4 Hz, 1H), 4.91 (d, J = 17.2 Hz, 1H), 4.61 (d, J = 4.6 Hz, 2H), 4.11-3.90 (m, 1H), 3.39-3.28 (m, 1H), 3.25-3.11 (m, 1H), 2.93-2.79 (m, 1H), 2.78-2.64 (m, 1H), 2.04-1.81 (m, 2H), 1.76- 1.60 (m, 1H), 1.56-1.43 (m, 1H). |
| 167 | 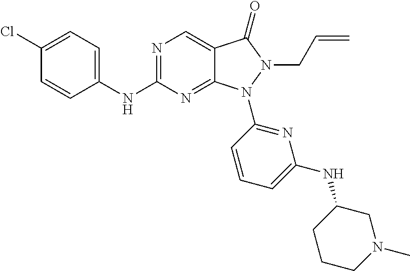 [SMILES: C=CCn1c(=O)c2cnc(Nc3ccc(Cl)cc3)nc2n1-c1cccc(N[C@H]2CCCN(C)C2)n1] | 491.0 | 1H NMR (400 MHz, DMSO-d6) δ 10.38 (s, 1H), 9.89-9.31 (m, 1H), 8.89 (s, 1H), 7.81 (d, J = 8.9 Hz, 2H), 7.72 (t, J = 8.1 Hz, 1H), 7.37 (d, J = 8.9 Hz, 2H), 7.10 (d, J = 7.8 Hz, 1H), 7.04- 6.91 (m, 1H), 6.63-6.43 (m, 1H), 5.84- 5.62 (m, 1H), 5.05 (d, J = 9.6 Hz, 1H), 4.98-4.83 (m, 1H), 4.67-4.52 (m, 2H), 4.21-4.01 (m, 1H), 3.50 (d, J = 11.9 Hz, 1H), 3.44-3.31 (m, 1H), 3.21- 2.55 (m, 5H), 2.11-1.29 (m, 4H). |
| 168 | 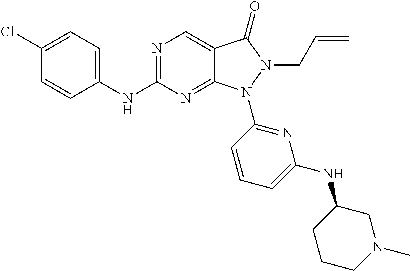 [SMILES: C=CCn1c(=O)c2cnc(Nc3ccc(Cl)cc3)nc2n1-c1cccc(N[C@@H]2CCCN(C)C2)n1] | 491.0 | 1H NMR (400 MHz, Methanol-d4) δ 8.84 (s, 1H), 7.77-7.62 (m, 3H), 7.35- 7.22 (m, 2H), 7.12-6.93 (m, 1H), 6.74- 6.52 (m, 1H), 5.86-5.67 (m, 1H), 5.11 (d, J = 10.3 Hz, 1H), 4.99 (d, J = 17.2 Hz, 1H), 4.79-4.56 (m, 2H), 4.25- 4.08 (m, 1H), 3.83-3.65 (m, 1H), 3.54-3.38 (m, 1H), 3.19-2.58 (m, 5H), 2.32-1.45 (m, 4H). Three exchangeable signals not listed. |
| 169 | 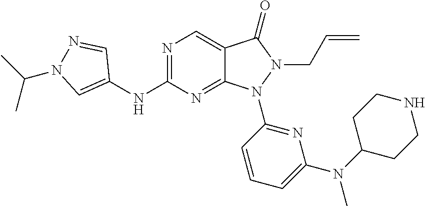 [SMILES: C=CCn1c(=O)c2cnc(Nc3cnn(C(C)C)c3)nc2n1-c1cccc(N(C)C2CCNCC2)n1] | 489.3 | 1H NMR (400 MHz, DMSO-d6) δ 10.28 (s, 1H), 8.78 (s, 1H), 8.61-8.46 (m, 1H), 8.28-8.15 (m, 1H), 7.94 (s, 1H), 7.78 (t, J = 7.9 Hz, 1H), 7.53 (s, 1H), 7.06 (d, J = 7.6 Hz, 1H), 6.72 (d, J = 8.6 Hz, 1H), 5.76-5.60 (m, 1H), 5.03 (d, J = 10.5 Hz, 1H), 4.87 (d, J = 17.4 Hz, 1H), 4.65-4.50 (m, 3H), 4.50- 4.35 (m, 1H), 3.41-3.34 (m, 2H), 3.12- 2.95 (m, 2H), 2.86 (s, 3H), 2.00-1.82 (m, 2H), 1.76 (d, J = 12.0 Hz, 2H), 1.39 (d, J = 6.6 Hz, 6H). |
| 170 | 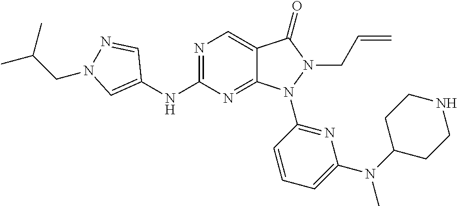 [SMILES: C=CCn1c(=O)c2cnc(Nc3cnn(CC(C)C)c3)nc2n1-c1cccc(N(C)C2CCNCC2)n1] | 503.3 | 1H NMR (400 MHz, DMSO-d6) δ 10.29 (s, 1H), 8.78 (s, 1H), 8.56 (d, J = 10.9 Hz, 1H), 8.23 (d, J = 9.8 Hz, 1H), 7.90 (s, 1H), 7.79 (t, J = 8.1 Hz, 1H), 7.55 (s, 1H), 7.03 (d, J = 7.4 Hz, 1H), 6.72 (d, J = 8.6 Hz, 1H), 5.76-5.63 (m, 1H), 5.03 (d, J = 9.8 Hz, 1H), 4.88 (d, J = 17.3 Hz, 1H), 4.60 (d, J = 5.6 Hz, 3H), 3.86 (d, J = 7.2 Hz, 2H), 3.41-3.33 (m, 2H), 3.03 (q, J = 11.3 Hz, 2H), 2.86 (s, 3H), 2.10-1.98 (m, 1H), 1.98-1.82 (m, 2H), 1.76 (d, J = 12.9 Hz, 2H), 0.83 (d, J = 6.7 Hz, 6H). |
| 171 | 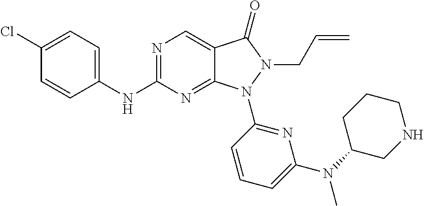 [SMILES: C=CCn1c(=O)c2cnc(Nc3ccc(Cl)cc3)nc2n1-c1cccc(N(C)[C@@H]2CCCNC2)n1] | 491.0 | 1H NMR (500 MHz, DMSO) δ 10.39 (s, 1H), 8.91 (s, 1H), 8.78 (d, J = 9.3 Hz, 1H), 8.61 (q, J = 9.6 Hz, 1H), 7.87 (t, J = 8.0 Hz, 1H), 7.82 (d, J = 8.9 Hz, 2H), 7.39 (d, J = 8.9 Hz, 2H), 7.09 (d, J = 7.6 Hz, 1H), 6.65 (d, J = 8.5 Hz, 1H), 5.71 (ddt, J = 16.3, 10.3, 5.7 Hz, 1H), 5.04 (dd, J = 10.4, 1.5 Hz, 1H), 4.89 (dt, J = 17.2, 1.6 Hz, 1H), 4.80-4.72 (m, 1H), 4.66 (qd, J = 15.9, 5.7 Hz, 2H), 3.24 (d, J = 12.7 Hz, 1H), 3.19-3.06 (m, 2H), 2.90 (s, 3H), 2.87-2.77 (m, 1H), 1.97- 1.89 (m, 1H), 1.89-1.79 (m, 1H), 1.77- |
| | | | 1.66 (m, 2H). |
| 172 | 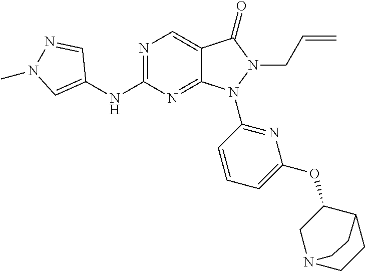 [SMILES: C=CCn1c(=O)c2cnc(Nc3cnn(C)c3)nc2n1-c1cccc(O[C@H]2CN3CCC2CC3)n1] | 474.0 | 1H NMR (400 MHz, dmso) δ 10.21 (d, J = 123.3 Hz, 1H), 9.49 (s, 1H), 8.84 (d, J = 30.2 Hz, 1H), 8.16-7.95 (m, 1H), 7.87 (d, J = 30.0 Hz, 1H), 7.58-7.47 (m, 2H), 6.91 (d, J = 8.1 Hz, 1H), 5.78- 5.62 (m, 1H), 5.14 (s, 1H), 5.07 (d, J = 10.2 Hz, 1H), 4.95 (d, J = 17.2 Hz, 1H), 4.56 (dd, J = 18.2, 5.7 Hz, 2H), 3.81 (s, 3H), 3.78-3.70 (m, 1H), 3.41 (d, J = 13.3 Hz, 1H), 3.32-3.20 (m, 4H), 2.37 (s, 1H), 2.13 (s, 1H), 1.91 (s, 2H), 1.80 (s, 1H) |
| 173 | 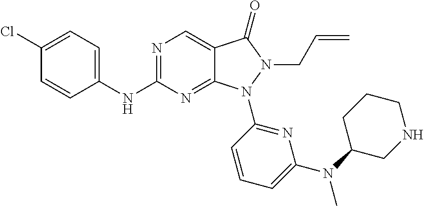 [SMILES: C=CCn1c(=O)c2cnc(Nc3ccc(Cl)cc3)nc2n1-c1cccc(N(C)[C@H]2CCCNC2)n1] | 491.0 | 1H NMR (400 MHz, dmso) δ 10.40 (s, 1H), 9.62 (s, 1H), 8.90 (s, 1H), 7.88 (t, J = 8.0 Hz, 1H), 7.81 (d, J = 8.6 Hz, 2H), 7.38 (d, J = 8.8 Hz, 2H), 7.10 (d, J = 7.6 Hz, 1H), 6.66 (d, J = 8.5 Hz, 1H), 5.70 (ddt, J = 16.2, 10.7, 5.7 Hz, 1H), 5.04 (d, J = 10.3 Hz, 1H), 4.88 (d, J = 17.8 Hz, 1H), 4.84-4.74 (m, 1H), 4.64 (qd, J = 16.4, 5.1 Hz, 2H), 3.40 (d, J = 11.8 Hz, 1H), 3.30 (d, J = 11.1 Hz, 1H), 3.18 (q, J = 11.2 Hz, 1H), 2.94-2.82 (m, 4H), 2.79 (d, J = 4.5 Hz, 3H), 2.05- 1.89 (m, 1H), 1.82-1.62 (m, 3H). |
| 174 | 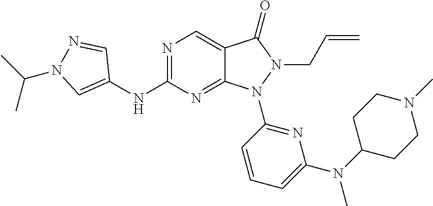 [SMILES: C=CCn1c(=O)c2cnc(Nc3cnn(C(C)C)c3)nc2n1-c1cccc(N(C)C2CCN(C)CC2)n1] | 503.3 | 1H NMR (400 MHz, DMSO-d6) δ 10.28 (s, 1H), 9.26 (s, 1H), 8.78 (s, 1H), 7.94 (s, 1H), 7.79 (t, J = 7.9 Hz, 1H), 7.53 (s, 1H), 7.07 (d, J = 7.2 Hz, 1H), 6.69 (d, J = 8.6 Hz, 1H), 5.77-5.61 (m, 1H), 5.03 (d, J = 10.5 Hz, 1H), 4.87 (d, J = 17.7 Hz, 1H), 4.68-4.55 (m, 3H), 4.51-4.37 (m, 1H), 3.53-3.46 (m, 2H), 3.21-3.07 (m, 2H), 2.84 (s, 3H), 2.81 (d, J = 4.7 Hz, 3H), 2.03-1.88 (m, 2H), 1.80 (d, J = 11.2 Hz, 2H), 1.39 (d, J = 6.6 Hz, 6H). |
| 175 | 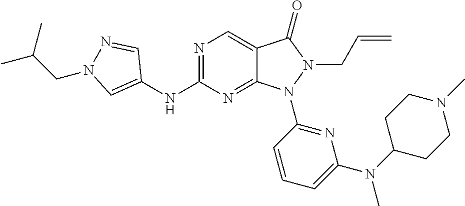 [SMILES: C=CCn1c(=O)c2cnc(Nc3cnn(CC(C)C)c3)nc2n1-c1cccc(N(C)C2CCN(C)CC2)n1] | 517.3 | 1H NMR (400 MHz, DMSO-d6) δ 10.30 (s, 1H), 9.32 (s, 1H), 8.78 (s, 1H), 7.89 (s, 1H), 7.80 (t, J = 8.0 Hz, 1H), 7.56 (s, 1H), 7.05 (d, J = 7.5 Hz, 1H), 6.69 (d, J = 8.7 Hz, 1H), 5.76-5.63 (m, 1H), 5.03 (d, J = 9.9 Hz, 1H), 4.87 (d, J = 17.3 Hz, 1H), 4.69-4.55 (m, 3H), 3.86 (d, J = 7.1 Hz, 2H), 3.53-3.50 (m, 2H), 3.21-3.07 (m, 2H), 2.84 (s, 3H), 2.81 (d, J = 4.5 Hz, 3H), 2.11-1.88 (m, 3H), 1.80 (d, J = 11.9 Hz, 2H), 0.83 (d, J = 6.7 Hz, 6H). |
| 176 | 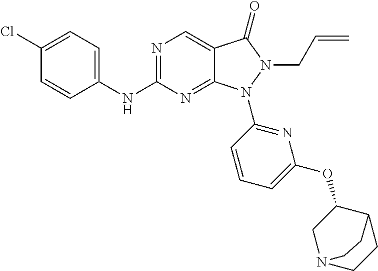 [SMILES: C=CCn1c(=O)c2cnc(Nc3ccc(Cl)cc3)nc2n1-c1cccc(O[C@H]2CN3CCC2CC3)n1] | 504.0 | 1H NMR (400 MHz, dmso) δ 10.46 (s, 1H), 9.64 (s, 1H), 8.93 (s, 1H), 8.09 (s, 1H), 7.79 (d, J = 8.8 Hz, 2H), 7.49 (d, J = 7.7 Hz, 1H), 7.39 (d, J = 8.8 Hz, 2H), 6.90 (d, J = 8.2 Hz, 1H), 5.77-5.63 (m, 1H), 5.13 (s, 1H), 5.07 (d, J = 10.2 Hz, 1H), 4.96 (d, J = 17.0 Hz, 1H), 4.60 (d, J = 14.9 Hz, 2H), 3.73 (s, 1H), 3.38 (d, J = 13.8 Hz, 1H), 3.26 (d, J = 7.9 Hz, 4H), 2.38 (s, 1H), 2.14 (s, 1H), 1.91 (s, 2H), 1.79 (s, 1H). |
| 177 | 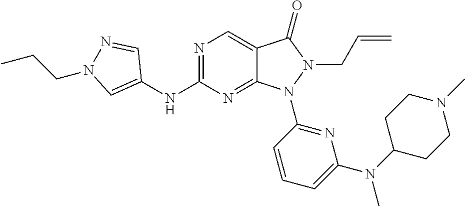 [SMILES: C=CCn1c(=O)c2cnc(Nc3cnn(CCC)c3)nc2n1-c1cccc(N(C)C2CCN(C)CC2)n1] | 503.3 | 1H NMR (400 MHz, dmso) δ 10.28 (s, 1H), 8.77 (s, 1H), 7.92 (s, 1H), 7.76 (t, J = 8.0 Hz, 1H), 7.01 (d, J = 7.5 Hz, 1H), 6.63 (d, J = 8.6 Hz, 1H), 5.76 (s, 3H), 5.10-4.80 (m, 2H), 4.60 (d, J = 5.4 Hz, 2H), 4.01 (t, J = 6.9 Hz, 2H), 2.85 (s, 4H), 2.54 (s, 5H), 2.33 (s, 1H), 1.91- 1.54 (m, 6H), 0.82 (t, J = 7.4 Hz, 3H). |
| 178 | 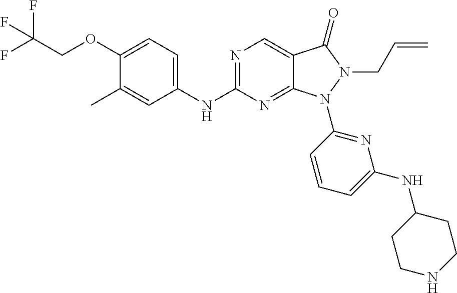 [SMILES: C=CCn1c(=O)c2cnc(Nc3ccc(OCC(F)(F)F)c(C)c3)nc2n1-c1cccc(NC2CCNCC2)n1] | 555.3 | 1H NMR (400 MHz, DMSO-d6) δ 10.16 (s, 1H), 8.82 (s, 1H), 8.57-8.43 (m, 1H), 8.36-8.22 (m, 1H), 7.74 (s, 1H), 7.63 (t, J = 7.9 Hz, 1H), 7.48-7.39 (m, 1H), 7.03 (s, 1H), 7.00 (d, J = 8.9 Hz, 1H), 6.95 (d, J = 7.5 Hz, 1H), 6.48 (d, J = 8.3 Hz, 1H), 5.78-5.61 (m, 1H), 5.05 (d, J = 10.3 Hz, 1H), 4.91 (d, J = 17.1 Hz, 1H), 4.70 (q, J = 8.9 Hz, 2H), 4.61 (s, 2H), 3.97-3.84 (m, 1H), 3.35- 3.19 (m, 2H), 3.13-2.95 (m, 2H), 2.17 (s, 3H), 2.08-1.96 (m, 2H), 1.69-1.48 (m, 2H). |
| 179 | 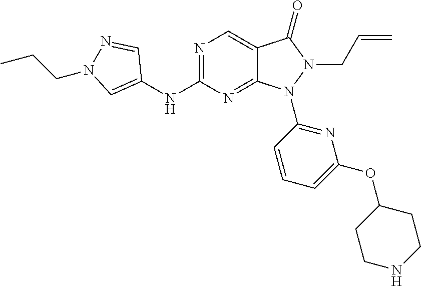 [SMILES: C=CCn1c(=O)c2cnc(Nc3cnn(CCC)c3)nc2n1-c1cccc(OC2CCNCC2)n1] | 476.3 | 1H NMR (400 MHz, dmso) δ 10.35 (s, 1H), 8.58-8.38 (m, 2H), 8.01 (t, J = 7.9 Hz, 1H), 7.87 (s, 1H), 7.54 (s, 1H), 7.47 (d, J = 7.7 Hz, 1H), 6.87 (d, J = 8.2 Hz, 1H), 5.70 (td, J = 10.6, 5.4 Hz, 1H), 5.19 (s, 1H), 5.05 (d, J = 10.0 Hz, 1H), 4.92 (d, J = 16.8 Hz, 1H), 4.56 (d, J = 5.5 Hz, 2H), 4.02 (t, J = 6.8 Hz, 2H), 3.23 (s, 2H), 2.19-1.66 (m, 6H), 0.80 (t, J = 7.3 Hz, 3H). |
| 180 | 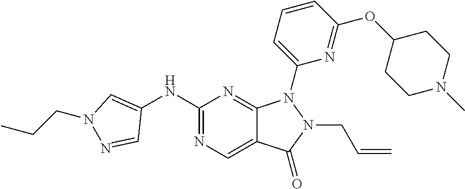 [SMILES: C=CCn1c(=O)c2cnc(Nc3cnn(CCC)c3)nc2n1-c1cccc(OC2CCN(C)CC2)n1] | 490.0 | 1H NMR (400 MHz, dmso) δ 10.35 (s, 1H), 8.80 (s, 1H), 8.08-7.91 (m, 1H), 7.88 (d, J = 4.7 Hz, 1H), 7.64-7.40 (m, 2H), 6.86 (t, J = 7.8 Hz, 1H), 5.70 (tt, J = 10.6, 5.5 Hz, 1H), 5.06 (dd, J = 10.4, 4.0 Hz, 1H), 4.92 (d, J = 17.2 Hz, 1H), 4.56 (s, 2H), 4.02 (t, J = 6.8 Hz, 2H), 3.17 (d, J = 9.9 Hz, 2H), 2.81 (dd, J = 13.5, 4.4 Hz, 3H), 2.17-1.71 (m, 6H), 0.81 (t, J = 7.1 Hz, 3H). |
| 181 | 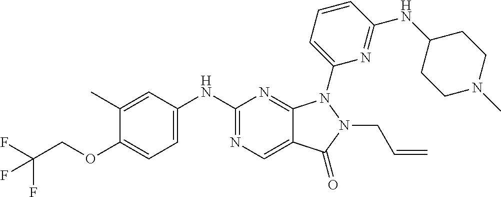 [SMILES: C=CCn1c(=O)c2cnc(Nc3ccc(OCC(F)(F)F)c(C)c3)nc2n1-c1cccc(NC2CCN(C)CC2)n1] | 569.0 | 1H NMR (400 MHz, DMSO-d6) δ 10.15 (s, 1H), 9.50-9.23 (m, 1H), 8.82 (s, 1H), 7.79-7.53 (m, 2H), 7.43 (d, J = 8.5 Hz, 1H), 7.07-6.90 (m, 3H), 6.57- 6.39 (m, 1H), 5.78-5.60 (m, 1H), 5.05 (d, J = 10.4 Hz, 1H), 4.91 (d, J = 16.9 Hz, 1H), 4.70 (q, J = 8.7 Hz, 2H), 4.61 (s, 2H), 4.12-3.70 (m, 1H), 3.53-3.00 (m, 4H), 2.82-2.68 (m, 3H), 2.17 (s, 3H), 2.12-1.49 (m, 4H). |
| 182 | 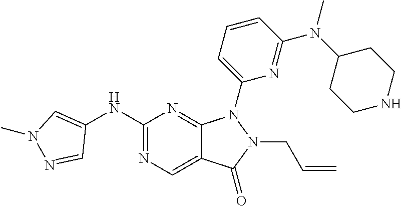 [SMILES: C=CCn1c(=O)c2cnc(Nc3cnn(C)c3)nc2n1-c1cccc(N(C)C2CCNCC2)n1] | 461.0 | 1H NMR (400 MHz, dmso) δ 10.29 (s, 1H), 8.78 (s, 1H), 8.48 (d, J = 4.5 Hz, 1H), 8.22-8.07 (m, 1H), 7.93-7.81 (m, 2H), 7.56 (s, 1H), 7.04 (d, J = 7.5 Hz, 1H), 6.71 (d, J = 8.5 Hz, 1H), 5.75- 5.62 (m, 1H), 5.03 (d, J = 10.1 Hz, 1H), 4.88 (d, J = 16.8 Hz, 1H), 4.64-4.52 (m, 3H), 3.80 (s, 3H), 3.04 (q, J = 11.4 Hz, 2H), 2.87 (s, 3H), 2.57-2.52 (m, 2H), 1.99-1.83 (m, 2H), 1.81-1.70 (m, 2H). |
| 183 | 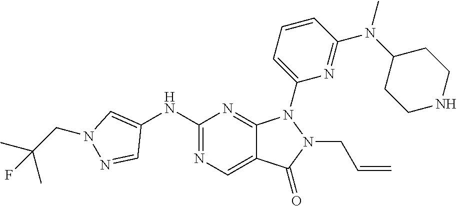 [SMILES: C=CCn1c(=O)c2cnc(Nc3cnn(CC(C)(C)F)c3)nc2n1-c1cccc(N(C)C2CCNCC2)n1] | 521.0 | 1H NMR (500 MHz, DMSO) δ 10.32 (s, 1H), 8.78 (s, 1H), 8.49 (d, J = 9.3 Hz, 1H), 8.15 (q, J = 9.7 Hz, 1H), 7.97 (s, 1H), 7.77 (t, J = 8.0 Hz, 1H), 7.58 (s, 1H), 7.03 (d, J = 7.6 Hz, 1H), 6.70 (d, J = 8.5 Hz, 1H), 5.73-5.64 (m, 1H), 5.03 (d, J = 10.3 Hz, 1H), 4.87 (d, J = 17.1 Hz, 1H), 4.65-4.50 (m, 3H), 4.28 (d, J = 21.5 Hz, 2H), 3.04 (q, J = 11.8 Hz, 2H), 2.86 (s, 3H), 2.54-2.52 (m, 2H), 1.97-1.84 (m, 2H), 1.76 (d, J = 12.1 Hz, 2H), 1.27 (d, J = 21.4 Hz, 6H). |
| 184 | 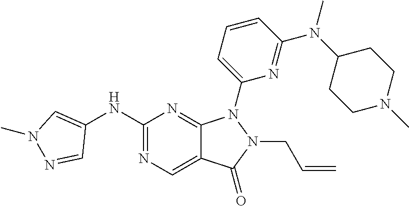 | 475.3 | 1H NMR (400 MHz, dmso) δ 10.29 (s, 1H), 9.46 (s, 1H), 8.78 (s, 1H), 7.91- 7.81 (m, 2H), 7.56 (s, 1H), 7.05 (d, J = 7.6 Hz, 1H), 6.68 (d, J = 8.4 Hz, 1H), 5.69 (ddt, J = 16.3, 10.6, 5.3 Hz, 1H), 5.03 (d, J = 10.3 Hz, 1H), 4.87 (d, J = 17.2 Hz, 1H), 4.71-4.54 (m, 3H), 3.80 (s, 3H), 3.50 (d, J = 11.1 Hz, 2H), 3.14 (q, J = 10.1 Hz, 2H), 2.85 (s, 3H), 2.81 (d, J = 4.5 Hz, 3H), 1.98 (q, J = 11.8 Hz, 2H), 1.80 (d, J = 12.4 Hz, 2H). |
| 185 | 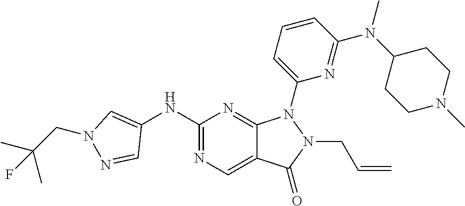 [SMILES: C=CCn1c(=O)c2cnc(Nc3cnn(CC(C)(C)F)c3)nc2n1-c1cccc(N(C)C2CCN(C)CC2)n1] | 535.3 | 1H NMR (500 MHz, DMSO) δ 10.33 (s, 1H), 9.58 (s, 1H), 8.79 (s, 1H), 7.97 (s, 1H), 7.78 (t, J = 8.0 Hz, 1H), 7.58 (s, 1H), 7.03 (d, J = 7.6 Hz, 1H), 6.67 (d, J = 8.5 Hz, 1H), 5.74-5.64 (m, 1H), 5.03 (d, J = 10.3 Hz, 1H), 4.87 (d, J = 17.2 Hz, 1H), 4.62 (d, J = 5.7 Hz, 3H), 4.28 (d, J = 21.5 Hz, 2H), 3.50 (d, J = 11.6 Hz, 2H), 3.14 (q, J = 10.5 Hz, 2H), 2.85 (s, 3H), 2.81 (d, J = 4.3 Hz, 3H), 2.05- 1.92 (m, 2H), 1.80 (d, J = 12.8 Hz, 2H), 1.26 (d, J = 21.4 Hz, 6H). |
| 186 | 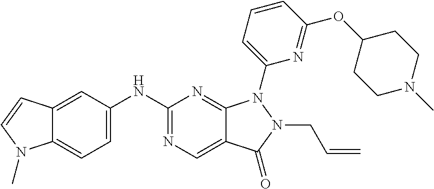 [SMILES: C=CCn1c(=O)c2cnc(Nc3ccc4c(ccn4C)c3)nc2n1-c1cccc(OC2CCN(C)CC2)n1] | 511.0 | 1H NMR (400 MHz, dmso) δ 10.27 (s, 1H), 9.39 (d, J = 22.0 Hz, 1H), 8.84 (s, 1H), 7.98 (dt, J = 10.1, 8.0 Hz, 2H), 7.55 (d, J = 7.7 Hz, 1H), 7.38 (s, 2H), 7.32 (s, 1H), 6.82 (t, J = 8.8 Hz, 1H), 6.38 (t, J = 2.6 Hz, 1H), 5.70 (ddq, J = 16.6, 11.8, 5.8 Hz, 1H), 5.29-5.02 (m, 2H), 4.94 (d, J = 17.1 Hz, 1H), 4.61 (s, 2H), 3.78 (s, 3H), 3.47 (s, 1H), 3.33 (d, J = 10.7 Hz, 1H), 3.22-3.08 (m, 2H), 2.80 (dd, J = 9.8, 4.7 Hz, 3H), 2.27 (d, J = 12.8 Hz, 1H), 2.12 (d, J = 15.1 Hz, 1H), 2.01 |
| | | | (t, J = 14.0 Hz, 1H), 1.78 (q, J = 10.2 |
| | | | Hz, 1H) |
| 187 | 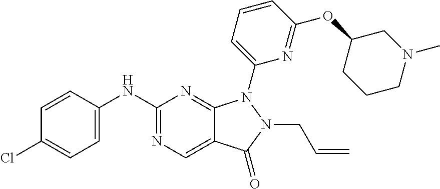 [SMILES: C=CCn1c(=O)c2cnc(Nc3ccc(Cl)cc3)nc2n1-c1cccc(O[C@@H]2CCCN(C)C2)n1] | 492.0 | 1H NMR (400 MHz, dmso) δ 8.92 (s, 1H), 8.08 (s, 1H), 7.79 (d, J = 8.7 Hz, 3H), 7.53-7.45 (m, 1H), 7.40 (d, J = 8.6 Hz, 3H), 6.85 (d, J = 8.2 Hz, 1H), 5.71 (dd, J = 17.1, 10.6 Hz, 1H), 5.33 (d, J = 56.3 Hz, 1H), 5.06 (d, J = 10.2 Hz, 1H), 4.94 (d, J = 17.4 Hz, 1H), 4.60 (d, J = 8.5 Hz, 3H), 3.62 (d, J = 12.4 Hz, 1H), 3.16-2.87 (m, 2H), 2.82 (d, J = 4.5 Hz, 1H), 2.76 (d, J = 4.6 Hz, 2H), 2.06- 1.63 (m, 5H). |
| 188 | 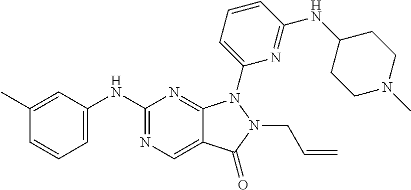 | 471.0 | 1H NMR (400 MHz, DMSO-d6) δ 10.20 (s, 1H), 9.47-9.19 (m, 1H), 8.85 (s, 1H), 7.76 (s, 1H), 7.69-7.58 (m, 1H), 7.44 (d, J = 8.0 Hz, 1H), 7.17 (t, J = 7.8 Hz, 1H), 7.08-6.99 (m, 1H), 6.97 (d, J = 7.5 Hz, 1H), 6.84 (d, J = 7.6 Hz, 1H), 6.58-6.35 (m, 1H), 5.70 (ddt, J = 16.0, 10.6, 5.8 Hz, 1H), 5.06 (d, J = 10.4 Hz, 1H), 4.91 (d, J = 17.0 Hz, 1H), 4.67-4.54 (m, 2H), 4.10-3.77 (m, 1H), 3.54-3.24 (m, 2H), 3.21-2.99 (m, 2H), 2.81-2.74 (m, 3H), 2.28 (s, 3H), |
| | | | 2.17-1.52 (m, 4H). |
| 189 | 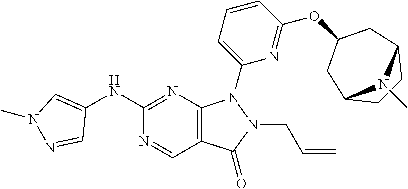 | 488.0 | 1H NMR (500 MHz, DMSO-d6) δ 10.39-9.97 (m, 1H), 8.94-8.73 (m, 1H), 8.04-7.90 (m, 1H), 7.88-7.79 (m, 1H), 7.56-7.49 (m, 1H), 7.41 (d, J = 7.6 Hz, 1H), 6.81-6.70 (m, 1H), 5.71 (ddt, J = 16.2, 10.5, 5.8 Hz, 1H), 5.17 (dq, J = 10.7, 5.6, 5.0 Hz, 1H), 5.06 (d, J = 10.2 Hz, 1H), 5.00-4.87 (m, 1H), 4.69-4.44 (m, 2H), 3.80 (s, 3H), 3.13 (s, 2H), 2.20 (s, 3H), 2.02-1.86 (m, 4H), 1.66 (t, J = 11.1 Hz, 2H), 1.60- 1.46 (m, 2H) |
| 190 | 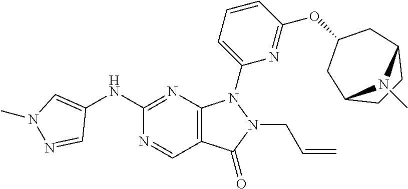 | 488.0 | 1H NMR (500 MHz, DMSO-d6) δ 10.31 (s, 1H), 8.78 (s, 1H), 8.00 (t, J = 7.7 Hz, 1H), 7.87 (d, J = 33.7 Hz, 1H), 7.51 (d, J = 11.9 Hz, 1H), 7.42 (d, J = 7.3 Hz, 1H), 6.77 (d, J = 8.0 Hz, 1H), 5.75-5.61 (m, 1H), 5.13 (s, 1H), 5.03 (d, J = 10.1 Hz, 1H), 4.89 (d, J = 17.1 Hz, 1H), 4.55 (s, 2H), 3.80 (s, 3H), 3.01 (s, 2H), 2.17 (s, 3H), 2.05 (d, J = 14.2 Hz, 2H), 1.94 (s, 4H), 1.70 (d, J = 14.3 Hz, 2H). |
| 191 | 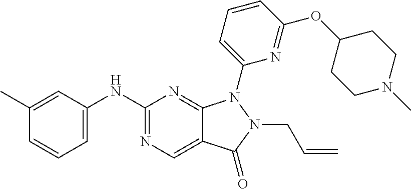 | 472.0 | 1H NMR (400 MHz, dmso) δ 10.26 (s, 1H), 8.88 (s, 1H), 7.92 (t, J = 7.8 Hz, 1H), 7.70 (s, 1H), 7.45 (d, J = 7.1 Hz, 3H), 7.19 (t, J = 7.7 Hz, 1H), 6.82 (dd, J = 26.1, 7.6 Hz, 3H), 5.71 (tt, J = 10.3, 5.3 Hz, 1H), 5.05 (d, J = 10.2 Hz, 1H), 4.92 (d, J = 16.8 Hz, 3H), 4.61 (d, J = 4.3 Hz, 3H), 2.59 (s, 3H), 2.29 (s, 4H), 2.16 (s, 6H), 1.92 (s, 3H), 1.67 (d, J = 9.1 Hz, 3H). |
| 192 | 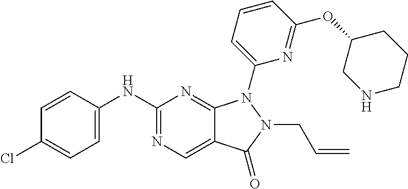 [SMILES: C=CCn1c(=O)c2cnc(Nc3ccc(Cl)cc3)nc2n1-c1cccc(O[C@@H]2CCCNC2)n1] | 478.0 | H NMR (400 MHz, dmso) δ 10.45 (s, 1H), 8.92 (s, 1H), 8.84 (s, 1H), 8.64 (s, 1H), 8.07 (t, J = 7.9 Hz, 1H), 7.79 (d, J = 8.9 Hz, 2H), 7.49 (d, J = 7.7 Hz, 1H), 7.39 (d, J = 8.9 Hz, 2H), 6.85 (d, J = 8.2 Hz, 1H), 5.71 (ddt, J = 16.2, 10.5, 5.8 Hz, 1H), 5.26 (s, 1H), 5.06 (d, J = 10.3 Hz, 1H), 4.94 (d, J = 17.2 Hz, 1H), 4.59 (dq, J = 16.3, 8.4 Hz, 2H), 3.31 (d, J = 30.2 Hz, 3H), 3.09 (s, 2H), 1.98-1.61 (m, 4H). |
| 193 | 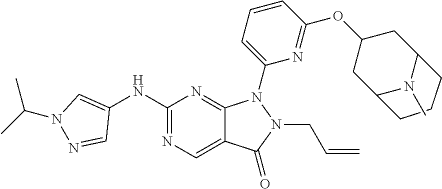 [SMILES: C=CCn1c(=O)c2cnc(Nc3cnn(C(C)C)c3)nc2n1-c1cccc(OC2CC3CCCC(C2)N3C)n1] | 530.3 | 1H NMR (400 MHz, dmso) δ 10.34 (s, 1H), 9.50 (s, 1H), 8.80 (s, 1H), 8.05- 7.85 (m, 2H), 7.51 (d, J = 10.2 Hz, 2H), 6.86 (dd, J = 8.1, 4.1 Hz, 1H), 5.70 (dd, J = 16.9, 10.3 Hz, 1H), 5.27 (t, J = 6.9 Hz, 1H), 5.06 (d, J = 10.2 Hz, 1H), 4.93 (d, J = 17.0 Hz, 1H), 4.62-4.40 (m, 4H), 3.52 (s, 3H), 2.87 (dd, J = 21.2, 4.9 Hz, 4H), 2.71-2.52 (m, 3H), 2.18- 1.75 (m, 7H), 1.39 (d, J = 6.6 Hz, 8H). |
| 194 | 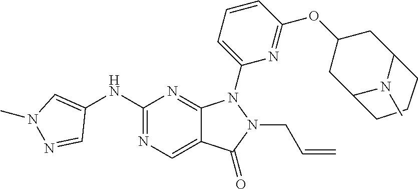 [SMILES: C=CCn1c(=O)c2cnc(Nc3cnn(C)c3)nc2n1-c1cccc(OC2CC3CCCC(C2)N3C)n1] | 502.0 | 1H NMR (400 MHz, dmso) δ 10.33 (s, 1H), 8.79 (s, 1H), 8.07-7.75 (m, 2H), 7.51 (d, J = 12.9 Hz, 1H), 7.40 (d, J = 7.6 Hz, 1H), 6.79 (d, J = 8.1 Hz, 1H), 5.70 (d, J = 6.5 Hz, 1H), 5.34-5.23 (m, 1H), 5.04 (d, J = 10.7 Hz, 1H), 4.90 (d, J = 16.8 Hz, 1H), 4.57 (d, J = 5.7 Hz, 2H), 3.80 (s, 3H), 2.90 (s, 2H), 2.48-2.09 (m, 7H), 1.87 (t, J = 13.3 Hz, 2H), 1.45 (d, J = 8.5 Hz, 3H), 1.12 (d, J = 9.3 Hz, 2H). |
| 195 | 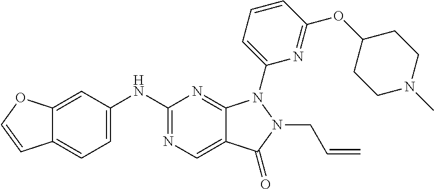 [SMILES: C=CCn1c(=O)c2cnc(Nc3ccc4ccoc4c3)nc2n1-c1cccc(OC2CCN(C)CC2)n1] | 498.0 | 1H NMR (500 MHz, DMSO) δ 10.49 (s, 1H), 9.41 (d, J = 28.6 Hz, 1H), 8.93 (s, 1H), 8.25 (s, 1H), 8.00 (dt, J = 11.9, 7.9 Hz, 1H), 7.94 (d, J = 2.2 Hz, 1H), 7.58 (d, J = 8.4 Hz, 1H), 7.53 (d, J = 7.7 Hz, 1H), 7.49 (dd, J = 8.4, 1.6 Hz, 1H), 6.91 (d, J = 2.4 Hz, 1H), 6.88 (dd, J = 9.9, 8.1 Hz, 1H), 5.72 (ddt, J = 16.7, 12.3, 5.3 Hz, 1H), 5.32-5.10 (m, 1H), 5.08 (ddd, J = 10.4, 6.3, 1.5 Hz, 1H), 4.96 (dt, J = 17.1, 1.5 Hz, 1H), 4.61 (t, J = 6.8 Hz, 2H), 3.49 (d, J = 12.4 Hz, 1H), 3.39- |
| | | | 3.30 (m, 1H), 3.17 (td, J = 12.8, 9.3 Hz, |
| | | | 2H), 2.81 (dd, J = 15.8, 4.7 Hz, 3H), |
| | | | 2.29 (d, J = 13.4 Hz, 1H), 2.14 (d, J = |
| | | | 14.9 Hz, 1H), 2.02 (t, J = 14.0 Hz, 1H), |
| | | | 1.80 (qd, J = 14.3, 3.6 Hz, 1H). |
| 196 | 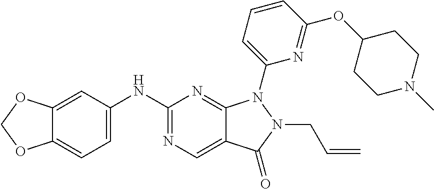 [SMILES: C=CCn1c(=O)c2cnc(Nc3ccc4c(c3)OCO4)nc2n1-c1cccc(OC2CCN(C)CC2)n1] | 501.3 | H NMR (400 MHz, dmso) δ 10.15 (s, 1H), 8.82 (s, 1H), 7.53 (t, J = 7.9 Hz, 1H), 7.49 (s, 1H), 7.13 (d, J = 8.5 Hz, 1H), 6.86 (dd, J = 8.0, 3.6 Hz, 2H), 6.78 (d, J = 7.6 Hz, 1H), 6.43 (d, J = 8.3 Hz, 1H), 5.99 (s, 2H), 5.70 (ddt, J = 16.2, 10.7, 5.7 Hz, 1H), 5.08-4.99 (m, 1H), 4.99-4.86 (m, 1H), 4.60 (s, 2H), 3.57 (s, 1H), 2.70 (d, J = 11.8 Hz, 2H), 2.15 (s, 3H), 1.96 (t, J = 11.3 Hz, 2H), 1.82 (d, J = 9.7 Hz, 2H), 1.43 (qd, J = 11.4, 3.5 Hz, 2H). |
| 197 | 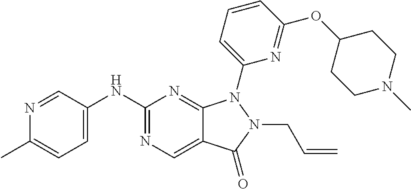 | 473.0 | 1H NMR (400 MHz, dmso) δ 10.62 (s, 1H), 9.56-9.34 (m, 1H), 9.01-8.87 (m, 2H), 8.22 (s, 1H), 8.01 (q, J = 8.2 Hz, 1H), 7.58-7.40 (m, 2H), 6.93- 6.79 (m, 1H), 5.79-5.63 (m, 1H), 5.26 (s, 1H), 5.15-5.02 (m, 2H), 4.93 (d, J = 17.0 Hz, 1H), 4.66-4.54 (m, 2H), 3.47 (d, 1H), 3.33 (d, 1H), 3.25-3.07 (m, 2H), 2.87-2.75 (m, 3H), 2.53 (s, 3H), 2.26 (d, J = 11.9 Hz, 1H), 2.16-1.94 (m, 2H), 1.86-1.71 (m, 1H). |
| 198 | 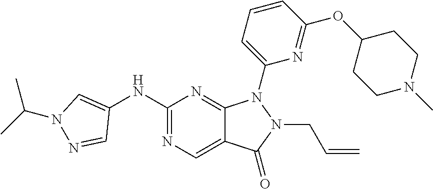 [SMILES: C=CCn1c(=O)c2cnc(Nc3cnn(C(C)C)c3)nc2n1-c1cccc(OC2CCN(C)CC2)n1] | 490.0 | 1H NMR (400 MHz, dmso) δ 10.32 (s, 1H), 8.79 (s, 1H), 7.98-7.87 (m, 2H), 7.51 (s, 1H), 7.43 (d, J = 7.6 Hz, 1H), 6.82 (d, J = 8.1 Hz, 1H), 5.79-5.63 (m, 1H), 5.05 (d, J = 10.2 Hz, 1H), 4.91 (d, J = 16.4 Hz, 2H), 4.55 (d, J = 5.6 Hz, 2H), 4.43 (dt, J = 13.0, 6.6 Hz, 1H), 2.60 (s, 2H), 2.16 (s, 5H), 1.92 (s, 2H), 1.75- 1.58 (m, 2H), 1.39 (d, J = 6.7 Hz, 6H). |
| 199 | 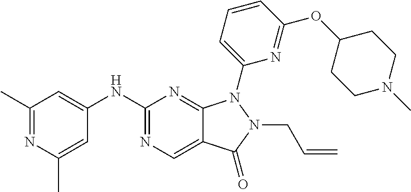 | 487.3 | 1H NMR (500 MHz, DMSO) δ 11.54 (s, 1H), 9.97 (d, J = 55.7 Hz, 1H), 9.14 (s, 1H), 8.08 (q, J = 8.3 Hz, 1H), 7.89 (s, 2H), 7.54 (d, J = 7.5 Hz, 1H), 6.98- 6.88 (m, 1H), 5.73 (dq, J = 16.3, 5.2 Hz, 1H), 5.29-5.02 (m, 2H), 4.94 (d, J = 17.2 Hz, 1H), 4.66 (s, 2H), 3.36 (d, J = 11.0 Hz, 2H), 3.17 (s, 2H), 2.81 (d, J = 16.3 Hz, 3H), 2.58 (s, 6H), 2.25 (d, J = 12.0 Hz, 1H), 2.15-1.97 (m, 2H), 1.83 (q, J = 11.0 Hz, 1H). |
| 200 | 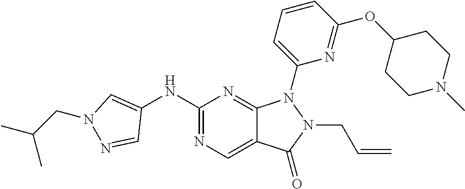 [SMILES: C=CCn1c(=O)c2cnc(Nc3cnn(CC(C)C)c3)nc2n1-c1cccc(OC2CCN(C)CC2)n1] | 504.3 | 1H NMR (400 MHz, dmso) δ 10.33 (s, 1H), 8.79 (s, 1H), 8.04-7.79 (m, 2H), 7.53 (s, 1H), 7.42 (t, J = 8.3 Hz, 1H), 6.81 (d, J = 8.2 Hz, 1H), 5.70 (ddt, J = 16.2, 10.6, 5.8 Hz, 1H), 5.05 (d, J = 10.2 Hz, 1H), 4.99-4.85 (m, 2H), 4.55 (d, J = 5.4 Hz, 2H), 3.87 (d, J = 7.1 Hz, 2H), 2.60 (s, 2H), 2.16 (s, 6H), 2.03 (dt, J = 13.5, 6.7 Hz, 1H), 1.92 (s, 2H), 1.66 (q, J = 8.9 Hz, 2H), 0.82 (d, J = 6.7 Hz, 6H). |
| 201 | 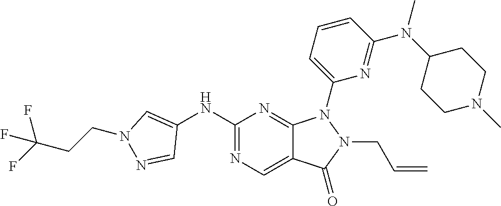 [SMILES: C=CCn1c(=O)c2cnc(Nc3cnn(CCC(F)(F)F)c3)nc2n1-c1cccc(N(C)C2CCN(C)CC2)n1] | 557.0 | 1H NMR (400 MHz, dmso) δ 10.31 (s, 1H), 8.78 (s, 1H), 7.97 (s, 1H), 7.72 (d, J = 8.1 Hz, 1H), 7.62 (s, 1H), 7.01 (d, J = 7.7 Hz, 1H), 6.61 (d, J = 8.4 Hz, 1H), 5.69 (d, J = 6.3 Hz, 1H), 5.03 (d, J = 10.6 Hz, 1H), 4.88 (d, J = 17.8 Hz, 1H), 4.61 (d, J = 5.8 Hz, 2H), 4.34 (t, J = 6.7 Hz, 2H), 2.85 (s, 7H), 2.54 (s, 1H), 2.17 (s, 3H), 2.01-1.69 (m, 4H), 1.51 (d, J = 10.3 Hz, 2H). |
| 202 | 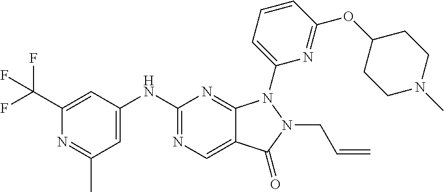 [SMILES: C=CCn1c(=O)c2cnc(Nc3cc(C)nc(C(F)(F)F)c3)nc2n1-c1cccc(OC2CCN(C)CC2)n1] | 541.0 | 1H NMR (500 MHz, DMSO) δ 10.95 (s, 1H), 9.68 (d, J = 39.7 Hz, 1H), 9.05 (s, 1H), 8.16-8.07 (m, 1H), 7.99 (dt, J = 12.6, 7.9 Hz, 1H), 7.83 (s, 1H), 7.48 (dd, J = 7.5, 5.0 Hz, 1H), 6.92 (t, J = 8.2 Hz, 1H), 5.79-5.65 (m, 1H), 5.33-5.02 (m, 2H), 4.93 (d, J = 17.2 Hz, 1H), 4.62 (s, 2H), 3.49 (d, J = 12.1 Hz, 1H), 3.35 (d, J = 11.5 Hz, 1H), 3.17 (q, J = 10.0 Hz, 2H), 2.81 (dd, J = 18.3, 4.3 Hz, 3H), 2.49 (d, J = 2.0 Hz, 3H), 2.26 (d, J = 11.4 Hz, 1H), 2.06 (dt, J = 37.0, 14.4 |
| | | | Hz, 2H), 1.81 (qd, J = 13.8, 3.8 Hz, 1H). |
| 204 | 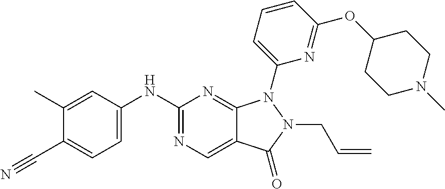 [SMILES: C=CCn1c(=O)c2cnc(Nc3ccc(C#N)c(C)c3)nc2n1-c1cccc(OC2CCN(C)CC2)n1] | 497.0 | 1H NMR (400 MHz, dmso) δ 10.70 (s, 1H), 9.81 (d, J = 37.9 Hz, 1H), 8.98 (s, 1H), 8.05 (q, J = 8.2 Hz, 1H), 7.98 (s, 1H), 7.68 (q, J = 8.7 Hz, 2H), 7.50 (d, J = 7.5 Hz, 1H), 6.88 (t, J = 7.2 Hz, 1H), 5.79-5.63 (m, 1H), 5.33-5.01 (m, 2H), 4.93 (d, J = 17.1 Hz, 1H), 4.63 (s, 2H), 3.50 (s, 1H), 3.35 (d, J = 13.7 Hz, 1H), 3.25-3.07 (m, 2H), 2.81 (d, J = 12.1 Hz, 3H), 2.44 (s, 3H), 2.26 (d, J = 12.9 Hz, 1H), 2.17-1.97 (m, 2H), 1.81 (q, J = 11.0 Hz, 1H). |
| 205 | 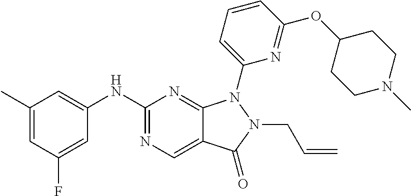 | 490.0 | 1H NMR (400 MHz, dmso) δ 10.44 (s, 1H), 9.87 (d, J = 42.7 Hz, 1H), 8.93 (s, 1H), 8.03-7.89 (m, 1H), 7.58-7.44 (m, 2H), 7.42 (s, 1H), 6.87 (t, J = 8.6 Hz, 1H), 6.68 (d, J = 9.3 Hz, 1H), 5.79- 5.63 (m, 1H), 5.29-5.01 (m, 2H), 4.93 (d, J = 17.1 Hz, 1H), 4.61 (t, J = 4.3 Hz, 2H), 3.49 (d, J = 11.8 Hz, 1H), 3.35 (d, J = 11.6 Hz, 1H), 3.17 (q, J = 11.5 Hz, 2H), 2.81 (dd, J = 13.4, 3.7 Hz, 3H), 2.29 (s, 4H), 2.18-1.97 (m, 2H), 1.82 (q, J = 9.9 Hz, 1H). |
| 206 | 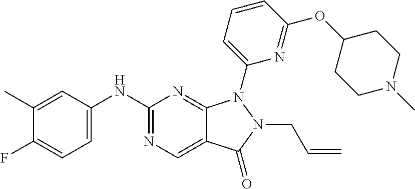 | 490.0 | 1H NMR (400 MHz, dmso) δ 10.31 (s, 1H), 9.70 (d, J = 38.1 Hz, 1H), 8.88 (s, 1H), 7.99 (q, J = 8.2 Hz, 1H), 7.74 (s, 1H), 7.48 (d, J = 8.0 Hz, 2H), 7.10 (t, J = 9.2 Hz, 1H), 6.84 (t, J = 8.2 Hz, 1H), 5.78-5.62 (m, 1H), 5.31-5.01 (m, 2H), 4.93 (d, J = 17.1 Hz, 1H), 4.68- 4.53 (m, 2H), 3.49 (d, J = 11.5 Hz, 1H), 3.35 (d, J = 11.7 Hz, 1H), 3.17 (s, 2H), 2.88-2.72 (m, 3H), 2.29-2.17 (m, 4H), 2.15-1.96 (m, 2H), 1.80 (q, J = 10.7 Hz, 1H). |
| 207 | 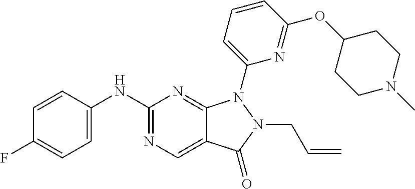 [SMILES: C=CCn1c(=O)c2cnc(Nc3ccc(F)cc3)nc2n1-c1cccc(OC2CCN(C)CC2)n1] | 476.0 | 1H NMR (400 MHz, dmso) δ 10.35 (s, 1H), 9.70 (d, J = 36.2 Hz, 1H), 8.89 (s, 1H), 8.02 (q, J = 7.7 Hz, 1H), 7.74 (s, 2H), 7.47 (d, J = 7.6 Hz, 1H), 7.18 (t, J = 8.5 Hz, 2H), 6.83 (t, J = 8.1 Hz, 1H), 5.70 (ddq, J = 14.7, 10.3, 5.3 Hz, 1H), 5.36-4.99 (m, 2H), 4.93 (d, J = 17.1 Hz, 1H), 4.61 (s, 2H), 3.49 (d, J = 11.1 Hz, 1H), 3.33 (s, 1H), 3.17 (s, 2H), 2.81 (d, J = 9.3 Hz, 3H), 2.26 (d, J = 11.3 Hz, 1H), 2.04 (dd, J = 27.2, 14.9 Hz, 2H), 1.80 (q, J = 11.0 Hz, 1H). |
| 208 | 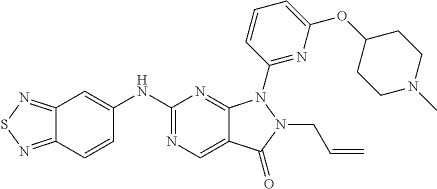 [SMILES: C=CCn1c(=O)c2cnc(Nc3ccc4nsnc4c3)nc2n1-c1cccc(OC2CCN(C)CC2)n1] | 516.0 | 1H NMR (400 MHz, dmso) δ 10.83 (s, 1H), 9.48-9.28 (m, 1H), 9.05 (s, 1H), 8.78 (s, 1H), 8.13-7.98 (m, 2H), 7.91 (d, J = 9.4 Hz, 1H), 7.56 (d, J = 6.5 Hz, 1H), 6.98-6.85 (m, 1H), 5.82-5.64 (m, 1H), 5.35-5.03 (m, 2H), 4.95 (d, J = 17.2 Hz, 1H), 4.70-4.57 (m, 2H), 3.48 (d, J = 12.4 Hz, 1H), 3.34 (d, J = 11.5 Hz, 1H), 3.17 (q, J = 10.3 Hz, 2H), 2.80 (dd, J = 14.2, 4.5 Hz, 3H), 2.29 (d, J = 11.9 Hz, 1H), 2.14 (d, J = 13.3 Hz, 1H), 2.01 (t, J = 13.4 Hz, 1H), 1.79 (q, |
| | | | J = 11.2 Hz, 1H). |
| 209 | 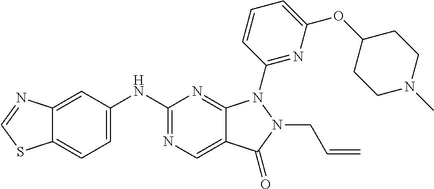 [SMILES: C=CCn1c(=O)c2cnc(Nc3ccc4scnc4c3)nc2n1-c1cccc(OC2CCN(C)CC2)n1] | 415.05 | 1H-NMR (400 MHz, DMSO-d6): δ 10.54 (bs, 1H), 9.39(s, 1H), 8.95 (s, 1H), 8.68 (s, 1H), 8.16 (s, 1H), 8.09-8.07 (d, J = 8.8 Hz, 1H), 7.97-7.93 (t, J = 8 Hz, 1H), 7.79-7.76 (m, 1H), 7.52-7.50 (d, J = 7.6 Hz, 1H), 6.83-6.81 (d, J = 8.4 Hz, 1H), 5.78-5.68 (m, 1H), 5.08-5.05 (d, J = 10 Hz, 1H), 4.97-4.93 (m, 2H), 4.64 (s, 2H), 5.78-5.68 (m, 1H), 5.08- 5.05 (d, J = 10 Hz, 1H), 4.97-4.93 (m, 2H), 4.64-4.62 (d, J = 5.6 Hz, 2H), 2.70-2.64(m, 2H), 2.33-2.03 |
| | | | (m,5 H), 1.98-1.94 (m, |
| | | | 2H), 1.74-1.65 (m, 2H). |
| 210 | 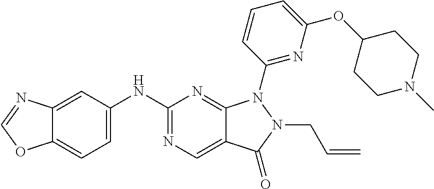 [SMILES: C=CCn1c(=O)c2cnc(Nc3ccc4ocnc4c3)nc2n1-c1cccc(OC2CCN(C)CC2)n1] | Cpd was not made | |
| 211 | 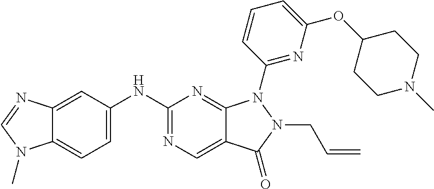 [SMILES: C=CCn1c(=O)c2cnc(Nc3ccc4c(c3)ncn4C)nc2n1-c1cccc(OC2CCN(C)CC2)n1] | 512.1 | 1H-NMR (400 MHz, DMSO-d6): δ 10.31 (s, 1H), 8.88 (s, 1H), 8.14 (d, J = 6.00 Hz, 2H), 7.92 (t, J = 7.60 Hz, 1H), 7.55-7.49 (m, 3H), 6.79 (d, J = 8.40 Hz, 1H), 5.75-5.68 (m, 1H), 5.06 (dd, J = 1.20, 10.20 Hz, 1H), 4.97-4.62 (m, 2H), 4.63-4.62 (m, 2H), 3.83 (s, 3H), 2.68- 2.59 (m, 2H), 2.17-2.14 (m, 5H), 1.95 (t, J = 9.20 Hz, 2H), 1.71-1.67 (m, 2H) |
| 212 | 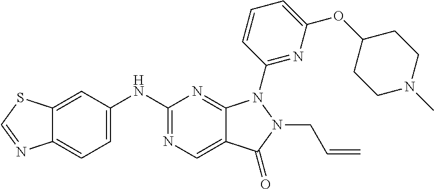 [SMILES: C=CCn1c(=O)c2cnc(Nc3ccc4ncsc4c3)nc2n1-c1cccc(OC2CCN(C)CC2)n1] | 515.0 | 1H NMR (400 MHz, dmso) δ 10.62 (s, 1H), 9.47-9.31 (m, 1H), 9.29 (s, 1H), 8.95 (s, 1H), 8.72 (s, 1H), 8.10-7.94 (m, 2H), 7.74 (d, J = 8.9 Hz, 1H), 7.56 (dd, J = 7.5, 2.7 Hz, 1H), 6.92-6.83 (m, 1H), 5.82-5.63 (m, 1H), 5.30- 5.03 (m, 2H), 4.94 (d, J = 17.2 Hz, 1H), 4.69-4.57 (m, 2H), 3.48 (d, J = 10.9 Hz, 1H), 3.34 (d, J = 11.7 Hz, 1H), 3.22- 3.10 (m, 2H), 2.80 (dd, J = 11.6, 4.6 Hz, 3H), 2.27 (d, J = 12.4 Hz, 1H), 2.16-1.94 (m, 2H), 1.78 (q, J = 10.5 Hz, |
| | | | 1H). |
| 213 | 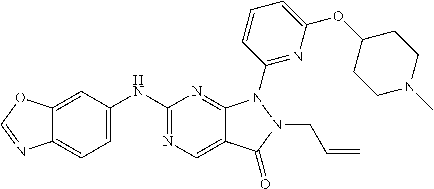 [SMILES: C=CCn1c(=O)c2cnc(Nc3ccc4ncoc4c3)nc2n1-c1cccc(OC2CCN(C)CC2)n1] | Cpd was not made | |
| 214 | 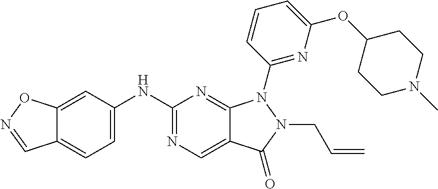 [SMILES: C=CCn1c(=O)c2cnc(Nc3ccc4cnoc4c3)nc2n1-c1cccc(OC2CCN(C)CC2)n1] | 499.0 | 1H NMR (400 MHz, dmso) δ 10.55 (s, 1H), 8.96 (s, 1H), 7.95 (t, J = 7.9 Hz, 1H), 7.51 (d, J = 8.6 Hz, 1H), 7.46 (d, J = 7.6 Hz, 2H), 7.40 (d, J = 8.4 Hz, 1H), 6.80 (d, J = 8.2 Hz, 1H), 5.81-5.64 (m, 1H), 5.06 (d, J = 10.2 Hz, 1H), 5.00- 4.87 (m, 2H), 4.61 (d, J = 5.4 Hz, 2H), 2.63-2.56 (m, 2H), 2.23-2.07 (m, 6H), 1.98-1.87 (m, 2H), 1.66 (q, J = 8.7 Hz, 2H). |
| 215 | 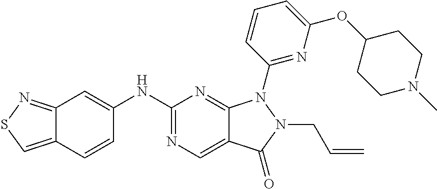 [SMILES: C=CCn1c(=O)c2cnc(Nc3ccc4csnc4c3)nc2n1-c1cccc(OC2CCN(C)CC2)n1] | Cpd was not made | |
| 216 | 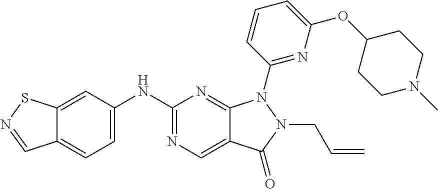 [SMILES: C=CCn1c(=O)c2cnc(Nc3ccc4cnsc4c3)nc2n1-c1cccc(OC2CCN(C)CC2)n1] | Cpd was not made | |
| 217 | 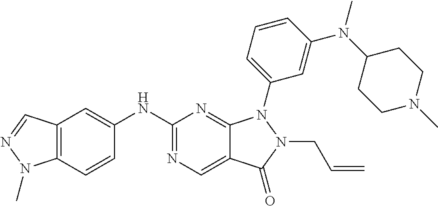 [SMILES: C=CCn1c(=O)c2cnc(Nc3ccc4c(cnn4C)c3)nc2n1-c1cccc(N(C)C2CCN(C)CC2)c1] | 524.1 | 1H-NMR (400 MHz, DMSO-d6): δ 10.26 (s, 1H), 8.86 (s, 1H), 8.55 (s, 1H), 8.33 (s, 1H), 7.96 (s, 1H), 7.60-7.53 (m, 2H), 7.36-7.32 (m, 1H), 6.75-6.68 (m, 3H), 5.99-5.97 (m, 1H), 5.75-5.68 (m, 1H), 5.12 (dd, J = 1.20, 10.40 Hz, 1H), 4.97 (dd, J = 1.20, 17.20 Hz, 1H), 4.30 (s, 2H), 4.02 (s, 3H), 3.58-3.50 (m, 3H), 3.11-3.08 (m, 6H), 2.34-2.33 (m, 2H), 1.79-1.74 (m, 2H) |
| 218 | 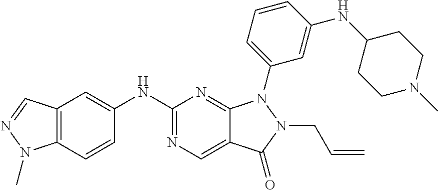 [SMILES: C=CCn1c(=O)c2cnc(Nc3ccc4c(cnn4C)c3)nc2n1-c1cccc(NC2CCN(C)CC2)c1] | 511.6 | 1H-NMR (400 MHz, DMSO-d6): δ 10.30 (s, 1H), 8.86 (s, 1H), 8.24 (s, 1H), 7.93 (s, 1H), 7.55-7.53 (m, 1H), 7.51- 7.48 (m, 1H), 7.09-7.03 (m, 3H), 5.74- 5.65 (m, 1H), 5.10 (dd, J = 1.20, 10.20 Hz, 1H), 4.96 (dd, J = 1.60, 17.00 Hz, 1H), 4.42 (m, 1H), 4.30 (m, 2H), 4.02 (s, 3H), 2.61-2.58 (m, 2H), 2.17-2.14 (m, 5H), 1.91 (m, 2H), 1.67-1.63 (m, 2H). |
| 219 | 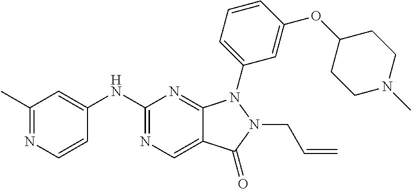 | 472.47 | 1H-NMR (400 MHz, DMSO-d6): δ 10.49 (s, 1H), 8.97 (s, 1H), 8.21 (d, J = 5.60 Hz, 1H), 7.69 (d, J = 2.00 Hz, 1H), 7.51-7.45 (m, 2H), 7.10-7.07 (m, 3H), 5.74-5.67 (m, 1H), 5.10 (dd, J = 1.20, 10.20 Hz, 1H), 4.95 (dd, J = 1.20, 17.20 Hz, 1H), 4.46 (t, J = 4.00 Hz, 1H), 4.44- 4.32 (m, 2H), 2.68-2.60 (m, 2H), 2.50- 2.37 (m, 3H), 2.33-2.17 (m, 5H), 1.95- 1.93 (m, 2H), 1.69-1.66 (m, 2H) |
| 220 | 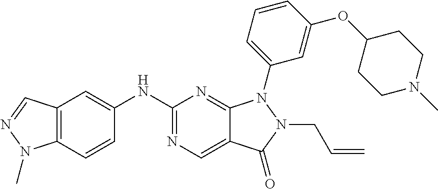 [SMILES: C=CCn1c(=O)c2cnc(Nc3ccc4c(cnn4C)c3)nc2n1-c1cccc(OC2CCN(C)CC2)c1] | 511.6 | 1H-NMR (400 MHz, DMSO-d6): δ 10.30 (s, 1H), 8.86 (s, 1H), 8.24 (s, 1H), 7.93 (s, 1H), 7.55-7.53 (m, 1H), 7.51- 7.48 (m, 1H), 7.09-7.03 (m, 3H), 5.74- 5.65 (m, 1H), 5.10 (dd, J = 1.20, 10.20 Hz, 1H), 4.96 (dd, J = 1.60, 17.00 Hz, 1H), 4.42 (m, 1H), 4.30 (m, 2H), 4.02 (s, 3H), 2.61-2.58 (m, 2H), 2.17-2.14 (m, 5H), 1.91 (m, 2H), 1.67-1.63 (m, 2H). |
| 221 | 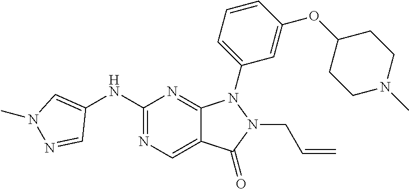 | 461.5 | 1H-NMR (400 MHz, DMSO-d6): δ 9.93 (s, 1H), 8.76 (s, 1H), 7.76 (s, 1H), 7.50- 7.45 (m, 2H), 7.07-7.03 (m, 3H), 5.72- 5.64 (m, 1H), 5.09 (d, J = 10.40 Hz, 1H), 4.95 (d, J = 17.20 Hz, 1H), 4.45 (m, 1H), 4.31-4.29 (m, 2H), 3.77 (s, 3H), 2.60 (m, 2H), 2.17 (m, 5H), 1.94 (m, 2H), 1.66-1.64 (m, 2H). |
| 222 | 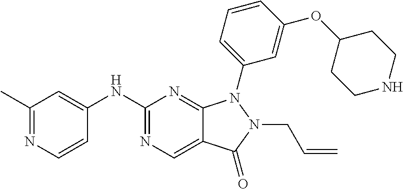 [SMILES: C=CCn1c(=O)c2cnc(Nc3ccnc(C)c3)nc2n1-c1cccc(OC2CCNCC2)c1] | 458.0 | 1H-NMR (400 MHz, DMSO-d6): δ 10.50 (s, 1H), 8.97 (s, 1H), 8.21 (d, J = 5.60 Hz, 1H), 7.70 (s, 1H), 7.51-7.45 (m, 2H), 7.08-7.07 (m, 3H), 5.68-5.67 (m, 1H), 5.10 (dd, J = 1.20, 10.40 Hz, 1H), 4.95 (dd, J = 1.20, 17.20 Hz, 1H), 4.50-4.46 (m, 1H), 4.33-4.32 (m, 2H), 2.96-2.93 (m, 2H), 2.68-2.67 (m, 2H), 2.50 (s, 3H), 1.94-1.88 (m, 5H), 1.50- 1.45 (m, 3H) |
| 223 | 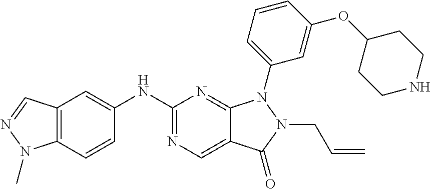 [SMILES: C=CCn1c(=O)c2cnc(Nc3ccc4c(cnn4C)c3)nc2n1-c1cccc(OC2CCNCC2)c1] | 497.4 | 1H-NMR (400 MHz, DMSO-d6): δ 10.29 (s, 1H), 8.86 (s, 1H), 7.93 (s, 1H), 7.62-7.49 (m, 3H), 7.08-7.05 (m, 3H), 5.74-5.68 (m, 1H), 5.10 (d, J = 10.00 Hz, 1H), 4.96 (d, J = 16.80 Hz, 1H), 4.45 (s, 1H), 4.30 (s, 2H), 4.02 (s, 3H), 2.93-2.90 (m, 2H), 1.92-1.89 (m, 2H), 1.76-1.81 (m, 4H), 1.46-1.44 (m, 2H). |
| 224 | 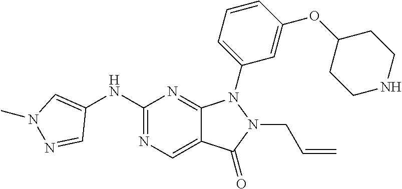 [SMILES: C=CCn1c(=O)c2cnc(Nc3cnn(C)c3)nc2n1-c1cccc(OC2CCNCC2)c1] | 447.0 | 1H-NMR (400 MHz, DMSO-d6): δ 10.28 (s, 1H), 8.78 (s, 1H), 8.30 (s, 1H), 7.78 (s, 1H), 7.51-7.48 (m, 2H), 7.10- 6.99 (m, 3H), 5.73-5.66 (m, 1H), 5.09 (d, J = 10.00 Hz, 1H), 4.95 (d, J = 17.60 Hz, 1H), 4.54 (s, 1H), 4.31-4.22 (m, 2H), 3.78 (s, 3H), 3.02 (m, 2H), 2.51 (m, 2H), 1.96-1.91 (m, 2H), 1.54 (m, 2H). |
| 225 | 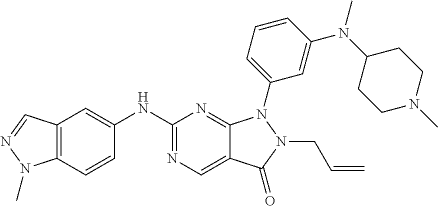 [SMILES: C=CCn1c(=O)c2cnc(Nc3ccc4c(cnn4C)c3)nc2n1-c1cccc(N(C)C2CCN(C)CC2)c1] | 524.1 | 1H-NMR (400 MHz, DMSO-d6): δ 10.26 (s, 1H), 8.86 (s, 1H), 8.55 (s, 1H), 8.33 (s, 1H), 7.96 (s, 1H), 7.60-7.53 (m, 2H), 7.36-7.32 (m, 1H), 6.75-6.68 (m, 3H), 5.99-5.97 (m, 1H), 5.75-5.68 (m, 1H), 5.12 (dd, J = 1.20, 10.40 Hz, 1H), 4.97 (dd, J = 1.20, 17.20 Hz, 1H), 4.30 (s, 2H), 4.02 (s, 3H), 3.58-3.50 (m, 3H), 3.11-3.08 (m, 6H), 2.34-2.33 (m, 2H), 1.79-1.74 (m, 2H). |
| 226 | 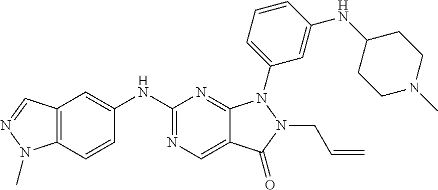 [SMILES: C=CCn1c(=O)c2cnc(Nc3ccc4c(cnn4C)c3)nc2n1-c1cccc(NC2CCN(C)CC2)c1] | 510.6 | 1H-NMR (400 MHz, DMSO-d6): δ 10.23 (s, 1H), 8.87-8.84 (m, 1H), 8.31 (s, 1H), 7.94 (s, 1H), 7.61-7.52 (m, 2H), 7.26 (t, J = 8.00 Hz, 1H), 6.67-6.59 (m, 3H), 5.77-5.87 (m, 1H), 5.74-5.67 (m, 1H), 5.11 (dd, J = 1.20, 10.40 Hz, 1H), 4.96 (dd, J = 1.20, 17.20 Hz, 1H), 4.28 (s, 2H), 4.01 (s, 3H), 3.20-3.15 (m, 1H), 2.71-2.68 (m, 2H), 2.15 (s, 3H), 2.00- 1.87 (m, 4H), 1.42-1.39 (m, 2H). |
| 227 | 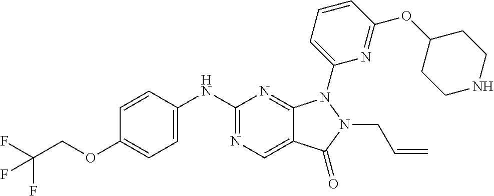 [SMILES: C=CCn1c(=O)c2cnc(Nc3ccc(OCC(F)(F)F)cc3)nc2n1-c1cccc(OC2CCNCC2)n1] | 542.0 | 1H NMR 1H NMR (400 MHz, dmso) δ 10.4 (s, 1H), 8.86 (s, 1H), 8.23 (s, 1H), 7.99 (s, 1H), 7.67 (d, J = 8.4 Hz, 2H), 7.44 (d, J = 7.7 Hz, 1H), 7.04 (d, J = 9.0 Hz, 2H), 6.80 (d, J = 8.2 Hz, 1H), 5.70 (dd, J = 17.1, 10.2 Hz, 1H), 5.05 (d, J = 10.2 Hz, 2H), 4.93 (d, J = 17.4 Hz, 1H), 4.73 (q, J = 8.9 Hz, 2H), 4.61 (m, 2H), 3.07 (s, 2H), 2.85 (m, 2H), 2.00 (m, 2H), 1.69 (m, 2H). |
| 228 | 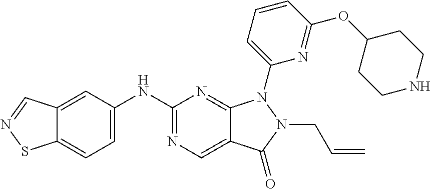 [SMILES: C=CCn1c(=O)c2cnc(Nc3ccc4sncc4c3)nc2n1-c1cccc(OC2CCNCC2)n1] | 501.0 | 1H NMR (400 MHz, cd3od) δ 8.89 (d, J = 10.8 Hz, 2H), 8.72 (s, 1H), 8.07-7.97 (m, 2H), 7.76 (d, J = 10.9 Hz, 1H), 7.55 (d, J = 7.6 Hz, 1H), 6.91 (d, J = 8.2 Hz, 1H), 5.87-5.69 (m, 1H), 5.32 (s, 1H), 5.15-5.00 (m, 2H), 4.71 (d, J = 6.0 Hz, 2H), 3.5-3.1 (m, 4H), 2.12 (m, 4H). |
| 229 | 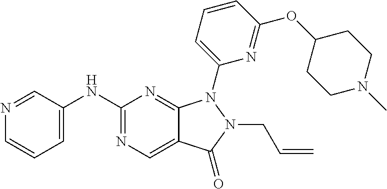 [SMILES: C=CCn1c(=O)c2cnc(Nc3cccnc3)nc2n1-c1cccc(OC2CCN(C)CC2)n1] | 459.0 | 1H NMR (400 MHz, dmso) δ 10.63 (s, 1H), 9.45 (d, J = 20.9 Hz, 1H), 8.99 (d, J = 11.0 Hz, 2H), 8.31 (dd, J = 21.5, 6.3 Hz, 2H), 8.01 (q, J = 8.2 Hz, 1H), 7.59- 7.50 (m, 1H), 7.46 (d, J = 5.7 Hz, 1H), 6.90-6.81 (m, 1H), 5.78-5.64 (m, 1H), 5.30-5.03 (m, 2H), 4.93 (d, J = 17.2 Hz, 1H), 4.65-4.56 (m, 2H), 3.49 (d, J = 11.9 Hz, 1H), 3.35 (d, J = 11.6 Hz, 1H), 3.25-3.09 (m, 2H), 2.81 (dd, J = 13.8, 4.4 Hz, 3H), 2.27 (d, J = 12.1 Hz, 1H), 2.17-1.93 (m, 2H), 1.78 (q, |
| | | | J = 10.4 Hz, 1H). |
| 230 | 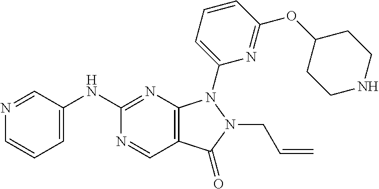 [SMILES: C=CCn1c(=O)c2cnc(Nc3cccnc3)nc2n1-c1cccc(OC2CCNCC2)n1] | 445.0 | 1H NMR (400 MHz, dmso) δ 10.44 (s, 1H), 8.94 (s, 1H), 8.86 (d, J = 2.5 Hz, 1H), 8.24 (dd, J = 4.7, 1.4 Hz, 1H), 8.22- 8.14 (m, 1H), 7.93 (t, J = 7.9 Hz, 1H), 7.44-7.31 (m, 2H), 6.80 (d, J = 8.2 Hz, 1H), 5.79-5.64 (m, 1H), 5.05 (d, J = 10.4 Hz, 1H), 5.01-4.88 (m, 2H), 4.59 (d, J = 5.6 Hz, 2H), 2.99-2.88 (m, 2H), 2.57-2.52 (m, 3H), 1.97-1.85 (m, 2H), 1.55-1.41 (m, 2H). |
| 231 | 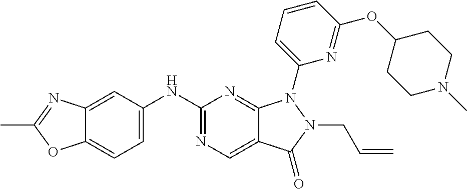 | 513.0 | 1H NMR (400 MHz, dmso) δ 10.45 (s, 1H), 9.44 (d, J = 23.9 Hz, 1H), 8.91 (s, 1H), 8.15 (s, 1H), 7.97 (q, J = 8.1 Hz, 1H), 7.65-7.55 (m, 2H), 7.50 (d, J = 7.6 Hz, 1H), 6.86 (t, J = 7.7 Hz, 1H), 5.81-5.63 (m, 1H), 5.32-5.02 (m, 2H), 4.94 (d, J = 17.2 Hz, 1H), 4.66- 4.55 (m, 2H), 3.49 (d, J = 11.6 Hz, 1H), 3.34 (d, J = 11.5 Hz, 1H), 3.25-3.10 (m, 2H), 2.81 (dd, J = 12.9, 4.5 Hz, 3H), 2.60 (s, 3H), 2.29 (d, J = 11.9 Hz, 1H), 2.18-1.95 (m, 2H), 1.79 (q, J = 10.5 |
| | | | Hz, 1H). |
| 232 | 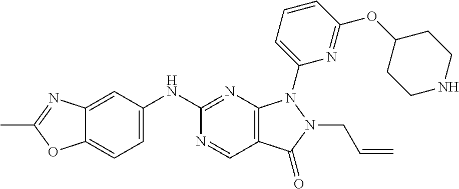 [SMILES: C=CCn1c(=O)c2cnc(Nc3ccc4oc(C)nc4c3)nc2n1-c1cccc(OC2CCNCC2)n1] | 499.0 | 1H NMR (400 MHz, dmso) δ 10.44 (s, 1H), 8.91 (s, 1H), 8.45 (d, J = 24.6 Hz, 2H), 8.15 (s, 1H), 7.96 (t, J = 7.9 Hz, 1H), 7.64-7.55 (m, 2H), 7.49 (d, J = 7.7 Hz, 1H), 6.87 (d, J = 8.1 Hz, 1H), 5.78-5.64 (m, 1H), 5.25-5.13 (m, 1H), 5.06 (d, J = 10.3 Hz, 1H), 4.94 (d, J = 17.2 Hz, 1H), 4.60 (d, J = 4.6 Hz, 2H), 3.29-3.19 (m, 2H), 3.18-3.06 (m, 2H), 2.60 (s, 3H), 2.17-2.05 (m, 2H), 1.93-1.80 (m, 2H). |
| 233 | 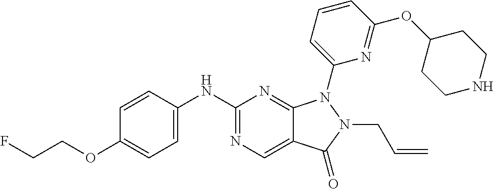 [SMILES: C=CCn1c(=O)c2cnc(Nc3ccc(OCCF)cc3)nc2n1-c1cccc(OC2CCNCC2)n1] | 506.53 | 1H-NMR (400 MHz, DMSO-d6): δ 10.20 (br s, 1H), 8.85 (s, 1H), 7.95 (t, J = 7.60 Hz, 1H), 7.64 (d, J = 8.40 Hz, 2H), 7.43 (d, J = 8.00 Hz, 1H), 6.96 (d, J = 8.80 Hz, 2H), 6.77 (d, J = 8.00 Hz, 1H), 5.70-5.66 (m, 1H), 5.06 (d, J = 11.20 Hz, 1H), 5.01-4.98 (m, 2H), 4.97- 4.91 (m, 1H), 4.81-4.79 (m, 1H), 4.69- 4.61 (m, 2H), 4.27-4.25 (m, 1H), 4.19- 4.18 (m, 1H), 2.96-2.93 (m, 2H), 2.58- 2.51 (m, 2H), 1.92 (d, J = 8.40 Hz, 2H), 1.50 (d, J = 9.60 Hz, 2H) |
| 234 | 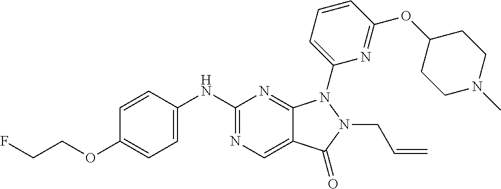 [SMILES: C=CCn1c(=O)c2cnc(Nc3ccc(OCCF)cc3)nc2n1-c1cccc(OC2CCN(C)CC2)n1] | 520.4 | 1H-NMR (400 MHz, DMSO-d6): δ 10.21 (br s, 1H), 8.85 (s, 1H), 7.96 (t, J = 8.00 Hz, 1H), 7.64 (d, J = 8.40 Hz, 2H), 7.43 (d, J = 7.60 Hz, 1H), 6.96 (d, J = 9.20 Hz, 2H), 6.78 (d, J = 8.40 Hz, 1H), 5.56-5.66 (m, 1H), 5.06 (d, J = 10.40 Hz, 1H), 4.96-4.93 (m, 2H), 4.81- 4.80 (m, 1H), 4.69-4.60 (m, 3H), 4.19- 4.25 (m, 2H), 2.63-2.61 (m, 2H), 2.18- 2.15 (m, 5H), 1.93-1.91 (m, 3H), 1.76- 1.67 (m, 2H) |
| 235 | 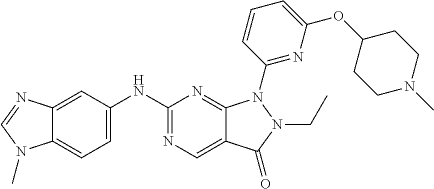 [SMILES: CCn1c(=O)c2cnc(Nc3ccc4c(c3)ncn4C)nc2n1-c1cccc(OC2CCN(C)CC2)n1] | 500.49 | 1H-NMR (400 MHz, DMSO-d6): δ 10.30 (s, 1H), 8.85 (s, 1H), 8.15 (s, 2H), 7.94 (t, J = 8.00 Hz, 1H), 7.57-7.49 (m, 3H), 6.81 (d, J = 8.00 Hz, 1H), 4.95- 4.90 (m, 1H), 4.01 (q, J = 2.40 Hz, 2H), 3.83 (s, 3H), 2.68-2.60 (m, 2H), 2.17- 2.12 (m, 5H), 1.95-1.93 (m, 2H), 1.70- 1.64 (m, 2H), 1.03 (t, J = 6.80 Hz, 3H). |
| 236 | 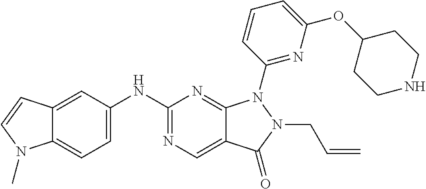 [SMILES: C=CCn1c(=O)c2cnc(Nc3ccc4c(ccn4C)c3)nc2n1-c1cccc(OC2CCNCC2)n1] | 497.48 | 1H-NMR (400 MHz, DMSO-d6,): δ 10.22 (bs, 1H), 8.84 (s, 1H), 8.04 (bs, 1H), 7.93-7.90 (t, J = 15.6 Hz, 1H), 7.51-7.49 (d, J = 7.6 Hz, 1H), 7.38-7.36 (d, J = 8 Hz, 2H), 7.31-7.30 (d, J = 2.8 Hz, 1H), 6.78-6.76 (d, J = 8 Hz, 1H), 6.39-6.38 (d, J = 2.8 Hz, 1H), 5.74-5.68 (m, 1H), 5.07-4.91 (m, 3H), 4.62 (s, 2H), 3.78 (s, 3H), 2.96-2.92 (m, 2H), 2.59-2.55 (m, 2H), 1.93-1.89 (m, 2H), 1.54-1.46 (m, 2H). |
| 237 | 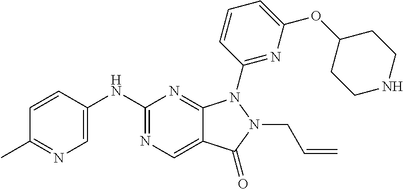 [SMILES: C=CCn1c(=O)c2cnc(Nc3ccc(C)nc3)nc2n1-c1cccc(OC2CCNCC2)n1] | 459.3 | 1H-NMR (400 MHz, DMSO-d6,): δ 10.34 (s, 1H), 8.91 (s, 1H), 8.73 (d, J = 2.40 Hz, 1H), 8.05 (dd, J = 2.40, 8.60 Hz, 1H), 7.93 (t, J = 8.00 Hz, 1H), 7.38 (d, J = 7.60 Hz, 1H), 7.21 (d, J = 8.40 Hz, 1H), 6.80 (d, J = 8.00 Hz, 1H), 5.75- 5.68 (m, 1H), 5.06 (dd, J = 1.20, 10.40 Hz, 1H), 5.00-4.91 (m, 2H), 4.70 (t, J = 5.60 Hz, 2H), 2.97-2.92 (m, 2H), 2.59- 2.56 (m, 2H), 2.43 (s, 3H), 1.94-1.90 (m, 2H), 1.87 (s, 3H), 1.54-1.49 (m, 2H). (1 Acetic acid salt). |
| 238 | 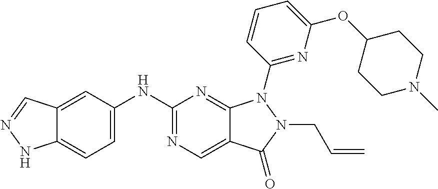 [SMILES: C=CCn1c(=O)c2cnc(Nc3ccc4[nH]ncc4c3)nc2n1-c1cccc(OC2CCN(C)CC2)n1] | 498.51 | 1H-NMR (400 MHz, DMSO-d6): δ 13.00 (s, 1H), 10.35 (s, 1H), 8.88 (s, 1H), 8.26 (s, 1H), 8.05-7.96 (m, 2H), 7.58-7.48 (m, 3H), 6.80-6.78 (m, 1H), 5.75-5.67 (m, 1H), 5.08-4.92 (m, 3H), 4.62-4.61 (m, 2H), 2.68-2.67 (m, 2H), 2.17-2.14 (m, 5H), 1.93-1.76 (m, 4H), 1.72-1.69 (m, 2H) |
| 239 | 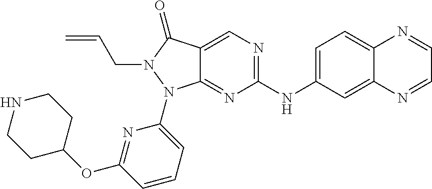 [SMILES: C=CCn1c(=O)c2cnc(Nc3ccc4nccnc4c3)nc2n1-c1cccc(OC2CCNCC2)n1] | 496.0 | 1H NMR (400 MHz, cd3od) δ 11.68 (s, 1H), 9.85 (s, 1H), 9.71 (d, J = 1.8 Hz, 1H), 9.59 (dd, J = 17.3, 2.0 Hz, 1H), 9.26 (d, J = 25.6 Hz, 2H), 9.00-8.73 (m, 3H), 8.40 (d, J = 7.6 Hz, 1H), 7.71 (d, J = 8.2 Hz, 1H), 7.36 (s, 1H), 6.54 (ddd, J = 16.1, 10.4, 5.2 Hz, 1H), 6.02 (s, 1H), 5.88 (d, J = 10.3 Hz, 1H), 5.77 (d, J = 18.3 Hz, 1H), 5.46 (d, J = 5.5 Hz, 2H), 3.99 (d, J = 36.7 Hz, 4H), 2.92 (s, 2H), 2.70 (s, 2H). |
| 240 | 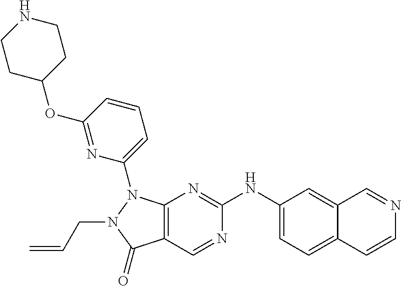 [SMILES: C=CCn1c(=O)c2cnc(Nc3ccc4ccncc4c3)nc2n1-c1cccc(OC2CCNCC2)n1] | 495.0 | 1H NMR 1H NMR (400 MHz, dmso) δ 10.83 (s, 1H), 9.29 (s, 1H), 9.01 (s, 1H), 8.77 (s, 1H), 8.46 (d, J = 5.7 Hz, 2H), 8.18-7.85 (m, 3H), 7.63 (d, J = 7.4 Hz, 1H), 6.92 (2H, d), 5.73 (dd, J = 17.0, 10.4 Hz, 1H), 5.19 (s, 1H), 5.08 (d, J = 10.4 Hz, 1H), 4.95 (d, J = 18.2 Hz, 1H), 4.63 (d, J = 5.6 Hz, 2H), 3.14 (m, 4H), 2.10 (s, 2H), 1.88 (s, 2H). |
| 241 | 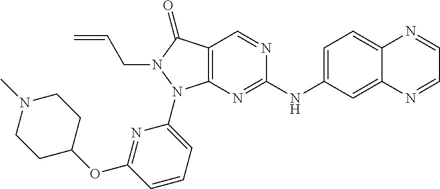 [SMILES: C=CCn1c(=O)c2cnc(Nc3ccc4nccnc4c3)nc2n1-c1cccc(OC2CCN(C)CC2)n1] | 510.0 | 1H NMR: 1H NMR (400 MHz, dmso) δ 10.85 (s, 1H), 9.03 (s, 1H), 8.89 (s, 1H), 8.78 (d, J = 15.5 Hz, 2H), 8.23- 7.92 (m, 3H), 7.55 (d, J = 7.6 Hz, 1H), 6.84 (d, J = 8.2 Hz, 1H), 5.82-5.66 (m, 1H), 5.07 (d, J = 10.3 Hz, 1H), 4.95 (d, J = 17.0 Hz, 2H), 4.65 (d, J = 5.5 Hz, 2H), 2.58 (s, 2H), 2.16 (s, 3H), 1.93 (s, 2H), 1.67 (d, J = 9.1 Hz, 2H), 1.26 (d, J = 26.2 Hz, 2H). |
| 242 | 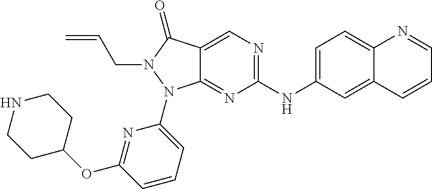 [SMILES: C=CCn1c(=O)c2cnc(Nc3ccc4ncccc4c3)nc2n1-c1cccc(OC2CCNCC2)n1] | 495.0 | 1H NMR 1H NMR (400 MHz, dmso) δ 10.71 (s, 1H), 8.99 (s, 1H), 8.80 (s, 1H), 8.54 (s, 3H), 8.22 (d, J = 8.2 Hz, 1H), 8.10 (s, 1H), 7.98 (s, 2H), 7.71-7.46 (m, 2H), 6.90 (d, J = 8.2 Hz, 1H), 5.72 (s, 1H), 5.19 (s, 1H), 5.07 (d, J = 10.3 Hz, 1H), 4.95 (d, J = 17.2 Hz, 1H), 4.63 (d, J = 5.5 Hz, 2H), 3.14 (m, 4H), 2.10 (s, 2H), 1.87 (s, 2H). |
| 243 | 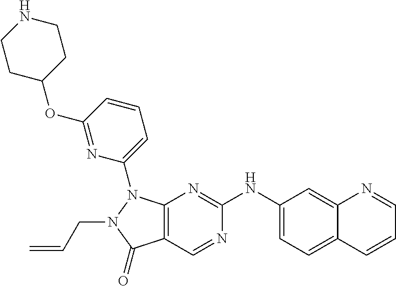 [SMILES: C=CCn1c(=O)c2cnc(Nc3ccc4cccnc4c3)nc2n1-c1cccc(OC2CCNCC2)n1] | 495.0 | |
| 244 | 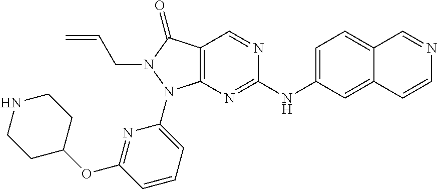 [SMILES: C=CCn1c(=O)c2cnc(Nc3ccc4cnccc4c3)nc2n1-c1cccc(OC2CCNCC2)n1] | 495.0 | 1H NMR 1H NMR (400 MHz, dmso) δ 11.00 (s, 1H), 9.36 (s, 1H), 9.06 (s, 1H), 8.69 (s, 1H), 8.44 (d, J = 30.7 Hz, 3H), 8.30-8.07 (m, 2H), 7.92 (d, J = 47.2 Hz, 2H), 7.63 (d, J = 7.5 Hz, 1H), 6.93 (d, J = 8.2 Hz, 1H), 5.73 (s, 1H), 5.19 (s, 1H), 5.08 (d, J = 10.2 Hz, 1H), 4.95 (d, J = 17.1 Hz, 1H), 4.65 (s, 2H), 3.14 (m, 4H), 2.10 (s, 2H), 1.87 (s, 2H). |
| 245 | 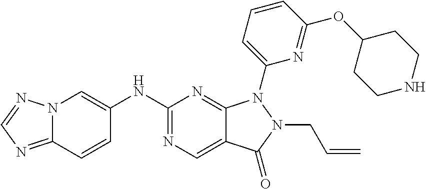 [SMILES: C=CCn1c(=O)c2cnc(Nc3ccc4ncnn4c3)nc2n1-c1cccc(OC2CCNCC2)n1] | 485.0 | 1H NMR (400 MHz, dmso) δ 10.78 (s, 1H), 9.48 (s, 1H), 9.01 (s, 1H), 8.58 (d, J = 16.7 Hz, 2H), 8.29 (s, 1H), 8.17-8.11 (m, 1H), 8.07 (t, J = 7.6 Hz, 1H), 8.01 (d, J = 11.5 Hz, 1H), 7.95 (d, J = 9.7 Hz, 1H), 7.66-7.55 (m, 1H), 6.88 (d, J = 8.2 Hz, 1H), 5.82-5.63 (m, 1H), 5.22- 5.13 (m, 1H), 5.07 (d, J = 10.3 Hz, 1H), 4.93 (d, J = 18.3 Hz, 1H), 4.63 (d, J = 5.4 Hz, 2H), 3.29-3.20 (m, 2H), 3.19- 3.07 (m, 2H), 2.16-2.04 (m, 2H), 1.92- 1.77 (m, 2H). |
| 246 | 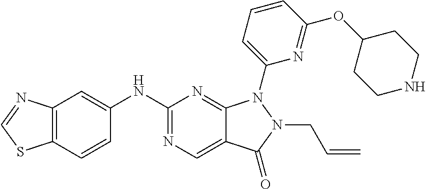 [SMILES: C=CCn1c(=O)c2cnc(Nc3ccc4scnc4c3)nc2n1-c1cccc(OC2CCNCC2)n1] | 501.4 | 1H-NMR (400 MHz, DMSO-d6): δ 10.56 (s, 1H), 9.40 (s, 1H), 8.96 (s, 1H), 8.70 (s, 1H), 8.32 (s, 1H), 8.09 (d, J = 8.80 Hz, 1H), 7.97 (t, J = 8.00 Hz, 1H), 7.78 (dd, J = 2.00, 8.80 Hz, 1H), 7.53 (d, J = 7.60 Hz, 1H), 6.85 (d, J = 8.00 Hz, 1H), 5.77-5.68 (m, 1H), 5.11-5.06 (m, 2H), 4.95 (dd, J = 1.20, 17.20 Hz, 1H), 4.64 (d, J = 5.60 Hz, 2H), 3.07-3.04 (m, 2H), 2.80-2.75 (m, 2H), 2.02-2.00 (m, 2H), 1.70-1.63 (m, 2H), |
| 247 | 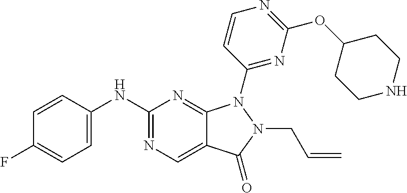 [SMILES: C=CCn1c(=O)c2cnc(Nc3ccc(F)cc3)nc2n1-c1ccnc(OC2CCNCC2)n1] | 463.43 | 1H-NMR (400 MHz, DMSO-d6): δ 10.52 (s, 1H), 8.94 (s, 1H), 8.68 (d, J = 5.60 Hz, 1H), 7.76-7.73 (m, 3H), 7.28- 7.24 (m, 2H), 5.71-5.65 (m, 1H), 5.11- 5.06 (m, 3H), 4.97-4.75 (m, 2H), 3.05- 3.01 (m, 2H), 2.72-2.67 (m, 2H), 2.03- 2.00 (m, 2H), 1.65-1.62 (m, 2H) |
| 248 | 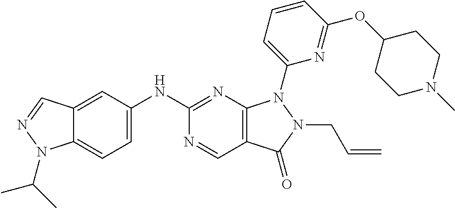 [SMILES: C=CCn1c(=O)c2cnc(Nc3ccc4c(cnn4C(C)C)c3)nc2n1-c1cccc(OC2CCN(C)CC2)n1] | 540.0 | 1H NMR (400 MHz, dmso) δ 10.43 (s, 1H), 9.50 (d, J = 30.4 Hz, 1H), 8.88 (s, 1H), 8.26 (s, 1H), 8.14-7.98 (m, 2H), 7.66 (d, J = 9.1 Hz, 1H), 7.56 (t, J = 7.7 Hz, 2H), 6.84 (t, J = 8.4 Hz, 1H), 5.80- 5.60 (m, 1H), 5.32-5.03 (m, 2H), 5.00- 4.88 (m, 2H), 4.62 (s, 2H), 3.49 (d, J = 12.5 Hz, 1H), 3.34 (d, J = 11.3 Hz, 1H), 3.17 (p, J = 9.8 Hz, 2H), 2.81 (dd, J = 10.2, 4.6 Hz, 3H), 2.26 (d, J = 11.6 Hz, 1H), 2.12 (d, J = 14.0 Hz, 1H), 2.07- 1.96 (m, 1H), 1.79 (q, J = 11.5 Hz, 1H), 1.47 (d, J = 6.6 Hz, 6H). |
| 249 | 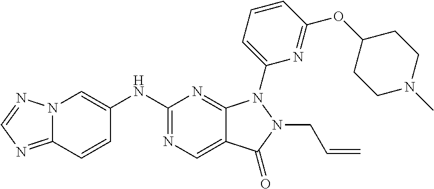 [SMILES: C=CCn1c(=O)c2cnc(Nc3ccc4ncnn4c3)nc2n1-c1cccc(OC2CCN(C)CC2)n1] | 499.0 | 1H NMR (400 MHz, dmso) δ 10.72 (s, 1H), 9.63 (s, 1H), 9.51-9.28 (m, 1H), 9.00 (s, 1H), 8.46 (s, 1H), 8.04 (q, J = 8.1 Hz, 1H), 7.89-7.76 (m, 2H), 7.52 (dd, J = 7.5, 3.4 Hz, 1H), 6.95-6.83 (m, 1H), 5.82-5.65 (m, 1H), 5.33- 5.03 (m, 2H), 4.94 (d, J = 17.3 Hz, 1H), 4.62 (s, 2H), 3.49 (d, J = 12.6 Hz, 1H), 3.34 (d, J = 11.1 Hz, 1H), 3.16 (q, J = 11.0 Hz, 2H), 2.81 (dd, J = 14.4, 4.5 Hz, 3H), 2.29 (d, J = 12.4 Hz, 1H), 2.13 (d, J = 14.9 Hz, 1H), 2.07-1.95 (m, 1H), |
| | | | 1.79 (q, J = 10.0 Hz, 1H). |
| 250 | 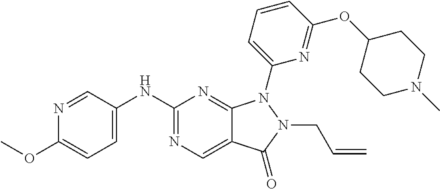 | 489.0 | 1H NMR (400 MHz, dmso) δ 10.30 (s, 1H), 9.49 (d, J = 30.7 Hz, 1H), 8.87 (s, 1H), 8.39 (s, 1H), 8.02 (s, 1H), 7.96 (q, J = 8.2 Hz, 1H), 7.41 (s, 1H), 6.89-6.76 (m, 2H), 5.78-5.61 (m, 1H), 5.29- 5.01 (m, 2H), 4.93 (d, J = 17.3 Hz, 1H), 4.58 (s, 2H), 3.83 (s, 3H), 3.49 (d, J = 11.5 Hz, 1H), 3.33 (d, 1H), 3.25-3.10 (m, 2H), 2.81 (dd, J = 12.7, 4.5 Hz, 3H), 2.27 (d, J = 13.3 Hz, 1H), 2.15-1.97 (m, 2H), 1.87-1.72 (m, 1H). |
| 251 | 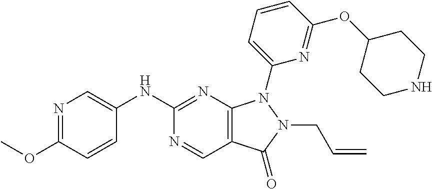 [SMILES: C=CCn1c(=O)c2cnc(Nc3ccc(OC)nc3)nc2n1-c1cccc(OC2CCNCC2)n1] | 475.0 | 1H NMR (400 MHz, dmso) δ 10.40- 9.92 (m, 1H), 8.87 (s, 1H), 8.47 (d, J = 20.6 Hz, 2H), 8.39 (s, 1H), 8.02 (s, 1H), 7.95 (t, J = 7.9 Hz, 1H), 7.41 (s, 1H), 6.83 (d, J = 8.7 Hz, 2H), 5.77-5.60 (m, 1H), 5.25-5.11 (m, 1H), 5.05 (d, J = 10.1 Hz, 1H), 4.92 (d, J = 17.5 Hz, 1H), 4.58 (d, J = 4.5 Hz, 2H), 3.83 (s, 3H), 3.29-3.19 (m, 2H), 3.18-3.08 (m, 2H), 2.17-2.06 (m, 2H), 1.92-1.80 (m, 2H). |
| 252 | 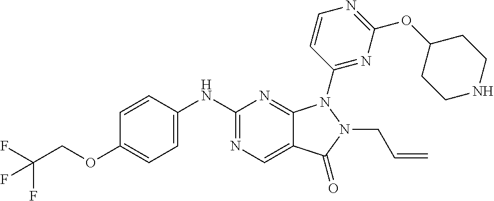 [SMILES: C=CCn1c(=O)c2cnc(Nc3ccc(OCC(F)(F)F)cc3)nc2n1-c1ccnc(OC2CCNCC2)n1] | 543.49 | 1H-NMR (400 MHz, DMSO-d6): δ 10.45 (s, 1H), 8.91 (s, 1H), 8.68 (d, J = 5.20 Hz, 1H), 7.67 (d, J = 8.80 Hz, 2H), 7.12 (d, J = 8.00 Hz, 2H), 5.72-5.65 (m, 1H), 5.08-4.97 (m, 3H), 4.97-4.76 (m, 4H), 2.99-2.97 (m, 2H), 2.68-2.62 (m, 2H), 1.99-1.97 (m, 2H), 1.89-1.76 (m, 2H), 1.57-1.52 (m, 2H) |
| 253 | 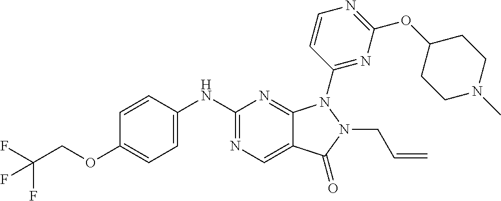 [SMILES: C=CCn1c(=O)c2cnc(Nc3ccc(OCC(F)(F)F)cc3)nc2n1-c1ccnc(OC2CCN(C)CC2)n1] | 557.51 | 1H-NMR (400 MHz, DMSO-d6): δ 10.44 (s, 1H), 8.91 (s, 1H), 8.68 (d, J = 5.20 Hz, 1H), 7.66 (d, J = 9.20 Hz, 3H), 7.12 (d, J = 8.00 Hz, 2H), 5.73-5.66 (m, 1H), 5.08-5.05 (m, 3H), 5.01-4.97 (m, 4H), 2.67-2.65 (m, 2H), 2.16-2.01 (m, 5H), 1.99-1.78 (m, 2H), 1.70-1.72 (m, 2H) |
| 254 | 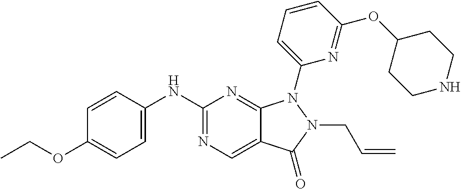 [SMILES: C=CCn1c(=O)c2cnc(Nc3ccc(OCC)cc3)nc2n1-c1cccc(OC2CCNCC2)n1] | 488.5 | 1H-NMR (400 MHz, DMSO-d6): δ 10.18 (s, 1H), 8.84 (t, J = 4.00 Hz, 1H), 7.97-7.93 (m, 1H), 7.61 (d, J = 8.00 Hz, 2H), 7.43 (d, J = 7.60 Hz, 1H), 6.90 (d, J = 9.20 Hz, 2H), 6.78 (d, J = 8.00 Hz, 1H), 5.74-5.66 (m, 1H), 4.91-5.01 (m, 3H), 4.61-4.60 (m, 2H), 4.03-3.98 (m, 2H), 3.00-2.98 (m, 2H), 2.67-2.62 (m, 2H), 1.96-1.90 (m, 5H), 1.58-1.55 (m, 2H), 1.34-1.31 (m, 3H) |
| 255 | 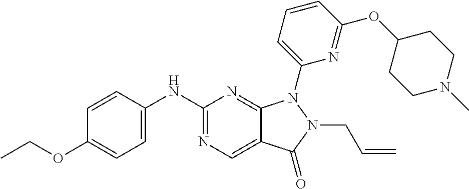 [SMILES: C=CCn1c(=O)c2cnc(Nc3ccc(OCC)cc3)nc2n1-c1cccc(OC2CCN(C)CC2)n1] | 502.53 | 1H-NMR (400 MHz, DMSO-d6): δ 10.18 (s, 1H), 8.84 (s, 1H), 7.95 (t, J = 8.00 Hz, 1H), 7.61 (d, J = 8.40 Hz, 2H), 7.43 (d, J = 7.60 Hz, 1H), 6.90 (d, J = 9.20 Hz, 2H), 6.77 (d, J = 8.40 Hz, 1H), 5.73-5.66 (m, 1H), 5.07-5.04 (m, 1H), 4.96-4.91 (m, 2H), 4.61-4.60 (m, 2H), 4.03-3.98 (m, 2H), 2.62-2.60 (m, 2H), 2.18-2.14 (m, 5H), 1.96-1.93 (m, 2H), 1.72-1.65 (m, 2H), 1.34-1.31 (m, 3H) |
| 256 | 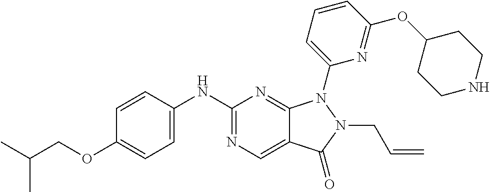 [SMILES: C=CCn1c(=O)c2cnc(Nc3ccc(OCC(C)C)cc3)nc2n1-c1cccc(OC2CCNCC2)n1] | 514.55 | 1H-NMR (400 MHz, DMSO-d6): δ 10.18 (s, 1H), 8.84 (s, 1H), 7.96 (t, J = 8.00 Hz, 1H), 7.62 (d, J = 7.20 Hz, 2H), 7.42 (d, J = 7.60 Hz, 1H), 6.91 (d, J = 9.20 Hz, 2H), 6.77 (d, J = 8.00 Hz, 1H), 5.74-5.67 (m, 1H), 5.07-5.04 (m, 3H), 4.61 (d, J = 4.80 Hz, 2H), 3.73 (d, J = 6.80 Hz, 2H), 2.97-2.94 (m, 2H), 2.60- 2.55 (m, 2H), 2.04-2.01 (m, 1H), 1.99- 1.98 (m, 2H), 1.55-1.47 (m, 2H), 0.99 (d, J = 6.80 Hz, 6H) |
| 257 | 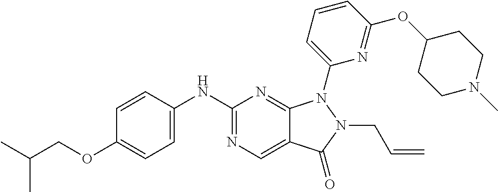 [SMILES: C=CCn1c(=O)c2cnc(Nc3ccc(OCC(C)C)cc3)nc2n1-c1cccc(OC2CCN(C)CC2)n1] | 530.59 | 1H-NMR (400 MHz, DMSO-d6): δ 10.18 (s, 1H), 8.84 (s, 1H), 8.17 (s, 0.3H), 7.96 (t, J = 8.00 Hz, 1H), 7.62 (d, J = 7.60 Hz, 2H), 7.43 (d, J = 7.60 Hz, 2H), 6.91 (d, J = 9.20 Hz, 2H), 6.78 (d, J = 8.00 Hz, 1H), 5.76-5.66 (m, 1H), 5.74-5.67 (m, 1H), 5.06 (d, J = 1.20 Hz, 1H), 4.98-4.92 (m, 2H), 4.60 (d, J = 5.20 Hz, 2H), 3.73 (d, J = 6.80 Hz, 2H), 2.68- 2.64 (m, 2H), 2.33-2.21 (m, 5H), 2.04- 1.95 (m, 3H), 1.73-1.66 (m, 2H), 0.98 (d, J = 6.80 Hz, 6H) |
| 258 | 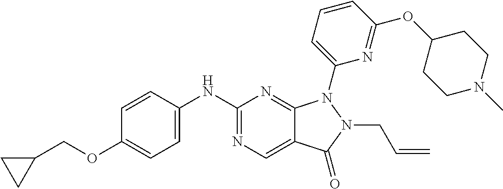 [SMILES: C=CCn1c(=O)c2cnc(Nc3ccc(OCC4CC4)cc3)nc2n1-c1cccc(OC2CCN(C)CC2)n1] | 528.57 | 1H-NMR (400 MHz, DMSO-d6): δ 10.19 (br s, 1H), 8.84 (s, 1H), 7.96 (t, J = 7.60 Hz, 1H), 7.61 (d, J = 8.40 Hz, 2H), 7.42 (d, J = 7.60 Hz, 1H), 6.94 (dd, J = 8.80, 28.40 Hz, 2H), 6.77 (d, J = 8.00 Hz, 1H), 5.72-5.67 (m, 1H), 5.06 (dd, J = 1.20, 10.40 Hz, 1H), 4.96-4.91 (m, 2H), 4.60 (d, J = 4.80 Hz, 2H), 3.80 (d, J = 6.80 Hz, 2H), 2.63-2.60 (m, 2H), 2.18-2.14 (m, 5H), 1.96-1.90 (m, 2H), 1.70-1.66 (m, 2H), 1.22 (s, 1H), 0.60- 0.55 (m, 2H), 0.34-0.31 (m, 2H) |
| 259 | 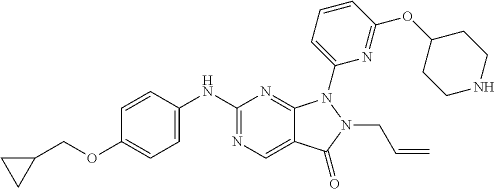 [SMILES: C=CCn1c(=O)c2cnc(Nc3ccc(OCC4CC4)cc3)nc2n1-c1cccc(OC2CCNCC2)n1] | 514.55 | 1H-NMR (400 MHz, DMSO-d6): δ 10.16 (br s, 1H), 8.84 (s, 1H), 7.95 (t, J = 7.60 Hz, 1H), 7.61 (d, J = 8.00 Hz, 2H), 7.42 (d, J = 7.60 Hz, 1H), 6.90 (d, J = 8.80 Hz, 2H), 6.77 (d, J = 8.00 Hz, 1H), 5.72-5.67 (m, 1H), 5.07-4.91 (m, 3H), 4.61-4.60 (m, 2H), 3.81-3.79 (m, 2H), 2.96-2.94 (m, 2H), 2.60-2.58 (m, 3H), 1.94-1.90 (m, 4H), 1.52-1.50 (m, 2H), 1.22-1.20 (m, 1H), 0.60-0.57 (m, 2H), 0.34-0.32 (m, 2H) |
| 260 | 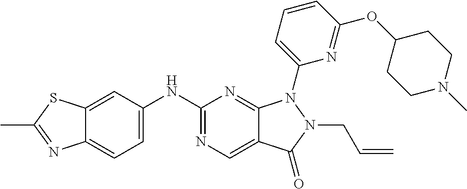 | 529.0 | 1H NMR (400 MHz, dmso) δ 10.56 (s, 1H), 9.44 (d, J = 24.2 Hz, 1H), 8.93 (s, 1H), 8.59 (s, 1H), 7.99 (q, J = 8.1 Hz, 1H), 7.85 (d, J = 8.9 Hz, 1H), 7.65 (d, J = 8.8 Hz, 1H), 7.54 (dd, J = 7.5, 1.9 Hz, 1H), 6.86 (dd, J = 7.9, 6.5 Hz, 1H), 5.80-5.61 (m, 1H), 5.32-5.04 (m, 2H), 4.94 (d, J = 17.3 Hz, 1H), 4.66-4.58 (m, 2H), 3.49 (d, J = 11.9 Hz, 1H), 3.34 (d, J = 12.0 Hz, 1H), 3.24-3.08 (m, 2H), 2.84-2.76 (m, 6H), 2.27 (d, J = 11.4 Hz, 1H), 2.12 (d, J = 15.7 Hz, 1H), |
| | | | 2.07-1.95 (m, 1H), 1.85-1.72 (m, |
| | | | 1H). |
| 261 | 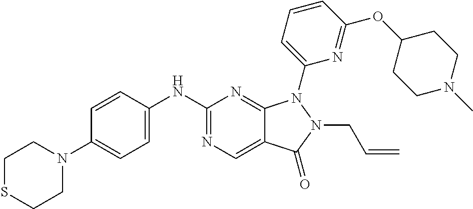 [SMILES: C=CCn1c(=O)c2cnc(Nc3ccc(N4CCSCC4)cc3)nc2n1-c1cccc(OC2CCN(C)CC2)n1] | 559.0 | 1H NMR (400 MHz, dmso) δ 10.20 (s, 1H), 9.34 (s, 1H), 8.83 (s, 1H), 8.01 (s, 1H), 7.58 (s, 2H), 7.49 (d, J = 7.7 Hz, 1H), 6.92 (d, J = 8.9 Hz, 2H), 6.81 (t, J = 8.2 Hz, 1H), 5.70 (ddd, J = 16.4, 10.5, 5.5 Hz, 1H), 5.06 (dd, J = 10.1, 4.9 Hz, 1H), 4.93 (d, J = 17.1 Hz, 2H), 4.60 (s, 2H), 3.48 (s, 3H), 3.16 (s, 2H), 2.81 (dd, J = 11.0, 4.6 Hz, 4H), 2.72- 2.64 (m, 4H), 2.35-1.92 (m, 4H), |
| | | | 1.78 (d, J = 13.2 Hz, 1H). |
| 262 | 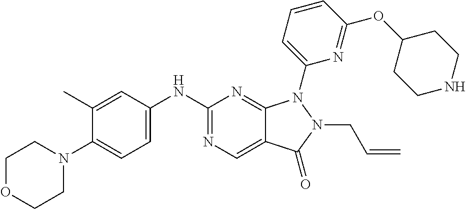 [SMILES: C=CCn1c(=O)c2cnc(Nc3ccc(N4CCOCC4)c(C)c3)nc2n1-c1cccc(OC2CCNCC2)n1] | 543.0 | 1H NMR (400 MHz, dmso) δ 10.25 (s, 1H), 8.85 (s, 1H), 8.56 (d, J = 18.6 Hz, 2H), 7.99 (t, J = 7.9 Hz, 1H), 7.68 (s, 1H), 7.49 (d, J = 7.7 Hz, 1H), 7.43 (d, J = 6.6 Hz, 1H), 7.00 (d, J = 8.7 Hz, 1H), 6.84 (d, J = 8.1 Hz, 1H), 5.70 (ddt, J = 16.3, 10.6, 5.8 Hz, 1H), 5.19 (s, 1H), 5.05 (d, J = 10.2 Hz, 1H), 4.92 (d, J = 17.3 Hz, 1H), 4.60 (d, J = 4.5 Hz, 2H), 4.23 (s, 4H), 3.72 (d, J = 4.5 Hz, 3H), 3.19 (d, J = 34.5 Hz, 4H), 2.84-2.78 (m, 4H), 2.25 (s, 3H), 2.17-2.05 (m, 2H), 1.87 (d, J = 9.2 Hz, 2H) |
| 263 | 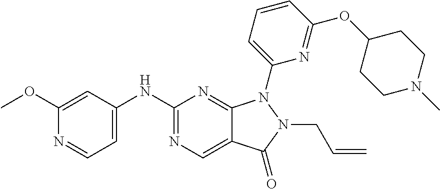 | 489.0 | 1H NMR (400 MHz, dmso) δ 10.68 (s, 1H), 9.47 (d, J = 24.6 Hz, 1H), 9.02 (s, 1H), 8.07-7.96 (m, 2H), 7.47 (d, J = 7.7 Hz, 1H), 7.40 (s, 1H), 7.29 (d, J = 5.4 Hz, 1H), 6.93-6.85 (m, 1H), 5.80- 5.64 (m, 1H), 5.29-5.03 (m, 2H), 4.94 (d, J = 17.3 Hz, 1H), 4.65-4.58 (m, 2H), 3.83 (s, 3H), 3.49 (d, J = 11.6 Hz, 1H), 3.35 (d, J = 11.5 Hz, 1H), 3.23- 3.09 (m, 2H), 2.81 (dd, J = 14.5, 4.6 Hz, 3H), 2.27 (d, J = 11.6 Hz, 1H), 2.12 (d, J = 14.6 Hz, 1H), 2.07-1.95 (m, 1H), |
| | | | 1.79 (q, J = 11.2 Hz, 1H). |
| 264 | 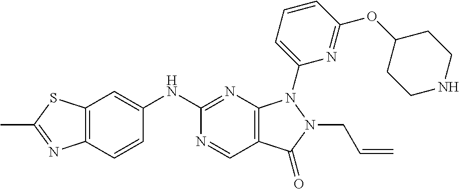 [SMILES: C=CCn1c(=O)c2cnc(Nc3ccc4nc(C)sc4c3)nc2n1-c1cccc(OC2CCNCC2)n1] | 515.0 | 1H NMR (400 MHz, dmso) δ 10.57 (s, 1H), 8.93 (s, 1H), 8.59 (s, 1H), 8.44 (d, J = 28.1 Hz, 2H), 7.99 (t, J = 7.9 Hz, 1H), 7.84 (d, J = 8.8 Hz, 1H), 7.65 (d, J = 8.8 Hz, 1H), 7.54 (d, J = 7.6 Hz, 1H), 6.88 (d, J = 8.2 Hz, 1H), 5.80-5.61 (m, 1H), 5.24-5.15 (m, 1H), 5.06 (d, J = 10.2 Hz, 1H), 4.94 (d, J = 17.0 Hz, 1H), 4.62 (d, J = 4.4 Hz, 2H), 3.30-3.19 (m, 2H), 3.17-3.08 (m, 2H), 2.78 (s, 3H), 2.17- 2.04 (m, 2H), 1.94-1.79 (m, 2H). |
| 265 | 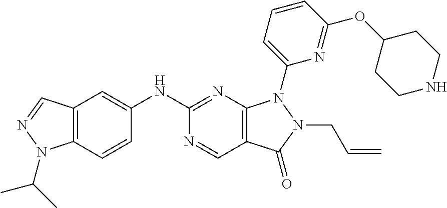 [SMILES: C=CCn1c(=O)c2cnc(Nc3ccc4c(cnn4C(C)C)c3)nc2n1-c1cccc(OC2CCNCC2)n1] | 526.2 | 1H NMR (400 MHz, dmso) δ 10.41 (s, 1H), 8.88 (s, 1H), 8.46 (d, J = 25.3 Hz, 2H), 8.34-7.96 (m, 3H), 7.66 (d, J = 9.1 Hz, 1H), 7.60-7.50 (m, 2H), 6.85 (d, J = 8.2 Hz, 1H), 5.79-5.61 (m, 1H), 5.23-5.14 (m, 1H), 5.06 (d, J = 10.1 Hz, 1H), 5.00-4.88 (m, 2H), 4.62 (s, 2H), 3.28-3.21 (m, 2H), 3.17-3.09 (m, 2H), 2.17-2.05 (m, 2H), 1.95- 1.79 (m, 2H), 1.47 (d, J = 6.6 Hz, 6H). |
| 266 | 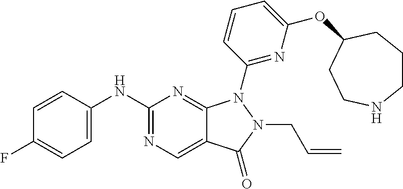 [SMILES: C=CCn1c(=O)c2cnc(Nc3ccc(F)cc3)nc2n1-c1cccc(O[C@H]2CCCNCC2)n1] | 476.5 | 1H-NMR (400 MHz, DMSO-d6): δ 10.31 (br s, 1H), 8.89 (s, 1H), 7.96 (t, J = 8.00 Hz, 1H), 7.77-7.73 (m, 2H), 7.43-7.38 (m, 1H), 7.18 (t, J = 9.20 Hz, 2H), 6.77 (dd, J = 4.40, 8.20 Hz, 1H), 5.76-5.67 (m, 1H), 5.06 (d, J = 9.20 Hz, 1H), 4.92 (d, J = 17.20 Hz, 1H), 4.59 (d, J = 5.20 Hz, 1H), 3.80 (t, J = 4.00 Hz, 1H), 2.93-2.90 (m, 2H), 2.67-2.51 (m, 2H), 1.94-1.90 (m, 2H), 1.52-1.46 (m, 2H) |
| 267 | 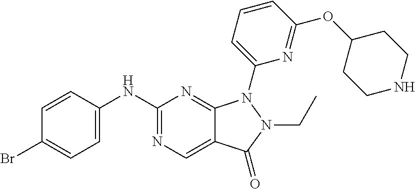 [SMILES: CCn1c(=O)c2cnc(Nc3ccc(Br)cc3)nc2n1-c1cccc(OC2CCNCC2)n1] | 512.0 | 1H NMR: H NMR (400 MHz, dmso) δ 10.4 (s, 1H), 8.90 (s, 1H), 8.5-8.3 (m, 1H), 8.09 (s, 1H), 7.74 (d, J = 8.9 Hz, 2H), 7.51 (d, J = 9.0 Hz, 3H), 6.87 (d, J = 8.2 Hz, 1H), 5.15 (s, 1H), 3.99 (d, J = 6.8 Hz, 2H), 3.17 (d, J = 37.4 Hz, 4H), 2.08 (s, 2H), 1.87 (s, 2H), 1.01 (t, J = 7.0 Hz, 3H). |
| 268 | 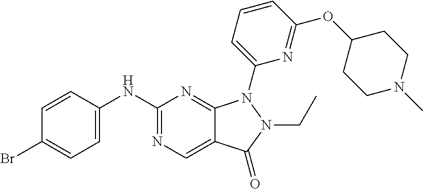 [SMILES: CCn1c(=O)c2cnc(Nc3ccc(Br)cc3)nc2n1-c1cccc(OC2CCN(C)CC2)n1] | 524.0 | 1H NMR: 1H NMR (400 MHz, dmso) δ 10.42 (s, 1H), 9.52 (d, J = 29.9 Hz, 1H), 8.90 (s, 1H), 8.09 (q, J = 8.1 Hz, 1H), 7.74 (d, J = 8.7 Hz, 2H), 7.52 (dd, J = 8.1, 5.7 Hz, 3H), 6.86 (t, J = 7.7 Hz, 1H), 5.25-5.00 (m, 1H), 4.06-3.94 (m, 2H), 3.34 (d, J = 12.1 Hz, 1H), 3.16 (q, J = 10.1 Hz, 2H), 2.80 (dd, J = 13.6, 4.5 Hz, 3H), 2.25 (d, J = 11.6 Hz, 1H), 2.17-1.94 (m, 2H), 1.78 (q, J = 9.8 Hz, 1H), 1.01 (q, J = 6.9 Hz, 3H). |
| 269 | 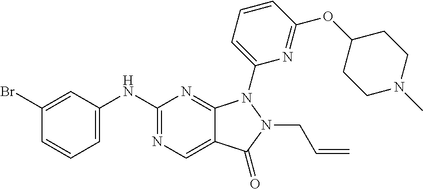 [SMILES: C=CCn1c(=O)c2cnc(Nc3cccc(Br)c3)nc2n1-c1cccc(OC2CCN(C)CC2)n1] | 536.0 | 1H NMR: 1H NMR (400 MHz, dmso) δ 10.52 (s, 1H), 9.35 (s, 1H), 8.94 (s, 1H), 8.29 (s, 1H), 8.11-8.01 (m, 1H), 7.52 (dd, J = 27.7, 7.5 Hz, 2H), 7.31- 7.19 (m, 2H), 6.86 (t, J = 7.8 Hz, 1H), 5.71 (ddd, J = 16.3, 10.4, 5.5 Hz, 1H), 5.30-5.01 (m, 2H), 4.93 (d, J = 17.3 Hz, 1H), 4.62 (s, 2H), 3.47 (s, 1H), 3.18 (s, 2H), 2.86-2.79 (m, 3H), 2.27-2.07 (m, 2H), 2.03 (d, J = 12.4 Hz, 1H), 1.78 (d, J = 13.6 Hz, 1H). |
| 270 | 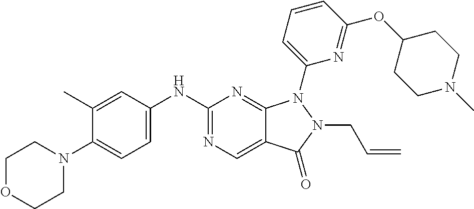 [SMILES: C=CCn1c(=O)c2cnc(Nc3ccc(N4CCOCC4)c(C)c3)nc2n1-c1cccc(OC2CCN(C)CC2)n1] | 557.3 | 1H-NMR (400 MHz, DMSO-d6): δ 10.18 (s, 1H), 8.85 (s, 1H), 7.94 (t, 1H), 7.66 (s, 1H), 7.45 (t, 2H), 7.00 (d, 1H), 6.79 (d, 1H), 5.73-5.68 (m, 1H), 5.07- 4.91 (m, 3H), 4.61 (d, 2H), 3.73 (t, 4H), 2.80 (t, 4H), 2.71 (m, 2H), 2.33 (m, 2H), 2.25 (s, 6H), 1.96 (m, 2H), 1.72 (m, 2H). At 80 °C the peak at δ 2.25 resolves to two singlets: 2.27 (s, 3H) and 2.29 (s, 3H). |
| 271 | 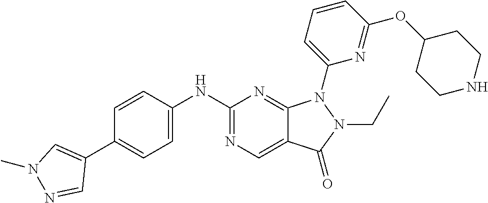 [SMILES: CCn1c(=O)c2cnc(Nc3ccc(-c4cnn(C)c4)cc3)nc2n1-c1cccc(OC2CCNCC2)n1] | 512.0 | 1H NMR: 1H NMR (400 MHz, dmso) δ 8.87 (s, 1H), 8.5-8.3 (m, 2H), 8.07 (s, 2H), 7.82 (s, 1H), 7.73 (d, J = 8.5 Hz, 2H), 7.53 (dd, J = 18.2, 8.1 Hz, 3H), 6.88 (d, J = 8.1 Hz, 1H), 5.16 (s, 1H), 4.00 (s, 2H), 3.86 (s, 3H), 3.18 (d, J = 35.4 Hz, 4H), 2.09 (s, 2H), 1.88 (s, 2H), 1.01 (t, J = 7.0 Hz, 3H). |
| 272 | 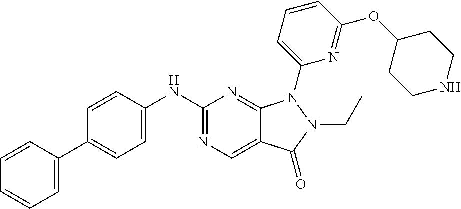 [SMILES: CCn1c(=O)c2cnc(Nc3ccc(-c4ccccc4)cc3)nc2n1-c1cccc(OC2CCNCC2)n1] | 508.0 | 1H NMR: 1H NMR (400 MHz, dmso) δ 8.90 (s, 1H), 8.5-8.0 (m, 3H), 7.86 (d, J = 8.3 Hz, 2H), 7.70-7.56 (m, 6H), 7.45 (d, J = 7.6 Hz, 2H), 7.34 (s, 1H), 6.88 (d, J = 8.1 Hz, 1H), 4.01 (s, 2H), 3.13 (s, 4H), 2.08 (s, 2H), 1.88 (s, 2H), 1.02 (t, J = 7.0 Hz, 3H). |
| 273 | 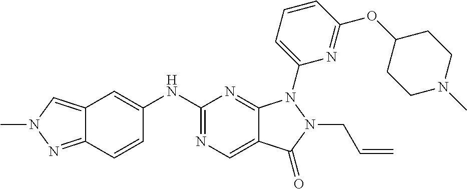 | 512.53 | 1H-NMR (400 MHz, DMSO-d6): δ 10.31 (s, 1H), 8.88 (s, 1H), 8.30 (s, 1H), 8.24 (br s, 1H), 8.01 (t, J = 19.20 Hz, 1H), 7.53 (dd, J = 7.60, 16.00 Hz, 2H), 7.43 (dd, J = 2.00, 9.20 Hz, 1H), 6.79 (d, J = 8.00 Hz, 1H), 5.76-5.69 (m, 1H), 5.06 (dd, J = 1.20, 10.40 Hz, 1H), 4.95 (dd, J = 4.00, 9.00 Hz, 1H), 4.92-4.92 (m, 1H), 4.64-4.63 (m, 2H), 4.16 (s, 3H), 2.68-2.60 (m, 2H), 2.17-1.96 (m, 5H), 1.96-1.93 (m, 2H), 1.70-1.66 (m, 2H). |
| 274 | 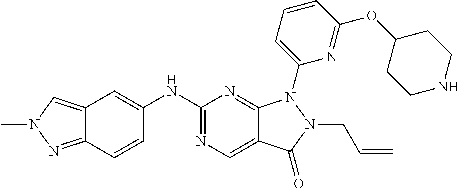 [SMILES: C=CCn1c(=O)c2cnc(Nc3ccc4nn(C)cc4c3)nc2n1-c1cccc(OC2CCNCC2)n1] | 498.51 | 1H-NMR (400 MHz, DMSO-d6,): δ 10.32 (s, 1H), 8.89 (s, 1H), 8.31 (d, J = 10.00 Hz, 1H), 8.24 (br s, 1H), 8.03 (s, 1H), 7.54 (dd, J = 8.00, 11.40 Hz, 2H), 7.43 (dd, J = 2.00, 9.20 Hz, 1H), 6.81 (d, J = 8.00 Hz, 1H), 5.72-5.67 (m, 1H), 5.08 (dd, J = 9.20, 8.00 Hz, 1H), 4.98 (dd, J = 16.00, 25.80 Hz, 1H), 4.93-4.92 (m, 1H), 4.93-4.92 (m, 2H), 4.16 (s, 3H), 3.08-3.04 (m, 2H), 2.79-2.75 (m, 5H), 2.01-1.98 (m, 2H), 1.68-1.63 (m, 2H). |
| 275 | 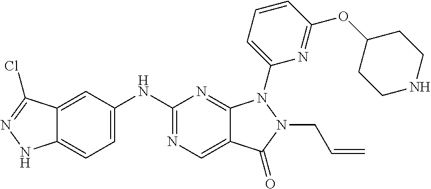 [SMILES: C=CCn1c(=O)c2cnc(Nc3ccc4[nH]nc(Cl)c4c3)nc2n1-c1cccc(OC2CCNCC2)n1] | 518.3 | 1H-NMR (400 MHz, DMSO-d6): δ 13.25 (s, 1H), 8.92 (s, 1H), 8.27 (s, 2H), 7.97 (t, J = 8.00 Hz, 1H), 7.62-7.60 (m, 1H), 7.54-7.49 (m, 2H), 6.84 (d, J = 8.00 Hz, 1H), 5.77-5.67 (m, 1H), 5.06 (d, J = 9.20 Hz, 1H), 4.94 (d, J = 17.20 Hz, 1H), 4.61 (d, J = 5.60 Hz, 2H), 3.07- 3.04 (m, 2H), 2.78 (t, J = 9.60 Hz, 2H), 2.01-1.98 (m, 2H), 1.69-1.64 (m, 2H). |
| 276 | 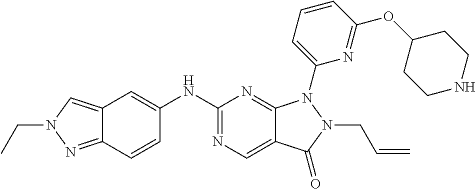 [SMILES: C=CCn1c(=O)c2cnc(Nc3ccc4nn(CC)cc4c3)nc2n1-c1cccc(OC2CCNCC2)n1] | 512.53 | 1H-NMR (400 MHz, DMSO-d6): δ 9.98 (s, 1H), 8.84 (s, 1H), 8.25 (s, 1H), 8.13 (d, J = 1.20 Hz, 1H), 7.95 (t, J = 7.60 Hz, 1H), 7.55 (d, J = 9.20 Hz, 1H), 7.49- 7.44 (m, 2H), 6.77 (d, J = 8.00 Hz, 1H), 5.76-5.71 (m, 1H), 5.08 (dd, J = 1.20, 10.20 Hz, 1H), 5.02-4.97 (m, 2H), 4.62 (d, J = 6.00 Hz, 2H), 4.45 (q, J = 7.20 Hz, 2H), 3.08-2.94 (m, 2H), 2.62-2.55 (m, 2H), 1.95-1.57 (m, 2H), 1.57-1.51 (m, 5H). |
| 277 | 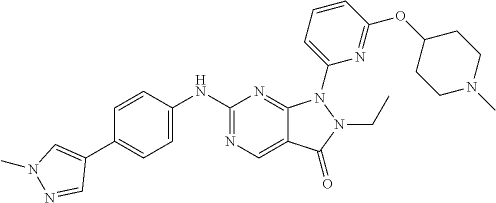 | 526.0 | 1H NMR: 1H NMR (400 MHz, dmso) δ 9.33 (s, 1H), 8.87 (s, 1H), 8.08 (s, 2H), 7.82 (s, 1H), 7.73 (d, J = 8.4 Hz, 2H), 7.60-7.47 (m, 3H), 6.93-6.79 (m, 1H), 5.16 (d, J = 65.7 Hz, 1H), 4.00 (s, 2H), 3.86 (s, 3H), 3.33 (d, J = 10.6 Hz, 1H), 3.16 (d, J = 12.5 Hz, 2H), 2.80 (dd, J = 11.0, 4.7 Hz, 3H), 2.24 (s, 1H), 2.12 (d, J = 14.5 Hz, 1H), 2.07 (s, 1H), 2.01 (d, J = 14.3 Hz, 1H), 1.78 (d, J = 13.8 Hz, 1H), 1.01 (q, J = 7.0 Hz, 3H). |
| 278 | 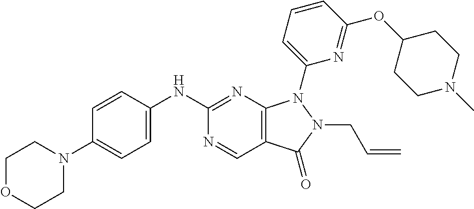 [SMILES: C=CCn1c(=O)c2cnc(Nc3ccc(N4CCOCC4)cc3)nc2n1-c1cccc(OC2CCN(C)CC2)n1] | 543.0 | 1H NMR (400 MHz, cd3od) δ 8.79 (s, 1H), 7.87 (t, J = 7.9 Hz, 1H), 7.55 (d, J = 8.6 Hz, 2H), 7.41 (d, J = 7.6 Hz, 1H), 6.94 (d, J = 9.0 Hz, 2H), 6.76 (d, J = 8.2 Hz, 1H), 5.75 (ddt, J = 16.4, 10.6, 5.9 Hz, 1H), 5.09 (d, J = 10.3 Hz, 2H), 4.98 (d, J = 17.2 Hz, 1H), 4.70 (d, J = 5.8 Hz, 2H), 3.89-3.81 (m, 4H), 3.16-3.08 (m, 4H), 2.72 (s, 2H), 2.36 (s, 2H), 2.30 (s, 3H), 2.04 (s, 2H), 1.84 (s, 2H). |
| 279 | 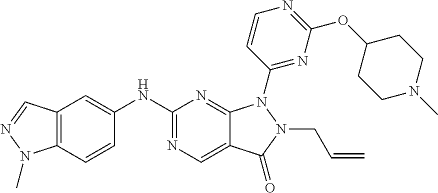 [SMILES: C=CCn1c(=O)c2cnc(Nc3ccc4c(cnn4C)c3)nc2n1-c1ccnc(OC2CCN(C)CC2)n1] | 513.49 | 1H-NMR (400 MHz, DMSO-d6): δ 10.57 (br s, 1H), 8.93 (s, 1H), 8.66 (d, J = 5.60 Hz, 1H), 8.14-8.09 (m, 2H), 7.76-7.66 (m, 3H), 5.73-5.65 (m, 1H), 5.08-4.97 (m, 3H), 4.81-4.75 (m, 2H), 4.06 (s, 3H), 2.68-2.63 (m, 2H), 2.19- 2.16 (m, 5H), 2.01-1.99 (m, 2H), 1.77- 1.73 (m, 2H) |
| 280 | 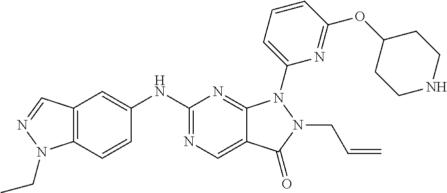 [SMILES: C=CCn1c(=O)c2cnc(Nc3ccc4c(cnn4CC)c3)nc2n1-c1cccc(OC2CCNCC2)n1] | 512.59 | 1H-NMR (400 MHz, DMSO-d6,): δ 10.39 (s, 1H), 8.89 (s, 1H), 8.31-8.25 (m, 2H), 8.03-7.99 (m, 2H), 7.65-7.58 (m, 2H), 7.51 (d, J = 7.60 Hz, 1H), 6.82 (d, J = 8.40 Hz, 1H), 5.76-5.67 (m, 1H), 5.09-5.05 (m, 2H), 4.94 (dd, J = 0.80, 17.20 Hz, 1H), 4.63 (d, J = 4.00 Hz, 2H), 4.43 (q, J = 7.20 Hz, 2H), 3.09- 3.03 (m, 2H), 2.82-2.76 (m, 2H), 2.01- 1.68 (m, 2H), 1.68-1.64 (m, 2H), 1.40 (t, J = 7.20 Hz, 3H). |
| 281 | 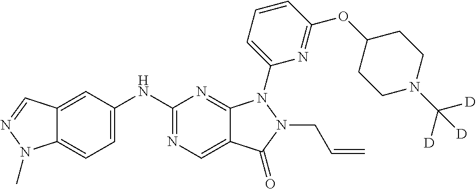 [SMILES: [2H]C([2H])([2H])N1CCC(Oc2cccc(-n3c4nc(Nc5ccc6c(cnn6C)c5)ncc4c(=O)n3CC=C)n2)CC1] | 515.6 | 1H-NMR (400 MHz, DMSO-d6): 1H- NMR (400 MHz, DMSO-d6): δ 10.36 (s, 1H), 8.88 (s, 1H), 8.24 (s, 1H), 8.01- 7.97 (m, 2H), 7.64-7.58 (m, 2H), 7.49 (d, J = 7.60 Hz, 1H), 6.79 (d, J = 8.00 Hz, 1H), 5.77-5.67 (m, 1H), 5.06 (dd, J = 1.20, 10.20 Hz, 1H), 4.97-4.92 (m, 2H), 4.63-4.62 (m, 2H), 4.04 (s, 3H), 2.60 (t, J = 6.00 Hz, 2H), 2.16 (t, J = 9.20 Hz, 2H), 1.95-1.92 (m, 2H), 1.71- 1.63 (m, 2H). |
| 282 | 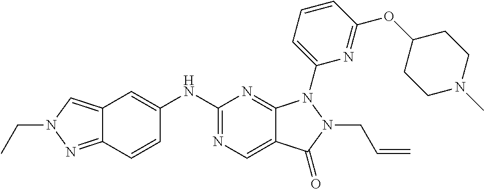 [SMILES: C=CCn1c(=O)c2cnc(Nc3ccc4nn(CC)cc4c3)nc2n1-c1cccc(OC2CCN(C)CC2)n1] | 526.5 | 1H-NMR (400 MHz, DMSO-d6): δ 10.30 (br s, 1H), 8.88 (s, 1H), 8.33 (s, 1H), 8.23-8.17 (m, 1H), 8.03 (s, 1H), 7.54 (dd, J = 7.60, 20.40 Hz, 2H), 7.43 (dd, J = 2.00, 9.20 Hz, 1H), 6.80 (d, J = 8.40 Hz, 1H), 5.76-5.69 (m, 1H), 5.06 (dd, J = 1.20, 10.40 Hz, 1H), 4.97-4.92 (m, 2H), 4.63 (s, 2H), 4.44 (q, J = 7.20 Hz, 2H), 2.66 (s, 2H), 2.35-2.34 (m, 5H), 1.97-1.94 (m, 2H), 1.73-1.66 (m, 2H), 1.52 (t, J = 7.20 Hz, 3H). |
| 284 | 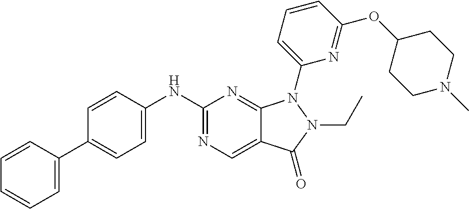 [SMILES: CCn1c(=O)c2cnc(Nc3ccc(-c4ccccc4)cc3)nc2n1-c1cccc(OC2CCN(C)CC2)n1] | 522.0 | 1H NMR: 1H NMR (400 MHz, dmso) δ 10.44 (s, 1H), 9.34 (s, 1H), 8.90 (s, 1H), 8.12 (d, J = 7.9 Hz, 1H), 7.86 (d, J = 8.0 Hz, 2H), 7.67 (t, J = 6.9 Hz, 3H), 7.59 (d, J = 7.7 Hz, 1H), 7.46 (t, J = 7.6 Hz, 2H), 7.34 (t, J = 7.3 Hz, 1H), 6.86 (t, J = 7.5 Hz, 1H), 5.24 (s, 1H), 5.08 (s, 1H), 4.05-3.96 (m, 2H), 3.32 (s, 1H), 3.21-3.12 (m, 2H), 2.80 (dd, J = 10.3, 4.6 Hz, 3H), 2.26 (d, J = 13.0 Hz, 1H), 2.16-2.05 (m, 1H), 2.02 (d, J = 13.0 Hz, 1H), 1.78 (d, J = 12.8 Hz, 1H), 1.02 (q, J = 6.8 Hz, 3H). |
| 285 | 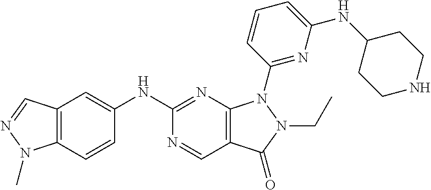 [SMILES: CCn1c(=O)c2cnc(Nc3ccc4c(cnn4C)c3)nc2n1-c1cccc(NC2CCNCC2)n1] | 485.3 | 1H NMR (400 MHz, dmso) δ 10.29 (s, 1H), 8.83 (s, 1H), 8.47 (s, 1H), 8.39- 8.21 (m, 2H), 7.98 (s, 1H), 7.74 (s, 1H), 7.65-7.54 (m, 2H), 7.04 (d, J = 7.1 Hz, 2H), 6.52 (d, J = 8.3 Hz, 1H), 4.05- 3.95 (m, 5H), 3.88 (s, 1H), 3.27 (d, J = 12.0 Hz, 2H), 3.01 (q, J = 9.9 Hz, 2H), 2.01 (d, J = 11.5 Hz, 2H), 1.58 (q, J = 10.4 Hz, 2H), 1.01 (t, J = 6.9 Hz, 3H). |
| 286 | 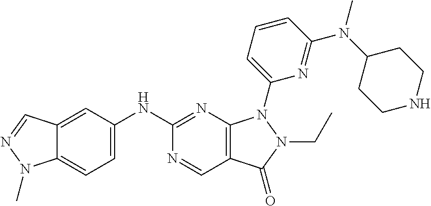 [SMILES: CCn1c(=O)c2cnc(Nc3ccc4c(cnn4C)c3)nc2n1-c1cccc(N(C)C2CCNCC2)n1] | 499.4 | 1H NMR (400 MHz, Methanol-d4) δ 8.76 (s, 1H), 8.23 (s, 1H), 7.87 (s, 1H), 7.79 (t, J = 7.9 Hz, 1H), 7.51 (d, J = 9.0 Hz, 1H), 7.43 (d, J = 9.0 Hz, 1H), 7.10 (d, J = 7.5 Hz, 1H), 6.70 (d, J = 8.4 Hz, 1H), 4.78 (t, J = 11.7 Hz, 1H), 4.14 (q, J = 6.8 Hz, 2H), 4.02 (s, 3H), 3.48 (d, J = 12.2 Hz, 2H), 3.11 (t, J = 12.1 Hz, 2H), 2.94 (s, 3H), 2.02 (q, J = 12.4 Hz, 2H), 1.88 (d, J = 12.5 Hz, 2H), 1.11 (t, J = 7.0 Hz, 3H). Two exchangeable proton signals not listed. |
| 287 | 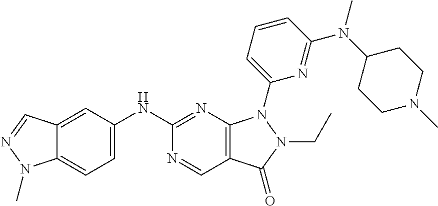 [SMILES: CCn1c(=O)c2cnc(Nc3ccc4c(cnn4C)c3)nc2n1-c1cccc(N(C)C2CCN(C)CC2)n1] | 513.0 | 1H NMR (400 MHz, Methanol-d4) δ 8.80 (s, 1H), 8.28 (s, 1H), 7.91 (s, 1H), 7.76 (t, J = 8.1 Hz, 1H), 7.57 (d, J = 8.9 Hz, 1H), 7.49 (d, J = 8.5 Hz, 1H), 7.08 (d, J = 7.4 Hz, 1H), 6.64 (d, J = 8.3 Hz, 1H), 4.49 (s, 1H), 4.17 (q, J = 7.0 Hz, 2H), 4.06 (s, 3H), 2.96 (d, J = 12.0 Hz, 2H), 2.92 (s, 3H), 2.30 (s, 3H), 2.15 (t, J = 12.0 Hz, 2H), 1.92-1.78 (m, 2H), 1.64 (d, J = 11.1 Hz, 2H), 1.12 (t, J = 7.1 Hz, 3H). One exchangeable proton signal not listed. |
| 288 | 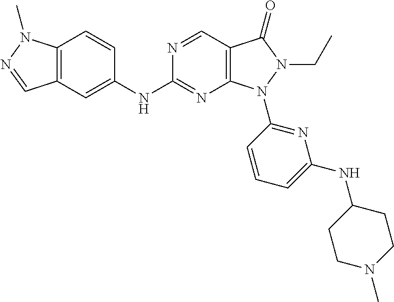 [SMILES: CCn1c(=O)c2cnc(Nc3ccc4c(cnn4C)c3)nc2n1-c1cccc(NC2CCN(C)CC2)n1] | 499.0 | 1H NMR (400 MHz, dmso) δ 10.29 (s, 1H), 9.19 (s, 1H), 8.83 (s, 1H), 8.34 (s, 1H), 7.98 (s, 1H), 7.74 (s, 1H), 7.66- 7.52 (m, 2H), 7.08-6.99 (m, 2H), 6.50 (d, J = 8.3 Hz, 1H), 4.03-3.97 (m, 5H), 3.83 (s, 1H), 3.42 (d, J = 11.7 Hz, 2H), 3.08 (q, J = 11.4 Hz, 2H), 2.76 (d, J = 4.1 Hz, 3H), 2.09 (d, J = 14.0 Hz, 2H), 1.57 (q, J = 12.1 Hz, 2H), 1.01 (t, J = 6.6 Hz, 3H). |
| 289 | 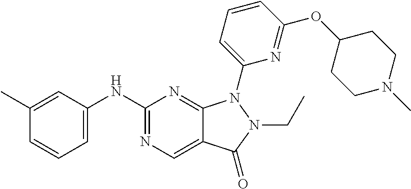 | 460.0 | 1H NMR (400 MHz, DMSO-d6) δ 10.25 (s, 1H), 9.62-9.38 (m, 1H), 8.87 (s, 1H), 8.07-7.95 (m, 1H), 7.70 (s, 1H), 7.57 (d, J = 7.6 Hz, 1H), 7.44 (d, J = 8.0 Hz, 1H), 7.20 (t, J = 7.8 Hz, 1H), 6.91-6.76 (m, 2H), 5.32-4.96 (m, 1H), 4.07-3.94 (m, 2H), 3.48 (d, J = 11.5 Hz, 1H), 3.33 (d, J = 11.4 Hz, 1H), 3.16 (q, J = 11.6, 10.2 Hz, 2H), 2.87- 2.72 (m, 3H), 2.35-2.18 (m, 4H), 2.16- 2.06 (m, 1H), 2.07-1.95 (m, 1H), 1.86-1.70 (m, 1H), 1.06-0.94 (m, 3H). |
| 290 | 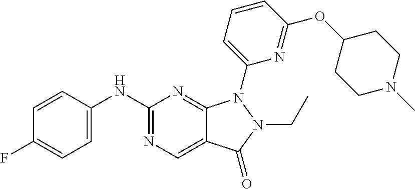 [SMILES: CCn1c(=O)c2cnc(Nc3ccc(F)cc3)nc2n1-c1cccc(OC2CCN(C)CC2)n1] | 464.0 | 1H NMR (400 MHz, DMSO-d6) δ 10.50-10.18 (m, 1H), 9.64-9.40 (m, 1H), 8.87 (s, 1H), 8.05 (q, J = 8.6, 8.1 Hz, 1H), 7.78-7.68 (m, 2H), 7.53 (d, J = 7.7 Hz, 1H), 7.18 (t, J = 8.8 Hz, 2H), 6.85 (t, J = 7.4 Hz, 1H), 5.28-4.99 (m, 1H), 3.99 (d, J = 6.2 Hz, 2H), 3.48 (d, J = 12.5 Hz, 1H), 3.34 (d, J = 11.5 Hz, 1H), 3.16 (q, J = 12.0, 11.5 Hz, 2H), 2.80 (dd, J = 13.2, 4.3 Hz, 3H), 2.30- 2.19 (m, 1H), 2.16-2.06 (m, 1H), 2.06- 1.93 (m, 1H), 1.86-1.70 (m, 1H), |
| | | | 1.00 (q, J = 6.9 Hz, 3H). |
| 291 | 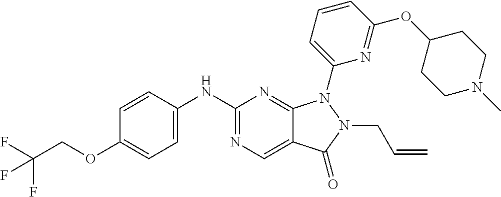 [SMILES: C=CCn1c(=O)c2cnc(Nc3ccc(OCC(F)(F)F)cc3)nc2n1-c1cccc(OC2CCN(C)CC2)n1] | 556.0 | 1H NMR (400 MHz, dmso) δ 10.25 (s, 1H), 8.86 (s, 1H), 7.96 (t, J = 7.7 Hz, 1H), 7.67 (d, J = 8.5 Hz, 2H), 7.42 (d, J = 7.7 Hz, 1H), 7.04 (d, J = 9.0 Hz, 2H), 6.77 (d, J = 8.1 Hz, 1H), 5.70 (ddt, J = 16.2, 10.7, 5.8 Hz, 1H), 5.05 (d, J = 10.3 Hz, 1H), 5.00-4.87 (m, 2H), 4.73 (q, J = 8.9 Hz, 3H), 4.61 (d, J = 4.2 Hz, 2H), 2.67-2.54 (m, 2H), 2.20 (s, 3H), 1.93 (d, J = 9.8 Hz, 2H), 1.67 (td, J = 12.9, 6.1 Hz, 2H). |
| 292 | 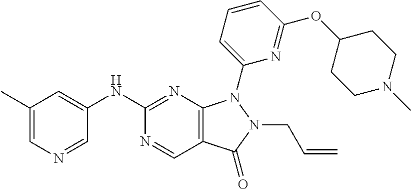 | 473.0 | 1H NMR (400 MHz, dmso) δ 10.42 (s, 1H), 8.93 (s, 1H), 8.60 (s, 1H), 8.10 (d, J = 15.7 Hz, 2H), 7.93 (t, J = 7.9 Hz, 1H), 7.42 (d, J = 7.6 Hz, 1H), 6.81 (d, J = 8.2 Hz, 1H), 5.80-5.61 (m, 1H), 5.05 (d, J = 10.3 Hz, 1H), 4.99-4.87 (m, 2H), 4.60 (d, J = 5.4 Hz, 2H), 2.64-2.56 (m, 2H), 2.29 (s, 3H), 2.20-2.10 (m, 5H), 1.99-1.88 (m, 2H), 1.73-1.60 (m, 2H). |
| 293 | 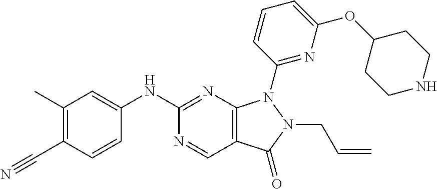 [SMILES: C=CCn1c(=O)c2cnc(Nc3ccc(C#N)c(C)c3)nc2n1-c1cccc(OC2CCNCC2)n1] | 483.0 | 1H NMR (400 MHz, DMSO-d6) δ 10.68 (s, 1H), 8.97 (s, 1H), 8.05-7.91 (m, 2H), 7.72-7.62 (m, 2H), 7.43 (d, J = 7.6 Hz, 1H), 6.82 (d, J = 8.2 Hz, 1H), 5.80-5.60 (m, 1H), 5.05 (d, J = 10.4 Hz, 1H), 5.02-4.84 (m, 2H), 4.62 (d, J = 5.4 Hz, 2H), 2.99-2.87 (m, 2H), 2.58- 2.53 (m, 2H), 2.44 (s, 3H), 1.95-1.84 (m, 2H), 1.56-1.43 (m, 2H). One exchangeable proton signal not listed. |
| 294 | 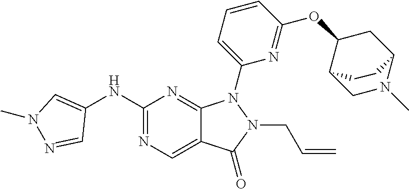 | 488.0 | 1H NMR (500 MHz, DMSO) δ 10.18 (d, J = 138.4 Hz, 1H), 8.83 (d, J = 38.0 Hz, 1H), 8.00 (t, J = 7.9 Hz, 1H), 7.93- 7.82 (m, 1H), 7.51 (d, J = 13.1 Hz, 1H), 7.42 (d, J = 7.6 Hz, 1H), 6.83 (d, J = 8.2 Hz, 1H), 5.69 (ddt, J = 16.2, 10.6, 5.8 Hz, 1H), 5.04 (d, J = 1.3 Hz, 1H), 4.98 (d, J = 9.0 Hz, 1H), 4.91 (dd, J = 17.5, 9.2 Hz, 1H), 4.64-4.52 (m, 2H), 3.80 (s, 3H), 2.94 (d, J = 9.6 Hz, 1H), 2.54- 2.51 (m, 2H), 2.27 (s, 3H), 2.07-1.98 (m, 1H), 1.92-1.79 (m, 3H), 1.64 (t, |
| | | | J = 12.1 Hz, 1H), 1.52 (t, J = 10.3 Hz, |
| | | | 1H), 1.36 (t, J = 10.4 Hz, 1H). Mixture |
| | | | of two conformers 1:3. |
| 295 | 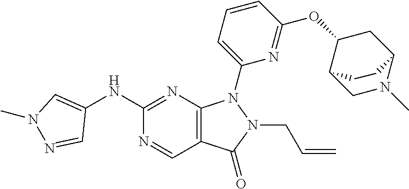 | 488.3 | 1H NMR (500 MHz, DMSO) δ 10.18 (d, J = 140.2 Hz, 1H), 8.83 (d, J = 38.1 Hz, 1H), 8.00 (t, J = 7.9 Hz, 1H), 7.94- 7.80 (m, 1H), 7.51 (d, J = 13.4 Hz, 1H), 7.42 (d, J = 7.6 Hz, 1H), 6.87-6.75 (m, 1H), 5.69 (ddt, J = 16.2, 10.8, 5.8 Hz, 1H), 5.08 (d, J = 8.9 Hz, 1H), 5.06- 5.02 (m, 1H), 4.90 (d, J = 17.1 Hz, 1H), 4.64-4.50 (m, 2H), 3.80 (s, 3H), 2.65 (d, J = 10.3 Hz, 1H), 2.58 (dd, J = 10.1, 2.6 Hz, 1H), 2.48-2.41 (m, 2H), 2.21 (s, 3H), 1.94-1.90 (m, 1H), 1.89-1.81 |
| | | | (m, 2H), 1.46 (dd, J = 19.2, 11.4 Hz, |
| | | | 2H), 1.42-1.34 (m, 1H). Mixture of |
| | | | two conformers 1:3. |
| 296 | 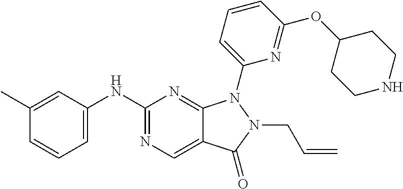 [SMILES: C=CCn1c(=O)c2cnc(Nc3cccc(C)c3)nc2n1-c1cccc(OC2CCNCC2)n1] | 456.40 | 1H-NMR (400 MHz, DMSO-d6): δ 10.25 (bs, 1H), 8.89 (s, 1H), 8.34 (s, 1H), 7.96-7.92 (t, J = 16 Hz 1H), 7.70 (bs, 1H), 7.47-7.43 (m, 2H), 7.21-7.17 (t, J = 15.6 Hz, 1H), 6.87-6.80 (m, 2H), 5.74-5.68 (m, 1H), 5.08-5.04 (m 3H), 4.62-4.60 (d, J = 6 Hz, 2H), 3.06-3.03 (m, 2H), 2.79-2.74(m, 2H), 2.29 (s, 3H), 2.00-1.90, 1.69-1.60 (m, 2H). |
| 297 | 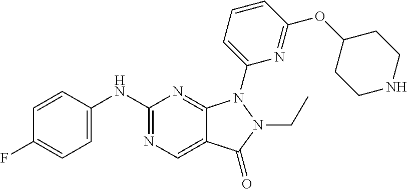 [SMILES: CCn1c(=O)c2cnc(Nc3ccc(F)cc3)nc2n1-c1cccc(OC2CCNCC2)n1] | 450.0 | 1H NMR (400 MHz, DMSO-d6) δ 10.32 (s, 1H), 8.86 (s, 1H), 8.75-8.52 (m, 2H), 8.04 (t, J = 7.8 Hz, 1H), 7.79- 7.67 (m, 2H), 7.51 (d, J = 7.7 Hz, 1H), 7.24-7.12 (m, 2H), 6.86 (d, J = 8.1 Hz, 1H), 5.22-5.10 (m, 1H), 3.99 (q, J = 6.4 Hz, 2H), 3.30-3.18 (m, 2H), 3.17- 3.05 (m, 2H), 2.17-2.04 (m, 2H), 1.96- 1.76 (m, 2H), 1.00 (t, J = 7.0 Hz, 3H). |
| 298 | 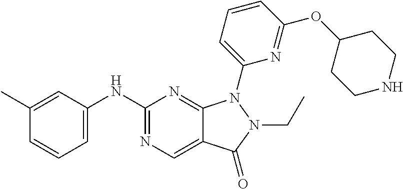 [SMILES: CCn1c(=O)c2cnc(Nc3cccc(C)c3)nc2n1-c1cccc(OC2CCNCC2)n1] | 446.0 | 1H NMR (400 MHz, DMSO-d6) δ 10.24 (s, 1H), 8.86 (s, 1H), 8.50 (d, J = 18.1 Hz, 2H), 8.01 (t, J = 7.9 Hz, 1H), 7.69 (s, 1H), 7.55 (d, J = 7.7 Hz, 1H), 7.44 (d, J = 7.9 Hz, 1H), 7.19 (t, J = 7.8 Hz, 1H), 6.93-6.81 (m, 2H), 5.15 (d, J = 3.7 Hz, 1H), 3.99 (q, J = 6.6 Hz, 2H), 3.31-3.18 (m, 3H), 3.17-3.04 (m, 2H), 2.29 (s, 3H), 2.17-2.02 (m, 2H), 1.97-1.77 (m, 2H), 1.00 (t, J = 7.0 Hz, 3H). |
| 299 | 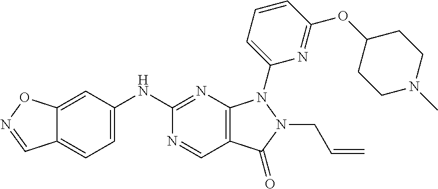 [SMILES: C=CCn1c(=O)c2cnc(Nc3ccc4cnoc4c3)nc2n1-c1cccc(OC2CCN(C)CC2)n1] | 499.0 | 1H NMR (400 MHz, dmso) δ 10.55 (s, 1H), 8.96 (s, 1H), 7.95 (t, J = 7.9 Hz, 1H), 7.51 (d, J = 8.6 Hz, 1H), 7.46 (d, J = 7.6 Hz, 2H), 7.40 (d, J = 8.4 Hz, 1H), 6.80 (d, J = 8.2 Hz, 1H), 5.81-5.64 (m, 1H), 5.06 (d, J = 10.2 Hz, 1H), 5.00- 4.87 (m, 2H), 4.61 (d, J = 5.4 Hz, 2H), 2.63-2.56 (m, 2H), 2.23-2.07 (m, 6H), 1.98-1.87 (m, 2H), 1.66 (q, J = 8.7 Hz, 2H). |
| 300 | 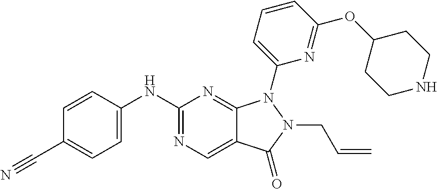 [SMILES: C=CCn1c(=O)c2cnc(Nc3ccc(C#N)cc3)nc2n1-c1cccc(OC2CCNCC2)n1] | 469.0 | 1H NMR 1H NMR (400 MHz, dmso) δ 10.75 (s, 1H), 9.00 (s, 1H), 8.46 (s, 2H), 8.08 (t, J = 7.9 Hz, 1H), 7.97 (d, J = 8.8 Hz, 2H), 7.79 (d, J = 8.8 Hz, 2H), 7.47 (d, J = 7.7 Hz, 1H), 6.88 (d, J = 8.2 Hz, 1H), 5.71 (ddd, J = 16.3, 10.7, 5.4 Hz, 1H), 5.18 (s, 1H), 5.06 (d, J = 9.4 Hz, 1H), 4.94 (d, J = 17.2 Hz, 1H), 4.62 (m, 2H), 3.18 (m, 4H), 2.10 (m, 2H), 1.88 (m, 2H). |
| 301 | 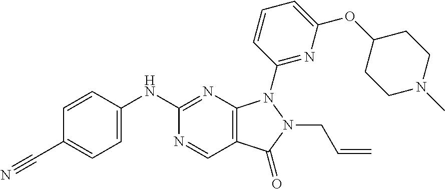 [SMILES: C=CCn1c(=O)c2cnc(Nc3ccc(C#N)cc3)nc2n1-c1cccc(OC2CCN(C)CC2)n1] | 483 | 1H NMR: (400 MHz, dmso) δ 10.74 (s, 1H), 8.98 (s, 1H), 8.15-7.91 (m, 3H), 7.78 (d, J = 8.7 Hz, 2H), 7.42 (d, J = 7.6 Hz, 1H), 6.81 (d, J = 8.1 Hz, 1H), 5.80- 5.61 (m, 1H), 5.06 (d, J = 10.4 Hz, 1H), 4.99-4.83 (m, 2H), 4.62 (d, J = 5.6 Hz, 2H), 2.6-2.4 (m, 4H), 2.16 (s, 3H), 1.92 (s, 2H), 1.67 (d, J = 9.0 Hz, 2H). |
| 302 | 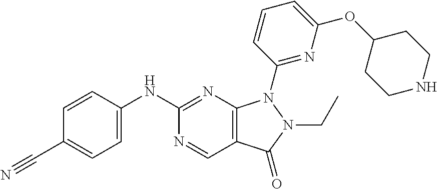 [SMILES: CCn1c(=O)c2cnc(Nc3ccc(C#N)cc3)nc2n1-c1cccc(OC2CCNCC2)n1] | 457.0 | 1H NMR (400 MHz, DMSO-d6) δ 10.73 (s, 1H), 8.97 (s, 1H), 8.62-8.39 (m, 2H), 8.11 (t, J = 8.0 Hz, 1H), 7.97 (d, J = 8.8 Hz, 2H), 7.79 (d, J = 8.8 Hz, 2H), 7.53 (d, J = 7.7 Hz, 1H), 6.90 (d, J = 8.2 Hz, 1H), 5.21-5.09 (m, 1H), 4.01 (q, J = 6.8 Hz, 2H), 3.30-3.18 (m, 2H), 3.17-3.05 (m, 2H), 2.17-2.02 (m, 2H), 1.96-1.74 (m, 2H), 1.02 (t, J = 7.0 Hz, 3H). |
| 303 | 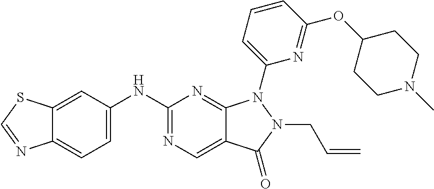 [SMILES: C=CCn1c(=O)c2cnc(Nc3ccc4ncsc4c3)nc2n1-c1cccc(OC2CCN(C)CC2)n1] | 515.0 | 1H NMR (400 MHz, dmso) δ 10.62 (s, 1H), 9.47-9.31 (m, 1H), 9.29 (s, 1H), 8.95 (s, 1H), 8.72 (s, 1H), 8.10-7.94 (m, 2H), 7.74 (d, J = 8.9 Hz, 1H), 7.56 (dd, J = 7.5, 2.7 Hz, 1H), 6.92-6.83 (m, 1H), 5.82-5.63 (m, 1H), 5.30- 5.03 (m, 2H), 4.94 (d, J = 17.2 Hz, 1H), 4.69-4.57 (m, 2H), 3.48 (d, J = 10.9 Hz, 1H), 3.34 (d, J = 11.7 Hz, 1H), 3.22- 3.10 (m, 2H), 2.80 (dd, J = 11.6, 4.6 Hz, 3H), 2.27 (d, J = 12.4 Hz, 1H), 2.16- 1.94 (m, 2H), 1.78 (q, J = 10.5 Hz, 1H). |
| 304 | 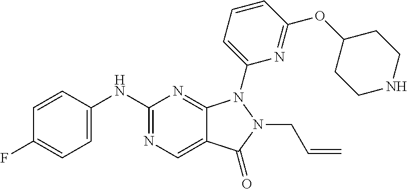 [SMILES: C=CCn1c(=O)c2cnc(Nc3ccc(F)cc3)nc2n1-c1cccc(OC2CCNCC2)n1] | 462.0 | 1H NMR (400 MHz, dmso) δ 10.35 (s, 1H), 8.89 (s, 1H), 8.58 (s, 2H), 8.01 (t, J = 7.9 Hz, 1H), 7.74 (dd, J = 8.9, 4.9 Hz, 2H), 7.46 (d, J = 7.7 Hz, 1H), 7.18 (t, J = 8.9 Hz, 2H), 6.84 (d, J = 8.2 Hz, 1H), 5.70 (ddt, J = 16.3, 10.6, 5.8 Hz, 1H), 5.18 (s, 1H), 5.05 (d, J = 10.3 Hz, 1H), 4.93 (d, J = 16.1 Hz, 1H), 4.61 (d, J = 5.0 Hz, 2H), 3.19 (d, J = 33.8 Hz, 4H), 2.10 (s, 2H), 1.87 (d, J = 9.5 Hz, 2H). |
| 305 | 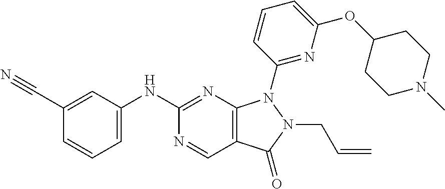 [SMILES: C=CCn1c(=O)c2cnc(Nc3cccc(C#N)c3)nc2n1-c1cccc(OC2CCN(C)CC2)n1] | 483.0 | 1H NMR 1H NMR (400 MHz, cd3od) δ 8.90 (s, 1H), 8.52 (s, 1H), 8.01 (t, J = 7.9 Hz, 1H), 7.72 (d, J = 8.1 Hz, 1H), 7.53-7.41 (m, 2H), 7.38 (d, J = 7.6 Hz, 1H), 6.81 (d, J = 8.2 Hz, 1H), 5.76 (ddd, J = 16.3, 10.4, 5.9 Hz, 1H), 5.10 (d, J = 10.3 Hz, 2H), 4.99 (d, J = 17.1 Hz, 1H), 4.74 (d, J = 5.9 Hz, 3H), 2.73 (s, 2H), 2.39 (s, 2H), 2.31 (s, 3H), 2.04 (s, 2H), 1.84 (d, J = 8.6 Hz, 2H). |
| 306 | 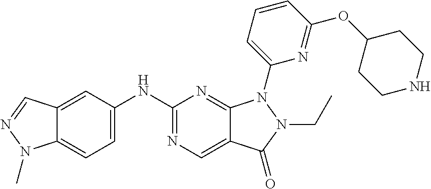 [SMILES: CCn1c(=O)c2cnc(Nc3ccc4c(cnn4C)c3)nc2n1-c1cccc(OC2CCNCC2)n1] | 486.0 | 1H NMR (400 MHz, Chloroform-d) δ 8.85 (s, 1H), 8.14 (s, 1H), 7.93 (s, 1H), 7.75 (t, J = 7.9 Hz, 1H), 7.46-7.40 (m, 2H), 7.37 (d, J = 8.9 Hz, 1H), 6.70 (d, J = 8.1 Hz, 1H), 5.08 (tt, J = 8.6, 3.9 Hz, 1H), 4.21 (q, J = 7.0 Hz, 2H), 4.10 (s, 3H), 3.12 (dt, J = 12.2, 4.2 Hz, 2H), 2.81-2.68 (m, 2H), 2.00 (m, 2H), 1.67 (dtd, J = 12.9, 9.3, 3.8 Hz, 2H), 1.11 (t, J = 7.1 Hz, 3H). |
| 307 | 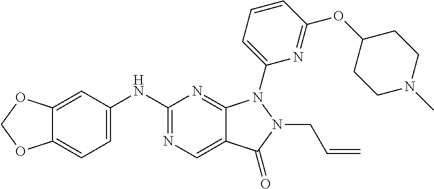 [SMILES: C=CCn1c(=O)c2cnc(Nc3ccc4c(c3)OCO4)nc2n1-c1cccc(OC2CCN(C)CC2)n1] | 502.0 | 1H NMR (500 MHz, DMSO) δ 10.25 (s, 1H), 9.44 (d, J = 30.1 Hz, 1H), 8.87 (s, 1H), 7.95 (dt, J = 11.8, 7.9 Hz, 1H), 7.47 (d, J = 7.6 Hz, 1H), 7.44 (s, 1H), 7.12 (d, J = 8.4 Hz, 1H), 6.89 (d, J = 8.4 Hz, 1H), 6.84 (dd, J = 10.3, 8.1 Hz, 1H), 6.01 (d, J = 1.4 Hz, 2H), 5.77-5.64 (m, 1H), 5.30-5.02 (m, 2H), 4.94 (dt, J = 17.2, 1.4 Hz, 1H), 4.60 (t, J = 6.7 Hz, 2H), 3.50 (d, J = 12.4 Hz, 1H), 3.35 (d, J = 12.2 Hz, 1H), 3.23-3.10 (m, 2H), 2.82 (dd, J = 15.8, 4.7 Hz, 3H), 2.28 (d, |
| | | | J = 13.4 Hz, 1H), 2.13 (d, J = 15.4 Hz, |
| | | | 1H), 2.02 (t, J = 14.2 Hz, 1H), 1.85- |
| | | | 1.74 (m, 1H). |
| 308 | 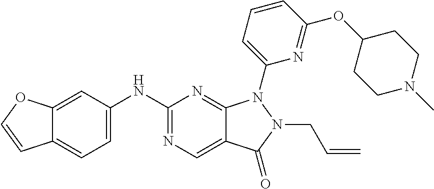 [SMILES: C=CCn1c(=O)c2cnc(Nc3ccc4ccoc4c3)nc2n1-c1cccc(OC2CCN(C)CC2)n1] | 498.0 | 1H NMR (500 MHz, DMSO) δ 10.49 (s, 1H), 9.41 (d, J = 28.6 Hz, 1H), 8.93 (s, 1H), 8.25 (s, 1H), 8.00 (dt, J = 11.9, 7.9 Hz, 1H), 7.94 (d, J = 2.2 Hz, 1H), 7.58 (d, J = 8.4 Hz, 1H), 7.53 (d, J = 7.7 Hz, 1H), 7.49 (dd, J = 8.4, 1.6 Hz, 1H), 6.91 (d, J = 2.4 Hz, 1H), 6.88 (dd, J = 9.9, 8.1 Hz, 1H), 5.72 (ddt, J = 16.7, 12.3, 5.3 Hz, 1H), 5.32-5.10 (m, 1H), 5.08 (ddd, J = 10.4, 6.3, 1.5 Hz, 1H), 4.96 (dt, J = 17.1, 1.5 Hz, 1H), 4.61 (t, J = 6.8 Hz, 2H), 3.49 (d, J = 12.4 Hz, 1H), 3.39- |
| | | | 3.30 (m, 1H), 3.17 (td, J = 12.8, 9.3 Hz, |
| | | | 2H), 2.81 (dd, J = 15.8, 4.7 Hz, 3H), |
| | | | 2.29 (d, J = 13.4 Hz, 1H), 2.14 (d, J = |
| | | | 14.9 Hz, 1H), 2.02 (t, J = 14.0 Hz, 1H), |
| | | | 1.80 (qd, J = 14.3, 3.6 Hz, 1H). |
| 309 | 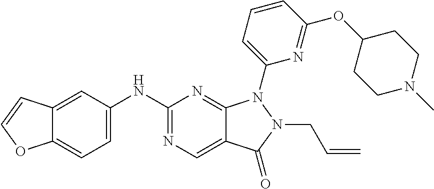 [SMILES: C=CCn1c(=O)c2cnc(Nc3ccc4occc4c3)nc2n1-c1cccc(OC2CCN(C)CC2)n1] | 498.0 | 1H NMR (500 MHz, DMSO) δ 10.39 (s, 1H), 9.51 (d, J = 35.6 Hz, 1H), 8.89 (s, 1H), 8.13 (s, 1H), 8.06-8.01 (m, 1H), 7.99 (t, J = 2.0 Hz, 1H), 7.56 (s, 2H), 7.53 (dd, J = 7.7, 2.5 Hz, 1H), 6.96 (dd, J = 3.7, 2.2 Hz, 1H), 6.84 (dd, J = 11.3, 8.1 Hz, 1H), 5.77-5.66 (m, 1H), 5.30- 5.04 (m, 2H), 4.95 (dp, J = 17.2, 1.5 Hz, 1H), 4.62 (t, J = 6.9 Hz, 2H), 3.49 (d, J = 12.4 Hz, 1H), 3.35 (d, J = 12.2 Hz, 1H), 3.18 (qd, J = 12.0, 8.3 Hz, 2H), 2.81 (dd, J = 13.7, 4.6 Hz, 3H), 2.27 (d, |
| | | | J = 13.4 Hz, 1H), 2.12 (d, J = 14.9 Hz, |
| | | | 1H), 2.02 (t, J = 14.2 Hz, 1H), 1.85- |
| | | | 1.74 (m, 1H). |
| 310 | 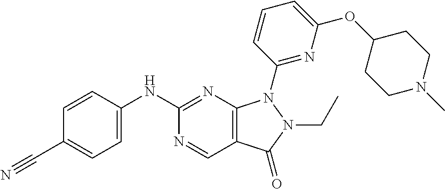 [SMILES: CCn1c(=O)c2cnc(Nc3ccc(C#N)cc3)nc2n1-c1cccc(OC2CCN(C)CC2)n1] | 471.0 | 1H NMR (400 MHz, DMSO-d6) δ 10.72 (s, 1H), 8.95 (s, 1H), 8.05 (t, J = 7.9 Hz, 1H), 7.95 (d, J = 8.8 Hz, 2H), 7.78 (d, J = 8.8 Hz, 2H), 7.48 (d, J = 7.7 Hz, 1H), 6.83 (d, J = 8.2 Hz, 1H), 4.96- 4.85 (m, 1H), 4.00 (q, J = 6.8 Hz, 2H), 2.65-2.56 (m, 2H), 2.21-2.05 (m, 5H), 2.00-1.84 (m, 2H), 1.71-1.60 (m, 2H), 1.03 (t, J = 7.0 Hz, 3H). |
| 311 | 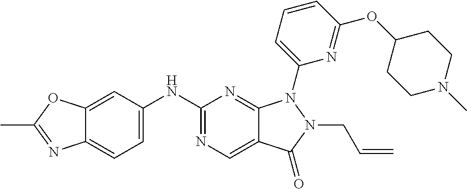 | 513.0 | 1H NMR (400 MHz, dmso) δ 10.53 (s, 1H), 9.42 (d, J = 20.7 Hz, 1H), 8.93 (s, 1H), 8.22 (s, 1H), 8.00 (q, J = 8.2 Hz, 1H), 7.61-7.53 (m, 2H), 7.50 (d, J = 7.7 Hz, 1H), 6.87 (t, J = 7.4 Hz, 1H), 5.78-5.64 (m, 1H), 5.29-5.04 (m, 2H), 4.94 (d, J = 17.2 Hz, 1H), 4.67- 4.54 (m, 2H), 3.49 (d, J = 11.9 Hz, 1H), 3.35 (d, J = 10.6 Hz, 1H), 3.17 (q, J = 10.5 Hz, 2H), 2.81 (dd, J = 13.2, 4.5 Hz, 3H), 2.60 (s, 3H), 2.26 (d, 1H), 2.18- 1.94 (m, 2H), 1.79 (q, J = 11.1 Hz, 1H). |
| 312 | 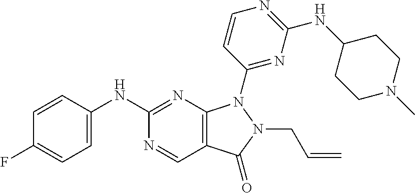 [SMILES: C=CCn1c(=O)c2cnc(Nc3ccc(F)cc3)nc2n1-c1ccnc(NC2CCN(C)CC2)n1] | 476.0 | 1H NMR (400 MHz, DMSO) δ 10.44 (s, 1H), 8.90 (s, 1H), 8.37 (d, J = 5.6 Hz, 1H), 7.74 (dd, J = 8.8, 5.0 Hz, 2H), 7.40 (d, J = 7.9 Hz, 1H), 7.23 (t, J = 8.7 Hz, 3H), 5.68 (d, J = 6.4 Hz, 1H), 5.06 (d, J = 10.3 Hz, 1H), 4.96 (d, J = 17.1 Hz, 1H), 4.79 (d, J = 6.1 Hz, 2H), 3.67 (s, 1H), 2.75 (d, J = 11.2 Hz, 2H), 2.16 (s, 3H), 1.94 (t, J = 11.3 Hz, 2H), 1.81 (d, J = 12.3 Hz, 2H), 1.52 (q, J = 10.2 Hz, 2H). |
| 313 | 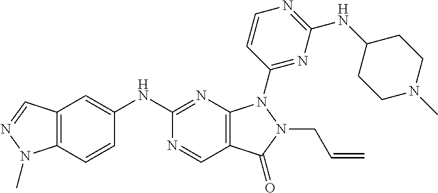 [SMILES: C=CCn1c(=O)c2cnc(Nc3ccc4c(cnn4C)c3)nc2n1-c1ccnc(NC2CCN(C)CC2)n1] | 512.0 | 1H NMR (400 MHz, DMSO) δ 10.50 (s, 1H), 8.89 (s, 1H), 8.37 (s, 1H), 8.18 (s, 1H), 8.06 (s, 1H), 7.64 (s, 2H), 7.40 (s, 1H), 7.23 (s, 1H), 5.78-5.57 (m, 1H), 5.06 (d, J = 10.2 Hz, 1H), 4.97 (d, J = 17.1 Hz, 1H), 4.79 (d, J = 6.1 Hz, 2H), 4.05 (s, 3H), 3.68 (s, 1H), 2.75 (d, J = 11.1 Hz, 2H), 2.16 (s, 3H), 1.95 (s, 2H), 1.82 (d, J = 12.1 Hz, 2H), 1.52 (d, J = 12.4 Hz, 2H). |
| 314 | 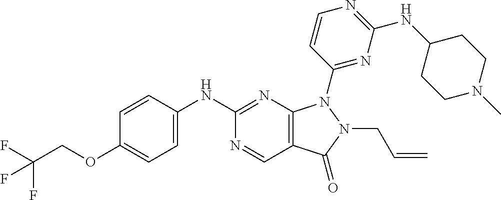 [SMILES: C=CCn1c(=O)c2cnc(Nc3ccc(OCC(F)(F)F)cc3)nc2n1-c1ccnc(NC2CCN(C)CC2)n1] | 556.0 | 1H NMR (500 MHz, DMSO) δ 10.37 (s, 1H), 8.87 (s, 1H), 8.46-8.29 (m, 1H), 7.68 (d, J = 8.5 Hz, 2H), 7.38 (s, 1H), 7.29-6.98 (m, 3H), 5.69 (ddd, J = 22.3, 10.8, 5.8 Hz, 1H), 5.07 (d, J = 10.2 Hz, 1H), 4.98 (d, J = 17.1 Hz, 1H), 4.80 (d, J = 6.2 Hz, 2H), 4.76 (q, J = 8.9 Hz, 2H), 3.69 (s, 1H), 2.76 (d, J = 11.1 Hz, 2H), 2.17 (s, 3H), 1.95 (d, J = 11.8 Hz, 2H), 1.82 (d, J = 12.3 Hz, 2H), 1.53 (d, J = 12.3 Hz, 2H). |
| 315 | 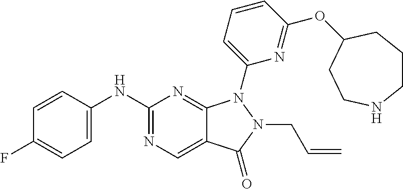 [SMILES: C=CCn1c(=O)c2cnc(Nc3ccc(F)cc3)nc2n1-c1cccc(OC2CCCNCC2)n1] | 476.0 | 1H NMR 1H NMR (400 MHz, cd3od) δ 8.83 (s, 1H), 7.92 (t, J = 7.9 Hz, 1H), 7.71-7.60 (m, 2H), 7.42 (d, J = 7.6 Hz, 1H), 7.05 (t, J = 8.8 Hz, 2H), 6.82 (d, J = 8.2 Hz, 1H), 5.81-5.67 (m, 1H), 5.42-5.32 (m, 1H), 5.13-4.97 (m, 2H), 4.69 (s, 2H), 3.46-3.34 (m, 1H), 3.5- 3.1 (m, 4H), 2.28-2.22 (m, 2H), 2.07 (m, 4H), 1.86 (dd, J = 12.6, 4.4 Hz, 1H). |
| 316 | 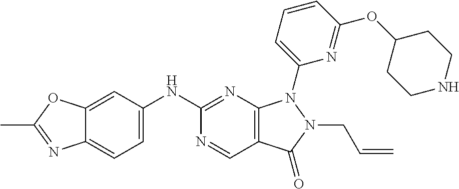 [SMILES: C=CCn1c(=O)c2cnc(Nc3ccc4nc(C)oc4c3)nc2n1-c1cccc(OC2CCNCC2)n1] | 499.0 | 1H NMR (400 MHz, dmso) δ 10.54 (s, 1H), 8.93 (s, 1H), 8.45 (d, J = 24.4 Hz, 2H), 8.22 (s, 1H), 8.00 (t, J = 7.9 Hz, 1H), 7.62-7.53 (m, 2H), 7.50 (d, J = 7.7 Hz, 1H), 6.88 (d, J = 8.1 Hz, 1H), 5.82-5.62 (m, 1H), 5.24-5.14 (m, 1H), 5.06 (d, J = 10.3 Hz, 1H), 4.94 (d, J = 17.2 Hz, 1H), 4.60 (s, 2H), 3.29-3.19 (m, 2H), 3.18-3.07 (m, 2H), 2.60 (s, 3H), 2.20-2.03 (m, 2H), 1.97-1.80 (m, 2H). |
| 317 | 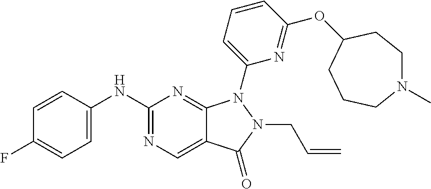 [SMILES: C=CCn1c(=O)c2cnc(Nc3ccc(F)cc3)nc2n1-c1cccc(OC2CCCN(C)CC2)n1] | 490.0 | 1H NMR 1H NMR (400 MHz, dmso) δ 10.35 (s, 1H), 9.5 (d, 1H), 8.77 (s, 1H), 8.00 (t, J = 7.1 Hz, 1H), 7.74 (dd, J = 8.9, 5.0 Hz, 2H), 7.43 (d, J = 7.7 Hz, 1H), 7.18 (t, J = 8.9 Hz, 2H), 6.79 (d, J = 8.2 Hz, 1H), 5.70 (ddt, J = 16.5, 10.9, 5.5 Hz, 1H), 5.24 (d, J = 4.6 Hz, 1H), 5.06 (d, J = 10.3 Hz, 1H), 4.93 (d, J = 17.2 Hz, 1H), 4.60 (s, 2H), 3.5-2.9 (m, 6H), 2.91-2.75 (m, 3H), 2.4-1.5 (m, 5H). |
| 318 | 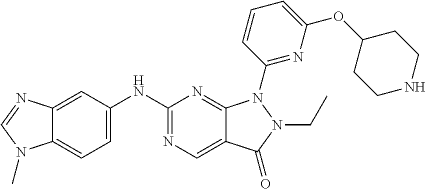 [SMILES: CCn1c(=O)c2cnc(Nc3ccc4c(c3)ncn4C)nc2n1-c1cccc(OC2CCNCC2)n1] | 486.4 | 1H-NMR (400 MHz, DMSO-d6): δ 10.29 (s, 1H), 8.85 (s, 1H), 8.13 (d, J = 12.80 Hz, 2H), 7.94 (t, J = 8.00 Hz, 1H), 7.57-7.49 (m, 3H), 6.81 (d, J = 8.40 Hz, 1H), 4.99-4.95 (m, 1H), 4.02-4.01 (m, 2H), 3.83 (s, 3H), 2.97-2.92 (m, 2H), 2.68 (t, J = 2.00 Hz, 2H), 1.93 (t, J = 5.20 Hz, 2H), 1.52-1.49 (m, 2H), 1.03 (t, J = 7.20 Hz, 3H). |
| 319 | 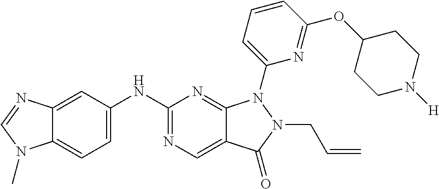 [SMILES: C=CCn1c(=O)c2cnc(Nc3ccc4c(c3)ncn4C)nc2n1-c1cccc(OC2CCNCC2)n1] | 498.44 | 1H-NMR (400 MHz, DMSO-d6): δ 10.35 (s, 1H), 8.88 (s, 1H), 8.14 (d, J = 4.80 Hz, 2H), 7.91 (t, J = 8.00 Hz, 1H), 7.58-7.50 (m, 3H), 6.79 (d, J = 8.00 Hz, 1H), 5.72 (q, J = 4.80 Hz, 1H), 5.08- 4.98 (m, 3H), 4.63 (d, J = 3.60 Hz, 3H), 3.83 (s, 3H), 2.93 (t, J = 3.60 Hz, 2H), 1.92 (t, J = 4.00 Hz, 2H), 1.54-1.46 (m, 2H). |
| 320 | 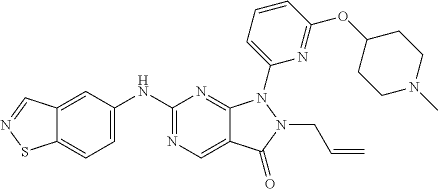 [SMILES: C=CCn1c(=O)c2cnc(Nc3ccc4sncc4c3)nc2n1-c1cccc(OC2CCN(C)CC2)n1] | 515 | 1H NMR: (400 MHz, dmso) δ 10.64 (1H, s), 9.36 (s, 1H), 9.07 (d, J = 3.4 Hz, 1H), 8.96 (s, 1H), 8.76 (s, 1H), 8.12 (dd, J = 22.1, 8.8 Hz, 2H), 7.81 (d, J = 9.0 Hz, 1H), 7.58 (dd, J = 7.4, 3.5 Hz, 1H), 6.86 (t, J = 8.1 Hz, 1H), 5.72 (dq, J = 16.7, 5.6 Hz, 1H), 5.31-5.02 (m, 1H), 4.94 (d, J = 17.1 Hz, 1H), 4.62 (s, 2H), 3.5-3.1 (m, 4H), 2.80 (dd, J = 10.9, 4.6 Hz, 3H), 2.31-1.67 (m, 4H). |
| 321 | 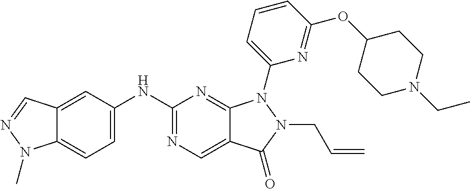 [SMILES: C=CCn1c(=O)c2cnc(Nc3ccc4c(cnn4C)c3)nc2n1-c1cccc(OC2CCN(CC)CC2)n1] | 526 | 1H NMR: 1H NMR (400 MHz, dmso) δ 10.43 (s, 1H), 9.25 (s, 1H), 8.89 (s, 1H), 8.26 (s, 1H), 8.01 (q, J = 9.6 Hz, 2H), 7.60 (s, 2H), 7.54 (d, J = 7.7 Hz, 1H), 6.84 (t, J = 8.4 Hz, 1H), 5.84-5.62 (m, 1H), 5.07 (dt, J = 10.4, 6.2 Hz, 1H), 4.94 (d, J = 17.1 Hz, 1H), 4.62 (s, 2H), 4.03 (s, 3H), 3.40 (d, J = 12.3 Hz, 2H), 3.14 (dt, J = 12.5, 7.1 Hz, 4H), 2.35- 2.25 (m, 2H), 2.21-1.71 (m, 3H), 1.22 (t, J = 6.8 Hz, 3H). |
| 322 | 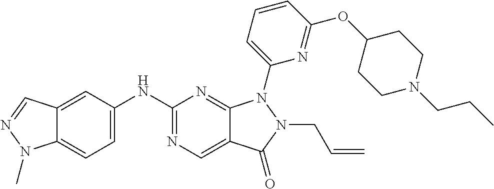 [SMILES: C=CCn1c(=O)c2cnc(Nc3ccc4c(cnn4C)c3)nc2n1-c1cccc(OC2CCN(CCC)CC2)n1] | 540.0 | 1H NMR: 1H NMR (400 MHz, dmso) δ 10.42 (s, 1H), 9.15 (s, 1H), 8.89 (s, 1H), 8.26 (s, 1H), 8.15-7.94 (m, 2H), 7.60 (s, 2H), 7.54 (d, J = 7.7 Hz, 1H), 6.84 (t, J = 7.7 Hz, 1H), 5.71 (dq, J = 15.9, 4.9 Hz, 1H), 5.33-5.03 (m, 1H), 4.94 (d, J = 17.1 Hz, 1H), 4.62 (s, 2H), 4.03 (s, 3H), 3.52 (s, 2H), 3.17-2.97 (m, 4H), 2.28 (d, J = 12.3 Hz, 1H), 2.06 (d, J = 26.3 Hz, 2H), 1.87-1.55 (m, 3H), 0.91 (t, J = 7.3 Hz, 3H). |
| 323 | 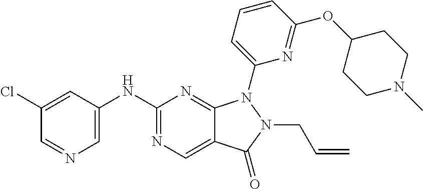 [SMILES: C=CCn1c(=O)c2cnc(Nc3cncc(Cl)c3)nc2n1-c1cccc(OC2CCN(C)CC2)n1] | 493.6 | 1H NMR: 1H NMR (400 MHz, dmso) δ 10.71 (s, 1H), 8.98 (s, 1H), 8.71 (d, J = 1.7 Hz, 1H), 8.54 (s, 1H), 8.26 (d, J = 2.1 Hz, 1H), 7.94 (t, J = 7.9 Hz, 1H), 7.41 (d, J = 7.6 Hz, 1H), 6.83 (d, J = 8.2 Hz, 1H), 5.71 (ddt, J = 16.1, 11.0, 5.7 Hz, 1H), 5.05 (d, J = 10.3 Hz, 1H), 4.92 (d, J = 16.1 Hz, 2H), 4.61 (d, J = 5.6 Hz, 2H), 2.58 (m, 4H), 2.16 (s, 3H), 2.00- 1.58 (m, 4H). |
| 324 | 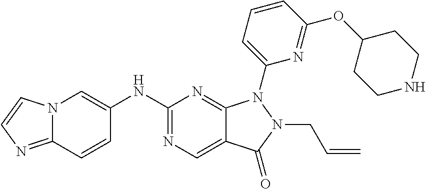 [SMILES: C=CCn1c(=O)c2cnc(Nc3ccc4nccn4c3)nc2n1-c1cccc(OC2CCNCC2)n1] | 484.0 | 1H NMR (400 MHz, dmso) δ 10.78 (s, 1H), 9.48 (s, 1H), 9.01 (s, 1H), 8.58 (d, J = 16.7 Hz, 2H), 8.29 (s, 1H), 8.15 (d, J = 1.9 Hz, 1H), 8.07 (t, J = 7.6 Hz, 1H), 8.01 (d, J = 11.5 Hz, 1H), 7.95 (d, J = 9.7 Hz, 1H), 7.66-7.55 (m, 1H), 6.88 (d, J = 8.2 Hz, 1H), 5.82-5.63 (m, 1H), 5.22-5.13 (m, 1H), 5.07 (d, J = 10.3 Hz, 1H), 4.93 (d, J = 18.3 Hz, 1H), 4.63 (d, J = 5.4 Hz, 2H), 3.29-3.20 (m, 2H), 3.19-3.07 (m, 2H), 2.16-2.04 (m, 2H), 1.92-1.77 (m, 2H). |
| 325 | 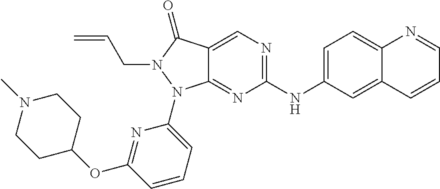 [SMILES: C=CCn1c(=O)c2cnc(Nc3ccc4ncccc4c3)nc2n1-c1cccc(OC2CCN(C)CC2)n1] | 509.0 | 1H NMR (400 MHz, cd3od) δ 8.96 (s, 1H), 8.87 (d, J = 3.6 Hz, 1H), 8.70 (s, 1H), 8.53 (d, J = 7.8 Hz, 1H), 8.15- 8.00 (m, 3H), 7.76 (dd, J = 8.4, 4.8 Hz, 1H), 7.59 (t, J = 8.4 Hz, 1H), 6.94 (dd, J = 31.3, 8.2 Hz, 1H), 5.78 (ddt, J = 16.4, 11.6, 5.9 Hz, 1H), 5.39 (s, 1H), 5.28- 5.08 (m, 1H), 5.01 (d, J = 17.1 Hz, 1H), 4.72 (d, J = 5.5 Hz, 2H), 3.62-3.08 (m, 4H), 2.90 (s, 3H), 2.34 (dd, J = 52.7, 14.5 Hz, 2H), 2.15-1.89 (m, 2H). |
| 326 | 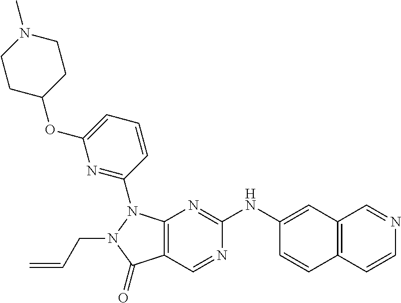 [SMILES: C=CCn1c(=O)c2cnc(Nc3ccc4ccncc4c3)nc2n1-c1cccc(OC2CCN(C)CC2)n1] | 509.0 | 1H NMR: 1H NMR (400 MHz, dmso) δ 10.72 (s, 1H), 9.12 (s, 1H), 8.98 (s, 1H), 8.67 (s, 1H), 8.40 (d, J = 5.6 Hz, 1H), 8.10-7.85 (m, 3H), 7.74 (d, J = 5.6 Hz, 1H), 7.55 (d, J = 7.6 Hz, 1H), 6.87 (d, J = 8.2 Hz, 1H), 5.73 (ddt, J = 16.2, 10.7, 5.7 Hz, 1H), 5.07 (d, J = 10.3 Hz, 1H), 5.00-4.86 (m, 2H), 4.62 (d, J = 5.6 Hz, 2H), 2.57 (s, 2H), 2.15 (s, 3H), 1.92 (s, 2H), 1.77-1.53 (m, 2H), 1.41-1.16 (m, 2H). |
| 327 | 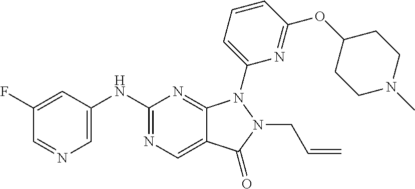 [SMILES: C=CCn1c(=O)c2cnc(Nc3cncc(F)c3)nc2n1-c1cccc(OC2CCN(C)CC2)n1] | 477.0 | 1H NMR (400 MHz, dmso) δ 10.73 (s, 1H), 9.51-9.31 (m, 1H), 9.00 (s, 1H), 8.69 (s, 1H), 8.33-8.19 (m, 2H), 8.05- 7.90 (m, 1H), 7.45 (dd, J = 7.7, 2.7 Hz, 1H), 6.97-6.84 (m, 1H), 5.81-5.63 (m, 1H), 5.30-5.04 (m, 2H), 4.94 (d, J = 17.3 Hz, 1H), 4.64-4.55 (m, 2H), 3.48 (d, J = 12.3 Hz, 1H), 3.35 (d, J = 11.3 Hz, 1H), 3.16 (q, J = 10.5 Hz, 2H), 2.81 (dd, J = 15.2, 4.7 Hz, 3H), 2.27 (d, J = 12.4 Hz, 1H), 2.12 (d, J = 15.6 Hz, 1H), 2.07-1.94 (m, 1H), 1.86-1.69 |
| | | | (m, 1H). |
| 328 | 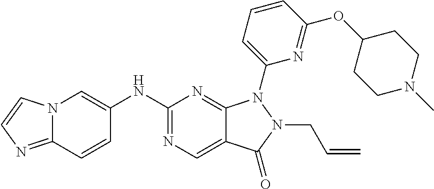 [SMILES: C=CCn1c(=O)c2cnc(Nc3ccc4nccn4c3)nc2n1-c1cccc(OC2CCN(C)CC2)n1] | 498.0 | 1H NMR (400 MHz, dmso) δ 10.78 (s, 1H), 9.57 (d, J = 31.3 Hz, 1H), 9.48 (s, 1H), 9.02 (s, 1H), 8.31 (s, 1H), 8.16 (d, J = 1.9 Hz, 1H), 8.12-7.94 (m, 3H), 7.62 (s, 1H), 6.92-6.82 (m, 1H), 5.81-5.64 (m, 1H), 5.30-5.04 (m, 2H), 4.94 (d, J = 17.9 Hz, 1H), 4.68-4.57 (m, 2H), 3.49 (d, J = 11.8 Hz, 1H), 3.35 (d, J = 11.1 Hz, 1H), 3.24-3.10 (m, 2H), 2.81 (d, J = 9.2 Hz, 3H), 2.25 (d, J = 13.1 Hz, 1H), 2.14-1.96 (m, 2H), 1.88-1.72 (m, 1H). |
| 329 | 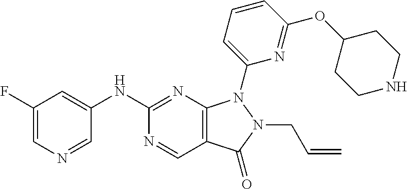 [SMILES: C=CCn1c(=O)c2cnc(Nc3cncc(F)c3)nc2n1-c1cccc(OC2CCNCC2)n1] | 463.3 | 1H NMR (400 MHz, dmso) δ 10.73 (s, 1H), 9.00 (s, 1H), 8.69 (s, 1H), 8.46 (d, J = 25.1 Hz, 2H), 8.30-8.25 (m, 1H), 8.23 (d, J = 2.6 Hz, 1H), 7.96 (t, J = 7.9 Hz, 1H), 7.44 (d, J = 7.5 Hz, 1H), 6.90 (d, J = 8.2 Hz, 1H), 5.79-5.64 (m, 1H), 5.24-5.13 (m, 1H), 5.06 (d, J = 10.4 Hz, 1H), 4.93 (d, J = 18.5 Hz, 1H), 4.60 (d, J = 5.6 Hz, 2H), 3.31-3.19 (m, 2H), 3.18-3.06 (m, 2H), 2.16-2.06 (m, 2H), 1.93-1.81 (m, 2H). |
| 330 | 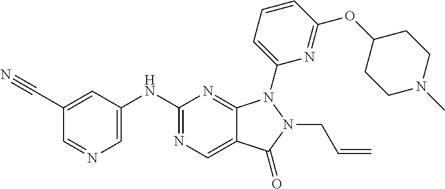 [SMILES: C=CCn1c(=O)c2cnc(Nc3cncc(C#N)c3)nc2n1-c1cccc(OC2CCN(C)CC2)n1] | 484.4 | 1H NMR (400 MHz, dmso) δ 10.83 (s, 1H), 9.04-8.93 (m, 2H), 8.82 (s, 1H), 8.65 (d, J = 1.8 Hz, 1H), 7.95 (t, J = 7.9 Hz, 1H), 7.46 (d, J = 7.6 Hz, 1H), 6.85 (d, J = 8.1 Hz, 1H), 5.80-5.62 (m, 1H), 5.06 (d, J = 10.2 Hz, 1H), 5.01-4.88 (m, 2H), 4.62 (d, J = 5.6 Hz, 2H), 2.69- 2.60 (m, 2H), 2.27-2.17 (m, 5H), 1.95 (d, J = 10.6 Hz, 2H), 1.69 (q, J = 9.2 Hz, 2H). Two exchangeable protons not listed. |
| 331 | 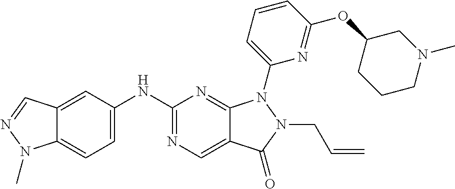 [SMILES: C=CCn1c(=O)c2cnc(Nc3ccc4c(cnn4C)c3)nc2n1-c1cccc(O[C@@H]2CCCN(C)C2)n1] | 512.0 | 1H NMR (400 MHz, dmso) δ 10.43 (s, 1H), 9.87-9.22 (m, 1H), 8.89 (s, 1H), 8.37-8.18 (m, 1H), 8.15-7.98 (m, 2H), 7.63-7.55 (m, 3H), 6.85 (d, J = 8.1 Hz, 1H), 5.80-5.64 (m, 1H), 5.45- 5.18 (m, 1H), 5.07 (d, J = 10.7 Hz, 1H), 4.95 (d, J = 16.8 Hz, 1H), 4.67-4.54 (m, 2H), 4.03 (s, 3H), 3.62 (d, J = 11.9 Hz, 1H), 3.38 (d, J = 9.7 Hz, 1H), 3.29 (t, J = 12.0 Hz, 1H), 3.05-2.90 (m, 1H), 2.79 (dd, J = 25.3, 4.4 Hz, 3H), 2.20-1.86 (m, 2H), 1.82-1.49 (m, 2H). |
| 332 | 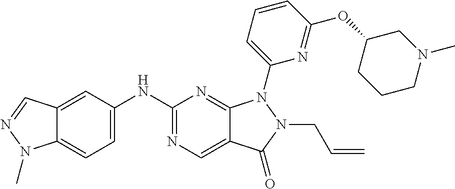 [SMILES: C=CCn1c(=O)c2cnc(Nc3ccc4c(cnn4C)c3)nc2n1-c1cccc(O[C@H]2CCCN(C)C2)n1] | 512.0 | 1H NMR (400 MHz, dmso) δ 10.44 (s, 1H), 9.85-9.25 (m, 1H), 8.89 (s, 1H), 8.38-8.19 (m, 1H), 8.18-7.99 (m, 2H), 7.67-7.51 (m, 3H), 6.85 (d, J = 8.1 Hz, 1H), 5.77-5.65 (m, 1H), 5.45- 5.17 (m, 1H), 5.07 (d, J = 10.5 Hz, 1H), 4.95 (d, J = 17.2 Hz, 1H), 4.69-4.53 (m, 2H), 4.03 (s, 3H), 3.62 (d, J = 12.7 Hz, 1H), 3.44-3.25 (m, 2H), 3.07- 2.90 (m, 1H), 2.84-2.72 (m, 3H), 2.21- 1.86 (m, 2H), 1.82-1.48 (m, 2H). |
| 333 | 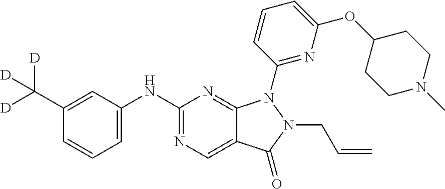 [SMILES: [2H]C([2H])([2H])c1cccc(Nc2ncc3c(=O)n(CC=C)n(-c4cccc(OC5CCN(C)CC5)n4)c3n2)c1] | 475.54 | 1H-NMR (400 MHz, DMSO-d6): δ 10.24 (s, 1H), 8.89 (s, 1H), 7.93 (t, J = 8.00 Hz, 1H), 7.70 (s, 1H), 7.46 (d, J = 7.60 Hz, 2H), 7.20 (t, J = 7.60 Hz, 1H), 6.83 (dd, J = 8.00, 27.00 Hz, 2H), 5.75- 5.67 (m, 1H), 4.93-5.04 (m, 3H), 4.62- 4.61 (m, 2H), 2.62-2.59 (m, 2H), 2.17- 1.97 (m, 5H), 1.96-1.93 (m, 2H), 1.89 (s, 2H), 1.72-1.67 (m, 2H) |
| 334 | 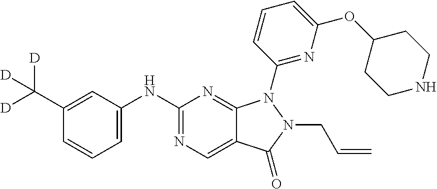 [SMILES: [2H]C([2H])([2H])c1cccc(Nc2ncc3c(=O)n(CC=C)n(-c4cccc(OC5CCNCC5)n4)c3n2)c1] | 461.51 | 1H-NMR (400 MHz, DMSO-d6): δ 10.25 (s, 1H), 8.89 (d, J = 3.60 Hz, 1H), 7.93 (t, J = 8.00 Hz, 1H), 7.70 (s, 1H), 7.45 (d, J = 7.60 Hz, 2H), 7.20 (t, J = 8.00 Hz, 1H), 6.83 (dd, J = 8.00, 27.80 Hz, 2H), 5.75-5.68 (m, 1H), 4.91-5.07 (m, 3H), 4.62-4.61 (m, 2H), 2.99-2.94 (m, 2H), 2.63-2.57 (m, 2H), 1.95-1.91 (m, 4H), 1.56-1.49 (m, 2H) |
| 335 | 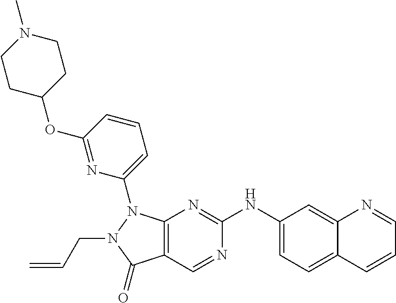 [SMILES: C=CCn1c(=O)c2cnc(Nc3ccc4cccnc4c3)nc2n1-c1cccc(OC2CCN(C)CC2)n1] | 509.0 | 1H NMR 1H NMR (400 MHz, dmso) δ 10.88 (s, 1H), 9.55 (d, J = 33.2 Hz, 1H), 9.15-8.92 (m, 2H), 8.71 (s, 1H), 8.54 (s, 1H), 8.14-7.94 (m, 3H), 7.62 (d, J = 7.9 Hz, 2H), 6.87 (t, J = 7.6 Hz, 1H), 5.74 (dq, J = 16.6, 5.4 Hz, 1H), 5.27 (s, 1H), 5.08 (dd, J = 10.3, 4.2 Hz, 1H), 4.96 (d, J = 17.1 Hz, 1H), 4.65 (t, J = 6.3 Hz, 2H), 3.49 (d, J = 12.4 Hz, 2H), 3.34 (d, J = 11.9 Hz, 2H), 2.80 (dd, J = 11.4, 4.3 Hz, 3H), 2.29-1.72 (m, 4H). |
| 336 | 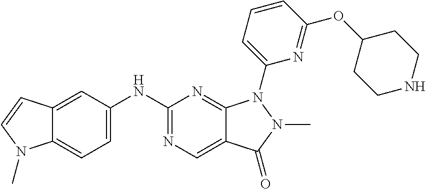 [SMILES: Cn1ccc2cc(Nc3ncc4c(=O)n(C)n(-c5cccc(OC6CCNCC6)n5)c4n3)ccc21] | 471.4 | 1H-NMR (400 MHz, DMSO-d6): δ 10.18 (br s, 1H), 8.82 (s, 1H), 8.04 (s, 1H), 7.94 (t, J = 8.00 Hz, 1H), 7.57 (d, J = 7.60 Hz, 1H), 7.39 (d, J = 8.40 Hz, 2H), 7.32 (d, J = 2.80 Hz, 1H), 6.77 (d, J = 8.00 Hz, 1H), 6.40 (d, J = 3.20 Hz, 1H), 5.01-4.97 (m, 1H), 3.79 (s, 3H), 3.42-3.35 (m, 3H), 2.96-2.93 (m, 2H), 2.58-2.56 (m, 2H), 1.94-1.91 (m, 2H), 1.82 (s, 2H), 1.54-1.45 (m, 2H) |
| 337 | 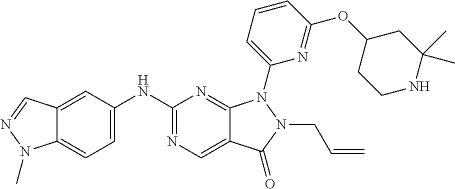 [SMILES: C=CCn1c(=O)c2cnc(Nc3ccc4c(cnn4C)c3)nc2n1-c1cccc(OC2CCNC(C)(C)C2)n1] | 526.56 | 1H-NMR (400 MHz, DMSO-d6,): δ 10.38 (s, 1H), 8.89 (s, 1H), 8.24 (br s, 1H), 8.01-7.97 (m, 2H), 7.64-7.58 (m, 2H), 7.48 (d, J = 7.60 Hz, 1H), 6.77 (d, J = 8.40 Hz, 1H), 5.74-5.68 (m, 1H), 5.16-5.15 (m, 1H), 5.11 (dd, J = 0.80, 19.80 Hz, 1H), 5.01 (dd, J = 17.20, 41.00 Hz, 1H), 4.62-4.63 (m, 2H), 4.04 (s, 3H), 2.86-2.77 (m, 2H), 1.93-1.88 (m, 2H), 1.42 (t, J = 6.80 Hz, 1H), 1.24 (t, J = 11.20 Hz, 1H), 1.05 (s, 6H). |
| 338 | 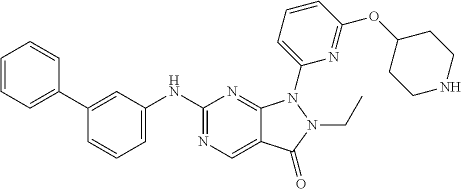 [SMILES: CCn1c(=O)c2cnc(Nc3cccc(-c4ccccc4)c3)nc2n1-c1cccc(OC2CCNCC2)n1] | 508.0 | 1H NMR: 1H NMR (400 MHz, dmso) δ 10.4 (s, 1H), 8.90 (s, 1H), 8.35 (s, 2H), 8.12 (s, 1H), 7.62 (s, 1H), 7.56 (d, J = 7.5 Hz, 2H), 7.48 (t, J = 7.5 Hz, 3H), 7.41 (t, J = 7.7 Hz, 2H), 7.31 (d, J = 7.5 Hz, 1H), 6.80 (d, J = 8.3 Hz, 1H), 5.12 (s, 1H), 3.96 (d, J = 7.1 Hz, 2H), 3.14 (d, J = 35.0 Hz, 4H), 2.06 (s, 2H), 1.83 (s, 2H), 1.00 (t, J = 7.0 Hz, 3H). |
| 339 | 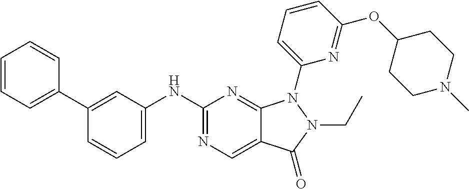 [SMILES: CCn1c(=O)c2cnc(Nc3cccc(-c4ccccc4)c3)nc2n1-c1cccc(OC2CCN(C)CC2)n1] | 522.0 | 1H NMR (400 MHz, dmso) δ 10.40 (s, 1H), 9.59 (d, J = 32.8 Hz, 1H), 8.90 (s, 1H), 8.11 (s, 1H), 7.88-7.08 (m, 9H), 6.83-6.73 (m, 1H), 5.3-5.0 (m, 1H), 3.97 (dd, J = 6.9, 3.3 Hz, 2H), 3.38 (dd, J = 57.8, 11.6 Hz, 2H), 3.22-3.02 (m, 2H), 2.79 (dd, J = 12.2, 4.4 Hz, 3H), 2.22 (d, J = 11.7 Hz, 1H), 2.02 (dt, J = 27.2, 14.2 Hz, 2H), 1.76 (q, J = 10.3 Hz, 1H), 1.00 (q, J = 6.8 Hz, 3H). |
| 340 | 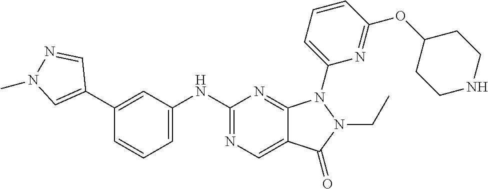 [SMILES: CCn1c(=O)c2cnc(Nc3cccc(-c4cnn(C)c4)c3)nc2n1-c1cccc(OC2CCNCC2)n1] | 512.0 | 1H NMR: 1H NMR (400 MHz, dmso) δ 10.28 (s, 1H), 8.89 (s, 1H), 8.43 (s, 1H), 7.97 (d, J = 20.2 Hz, 2H), 7.73 (s, 2H), 7.55 (d, J = 7.6 Hz, 1H), 7.46 (d, J = 7.7 Hz, 1H), 7.33-7.19 (m, 2H), 6.84 (d, J = 8.2 Hz, 1H), 5.14 (s, 1H), 3.97 (d, J = 7.1 Hz, 2H), 3.88 (s, 3H), 3.15 (d, J = 35.2 Hz, 4H), 2.07 (s, 2H), 1.85 (s, 2H), 1.00 (t, J = 7.0 Hz, 3H). |
| 341 | 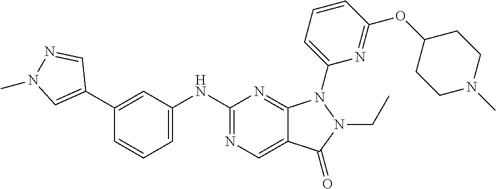 | 450.0 | 1H NMR (400 MHz, Methanol-d4) δ 8.92-8.72 (m, 1H), 8.54 (s, 1H), 8.13- 7.78 (m, 2H), 7.66-7.52 (m, 1H), 7.50 (d, J = 7.5 Hz, 1H), 6.95-6.76 (m, 1H), 5.27-5.09 (m, 1H), 4.09 (q, J = 7.0 Hz, 2H), 3.86 (s, 3H), 3.14-3.02 (m, 2H), 2.95-2.79 (m, 2H), 2.61 (s, 3H), 2.22- 2.07 (m, 2H), 2.06-1.93 (m, 2H), 1.12 (t, J = 7.0 Hz, 3H). |
| 342 | 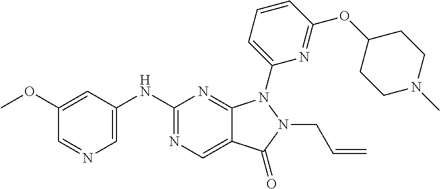 | 489.0 | 1H NMR (400 MHz, dmso) δ 10.58 (s, 1H), 9.49 (d, J = 27.8 Hz, 1H), 8.98 (s, 1H), 8.55 (s, 1H), 8.05 (d, J = 2.4 Hz, 1H), 8.02-7.89 (m, 2H), 7.45 (dd, J = 7.6, 3.0 Hz, 1H), 6.93-6.81 (m, 1H), 5.78-5.60 (m, 1H), 5.27-5.03 (m, 2H), 4.93 (d, J = 17.2 Hz, 1H), 4.60- 4.55 (m, 2H), 3.80 (d, J = 3.6 Hz, 3H), 3.48 (d, J = 11.7 Hz, 1H), 3.34 (d, J = 12.0 Hz, 1H), 3.16 (q, J = 10.5 Hz, 2H), 2.80 (dd, J = 15.3, 4.5 Hz, 3H), 2.26 (d, J = 11.7 Hz, 1H), 2.11 (d, J = 14.5 Hz, |
| | | | 1H), 2.06-1.95 (m, 1H), 1.85-1.71 |
| | | | (m, 1H). |
| 343 | 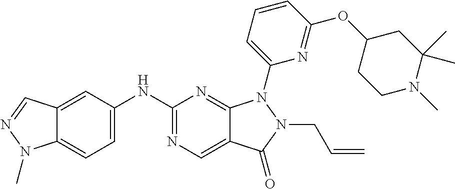 [SMILES: C=CCn1c(=O)c2cnc(Nc3ccc4c(cnn4C)c3)nc2n1-c1cccc(OC2CCN(C)C(C)(C)C2)n1] | 540.62 | 1H-NMR (400 MHz, DMSO-d6): δ 10.38 (s, 1H), 8.89 (s, 1H), 8.24 (br s, 1H), 8.01-7.97 (m, 2H), 7.64-7.58 (m, 2H), 7.48 (d, J = 8.00 Hz, 1H), 6.77 (d, J = 8.40 Hz, 1H), 5.76-5.69 (m, 1H), 5.08-4.94 (m, 3H), 4.63-4.62 (m, 2H), 4.04 (s, 3H), 2.62-2.58 (m, 1H), 2.48- 2.41 (m, 1H), 2.13 (s, 3H), 1.97-1.87 (m, 5H), 1.63-1.59 (m, 1H), 1.39 (t, J = 11.60 Hz, 1H), 1.06 (s, 3H), 0.93 (s, 1H). |
| 344 | 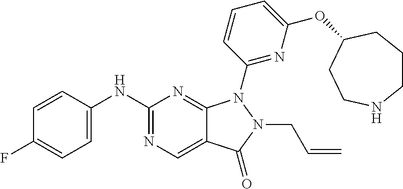 [SMILES: C=CCn1c(=O)c2cnc(Nc3ccc(F)cc3)nc2n1-c1cccc(O[C@@H]2CCCNCC2)n1] | 476.5 | 1H-NMR (400 MHz, DMSO-d6): δ 10.31 (br s, 1H), 8.89 (s, 1H), 7.98-7.94 (m, 1H), 7.77-7.73 (m, 2H), 7.43-7.38 (m, 1H), 7.18 (t, J = 8.80 Hz, 2H), 6.77 (d, J = 3.20 Hz, 1H), 5.74-5.67 (m, 1H), 5.16-5.14 (m, 2H), 5.06 (dd, J = 0.80, 10.00 Hz, 1H), 4.62 (d, J = 4.80 Hz, 1H), 2.82-2.78 (m, 3H), 2.05-1.97 (m, 2H), 1.96-1.80 (m, 4H) |
| 345 | 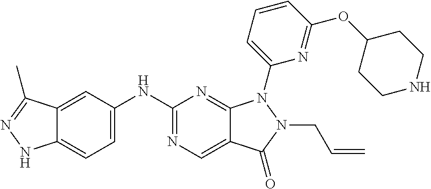 [SMILES: C=CCn1c(=O)c2cnc(Nc3ccc4[nH]nc(C)c4c3)nc2n1-c1cccc(OC2CCNCC2)n1] | 498.5 | 1H-NMR (400 MHz, DMSO-d6): δ 12.57 (br s, 1H), 10.33 (br s, 1H), 8.87 (s, 1H), 8.87 (br s, 1H), 7.89 (t, J = 8.00 Hz, 1H), 7.51-7.46 (m, 2H), 7.41 (d, J = 8.80 Hz, 1H), 6.79 (d, J = 8.00 Hz, 1H), 5.72-5.68 (m, 1H), 4.95-5.05 (m, 3H), 4.93 (s, 2H), 4.93 (d, J = 0.80 Hz, 2H), 2.52 (s, 2H), 2.44 (s, 3H), 2.44 (d, J = Hz, 2H), 1.50 (d, J = 9.20 Hz, 2H). |
| 346 | 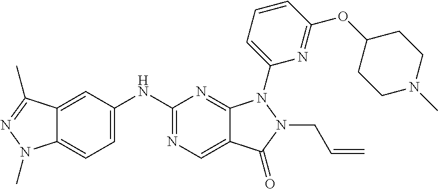 [SMILES: C=CCn1c(=O)c2cnc(Nc3ccc4c(c3)c(C)nn4C)nc2n1-c1cccc(OC2CCN(C)CC2)n1] | 526.5 | 1H-NMR (400 MHz, DMSO-d6): δ 10.38 (s, 1H), 8.88 (br s, 1H), 8.15 (br s, 1H), 7.90 (t, J = 8.00 Hz, 1H), 7.56-7.54 (m, 3H), 6.80 (d, J = 8.00 Hz, 1H), 5.69- 5.73 (m, 1H), 5.06 (d, J = 1.20 Hz, 1H), 4.94 (d, J = 4.40 Hz, 2H), 4.59 (s, 2H), 3.94 (s, 3H), 2.54 (d, J = 2.00 Hz, 2H), 2.44 (s, 3H), 2.13 (s, 5H), 1.93 (d, J = 10.40 Hz, 2H), 1.65-1.68 (m, 2H). |
| 347 | 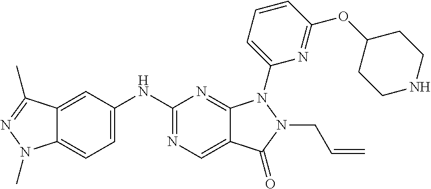 [SMILES: C=CCn1c(=O)c2cnc(Nc3ccc4c(c3)c(C)nn4C)nc2n1-c1cccc(OC2CCNCC2)n1] | 512.53 | 1H-NMR (400 MHz, DMSO-d6,): δ 10.34 (br s, 1H), 8.88 (br s, 1H), 8.15 (s, 1H), 7.90 (t, J = 8.00 Hz, 1H), 7.50-7.54 (m, 3H), 6.79 (d, J = 8.40 Hz, 1H), 5.75- 5.68 (m, 1H), 5.07-5.05 (m, 3H), 4.60 (s, 2H), 3.94 (s, 3H), 2.93 (d, J = 4.00 Hz, 2H), 2.52 (s, 2H), 2.42 (s, 3H), 1.91 (d, J = 4.00 Hz, 2H), 1.48 (d, J = 3.60 Hz, 2H). |
| 348 | 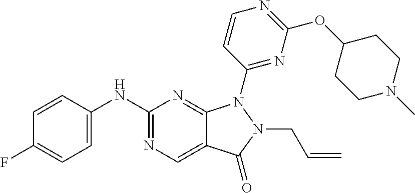 [SMILES: C=CCn1c(=O)c2cnc(Nc3ccc(F)cc3)nc2n1-c1ccnc(OC2CCN(C)CC2)n1] | 477.49 | 1H-NMR (400 MHz, DMSO-d6): δ 10.51 (s, 1H), 8.94 (s, 1H), 8.68 (d, J = 5.60 Hz, 1H), 7.76-7.73 (m, 3H), 7.28- 7.24 (m, 2H), 5.74-5.65 (m, 1H), 5.08- 4.97 (m, 3H), 4.76-4.74 (m, 2H), 2.68- 2.66 (m, 2H), 2.21 (s, 5H), 2.01-1.99 (m, 2H), 1.78-1.71 (m, 2H). |
| 349 | 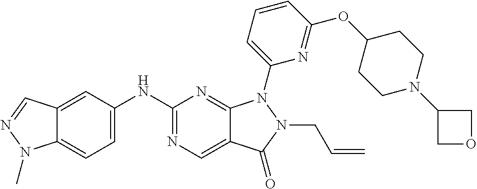 [SMILES: C=CCn1c(=O)c2cnc(Nc3ccc4c(cnn4C)c3)nc2n1-c1cccc(OC2CCN(C3COC3)CC2)n1] | 554.58 | 1H-NMR (400 MHz, DMSO-d6): δ 10.36 (br s, 1H), 8.88 (s, 1H), 8.23 (s, 1H), 8.02-7.98 (m, 2H), 7.63-7.58 (m, 2H), 7.49 (d, J = 8.00 Hz, 1H), 6.80 (d, J = 8.00 Hz, 1H), 5.74-5.67 (m, 1H), 5.07-5.05 (m, 3H), 5.00-4.92 (m, 2H), 4.62-4.61 (m, 2H), 4.54-4.40 (m, 2H), 4.04 (s, 3H), 3.42 (t, J = 6.40 Hz, 1H), 2.51-2.51 (m, 2H), 2.11-2.06 (m, 2H), 1.99-1.96 (m, 2H), 1.72-1.65 (m, 2H), |
| 350 | 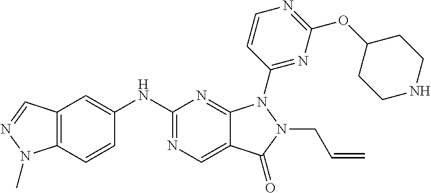 [SMILES: C=CCn1c(=O)c2cnc(Nc3ccc4c(cnn4C)c3)nc2n1-c1ccnc(OC2CCNCC2)n1] | 499.53 | 1H-NMR (400 MHz, DMSO-d6): δ 10.57 (br s, 1H), 8.93 (s, 1H), 8.65 (d, J = 6.00 Hz, 1H), 8.14-8.09 (m, 2H), 7.66 (br s, 3H), 5.65 (br s, 1H), 5.08-4.97 (m, 3H), 4.77-4.75 (m, 2H), 4.06 (s, 3H), 2.98 (s, 2H), 2.67-2.66 (m, 2H), 1.97 (s, 2H), 1.54 (s, 2H) |
| 351 | 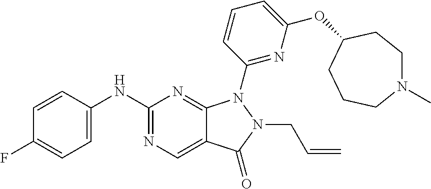 [SMILES: C=CCn1c(=O)c2cnc(Nc3ccc(F)cc3)nc2n1-c1cccc(O[C@H]2CCCN(C)CC2)n1] | 490.52 | 1H-NMR (400 MHz, DMSO-d6): δ 10.31 (br s, 1H), 8.89 (s, 1H), 7.95 (t, J = 7.60 Hz, 1H), 7.76-7.73 (m, 2H), 7.39 (d, J = 8.00 Hz, 1H), 7.18 (t, J = 9.20 Hz, 2H), 6.76 (d, J = 8.40 Hz, 1H), 5.72- 5.67 (m, 1H), 5.18-5.07 (m, 1H), 5.04 (d, J = 1.20 Hz, 1H), 4.95 (d, J = 0.80 Hz, 1H), 4.61 (d, J = 5.20 Hz, 2H), 2.67- 2.58 (m, 2H), 2.45-2.43 (m, 2H), 2.33 (s, 3H), 2.07-2.05 (m, 2H), 1.82-1.76 (m, 3H), 1.75-1.73 (m, 1H) |
| 352 | 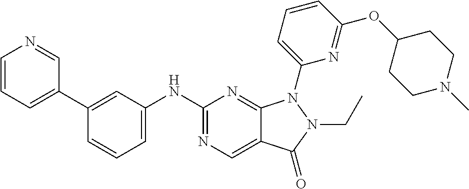 [SMILES: CCn1c(=O)c2cnc(Nc3cccc(-c4cccnc4)c3)nc2n1-c1cccc(OC2CCN(C)CC2)n1] | 523.4 | 1H NMR: 1H NMR (400 MHz, cd3od) δ 8.85 (s, 1H), 8.74 (s, 1H), 8.57 (d, J = 4.3 Hz, 1H), 8.14 (s, 1H), 7.98 (d, J = 7.8 Hz, 1H), 7.62 (d, J = 7.8 Hz, 1H), 7.53 (dd, J = 7.8, 4.9 Hz, 2H), 7.48- 7.30 (m, 3H), 6.72 (d, J = 8.2 Hz, 1H), 4.99 (s, 1H), 4.09 (q, J = 7.0 Hz, 2H), 2.68 (s, 2H), 2.32 (s, 2H), 2.27 (s, 3H), 1.98 (s, 2H), 1.78 (d, J = 8.8 Hz, 2H), 1.12 (t, J = 7.1 Hz, 3H). |
| 353 | 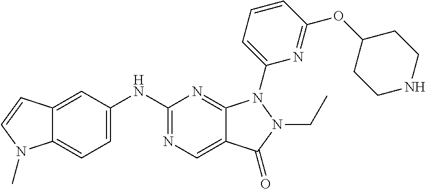 [SMILES: CCn1c(=O)c2cnc(Nc3ccc4c(ccn4C)c3)nc2n1-c1cccc(OC2CCNCC2)n1] | 485.51 | 1H-NMR (400 MHz, DMSO-d6): δ 10.22 (br s, 1H), 8.82 (s, 1H), 8.22 (s, 1H), 8.05-7.97 (m, 2H), 7.59 (d, J = 7.60 Hz, 1H), 7.38 (d, J = 8.40 Hz, 2H), 7.32 (d, J = 3.20 Hz, 1H), 6.84 (d, J = 8.00 Hz, 1H), 6.39 (d, J = 3.20 Hz, 1H), 5.12- 5.09 (m, 1H), 4.00 (s, 2H), 3.79 (s, 3H), 3.17-3.15 (m, 2H), 2.97-2.94 (m, 2H), 2.07-2.03 (m, 2H), 1.80-1.74 (m, 2H), 1.01 (t, J = 7.20 Hz, 3H) |
| 354 | 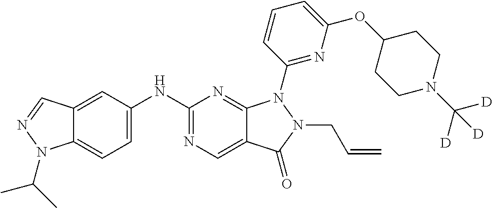 [SMILES: [2H]C([2H])([2H])N1CCC(Oc2cccc(-n3c4nc(Nc5ccc6c(cnn6C(C)C)c5)ncc4c(=O)n3CC=C)n2)CC1] | 543.0 | 1H NMR (400 MHz, DMSO) δ 10.42 (s, 1H), 9.53-9.26 (m, 1H), 8.88 (s, 1H), 8.27 (s, 1H), 8.03 (d, J = 2.4 Hz, 2H), 7.66 (d, J = 9.0 Hz, 1H), 7.56 (t, J = 7.6 Hz, 2H), 6.84 (t, J = 8.1 Hz, 1H), 5.71 (ddq, J = 16.3, 11.2, 5.8 Hz, 1H), 5.30- 5.02 (m, 2H), 5.00-4.88 (m, 2H), 4.62 (s, 2H), 3.48 (d, J = 12.3 Hz, 1H), 3.33 (d, J = 12.2 Hz, 1H), 3.15 (d, J = 11.1 Hz, 2H), 2.26 (d, J = 13.4 Hz, 1H), 2.11 (d, J = 14.7 Hz, 1H), 2.01 (t, J = 13.9 Hz, 1H), 1.78 (q, J = 11.7 Hz, 1H), 1.47 (d, J = 6.6 Hz, 6H). |
| 355 | 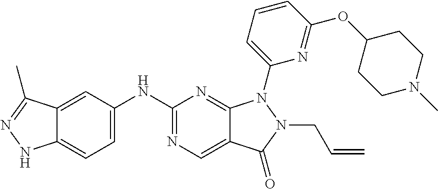 [SMILES: C=CCn1c(=O)c2cnc(Nc3ccc4[nH]nc(C)c4c3)nc2n1-c1cccc(OC2CCN(C)CC2)n1] | 512.53 | 1H-NMR (400 MHz, DMSO-d6): δ 12.56 (s,1H) 10.33 (s, 1H), 8.87 (s, 1H), 8.16 (s, 1H), 7.90 (d, J = 8.00 Hz, 1H), 7.46 (t, J = 8.80 Hz, 3H), 6.80 (d, J = 8.40 Hz, 1H), 5.70-5.66 (m, 1H), 5.06 (d, J = 1.20 Hz, 1H), 5.06 (d, J = 1.20 Hz, 2H), 4.59 (d, J = 5.60 Hz, 2H), 2.59- 2.58 (m, 2H), 2.44 (s, 3H), 2.16-2.12 (m, 5H), 1.95-1.85 (m, 2H), 1.70-1.68 (m, 2H). |
| 356 | 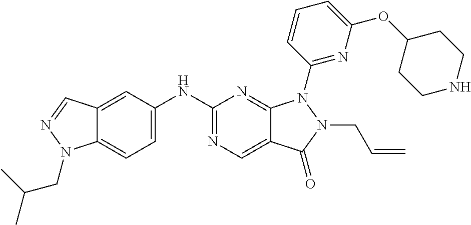 [SMILES: C=CCn1c(=O)c2cnc(Nc3ccc4c(cnn4CC(C)C)c3)nc2n1-c1cccc(OC2CCNCC2)n1] | 540.7 | 1H-NMR (400 MHz, DMSO-d6,): δ 10.36 (s, 1H), 8.88 (s, 1H), 8.23 (s, 1H), 8.03 (s, 1H), 7.97 (t, J = 7.60 Hz, 1H), 7.65-7.58 (m, 2H), 7.49 (d, J = 7.60 Hz, 1H), 6.79 (d, J = 8.00 Hz, 1H), 5.76- 5.67 (m, 1H), 5.06 (d, J = 9.60 Hz, 1H), 5.01-5.00 (m, 1H), 4.97 (dd, J = 16.80, 19.60 Hz, 1H), 4.63-4.62 (m, 2H), 4.20 (d, J = 7.20 Hz, 2H), 2.96-2.93 (m, 2H), 2.68-2.67 (m, 2H), 2.26-2.19 (m, 1H), 1.93-1.90 (m, 2H), 1.54-1.45 (m, 2H), 0.86 (d, J = 6.40 Hz, 6H). |
| 357 | 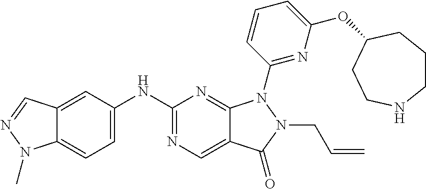 [SMILES: C=CCn1c(=O)c2cnc(Nc3ccc4c(cnn4C)c3)nc2n1-c1cccc(O[C@@H]2CCCNCC2)n1] | 512.57 | 1H-NMR (400 MHz, DMSO-d6): δ 8.89 (s, 1H), 8.25 (br s, 1H), 8.01-7.99 (m, 2H), 7.64-7.58 (m, 2H), 7.50-7.45 (m, 1H), 6.79-6.76 (m, 1H), 5.75-5.68 (m, 1H), 5.05-5.15 (m, 2H), 4.96-4.91 (m, 1H), 4.63 (s, 2H), 4.04 (s, 3H), 2.68- 2.53 (m, 3H), 2.05-1.94 (m, 2H), 1.74- 1.84 (m, 5H) |
| 358 | 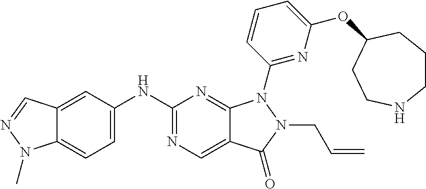 [SMILES: C=CCn1c(=O)c2cnc(Nc3ccc4c(cnn4C)c3)nc2n1-c1cccc(O[C@H]2CCCNCC2)n1] | 511.59 | 1H-NMR (400 MHz, DMSO-d6): δ 10.41 (br s, 1H), 8.89 (s, 1H), 8.25 (s, 1H), 8.01-7.96 (m, 2H), 7.63-7.58 (m, 2H), 7.50-7.45 (m, 1H), 6.77 (dd, J = 5.20, 8.20 Hz, 1H), 5.72-5.68 (m, 1H), 4.91-5.05 (m, 2H), 4.91-4.63 (m, 1H), 4.63 (s, 2H), 4.04 (s, 3H), 2.78-2.73 (m, 2H), 2.70-2.69 (m, 1H), 2.34-1.85 (m, 2H), 1.84-1.79 (m, 4H), 1.24 (m, 1H) |
| 359 | 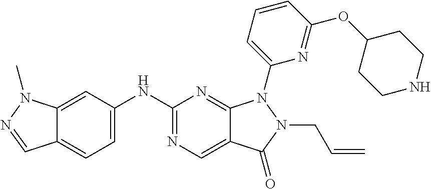 [SMILES: C=CCn1c(=O)c2cnc(Nc3ccc4cnn(C)c4c3)nc2n1-c1cccc(OC2CCNCC2)n1] | 498.5 | 1H-NMR (400 MHz, DMSO-d6): δ 10.52 (br s, 1H), 8.95 (s, 1H), 8.20 (s, 1H), 7.99-7.94 (m, 2H), 7.66 (d, J = 8.80 Hz, 1H), 7.53 (d, J = 7.60 Hz, 1H), 7.29 (d, J = 8.80 Hz, 1H), 6.83 (d, J = 8.00 Hz, 1H), 5.77-5.68 (m, 1H), 5.05-4.91 (m, 3H), 4.60 (d, J = 5.60 Hz, 2H), 3.94 (s, 3H), 2.95-2.93 (m, 2H), 2.68-2.67 (m, 2H), 1.93-1.91 (m, 3H), 1.54-1.52 (m, 2H) |
| 360 | 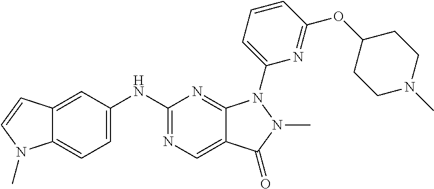 [SMILES: CN1CCC(Oc2cccc(-n3c4nc(Nc5ccc6c(ccn6C)c5)ncc4c(=O)n3C)n2)CC1] | 485.51 | 1H-NMR (400 MHz, DMSO-d6): δ 10.20 (br s, 1H), 8.83 (s, 1H), 8.04-7.93 (m, 2H), 7.57 (d, J = 7.60 Hz, 1H), 7.40- 7.32 (m, 3H), 6.78 (d, J = 8.00 Hz, 1H), 6.40 (d, J = 2.80 Hz, 1H), 4.96-4.95 (m, 1H), 3.79 (s, 3H), 3.42 (s, 3H), 2.68- 2.60 (m, 2H), 2.18-2.14 (m, 5H), 1.94- 1.91 (m, 2H), 1.69-1.67 (m, 2H) |
| 361 | 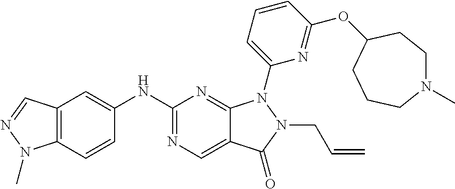 [SMILES: C=CCn1c(=O)c2cnc(Nc3ccc4c(cnn4C)c3)nc2n1-c1cccc(OC2CCCN(C)CC2)n1] | Cpd was not made | |
| 362 | 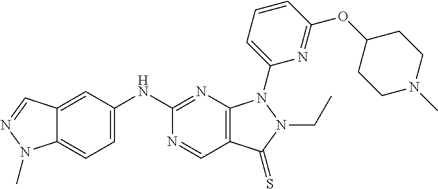 [SMILES: CCn1c(=S)c2cnc(Nc3ccc4c(cnn4C)c3)nc2n1-c1cccc(OC2CCN(C)CC2)n1] | 516.0 | 1H NMR (500 MHz, DMSO) δ 10.49 (s, 1H), 8.93 (s, 1H), 8.27 (s, 1H), 8.15 (s, 1H), 7.98 (s, 1H), 7.63-7.56 (m, 2H), 7.55 (d, J = 7.6 Hz, 1H), 6.99 (d, J = 8.2 Hz, 1H), 4.92 (tt, J = 8.4, 4.0 Hz, 1H), 4.52 (s, 2H, HSQC − CH2 13C: 41.8 ppm), 4.03 (s, 3H), 2.63-2.54 (m, 2H), 2.16 (s, 3H), 2.13 (d, J = 11.4 Hz, 2H), 1.92 (t, J = 8.7 Hz, 2H), 1.67 (dtd, J = 12.5, 8.7, 3.6 Hz, 2H), 1.16 (t, J = 7.0 Hz, 3H). |
| 363 | 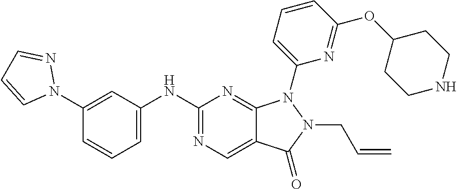 [SMILES: C=CCn1c(=O)c2cnc(Nc3cccc(-n4cccn4)c3)nc2n1-c1cccc(OC2CCNCC2)n1] | 510.0 | 1H NMR H NMR (400 MHz, dmso) δ 10.52 (s, 1H), 8.94 (s, 1H), 8.49 (s, 2H), 8.41 (d, J = 2.3 Hz, 2H), 7.80 (s, 1H), 7.71 (s, 1H), 7.57 (d, J = 7.6 Hz, 1H), 7.53-7.37 (m, 3H), 6.81 (d, J = 8.2 Hz, 1H), 6.59 (s, 1H), 5.71 (ddt, J = 16.3, 11.0, 5.8 Hz, 1H), 5.22-5.02 (m, 2H), 4.93 (d, J = 17.2 Hz, 1H), 4.61 (d, J = 5.5 Hz, 2H), 3.17 (d, J = 32.6 Hz, 4H), 2.15-1.72 (m, 4H). |
| 364 | 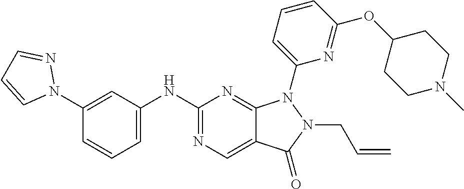 [SMILES: C=CCn1c(=O)c2cnc(Nc3cccc(-n4cccn4)c3)nc2n1-c1cccc(OC2CCN(C)CC2)n1] | 524.0 | 1H NMR (400 MHz, dmso) δ 10.53 (s, 1H), 9.51 (d, J = 29.0 Hz, 1H), 8.94 (s, 1H), 8.58-8.30 (m, 2H), 7.80 (s, 1H), 7.71 (s, 1H), 7.58 (dd, J = 7.5, 4.8 Hz, 1H), 7.53-7.38 (m, 3H), 6.80 (t, J = 7.6 Hz, 1H), 6.64-6.54 (m, 1H), 5.71 (ddt, J = 16.2, 10.7, 5.4 Hz, 1H), 5.31-5.01 (m, 2H), 4.94 (d, J = 7.1 Hz, 1H), 4.61 (t, J = 5.7 Hz, 2H), 3.33 (d, J = 11.8 Hz, 1H), 3.16 (q, J = 11.5 Hz, 2H), 2.80 (dd, J = 10.1, 4.5 Hz, 3H), 2.24 (d, J = 12.2 Hz, 1H), 2.15-1.95 (m, 2H), 1.77 (q, |
| | | | J = 10.3 Hz, 1H) |
| 365 | 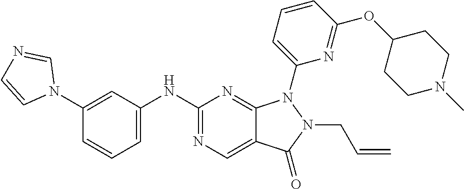 [SMILES: C=CCn1c(=O)c2cnc(Nc3cccc(-n4ccnc4)c3)nc2n1-c1cccc(OC2CCN(C)CC2)n1] | 524.0 | 1H NMR: 1H NMR (400 MHz, dmso) δ 10.67 (s, 1H), 9.74 (d, J = 42.9 Hz, 1H), 9.23 (s, 1H), 8.97 (s, 1H), 8.29 (s, 1H), 8.05 (s, 1H), 7.81-7.50 (m, 3H), 7.49- 7.35 (m, 2H), 6.79 (t, J = 8.2 Hz, 1H), 5.70 (ddd, J = 16.4, 10.6, 5.3 Hz, 1H), 5.32-5.00 (m, 2H), 4.91 (d, J = 17.3 Hz, 1H), 4.60 (s, 2H), 3.48 (d, J = 12.2 Hz, 1H), 3.34 (d, J = 11.4 Hz, 1H), 3.15 (s, 2H), 2.80 (d, J = 12.2 Hz, 3H), 2.23 (d, J = 12.3 Hz, 1H), 2.06 (t, J = 17.4 Hz, 2H), 1.86-1.71 (m, 1H). |
| 366 | 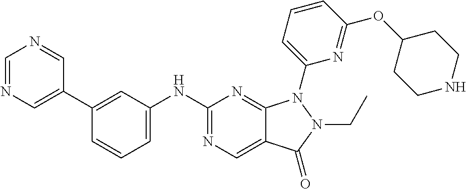 [SMILES: CCn1c(=O)c2cnc(Nc3cccc(-c4cncnc4)c3)nc2n1-c1cccc(OC2CCNCC2)n1] | 510.0 | 1H NMR: 1H NMR (400 MHz, dmso) δ 10.47 (s, 1H), 9.25 (s, 1H), 9.06 (s, 2H), 8.92 (s, 1H), 8.22 (d, J = 22.5 Hz, 1H), 7.69 (s, 2H), 7.54-7.42 (m, 3H), 6.78 (d, J = 8.1 Hz, 1H), 5.09 (s, 1H), 3.96 (q, J = 6.8 Hz, 2H), 3.15 (d, J = 7.2 Hz, 2H), 3.00 (t, J = 8.9 Hz, 2H), 2.03 (s, 2H), 1.77 (d, J = 9.1 Hz, 2H), 1.00 (t, J = 7.0 Hz, 3H). |
| 367 | 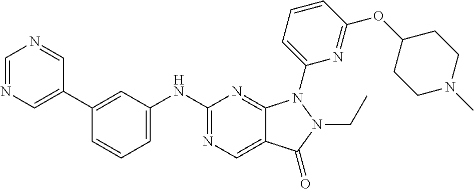 [SMILES: CCn1c(=O)c2cnc(Nc3cccc(-c4cncnc4)c3)nc2n1-c1cccc(OC2CCN(C)CC2)n1] | 524.0 | 1H NMR: 1H NMR (400 MHz, dmso) δ 10.48 (s, 1H), 9.54 (d, J = 28.0 Hz, 1H), 9.25 (s, 1H), 9.06 (d, J = 2.5 Hz, 2H), 8.92 (s, 1H), 8.25 (s, 1H), 7.76-7.55 (m, 2H), 7.55-7.41 (m, 3H), 6.82- 6.73 (m, 1H), 3.97 (dd, J = 6.7, 3.3 Hz, 2H), 3.32 (d, J = 11.7 Hz, 2H), 3.14 (d, J = 10.3 Hz, 2H), 2.79 (dd, J = 14.1, 4.1 Hz, 3H), 2.22 (d, J = 12.1 Hz, 1H), 2.03 (dt, J = 26.8, 14.1 Hz, 2H), 1.76 (q, J = 10.3 Hz, 1H), 1.00 (q, J = 6.8 Hz, 3H). |
| 368 | 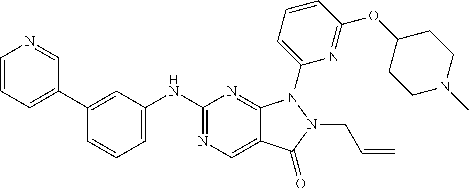 [SMILES: C=CCn1c(=O)c2cnc(Nc3cccc(-c4cccnc4)c3)nc2n1-c1cccc(OC2CCN(C)CC2)n1] | 535.0 | 1H NMR (400 MHz, dmso) δ 10.46 (s, 1H), 9.56 (d, J = 30.4 Hz, 1H), 8.93 (s, 1H), 8.56 (s, 1H), 7.91 (dt, J = 26.5, 7.7 Hz, 2H), 7.73 (d, J = 7.6 Hz, 2H), 7.55 (t, J = 6.7 Hz, 2H), 7.48-7.39 (m, 2H), 6.77 (d, J = 6.5 Hz, 1H), 5.71 (ddt, J = 16.2, 10.8, 5.4 Hz, 1H), 5.24 (s, 1H), 5.07 (dd, J = 9.8, 4.6 Hz, H), 4.94 (d, J = 17.2 Hz, 1H), 4.61 (s, 2H), 3.47 (d, J = 12.0 Hz, 1H), 3.32 (d, J = 11.9 Hz, 1H), 3.25-3.03 (m, 2H), 2.80 (dd, J = 10.5, 4.6 Hz, 3H), 2.24 (d, J = 11.3 Hz, 1H), |
| | | | 2.04 (dt, J = 27.9, 14.5 Hz, 2H), 1.77 (q, |
| | | | J = 10.4 Hz, 1H) |
| 369 | 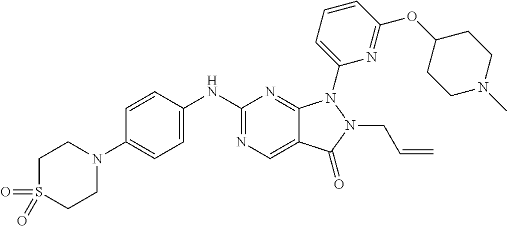 [SMILES: C=CCn1c(=O)c2cnc(Nc3ccc(N4CCS(=O)(=O)CC4)cc3)nc2n1-c1cccc(OC2CCN(C)CC2)n1] | 591.0 | 1H NMR (400 MHz, dmso) δ 10.25 (s, 1H), 9.50 (d, J = 28.8 Hz, 1H), 8.84 (s, 1H), 8.02 (s, 1H), 7.61 (s, 2H), 7.49 (d, J = 7.7 Hz, 1H), 7.02 (d, J = 8.8 Hz, 2H), 6.82 (t, J = 7.8 Hz, 1H), 5.70 (ddq, J = 16.3, 11.3, 5.7 Hz, 1H), 5.30-5.00 (m, 2H), 4.93 (d, J = 17.1 Hz, 1H), 4.61 (s, 2H), 3.73 (s, 4H), 3.49 (d, J = 11.8 Hz, 1H), 3.34 (d, J = 11.6 Hz, 1H), 3.19- 3.14 (m, 6H), 2.81 (dd, J = 11.2, 4.5 Hz, 3H), 2.26 (d, J = 12.5 Hz, 1H), 2.06 (dt, J = 27.5, 14.4 Hz, 2H), 1.79 (q, J = 10.2 Hz, 1H). |
| 370 | 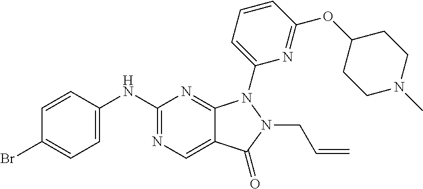 [SMILES: C=CCn1c(=O)c2cnc(Nc3ccc(Br)cc3)nc2n1-c1cccc(OC2CCN(C)CC2)n1] | 538.0 | 1H NMR: 1H NMR (400 MHz, dmso) δ 10.45 (s, 1H), 9.40 (s, 1H), 8.92 (s, 1H), 8.07 (d, J = 8.0 Hz, 1H), 7.74 (d, J = 8.7 Hz, 2H), 7.49 (dd, J = 19.5, 8.2 Hz, 3H), 6.84 (t, J = 7.9 Hz, 1H), 5.71 (ddd, J = 16.5, 10.6, 5.5 Hz, 1H), 5.31- 5.00 (m, 2H), 4.93 (d, J = 17.2 Hz, 1H), 4.61 (s, 2H), 3.33 (s, 2H), 3.16 (d, J = 8.9 Hz, 2H), 2.81 (dd, J = 12.4, 4.5 Hz, 3H), 2.26 (d, J = 11.9 Hz, 1H), 2.16- 1.94 (m, 2H), 1.89-1.69 (m, 1H). |
| 371 | 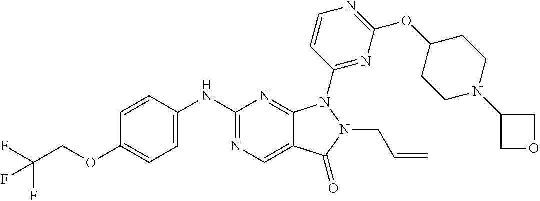 [SMILES: C=CCn1c(=O)c2cnc(Nc3ccc(OCC(F)(F)F)cc3)nc2n1-c1ccnc(OC2CCN(C3COC3)CC2)n1] | 599.3 | 1H-NMR (400 MHz, DMSO-d6): δ 10.42 (br s, 1H), 8.89 (s, 1H), 8.67 (d, J = 4.80 Hz, 1H), 7.73-7.63 (m, 3H), 7.11 (d, J = 7.60 Hz, 2H), 5.73-5.66 (m, 1H), 5.08-5.01 (m, 3H), 4.80-4.73 (m, 4H), 4.56-4.53 (m, 2H), 4.46-4.42 (m, 2H), 3.46-3.41 (m, 1H), 2.68-2.51 (m, 2H), 2.19-2.09 (m, 4H), 1.75 (d, J = 8.80 Hz, 2H) |
| 372 | 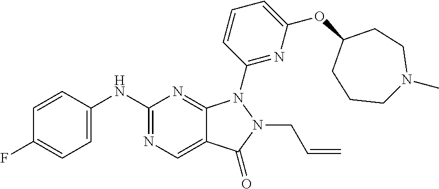 [SMILES: C=CCn1c(=O)c2cnc(Nc3ccc(F)cc3)nc2n1-c1cccc(O[C@@H]2CCCN(C)CC2)n1] | 490.49 | 1H-NMR (400 MHz, DMSO-d6): δ 10.32 (br s, 1H), 8.89 (s, 1H), 7.95 (t, J = 8.00 Hz, 1H), 7.76-7.73 (m, 2H), 7.39 (d, J = 7.60 Hz, 1H), 7.18 (t, J = 8.80 Hz, 2H), 6.76 (d, J = 8.00 Hz, 1H), 5.74- 5.67 (m, 1H), 5.19-5.15 (m, 1H), 5.07- 5.04 (m, 1H), 4.95-4.91 (m, 1H), 4.61 (d, J = 5.20 Hz, 2H), 2.56-2.51 (m, 2H), 2.45-2.44 (m, 1H), 2.25 (s, 3H), 2.05- 1.82 (m, 2H), 1.76-1.71 (m, 4H), 1.58- 1.55 (m, 1H) |
| 373 | 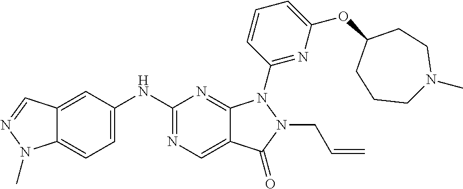 [SMILES: C=CCn1c(=O)c2cnc(Nc3ccc4c(cnn4C)c3)nc2n1-c1cccc(O[C@@H]2CCCN(C)CC2)n1] | 524.55 | 1H-NMR (400 MHz, DMSO-d6): δ 10.41 (br s, 1H), 8.89 (s, 1H), 8.24 (s, 1H), 8.00 (d, J = 11.60 Hz, 2H), 7.61 (t, J = 8.80 Hz, 2H), 7.46 (d, J = 7.60 Hz, 1H), 6.77 (d, J = 8.40 Hz, 1H), 5.76- 5.67 (m, 1H), 5.18-5.18 (m, 1H), 5.06 (d, J = 10.40 Hz, 1H), 4.93 (d, J = 17.20 Hz, 1H), 4.63 (s, 2H), 4.04 (s, 3H), 2.68-2.55 (m, 2H), 2.45-2.34 (m, 2H), 2.25 (s, 3H), 2.06-1.85 (m, 2H), 1.81- 1.75 (m, 3H), 1.57-1.56 (m, 1H) |
| 374 | 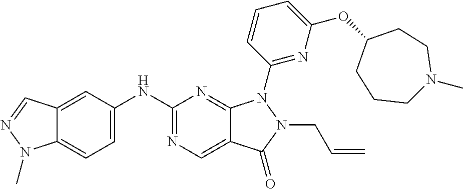 [SMILES: C=CCn1c(=O)c2cnc(Nc3ccc4c(cnn4C)c3)nc2n1-c1cccc(O[C@H]2CCCN(C)CC2)n1] | 526.56 | 1H-NMR (400 MHz, DMSO-d6): δ 10.38 (br s, 1H), 8.89 (s, 1H), 8.25 (s, 1H), 8.00 (d, J = 11.60 Hz, 2H), 7.61 (t, J = 8.80 Hz, 2H), 7.46 (d, J = 8.00 Hz, 1H), 6.77 (d, J = 8.00 Hz, 1H), 5.75- 5.68 (m, 1H), 5.17-5.16 (m, 1H), 5.07- 5.05 (m, 1H), 4.95-4.91 (m, 1H), 4.62 (s, 2H), 4.04 (s, 3H), 2.68-2.60 (m, 2H), 2.55-2.51 (m, 2H), 2.33 (s, 3H), 2.24- 2.09 (m, 2H), 2.01-1.81 (m, 3H), 1.77- 1.71 (m, 1H) |
| 375 | 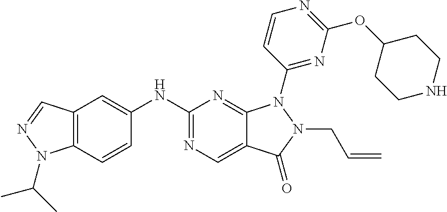 [SMILES: C=CCn1c(=O)c2cnc(Nc3ccc4c(cnn4C(C)C)c3)nc2n1-c1ccnc(OC2CCNCC2)n1] | 527.5 | 1H-NMR (400 MHz, DMSO-d6): δ 10.54 (br s, 1H), 8.93 (s, 1H), 8.65 (d, J = 5.60 Hz, 1H), 8.11 (br s, 2H), 7.74- 7.69 (m, 2H), 7.62 (m, 1H), 5.74-5.67 (m, 1H), 5.08-4.97 (m, 4H), 4.76 (br s, 2H), 2.98 (d, J = 3.60 Hz, 2H), 2.68- 2.59 (m, 2H), 1.99-1.97 (m, 2H), 1.89 (s, 1H), 1.57-1.44 (m, 8H) |
| 376 | 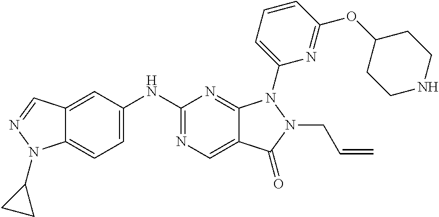 [SMILES: C=CCn1c(=O)c2cnc(Nc3ccc4c(cnn4C4CC4)c3)nc2n1-c1cccc(OC2CCNCC2)n1] | 524.5 | 1H-NMR (400 MHz, DMSO-d6): δ 10.4 (br s, 1H), δ 8.89 (s, 1H), 8.24 (br s, 1H), 7.98 (t, J = 8.40 Hz, 2H), 7.64 (t, J = 10.40 Hz, 2H), 7.49 (d, J = 7.60 Hz, 1H), 6.80 (d, J = 8.00 Hz, 1H), 5.79- 5.60 (m, 1H), 5.08-5.01 (m, 3H), 4.62 (br s, 2H), 3.75-3.73 (m, 1H), 2.99-2.95 (m, 2H), 2.68-2.62 (m, 2H), 1.95-1.91 (m, 4H), 1.56-1.53 (m, 2H), 1.13-1.10 (m, 4H). |
| 377 | 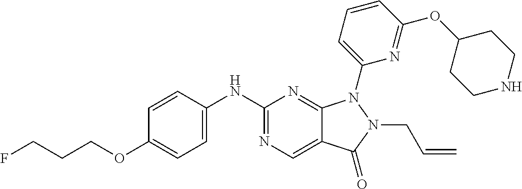 [SMILES: C=CCn1c(=O)c2cnc(Nc3ccc(OCCCF)cc3)nc2n1-c1cccc(OC2CCNCC2)n1] | 520.5 | 1H-NMR (400 MHz, DMSO-d6,): δ 10.21 (s, 1H), 8.85 (s, 1H), 8.29 (s, 1H), 7.97 (t, J = 8.00 Hz, 1H), 7.63 (d, J = 8.00 Hz, 2H), 7.44 (d, J = 7.60 Hz, 1H), 6.94 (d, J = 8.80 Hz, 2H), 6.79 (d, J = 8.40 Hz, 1H), 5.60-5.30 (m, 1H), 5.06 (dd, J = 3.60, 11.00 Hz, 2H), 4.94 (dd, J = 0.80, 17.20 Hz, 1H), 4.70-4.55 (m, 4H), 4.06 (t, J = 6.40 Hz, 2H), 3.07-3.02 (m, 3H), 2.76 (t, J = 9.60 Hz, 2H), 2.14- 1.98 (m, 4H), 1.65-1.62 (m, 2H). |
| 378 | 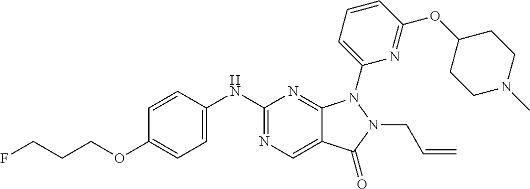 [SMILES: C=CCn1c(=O)c2cnc(Nc3ccc(OCCCF)cc3)nc2n1-c1cccc(OC2CCN(C)CC2)n1] | 532.3 | 1H-NMR (400 MHz, DMSO-d6): δ 9.88 (s, 1H), 8.80 (s, 1H), 8.17 (s, 1H), 7.91 (t, J = 8.00 Hz, 1H), 7.61 (d, J = 8.80 Hz, 2H), 7.42 (d, J = 7.60 Hz, 1H), 6.92 (d, J = 8.80 Hz, 2H), 6.77 (d, J = 8.00 Hz, 1H), 5.60-5.30 (m, 1H), 5.08-4.95 (m, 3H), 4.68 (t, J = 6.00 Hz, 1H), 4.60- 4.55 (m, 3H), 4.09 (t, J = 6.40 Hz, 2H), 2.64-2.61 (m, 2H), 2.51-2.20 (m, 6H), 2.18-2.15 (m, 1H), 2.09-1.94 (m, 2H), 1.73-1.70 (m, 2H). |
| 379 | 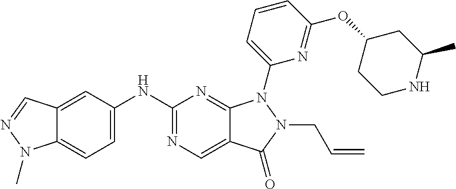 [SMILES: C=CCn1c(=O)c2cnc(Nc3ccc4c(cnn4C)c3)nc2n1-c1cccc(O[C@H]2CCN[C@H](C)C2)n1] | 526.3 | 1H-NMR (400 MHz, DMSO-d6): δ 10.35 (s, 1H), 8.88 (s, 1H), 8.24 (s, 1H), 8.02-7.97 (m, 2H), 7.60-7.58 (m, 2H), 7.48 (d, J = 8.00 Hz, 1H), 7.48 (d, J = 8.00 Hz, 1H), 5.74-5.68 (m, 1H), 5.13- 5.11 (m, 1H), 5.06 (d, J = 9.60 Hz, 1H), 4.93 (d, J = 17.20 Hz, 1H), 4.61 (d, J = 4.00 Hz, 2H), 4.04 (s, 3H), 2.36-2.32 (m, 2H), 2.18 (s, 3H), 1.84-1.76 (m, 11H), 1.57-1.51 (m, 1H), 0.97 (d, J = 6.00 Hz, 3H), (3 Acetic acid salt). |
| 380 | 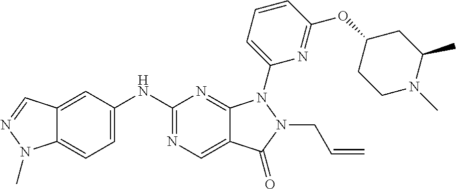 [SMILES: C=CCn1c(=O)c2cnc(Nc3ccc4c(cnn4C)c3)nc2n1-c1cccc(O[C@H]2CCN(C)[C@H](C)C2)n1] | 524.58 | 1H-NMR (400 MHz, DMSO-d6): δ 10.06 (s, 1H), 8.84 (s, 1H), 8.17-8.15 (m, 1H), 7.96-7.91 (m, 2H), 8.01-7.97 (m, 2H), 7.64 (dd, J = 1.60, 9.00 Hz, 1H), 7.55 (d, J = 8.80 Hz, 1H), 7.47 (d, J = 7.60 Hz, 1H), 6.77 (d, J = 8.00 Hz, 1H), 5.76-5.72 (m, 1H), 5.08 (dd, J = 1.20, 10.20 Hz, 2H), 5.02 (d, J = 1.20 Hz, 2H), 4.98-4.88 (m, 1H), 4.61 (d, J = 6.00 Hz, 2H), 4.04 (s, 3H), 2.85-2.82 (m, 1H), 2.19 (s, 3H), 2.15-2.12 (m, 1H), 2.09-2.01 (m, 3H), 1.70-1.64 (m, |
| | | | 1H), 1.36-1.27 (m, 1H), 1.03 (d, J = 6.00 |
| | | | Hz, 3H). |
| 381 | 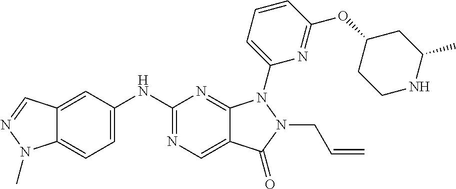 [SMILES: C=CCn1c(=O)c2cnc(Nc3ccc4c(cnn4C)c3)nc2n1-c1cccc(O[C@H]2CCN[C@@H](C)C2)n1] | 510.52 | 1H-NMR (400 MHz, DMSO-d6,): δ 10.39 (s, 1H), 8.89 (s, 1H), 8.27-8.24 (m, 2H), 8.01-7.98 (m, 2H), 7.64-7.58 (m, 2H), 7.50 (d, J = 7.60 Hz, 1H), 6.80 (d, J = 8.00 Hz, 1H), 5.76-5.70 (m, 1H), 5.08-4.93 (m, 3H), 4.62 (d, J = 8.00 Hz, 2H), 4.04 (s, 3H), 3.12-3.08 (m, 1H), 2.87-2.83 (m, 1H), 2.76-2.68 (m, 1H), 2.10-2.04 (m, 2H), 1.49-1.46 (m, 1H), 1.26-1.23 (m, 1H), 1.07 (d, J = 6.40 Hz, 3H). |
| 382 | 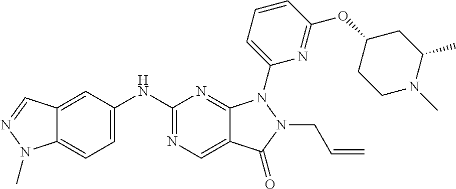 [SMILES: C=CCn1c(=O)c2cnc(Nc3ccc4c(cnn4C)c3)nc2n1-c1cccc(O[C@H]2CCN(C)[C@@H](C)C2)n1] | 524.58 | 1H-NMR (400 MHz, DMSO-d6): δ 10.37 (s, 1H), 8.89 (s, 1H), 8.23 (s, 1H), 8.17 (s, 1H), 8.01-7.97 (m, 2H), 7.64- 7.58 (m, 2H), 7.49 (d, J = 7.60 Hz, 1H), 6.78 (d, J = 8.00 Hz, 1H), 5.76-5.69 (m, 1H), 5.07 (d, J = 10.40 Hz, 1H), 4.98- 4.85 (m, 2H), 4.62 (d, J = 4.40 Hz, 2H), 3.93 (s, 3H), 2.85-2.84 (m, 1H), 2.14- 2.11 (m, 4H), 2.09-2.00 (m, 3H), 1.61- 1.57 (m, 1H), 1.37-1.24 (m, 1H), 1.02 (d, J = 6.00 Hz, 3H). |
| 383 | 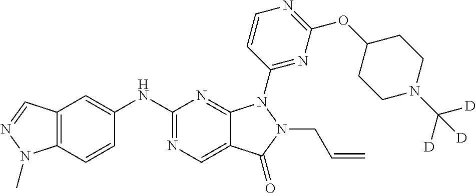 [SMILES: [2H]C([2H])([2H])N1CCC(Oc2nccc(-n3c4nc(Nc5ccc6c(cnn6C)c5)ncc4c(=O)n3CC=C)n2)CC1] | 516.3 | 1H-NMR (400 MHz, DMSO-d6): δ10.57 (s, 1H), 8.93 (s, 1H), 8.66 (d, 1H), 8.14 (s, 1H), 8.09 (s, 1H), 7.66 (s, 2H), 5.74-5.67 (m, 1H), 5.07 (d, 1H), 5.01-4.97 (m, 2H), 4.76 (d, 2H), 4.06 (s, 1H), 2.68-2.63 (m, 2H), 2.18 (t, 2H), 2.01-1.99 (m, 2H), 1.78-1.70 (m, 2H). |
| 384 | 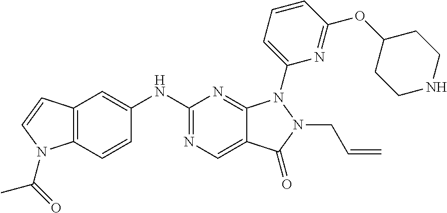 [SMILES: C=CCn1c(=O)c2cnc(Nc3ccc4c(ccn4C(C)=O)c3)nc2n1-c1cccc(OC2CCNCC2)n1] | 525.2 | 1H-NMR (400 MHz, DMSO-d6): δ 10.37 (s, 1H), 8.89 (s, 1H), 8.24 (d, J = 8.80 Hz, 1H), 8.12 (s, 1H), 7.95 (t, J = 8.00 Hz, 1H), 7.85 (d, J = 3.60 Hz, 1H), 7.57 (dd, J = 1.60, 9.20 Hz, 1H), 7.48 (d, J = 7.60 Hz, 1H), 6.81 (d, J = 8.00 Hz, 1H), 6.72 (d, J = 3.60 Hz, 1H), 5.74- 5.67 (m, 1H), 5.08-4.92 (m, 3H), 4.62 (d, J = 5.60 Hz, 2H), 3.00-2.97 (m, 2H), 2.68-2.65 (m, 5H), 1.96-1.91 (m, 2H), 1.60-1.51 (m, 2H). |
| 385 | 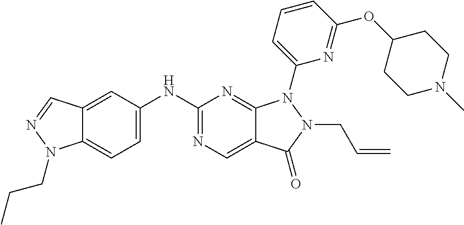 [SMILES: C=CCn1c(=O)c2cnc(Nc3ccc4c(cnn4CCC)c3)nc2n1-c1cccc(OC2CCN(C)CC2)n1] | 540.3 | 1H-NMR (400 MHz, DMSO-d6): δ 10.35 (s, 1H), 0.84 (s, 1H), 8.23 (s, 1H), 8.03 (s, 1H), 7.98 (t, 1H), 7.65-7.58 (m, 3H), 7.49 (d, 1H), 6.79 (d, 1H), 5.76- 5.67 (m, 1H), 5.06 (d, 1H), 4.95 (m, 2H), 4.62 (s, 2H), 4.35 (t, 2H), 2.59 (m, 2H), 2.15 (m, 5H). 1.94 (m, 2H), 1.82 (m, 2H), 1.71-1.66 (m, 2H), 0.83 (t, 3H). |
| 386 | 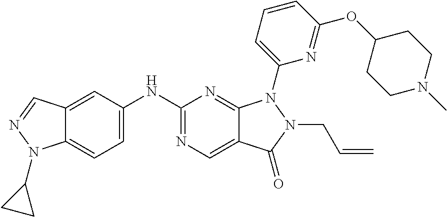 [SMILES: C=CCn1c(=O)c2cnc(Nc3ccc4c(cnn4C4CC4)c3)nc2n1-c1cccc(OC2CCN(C)CC2)n1] | 538.2 | 1H-NMR (400 MHz, DMSO-d6): δ 10.37 (br s, 1H), 8.88 (s, 1H), 8.24 (br s, 1H), 7.98 (t, J = 9.20 Hz, 2H), 7.65 (s, 2H), 7.48 (d, J = 7.60 Hz, 1H), 6.79 (d, J = 8.00 Hz, 1H), 5.75-5.69 (m, 1H), 5.06 (d, J = 9.60 Hz, 1H), 4.96-4.92 (m, 2H), 4.61 (br s, 2H), 3.75-3.72 (m, 1H), 2.68- 2.51 (m, 2H), 2.17-2.14 (m, 5H), 1.93- 1.87 (m, 2H), 1.76-1.69 (m, 2H), 1.11- 1.10 (m, 4H) |
| 387 | 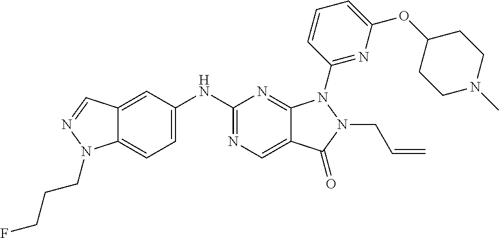 [SMILES: C=CCn1c(=O)c2cnc(Nc3ccc4c(cnn4CCCF)c3)nc2n1-c1cccc(OC2CCN(C)CC2)n1] | 558.5 | 1H-NMR (400 MHz, DMSO-d6): δ 10.37 (s, 1H), 8.88 (s, 1H), 8.25 (s, 1H), 8.06 (s, 1H), 7.99 (t, J = 7.60 Hz, 1H), 7.62 (s, 2H), 7.49 (d, J = 7.60 Hz, 1H), 6.80 (d, J = 8.00 Hz, 1H), 5.76-5.69 (m, 1H), 5.06 (d, J = 9.60 Hz, 1H), 4.96- 4.92 (m, 2H), 4.62 (d, J = 4.40 Hz, 2H), 4.50-4.47 (m, 3H), 4.37 (t, J = 5.60 Hz, 1H), 2.68-2.59 (m, 2H), 2.34-2.19 (m, 7H), 1.95-1.93 (m, 2H), 1.71-1.67 (m, 2H). |
| 388 | 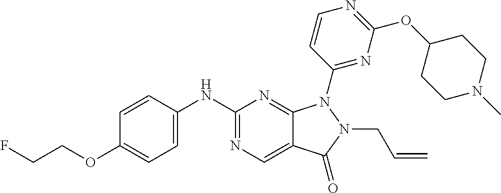 [SMILES: C=CCn1c(=O)c2cnc(Nc3ccc(OCCF)cc3)nc2n1-c1ccnc(OC2CCN(C)CC2)n1] | 519.2 | 1H-NMR (400 MHz, DMSO-d6): δ 10.41 (br s, 1H), 8.90 (s, 1H), 8.66 (d, J = 5.60 Hz, 1H), 7.71-7.62 (m, 3H), 7.04 (d, J = 6.80 Hz, 2H), 5.73-5.66 (m, 1H), 5.08-4.97 (m, 3H), 4.83-4.69 (m, 4H), 4.30-4.22 (m, 2H), 2.68-2.63 (m, 2H), 2.34-2.33 (m, 5H), 2.19-2.01 (m, 2H), 1.78-1.69 (m, 2H) |
| 389 | 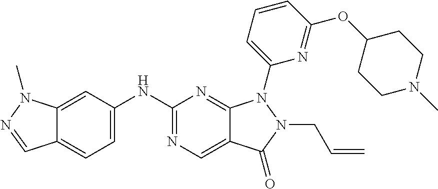 [SMILES: C=CCn1c(=O)c2cnc(Nc3ccc4cnn(C)c4c3)nc2n1-c1cccc(OC2CCN(C)CC2)n1] | 512.2 | 1H-NMR (400 MHz, DMSO-d6): δ 10.52 (br s, 1H), 8.95 (s, 1H), 8.20-8.16 (m, 1H), 7.99-7.94 (m, 2H), 7.66 (d, J = 8.80 Hz, 1H), 7.53 (d, J = 7.60 Hz, 1H), 7.29 (d, J = 8.40 Hz, 1H), 6.83 (d, J = 8.00 Hz, 1H), 5.76-5.68 (m, 1H), 5.07 (d, J = 9.60 Hz, 1H), 4.96-4.92 (m, 2H), 4.59 (d, J = 5.60 Hz, 2H), 3.94 (s, 3H), 2.68-2.62 (m, 2H), 2.19 (s, 5H), 1.92- 1.71 (m, 2H), 1.69-1.67 (m, 2H) |
| 390 | 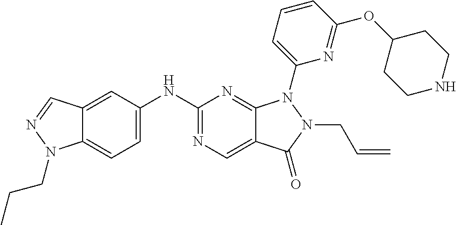 [SMILES: C=CCn1c(=O)c2cnc(Nc3ccc4c(cnn4CCC)c3)nc2n1-c1cccc(OC2CCNCC2)n1] | 526.3 | 1H-NMR (400 MHz, DMSO-d6): δ 10.3 (1H, s), 8.83 (s, 1H), 8.32 (s, 1H), 8.24 (s, 1H), 8.03 (s, 1H), 7.99 (m, 1H), 7.65- 7.58 (m, 2H), 7.50 (d, 1H), 6.80 (d, 1H), 5.71 (m, 1H), 5.08-7.92 (m, 3H), 4.63 (m, 2H), 4.35 (t, 2H), 3.02 (m, 2H), 2.74-2.67 (m, 2H), 1.97 (m, 2H), 1.85 (m, 2H), 1.60 (m, 2H), 0.83 (t, 3H). |
| 391 | 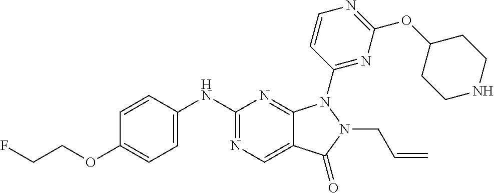 [SMILES: C=CCn1c(=O)c2cnc(Nc3ccc(OCCF)cc3)nc2n1-c1ccnc(OC2CCNCC2)n1] | 519.2 | 1H-NMR (400 MHz, DMSO-d6): δ 10.41 (br s, 1H), 8.90 (s, 1H), 8.66 (d, J = 5.60 Hz, 1H), 7.71-7.62 (m, 3H), 7.04 (d, J = 6.80 Hz, 2H), 5.73-5.66 (m, 1H), 5.08-4.97 (m, 3H), 4.83-4.69 (m, 4H), 4.30-4.22 (m, 2H), 2.68-2.63 (m, 2H), 2.34-2.33 (m, 5H), 2.19-2.01 (m, 2H), 1.78-1.69 (m, 2H) |
| 392 | 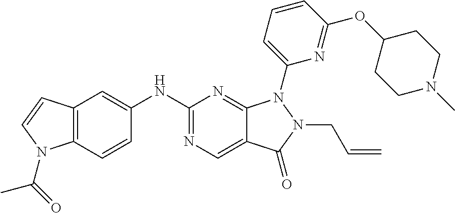 [SMILES: C=CCn1c(=O)c2cnc(Nc3ccc4c(ccn4C(C)=O)c3)nc2n1-c1cccc(OC2CCN(C)CC2)n1] | 539.3 | 1H-NMR (400 MHz, DMSO-d6): δ 10.37 (s, 1H), 8.89 (s, 1H), 8.23 (d, J = 8.80 Hz, 1H), 8.12 (s, 1H), 7.94 (t, J = 8.00 Hz, 1H), 7.85 (d, J = 3.60 Hz, 1H), 7.57 (d, J = 8.80 Hz, 1H), 7.48 (d, J = 7.60 Hz, 1H), 6.80 (d, J = 8.40 Hz, 1H), 6.72 (d, J = 3.60 Hz, 1H), 5.74-5.67 (m, 1H), 5.08-5.05 (m, 1H), 4.96-4.92 (m, 2H), 4.62-4.61 (m, 2H), 2.65-2.60 (m, 5H), 2.17-2.14 (m, 5H), 2.01-1.93 (m, 2H), 1.69-1.67 (m, 2H). |
| 393 | 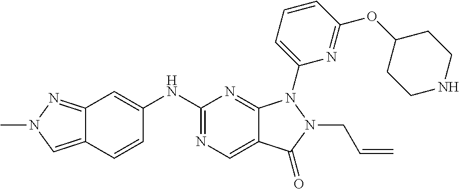 [SMILES: C=CCn1c(=O)c2cnc(Nc3ccc4cn(C)nc4c3)nc2n1-c1cccc(OC2CCNCC2)n1] | 498.2 | 1H-NMR (400 MHz, DMSO-d6,): δ 10.52 (s, 1H), 8.95 (s, 1H), 8.32 (s, 1H), 8.20 (s, 1H), 8.00-7.94 (m, 2H), 7.66 (dd, J = 0.40, 8.80 Hz, 1H), 7.54 (d, J = 7.20 Hz, 1H), 7.29 (dd, J = 1.60, 8.80 Hz, 1H), 6.84 (d, J = 7.60 Hz, 1H), 5.76- 5.69 (m, 1H), 5.08-5.02 (m, 2H), 4.94 (dd, J = 1.20, 17.00 Hz, 1H), 4.60 (d, J = 6.00 Hz, 2H), 3.94 (s, 3H), 3.03-2.99 (m, 2H), 2.71-2.67 (m, 2H), 1.97-1.94 (m, 2H), 1.61-1.58 (m, 2H). |
| 394 | 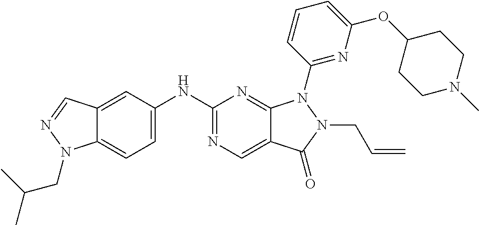 [SMILES: C=CCn1c(=O)c2cnc(Nc3ccc4c(cnn4CC(C)C)c3)nc2n1-c1cccc(OC2CCN(C)CC2)n1] | 554.30 | 1H-NMR (400 MHz, DMSO-d6): δ 10.35 (s, 1H), 8.88 (s, 1H), 8.23 (s, 1H), 8.19 (s, 1H), 8.03 (s, 1H), 7.98 (t, J = 8.00 Hz, 1H), 7.65-7.58 (m, 2H), 7.49 (d, J = 7.60 Hz, 1H), 6.80 (d, J = 8.40 Hz, 1H), 5.73-5.67 (m, 1H), 5.06 (dd, J = 0.80, 10.40 Hz, 1H), 4.95 (dd, J = 4.40, 8.20 Hz, 1H), 4.93-4.92 (m, 1H), 4.63-4.62 (m, 2H), 4.20 (d, J = 7.20 Hz, 2H), 2.68-2.61 (m, 2H), 2.26-2.19 (m, 6H), 1.96-1.94 (m, 2H), 1.72-1.64 (m, 2H), 0.86 (d, J = 6.80 Hz, 6H). |
| 395 | 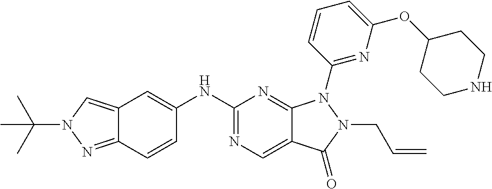 [SMILES: C=CCn1c(=O)c2cnc(Nc3ccc4nn(C(C)(C)C)cc4c3)nc2n1-c1cccc(OC2CCNCC2)n1] | 540.6 | 1H-NMR (400 MHz, DMSO-d6): δ 10.29 (s, 1H), 8.88 (s, 1H), 8.35 (d, J = 16.40 Hz, 2H), 8.15 (s, 1H), 8.02 (s, 1H), 7.59 (d, J = 9.20 Hz, 1H), 7.52- 7.45 (m, 2H), 6.80 (d, J = 8.40 Hz, 1H), 5.77-5.67 (m, 1H), 5.08-4.93 (m, 3H), 4.63 (d, J = 4.40 Hz, 2H), 3.05-2.99 (m, 2H), 2.73-2.67 (m, 2H), 1.98-1.96 (m, 2H), 1.69 (s, 9H), 1.62-1.61 (m, 2H). |
| 396 | 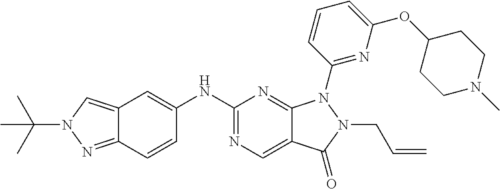 [SMILES: C=CCn1c(=O)c2cnc(Nc3ccc4nn(C(C)(C)C)cc4c3)nc2n1-c1cccc(OC2CCN(C)CC2)n1] | 554.48 | 1H-NMR (400 MHz, DMSO-d6): δ 10.29 (s, 1H), 8.88 (s, 1H), 8.37 (s, 1H), 8.15 (s, 1H), 8.01 (s, 1H), 7.58 (d, J = 9.20 Hz, 1H), 7.51-7.44 (m, 2H), 6.79 (d, J = 8.00 Hz, 1H), 5.74-5.69 (m, 1H), 5.06 (dd, J = 0.80, 10.20 Hz, 1H), 4.97- 4.92 (m, 2H), 4.62 (d, J = 4.40 Hz, 2H), 2.62-2.59 (m, 2H), 2.17-2.14 (m, 5H), 1.95-1.93 (m, 2H), 1.70-1.68 (m, 11H). |
| 397 | 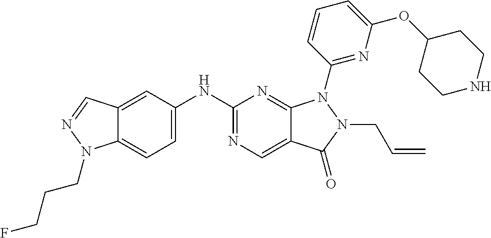 [SMILES: C=CCn1c(=O)c2cnc(Nc3ccc4c(cnn4CCCF)c3)nc2n1-c1cccc(OC2CCNCC2)n1] | 544.5 | 1H-NMR (400 MHz, DMSO-d6,): δ 10.39 (s, 1H), 8.88 (s, 1H), 8.25 (s, 1H), 8.06 (s, 1H), 7.98 (t, J = 8.00 Hz, 1H), 7.62 (s, 2H), 7.49 (d, J = 7.60 Hz, 1H), 6.79 (d, J = 8.40 Hz, 1H), 5.73-5.69 (m, 1H), 5.06 (dd, J = 1.20, 10.20 Hz, 1H), 4.98-4.92 (m, 2H), 4.63 (d, J = 4.40 Hz, 2H), 4.52-4.49 (m, 3H), 4.37 (t, J = 5.60 Hz, 1H), 2.97-2.92 (m, 2H), 2.58-2.55 (m, 2H), 2.26-2.16 (m, 2H), 1.93-1.90 (m, 2H), 1.51-1.48 (m, 2H). |
| 398 | 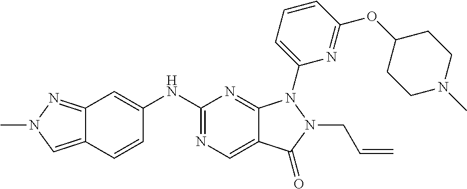 | 512.3 | 1H-NMR (400 MHz, DMSO-d6): δ 10.51 (s, 1H), 8.95 (s, 1H), 8.20 (s, 1H), 7.99-7.94 (m, 2H), 7.66 (d, J = 8.80 Hz, 1H), 7.53 (d, J = 7.60 Hz, 1H), 7.29 (d, J = 8.40 Hz, 1H), 6.83 (d, J = 8.00 Hz, 1H), 5.76-5.69 (m, 1H), 5.07 (dd, J = 1.20, 10.40 Hz, 1H), 4.96-4.91 (m, 2H), 4.59 (d, J = 6.00 Hz, 2H), 3.94 (s, 3H), 2.60-2.51 (m, 2H), 2.20-2.12 (m, 5H), 1.94-1.92 (m, 2H), 1.71-1.62 (m, 2H). |
| 399 | 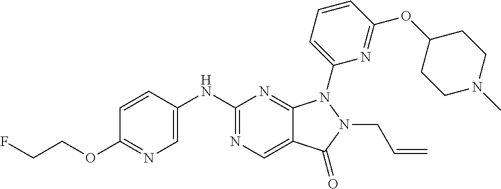 [SMILES: C=CCn1c(=O)c2cnc(Nc3ccc(OCCF)nc3)nc2n1-c1cccc(OC2CCN(C)CC2)n1] | 521.2 | 1H-NMR (400 MHz, DMSO-d6): δ 10.27 (s, 1H), 8.88 (s, 1H), 8.40 (d, J = 2.40 Hz, 1H), 8.06 (d, J = 7.60 Hz, 1H), 7.91 (t, J = 8.00 Hz, 1H), 7.38-7.37 (m, 1H), 6.89 (d, J = 8.80 Hz, 1H), 6.79 (d, J = 8.00 Hz, 1H), 5.74-5.67 (m, 1H), 5.06 (d, J = 9.60 Hz, 1H), 4.95-4.91 (m, 2H), 4.82-4.80 (m, 1H), 4.75 (d, J = 40.00 Hz, 1H), 4.69-4.58 (m, 2H), 4.53-4.51 (m, 1H), 4.45-4.43 (m, 1H), 2.61-2.59 (m, 2H), 2.17-2.14 (m, 5H), 1.96-1.93 (m, 2H), 1.70-1.66 (m, 2H). |
| 400 | 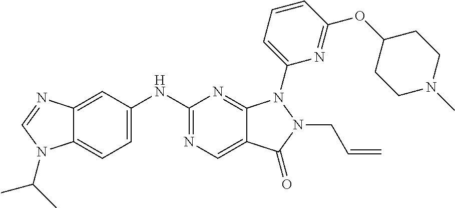 [SMILES: C=CCn1c(=O)c2cnc(Nc3ccc4c(c3)ncn4C(C)C)nc2n1-c1cccc(OC2CCN(C)CC2)n1] | 540.2 | 1H-NMR (400 MHz, DMSO-d6): δ 10.33 (s, 1H), 8.88 (s, 1H), 8.29 (s, 1H), 8.14 (s, 1H), 7.92 (t, J = 8.00 Hz, 1H), 7.55 (dd, J = 7.60, 18.00 Hz, 3H), 6.80 (d, J = 8.00 Hz, 1H), 5.75-5.67 (m, 1H), 5.01 (dd, J = 1.20, 37.00 Hz, 1H), 4.94 (dd, J = 1.60, 8.00 Hz, 2H), 4.77-4.71 (m, 1H), 4.70-4.62 (m, 2H), 2.53-2.52 (m, 2H), 2.17 (s, 5H), 1.97-1.94 (m, 2H), 1.69 (s, 2H), 1.54 (d, J = 6.80 Hz, 6H). |
| 401 | 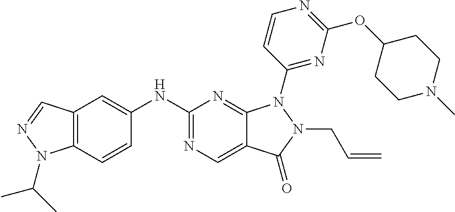 [SMILES: C=CCn1c(=O)c2cnc(Nc3ccc4c(cnn4C(C)C)c3)nc2n1-c1ccnc(OC2CCN(C)CC2)n1] | 541.3 | 1H-NMR (400 MHz, DMSO-d6): δ 10.57 (br s, 1H), 8.93 (s, 1H), 8.66 (d, J = 5.60 Hz, 1H), 8.12 (d, J = 13.20 Hz, 2H), 7.74-7.62 (m, 3H), 5.75-5.65 (m, 1H), 5.08-4.97 (m, 4H), 4.76-4.75 (m, 2H), 2.68-2.63 (m, 2H), 2.19-2.16 (m, 5H), 1.99 (s, 2H), 1.77-1.70 (m, 2H), 1.51-1.45 (m, 6H) |
| 402 | 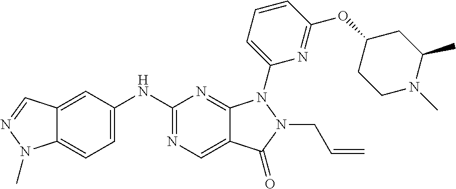 [SMILES: C=CCn1c(=O)c2cnc(Nc3ccc4c(cnn4C)c3)nc2n1-c1cccc(O[C@H]2CCN(C)[C@H](C)C2)n1] | 526.3 | 1H-NMR (400 MHz, DMSO-d6):): δ 10.05 (s, 1H), 8.83 (s, 1H), 8.17 (d, J = 1.20 Hz, 1H), 7.96-7.92 (m, 2H), 7.64 (dd, J = 1.60, 9.00 Hz, 1H), 7.55 (d, J = 9.20 Hz, 2H), 7.46 (d, J = 7.60 Hz, 1H), 6.79 (d, J = 8.40 Hz, 1H), 5.74-5.70 (m, 1H), 5.19 (t, J = 3.60 Hz, 1H), 5.07 (dd, J = 1.20, 10.40 Hz, 1H), 4.98 (dd, J = 1.20, 17.20 Hz, 1H), 4.60 (d, J = 5.60 Hz, 2H), 4.04 (s, 3H), 2.59-2.55 (m, 1H), 2.41-2.33 (m, 2H), 2.20 (s, 3H), 1.82-1.78 (m, 9H), 1.60-1.56 (m, 1H), |
| | | | 0.98 (d, J = 6.40 Hz, 3H), (Acetic acid |
| | | | salt). |
| 403 | 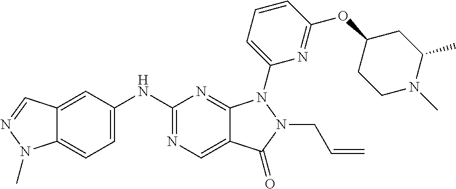 [SMILES: C=CCn1c(=O)c2cnc(Nc3ccc4c(cnn4C)c3)nc2n1-c1cccc(O[C@@H]2CCN(C)[C@@H](C)C2)n1] | 526.3 | 1H-NMR (400 MHz, DMSO-d6): δ 10.35 (s, 1H), 8.88 (s, 1H), 8.24 (s, 1H), 8.02-7.97 (m, 2H), 7.60-7.58 (m, 2H), 7.48 (d, J = 8.00 Hz, 1H), 7.48 (d, J = 8.00 Hz, 1H), 5.74-5.68 (m, 1H), 5.13- 5.11 (m, 1H), 5.06 (d, J = 9.60 Hz, 1H), 4.93 (d, J = 17.20 Hz, 1H), 4.61 (d, J = 4.00 Hz, 2H), 4.04 (s, 3H), 2.36-2.32 (m, 2H), 2.18 (s, 3H), 1.84-1.76 (m, 11H), 1.57-1.51 (m, 1H), 0.97 (d, J = 6.00 Hz, 3H), (Acetic acid salt). |
| 404 | 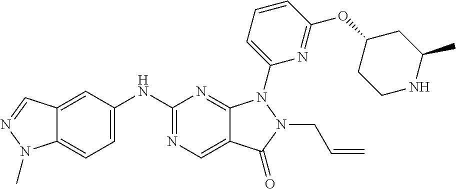 [SMILES: C=CCn1c(=O)c2cnc(Nc3ccc4c(cnn4C)c3)nc2n1-c1cccc(O[C@H]2CCN[C@H](C)C2)n1] | 512.3 | 1H-NMR (400 MHz, DMSO-d6): δ 10.42 (s, 1H), 9.36-9.34 (m, 1H), 9.00- 8.97 (m, 1H), 8.89 (s, 1H), 8.25-8.24 (m, 1H), 8.07-8.03 (m, 2H), 7.64-7.59 (m, 2H), 7.53 (d, J = 7.60 Hz, 1H), 6.87 (d, J = 8.00 Hz, 1H), 5.74-5.68 (m, 1H), 5.34 (m, 1H), 5.06 (dd, J = 1.20, 10.40 Hz, 1H), 4.95 (dd, J = 1.20, 17.20 Hz, 1H), 4.64-4.63 (m, 2H), 4.03 (s, 3H), 3.49-3.39 (m, 2H), 3.17-3.16 (m, 1H), 3.10-2.99 (m, 1H), 2.09-2.04 (m, 3H), 1.88-1.85 (m, 1H), 1.25 (d, J = 8.00 Hz, |
| | | | 2H). (HCl salt). |
| 405 | 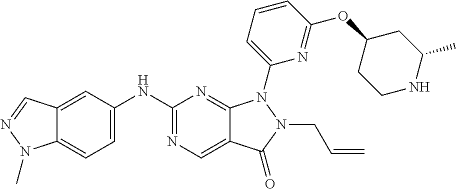 [SMILES: C=CCn1c(=O)c2cnc(Nc3ccc4c(cnn4C)c3)nc2n1-c1cccc(O[C@@H]2CCN[C@@H](C)C2)n1] | 512.3 | 1H-NMR (400 MHz, DMSO-d6): δ 10.52 (br s, 1H), 9.35 (s, 1H), 8.91 (s, 1H), 8.89 (s, 1H), 8.24-8.16 (m, 1H), 8.07-8.03 (m, 2H), 7.64-7.59 (m, 2H), 7.53 (d, J = 7.60 Hz, 1H), 6.94 (d, J = 40.00 Hz, 1H), 5.74-5.66 (m, 1H), 5.34- 5.34 (m, 1H), 5.06 (dd, J = 1.20, 10.40 Hz, 1H), 4.95 (dd, J = 1.20, 17.20 Hz, 1H), 4.63-4.62 (m, 2H), 4.03 (s, 3H), 3.40-3.39 (m, 2H), 3.17-3.16 (m, 1H), 3.11-3.03 (m, 1H), 2.09-2.04 (m, 3H), 1.42-1.31 (m, 1H), 1.25 (d, J = 6.40 Hz, |
| | | | 3H), (HCl salt). |
| 406 | 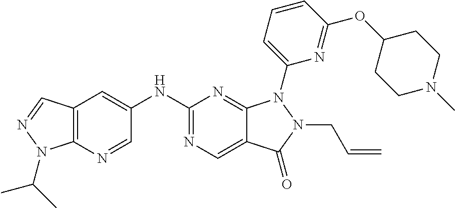 [SMILES: C=CCn1c(=O)c2cnc(Nc3cnc4c(cnn4C(C)C)c3)nc2n1-c1cccc(OC2CCN(C)CC2)n1] | 541.3 | 1H-NMR (400 MHz, DMSO-d6, at 80 °C): δ 10.19 (s, 1H), 8.87 (s, 1H), 8.72 (d, J = 2.40 Hz, 1H), 8.53 (d, J = 2.40 Hz, 1H), 8.08 (s, 1H), 7.90 (t, J = 7.60 Hz, 1H), 7.41 (d, J = 7.60 Hz, 1H), 6.79 (d, J = 8.40 Hz, 1H), 5.78-5.71 (m, 1H), 5.20-5.17 (m, 1H), 5.07-4.95 (m, 3H), 4.60 (d, J = 6.00 Hz, 2H), 2.64-2.58 (m, 2H), 2.22-2.16 (m, 5H), 1.97-1.92 (m, 2H), 1.74-1.68 (m, 2H), 1.54 (d, J = 6.80 Hz, 6H). |
| 407 | 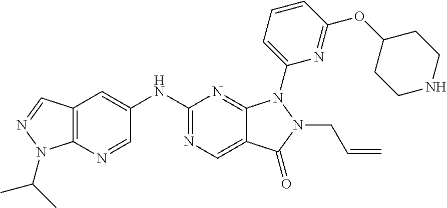 [SMILES: C=CCn1c(=O)c2cnc(Nc3cnc4c(cnn4C(C)C)c3)nc2n1-c1cccc(OC2CCNCC2)n1] | 527.3 | 1H-NMR (400 MHz, DMSO-d6, at 80 °C): δ 10.18 (s, 1H), 8.86(s, 1H), 8.72 (d, J = 2.40 Hz, 1H), 8.53 (d, J = 2.40 Hz, 1H), 8.24 (s, 1H), 8.08 (s, 1H), 7.90 (t, J = 7.60 Hz, H), 7.42 (d, J = 7.60 Hz, 1H), 6.79 (d, J = 8.00 Hz, 1H), 5.78- 5.71 (m, 1H), 5.17 (dd, J = 6.80, 13.20 Hz, 1H), 5.07-4.97 (m, 3H), 4.60 (d, J = 6.00 Hz, 2H), 3.03-2.98 (m, 2H), 2.69- 2.62 (m, 2H), 1.97-1.94 (m, 2H), 1.63- 1.55 (m, 8H). |
| 408 | 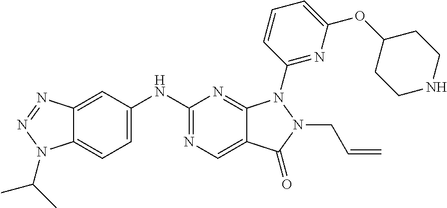 [SMILES: C=CCn1c(=O)c2cnc(Nc3ccc4c(c3)nnn4C(C)C)nc2n1-c1cccc(OC2CCNCC2)n1] | 527.2 | 1H-NMR (400 MHz, DMSO-d6,): δ 10.50 (s, 1H), 8.95 (s, 1H), 8.33 (s, 1H), 7.96 (t, J = 8.00 Hz, 1H), 7.88 (d, J = 8.80 Hz, 1H), 7.75 (dd, J = 2.00, 9.00 Hz, 1H), 7.51 (d, J = 7.60 Hz, 1H), 6.85 (d, J = 8.40 Hz, 1H), 5.77-5.70 (m, 1H), 5.21-5.16 (m, 1H), 5.09-5.06 (m, 2H), 4.98 (dd, J = 16.00, 22.60 Hz, 1H), 4.63 (d, J = 5.60 Hz, 2H), 3.06-3.00 (m, 2H), 2.73-2.67 (m, 2H), 2.00-1.99 (m, 2H), 1.65-1.59 (m, 8H) |
| 409 | 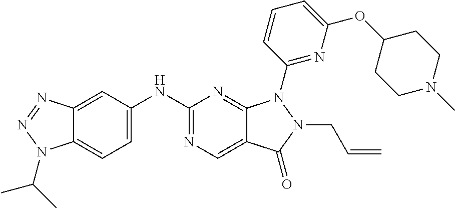 [SMILES: C=CCn1c(=O)c2cnc(Nc3ccc4c(c3)nnn4C(C)C)nc2n1-c1cccc(OC2CCN(C)CC2)n1] | 541.2 | 1H-NMR (400 MHz, DMSO-d6): δ 10.52 (s, 1H), 8.95 (s, 1H), 8.57-8.56 (m, 1H), 8.30 (s, 1H), 7.99-7.93 (m, 1H), 7.88 (d, J = 8.80 Hz, 1H), 7.75 (s, 1H), 7.52-7.49 (m, 1H), 6.85 (t, J = 8.80 Hz, 1H), 5.76-5.68 (m, 1H), 5.20 (q, J = 6.80 Hz, 2H), 5.16-5.06 (m, 1H), 4.95 (d, J = 8.40 Hz, 1H), 4.63 (d, J = 4.80 Hz, 2H), 3.08-3.05 (m, 2H), 2.79 (t, J = 9.60 Hz, 1H), 2.68-2.61 (m, 1H), 2.17- 2.14 (m, 2H), 2.02-2.00 (m, 2H), 1.70- 1.63 (m, 8H). |

or a pharmaceutically acceptable salt thereof.

### 4. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that is effective to measurably inhibit Wee1A kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some embodiments, the compounds or compositions disclosed herein are administered orally. Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration.

The amount of compounds of the present disclosure that may be combined with the carrier materials to produce a composition in a single dosage form will vary depending upon the host treated, the particular mode of administration. Preferably, provided compositions should be formulated so that a dosage of between 0.001-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the inhibition of protein kinase activity of one or more enzymes.

Examples of kinases that are inhibited by the compounds and compositions described herein and against which the methods described herein are useful include Wee1A kinase, or both Wee1A kinase and Myt1 kinase.

The activity of a compound utilized in this disclosure as an inhibitor of Wee1A kinase or Myt1 kinase, or a mutant of either of the foregoing, may be assayed in vitro, in vivo or in a cell line. In vitro assays include assays that determine inhibition of either the phosphorylation activity and/or the subsequent functional consequences, or ATPase activity of activated Wee1A kinase, activated Myt1 kinase, or a mutant of either of the foregoing. Alternate in vitro assays quantitate the ability of the inhibitor to bind to Wee1A kinase.

The inhibition of the DNA damage response (DDR) pathway in the treatment of cancer has recently gained interest, and different DDR inhibitors have been developed. Among them, the most promising ones target the Wee1 kinase family, which has a crucial role in cell cycle regulation and DNA damage identification and repair in both nonmalignant and cancer cells.

### Wee1 Kinase Family

The Wee1 kinase family consists of three serine/threonine kinases sharing conserved molecular structures and encoded by the following genes: WEE1 (alternatively, WEE1 G2 checkpoint kinase or Wee1A kinase), PKMYT1 (alternatively MYT1 kinase or membrane-associated tyrosine- and threonine-specific cdc2-inhibitory kinase), and WEE2 (alternatively WEE2 oocyte meiosis inhibiting kinase or Wee1B kinase). In eukaryotic somatic cells, Wee1A kinase and Myt1 kinase play a key role in cell cycle regulation, in particular, in the entry into mitosis (Schmidt M, Rohe A, Platzer C, et al. Regulation of G2/M transition by inhibition of Wee1 and PMyt1 Kinases. Molecules. 2017; 22:2045). Their role as regulators is crucial during normal cell cycle progression and in response to DNA damage as part of the DNA damage response (DDR) pathways. Similarly, Wee1B kinase regulates cell cycle progression and, in particular, meiosis (Sole P, Schultz R M, Motlik J. Prophase I arrest and progression to metaphase I in mouse oocytes: Comparison of resumption of meiosis and recovery from G2-arrest in somatic cells. Mol Hum Reprod. 2010; 16:654-64).

### Wee1B Kinase

Wee1B kinase expression is germ-cell specific and inhibits meiosis by phosphorylating Tyr15 of the CDK1-cyclin B complex (JY Zhu et al., J Med Chem. 2017; 60 (18), 7863-7875). Previous and current drug discovery efforts have not been focused on Wee1B kinase due to its characterized role in meiosis. Wee1B kinase plays a dual regulatory role in oocyte meiosis by preventing premature restart prior to ovulation and permitting metaphase II exit at fertilization (Nakanishi M, Ando H, Watanabe N, et al. Identification and characterization of human Wee1B, a new member of the Wee1 family of Cdk-inhibitory kinases. Genes Cells. 2000: 5 (10): 839-47). Despite the identification of WEE2 somatic mutations (1.9% of cases) and copy number (CN) alterations (22.5% of patients with CN loss and 22.5% with CN gain) across several cancer types (https://portal.gdc.cancer.gov), they have not yet been functionally linked to tumor development.

### Myt1 Kinase

Myt1 kinase is a multi-functional protein kinase localized to the ER-Golgi complex that is known to play a regulatory role in the cell cycle by inhibiting Cdk1/cyclin B1 mediated mitosis (JY Zhu et al., J Med Chem. 2017; 60 (18), 7863-7875). As mentioned above and throughout, Myt1 kinase inhibits the Cdk1/cyclin B1 activity through the phosphorylation of Tyr15 and Thr14 of Cdk1 and sequestration of Cdk1 from the nucleus. Additionally, Myt1 kinase has been tied to orchestrating the ER-Golgi complex reassembly during mitotic exit.

### Wee1A Kinase

Wee1A kinase regulates entry into mitosis at the G2/M transition of the S phase by phosphorylating Tyr15 of Cdk1 to inactive the Cdk1/cyclin B complex. Cells with perturbed G1 checkpoint activity (e.g., cancer cells) rely on Wee1A kinase to inhibit Cdk1 to permit a G2/M arrest for DNA repair. If Wee1A kinase activity is altered, a perturbed cell may enter mitosis prematurely without having the opportunity to fully replicate the entire DNA content or repair potential DNA lesions that might have occurred during S phase. This characterization of Wee1A kinase's role in the cell cycle has made it an attractive target for anticancer therapeutics, especially in combination with DNA-damaging agents (J Y Zhu et al., J Med Chem. 2017; 60 (18), 7863-7875).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of Wee1A kinase and are therefore useful for treating one or more disorders associated with activity of Wee1A kinase. Thus, in certain embodiments, the present disclosure provides a method for treating a Wee1A kinase-mediated disorder comprising the step of administering to a patient in need thereof a compound of the present disclosure, or pharmaceutically acceptable composition thereof.

Some of the provided compounds also demonstrate potent inhibitory activity against Myt1 kinase and therefore are dual inhibitors useful for treating one or more disorders associated with activity of both Wee1A kinase and Myt1 kinase. Thus, in certain embodiments, the present disclosure provides a method for treating a Wee1A kinase/Myt1 kinase-mediated disorder comprising the step of administering to a patient in need thereof a compound of the present disclosure that is a dual inhibitor, or a pharmaceutically acceptable composition thereof.

As used herein, the term “Wee1A kinase-mediated” disorder or condition as used herein means any disease or other deleterious condition in which Wee1A kinase, or a mutant thereof, is known to play a role. Accordingly, another embodiment of the present disclosure relates to treating or lessening the severity of one or more diseases in which Wee1A kinase, or a mutant thereof, is known to play a role. Specifically, the present disclosure relates to a method of treating or lessening the severity of a disease or condition selected from a proliferative disorder, wherein said method comprises administering to a patient in need thereof a compound or composition according to the present disclosure.

As used herein, the term “Wee1A kinase/Myt1 kinase-mediated” disorder or condition as used herein means any disease or other deleterious condition in which both Wee1A kinase and Myt1 kinase, or a mutant of either or both of the foregoing, are known to play a role. Accordingly, another embodiment of the present disclosure relates to treating or lessening the severity of one or more diseases in which both Wee1A kinase and Myt1 kinase, or a mutant of either or both of the foregoing, are known to play a role. Specifically, the present disclosure relates to a method of treating or lessening the severity of a disease or condition selected from a proliferative disorder, wherein said method comprises administering to a patient in need thereof a compound or composition according to the present disclosure.

In some embodiments, the present disclosure provides a method of inhibiting Wee1A kinase activity in a subject comprising the step of administering to the subject an effective amount of a compound, or a pharmaceutically acceptable composition, of the present disclosure.

In some embodiments, the present disclosure provides a method of inhibiting both Wee1A kinase and Myt1 kinase activity in a subject comprising the step of administering to the subject an effective amount of a compound of the present disclosure that is a dual inhibitor, or a pharmaceutically acceptable composition.

In some embodiments, the present disclosure provides a method for treating or lessening the severity of one or more disorders selected from a cancer comprising the step of administering to the subject an effective amount of a compound, or a pharmaceutically acceptable composition thereof, of the present disclosure. In some embodiments, the cancer is associated with a solid tumor.

In some embodiments, the present disclosure provides a method of treating a subject suffering from a cancer or other disordered cell growth characterized by aberrant Wee1A kinase activity comprising the step of administering to the subject an effective amount of a compound, or a pharmaceutically acceptable composition thereof, of the present disclosure. In some embodiments, aberrant Wee1A kinase activity includes elevated activity, or overexpression, or undesirable activity as compared to a non-diseased state. In some such embodiments, aberrant Wee1A kinase activity may include perturbed p53 activity, Cdk1 activity, Cdk2 activity, replication stress, altered mitosis, and DNA damage. In some embodiments, the subject is suffering from a cancer associated with inactivation of p53.

In some embodiments, the present disclosure provides a method of treating a subject suffering from a cancer or other disordered cell growth characterized by both aberrant Wee1A kinase and aberrant Myt1 kinase activity comprising the step of administering to the subject an effective amount of a compound of the present disclosure that is a dual inhibitor, or a pharmaceutically acceptable composition thereof. In some embodiments, aberrant Wee1A kinase and aberrant Myt1 kinase activity includes elevated activity, or overexpression, or undesirable activity as compared to a non-diseased state. In some such embodiments, aberrant Wee1A kinase activity and aberrant Myt1 kinase activity may include perturbed Cdk1 activity, replication stress, altered mitosis, and DNA damage. In some embodiments, the subject to be treated has previously been treated with either a mono-specific Wee1A kinase inhibitor or Myt1 kinase inhibitor (neither of which is a dual inhibitor) and has developed resistance to or is refractory to such treatment. For example, such a subject could be resistant or refractory to the Myt1 kinase inhibitor RP-6306, or the Wee1A kinase inhibitors AZD1775, Debio0123 or ZnC₃.

In some embodiments, the cancer to be treated by a compound disclosed herein is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some embodiments, the subject is suffering from a cancer selected from a uterine serous carcinoma and a renal cancer.

In some embodiments, the breast cancer is selected from ductal carcinoma in situ (DCIS), invasive ductal carcinoma (IDC), lobular carcinoma in situ (LCIS), invasive lobular cancer (ILC), triple negative breast cancer (TNBC), inflammatory breast cancer (IBC), metastatic breast cancer (MBC), medullary carcinoma, tubular carcinoma, mucinous carcinoma (colloid), and Paget disease of the breast or nipple (commonly known as Paget disease).

In some embodiments, the uterine cancer is selected from endometrial cancer and uterine sarcoma. In some embodiments, the uterine cancer is endometrial cancer. In some embodiments, the uterine cancer is uterine sarcoma.

In some embodiments, the ovarian cancer is selected from epithelial ovarian carcinomas, germ cell tumors, and stromal cell tumors.

In some embodiments, the stomach cancer is selected from adenocarcinoma, lymphoma, gastrointestinal stromal tumors (GISTs), carcinoid tumors, and hereditary (familial) diffuse gastric cancer.

In some embodiments the esophageal cancer is selected from squamous cell carcinoma, small cell carcinoma, and adenocarcinoma. In some embodiments the esophageal cancer is selected from squamous cell carcinoma and adenocarcinoma. In some embodiments, the esophageal cancer is squamous cell carcinoma. In some embodiments, the esophageal cancer is adenocarcinoma.

In some embodiments, the lung cancer is selected from non-small cell lung cancer, lung nodules, small cell lung cancer, and mesothelioma. In some embodiments, the lung cancer is non-small cell lung cancer.

In some embodiments the colorectal cancer is selected from adenocarcinoma, gastrointestinal stromal tumors (GIST), lymphoma, carcinoids, Turcot syndrome, Peutz-Jeghers syndrome (PJS), familial colorectal cancer (FCC), and juvenile polyposis coli.

In some embodiments, the cancer is associated with deregulation of cyclin E1. In some embodiments, the cancer associated with deregulation of cyclin E1 is ovarian cancer.

In some embodiments, the cancer is associated with deregulation of p53. In some embodiments, the cancer associated with deregulation of p53 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of p53 is selected from uterine serous carcinoma and a renal cancer.

In some embodiments, the cancer is associated with deregulation of Cdk1. In some embodiments, the cancer associated with deregulation of Cdk1 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of Cdk1 is selected from uterine serous carcinoma and a renal cancer.

In some embodiments, the cancer is associated with deregulation of Cdk2. In some embodiments, the cancer associated with deregulation of Cdk2 is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In some such embodiments, the cancer associated with deregulation of Cdk1 is selected from uterine serous carcinoma and a renal cancer.

Depending upon the particular condition, or disease, to be treated, additional therapeutic agents, which are normally administered to treat that condition, may also be present in the compositions of this disclosure. As used herein, additional therapeutic agents that are normally administered to treat a particular disease, or condition, are known as “appropriate for the disease, or condition, being treated.”

For example, compounds of the present disclosure, or a pharmaceutically acceptable composition thereof, are administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, camptothecin, cisplatin, metronidazole, and Gleevec™, among others. In other embodiments, a compound of the present disclosure is administered in combination with a biologic agent, such as Avastin or VECTIBIX.

In some embodiments, compounds of the present disclosure or a pharmaceutically acceptable composition thereof, are administered in combination with an agent selected from fasudil, sirolimus, imatinib, gefitinib, erlotinib, sorafenib, sunitinib, dasatinib, lapatinib, nilotinib, temsirolimus, everolimus, pazopanib, ruxolitinib, vandetanib, vemurafenib, crizotinib, icotinib, axitinib, tofacitinib, bosutinib, cabozantinib, ponatinib, regorafenib, afatinib, dabrafenib, trametinib, ibrutinib, nintedanib, idelalisib, ceritinib, apatinib rivoceranib, ripasudil, alectinib, cobimetinib, lenvatinib, palbociclib, radotinib, osimertinib, olmutinib, neratinib, ribociclib, copanlisib, abemaciclib, acalabrutinib, midostaurin, brigatinib, baricitinib, netarsudil, tivozanib, simotinib, fostamatinib, encorafenib, binimetinib, catequentinib, duvelisib, dacomitinib, lorlatinib, larotrectinib, gilteritinib, pyrotinib, fruquintinib, erdafitinib, alpelisib, umbralisib, leniolisib, pexidartinib, entrectinib, upadacitinib, fedratinib, zanubrutinib, flumatinib, peficitinib, delgocitinib, avapritinib, selumetinib, tucatinib, pemigatinib, capmatinib tabrecta, selpercatinib, ripretinib, tirabrutinib, almonertinib, pralsetinib, filgotinib, tirbanibulin, orelabrutinib, tepotinib, and trilaciclib. See List of clinically approved kinase inhibitors|MRC Protein Phosphorylation Ubiquitylation Unit available at www.ppu.mrc.ac.uk/list-clinically-approved-kinase-inhibitors, incorporated herein by reference in its entirety.

In certain embodiments, compounds of the present disclosure, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacizumab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, decitabine, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In certain embodiments, compounds of the present disclosure, or a pharmaceutically acceptable composition thereof, are co-administered with a pharmaceutically acceptable Myt1 kinase inhibitor. In some such embodiments, the Myt1 kinase inhibitor is RP-6306.

In certain embodiments, compounds of the present disclosure, or a pharmaceutically acceptable composition thereof, are co-administered with a pharmaceutically acceptable DNA damaging agent.

In certain embodiments, compounds of the present disclosure, or a pharmaceutically acceptable composition thereof, are co-administered with radiation.

In certain embodiments, compounds of the present disclosure, or a pharmaceutically acceptable composition thereof, are administered in combination with a monoclonal antibody or an siRNA therapeutic.

In certain embodiments, compounds of the present disclosure, or a pharmaceutically acceptable composition thereof, are administered in combination with a targeted therapy selected from (i) an inhibitor of a kinase selected from MET, MEK, mTOR, FLT3, BRAF, KIT, PDGFR, FDFR, PI₃K, EGFR, AKT, and KRAS, (ii) an inhibitor of a fusion kinase like BCR-ABL, ALK, RET and ROS, JAK, CDK₄/6, and KRAS, (iii) epigenetic modulators such as an HDAC inhibitor, (iv) immuno-oncology agents such as those targeting PD1, PDL1, and CTLA4, (v) antibody drug conjugates such as those targeting Her2, CD38, BCMA, CD19, nectin4, trop2, CD79, and CD22, (vi) bispecific T cell engagers (BiTEs), (vii) transcription factor modulators such as those targeting IKZF (i.e., IMiDs, and EZH₂), (viii) steroid receptor modulators such as those targeting AR and ER, and (ix) proteasome inhibitors such as bortezomib, ixazomib, carfilzomib and (x) agents targeting apoptosis such as inhibitors of BCL-2, BCL-XL, MCL1, IAP, or TRAIL/Death Receptor agonists.

In some embodiments, compounds of the present disclosure, or a pharmaceutically acceptable composition thereof, are administered in combination with an inhibitor of a DNA repair protein other than Wee1A kinase or Myt1 kinase. Such inhibitors include those that inhibit one or more of the following CHK1, CHK₂, ATM, ATR, Pol Theta, CDC₇, DNAPK, PLK1, WRN, PARP and Aurora A/B.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another, for example, within one, two, three, four, five, six, seven, eight, nine, ten, eleven, or twelve hours from one another.

In some such embodiments, compounds of the present disclosure, or a pharmaceutically acceptable salt thereof, particularly those compounds that do not have significant Myt1 kinase activity (e.g., compounds not rated A or B in the Myt1 kinase binding assay disclosed in Example 6 herein), are administered as part of a multiple dosage regimen with a pharmaceutically acceptable Myt1 kinase inhibitor. In certain embodiments, compounds of the present disclosure, or a pharmaceutically acceptable composition thereof, are administered as part of a multiple dosage regimen with a Myt1 kinase inhibitor selected from RP-6306.

In some embodiments, compounds of the present disclosure, or a pharmaceutically acceptable salt thereof, particularly those compounds that do not have significant Myt1 kinase activity, are administered to a subject wherein a Myt1 kinase inhibitor is used as the first or second line therapy. In some embodiments, compounds of the present disclosure, or a pharmaceutically acceptable salt thereof, are administered to a subject wherein a Myt1 kinase inhibitor is used as a first line therapy. In some embodiments, compounds of the present disclosure, or a pharmaceutically acceptable salt thereof, are administered to a subject wherein a Myt1 kinase inhibitor selected from RP-6306, is used as a first line therapy.

In some embodiments, compounds of the present disclosure, or a pharmaceutically acceptable salt thereof, particularly those compounds that do not have significant Myt1 kinase activity, are administered to a subject wherein a Myt1 kinase inhibitor is used as a second line therapy. In some embodiments, compounds of the present disclosure, or a pharmaceutically acceptable salt thereof, are administered to a subject wherein a Myt1 kinase inhibitor selected from RP-6306, is used as a second line therapy.

As used herein, the term “combination,” “combined,” “co-administered” and related terms refer to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.001-100 mg/kg body weight/day of an inventive can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions a dosage of between 0.001-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present disclosure provides a method for inhibiting Wee1A kinase in vitro. In some such embodiments, the amount of Wee1A kinase inhibition is assessed based on a competitive ATP-binding assay.

In some embodiments, the present disclosure provides a method for inhibiting Wee1A kinase in a biological sample.

In some embodiments, the present disclosure provides a method for inhibiting both Wee1A kinase and Myt1 kinase in a biological sample.

In some embodiments, the present disclosure provides a method for inhibiting both Wee1A kinase and Myt1 kinase in vitro. In some such embodiments, the amount of Wee1A kinase and Myt1 kinase inhibition are both assessed based on a competitive ATP-binding assay.

In some embodiments, the present disclosure provides a method for assessing Cdk1 phosphorylation in a cell, comprising contacting said cell with a compound described herein. In one embodiment, the contacting step comprises incubating a cell with a compound presented herein. In some such embodiments, the cell is incubated for at least 4 hours. In some embodiments, the cell may comprise a DAOY medulloblastoma cell.

### EXAMPLES

### General Methods

Reagents and solvents were purchased from commercial suppliers and used as received unless otherwise noted. Solvents were dried over molecular sieves 4 Å. Reactions were stirred using magnetic stir bars in glass vials or round bottomed flasks and heated using stirring plates. Solvents were removed on rotary evaporators, vacuum centrifuge, or by freeze-drying. Reaction progress was monitored by LC-MS or thin layer chromatography (TLC). Aluminium-backed TLC plates (60F₂₅₄) were visualized by UV-light (254 nm).

¹H NMR and ¹³C NMR spectra were recorded on a 500 MHz (¹H NMR at 500 MHz. and ¹³C NMR at 126 MHz) Bruker Avance Neo spectrometer equipped with a 5 mm iProbe BBF/H/D probe or on a 400 MHz (¹H NMR at 400 MHz and ¹³C NMR at 101 MHz) Varian Inova spectrometer equipped with a 5 mm 1H/13C auto-switchable gradient-probe at 25 °C. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Spectra were processed using commercial software.

LC-MS were acquired on instruments using reversed phase C18 columns eluting with acetonitrile and water (containing 0.1% TFA or 0.03% ammonia) with mass spectrometers operating in ES (+ or −) ionization mode.

Flash chromatography was performed on silica gel or C18 functionalized silica were performed on an automated flash purification system equipped with a diode array detector (200-400 nm), eluting with gradients of ethyl acetate and petroleum ether or methanol and dichloromethane (containing 0.03% of ammonia.

Purity analyses were performed by HPLC reversed phase C18 columns eluting with acetonitrile and water (containing 0.1% TFA or 0.03% ammonia). UV-traces were recorded at 220 nm. Purifications by preparative HPLC were performed using reversed phase C18 columns eluting with acetonitrile and water (containing 0.1% TFA or 0.03% ammonia).

The stereochemical configurations of enantiomers or diastereomers that were separated using chiral chromatography were assigned arbitrarily. The absolute configurations are unknown.

### ABBREVIATIONS

AA or AcOH acetic acid
ABC ammonium bicarbonate
ACN acetonitrile
aq. aqueous
BOC-anhydride di-tert-butyl dicarbonate
n-BuLi n-butyl lithium
DCM dichloromethane
DIPEA N,N-diisopropylethylamine
DMAP 4-dimethylaminopyridine
DMF N,N-dimethylformamide
DMSO dimethylsulfoxide
Et ethyl
EtOAc ethyl acetate
EtOH ethanol
FA formic acid
GC-MS gas chromatography-mass spectrometry
IPA isopropyl alcohol
IPAm isopropylamine
LDA lithium diisopropylamide
mCPBA meta-chloroperbenzoic acid
MeOH methanol
MTBE methyl tert-butyl ether
NCS N-chlorosuccinimide
NBS N-bromosuccinimide
NMP N-methylpyrrolidine
LC-MS or LCMS liquid chromatography-mass spectroscopy
PdCl₂×dppf 1,1′-bis(diphenylphosphino)ferrocene palladium (II)dichloride
pTSA p-toluenesulfonic acid
RT room temperature, normally 20 to 22 °C.
STAB sodium triacetoxyborohydride
TBAF tetrabutylammonium fluoride
TBS tert-Butyldimethylsilyl
TBSCl tert-Butyldimethylsilyl chloride
TEA triethylamine
TFA trifluoroacetic acid
TFAA trifluoroacetic acid anhydride
THF tetrahydrofuran
TBME tert-butyl methyl ether
TLC thin layer chromatography
tᵣ or Tᵣₑₜ retention time
Triflic anhydride trifluoromethanesulfonic anhydride (Tf₂O)
UPLC Ultra high performance liquid chromatography

### Example 1. Synthesis of 2-allyl-6-((4-chlorophenyl)amino)-1-(6-(piperidin-4-ylamino)pyridin-2-yl)-1,2-dihydro-3H-pyrazolo[3,4-d]pyrimidin-3-one; trifluoroacetic acid (TFA salt of Compound 124) and 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one; trifluoroacetic acid (TFA salt of Compound 138)

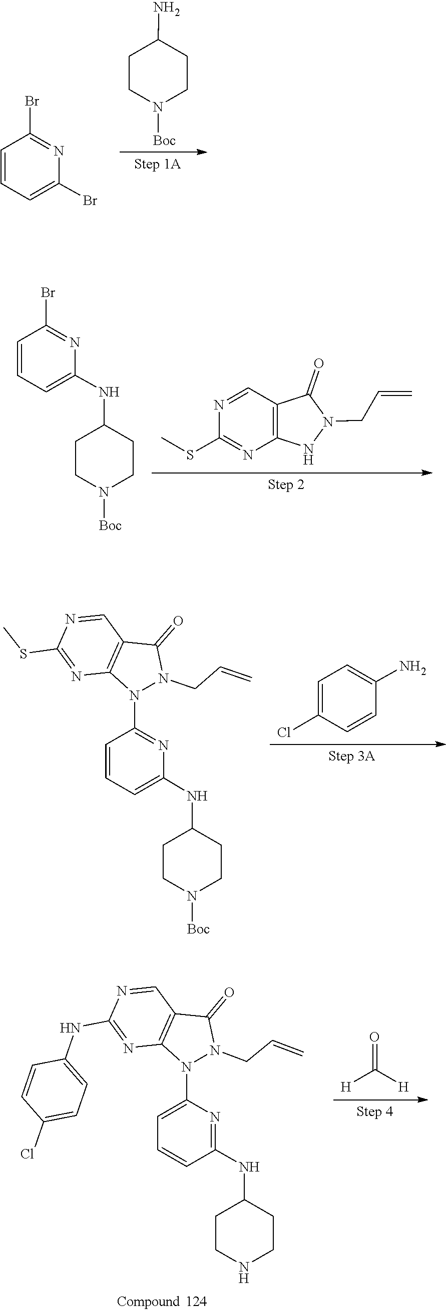

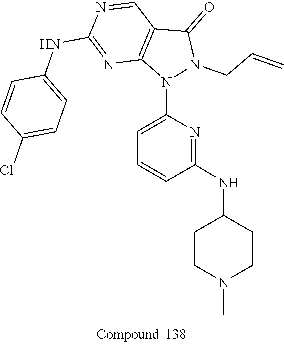

### tert-butyl 4-((6-bromopyridin-2-yl)amino)piperidine-1-carboxylate

N,N-Diisopropylethylamine (3 equivalents) and tert-butyl 4-aminopiperidine-1-carboxylate (1 equivalent) were added to a solution of 2,6-dibromopyridine (1 equivalent) in DMSO (0.8 mol/L) at room temperature. The reaction mixture was heated to 90 °C and stirred for several days until LCMS indicated full conversion. The reaction mixture was diluted with brine and aq. NaHCO₃ (sat.) and extracted with ethyl acetate (×3). The combined organic layers were dried using a phase-separator and concentrated under reduced pressure giving an oil. This material that was either used without further purification or purified by flash chromatography.

Alternatively, cesium carbonate (3 equivalents) and tert-butyl 4-aminopiperidine-1-carboxylate (1 equivalent) were added to a solution of 2,6-dibromopyridine (1 equivalent) in dry dimethyl formamide (0.4 mol/L) and stirred at 100 °C until LCMS indicated full conversion, typically overnight. The reaction mixture was diluted with brine and aq. NaHCO₃ (sat.) and extracted with ethyl acetate (×3). The combined organic layers were dried using a phase-separator and concentrated under reduced pressure giving an oil. This material that was either used without further purification or purified by flash chromatography.

### tert-butyl 4-((6-(2-allyl-6-(methylthio)-3-oxo-2,3-dihydro-1H-pyrazolo[3,4-d]pyrimidin-1-yl)pyridin-2-yl)amino)piperidine-1-carboxylate

CuI (1.2 equivalents) followed by N,N′-dimethylethylenediamine (1 equivalent) was added to a stirred degassed suspension of tert-butyl 4-((6-bromopyridin-2-yl)amino)piperidine-1-carboxylate (1 equivalent), 6-(methylsulfanyl)-2-(prop-2-en-1-yl)-1H,2H,3H-pyrazolo[3,4-d]pyrimidin-3-one (1 equivalent), and cesium carbonate (3 equivalents) in dioxane (0.3 mol/L) at room temperature. The reaction was heated to 90 °C in a closed vial overnight. The reaction was diluted with water and a few drops of aq. ammonia (28%) then extracted with ethyl acetate (×3). The combined organic layers were dried using a phase-separator and concentrated under reduced pressure giving an oil. This material that was either used without further purification or purified by flash chromatography.

### 2-allyl-6-((4-chlorophenyl)amino)-1-(6-(piperidin-4-ylamino)pyridin-2-yl)-1,2-dihydro-3H-pyrazolo[3,4-d]pyrimidin-3-one (Compound 124)

mCPBA (~75%, 1.2 equivalents) was added to a stirred solution of tert-butyl 4-((6-(2-allyl-6-(methylthio)-3-oxo-2,3-dihydro-1H-pyrazolo[3,4-d]pyrimidin-1-yl)pyridin-2-yl)amino)piperidine-1-carboxylate (1 equivalent) in dichloromethane (0.4 mol/L) at room temperature. The mixture was stirred until LCMS indicated full conversion into the corresponding sulfoxide (major) and sulfone (minor), typically within 1 hour.

1-amino-4-chlorobenzene (1 equivalent) was added to the reaction mixture and the resulting mixture was heated to 40 °C until LCMS indicated full conversion, typically overnight. Alternatively, dichloromethane was removed under reduced pressure and the residues redissolved in dry acetonitrile (0.6 mol/L). Then 1-amino-4-chlorobenzene (1 equivalent) was added and the resulting mixture was heated to 60 °C until LCMS indicated full conversion, typically overnight. The reaction mixture was concentrated, the residues were diluted with ethyl acetate, aq. NaOH (1 M) was added, and the product was extracted with ethyl acetate (×3). The combined organic layers were dried using a phase-separator and concentrated under reduced pressure to residues.

Trifluoroacetic acid (10-20% by volume) was added to a stirred solution of the material above (1 equivalent) in dry dichloromethane (0.4 mol/L) at room temperature. The resulting solution was stirred until LCMS indicated full conversion, typically within 1 hour. The reaction mixture was concentrated and purified using reversed phase chromatography. The pure fractions were pooled and lyophilized to give Compound 124. Yield: 29 mg, 49% as yellow solid. HPLC purity (220 nm) 100%.

### 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one (Compound 138)

Formaldehyde (38% in water, 2 equivalents) was added to a stirred a solution of 2-allyl-6-((4-chlorophenyl)amino)-1-(6-(piperidin-4-ylamino)pyridin-2-yl)-1,2-dihydro-3H-pyrazolo[3,4-d]pyrimidin-3-one (1 equivalent) in THF (0.6 mol/L) at room temperature. Diisopropylethylamine (1 equivalent) was added if the starting material was in the form of a trifluoroacetic acid salt. The reaction mixture was stirred for 30 minutes then sodium triacetoxyborohydride (3 equivalents) was added in portions at room temperature. This mixture was stirred until LCMS indicated full conversion in some instances this required addition of more formaldehyde and sodium triacetoxyborohydride. The reaction mixture was concentrated and purified by reversed phase chromatography. The pure fractions were pooled and lyophilized to give Compound 128. Yield: 7.4 mg, 63% as yellow solids. HPLC purity (220 nm) 98%.

### Example 2. Synthesis of 2-allyl-6-((4-chlorophenyl)amino)-1-(6-(piperidin-4-yloxy)pyridin-2-yl)-1,2-dihydro-3H-pyrazolo[3,4-d]pyrimidin-3-one; trifluoroacetic acid (TFA salt of Compound 141) and 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one; trifluoroacetic acid (TFA salt of Compound 142)

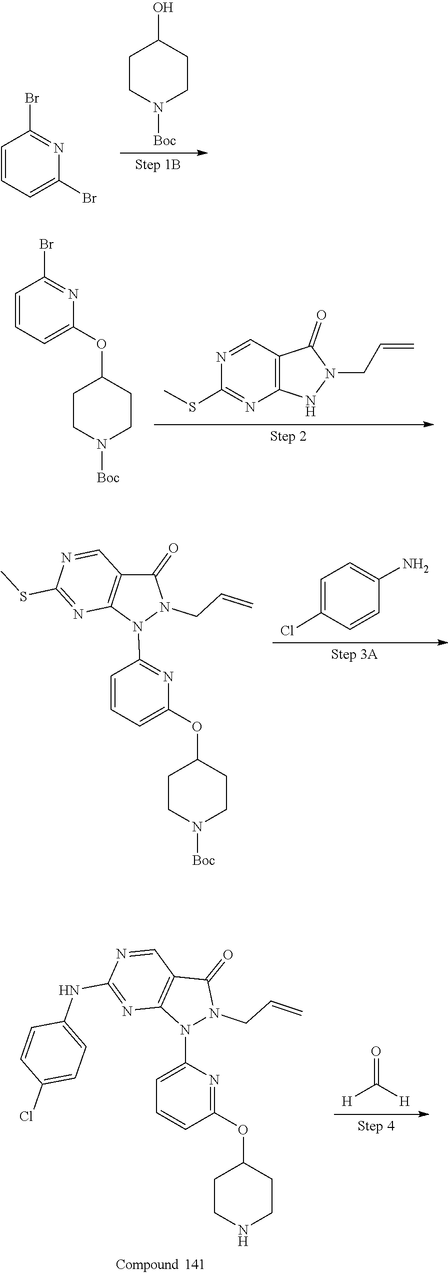

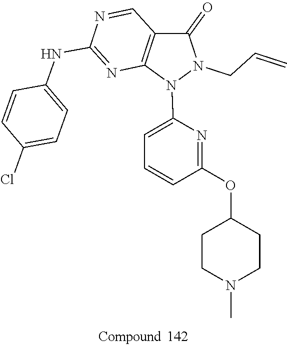

### tert-butyl 4-((6-bromopyridin-2-yl)oxy)piperidine-1-carboxylate

Sodium hydride was added to a stirred solution of tert-butyl 4-hydroxypiperidine-1-carboxylate (1 equivalent) in tetrahydrofuran (0.5 mol/mL) at 0 °C. A solution of 2,6-dibromopyridine (1 equivalent) in THF (0.5 mol/mL) was added to the mixture, upon complete addition the mixture was brought to room temperature and stirred until LCMS indicated full conversion, typically overnight. The reaction mixture was concentrated, diluted with brine and aq. NaHCO₃ (sat.) and extracted with ethyl acetate (×3). The organic layer was washed with brine and then filtered through a phase-separator. The organic layer was concentrated to residues under reduced pressure. The residues were purified by flash chromatography.

### tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate

CuI (1.2 equivalents) followed by N,N′-dimethylethylenediamine (1 equivalent) was added to a stirred degassed suspension of tert-butyl 4-((6-bromopyridin-2-yl)oxy)piperidine-1-carboxylate (1 equivalent), 6-(methylsulfanyl)-2-(prop-2-en-1-yl)-1H,2H,3H-pyrazolo[3,4-d]pyrimidin-3-one (1 equivalent), and cesium carbonate (3 equivalents) in dioxane (0.3 mol/L) at room temperature. The reaction was heated to 90 °C in a closed vial overnight. The reaction was diluted with water and a few drops of aq. ammonia (28%) then extracted with ethyl acetate (×3). The combined organic layers were dried using a phase-separator and concentrated under reduced pressure giving an oil. This material that was either used without further purification or purified by flash chromatography.

### 2-allyl-6-((4-chlorophenyl)amino)-1-(6-(piperidin-4-yloxy)pyridin-2-yl)-1,2-dihydro-3H-pyrazolo[3,4-d]pyrimidin-3-one (Compound 141)

mCPBA (~75%, 1.2 equivalents) was added to a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (1 equivalent) in dichloromethane (0.4 mol/L) at room temperature. The mixture was stirred until LCMS indicated full conversion into the corresponding sulfoxide (major) and sulfone (minor), typically within 1 hour.

1-amino-4-chlorobenzene (1 equivalent) was added to the reaction mixture and the resulting mixture was heated to 40 °C until LCMS indicated full conversion, typically overnight. Alternatively, dichloromethane was removed under reduced pressure and the residues redissolved in dry acetonitrile (0.6 mol/L). Then 1-amino-4-chlorobenzene (1 equivalent) was added and the resulting mixture was heated to 60 °C until LCMS indicated full conversion, typically overnight. The reaction mixture was concentrated, the residues were diluted with ethyl acetate, aq. NaOH (1 M) was added, and the product was extracted with ethyl acetate (×3). The combined organic layers were dried using a phase-separator and concentrated under reduced pressure to residues.

Trifluoroacetic acid (10-20% by volume) was added to a stirred solution of the material above (1 equivalent) in dry dichloromethane (0.4 mol/L) at room temperature. The resulting solution was stirred until LCMS indicated full conversion, typically within 1 hour. The reaction mixture was concentrated and purified using reversed phase chromatography. The pure fractions were pooled and lyophilized to give Compound 141. Yield: 15 mg, 31% as solids. HPLC purity (220 nm) 98%.

### 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one (Compound 142)

Formaldehyde (38% in water, 2 equivalents) was added to a stirred a solution of 2-allyl-6-((4-chlorophenyl)amino)-1-(6-(piperidin-4-yloxy)pyridin-2-yl)-1,2-dihydro-3H-pyrazolo[3,4-d]pyrimidin-3-one (1 equivalent) in THF (0.6 mol/L) at room temperature. Diisopropylethylamine (1 equivalent) was added if the starting material was in the form of a trifluoroacetic acid salt. The reaction mixture was stirred for 30 minutes then sodium triacetoxyborohydride (3 equivalents) was added in portions at room temperature. This mixture was stirred until LCMS indicated full conversion in some instances this required addition of more formaldehyde and sodium triacetoxyborohydride. The reaction mixture was concentrated and purified by reversed phase chromatography. The pure fractions were pooled and lyophilized to give Compound 142. Yield: 42 mg, 63% as solids. HPLC purity (220 nm) 97%.

### Example 3. Synthesis of 1-{6-[(3R)-1-azabicyclo[2.2.2]octan-3-yloxy]pyridin-2-yl}-6-[(1-methyl-1H-pyrazol-4-yl)amino]-2-(prop-2-en-1-yl)-1H,2H,3H-pyrazolo[3,4-d]pyrimidin-3-one; trifluoroacetic acid (TFA salt of Compound 172)

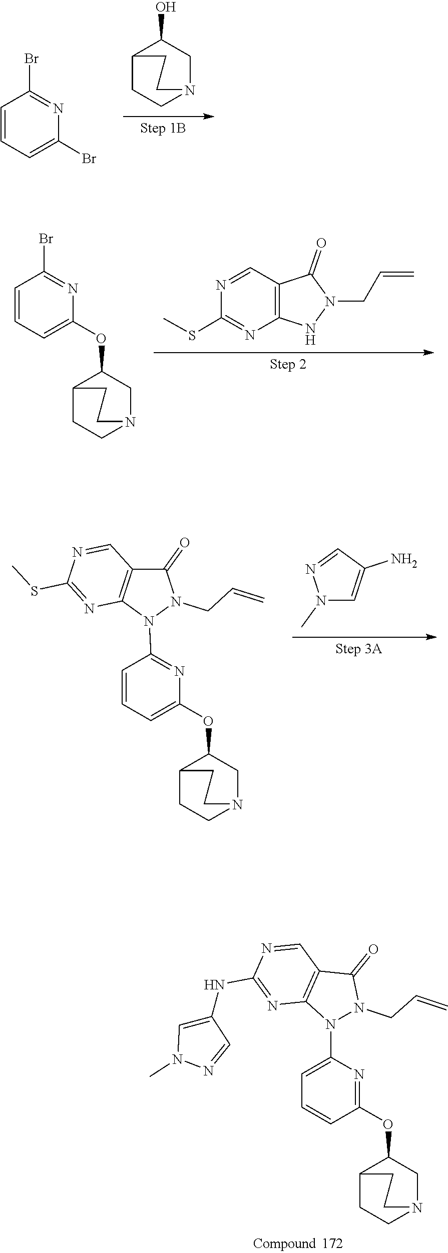

### (R)-3-((6-bromopyridin-2-yl)oxy)quinuclidine

N,N-Diisopropylethylamine (3 equivalents) and (R)-quinuclidin-3-ol (1 equivalent) were added to a solution of 2,6-dibromopyridine (1 equivalent) in DMSO (0.8 mol/L) at room temperature. The reaction mixture was heated to 90 °C and stirred for several days until LCMS indicated full conversion. The reaction mixture was diluted with brine and aq. NaHCO₃ (sat.) and extracted with ethyl acetate (×3). The combined organic layers were dried using a phase-separator and concentrated under reduced pressure giving an oil. This material that was either used without further purification or purified by flash chromatography.

Alternatively, cesium carbonate (3 equivalents) and (R)-quinuclidin-3-ol (1 equivalent) were added to a solution of 2,6-dibromopyridine (1 equivalent) in dry dimethyl formamide (0.4 mol/L) and stirred at 100 °C until LCMS indicated full conversion, typically overnight. The reaction mixture was diluted with brine and aq. NaHCO₃ (sat.) and extracted with ethyl acetate (×3). The combined organic layers were dried using a phase-separator and concentrated under reduced pressure giving an oil. This material that was either used without further purification or purified by flash chromatography.

### (R)-2-allyl-6-(methylthio)-1-(6-(quinuclidin-3-yloxy)pyridin-2-yl)-1,2-dihydro-3H-pyrazolo[3,4-d]pyrimidin-3-one

CuI (1.2 equivalents) followed by N,N′-dimethylethylenediamine (1 equivalent) was added to a stirred degassed suspension of (R)-3-((6-bromopyridin-2-yl)oxy)quinuclidine (1 equivalent), 6-(methylsulfanyl)-2-(prop-2-en-1-yl)-1H,2H,3H-pyrazolo[3,4-d]pyrimidin-3-one (1 equivalent), and cesium carbonate (3 equivalents) in dioxane (0.3 mol/L) at room temperature. The reaction was heated to 90 °C in a closed vial overnight. The reaction was diluted with water and a few drops of aq. ammonia (28%) then extracted with ethyl acetate (×3). The combined organic layers were dried using a phase-separator and concentrated under reduced pressure giving an oil. This material that was either used without further purification or purified by flash chromatography.

### (R)-2-allyl-6-((1-methyl-1H-pyrazol-3-yl)amino)-1-(6-(quinuclidin-3-yloxy)pyridin-2-yl)-1,2-dihydro-3H-pyrazolo[3,4-d]pyrimidin-3-one (Compound 172)

mCPBA (~75%, 1.2 equivalents) was added to a stirred solution of methanesulfonic acid (2 equivalents) and (R)-2-allyl-6-(methylthio)-1-(6-(quinuclidin-3-yloxy)pyridin-2-yl)-1,2-dihydro-3H-pyrazolo[3,4-d]pyrimidin-3-one (1 equivalent) in dichloromethane (0.6 mol/L) at room temperature. The mixture was stirred until LCMS indicated full conversion into the corresponding sulfoxide (major) and sulfone (minor), typically within 1 hour.

1-methyl-1H-pyrazol-4-amine (1 equivalent) was added to the reaction mixture and the resulting mixture was heated to 40 °C until LCMS indicated full conversion, typically overnight. Alternatively, dichloromethane was removed under reduced pressure and the residues redissolved in dry acetonitrile (0.6 mol/L). Then 1-methyl-1H-pyrazol-4-amine (1 equivalent) was added and the resulting mixture was heated to 60 °C until LCMS indicated full conversion, typically overnight. The mixture was concentrated to dryness under reduced pressure and purified by reversed phase chromatography. The pure fractions were pooled and lyophilized to give product. Yield: 53 mg, 38% as yellow solid. HPLC purity (220 nm) 98%.

Other compounds of the disclosure were or can be synthesized by using the synthetic routes described in Examples 1-3, utilizing one or more of the following: a different reactive aromatic ring for the starting 2,6-dibromopyridine, a different amine in Step 1A, a different alcohol in Step 1B, or a different aromatic ring amine in Step 3A or 3B. Those of ordinary skill in the medicinal chemistry art will be able to synthesize the compounds of this disclosure without undue experimentation by adapting the disclosed examples.

### Example 4. Synthesis of 2-allyl-6-(p-bromophenylamino)-1-[6-(1-methyl-4-piperidyloxy)-2-pyridyl]-1,2-dihydro-3H-1,2,5,7-tetraazainden-3-one (Compound 370)

### tert-butyl 4-{6-[6-(4-bromophenylamino)-3-oxo-2-(prop-2-enyl)-1,2-dihydro-3H-1,2,5,7-tetraazainden-1-yl]pyrid-2-yloxy}piperidine-1-carboxylate

mCPBA (<77% pure) (83.1 mg, assumed 0.481 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (200 mg, 0.401 mmol) in DCM (5 mL) at room temperature under nitrogen. The reaction was controlled by LCMS. After 15 min, DCM was removed in vacuo, and the crude solubilized in MeCN (5 ml), then p-bromoaniline (69 mg, 0.401 mmol) was added. The reaction mixture stirred at 60 °C in a closed vial. After 96 h, the reaction mixture was allowed to cool to RT, and mCPBA quenched with 1M NaOH (5 mL) which was added dropwise. The aqueous phase was extracted with EtOAc (3×20 mL) then washed with brine (20 mL). The combined organic layers were dried (MgSO₄) and concentrated under reduced pressure to give the product as a yellow powder. The crude material was dissolved in DCM (5 ml), loaded onto a 10 g silica column and purified by flash chromatography (0-100%, EtOAc:PE) to give tert-butyl 4-{6-[6-(4-bromophenylamino)-3-oxo-2-(prop-2-enyl)-1,2-dihydro-3H-1,2,5,7-tetraazainden-1-yl]pyrid-2-yloxy}piperidine-1-carboxylate [180 mg, 59%] as a pale-yellow solid.

### 6-(4-bromophenylamino)-1-[6-(piperid-4-yloxy)pyrid-2-yl]-2-(prop-2-enyl)-1,2-dihydro-3H-1,2,5,7-tetraazainden-3-one

A solution of tert-butyl 4-{6-[6-(4-bromophenylamino)-3-oxo-2-(prop-2-enyl)-1,2-dihydro-3H-1,2,5,7-tetraazainden-1-yl]pyrid-2-yloxy}piperidine-1-carboxylate (150 mg, 0.236 mmol) in DCM (5 mL) was treated with TFA (2 mL) for 2 h. The solvent was removed in vacuo and the crude was dissolved in EtOAc, washed with sat aq NaHCO₃, brine, dried (MgSO₄), and concentrated. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated giving 6-(4-bromophenylamino)-1-[6-(piperid-4-yloxy)pyrid-2-yl]-2-(prop-2-enyl)-1,2-dihydro-3H-1,2,5,7-tetraazainden-3-one as a white solid. [150 mg].

### 6-(4-bromophenylamino)-1-[6-(1-methylpiperid-4-yloxy)pyrid-2-yl]-2-(prop-2-enyl)-1,2-dihydro-3H-1,2,5,7-tetraazainden-3-one (Compound 370)

6-(4-bromophenylamino)-1-[6-(piperid-4-yloxy)pyrid-2-yl]-2-(prop-2-enyl)-1,2-dihydro-3H-1,2,5,7-tetraazainden-3-one (150 mg, 0.236 mmol) was dissolved in anhydrous THF (5 mL). Formaldehyde (~36% pure, 38.3 μL, 0.471 mmol) and STAB (150 mg, 0.707 mmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with EtOAc (3×20 mL). The combined organic layers were dried (MgSO₄) and concentrated under reduced pressure to give the crude material as a yellow powder. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated giving the title compound [50.9 mg, yield 33.2%].

### Example 5. Synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one (Compound 367)

### tert-butyl 4-{6-[6-(3-bromophenylamino)-2-ethyl-3-oxo-1,2-dihydro-3H-1,2,5,7-tetraazainden-1-yl]-2-pyridyloxy}-1-piperidinecarboxylate

This intermediate was prepared by the same method as tert-butyl 4-{6-[6-(4-bromophenylamino)-3-oxo-2-(prop-2-enyl)-1,2-dihydro-3H-1,2,5,7-tetraazainden-1-yl]pyrid-2-yloxy}piperidine-1-carboxylate.

### 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one (Compound 367)

To a stirred solution of tert-butyl 4-{6-[6-(3-bromophenylamino)-2-ethyl-3-oxo-1,2-dihydro-3H-1,2,5,7-tetraazainden-1-yl]-2-pyridyloxy}-1-piperidinecarboxylate (230 mg, 376 μmol) in a mixture of 1,4-dioxan (7 mL) and water (2 mL), 2M K₂CO₃ (600 μl) and hydroxy (5-pyrimidinyl) boranolate (46.6 mg, 376 μmol) were added. The reaction mass was degassed for 15 minutes. Iron bis[2-(diphenylphosphino)-2,4-cyclopentadien-1-ide]-dichloro-palladamethane (33 mg, 0.12 eq., 45.1 μmol) as was added and the screw cap was tightened on the seal tube. The contents were heated to 100 °C and stirred over night. The reaction mass was cooled to RT, diluted with EtOAc, washed with water followed by brine solution. The organic layer was dried over anhydrous Na₂SO₄ and the solvent was removed under reduced pressure to obtain a crude mass. The collected material (150 mg) was dissolved in DCM:TFA 4:1 v/v. The reaction mixture was stirred at room temperature for 1 h. The mixture was concentrated in vacuo and the residue was dissolved in anhydrous THF (5 mL). Formaldehyde (23.4 μL, 2 eq., 314 μmol) and sodium triacetoxyborohydride (99.8 mg, 3 eq., 471 μmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with ethyl acetate (3×20 mL). The combined organic layers were poured through a phase separator and concentrated under reduced pressure to give the product as a brown-red powder. The resulting material was purified by reverse phase chromatography (Gemini NX—C18, 21*150 mm, water (0.1% TFA)/acetonitrile, gradient over 12 minutes, 25 ml/min). The pure fractions were pooled and concentrated giving the title compound.

### Example 6. Synthesis of 2-ethyl-1-[6-(4-piperidyloxy)-2-pyridyl]-6-[m-(5-pyrimidinyl)phenylamino]-1,2-dihydro-3H-1,2,5,7-tetraazainden-3-one (Compound 366)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 7. Synthesis of 2-allyl-6-[m-(1-imidazolyl)phenylamino]-1-[6-(1-methyl-4-piperidyloxy)-2-pyridyl]-1,2-dihydro-3H-1,2,5,7-tetraazainden-3-one (Compound 365)

### tert-Butyl 4-(6-{2-allyl-6-[m-(1-imidazolyl)phenylamino]-3-oxo-1,2-dihydro-3H-1,2,5,7-tetraazainden-1-yl}-2-pyridyloxy)-1-piperidinecarboxylate

mCPBA (<77% pure) (112 mg, assumed 0.493 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (205 mg, 0.411 mmol) in DCM (5 mL) at room temperature under nitrogen. After 15 min, DCM was evaporate in vacuo, and the crude solubilized in MeCN (5 ml) Then m-(1-imidazolyl) aniline (65.5 mg, 0.411 mmol) and methane sulfonic acid (79 mg, 0.822 mmol) were added. The reaction mixture stirred at 60 °C in a closed vial. After 48 h, the reaction mixture was allowed to cool to RT, and mCPBA quenched with 1M NaOH (5 mL) which was added dropwise. The aqueous phase was extracted with EtOAc (3×20 mL) then washed with brine (20 mL). The combined organic layers were dried (MgSO₄) and concentrated under reduced pressure to give the product as a yellow powder. The crude material was dissolved in DCM (5 ml), loaded onto a 10 g silica column and purified by flash chromatography (0-100%, EtOAc: PE) to give tert-Butyl 4-(6-{2-allyl-6-[m-(1-imidazolyl)phenylamino]-3-oxo-1,2-dihydro-3H-1,2,5,7-tetraazainden-1-yl}-2-pyridyloxy)-1-piperidinecarboxylate [160 mg, 62.7%] as a yellow solid.

### 2-allyl-6-[m-(1-imidazolyl)phenylamino]-1-[6-(4-piperidyloxy)-2-pyridyl]-1,2-dihydro-3H-1,2,5,7-tetraazainden-3-one, trifluoroacetic acid salt

A solution of tert-Butyl 4-(6-{2-allyl-6-[m-(1-imidazolyl)phenylamino]-3-oxo-1,2-dihydro-3H-1,2,5,7-tetraazainden-1-yl}-2-pyridyloxy)-1-piperidinecarboxylate (160 mg, 0.307 mmol) in DCM (5 mL) was treated with TFA (2 mL) for 2 h. The solvent was removed in vacuo and the crude was dissolved in EtOAc, washed with sat aq NaHCO₃, brine, dried (MgSO₄), and concentrated. The resulting material was purified by purified phase chromatography (Gemini NX—C18, 21*150 mm, water (0.1% TFA)/acetonitrile, gradient over 12 minutes, 25 ml/min). The pure fractions were pooled and concentrated giving 2-allyl-6-[m-(1-imidazolyl)phenylamino]-1-[6-(4-piperidyloxy)-2-pyridyl]-1,2-dihydro-3H-1,2,5,7-tetraazainden-3-one as a trifluoroacetic acid salt as a white solid [150 mg].

### 2-allyl-6-[m-(1-imidazolyl)phenylamino]-1-[6-(1-methyl-4-piperidyloxy)-2-pyridyl]-1,2-dihydro-3H-1,2,5,7-tetraazainden-3-one, trifluoroacetic acid salt (Compound 365)

2-allyl-6-[m-(1-imidazolyl)phenylamino]-1-[6-(1-methyl-4-piperidyloxy)-2-pyridyl]-1,2-dihydro-3H-1,2,5,7-tetraazainden-3-one (150 mg, 0.296 mmol) was dissolved in anhydrous THF (5 mL). Formaldehyde (~36% pure, 44 μL, 0.591 mmol) and STAB (188 mg, 0.887 mmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with EtOAc (3×20 mL). The combined organic layers were dried (MgSO₄) and concentrated under reduced pressure to give the crude material as a yellow powder. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated giving 2-allyl-6-[m-(1-imidazolyl)phenylamino]-1-[6-(1-methyl-4-piperidyloxy)-2-pyridyl]-1,2-dihydro-3H-1,2,5,7-tetraazainden-3-one as a trifluoroacetic acid salt [26.9 mg, yield 28%].

### Example 8. Synthesis of 2-allyl-1-[6-(1-methyl-4-piperidyloxy)-2-pyridyl]-6-[m-(1-pyrazolyl)phenylamino]-1,2-dihydro-3H-1,2,5,7-tetraazainden-3-one (Compound 364)

### tert-butyl 4-(6-{3-oxo-2-(prop-2-enyl)-6-[3-(pyrazol-1-yl)phenylamino]-1,2-dihydro-3H-1,2,5,7-tetraazainden-1-yl}pyrid-2-yloxy)piperidine-1-carboxylate

mCPBA (<77% pure) (114 mg, assumed 0.493 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (205 mg, 0.411 mmol) in DCM (5 mL) at room temperature under nitrogen. After 15 min, DCM was evaporated in vacuo, and the crude solubilized in MeCN (5 ml). Then m-(1-pyrazolyl) aniline (65.4 mg, 0.411 mmol) and methane sulfonic acid (79 mg, 0.822 mmol) were added. The reaction mixture stirred at 60 °C in a closed vial. After 48 h, the reaction mixture was allowed to cool to RT, and mCPBA quenched with 1M NaOH (5 mL) which was added dropwise. The aqueous phase was extracted with EtOAc (3×20 mL) then brine 20 mL. The combined organic layers were dried (MgSO₄) and concentrated under reduced pressure to give the product as a yellow powder. The crude material was dissolved in DCM (5 ml), loaded onto a 10 g silica column and purified by flash chromatography (0-100%, EtOAc:PE) to give tert-butyl 4-(6-{3-oxo-2-(prop-2-enyl)-6-[3-(pyrazol-1-yl)phenylamino]-1,2-dihydro-3H-1,2,5,7-tetraazainden-1-yl}pyrid-2-yloxy)piperidine-1-carboxylate [140 mg, 58.5%] as a white solid.

### 2-allyl-1-[6-(4-piperidyloxy)-2-pyridyl]-6-[m-(1-pyrazolyl)phenylamino]-1,2-dihydro-3H-1,2,5,7-tetraazainden-3-one, trifluoroacetic acid salt

A solution of tert-butyl 4-(6-{3-oxo-2-(prop-2-enyl)-6-[3-(pyrazol-1-yl)phenylamino]-1,2-dihydro-3H-1,2,5,7-tetraazainden-1-yl}pyrid-2-yloxy)piperidine-1-carboxylate (140 mg, 0.275 mmol) in DCM (5 mL) was treated with TFA (2 mL) for 2 h. The solvent was removed in vacuo and the crude was dissolved in EtOAc, washed with sat aq NaHCO₃, brine, dried (MgSO₄), and concentrated. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated giving 2-allyl-1-[6-(4-piperidyloxy)-2-pyridyl]-6-[m-(1-pyrazolyl)phenylamino]-1,2-dihydro-3H-1,2,5,7-tetraazainden-3-one as a trifluoroacetic acid salt as a white solid [140 mg].

### 2-allyl-1-[6-(1-methyl-4-piperidyloxy)-2-pyridyl]-6-[m-(1-pyrazolyl)phenylamino]-1,2-dihydro-3H-1,2,5,7-tetraazainden-3-one (Compound 364)

2-allyl-1-[6-(4-piperidyloxy)-2-pyridyl]-6-[m-(1-pyrazolyl)phenylamino]-1,2-dihydro-3H-1,2,5,7-tetraazainden-3-one (140 mg, 0.275 mmol) was dissolved in anhydrous THF (5 mL). Formaldehyde (~36% pure, 40.9 μL, 0.549 mmol) and STAB (175 mg, 0.824 mmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with EtOAc (3×20 mL). The combined organic layers were dried (MgSO₄) and concentrated under reduced pressure to give the crude material as a yellow powder. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated giving 2-allyl-1-[6-(1-methyl-4-piperidyloxy)-2-pyridyl]-6-[m-(1-pyrazolyl)phenylamino]-1,2-dihydro-3H-1,2,5,7-tetraazainden-3-one [25 mg, yield 27%].

### Example 9. Synthesis of 2-allyl-1-[6-(4-piperidyloxy)-2-pyridyl]-6-[m-(1-pyrazolyl)phenylamino]-1,2-dihydro-3H-1,2,5,7-tetraazainden-3-one (Compound 363)

This compound was made using a similar method as described in the synthesis of 2-allyl-1-[6-(1-methyl-4-piperidyloxy)-2-pyridyl]-6-[m-(1-pyrazolyl)phenylamino]-1,2-dihydro-3H-1,2,5,7-tetraazainden-3-one.

### Example 10. Synthesis of 2-allyl-6-(1-isopropyl-1H-indazol-5-ylamino)-1-(6-{1-[(²H₃)methyl]-4-piperidyloxy}-2-pyridyl)-1,2-dihydro-3H-1,2,5,7-tetraazainden-3-one (Compound 354)

Methanol-d4 (31 μl, 0.76 mmol, 4.0 equiv.) and water (2 μl, 0.11 mmol, 0.58 equiv.) were added to a suspension of Dess-Martin periodinane (129 mg, 0.30 mmol, 1.6 equiv.) in DCM (2 ml) at RT and the mixture was stirred for 20 min, after which it was filtered through a 0.45 μm syringe filter. The resulting clear solution of formaldehyde-d2 (1.6 equiv.) was added to a solution of 1-[6-(piperidin-4-yloxy)pyridin-2-yl]-2-(prop-2-en-1-yl)-6-{|1-(propan-2-yl)-1H-indazol-5-yl]amino}-1H,2H,3H-pyrazolo[3,4-d]pyrimidin-3-one (100 mg, 0.19 mmol, 1.0 equiv.) in THF (3 ml) followed by the addition of sodium triacetoxyborodeuteride (81 mg, 0.38 mmol, 2.0 equiv.) and the mixture was stirred at RT. After 1 h, a second portion of formaldehyde-d2 (0.8 equiv.) prepared in the same way as above, followed by sodium triacetoxyborodeuteride (50 mg, 0.23 mmol, 1.2 equiv.) were added. After additional 1 h, LCMS indicated complete conversion of the secondary amine and the mixture was diluted with EtOAc (30 ml) and washed with NaCl (15% aq. 2×30 ml) adjusted to pH 11 with NaOH aq. followed by sat. brine (30 ml), filtered through a phase-separator and concentrated under reduced pressure. The crude material was purified by reversed phase chromatography (Gemini NX—C18, 21*150 mm, water (0.1% TFA)/acetonitrile, gradient over 12 minutes, 25 ml/min) and the pure fractions were lyophilized to give the title compound. Yield: 91 mg TFA salt (73%) as a pale-yellow powder.

### Example 11. Synthesis of 6-(4-biphenylylamino)-2-ethyl-1-[6-(1-methyl-4-piperidyloxy)-2-pyridyl]-1,2-dihydro-3H-1,2,5,7-tetraazainden-3-one (Compound 284)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 12. Synthesis of 2-ethyl-1-[6-(1-methyl-4-piperidyloxy)-2-pyridyl]-6-[m-(3-pyridyl)phenylamino]-1,2-dihydro-3H-1,2,5,7-tetraazainden-3-one (Compound 352)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 13. Synthesis of 2-ethyl-1-[6-(1-methyl-4-piperidyloxy)-2-pyridyl]-6-[p-(1-methyl-4-pyrazolyl)phenylamino]-1,2-dihydro-3H-1,2,5,7-tetraazainden-3-one (Compound 277)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 14. Synthesis of 6-(4-biphenylylamino)-2-ethyl-1-[6-(4-piperidyloxy)-2-pyridyl]-1,2-dihydro-3H-1,2,5,7-tetraazainden-3-one (Compound 272)

This compound was made using a similar method as described in the synthesis of 2-ethyl-1-|6-(1-methyl-4-piperidyloxy)-2-pyridyl|-6-|m-(5-pyrimidinyl)phenylamino|-1,2-dihydro-3H-1,2,5,7-tetraazainden-3-one.

### Example 15. Synthesis of 2-ethyl-6-[p-(1-methyl-4-pyrazolyl)phenylamino]-1-[6-(4-piperidyloxy)-2-pyridyl]-1,2-dihydro-3H-1,2,5,7-tetraazainden-3-one (Compound 271)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 16. Synthesis of 2-allyl-6-(m-bromophenylamino)-1-[6-(1-methyl-4-piperidyloxy)-2-pyridyl]-1,2-dihydro-3H-1,2,5,7-tetraazainden-3-one (Compound 269)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 17. Synthesis of 6-(p-bromophenylamino)-2-ethyl-1-[6-(1-methyl-4-piperidyloxy)-2-pyridyl]-1,2-dihydro-3H-1,2,5,7-tetraazainden-3-one (Compound 268)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 18. Synthesis of 6-(p-bromophenylamino)-2-ethyl-1-[6-(4-piperidyloxy)-2-pyridyl]-1,2-dihydro-3H-1,2,5,7-tetraazainden-3-one (Compound 267)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 19. Synthesis of 2-allyl-6-(1-isopropyl-1H-indazol-5-ylamino)-1-[6-(4-piperidyloxy)-2-pyridyl]-1,2-dihydro-3H-1,2,5,7-tetraazainden-3-one (Compound 265)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one to give the crude free base intermediate (282 mg) as a brown solid of which 9% was purified by prep-HPLC. Yield: 20 mg TFA salt (64%) as a pale-yellow powder.

### Example 20. Synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one (Compound 263)

### tert-butyl 4-(3-(2-allyl-6-amino-3-oxo-2,3-dihydro-1H-pyrazolo[3,4-d]pyrimidin-1-yl)phenoxy)piperidine-1-carboxylate

In a flask, was taken tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (400 mg, 0.755 mmol) and added ammonia in THF (5 ml, 5.00 mmol) at rt under inert atmosphere and stirred for 16 h. Progress of the reaction was monitored by TLC and LCMS. After completion of the reaction, the solvent was evaporated under reduced pressure to get the crude compound which was purified by column chromatography (silica mesh 100-200 at eluent of 80-100% ethyl acetate and hexane) to get the pure compound tert-butyl 4-(3-(2-allyl-6-amino-3-oxo-2,3-dihydro-1H-pyrazolo[3,4-d]pyrimidin-1-yl)phenoxy)piperidine-1-carboxylate (260 mg, 0.346 mmol, 45.7% yield) as a brown solid.

### 2-allyl-6-(2-methoxy-4-pyridylamino)-1-[6-(1-methyl-4-piperidyloxy)-2-pyridyl]-1,2-dihydro-3H-1,2,5,7-tetraazainden-3-one (Compound 263)

CuI (1.2 equivalents) followed by N,N′-dimethylethylenediamine (1 equivalent) was added to a stirred degassed suspension of tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (200 mg), 4-bromo-2-methoxypyridine (1 equivalent), and cesium carbonate (3 equivalents) in dioxane at room temperature. The reaction was heated to 90 °C in a closed vial overnight. The reactions were monitored by LCMS and additional CuI and ligand were added as needed to achieve full conversion. The reaction was diluted with water and a few drops of aq. ammonia (28%) then extracted with ethyl acetate (×3). The combined organic layers were dried using a phase-separator and concentrated under reduced pressure giving an oil. This material was used without further purification.

Trifluoroacetic acid (10-20% by volume) was added to a stirred solution of the material above (1 equivalent) in dry dichloromethane (0.4 mol/L) at room temperature. The resulting solution was stirred until the Boc-amine intermediate was consumed (15 min, LCMS), then partitioned between EtOAc and sat. brine adjusted to pH ca. 12 with aq. NaOH. The organic phase was filtered through a phase-separator and concentrated under reduced pressure to give the crude intermediate as a red solid. Part of this material was purified by reversed phase chromatography (Gemini NX—C18, 21*150 mm, water (0.1% TFA)/acetonitrile, gradient over 12 minutes, 25 ml/min) and the pure fractions were lyophilized to give the amine compound as a TFA salt.

This material was methylated using a method similar as that described for 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 178 mg TFA salt (71%) as a pale-yellow powder.

### Example 21. Synthesis of 2-allyl-6-(1-methyl-1H-indazol-5-ylamino)-1-[2-(4-piperidyloxy)-4-pyrimidinyl]-1,2-dihydro-3H-1,2,5,7-tetraazainden-3-one (Compound 350)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 157 mg TFA salt (62%) as a yellow powder.

### Example 22. Synthesis of 2-allyl-6-(2-methyl-1,3-benzothiazol-6-ylamino)-1-[6-(4-piperidyloxy)-2-pyridyl]-1,2-dihydro-3H-1,2,5,7-tetraazainden-3-one (Compound 264)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 15 mg TFA salt (61%) as a yellow powder.

### Example 23. Synthesis of 2-ethyl-6-[m-(1-methyl-4-pyrazolyl)phenylamino]-1-[6-(4-piperidyloxy)-2-pyridyl]-1,2-dihydro-3H-1,2,5,7-tetraazainden-3-one (Compound 340)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 24. Synthesis of 6-(3-biphenylylamino)-2-ethyl-1-[6-(1-methyl-4-piperidyloxy)-2-pyridyl]-1,2-dihydro-3H-1,2,5,7-tetraazainden-3-one (Compound 339)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 25. Synthesis of 6-(3-biphenylylamino)-2-ethyl-1-[6-(4-piperidyloxy)-2-pyridyl]-1,2-dihydro-3H-1,2,5,7-tetraazainden-3-one (Compound 338)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one.

### Example 26. Synthesis of 2-allyl-6-(2-methyl-1,3-benzothiazol-6-ylamino)-1-[6-(1-methyl-4-piperidyloxy)-2-pyridyl]-1,2-dihydro-3H-1,2,5,7-tetraazainden-3-one (Compound 260)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 65 mg TFA salt (62%) as a pale-yellow powder.

### Example 27. Synthesis of 2-allyl-6-(6-methoxy-3-pyridylamino)-1-[6-(4-piperidyloxy)-2-pyridyl]-1,2-dihydro-3H-1,2,5,7-tetraazainden-3-one (Compound 251)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 28. Synthesis of 2-allyl-6-(6-methoxy-3-pyridylamino)-1-[6-(1-methyl-4-piperidyloxy)-2-pyridyl]-1,2-dihydro-3H-1,2,5,7-tetraazainden-3-one (Compound 250)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 29. Synthesis of 2-allyl-6-(1,3,3a-triaza-5-indenylamino)-1-[6-(1-methyl-4-piperidyloxy)-2-pyridyl]-1,2-dihydro-3H-1,2,5,7-tetraazainden-3-one (Compound 249)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 26 mg TFA salt (56%) as a pale-yellow powder.

### Example 30. Synthesis of 2-allyl-6-(1-isopropyl-1H-indazol-5-ylamino)-1-[6-(1-methyl-4-piperidyloxy)-2-pyridyl]-1,2-dihydro-3H-1,2,5,7-tetraazainden-3-one (Compound 248)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 260 mg TFA salt (81%) as a pale-yellow powder.

### Example 31. Synthesis of 2-allyl-6-(1,3,3a-triaza-5-indenylamino)-1-[6-(4-piperidyloxy)-2-pyridyl]-1,2-dihydro-3H-1,2,5,7-tetraazainden-3-one (Compound 245)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 53 mg TFA salt (22%) as a yellow powder.

### Example 32. Synthesis of 2-allyl-6-(2,1,3-benzothiadiazol-5-ylamino)-1-[6-(1-methyl-4-piperidyloxy)-2-pyridyl]-1,2-dihydro-3H-1,2,5,7-tetraazainden-3-one (Compound 208)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 58 mg TFA salt (64%) as a pale-yellow powder.

### Example 33. Synthesis of 2-allyl-6-(6-isoquinolylamino)-1-[6-(4-piperidyloxy)-2-pyridyl]-1,2-dihydro-3H-1,2,5,7-tetraazainden-3-one (Compound 244)

### tert-butyl 4-{6-[2-allyl-6-(6-isoquinolylamino)-3-oxo-1,2-dihydro-3H-1,2,5,7-tetraazainden-1-yl]-2-pyridyloxy}-1-piperidinecarboxylate

Tert-butyl 4-[6-(2-allyl-6-amino-3-oxo-1,2-dihydro-3H-1,2,5,7-tetraazainden-1-yl)-2-pyridyloxy]-1-piperidinecarboxylate (100 mg, 0.214 mmol), 6-bromoisoquinoline (46.4 mg, 0.214 mmol), Cs₂CO₃ (209 mg, 3 eq.) were mixed and stirred under nitrogen in dioxane (3 mL) for 15 min. A catalytic amount of CuI (48.9 mg, 1.2 eq), and 1,2-bis(methylamino) ethane as a ligand (18.9 mg, 0.214 mmol) were added. The reaction mixture was heated up to 90 °C for 18 h. The progress of the reaction was observed by LCMS. The reaction mixture was cooled to rt, and the crude residue was quenched with saturated aqueous ammonia solution to remove CuI. Then the mixture was extracted with EtOAc (3×10 mL) and brine. The combined organic layers were dried (anhyd. Na₂SO₄) and the solvent was evaporated under reduced pressure. The crude product was dissolved in MeCN (2 ml) and crystalized over 1 h by adding slowly water (2 ml). This gave tert-butyl 4-{6-[2-allyl-6-(6-isoquinolylamino)-3-oxo-1,2-dihydro-3H-1,2,5,7-tetraazainden-1-yl]-2-pyridyloxy}-1-piperidinecarboxylate [33 mg, yield 30%] as an off white solid. LCMS (ESI+), m/z found: 596.

### 2-allyl-6-(6-isoquinolylamino)-1-[6-(4-piperidyloxy)-2-pyridyl]-1,2-dihydro-3H-1,2,5,7-tetraazainden-3-one (Compound 244)

A solution of tert-butyl 4-{6-[2-allyl-6-(6-isoquinolylamino)-3-oxo-1,2-dihydro-3H-1,2,5,7-tetraazainden-1-yl]-2-pyridyloxy}-1-piperidinecarboxylate (33 mg, 0.055 mmol) in DCM (2 mL) was treated with TFA (0.5 mL) for 2 h. The solvent was removed in vacuo and the crude was dissolved in EtOAc, washed with sat aq NaHCO₃, brine, dried (MgSO₄), and concentrated. The resulting material was purified by crystallization in MeCN (2 ml) over 1 h by adding slowly water (2 ml). This gave 2-allyl-6-(6-isoquinolylamino)-1-[6-(4-piperidyloxy)-2-pyridyl]-1,2-dihydro-3H-1,2,5,7-tetraazainden-3-one as a yellow solid [30 mg].

### Example 34. Synthesis of 2-allyl-1-[6-(1-methyl-4-piperidyloxy)-2-pyridyl]-6-(7-quinolylamino)-1,2-dihydro-3H-1,2,5,7-tetraazainden-3-one (Compound 335)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 35. Synthesis of 2-allyl-1-[6-(4-piperidyloxy)-2-pyridyl]-6-(6-quinolylamino)-1,2-dihydro-3H-1,2,5,7-tetraazainden-3-one (Compound 242)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 36. Synthesis of 2-allyl-1-[6-(1-methyl-4-piperidyloxy)-2-pyridyl]-6-(6-quinoxalinylamino)-1,2-dihydro-3H-1,2,5,7-tetraazainden-3-one (Compound 241)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 37. Synthesis of 2-allyl-6-(7-isoquinolylamino)-1-[6-(4-piperidyloxy)-2-pyridyl]-1,2-dihydro-3H-1,2,5,7-tetraazainden-3-one (Compound 240)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 38. Synthesis of 2-allyl-1-[6-(4-piperidyloxy)-2-pyridyl]-6-(6-quinoxalinylamino)-1,2-dihydro-3H-1,2,5,7-tetraazainden-3-one (Compound 239)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 39. Synthesis of 1-{6-[(S)-1-methyl-3-piperidyloxy]-2-pyridyl}-2-allyl-6-(1-methyl-1H-indazol-5-ylamino)-1,2-dihydro-3H-1,2,5,7-tetraazainden-3-one (Compound 332)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 61 mg TFA salt (82%) as a pale-yellow powder.

### Example 40. Synthesis of 2-allyl-6-(5-fluoro-3-pyridylamino)-1-[6-(4-piperidyloxy)-2-pyridyl]-1,2-dihydro-3H-1,2,5,7-tetraazainden-3-one (Compound 329)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 157 mg TFA salt (62%) as a yellow powder. Yield: 10 mg TFA salt (46%).

### Example 41. Synthesis of 6-(1,3a-diaza-5-indenylamino)-2-allyl-1-[6-(1-methyl-4-piperidyloxy)-2-pyridyl]-1,2-dihydro-3H-1,2,5,7-tetraazainden-3-one (Compound 328)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 70 mg TFA salt (80%) as a yellow solid.

### Example 42. Synthesis of 2-allyl-6-(5-fluoro-3-pyridylamino)-1-[6-(1-methyl-4-piperidyloxy)-2-pyridyl]-1,2-dihydro-3H-1,2,5,7-tetraazainden-3-one (Compound 327)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 157 mg TFA salt (62%) as a yellow powder. Yield: 125 mg TFA salt (83%) as a yellow solid.

### Example 43. Synthesis of 2-allyl-6-(7-isoquinolylamino)-1-[6-(1-methyl-4-piperidyloxy)-2-pyridyl]-1,2-dihydro-3H-1,2,5,7-tetraazainden-3-one (Compound 326)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 44. Synthesis of 6-(1,3a-diaza-5-indenylamino)-2-allyl-1-[6-(4-piperidyloxy)-2-pyridyl]-1,2-dihydro-3H-1,2,5,7-tetraazainden-3-one (Compound 324)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 100 mg TFA salt (42%).

### Example 45. Synthesis of 2-allyl-6-(5-chloro-3-pyridylamino)-1-[6-(1-methyl-4-piperidyloxy)-2-pyridyl]-1,2-dihydro-3H-1,2,5,7-tetraazainden-3-one (Compound 323)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one.

### Example 46. Synthesis of 2-allyl-6-(1-methyl-1H-indazol-5-ylamino)-1-[6-(1-propyl-4-piperidyloxy)-2-pyridyl]-1,2-dihydro-3H-1,2,5,7-tetraazainden-3-one (Compound 322)

2-allyl-6-(1-methyl-1H-indazol-5-ylamino)-1-[6-(4-piperidyloxy)-2-pyridyl]-1,2-dihydro-3H-1,2,5,7-tetraazainden-3-one was made with a similar method as described in the synthesis of 2-allyl-6-((4-chlorophenyl)amino)-1-(6-(piperidin-4-yloxy)pyridin-2-yl)-1,2-dihydro-3H-pyrazolo[3,4-d]pyrimidin-3-one.

Propanol (37.7 μL, 0.502 mmol) was added to a suspension of Dess-Martin periodinane (63.9 mg, 0.151 mmol) in DCM (4 mL) followed by 9 μl H₂O. The mixture was stirred for 30 mins until it became a white suspension which was filtrated. 2 ml of the filtered solution was added to a stirred solution at room temperature of 2-allyl-6-(1-methyl-1H-indazol-5-ylamino)-1-[6-(4-piperidyloxy)-2-pyridyl]-1,2-dihydro-3H-1,2,5,7-tetraazainden-3-one (50 mg, 0.1 mmol) in 2 ml THF. STAB (63.9 mg, 0.301 mmol) was added. The mixture was stirred at RT overnight. The mixture was diluted with EtOAc (20 ml) and washed with a mixture of brine made slightly basic with NaOH (2×20 ml). The organic phase was dried through a phase separator and concentrated in vacuo. The crude material was purified with reversed phase chromatography (Gemini NX—C18, 21*150 mm, water (0.1% TFA)/acetonitrile, gradient over 12 minutes, 25 ml/min). The pure fractions were pooled and concentrated to give the 2-allyl-6-(1-methyl-1H-indazol-5-ylamino)-1-[6-(1-propyl-4-piperidyloxy)-2-pyridyl]-1,2-dihydro-3H-1,2,5,7-tetraazainden-3-one as a trifluoroacetic acid salt (26 mg).

### Example 47. Synthesis of 2-allyl-1-[6-(1-ethyl-4-piperidyloxy)-2-pyridyl]-6-(1-methyl-1H-indazol-5-ylamino)-1,2-dihydro-3H-1,2,5,7-tetraazainden-3-one (Compound 321)

EtOH (29.3 μL, 0.502 mmol) was added to a suspension of Dess-Martin periodinane (63.9 mg, 0.151 mmol) in DCM (4 mL) followed by 9 μl H₂O. The mixture stirred for 30 mins until it becomes a suspension which was filtrated. 2 ml of the filtered solution was added to a stirred solution at room temperature of 2-allyl-6-(1-methyl-1H-indazol-5-ylamino)-1-[6-(4-piperidyloxy)-2-pyridyl]-1,2-dihydro-3H-1,2,5,7-tetraazainden-3-one (50 mg, 0.1 mmol) in 2 ml THF. STAB (42.6 mg, 0.201 mmol) was added. The mixture was stirred at RT overnight. The mixture was diluted with EtOAc (20 ml) and washed with a mixture of brine made slightly basic with NaOH (2×20 ml). The organic phase was dried through a phase separator and concentrated in vacuo. The crude material was purified with reversed phase chromatography (Gemini NX—C18, 21*150 mm, water (0.1% TFA)/acetonitrile, gradient over 12 minutes, 25 ml/min). The pure fractions were pooled and concentrated to give the 2-allyl-6-(1-methyl-1H-indazol-5-ylamino)-1-[6-(1-propyl-4-piperidyloxy)-2-pyridyl]-1,2-dihydro-3H-1,2,5,7-tetraazainden-3-one as a trifluoroacetic acid salt (25 mg).

### Example 48. Synthesis of 2-allyl-6-(1,2-benzisothiazol-5-ylamino)-1-[6-(1-methyl-4-piperidyloxy)-2-pyridyl]-1,2-dihydro-3H-1,2,5,7-tetraazainden-3-one (Compound 320)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 58 mg TFA salt (64%) as a pale-yellow powder.

### Example 49. Synthesis of 2-allyl-1-[m-(1-methyl-4-piperidyloxy)phenyl]-6-(2-methyl-4-pyridylamino)-1,2-dihydro-3H-1,2,5,7-tetraazainden-3-one (Compound 219)

To a stirred solution of 2-allyl-6-((2-methylpyridin-4-yl)amino)-1-(3-(piperidin-4-yloxy)phenyl)-1,2-dihydro-3H-pyrazolo[3,4-d]pyrimidin-3-one (350 mg, 0.765 mmol) in THF (5 ml) was added formaldehyde (310 mg, 3.82 mmol) at 25 °C, then reaction mixture stirred at 25 °C for 5 min. Then STAB (486 mg, 2.295 mmol) was added portion-wise. After the complete addition of STAB, the reaction mixture was stirred at 25 °C for 20 minutes. Progress of the reaction was monitored by TLC and LCMS. Then, the reaction mixture was quenched with TFA, followed by NH₃ in MeOH diluted with water and extracted with 10% MeOH in DCM (2×100 mL). The combined organic extracts were washed with NaHCO₃, dried over anhydrous Na₂SO₄, filtered and concentrated under reduced pressure to afford crude compound which was purified by Prep HPLC to get the pure compound 2-allyl-1-(3-((1-methylpiperidin-4-yl)oxy)phenyl)-6-((2-methylpyridin-4-yl)amino)-1,2-dihydro-3H-pyrazolo[3,4-d]pyrimidin-3-one (66 mg, 0.136 mmol, 17.75% yield) as an off-white solid.

### Example 50. Synthesis of 2-allyl-6-(p-fluorophenylamino)-1-[6-(1-methyl-4-azepanyloxy)-2-pyridyl]-1,2-dihydro-3H-1,2,5,7-tetraazainden-3-one (Compound 317)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 51. Synthesis of 2-allyl-6-(2-methyl-1,3-benzoxazol-5-ylamino)-1-[6-(4-piperidyloxy)-2-pyridyl]-1,2-dihydro-3H-1,2,5,7-tetraazainden-3-one (Compound 232)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 13.5 mg TFA salt (50%) as a yellow powder.

### Example 52. Synthesis of 2-allyl-6-(2-methyl-1,3-benzoxazol-6-ylamino)-1-[6-(4-piperidyloxy)-2-pyridyl]-1,2-dihydro-3H-1,2,5,7-tetraazainden-3-one (Compound 316)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 17.6 mg TFA salt (65%) as a yellow powder.

### Example 53. Synthesis of 2-allyl-6-(2-methyl-1,3-benzoxazol-5-ylamino)-1-[6-(1-methyl-4-piperidyloxy)-2-pyridyl]-1,2-dihydro-3H-1,2,5,7-tetraazainden-3-one (Compound 231)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 19 mg TFA salt (80%) as a pale-yellow powder.

### Example 54. Synthesis of 2-allyl-6-(2-methyl-4-pyridylamino)-1-[m-(4-piperidyloxy)phenyl]-1,2-dihydro-3H-1,2,5,7-tetraazainden-3-one (Compound 222)

### tert-butyl 4-(3-bromophenoxy)piperidine-1-carboxylate

To a stirred solution of tert-butyl 4-hydroxypiperidine-1-carboxylate (7.48 g, 37.1 mmol) in DMF (50 ml) was added NaH (2.285 g, 57.1 mmol) at 0 °C under inert atmosphere and stirred for 1 hour at 50 °C. Later, the flask was cooled to room temperature, and 1-bromo-3-fluorobenzene (5 g, 28.6 mmol) was dissolved in DMF (10 ml) and added to the reaction mass and stirred for 3 h at 70 °C. The progress of the reaction was monitored by LCMS and TLC. After completion of the reaction, the reaction mass was quenched with ice-cold water and extracted with EtOAc (500 mL), The organic layer was washed with brine, dried over anhydrous Na₂SO₄, filtered, and concentrated under reduced pressure to get the crude compound which was purified by column chromatography (silica mesh 100-200 at eluent of 10-20% ethyl acetate and hexane) to give tert-butyl 4-(3-bromophenoxy)piperidine-1-carboxylate (6.8 g, 14.89 mmol, 52.1% yield) as yellow gummy liquid. LCMS m/z found: 300.0 (M−56).

### N-(6-(2-allyl-6-(methylthio)-3-oxo-2,3-dihydro-1H-pyrazolo[3,4-d]pyrimidin-1-yl)pyridin-2-yl)-2-cyano-N-methylacetamide

A stirred solution of 2-allyl-6-(methylthio)-1,2-dihydro-3H-pyrazolo[3,4-d]pyrimidin-3-one (420 mg, 1.890 mmol), tert-butyl 4-(3-bromophenoxy)piperidine-1-carboxylate (808 mg, 2.268 mmol), K₂CO₃ (783 mg, 5.67 mmol) and N,N′-dimethylethylenediamine (0.203 ml, 1.890 mmol) in dioxane (5 ml) was degassed for 20 minutes at room temperature under inert atmosphere. CuI (359 mg, 1.890 mmol) was added and the mixture again degassed for 5 minutes then stirred for 16 h at 110 °C. The progress of the reaction was monitored by TLC and LCMS. After completion of the reaction, the reaction mixture was filtered through celite and washed with 10% MeOH in DCM (150 mL). The collected fractions were concentrated under reduced pressure to get a crude compound which was purified by flash column chromatography (SiO₂/230-400 mesh; 20-50% ethyl acetate-pet ether) to give tert-butyl 4-(3-(2-allyl-6-(methylthio)-3-oxo-2,3-dihydro-1H-pyrazolo[3,4-d]pyrimidin-1-yl)phenoxy)piperidine-1-carboxylate (65 mg, 0.108 mmol, 5.74% yield) as an off-white solid.

### tert-butyl-4-(3-(2-allyl-6-(methylsulfonyl)-3-oxo-2,3-dihydro-1H-pyrazolo[3,4-d]pyrimidin-1-yl)phenoxy)piperidine-1-carboxylate

To a stirred solution of tert-butyl 4-(3-(2-allyl-6-(methylthio)-3-oxo-2,3-dihydro-1H-pyrazolo[3,4-d]pyrimidin-1-yl)phenoxy)piperidine-1-carboxylate (60 mg, 0.121 mmol) in DCM (2 ml) was added m-CPBA (41.6 mg, 0.241 mmol). The mixture was stirred for 2 h, at room temperature. The progress of the reaction was monitored by TLC and LCMS. After completion of the reaction, the reaction mixture was quenched with sodium bicarbonate solution, then extracted with 10% MeOH in DCM. The organic layer was dried over sodium sulfate, and concentrated under reduced pressure to give the crude tert-butyl-4-(3-(2-allyl-6-(methylsulfonyl)-3-oxo-2,3-dihydro-1H-pyrazolo[3,4-d]pyrimidin-1-yl)phenoxy)piperidine-1-carboxylate (60 mg, 0.113 mmol, 94% yield). This crude compound, as such, is taken for the next step without any further purification.

### tert-butyl-4-(3-(2-allyl-6-amino-3-oxo-2,3-dihydro-1H-pyrazolo[3,4-d]pyrimidin-1-yl)phenoxy)piperidine-1-carboxylate

In a flask, tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (400 mg, 0.755 mmol) was charged and ammonia in THF (5 ml, 5.00 mmol) at rt under inert atmosphere and stirred for 16 h. Progress of the reaction was monitored by TLC and LCMS. After completion of the reaction, solvent was evaporated under reduced pressure to get the crude compound which was purified by column chromatography (silica mesh 100-200 at eluent of 80-100% ethyl acetate and hexane) to give tert-butyl 4-(3-(2-allyl-6-amino-3-oxo-2,3-dihydro-1H-pyrazolo[3,4-d]pyrimidin-1-yl)phenoxy)piperidine-1-carboxylate (260 mg, 0.346 mmol, 45.7% yield) as a brown solid.

### tert-butyl-4-(3-(2-allyl-6-((2-methylpyridin-4-yl)amino)-3-oxo-2,3-dihydro-1H-pyrazolo[3,4-d]pyrimidin-1-yl)phenoxy)piperidine-1-carboxylate

A stirred solution of tert-butyl 4-(3-(2-allyl-6-amino-3-oxo-2,3-dihydro-1H-pyrazolo[3,4-d]pyrimidin-1-yl)phenoxy)piperidine-1-carboxylate (300 mg, 0.643 mmol), 4-bromo-2-methylpyridine (133 mg, 0.772 mmol), K₂CO₃ (267 mg, 1.929 mmol) and N,N′-dimethylethylenediamine (56.6 mg, 0.643 mmol) in dioxane (4 ml) was degassed with N₂ for 30 minutes. To this mixture was added Copper(I) iodide (122 mg, 0.643 mmol) and the mixture again degassed for 5 minutes. The mixture was stirred at 110 °C for 16 h. Progress of the reaction was monitored by TLC and LCMS. After completion of the reaction, the reaction mass was filtered through celite, and the crude compound was washed with ethyl acetate (100 mL). The combined organic layers were dried over anhydrous Na₂SO₄, concentrated under vacuum to get the crude compound which was purified by column chromatography (silica mesh 100-200 at eluent of 80-100% ethyl acetate and hexane) to give tert-butyl 4-(3-(2-allyl-6-((2-methylpyridin-4-yl)amino)-3-oxo-2,3-dihydro-1H-pyrazolo[3,4-d]pyrimidin-1-yl)phenoxy)piperidine-1-carboxylate (140 mg, 0.208 mmol, 32.4% yield) as an off-white solid. LCMS m/z found: 558.5 (M+H).

### 2-allyl-6-((2-methylpyridin-4-yl)amino)-1-(3-(piperidin-4-yloxy)phenyl)-1,2-dihydro-3H-pyrazolo[3,4-d]pyrimidin-3-one (Compound 222)

To a stirred solution of tert-butyl 4-(3-(2-allyl-6-((2-methylpyridin-4-yl)amino)-3-oxo-2,3-dihydro-1H-pyrazolo[3,4-d]pyrimidin-1-yl)phenoxy)piperidine-1-carboxylate (40 mg, 0.071 mmol) in dioxane (1 ml) was added HCl in dioxane (0.2 ml, 0.800 mmol) at 0 °C under inert atmosphere. The mixture was then stirred for 16 h at rt. Progress of the reaction was monitored by TLC and LCMS. After completion of the reaction, the solvent was evaporated under reduced pressure to get the crude compound which was washed with n-hexane, and the obtained crude compound was then purified by prep HPLC (Column: X-select CSH C18, Mobile phase A: Water (with 0.1% AA), Mobile phase B: Acetonitrile, Flow rate-15.0 mL/Min, Rt-12.8) to give 2-allyl-6-((2-methylpyridin-4-yl)amino)-1-(3-(piperidin-4-yloxy)phenyl)-1,2-dihydro-3H-pyrazolo[3,4-d]pyrimidin-3-one (14 mg, 0.030 mmol, 42.65% yield).

### Example 55. Synthesis of 2-allyl-1-[6-(4-piperidyloxy)-2-pyridyl]-6-(3-pyridylamino)-1,2-dihydro-3H-1,2,5,7-tetraazainden-3-one (Compound 230)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 93 mg as a white powder.

### Example 56. Synthesis of 2-allyl-1-[6-(1-methyl-4-piperidyloxy)-2-pyridyl]-6-(3-pyridylamino)-1,2-dihydro-3H-1,2,5,7-tetraazainden-3-one (Compound 229)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 96 mg TFA salt (87%) as a pale-yellow powder.

### Example 57. Synthesis of 2-allyl-1-[6-(4-azepanyloxy)-2-pyridyl]-6-(p-fluorophenylamino)-1,2-dihydro-3H-1,2,5,7-tetraazainden-3-one (Compound 315)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 58. Synthesis of 2-allyl-6-(1,2-benzisothiazol-5-ylamino)-1-[6-(4-piperidyloxy)-2-pyridyl]-1,2-dihydro-3H-1,2,5,7-tetraazainden-3-one (Compound 228)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 59. Synthesis of 2-allyl-1-[2-(1-methyl-4-piperidylamino)-4-pyrimidinyl]-6-[p-(2,2,2-trifluoroethoxy)phenylamino]-1,2-dihydro-3H-1,2,5,7-tetraazainden-3-one (Compound 314)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 49 mg TFA salt (57%) as a white powder.

### Example 60. Synthesis of 2-allyl-6-(1-methyl-1H-indazol-5-ylamino)-1-[2-(1-methyl-4-piperidylamino)-4-pyrimidinyl]-1,2-dihydro-3H-1,2,5,7-tetraazainden-3-one (Compound 313)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 24.5 mg TFA salt (40%) as a yellow powder.

### Example 61. Synthesis of 2-allyl-6-(p-fluorophenylamino)-1-[2-(1-methyl-4-piperidylamino)-4-pyrimidinyl]-1,2-dihydro-3H-1,2,5,7-tetraazainden-3-one (Compound 312)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 40 mg TFA salt (54%) as a white powder.

### Example 62. Synthesis of 2-allyl-1-[m-(1-methyl-4-piperidyloxy)phenyl]-6-(1-methyl-4-pyrazolylamino)-1,2-dihydro-3H-1,2,5,7-tetraazainden-3-one (Compound 221)

### tert-butyl 4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate

To a stirred solution of tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (80 mg, 0.151 mmol) in acetic acid (3 mL) was added 1-methyl-1H-pyrazol-4-amine (14.67 mg, 0.151 mmol) at 25 °C, then reaction mixture was allowed to stir at 25 °C for 16 h. The progress of the reaction was monitored by TLC and LCMS. After completion of the reaction. The reaction mixture was concentrated under reduced pressure and then diluted with 10% aq. sodium bicarbonate and then extracted with 10% MeOH in DCM. The combined organic layers were dried over anhydrous sodium sulphate, filtered and concentrated under reduced pressure to give crude compound which was purified using column chromatography (silica gel, mesh 100-200 at eluent of 5-10% MeOH in DCM) to give tert-butyl 4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate (60 mg, 0.082 mmol, 54.5% yield) as an off-white solid. LCMS m/z found: 547.6 (M+1).

### 2-allyl-6-((1-methyl-1H-pyrazol-4-yl)amino)-1-(3-(piperidin-4-yloxy)phenyl)-1,2-dihydro-3H-pyrazolo[3,4-d]pyrimidin-3-one

To a stirred solution of tert-butyl 4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate (60 mg, 0.110 mmol) in 1,4-dioxane (5 ml) was added 4M HCl (0.329 mL, 1.317 mmol) in dioxane at 0 °C under inert atmosphere. Then the reaction mass was allowed to stir at room temperature for 8 h. The progress of the reaction was monitored by TLC and LCMS. After completion of the reaction, the reaction mass was concentrated under reduced pressure to give the crude compound which was purified by Prep-HPLC (Column: X-select CSH C18, Mobile phase A: Water (with 0.1% FA), Mobile phase B: Acetonitrile (with 0.1% FA), Flow rate-15.0 mL/Min, Rt-12.8) to give 2-allyl-6-((1-methyl-1H-pyrazol-4-yl)amino)-1-(3-(piperidin-4-yloxy)phenyl)-1,2-dihydro-3H-pyrazolo[3,4-d]pyrimidin-3-one (8 mg, 0.018 mmol, 16% yield) as a brown solid.

### 2-allyl-1-[m-(1-methyl-4-piperidyloxy)phenyl]-6-(1-methyl-4-pyrazolylamino)-1,2-dihydro-3H-1,2,5,7-tetraazainden-3-one (Compound 221)

To a stirred solution of 2-allyl-6-((1-methyl-1H-pyrazol-4-yl)amino)-1-(3-(piperidin-4-yloxy)phenyl)-1,2-dihydro-3H-pyrazolo[3,4-d]pyrimidin-3-one (100 mg, 0.224 mmol) in THF (5 mL) was added 37%, aq formaldehyde (0.083 mL, 1.120 mmol) at 25 °C, and the mixture was stirred for 10 minutes. Then STAB (142 mg, 0.672 mmol) was added portion wise. After complete addition of STAB, the reaction mixture was stirred at 25 °C for 3 h. The progress of reaction was monitored by UPLC. After completion of the reaction, the reaction mixture was quenched with TFA followed by ammonia in MeOH. The reaction mixture was diluted with water and extracted with 10% MeOH in DCM. The combined organic extracts were washed with aq. sodium bicarbonate, dried over anhydrous sodium sulphate, filtered, and concentrated under reduced pressure to afford crude compound which was purified by Prep-HPLC (Column: X-select CSH C18, Mobile Phase A: Ammonium acetate in water, Mobile Phase B: Acetonitrile, Flowrate: 15.0 mL/Min, Rt-10.8) to give 2-allyl-6-((1-methyl-1H-pyrazol-4-yl)amino)-1-(3-((1-methylpiperidin-4-yl)oxy)phenyl)-1,2-dihydro-3H-pyrazolo[3,4-d]pyrimidin-3-one.

### Example 63. Synthesis of 2-allyl-6-(2-methyl-1,3-benzoxazol-6-ylamino)-1-[6-(1-methyl-4-piperidyloxy)-2-pyridyl]-1,2-dihydro-3H-1,2,5,7-tetraazainden-3-one (Compound 311)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 184 mg TFA salt (84%) as a pale-yellow powder.

### Example 64. Synthesis of 1-{m-[N-methyl (1-methyl-4-piperidyl)amino]phenyl}-2-allyl-6-(1-methyl-1H-indazol-5-ylamino)-1,2-dihydro-3H-1,2,5,7-tetraazainden-3-one (Compound 225)

### 2-allyl-6-(methylthio)-1-(3-nitrophenyl)-1,2-dihydro-3H-pyrazolo[3,4-d]pyrimidin-3-one

To a stirred solution of 2-allyl-6-(methylthio)-1,2-dihydro-3H-pyrazolo[3,4-d]pyrimidin-3-one (1.2 g, 5.40 mmol) in dichloromethane (15 mL) was added 3-nitrophenyl boronic acid (1.352 g, 8.10 mmol), sodium carbonate (1.707 g, 16.20 mmol) and copper (II) acetate (0.49 g, 2.70 mmol) followed by pyridine (0.169 g, 1.080 mmol) at room temperature. The mixture was heated to 70 °C and stirred for 16 h. The progress of the reaction was monitored by TLC and LCMS. After completion of the reaction, the mixture was filtered through celite and washed with 10% MeOH in DCM (2×100 mL). The combined organic layers were dried over anhydrous Na₂SO₄, concentrated under reduced pressure to give the crude compound, which was purified by column chromatography (silica mesh 100-200 at eluent of 70-100% Ethyl acetate and hexane) to give 2-allyl-6-(methylthio)-1-(3-nitrophenyl)-1,2-dihydro-3H-pyrazolo[3,4-d]pyrimidin-3-one (700 mg, 1.794 mmol, 33.2% yield) as a white solid. LCMS m/z found: 344.2 (M+H).

### 2-allyl-6-(methylsulfonyl)-1-(3-nitrophenyl)-1,2-dihydro-3H-pyrazolo[3,4-d]pyrimidin-3-one

To a stirred solution of 2-allyl-6-(methylthio)-1-(3-nitrophenyl)-1,2-dihydro-3H-pyrazolo[3,4-d]pyrimidin-3-one (50 mg, 0.146 mmol) in DCM (10 ml) was added mCPBA (53.7 mg, 0.218 mmol) at room temperature under inert atmosphere. The mixture was stirred for 2 h. The progress of the reaction was monitored by TLC and UPLC. After completion of the reaction, the reaction mixture was quenched with aq. 10% sodium bicarbonate solution (10 mL) and extracted with 10% MeOH in DCM (2×50 mL). The combined organic layers were washed with brine (100 mL), dried over anhydrous sodium sulphate, filtered and concentrated under reduced pressure to give the crude compound 2-allyl-6-(methylsulfonyl)-1-(3-nitrophenyl)-1,2-dihydro-3H-pyrazolo[3,4-d]pyrimidin-3-one (30 mg, 0.024 mmol, 16.47% yield) as an off-white solid. This crude compound was taken as such for the next step without further purification.

### 2-allyl-6-((1-methyl-1H-indazol-5-yl)amino)-1-(3-nitrophenyl)-1,2-dihydro-3H-pyrazolo[3,4-d]pyrimidin-3-one

To a stirred solution of 2-allyl-6-(methylsulfonyl)-1-(3-nitrophenyl)-1,2-dihydro-3H-pyrazolo[3,4-d]pyrimidin-3-one (0.7 g, 2.039 mmol) in AcOH (5 mL) was added 1-methyl-1H-indazol-5-amine (0.3 g, 2.039 mmol) and allowed to stirred for 16 h at room temperature. The progress of the reaction was monitored by TLC and UPLC. After completion of the reaction, the solvent was evaporated under reduced pressure and the residue obtained was quenched with 10% aq sodium bicarbonate solution (100 mL). The resulting mixture was extracted with ethyl acetate (2×300 mL). The combined organic extract was washed with brine (100 mL), dried over anhydrous sodium sulphate, filtered and concentrated under reduced pressure to give the crude compound which was purified by column chromatography (silica mesh 100-200 at eluent of 10-20% ethyl acetate and hexane) to give 2-allyl-6-((1-methyl-1H-indazol-5-yl)amino)-1-(3-nitrophenyl)-1,2-dihydro-3H-pyrazolo[3,4-d]pyrimidin-3-one (600 mg, 1.221 mmol, 66.51% yield) as a yellow solid.

### tert-butyl(2-allyl-1-(3-nitrophenyl)-3-oxo-2,3-dihydro-1H-pyrazolo[3,4-d]pyrimidin-6-yl)(1-methyl-1H-indazol-5-yl) carbamate

To a stirred solution of 2-allyl-6-((1-methyl-1H-indazol-5-yl)amino)-1-(3-nitrophenyl)-1,2-dihydro-3H-pyrazolo[3,4-d]pyrimidin-3-one (0.2 g, 0.452 mmol) in DCM (5 mL) was added TEA (0.452 mmol) and Boc anhydride (0.148 g, 0.678 mmol). The mixture was stirred at under inert atmosphere for 16 h at room temperature. The progress of the reaction was monitored by TLC and UPLC. After completion of the reaction, the reaction mixture was diluted with water (100 mL) and extracted with ethyl acetate (2×200 mL). The combined organic layers were washed with brine (100 mL), dried over anhydrous sodium sulphate, filtered and concentrated under reduced pressure to give the compound tert-butyl (2-allyl-1-(3-nitrophenyl)-3-oxo-2,3-dihydro-1H-pyrazolo[3,4-d]pyrimidin-6-yl)(1-methyl-1H-indazol-5-yl) carbamate (250 mg, 0.424 mmol, 94% yield) as a white solid. LCMS m/z found: 443.4 (M−100).

### tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-nitrophenyl)-3-oxo-2,3-dihydro-1H-pyrazolo[3,4-d]pyrimidin-6-yl)(1-methyl-1H-indazol-5-yl) carbamate (250 mg, 0.461 mmol) in EtOH (3.0 mL) and water (10 mL) was added Iron (257 mg, 4.61 mmol) and NH₄Cl (246 mg, 4.61 mmol) and the mixture was allowed to stir for 16 h. The progress of the reaction was monitored by TLC and UPLC. After completion of the reaction, the reaction mixture was diluted with water (100 mL) and extracted with ethyl acetate (2×30 mL). The combined organic layers were washed with brine, dried over anhydrous Na₂SO₄, filtered and concentrated under reduced pressure to give the crude compound which was purified by column chromatography (silica gel mesh 100-200, at eluent of 50-80% ethyl acetate in hexane) to give tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate (0.150 g, 0.234 mmol, 50.8% yield) as a brown solid compound. LCMS m/z found: 513.4 (M+H).

### tert-butyl-(2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate (0.15 g, 0.293 mmol), 1-methylpiperidin-4-one (0.033 g, 0.293 mmol) in dichloroethane (5 mL) was added AcOH (1 mL) under inert atmosphere. The mixture was stirred for 4 h at room temperature and then cooled to 0 °C. STAB (0.311 g, 0.293 mmol) was added and the mixture was allowed to stir for 16 h. The progress of the reaction was monitored by TLC and UPLC. After completion of the reaction, the reaction mixture was diluted with water (100 mL) and extracted with ethyl acetate (2×100 mL). The combined organic layers were washed with brine (100 mL), dried over anhydrous sodium sulphate, filtered, and concentrated under reduced pressure to get the crude compound which was purified by flash column chromatography (silica mesh 100-200 at eluent of 0-20% MeOH and DCM) to give tert-butyl (2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (140 mg, 0.204 mmol, 69.8% yield) as a yellow solid compound.

### tert-butyl-(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (0.130 g, 0.213 mmol) in THF (2 mL) was added NaH (0.012 g, 0.533 mmol) and the mixture was allowed to stir for 30 min at 0 °C under inert atmosphere. Then was added Mel (0.030 g, 0.213 mmol) and the mixture was allowed to stir for 16 h at room temperature. The progress of the reaction was monitored by TLC and UPLC. After completion of the reaction, the reaction mixture was diluted with water (100 mL) and extracted with DCM (2×100 mL). The combined organic layers were washed with brine (100 mL), dried over anhydrous sodium sulphate, filtered, and concentrated under reduced pressure to give the crude compound tert-butyl(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (120 mg) as a yellow solid. The crude compound was taken as such for next step without purification.

### 2-allyl-1-(3-(methyl(1-methylpiperidin-4-yl)amino)phenyl)-6-((1-methyl-1H-indazol-5-yl)amino)-1,2-dihydro-3H-pyrazolo[3,4-d]pyrimidin-3-one (Compound 225)

To a stirred solution of tert-butyl(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (90 mg, 0.144 mmol) in DCM (5 mL) was added 4M HCl in 1,4dioxane (0.3 mL, 1.200 mmol) and the mixture was stirred at room temperature for 16 h. The progress of the reaction was monitored by TLC and UPLC. After completion of the reaction, the reaction mixture was concentrated under reduced pressure to get the crude compound which was purified by prep-HPLC (Column: X-select CSH C18, Mobile Phase A: 0.1% Formic acid in water, Mobile Phase B: Acetonitrile, Flowrate: 15.0 mL/Min, Rt-10.8) to give 2-allyl-1-(3-(methyl(1-methylpiperidin-4-yl)amino)phenyl)-6-((1-methyl-1H-indazol-5-yl)amino)-1,2-dihydro-3H-pyrazolo[3,4-d]pyrimidin-3-one (7.7 mg, 0.015 mmol, 10.09% yield) as an off-white solid.

### Example 65. Synthesis of 2-allyl-6-(1-methyl-1H-indazol-5-ylamino)-1-[m-(1-methyl-4-piperidyloxy)phenyl]-1,2-dihydro-3H-1,2,5,7-tetraazainden-3-one (Compound 220)

To a stirred solution of 2-allyl-6-((1-methyl-1H-indazol-5-yl)amino)-1-(3-(piperidin-4-yloxy)phenyl)-1,2-dihydro-3H-pyrazolo[3,4-d]pyrimidin-3-one (210 mg, 0.423 mmol) in THF (5 mL) was added 37% aq formaldehyde (172 mg, 2.114 mmol) at 25 °C, and resulting mixture was stirred for 10 min. Then STAB (269 mg, 1.269 mmol) was added portion wise. After complete addition of STAB, the reaction mixture was stirred at 25 °C for 16 h. The progress of reaction was monitored by UPLC. After completion of the reaction, the reaction mixture was quenched with TFA followed by ammonia in MeOH. The reaction mixture was diluted with water and extracted with 10% MeOH in DCM. The combined organic extract was washed with aq. sodium bicarbonate, dried over anhydrous Na₂SO₄, filtered and concentrated under reduced pressure to afford crude compound which was purified by Prep-HPLC to give 2-allyl-6-((1-methyl-1H-indazol-5-yl)amino)-1-(3-((1-methylpiperidin-4-yl)oxy)phenyl)-1,2-dihydro-3H-pyrazolo[3,4-d]pyrimidin-3-one (21.22 mg, 0.040 mmol, 9.53% yield) as a white solid.

### Example 66. Synthesis of p-{2-ethyl-1-[6-(1-methyl-4-piperidyloxy)-2-pyridyl]-3-oxo-1,2-dihydro-3H-1,2,5,7-tetraazainden-6-ylamino}benzonitrile (Compound 310)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 37.1 mg (44%).

### Example 67. Synthesis of 2-allyl-6-(1-benzofuran-5-ylamino)-1-[6-(1-methyl-4-piperidyloxy)-2-pyridyl]-1,2-dihydro-3H-1,2,5,7-tetraazainden-3-one (Compound 309)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 154 mg TFA salt (84%) as a pale-yellow powder.

### Example 68. Synthesis of 2-allyl-6-(1-benzofuran-6-ylamino)-1-[6-(1-methyl-4-piperidyloxy)-2-pyridyl]-1,2-dihydro-3H-1,2,5,7-tetraazainden-3-one (Compound 308)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 114 mg TFA salt (62%) as a pale-yellow powder.

### Example 69. Synthesis of 2-allyl-6-(2H-1,3-benzodioxol-5-ylamino)-1-[6-(1-methyl-4-piperidyloxy)-2-pyridyl]-1,2-dihydro-3H-1,2,5,7-tetraazainden-3-one (Compound 307)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 130 mg TFA salt (70%) as a pale-yellow powder.

### Example 70. Synthesis of m-{2-allyl-1-[6-(1-methyl-4-piperidyloxy)-2-pyridyl]-3-oxo-1,2-dihydro-3H-1,2,5,7-tetraazainden-6-ylamino}benzonitrile (Compound 305)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 25 mg (27%).

### Example 71. Synthesis of 2-allyl-6-(1,3-benzothiazol-6-ylamino)-1-[6-(1-methyl-4-piperidyloxy)-2-pyridyl]-1,2-dihydro-3H-1,2,5,7-tetraazainden-3-one (Compound 303)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 19 mg TFA salt (80%) as a pale-yellow powder.

### Example 72. Synthesis of p-{2-ethyl-3-oxo-1-[6-(4-piperidyloxy)-2-pyridyl]-1,2-dihydro-3H-1,2,5,7-tetraazainden-6-ylamino}benzonitrile (Compound 302)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using p-bromobenzonitrile and tert-butyl 4-[(6-{6-amino-2-ethyl-3-oxo-1H,2H,3H-pyrazolo[3,4-d]pyrimidin-1-yl}pyridin-2-yl)oxy]piperidine-1-carboxylate. Yield: 7.8 mg (9%).

### Example 73. Synthesis of p-{2-allyl-1-[6-(1-methyl-4-piperidyloxy)-2-pyridyl]-3-oxo-1,2-dihydro-3H-1,2,5,7-tetraazainden-6-ylamino}benzonitrile (Compound 301)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using p-bromobenzonitrile.

### Example 74. p-{2-allyl-3-oxo-1-[6-(4-piperidyloxy)-2-pyridyl]-1,2-dihydro-3H-1,2,5,7-tetraazainden-6-ylamino}benzonitrile (Compound 300)

### 4-{6-[2-allyl-6-(p-cyanophenylamino)-3-oxo-1,2-dihydro-3H-1,2,5,7-tetraazainden-1-yl]-2-pyridyloxy}-1-piperidinecarboxylate

tert-butyl-4-[6-(2-allyl-6-amino-3-oxo-1,2-dihydro-3H-1,2,5,7-tetraazainden-1-yl)-2-pyridyloxy]-1-piperidinecarboxylate (200 mg, 0.428 mmol), p-bromobenzonitrile (155.8 mg, 0.856 mmol), Cs₂CO₃ (418 mg, 3 equiv) were mixed and stirred under nitrogen in dioxane (5 mL) for 15 min. A catalytic amount of CuI (252.6 mg, 3.1 eq), and 1,2-bis(methylamino) ethane as a ligand (41.5 mg, 0.471 mmol) were added. The reaction mixture was heated up to 90 °C for 48 h. The progress of the reaction was observed by LCMS. The reaction mixture was cooled to rt, and the crude residue was quenched with saturated aqueous ammonia solution, then extracted with EtOAc (3×10 mL) and washed with brine. The combined organic layers were dried (anhyd. Na₂SO₄) and the solvent was evaporated under reduced pressure. The crude product was dissolved in MeCN (5 ml) and the product made to crystalize over 1 h by adding slowly water (5 ml). The pale-brown solid crude material was dissolved in DCM (5 ml), loaded onto a 10 g silica column and purified by flash chromatography (0-100%, EtOAc:PE) to give tert-butyl 4-{6-[2-allyl-6-(p-cyanophenylamino)-3-oxo-1,2-dihydro-3H-1,2,5,7-tetraazainden-1-yl]-2-pyridyloxy}-1-piperidinecarboxylate [175 mg, yield 72%] as a yellow solid.

### p-{2-allyl-3-oxo-1-[6-(4-piperidyloxy)-2-pyridyl]-1,2-dihydro-3H-1,2,5,7-tetraazainden-6-ylamino}benzonitrile as a trifluoroacetic acid salt (Compound 300)

A solution of tert-butyl 4-{6-[2-allyl-6-(p-cyanophenylamino)-3-oxo-1,2-dihydro-3H-1,2,5,7-tetraazainden-1-yl]-2-pyridyloxy}-1-piperidinecarboxylate (175 mg, 0.308 mmol) in DCM (3 mL) was treated with TFA (1.5 mL) for 2 h. The solvent was removed in vacuo and the crude was dissolved in EtOAc, washed with sat aq NaHCO₃, brine, dried (MgSO₄), and concentrated. The resulting material was purified by reverse phase chromatography (Gemini NX—C18, 21*150 mm, water (0.1% TFA)/acetonitrile, gradient over 12 minutes, 25 ml/min). The pure fractions were pooled and concentrated to give p-{2-allyl-3-oxo-1-[6-(4-piperidyloxy)-2-pyridyl]-1,2-dihydro-3H-1,2,5,7-tetraazainden-6-ylamino}benzonitrile as a trifluoroacetic acid salt as a white solid [165 mg].

### Example 75. 2-allyl-6-(1,2-benzisoxazol-6-ylamino)-1-[6-(1-methyl-4-piperidyloxy)-2-pyridyl]-1,2-dihydro-3H-1,2,5,7-tetraazainden-3-one (Compound 299)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 55 mg (42%) as a white powder.

### Example 76. 2-allyl-6-(1-methyl-1H-indazol-5-ylamino)-1-[m-(4-piperidyloxy)phenyl]-1,2-dihydro-3H-1,2,5,7-tetraazainden-3-one (Compound 223)

### tert-butyl-4-(3-bromophenoxy)piperidine-1-carboxylate

To a stirred solution of tert-butyl 4-hydroxypiperidine-1-carboxylate (7.48 g, 37.1 mmol) in DMF (50 mL) was added NaH (2.285 g, 57.1 mmol) at 0 °C under inert atmosphere, and then this mixture was stirred for 1 h at 50 °C. The reaction mixture was cooled to room temperature and a solution of 1-bromo-3-fluorobenzene (5 g, 28.6 mmol) dissolved in DMF (5 mL) was added. After addition, the reaction mixture was stirred at 70 °C for 3 h. The progress of the reaction was monitored by LCMS and TLC. After completion of the reaction, the reaction mixture was quenched with ice cold water and extracted with ethyl acetate (2×400 mL). The organic phases were combined and washed with brine (500 mL) and dried over anhydrous Na₂SO₄, filtered and concentrated under reduced pressure to get a crude compound which was purified by column chromatography (silica mesh 100-200 at eluent of 10-20% ethyl acetate and hexane) to give tert-butyl 4-(3-bromophenoxy)piperidine-1-carboxylate (6.8 g, 14.89 mmol, 52.1% yield) as yellow gummy liquid. The isolated product was taken as such for next step.

### tert-butyl-4-(3-(2-allyl-6-(methylthio)-3-oxo-2,3-dihydro-1H-pyrazolo[3,4-d]pyrimidin-1-yl)phenoxy)piperidine-1-carboxylate

A stirred solution of tert-butyl 4-(3-bromophenoxy)piperidine-1-carboxylate (481 mg, 1.350 mmol), 2-allyl-6-(methylthio)-1,2-dihydro-3H-pyrazolo[3,4-d]pyrimidin-3-one (250 mg, 1.125 mmol), K₂CO₃ (466 mg, 3.37 mmol) and N,N′-dimethylethylenediamine (99 mg, 1.125 mmol) in dioxane (10 mL) was degassed for 20 min at room temperature under inert atmosphere. Then CuI (214 mg, 1.125 mmol) was added and the mixture again degassed for 5 min. The reaction mixture was stirred at 100 °C for 16 h. The progress of the reaction was monitored by TLC and LCMS. After completion of the reaction, the reaction mixture was diluted with DCM and filtered through celite pad. The collected organic fraction was concentrated under reduced pressure to get the crude compound which was purified by flash column chromatography (SiO₂/230-400 mesh; 20-50% ethyl acetate-pet ether) to give tert-butyl-4-(3-(2-allyl-6-(methylthio)-3-oxo-2,3-dihydro-1H-pyrazolo[3,4-d]pyrimidin-1-yl)phenoxy)piperidine-1-carboxylate (80 mg, 0.154 mmol, 13.72% yield) as an off-white solid.

### tert-butyl-4-(3-(2-allyl-6-(methylsulfonyl)-3-oxo-2,3-dihydro-1H-pyrazolo[3,4-d]pyrimidin-1-yl)phenoxy)piperidine-1-carboxylate

To the stirred solution of tert-butyl 4-(3-(2-allyl-6-(methylthio)-3-oxo-2,3-dihydro-1H-pyrazolo[3,4-d]pyrimidin-1yl)phenoxy)piperidine-1-carboxylate (80 mg, 0.161 mmol) in DCM (5 mL) was added m-CPBA (55.5 mg, 0.322 mmol) at room temperature and the mixture was stirred for 2 h. The progress of the reaction was monitored by TLC and LCMS. After completion of the reaction, it was quenched the with sodium bicarbonate solution and then extracted with 10% MeOH in DCM (2×50 mL). The combined organic layers was dried over sodium sulphate and evaporated under reduced pressure to give tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (85 mg, 0.053 mmol, 32.9% yield). This compound was taken as such for the next step without further purification.

### tert-butyl-4-(3-(2-allyl-6-((1-methyl-1H-indazol-5-yl)amino)-3-oxo-2,3-dihydro-1H-pyrazolo[3,4-d]pyrimidin-1-yl)phenoxy)piperidine-1-carboxylate

To the stirred solution of tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (65 mg, 0.123 mmol) in AcOH (3 mL) was added 1-methyl-1H-indazol-5-amine (18.06 mg, 0.123 mmol) at room temperature. The reaction mixture was allowed to stir for 16 h. The progress of the reaction was monitored by TLC and LCMS. After completion of the reaction, the reaction mass was quenched with aq. sodium bicarbonate solution and extracted with 10% MeOH in DCM (2×100 mL). The combined organic layer was dried over Na₂SO₄, then concentrated under reduced pressure to give crude compound which was purified by column chromatography (SiO₂/230-400 mesh; 0-20% MeOH-DCM) to give tert-butyl 4-(3-(2-allyl-6-((1-methyl-1H-indazol-5-yl)amino)-3-oxo-2,3-dihydro-1H-pyrazolo[3,4-d]pyrimidin-1-yl)phenoxy)piperidine-1-carboxylate (30 mg). The isolated product was taken as such for next step.

### 2-allyl-6-((1-methyl-1H-indazol-5-yl)amino)-1-(3-(piperidin-4-yloxy)phenyl)-1,2-dihydro-3H-pyrazolo[3,4-d]pyrimidin-3-one (Compound 223)

To a stirred solution of tert-butyl 4-(3-(2-allyl-6-((1-methyl-1H-indazol-5-yl)amino)-3-oxo-2,3-dihydro-1H-pyrazolo[3,4-d]pyrimidin-1-yl)phenoxy)piperidine-1-carboxylate (30 mg, 0.050 mmol) in dioxane (1 mL) was added 4M HCl in Dioxane (0.2 ml) at 0 °C under inert atmosphere, and the mixture then stirred for 16 h. The progress of the reaction was monitored by TLC and LCMS. After completion of the reaction, the solvent was evaporated under reduced pressure and washed with n-hexane (2×5 mL) and then purified by prep HPLC (Column: X-select CSH C18, Mobile phase A: Water (with 0.1% AA), Mobile phase B: Acetonitrile, Flow rate-15.0 mL/Min, Rt-12.8) to give 2-allyl-6-((1-methyl-1H-indazol-5-yl)amino)-1-(3-(piperidin-4-yloxy)phenyl)-1,2-dihydro-3H-pyrazolo[3,4-d]pyrimidin-3-one (4 mg, 7.97 μmol, 15.86% yield) as a brown solid.

### Example 77. 2-allyl-6-(1-methyl-1H-indazol-5-ylamino)-1-[m-(1-methyl-4-piperidylamino)phenyl]-1,2-dihydro-3H-1,2,5,7-tetraazainden-3-one (Compound 226)

### 2-allyl-6-(methylthio)-1-(3-nitrophenyl)-1,2-dihydro-3H-pyrazolo[3,4-d]pyrimidin-3-one

To a stirred solution of 2-allyl-6-(methylthio)-1,2-dihydro-3H-pyrazolo[3,4-d]pyrimidin-3-one (1, 1.2 g, 5.40 mmol) in dichloroethane (15 mL) was added 3-nitrophenyl boronic acid (1.352 g, 8.10 mmol), sodium carbonate (1.707 g, 16.20 mmol) and copper(II) acetate (0.490 g, 2.70 mmol) followed by pyridine (0.169 g, 1.080 mmol) at room temperature and the temperature was raised to 70 °C and stirred for 16 h. The progress of the reaction was monitored by TLC and LCMS. After completion of the reaction, the reaction mixture was filtered through celite and extracted with 10% MeOH in DCM (2×100 mL). The combined organic layers were dried over anhydrous Na₂SO₄ and then evaporated under reduced pressure to give the crude compound which was purified by column chromatography (silica mesh 100-200 at eluent of 70-100% ethyl acetate and hexane) to give 2-allyl-6-(methylthio)-1-(3-nitrophenyl)-1,2-dihydro-3H-pyrazolo[3,4-d]pyrimidin-3-one (700 mg, 1.794 mmol, 33.2% yield) as a white solid.

### 2-allyl-6-(methylsulfonyl)-1-(3-nitrophenyl)-1,2-dihydro-3H-pyrazolo[3,4-d]pyrimidin-3-one

To a stirred solution of 2-allyl-6-(methylthio)-1-(3-nitrophenyl)-1,2-dihydro-3H-pyrazolo[3,4-d]pyrimidin-3-one (50 mg, 0.146 mmol) in DCM (10 ml) was added mCPBA (53.7 mg, 0.218 mmol) at room temperature under inert atmosphere, and then continued stirring for 2 h. The progress of the reaction was monitored by TLC and UPLC. After completion of the reaction, the reaction mixture was quenched with aq. 10% sodium bicarbonate (10 mL) and extracted with 10% MeOH in DCM (2×50 mL). The combined organic layers were washed with brine (100 mL), dried over anhydrous sodium sulphate, filtered and concentrated under reduced pressure to give the crude compound 2-allyl-6-(methylsulfonyl)-1-(3-nitrophenyl)-1,2-dihydro-3H-pyrazolo[3,4-d]pyrimidin-3-one (30 mg, 0.024 mmol, 16.47% yield) as an off-white solid. This crude compound as such taken for the next step without any further purification.

### 2-allyl-6-((1-methyl-1H-indazol-5-yl)amino)-1-(3-nitrophenyl)-1,2-dihydro-3H-pyrazolo[3,4-d]pyrimidin-3-one

To a stirred solution of 2-allyl-6-(methylthio)-1-(3-nitrophenyl)-1,2-dihydro-3H-pyrazolo[3,4-d]pyrimidin-3-one (0.7 g, 2.039 mmol) in AcOH (5 mL) was added 1-methyl-1H-indazol-5-amine (0.3 g, 2.039 mmol) and allowed to stirred for 16 h at room temperature. The progress of the reaction was monitored by TLC and UPLC. After completion of the reaction, the solvent was evaporated under reduced pressure and the residue quenched with aq. 10% sodium bicarbonate solution (100 mL). The mixture was extracted with ethyl acetate (2×300 mL). The combined organic extract was washed with brine (100 mL), dried over anhydrous sodium sulphate, filtered and concentrated under reduced pressure to give the crude compound which was purified by column chromatography (silica gel, mesh 100-200 at eluent of 10-20% ethyl acetate and hexane) to give 2-allyl-6-((1-methyl-1H-indazol-5-yl)amino)-1-(3-nitrophenyl)-1,2-dihydro-3H-pyrazolo[3,4-d]pyrimidin-3-one (600 mg, 1.221 mmol, 66.51% yield) as a yellow solid.

### 2-allyl-1-(3-aminophenyl)-6-((1-methyl-1H-indazol-5-yl)amino)-1,2-dihydro-3H-pyrazolo[3,4-d]pyrimidin-3-one

To a stirred solution of 2-allyl-6-((1-methyl-1H-indazol-5-yl)amino)-1-(3-nitrophenyl)-1,2-dihydro-3H-pyrazolo[3,4-d]pyrimidin-3-one (50 mg, 0.113 mmol) in EtOH (3.0 mL) and water (10 mL) was added iron (63.1 mg, 1.130 mmol) and NH₄Cl (60.4 mg, 1.130 mmol) and the temperature was raised to 60 °C and the mixture stirred for 2 h. The progress of the reaction was monitored by TLC and UPLC. After completion of the reaction, the reaction mixture was diluted with water (100 mL) and extracted with ethyl acetate (2×300 mL). The combined organic layers were washed with brine (100 mL), dried over anhydrous sodium sulphate, filtered and concentrated under reduced pressure to give the crude compound which was purified by flash column chromatography (silica mesh 100-200 at eluent of 50-80% ethyl acetate and hexane) to give 2-allyl-1-(3-aminophenyl)-6-((1-methyl-1H-indazol-5-yl)amino)-1,2-dihydro-3H-pyrazolo[3,4-d]pyrimidin-3-one (200 mg, 0.023 mmol, 20.38% yield) as a white solid compound.

### 2-allyl-6-((1-methyl-1H-indazol-5-yl)amino)-1-(3-((1-methylpiperidin-4-yl)amino)phenyl)-1,2-dihydro-3H-pyrazolo[3,4-d]pyrimidin-3-one (Compound 226)

To a stirred solution of 2-allyl-1-(3-aminophenyl)-6-((1-methyl-1H-indazol-5-yl)amino)-1,2-dihydro-3H-pyrazolo[3,4-d]pyrimidin-3-one (0.2 g, 0.485 mmol) and 1-methylpiperidin-4-one (55 mg, 0.485 mmol) was added AcOH (1 mL) at room temperature and the resulting mixture was allowed to stir for 2 h. Then it was cooled to 0 °C and STAB (0.617 g, 2.91 mmol) was added and the mixture was allowed to stir at room temperature for 16 h. The progress of the reaction was monitored by TLC and LCMS. After completion of the reaction, the reaction mixture was quenched with ice cold water and extracted with DCM (2×100 mL). The combined organic layers were dried over anhydrous Na₂SO₄, and evaporated under reduced pressure to give crude compound which was purified by prep HPLC (Column: X-select CSH C18, Mobile Phase A: 0.1% formic acid in water, Mobile Phase B: acetonitrile, Flowrate: 15.0 mL/Min, Rt-10.8) to give 2-allyl-6-((1-methyl-1H-indazol-5-yl)amino)-1-(3-((1-methylpiperidin-4-yl)amino)phenyl)-1,2-dihydro-3H-pyrazolo[3,4-d]pyrimidin-3-one (14 mg, 0.026 mmol, 5.44% yield) as an off-white solid.

### Example 78. 2-ethyl-1-[6-(4-piperidyloxy)-2-pyridyl]-6-m-toluidino-1,2-dihydro-3H-1,2,5,7-tetraazainden-3-one (Compound 298)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 55 mg (42%) as a white powder. Yield: 92.5 mg, 80% as off-white solid.

### Example 79. 2-ethyl-6-(p-fluorophenylamino)-1-[6-(4-piperidyloxy)-2-pyridyl]-1,2-dihydro-3H-1,2,5,7-tetraazainden-3-one (Compound 297)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 55 mg (42%) as a white powder. Yield: 50.0 mg, 43% as off-white solids.

### Example 80. 2-allyl-6-(1-methyl-4-pyrazolylamino)-1-[m-(4-piperidyloxy)phenyl]-1,2-dihydro-3H-1,2,5,7-tetraazainden-3-one (Compound 224)

### tert-butyl-4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate

To a stirred solution of tert-butyl 4-(3-(2-allyl-6-(methylsulfonyl)-3-oxo-2,3-dihydro-1H-pyrazolo[3,4-d]pyrimidin-1-yl)phenoxy)piperidine-1-carboxylate (80 mg, 0.151 mmol) in acetic acid (3 mL) was added 1-methyl-1H-pyrazol-4-amine (14.67 mg, 0.151 mmol) at 25 °C. The reaction mixture was allowed to stir at 25 °C for 16 h. The progress of the reaction was monitored by TLC and LCMS. After completion of the reaction. The reaction mixture was concentrated under reduced pressure and then diluted with 10% aq. sodium bicarbonate and then extracted with 10% MeOH in DCM. The combined organic layers were dried over anhydrous sodium sulphate, filtered and concentrated under reduced pressure to give crude compound which was purified using column chromatography (silica gel, mesh 100-200 at eluent of 5-10% MeOH in DCM) to give tert-butyl 4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate (60 mg, 0.082 mmol, 54.5% yield) as an off-white solid.

### 2-allyl-6-((1-methyl-1H-pyrazol-4-yl)amino)-1-(3-(piperidin-4-yloxy)phenyl)-1,2-dihydro-3H-pyrazolo[3,4-d]pyrimidin-3-one (Compound 224)

To a stirred solution of tert-butyl 4-(3-(2-allyl-6-((1-methyl-1H-pyrazol-4-yl)amino)-3-oxo-2,3-dihydro-1H-pyrazolo[3,4-d]pyrimidin-1-yl)phenoxy)piperidine-1-carboxylate (60 mg, 0.110 mmol) in 1,4-dioxane (5 ml) was added 4M HCl (0.329 mL, 1.317 mmol) in dioxane at 0 °C under inert atmosphere. The reaction was allowed to stir at room temperature for 8 h. The progress of the reaction was monitored by TLC and LCMS. After completion of the reaction, the reaction was concentrated under reduced pressure to give the crude which was purified by Prep-HPLC (Column: X-select CSH C18, Mobile phase A: water (with 0.1% FA), Mobile phase B: acetonitrile (with 0.1% FA), Flow rate-15.0 mL/Min, Rt-12.8) to give 2-allyl-6-((1-methyl-1H-pyrazol-4-yl)amino)-1-(3-(piperidin-4-yloxy)phenyl)-1,2-dihydro-3H-pyrazolo[3,4-d]pyrimidin-3-one (8 mg, 0.018 mmol, 16% yield) as a brown solid.

### Example 81. 2-allyl-1-[6-(4-piperidyloxy)-2-pyridyl]-6-[p-(2,2,2-trifluoroethoxy)phenylamino]-1,2-dihydro-3H-1,2,5,7-tetraazainden-3-one (Compound 227)

### tert-butyl-4-(6-{2-allyl-3-oxo-6-[p-(2,2,2-trifluoroethoxy)phenylamino]-1,2-dihydro-3H-1,2,5,7-tetraazainden-1-yl}-2-pyridyloxy)-1-piperidinecarboxylate

mCPBA (<77% pure) (166 mg, assumed 0.722 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (300 mg, 0.602 mmol) in DCM (2.5 mL) at room temperature under nitrogen. After 15 min, DCM was concentrated in vacuo, and the crude product solubilized in MeCN (3 ml). Then p-(2,2,2-trifluoroethoxy) aniline (115 mg, 0.602 mmol) was added and the reaction mixture stirred at 60 °C in a closed vial. After 18 h, the reaction mixture was allowed to cool to RT, and mCPBA quenched with 1M NaOH (5 mL) which was added dropwise. The aqueous phase was extracted with EtOAc (3×20 mL) and washed with brine (20 mL). The combined organic layers were dried (MgSO₄) and concentrated under reduced pressure to give a yellow powder. The crude material was dissolved in DCM (5 ml), loaded onto a 10 g silica column and purified by flash chromatography (0-100%, EtOAc:PE) to give tert-butyl 4-(6-{2-allyl-3-oxo-6-[p-(2,2,2-trifluoroethoxy)phenylamino]-1,2-dihydro-3H-1,2,5,7-tetraazainden-1-yl}-2-pyridyloxy)-1-piperidinecarboxylate [210 mg, 94%] as a pale-yellow solid.

### 2-allyl-6-[p-(2-fluoroethoxy)phenylamino]-1-[6-(4-piperidyloxy)-2-pyridyl]-1,2-dihydro-3H-1,2,5,7-tetraazainden-3-one trifluoroacetic acid salt (Compound 227)

A solution of tert-butyl 4-(6-{2-allyl-3-oxo-6-[p-(2,2,2-trifluoroethoxy)phenylamino]-1,2-dihydro-3H-1,2,5,7-tetraazainden-1-yl}-2-pyridyloxy)-1-piperidinecarboxylate (210 mg, 0.360 mmol) in DCM (5 mL) was treated with TFA (2.5 mL) for 2 h. The solvent was removed in vacuo and the crude was dissolved in EtOAc, washed with sat aq NaHCO₃, brine, dried (MgSO₄), and concentrated. The resulting material was purified by reversed phase chromatography (Gemini NX—C18, 21*150 mm, water (0.1% TFA)/acetonitrile, gradient over 12 minutes, 25 ml/min). The pure fractions were pooled and concentrated to give 2-allyl-6-[p-(2-fluoroethoxy)phenylamino]-1-[6-(4-piperidyloxy)-2-pyridyl]-1,2-dihydro-3H-1,2,5,7-tetraazainden-3-one as a trifluoroacetic acid salt as a white solid. [1.4 mg].

### Example 82. 5-{2-allyl-3-oxo-1-[6-(4-piperidyloxy)-2-pyridyl]-1,2-dihydro-3H-1,2,5,7-tetraazainden-6-ylamino}-2-toluonitrile (Compound 293)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield: 27 mg, 47%.

### Example 83. 2-ethyl-6-(p-fluorophenylamino)-1-[6-(1-methyl-4-piperidyloxy)-2-pyridyl]-1,2-dihydro-3H-1,2,5,7-tetraazainden-3-one (Compound 290)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 55 mg (42%) as a white powder. Yield: 156 mg, 43% as yellow solid.

### Example 84. 2-ethyl-1-[6-(1-methyl-4-piperidyloxy)-2-pyridyl]-6-m-toluidino-1,2-dihydro-3H-1,2,5,7-tetraazainden-3-one (Compound 289)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 55 mg (42%) as a white powder. Yield: 82 mg, 53% as off-white solid.

### Example 85. 2-allyl-1-(6-(methyl(1-methylpiperidin-4-yl)amino)pyridin-2-yl)-6-((1-(3,3,3-trifluoropropyl)-1H-pyrazol-4-yl)amino)-1,2-dihydro-3H-pyrazolo[3,4-d]pyrimidin-3-one (Compound 201)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 28 mg (25.2%).

### Example 86. 2-allyl-6-(1-isobutyl-4-pyrazolylamino)-1-[6-(1-methyl-4-piperidyloxy)-2-pyridyl]-1,2-dihydro-3H-1,2,5,7-tetraazainden-3-one (Compound 200)

### tert-butyl 4-{6-[6-(1-isobutylpyrazole-4-ylamino)-3-oxo-2-(prop-2-enyl)-1,2-dihydro-3H-1,2,5,7-tetraazainden-1-yl]pyrid-2-yloxy}piperidine-1-carboxylate

mCPBA (<77% pure) (108 mg, assumed 0.469 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (123 mg, 0.247 mmol) in DCM (5 mL) at room temperature under nitrogen. After 15 min, DCM was concentrated in vacuo, and the crude solubilized in MeCN (5 ml). Then 1-isobutyl-4-pyrazolylamine (54.4 mg, 0.391 mmol) was added. The reaction mixture was stirred at 60 °C in a closed vial. After 22 h, the reaction mixture was allowed to cool to RT, and mCPBA quenched with 1M NaOH (5 mL) which was added dropwise. The aqueous phase was extracted with EtOAc (3×20 mL). The combined organic layers were washed with brine and dried (MgSO₄) and concentrated under reduced pressure to give the crude product as a yellow powder. The crude material was dissolved in DCM (5 ml), loaded onto a 10 g silica column and purified by flash chromatography (0-100%, EtOAc:PE) to give tert-butyl 4-{6-[6-(1-isobutylpyrazole-4-ylamino)-3-oxo-2-(prop-2-enyl)-1,2-dihydro-3H-1,2,5,7-tetraazainden-1-yl]pyrid-2-yloxy}piperidine-1-carboxylate as a pale-yellow solid (100 mg, 84%).

### 2-allyl-6-(1-isobutyl-4-pyrazolylamino)-1-[6-(4-piperidyloxy)-2-pyridyl]-1,2-dihydro-3H-1,2,5,7-tetraazainden-3-one

A solution of tert-butyl 4-{6-[6-(1-isobutylpyrazol-4-ylamino)-3-oxo-2-(prop-2-enyl)-1,2-dihydro-3H-1,2,5,7-tetraazainden-1-yl]pyrid-2-yloxy}piperidine-1-carboxylate (100 mg, 0.205 mmol) in DCM (5 mL) was treated with TFA (2 mL) for 2 h. The solvent was removed in vacuo and the crude was dissolved in EtOAc, washed with sat aq NaHCO₃, brine, dried (MgSO₄), and concentrated. The resulting material was purified by HPLC (Phenomenex Gemini 5 μm NX—C18 110 Å 150×21, 2 mm, buffer 0.2% NH₄OH, water (0.2% NH₄OH)/acetonitrile, gradient over 9 minutes, 30 ml/min). The pure fractions were pooled and concentrated to give 2-allyl-6-(1-isobutyl-4-pyrazolylamino)-1-[6-(4-piperidyloxy)-2-pyridyl]-1,2-dihydro-3H-1,2,5,7-tetraazainden-3-one as a white solid (100 mg).

### 2-allyl-6-(1-isobutyl-4-pyrazolylamino)-1-[6-(1-methyl-4-piperidyloxy)-2-pyridyl]-1,2-dihydro-3H-1,2,5,7-tetraazainden-3-one (Compound 200)

2-allyl-6-(1-isobutyl-4-pyrazolylamino)-1-[6-(4-piperidyloxy)-2-pyridyl]-1,2-dihydro-3H-1,2,5,7-tetraazainden-3-one (100 mg, 0.205 mmol) was dissolved in anhydrous THF (5 mL). Formaldehyde (~36% pure, 30.4 μL, 0.408 mmol) and STAB (86.5 mg, 0.408 mmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with EtOAc (3×20 mL). The combined organic layers were dried (MgSO₄) and concentrated under reduced pressure to give the crude material as a yellow powder. The crude product was dissolved in MeOH (5 ml) and was purified by HPLC (Phenomenex Gemini 5 μm NX—C18 110 Å 150×21, 2 mm, buffer 0.2% NH₄OH, water (0.2% NH₄OH)/acetonitrile, gradient over 6 minutes, 30 ml/min). The pure fractions were pooled and concentrated to give 2-allyl-6-(1-isobutyl-4-pyrazolylamino)-1-[6-(1-methyl-4-piperidyloxy)-2-pyridyl]-1,2-dihydro-3H-1,2,5,7-tetraazainden-3-one (60 mg, 60%).

### Example 87. 2-allyl-6-(1-isopropyl-4-pyrazolylamino)-1-[6-(1-methyl-4-piperidyloxy)-2-pyridyl]-1,2-dihydro-3H-1,2,5,7-tetraazainden-3-one (Compound 198)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 60 mg (24%).

### Example 88. 2-allyl-1-[6-(1-methyl-4-piperidyloxy)-2-pyridyl]-6-(6-methyl-3-pyridylamino)-1,2-dihydro-3H-1,2,5,7-tetraazainden-3-one (Compound 197)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 29 mg TFA salt (83%) as a pale-yellow powder.

### Example 89. 2-allyl-6-(2H-1,3-benzodioxol-5-ylamino)-1-[6-(1-methyl-4-piperidylamino)-2-pyridyl]-1,2-dihydro-3H-1,2,5,7-tetraazainden-3-one (Compound 196)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. The purified material was converted into the free base by solid-phase extraction (1 g SCX-2, MeOH, NH₃/MeOH 1.4 M). Yield: 41 mg free base (33%) as a brown solid.

### Example 90. 2-allyl-6-(1-benzofuran-6-ylamino)-1-[6-(1-methyl-4-piperidylamino)-2-pyridyl]-1,2-dihydro-3H-1,2,5,7-tetraazainden-3-one (Compound 195)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 110 mg TFA salt (74%) as a yellow solid.

### Example 91. 2-allyl-1-{6-(9-methyl-9-azabicyclo[3.3.1]non-3-yloxy)-2-pyridyl}-6-(1-methyl-4-pyrazolylamino)-1,2-dihydro-3H-1,2,5,7-tetraazainden-3-one (Compound 194)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 40 mg (31.7%).

### Example 92. 1-{6-[(R)-3-piperidyloxy]-2-pyridyl}-2-allyl-6-(p-chlorophenylamino)-1,2-dihydro-3H-1,2,5,7-tetraazainden-3-one (Compound 192)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one.

### Example 93. 2-allyl-1-[6-(1-methyl-4-piperidyloxy)-2-pyridyl]-6-m-toluidino-1,2-dihydro-3H-1,2,5,7-tetraazainden-3-one (Compound 191)

### tert-butyl 4-{6-[6-(3-methylphenylamino)-3-oxo-2-(prop-2-enyl)-1,2-dihydro-3H-1,2,5,7-tetraazainden-1-yl]pyrid-2-yloxy}piperidine-1-carboxylate

mCPBA (<77% pure) (167.2 mg, assumed 0.727 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (300 mg, 0.602 mmol) in DCM (5 mL) at room temperature under nitrogen. After 15 min, DCM was concentrated in vacuo, and the crude solubilized in MeCN (5 ml) then m-toluidine (96.7 mg, 0.903 mmol) was added. The reaction mixture stirred at 60 °C in a closed vial. After 22 h, the reaction mixture was allowed to cool to RT, and mCPBA was quenched with 1M NaOH (5 mL) which was added dropwise. The aqueous phase was extracted with EtOAc (3×20 mL). The combined organic layers were washed with brine (20 mL), then dried (MgSO₄) and concentrated under reduced pressure to give the product as a yellow powder. The crude material was dissolved in DCM (5 ml), loaded onto a 10 g silica column and purified by flash chromatography (0-100%, EtOAc: PE) to give tert-butyl 4-{6-[6-(3-methylphenylamino)-3-oxo-2-(prop-2-enyl)-1,2-dihydro-3H-1,2,5,7-tetraazainden-1-yl]pyrid-2-yloxy}piperidine-1-carboxylate [300 mg, 87.2%] as a pale-yellow solid.

### 2-allyl-1-[6-(4-piperidyloxy)-2-pyridyl]-6-m-toluidino-1,2-dihydro-3H-1,2,5,7-tetraazainden-3-one trifluoroacetic acid salt

A solution of tert-butyl 4-{6-[6-(3-methylphenylamino)-3-oxo-2-(prop-2-enyl)-1,2-dihydro-3H-1,2,5,7-tetraazainden-1-yl]pyrid-2-yloxy}piperidine-1-carboxylate (300 mg, 0.525 mmol) in DCM (5 mL) was treated with TFA (2 mL) for 2 h. The solvent was removed in vacuo and the crude was dissolved in EtOAc, washed with sat aq NaHCO₃, brine, dried (MgSO₄), and concentrated. The resulting material was purified by HPLC (Phenomenex Gemini 5 μm NX—C18 110 Å 150×21, 2 mm, buffer 0.2% NH₄OH, water (0.2% NH₄OH)/acetonitrile, gradient over 9 minutes, 30 ml/min). The pure fractions were pooled and concentrated to give 2-allyl-1-[6-(4-piperidyloxy)-2-pyridyl]-6-m-toluidino-1,2-dihydro-3H-1,2,5,7-tetraazainden-3-one as a trifluoroacetic acid salt as a white solid [120 mg].

### 2-allyl-1-[6-(1-methyl-4-piperidyloxy)-2-pyridyl]-6-m-toluidino-1,2-dihydro-3H-1,2,5,7-tetraazainden-3-one (Compound 191)

2-allyl-1-[6-(4-piperidyloxy)-2-pyridyl]-6-m-toluidino-1,2-dihydro-3H-1,2,5,7-tetraazainden-3-one (120 mg, 0.21 mmol) was dissolved in anhydrous THF (5 mL). Formaldehyde (~36% pure, 31 μL, 0.21 mmol) and STAB (89 mg, 0.21 mmol) were added in that order. The reaction mixture was stirred at room temperature while monitored by LCMS. After 2 h, the reaction was quenched with saturated aqueous sodium bicarbonate (5 mL) which was added dropwise. The aqueous phase was extracted with EtOAc (3×20 mL). The combined organic layers were dried (MgSO₄) and concentrated under reduced pressure to give the crude material as a yellow powder.

The crude product was dissolved in MeOH (5 ml) and was purified by HPLC (Phenomenex Gemini 5 μm NX—C18 110 Å 150×21, 2 mm, buffer 0.2% NH₄OH, water (0.2% NH₄OH)/acetonitrile, gradient over 6 minutes, 30 ml/min). The pure fractions were pooled and concentrated giving [100 mg, yield 33%] as a white solid giving 2-allyl-1-[6-(1-methyl-4-piperidyloxy)-2-pyridyl]-6-m-toluidino-1,2-dihydro-3H-1,2,5,7-tetraazainden-3-one (40 mg, 33%).

### Example 94. 2-allyl-1-[6-(1-methyl-4-piperidylamino)-2-pyridyl]-6-m-toluidino-1,2-dihydro-3H-1,2,5,7-tetraazainden-3-one (Compound 188)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 144 mg, 56%.

### Example 95. 2-allyl-1-[6-(1-methyl-4-piperidylamino)-2-pyridyl]-6-[4-(2,2,2-trifluoroethoxy)-3-toluidino]-1,2-dihydro-3H-1,2,5,7-tetraazainden-3-one (Compound 181)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 105 mg, 68%.

### Example 96. 2-allyl-1-[6-(1-methyl-4-piperidyloxy)-2-pyridyl]-6-(1-propyl-4-pyrazolylamino)-1,2-dihydro-3H-1,2,5,7-tetraazainden-3-one (Compound 180)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield 75 mg, 70%.

### Example 97. 2-allyl-1-[6-(4-piperidylamino)-2-pyridyl]-6-[4-(2,2,2-trifluoroethoxy)-3-toluidino]-1,2-dihydro-3H-1,2,5,7-tetraazainden-3-one (Compound 178)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 220 mg, 81%.

### Example 98. 1-{6-[N-methyl(1-methyl-4-piperidyl)amino]-2-pyridyl}-2-allyl-6-(1-propyl-4-pyrazolylamino)-1,2-dihydro-3H-1,2,5,7-tetraazainden-3-one (Compound 177)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one. Yield: 8 mg, 10%.

### Example 99. 1-{6-[N—(S)-3-piperidyl-N-methylamino]-2-pyridyl}-2-allyl-6-(p-chlorophenylamino)-1,2-dihydro-3H-1,2,5,7-tetraazainden-3-one (Compound 173)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one starting with tert-butyl(S)-3-amino-1-piperidinecarboxylate. Yield: 7.0 mg TFA salt (31%).

### Example 100. 1-{6-[N—(R)-3-piperidyl-N-methylamino]-2-pyridyl}-2-allyl-6-(p-chlorophenylamino)-1,2-dihydro-3H-1,2,5,7-tetraazainden-3-one (Compound 171)

This compound was made using a similar method as described in the synthesis of 2-allyl-6-(1-methyl-1H-indazol-5-ylamino)-1-[m-(1-methyl-4-piperidylamino)phenyl]-1,2-dihydro-3H-1,2,5,7-tetraazainden-3-one starting with tert-butyl(R)-3-amino-1-piperidinecarboxylate. Yield: 6.6 mg TFA salt (25%).

### Example 101. 1-{6-[(R)-1-methyl-3-piperidylamino]-2-pyridyl}-2-allyl-6-(p-chlorophenylamino)-1,2-dihydro-3H-1,2,5,7-tetraazainden-3-one (Compound 168)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with 1-{6-[(R)-3-piperidylamino]-2-pyridyl}-2-allyl-6-(p-chlorophenylamino)-1,2-dihydro-3H-1,2,5,7-tetraazainden-3-one. Yield: 93 mg, 76%.

### Example 102. 1-{6-[(S)-1-methyl-3-piperidylamino]-2-pyridyl}-2-allyl-6-(p-chlorophenylamino)-1,2-dihydro-3H-1,2,5,7-tetraazainden-3-one (Compound 167)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with 1-{6-[(S)-3-piperidylamino]-2-pyridyl}-2-allyl-6-(p-chlorophenylamino)-1,2-dihydro-3H-1,2,5,7-tetraazainden-3-one. Yield: 91 mg, 34%.

### Example 103. 1-{6-[(R)-3-piperidylamino]-2-pyridyl}-2-allyl-6-(p-chlorophenylamino)-1,2-dihydro-3H-1,2,5,7-tetraazainden-3-one (Compound 166)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with tert-butyl (R)-3-amino-1-piperidinecarboxylate. Yield: 150 mg, 63%.

### Example 104. 1-{6-[(S)-3-piperidylamino]-2-pyridyl}-2-allyl-6-(p-chlorophenylamino)-1,2-dihydro-3H-1,2,5,7-tetraazainden-3-one (Compound 165)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with tert-butyl(S)-3-amino-1-piperidinecarboxylate. Yield: 310 mg, 39%.

### Example 105. 1-(6-{[(S)-1-methyl-3-piperidyl]-N-methylamino}-2-pyridyl)-2-allyl-6-(p-chlorophenylamino)-1,2-dihydro-3H-1,2,5,7-tetraazainden-3-one (Compound 164)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with tert-butyl(S)-3-amino-1-piperidinecarboxylate. Yield: 40 mg TFA salt (43%) as a pale-yellow solid.

### Example 106. (R)-2-allyl-6-((4-chlorophenyl)amino)-1-(6-(methyl(1-methylpiperidin-3-yl)amino)pyridin-2-yl)-1,2-dihydro-3H-pyrazolo[3,4-d]pyrimidin-3-one (Compound 163)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one starting with tert-butyl (R)-3-amino-1-piperidinecarboxylate. Yield: 38 mg TFA salt (35%) as a pale-yellow solid.

### Example 107. 2-allyl-6-(3-fluoro-5-toluidino)-1-[6-(1-methyl-4-piperidylamino)-2-pyridyl]-1,2-dihydro-3H-1,2,5,7-tetraazainden-3-one (Compound 161)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 54 mg, 62%.

### Example 108. 2-allyl-6-(3-fluoro-5-toluidino)-1-[6-(4-piperidylamino)-2-pyridyl]-1,2-dihydro-3H-1,2,5,7-tetraazainden-3-one (Compound 160)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 78 mg, 63%.

### Example 109. 2-allyl-1-[6-(1-methyl-4-piperidylamino)-2-pyridyl]-6-(3-phenyl-5-isothiazolylamino)-1,2-dihydro-3H-1,2,5,7-tetraazainden-3-one (Compound 158)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 1.8 mg, 37%.

### Example 110. 6-(4-fluoro-3-toluidino)-2-methyl-1-[6-(4-piperidyloxy)-2-pyridyl]-1,2-dihydro-3H-1,2,5,7-tetraazainden-3-one (Compound 154)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[2-methyl-6-(methylsulfanyl)-3-oxo-1H,2H,3H-pyrazolo[3,4-d]pyrimidin-1-yl]pyridin-2-yl}oxy)piperidine-1-carboxylate (81 mg) and 4-fluoro-3-methylaniline (1 equiv.) to give the crude free base intermediate (77 mg) of which 20% was purified by prep-HPLC. Yield: 9.3 mg TFA salt (60%).

### Example 111. 6-(p-chlorophenylamino)-2-methyl-1-[6-(4-piperidyloxy)-2-pyridyl]-1,2-dihydro-3H-1,2,5,7-tetraazainden-3-one (Compound 153)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using tert-butyl 4-({6-[2-methyl-6-(methylsulfanyl)-3-oxo-1H,2H,3H-pyrazolo[3,4-d]pyrimidin-1-yl]pyridin-2-yl}oxy)piperidine-1-carboxylate (81 mg) and 4-chloroaniline (1 equiv.) to give the crude free base intermediate (78 mg) of which 20% was purified by prep-HPLC. Yield: 8.7 mg TFA salt (56%).

### Example 112. 6-(4-fluoro-3-toluidino)-2-methyl-1-[6-(1-methyl-4-piperidyloxy)-2-pyridyl]-1,2-dihydro-3H-1,2,5,7-tetraazainden-3-one (Compound 152)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 6-[(4-fluoro-3-methylphenyl)amino]-2-methyl-1-[6-(piperidin-4-yloxy)pyridin-2-yl]-1H,2H,3H-pyrazolo[3,4-d]pyrimidin-3-one (62 mg). Yield: 57 mg TFA salt (71%) as a powder.

### Example 113. 2-allyl-6-(3,4-dichlorophenylamino)-1-[6-(1-methyl-4-piperidylamino)-2-pyridyl]-1,2-dihydro-3H-1,2,5,7-tetraazainden-3-one (Compound 151)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 33 mg, 84%.

### Example 114. 6-(p-chlorophenylamino)-2-methyl-1-[6-(1-methyl-4-piperidyloxy)-2-pyridyl]-1,2-dihydro-3H-1,2,5,7-tetraazainden-3-one (Compound 150)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 45 mg TFA salt (57%) as a powder.

### Example 115. 1-{6-[(S)-1-methyl-3-pyrrolidinyloxy]-2-pyridyl}-2-allyl-6-(p-chlorophenylamino)-1,2-dihydro-3H-1,2,5,7-tetraazainden-3-one (Compound 148)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using tert-butyl (3S)-3-hydroxypyrrolidine-1-carboxylate. Yield: 94 mg TFA salt (82%) as a pale-yellow powder.

### Example 116. 2-allyl-6-(3,4-dichlorophenylamino)-1-[6-(4-piperidylamino)-2-pyridyl]-1,2-dihydro-3H-1,2,5,7-tetraazainden-3-one (Compound 146)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 3,4-dicholoroaniline and tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate. Yield: 46 mg, 24%.

### Example 117. 1-{6-[(S)-3-pyrrolidinyloxy]-2-pyridyl}-2-allyl-6-(p-chlorophenylamino)-1,2-dihydro-3H-1,2,5,7-tetraazainden-3-one (Compound 145)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using tert-butyl (3S)-3-hydroxypyrrolidine-1-carboxylate. Yield: 177 mg TFA salt (62%) as a yellow powder.

### Example 118. 2-allyl-6-(p-chlorophenylamino)-1-[6-(4-piperidyloxy)-2-pyridyl]-1,2-dihydro-3H-1,2,5,7-tetraazainden-3-one (Compound 141)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate (99 mg) and 4-chloroaniline (1 equiv.). After deprotection, the reaction mixture was concentrated to a yellow oil (175 mg, crude), half of which was purified by prep-HPLC then converted to the free base by solid-phase extraction (1 g SCX-2, MeOH, 1.4 M MeOH/NH₃). Yield: 15 mg free base (31%) as a powder.

### Example 119. 2-allyl-6-(p-chlorophenylamino)-1-[6-(1-methyl-4-piperidyloxy)-2-pyridyl]-1,2-dihydro-3H-1,2,5,7-tetraazainden-3-one (Compound 142)

### 2-allyl-1-(6-fluoropyridin-2-yl)-6-(methylthio)-1,2-dihydro-3H-pyrazolo[3,4-d]pyrimidin-3-one

A stirred solution of 2-allyl-6-(methylthio)-1,2-dihydro-3H-pyrazolo[3,4-d]pyrimidin-3-one (1 g, 4.50 mmol), 2-bromo-6-fluoropyridine (0.871 g, 4.95 mmol), trans-N,N-Dimethylethylenediamine (0.397 g, 4.50 mmol) and K₂CO₃ (1.865 g, 13.50 mmol) in DMSO (4 ml), was degassed for 5 min, then was added copper (I) iodide (0.857 g, 4.50 mmol) at 25 °C and the resulting mixture was stirred for 2 h at 100 °C. The progress of the reaction was monitored by TLC. After completion of the reaction, the reaction mixture was filtered on a celite pad and washed with ethyl acetate (250 mL). The filtrate was concentrated under reduced pressure. The resulting crude material was purified by flash chromatography (SiO₂/100-200 mesh; 20-30% Ethyl acetate-hexane) to afford 2-allyl-1-(6-fluoropyridin-2-yl)-6-(methylthio)-1,2-dihydro-3H-pyrazolo[3,4-d]pyrimidin-3-one (700 mg, 2.089 mmol, 46.4% yield) as a pale brown solid.

### 2-allyl-1-(6-fluoropyridin-2-yl)-6-(methylsulfonyl)-1,2-dihydro-3H-pyrazolo[3,4-d]pyrimidin-3-one

To a stirred solution of 2-allyl-1-(6-fluoropyridin-2-yl)-6-(methylthio)-1,2-dihydro-3H-pyrazolo[3,4-d]pyrimidin-3-one (1 g, 3.15 mmol) in dichloromethane (30 mL) was added m-CPBA (1.554 g, 6.30 mmol) at 0 °C and the mixture was stirred for 2 h at 25 °C. The progress of the reaction was monitored by TLC. After completion of the reaction, the reaction mixture was diluted with DCM (300 mL) and washed with sodium bicarbonate solution (2×150 mL). The combined organic layer was dried over Na₂SO₄, filtered and concentrated under reduced pressure to afford the crude 2-allyl-1-(6-fluoropyridin-2-yl)-6-(methylsulfonyl)-1,2-dihydro-3H-pyrazolo[3,4-d]pyrimidin-3-one (1 g, 2.63 mmol, 84% yield) as a pale yellow solid.

### 2-allyl-6-((4-chlorophenyl)amino)-1-(6-fluoropyridin-2-yl)-1,2-dihydro-3H-pyrazolo[3,4-d]pyrimidin-3-one

To a stirred solution of 2-allyl-1-(6-fluoropyridin-2-yl)-6-(methylsulfonyl)-1,2-dihydro-3H-pyrazolo[3,4-d]pyrimidin-3-one (1 g, 2.86 mmol) in acetic acid (10 mL) was added 4-chloroaniline (0.438 g, 3.44 mmol) at room temperature under nitrogen atmosphere. The reaction mixture was stirred at room temperature for 16 h. The progress of the reaction was monitored by TLC. After completion of the reaction, the reaction mixture was concentrated under reduced pressure to afford a residue. The resultant residue was basified with sodium bicarbonate and extracted with ethyl acetate (10×2 mL). The combined organic layer was dried over Na₂SO₄, filtered, and concentrated under reduced pressure to afford crude. The resulting crude material was purified by flash chromatography (SiO₂/100-200 mesh; 7-8% methanol-DCM) to afford 2-allyl-6-((4-chlorophenyl)amino)-1-(6-fluoropyridin-2-yl)-1,2-dihydro-3H-pyrazolo[3,4-d]pyrimidin-3-one (800 mg, 1.774 mmol, 62.0% yield) as a pale brown solid.

### 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one (Compound 142)

NaH (60% in Oil) (121 mg, 2.77 mmol) was added to a stirred solution of 1-methylpiperidin-4-ol (218 mg, 1.890 mmol) in tetrahydrofuran (10 ml). The resulting mixture was stirred for 1 h at 60 °C. Then 2-allyl-6-((4-chlorophenyl)amino)-1-(6-fluoropyridin-2-yl)-1,2-dihydro-3H-pyrazolo[3,4-d]pyrimidin-3-one (6, 500 mg, 1.260 mmol) was added and the mixture stirred at 60 °C for 16 h. The progress of the reaction was monitored by TLC. After completion of the reaction, the reaction mixture was diluted with ice water (150 mL) and extracted with 10% methanol-DCM (2×100 mL). The combined organic layer was dried over Na₂SO₄, filtered, and concentrated under reduced pressure. The resulting crude material was purified by flash chromatography (SiO₂/230-400 mesh; 20-25% methanol-DCM) to afford 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one (175 mg, 0.352 mmol, 27.9% yield) as an off white solid.

### Example 120. 2-allyl-6-(4-fluoro-3-toluidino)-1-[6-(1-methyl-4-piperidylamino)-2-pyridyl]-1,2-dihydro-3H-1,2,5,7-tetraazainden-3-one (Compound 140)

This compound was made using a similar method as described in the synthesis of This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 129 mg, 75%.

### Example 121. 2-allyl-1-[6-(1-methyl-4-piperidylamino)-2-pyridyl]-6-[p-(2,2,2-trifluoroethoxy)phenylamino]-1,2-dihydro-3H-1,2,5,7-tetraazainden-3-one (Compound 139)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 80 mg, 66.6%.

### Example 122. 2-allyl-6-(p-chlorophenylamino)-1-[6-(1-methyl-4-piperidylamino)-2-pyridyl]-1,2-dihydro-3H-1,2,5,7-tetraazainden-3-one (Compound 138)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 7.4 mg, 63%.

### Example 123. 2-allyl-1-[6-(1-methyl-4-piperidyloxy)-2-pyridyl]-6-(1-methyl-4-pyrazolylamino)-1,2-dihydro-3H-1,2,5,7-tetraazainden-3-one (Compound 137)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield: 20 mg, 17%.

### Example 124. 2-allyl-6-(4-chloro-3-toluidino)-1-[6-(4-piperidylamino)-2-pyridyl]-1,2-dihydro-3H-1,2,5,7-tetraazainden-3-one (Compound 133)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 4-chloro-3-methylaniline and tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate. Yield: 24 mg, 38%.

### Example 125. 2-ethyl-1-[6-(1-methyl-4-piperidyloxy)-2-pyridyl]-6-(1-methyl-4-pyrazolylamino)-1,2-dihydro-3H-1,2,5,7-tetraazainden-3-one (Compound 132)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-methyl-1H-pyrazol-4-amine and 2-ethyl-1-{6-[(1-methylpiperidin-4-yl)oxy]pyridin-2-yl}-6-(methylsulfanyl)-1H,2H,3H-pyrazolo[3,4-d]pyrimidin-3-one. Yield: 35 mg, 56%.

### Example 126. 2-allyl-1-[6-(4-piperidylamino)-2-pyridyl]-6-[p-(trifluoromethyl)phenylamino]-1,2-dihydro-3H-1,2,5,7-tetraazainden-3-one (Compound 131)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 18 mg, 29%.

### Example 127. 2-allyl-1-[6-(4-piperidylamino)-2-pyridyl]-6-[p-(2,2,2-trifluoroethoxy)phenylamino]-1,2-dihydro-3H-1,2,5,7-tetraazainden-3-one (Compound 130)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 6.9 mg.

### Example 128. 2-allyl-6-(4-fluoro-3-toluidino)-1-[6-(4-piperidylamino)-2-pyridyl]-1,2-dihydro-3H-1,2,5,7-tetraazainden-3-one (Compound 128)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 4-fluoro-3-methylaniline and tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate. Yield: 153 mg, 61%.

### Example 129. 1-{6-[N-methyl(1-methyl-4-piperidyl)amino]-2-pyridyl}-2-ethyl-6-(1-methyl-1H-indazol-5-ylamino)-1,2-dihydro-3H-1,2,5,7-tetraazainden-3-one (Compound 287)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 35 mg, 60%.

### Example 130. 2-allyl-6-(4-methoxy-3-toluidino)-1-[6-(4-piperidylamino)-2-pyridyl]-1,2-dihydro-3H-1,2,5,7-tetraazainden-3-one (Compound 127)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 4-methoxy-3-methylaniline and tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate. Yield: 43 mg, 71%.

### Example 131. 2-allyl-1-[6-(4-piperidylamino)-2-pyridyl]-6-m-toluidino-1,2-dihydro-3H-1,2,5,7-tetraazainden-3-one (Compound 126)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 3-methylaniline and tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate. Yield: 39 mg, 67%.

### Example 132. 2-allyl-6-(p-chlorophenylamino)-1-[6-(4-piperidylamino)-2-pyridyl]-1,2-dihydro-3H-1,2,5,7-tetraazainden-3-one (Compound 124)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg) and 4-chloroaniline (1 equiv.). Yield: 29 mg TFA salt (49%) as a yellow powder.

### Example 133. 1-[6-(N-methyl-N-4-piperidylamino)-2-pyridyl]-2-ethyl-6-(1-methyl-1H-indazol-5-ylamino)-1,2-dihydro-3H-1,2,5,7-tetraazainden-3-one (Compound 286)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-methyl-1H-indazol-5-amine and tert-butyl 4-({6-[2-ethyl-6-(methylsulfanyl)-3-oxo-1H,2H,3H-pyrazolo[3,4-d]pyrimidin-1-yl]pyridin-2-yl}(methyl)amino)piperidine-1-carboxylate. Yield: 70 mg, 54%.

### Example 134. 2-methyl-6-(1-methyl-1H-indazol-5-ylamino)-1-[6-(1-methyl-4-piperidyloxy)-2-pyridyl]-1,2-dihydro-3H-1,2,5,7-tetraazainden-3-one (Compound 121)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 2-methyl-1-{6-[(1-methylpiperidin-4-yl)oxy]pyridin-2-yl}-6-(methylsulfanyl)-1H,2H,3H-pyrazolo[3,4-d]pyrimidin-3-one (69 mg) and 1-methyl-1H-indazol-5-amine (1 equiv.). Yield: 49 mg TFA salt (46%) as a yellow powder.

### Example 135. 1-{6-[N-methyl(1-methyl-4-piperidyl)amino]-2-pyridyl}-2-allyl-6-(1-methyl-1H-indazol-5-ylamino)-1,2-dihydro-3H-1,2,5,7-tetraazainden-3-one (Compound 118)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 9.8 mg, 68%.

### Example 136. 6-anilino-2-ethyl-1-[6-(1-methyl-4-piperidyloxy)-2-pyridyl]-1,2-dihydro-3H-1,2,5,7-tetraazainden-3-one (Compound 117)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 2-ethyl-1-{6-[(1-methylpiperidin-4-yl)oxy]pyridin-2-yl}-6-(methylsulfanyl)-1H,2H,3H-pyrazolo[3,4-d]pyrimidin-3-one (102 mg) and aniline (1 equiv.). Yield: 69 mg TFA salt (48%) as a yellow powder.

### Example 137. 1-[6-(N-methyl-N-4-piperidylamino)-2-pyridyl]-2-allyl-6-(1-methyl-1H-indazol-5-ylamino)-1,2-dihydro-3H-1,2,5,7-tetraazainden-3-one (Compound 116)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-methyl-1H-indazol-5-amine and tert-butyl 4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate. Yield: 22 mg, 40%.

### Example 138. 2-ethyl-6-(1-methyl-1H-indazol-5-ylamino)-1-[6-(1-methyl-4-piperidyloxy)-2-pyridyl]-1,2-dihydro-3H-1,2,5,7-tetraazainden-3-one (Compound 114)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 2-ethyl-1-{6-[(1-methylpiperidin-4-yl)oxy]pyridin-2-yl}-6-(methylsulfanyl)-1H,2H,3H-pyrazolo[3,4-d]pyrimidin-3-one (103 mg) and 1-methyl-1H-indazol-5-amine (2.5 equiv.). Yield: 64 mg TFA salt (41%) as a yellow powder.

### Example 139. 2-allyl-6-(2-methyl-4-pyridylamino)-1-[6-(4-piperidyloxy)-2-pyridyl]-1,2-dihydro-3H-1,2,5,7-tetraazainden-3-one (Compound 113)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield 2 mg.

### Example 140. 1-{6-[(S)-1-methyl-3-pyrrolidinylamino]-2-pyridyl}-2-allyl-6-(1-methyl-1H-indazol-5-ylamino)-1,2-dihydro-3H-1,2,5,7-tetraazainden-3-one (Compound 108)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 6.5 mg TFA salt (24%) as a yellow powder.

### Example 141. Synthesis of 1-{6-[(S)-3-pyrrolidinylamino]-2-pyridyl}-2-allyl-6-(1-methyl-1H-indazol-5-ylamino)-1,2-dihydro-3H-1,2,5,7-tetraazainden-3-one (Compound 107)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield: 73 mg TFA salt (66%) as a yellow powder.

### Example 142. Synthesis of 2-allyl-6-(1-methyl-1H-indazol-5-ylamino)-1-[6-(4-piperidyloxy)-2-pyridyl]-1,2-dihydro-3H-1,2,5,7-tetraazainden-3-one (Compound 106)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate (184 mg) and 1-methyl-1H-indazol-5-amine (1 equiv.). Yield: 140 mg TFA salt (62%) as a yellow powder.

### Example 143. Synthesis of 2-allyl-6-(1-methyl-1H-indazol-5-ylamino)-1-[6-(1-methyl-4-piperidyloxy)-2-pyridyl]-1,2-dihydro-3H-1,2,5,7-tetraazainden-3-one (Compound 109)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 6-[(1-methyl-1H-indazol-5-yl)amino]-1-[6-(piperidin-4-yloxy)pyridin-2-yl]-2-(prop-2-en-1-yl)-1H,2H,3H-pyrazolo[3,4-d]pyrimidin-3-one; trifluoroacetic acid salt (15 mg). Yield: 9 mg TFA salt (60%) as a pale-yellow powder.

### Example 144. Synthesis of 1-{6-[(S)-1-methyl-3-pyrrolidinyloxy]-2-pyridyl}-2-allyl-6-(1-methyl-1H-indazol-5-ylamino)-1,2-dihydro-3H-1,2,5,7-tetraazainden-3-one (Compound 105)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using tert-butyl (3S)-3-hydroxypyrrolidine-1-carboxylate. Yield: 10.4 mg, 62%.

### Example 145. Synthesis of 1-{6-[(S)-3-piperidyloxy]-2-pyridyl}-2-allyl-6-(1-methyl-1H-indazol-5-ylamino)-1,2-dihydro-3H-1,2,5,7-tetraazainden-3-one (Compound 104)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 1-methyl-1H-indazol-5-amine (1 equiv.) and tert-butyl (3S)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate (135 mg) prepared from tert-butyl (3S)-3-hydroxypiperidine-1-carboxylate. Yield: 92 mg TFA salt (56%) as a yellow powder.

### Example 146. Synthesis of 1-{6-[(R)-3-pyrrolidinyloxy]-2-pyridyl}-2-allyl-6-(1-methyl-1H-indazol-5-ylamino)-1,2-dihydro-3H-1,2,5,7-tetraazainden-3-one (Compound 101)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 1-methyl-1H-indazol-5-amine and tert-butyl (3R)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy) pyrrolidine-1-carboxylate made from tert-butyl (3R)-3-hydroxypyrrolidine-1-carboxylate. Yield: 8.1 mg, 21%.

### Example 147. Synthesis of 1-{6-[(S)-3-pyrrolidinyloxy]-2-pyridyl}-2-allyl-6-(1-methyl-1H-indazol-5-ylamino)-1,2-dihydro-3H-1,2,5,7-tetraazainden-3-one (Compound 100)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 1-methyl-1H-indazol-5-amine and tert-butyl (3S)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy) pyrrolidine-1-carboxylate made from tert-butyl (3S)-3-hydroxypyrrolidine-1-carboxylate. Yield: 55.1 mg, 60%.

### Example 148. Synthesis of 2-allyl-6-(1-methyl-1H-1,3-benzimidazol-5-ylamino)-1-[6-(1-methyl-4-piperidyloxy)-2-pyridyl]-1,2-dihydro-3H-1,2,5,7-tetraazainden-3-one (Compound 211)

mCPBA (<77% pure) (41.5 mg, assumed 0.241 mmol) in DCM (0.5 mL) was added to a stirred solution of tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (100 mg, 0.201 mmol) in DCM (2.5 mL) at room temperature under nitrogen. The reaction controlled by LCMS. After 15 min, DCM was removed in vacuo, and the crude solubilized in MeCN (3 ml). Then 1-methyl-1H-1,3-benzimidazol-5-ylamine (29.5 mg, 0.201 mmol) and methane sulfonic acid (26 μL, 0.401 mmol) were added. The reaction mixture stirred at 60 °C in a closed vial. After 18 h, reaction mixture was allowed to cool to RT, and mCPBA quenched with 1M NaOH (5 mL) which was added dropwise. The aqueous phase was extracted with EtOAc (3×20 mL) then brine (20 mL). The combined organic layers were dried (MgSO₄) and concentrated under reduced pressure to give the product as a yellow powder. The crude material was dissolved in DCM (5 ml), loaded onto a 10 g silica column and purified by flash chromatography (0-100%, EtOAc: PE) to give the Boc protected compound [85 mg, 94%] as a pale-yellow solid. This material (85 mg, 0.131 mmol) in DCM (5 mL) was treated with TFA (2.5 mL) for 2 h. The solvent was removed in vacuo and the crude was dissolved in EtOAc, washed with sat aq NaHCO₃, brine, dried (MgSO₄), and concentrated. The resulting material was purified by with reversed phase chromatography (Gemini NX—C18,21*150 mm, water (0.1% TFA)/acetonitrile, gradient over 12 minutes, 25 ml/min). The pure fractions were pooled and concentrated to give 2-allyl-6-(1-methyl-1H-1,3-benzimidazol-5-ylamino)-1-[6-(4-piperidyloxy)-2-pyridyl]-1,2-dihydro-3H-1,2,5,7-tetraazainden-3-one as a trifluoroacetic acid salt as a white solid [85 mg].

### Example 149. Synthesis of 2-allyl-6-((1-methyl-1H-indol-5-yl)amino)-1-(6-((1-methylpiperidin-4-yl)oxy)pyridin-2-yl)-1,2-dihydro-3H-pyrazolo[3,4-d]pyrimidin-3-one (Compound 186)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 1-{6-[(1-methylpiperidin-4-yl)oxy]pyridin-2-yl}-6-(methyl sulfanyl)-2-(prop-2-en-1-yl)-1H,2H,3H-pyrazolo[3,4-d]pyrimidin-3-one (100 mg) and 1-methyl-1H-indol-5-amine (1.5 equiv.). Yield: 77 mg TFA salt (51%) as a powder.

### Example 150. Synthesis of 1-{6-[(R)-3-pyrrolidinylamino]-2-pyridyl}-2-allyl-6-(1-methyl-1H-indazol-5-ylamino)-1,2-dihydro-3H-1,2,5,7-tetraazainden-3-one (Compound 102)

### tert-butyl (3R)-3-[(6-bromopyridin-2-yl)amino]pyrrolidine-1-carboxylate

2-Bromo-6-fluoropyridine (0.37 g, 2.1 mmol), DIPEA (6.3 mmol, 1.1 ml) and tert-butyl (3R)-3-aminopyrrolidine-1-carboxylate hydrochloride (0.52 g, 2.1 mmol) were mixed in 10 ml of DMSO. The reaction mixture was stirred at 100 °C for two days and partitioned between ethyl acetate and water. The organic phase was washed with water, sat. NaHCO₃ and brine, dried over MgSO₄, filtered and concentrated. The residue was slurried with a mixture of heptane and ethyl acetate and collected by filtration giving 0.42 g (58%) of tert-butyl (3R)-3-[(6-bromopyridin-2-yl)amino]pyrrolidine-1-carboxylate.

### Step 2

6-(Methylsulfanyl)-2-(prop-2-en-1-yl)-1H,2H,3H-pyrazolo[3,4-d]pyrimidin-3-one (56 mg, 0.25 mmol), copper (I) iodide (52 mg, 0.27 mmol), potassium carbonate (104 mg, 0.75 mmol), 1,2-dimethylethylenediamine (44 mg, 0.50 mmol) and tert-butyl (3R)-3-[(6-bromopyridin-2-yl)amino]pyrrolidine-1-carboxylate (86 mg, 0.25 mmol) were mixed in 20 ml of dioxane under nitrogen. The reaction mixture was stirred at 90 °C overnight, diluted with ethyl acetate, filtered through celite and concentrated. The residue was purified with flash chromatography (silica, 10-40% ethyl acetate in petroleum ether).

### 1-{6-[(R)-3-pyrrolidinylamino]-2-pyridyl}-2-allyl-6-(1-methyl-1H-indazol-5-ylamino)-1,2-dihydro-3H-1,2,5,7-tetraazainden-3-one

The compound from Step 2 and MCPBA (77%, 31 mg, 0.14 mmol) were mixed in 5 ml of DCM. After two hours 1-methyl-1H-indazol-5-amine (37 mg, 0.25 mmol) was added. The reaction mixture was stirred at room temperature overnight. TFA (2 ml) was added and after 3 hours the solvent removed under reduced pressure. The residue was dissolved in methanol/water and purified with reversed phase chromatography (Gemini NX—C18, 21*150 mm, water (0.1% TFA)/acetonitrile, gradient over 12 minutes, 25 ml/min). The pure fractions were pooled and concentrated giving 13 mg (7% over two steps) of the title compound.

### Example 151. Synthesis of 6-[(1-Methyl-1H-indazol-5-yl)amino]-1-{6-[(3R)-piperidin-3-yloxy]pyridin-2-yl}-2-(prop-2-en-1-yl)-1H,2H,3H-pyrazolo[3,4-d]pyrimidin-3-one; bis(trifluoroacetic acid) (Compound 103)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one using 1-methyl-1H-indazol-5-amine and tert-Butyl (3R)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate. Yield: 130 mg, 56%.

### Example 152. Synthesis of 6-[(1-methyl-1H-indazol-5-yl)amino]-1-[6-(piperidin-4-yloxy)pyridin-2-yl]-2-propyl-1H,2H,3H-pyrazolo[3,4-d]pyrimidin-3-one (Compound 110)

To a solution of 6-[(1-methyl-1H-indazol-5-yl)amino]-1-[6-(piperidin-4-yloxy)pyridin-2-yl]-2-(prop-2-en-1-yl)-1H,2H,3H-pyrazolo[3,4-d]pyrimidin-3-one; trifluoroacetic acid (60 mg, 0.098 mmol) in ethanol (5 mL) was added 10% palladium on charcoal (4.4 mg) and the mixture was stirred under hydrogen atmosphere (1 atm) at room temperature overnight. The solution was filtered through celite to remove catalyst and the celite pad was washed with methanol. The filtrate was concentrated under reduced pressure to give the crude product as a colorless oil. The crude product was purified by reversed phase chromatography and the pure fractions were pooled and lyophilized to give the title compound. Yield: 20 mg TFA salt (33%).

### Example 153. Synthesis of 6-[(1-methyl-1H-indazol-5-yl)amino]-1-{6-[(1-methylpiperidin-4-yl)oxy]pyridin-2-yl}-2-propyl-1H,2H,3H-pyrazolo[3,4-d]pyrimidin-3-one (Compound 111)

To a solution of 6-[(1-methyl-1H-indazol-5-yl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one; trifluoroacetic acid (41 mg, 0.066 mmol) in ethanol (4 mL) was added 10% palladium on charcoal (2.95 mg) and the mixture was stirred under hydrogen atmosphere (1 atm) at room temperature overnight. The solution was filtered through celite to remove catalyst and the celite pad was washed with methanol. The filtrate was concentrated under reduced pressure to give the crude product as a colorless oil, which was dissolved in acetonitrile and water, and lyophilized to give the title compound. Yield: 33 mg TFA salt (80%).

### Example 154. Synthesis of 1-{6-[(1-methylpiperidin-4-yl)oxy]pyridin-2-yl}-6-[(2-methylpyridin-4-yl)amino]-2-(prop-2-en-1-yl)-1H,2H,3H-pyrazolo[3,4-d]pyrimidin-3-one (Compound 112)

This compound was made using a similar method as described in the synthesis of 6-[(1-methyl-1H-indazol-5-yl)amino]-1-{6-[(1-methylpiperidin-4-yl)oxy]pyridin-2-yl}-2-propyl-1H,2H,3H-pyrazolo[3,4-d]pyrimidin-3-one using 6-[(2-methylpyridin-4-yl)amino]-1-[6-(piperidin-4-yloxy)pyridin-2-yl]-2-(prop-2-en-1-yl)-1H,2H,3H-pyrazolo[3,4-d]pyrimidin-3-one. Yield: 31 mg TFA salt (28%) as a powder.

### Example 155. Synthesis of 1-{6-[(R)-1-methyl-3-pyrrolidinyloxy]-2-pyridyl}-2-allyl-6-(1-methyl-1H-indazol-5-ylamino)-1,2-dihydro-3H-1,2,5,7-tetraazainden-3-one (Compound 115)

### 2-allyl-1-(6-fluoropyridin-2-yl)-6-(methylsulfinyl)-1,2-dihydro-3H-pyrazolo[3,4-d]pyrimidin-3-one

To a stirred solution of 2-allyl-1-(6-fluoropyridin-2-yl)-6-(methylthio)-1,2-dihydro-3H-pyrazolo[3,4-d]pyrimidin-3-one (1 g, 3.15 mmol) in DCM (10 ml) was added m-CPBA (1.088 g, 6.30 mmol), the resulting reaction mixture was stirred at RT for 3 h. The progress of the reaction was monitored by TLC. After completion of the reaction, the reaction mixture was quenched with sodium bicarbonate and extracted with DCM to get crude (1 g, 1.920 mmol, 60.9% yield) which was taken as such for the next step.

### 2-allyl-1-(6-fluoropyridin-2-yl)-6-((1-methyl-1H-indazol-5-yl)amino)-1,2-dihydro-3H-pyrazolo[3,4-d]pyrimidin-3-one

To a stirred solution of 2-allyl-1-(6-fluoropyridin-2-yl)-6-(methylsulfonyl)-1,2-dihydro-3H-pyrazolo[3,4-d]pyrimidin-3-one (1 g, 2.86 mmol) in acetic acid (10 ml) was added 1-methyl-1H-indazol-5-amine (3, 0.421 g, 2.86 mmol). The resulting reaction mixture was stirred at RT for 16 h. The progress of the reaction was monitored by TLC. After completion of the reaction, the reaction mixture was concentrated and quenched with ice-cold water, appeared solid was filtered and washed with MTBE (20 mL) to get the pure compound 2-allyl-1-(6-fluoropyridin-2-yl)-6-((1-methyl-1H-indazol-5-yl)amino)-1,2-dihydro-3H-pyrazolo[3,4-d]pyrimidin-3-one (800 mg, 1.767 mmol, 61.7% yield as brown solid.

### (R)-2-allyl-6-((1-methyl-1H-indazol-5-yl)amino)-1-(6-((1-methylpyrrolidin-3-yl)oxy)pyridin-2-yl)-1,2-dihydro-3H-pyrazolo[3,4-d]pyrimidin-3-one

To a stirred solution of (R)-1-methylpyrrolidin-3-ol (5, 300 mg, 2.97 mmol) in THF (5 ml) was added NaH (60% in oil) (214 mg, 8.90 mmol) at 60 °C and stirred for 1 h. Then 2-allyl-1-(6-fluoropyridin-2-yl)-6-((1-methyl-1H-indazol-5-yl)amino)-1,2-dihydro-3H-pyrazolo[3,4-d]pyrimidin-3-one (4, 300 mg, 0.720 mmol) was added to the above reaction and allowed to stirred at RT for 16 h. The progress of the reaction was monitored by TLC After completion of the reaction. Reaction was quenched with ice-cold water and extracted with DCM to get crude and concentrated under reduced pressure then submitted to prep HPLC (X-SELECT C18 150M, 0.1% FA IN H₂O), collected fractions after lyophilization to get pure (R)-2-allyl-6-((1-methyl-1H-indazol-5-yl)amino)-1-(6-((1-methylpyrrolidin-3-yl)oxy)pyridin-2-yl)-1,2-dihydro-3H-pyrazolo[3,4-d]pyrimidin-3-one (90 mg, 0.179 mmol, 6.04% yield).

### Example 156. Synthesis of 1-(6-{[(3R)-1-ethylpyrrolidin-3-yl]amino}pyridin-2-yl)-6-[(1-methyl-1H-indazol-5-yl)amino]-2-(prop-2-en-1-yl)-1H,2H,3H-pyrazolo[3,4-d]pyrimidin-3-one (Compound 119)

This compound was made using a similar method as described in the synthesis of 2-allyl-6-(p-bromophenylamino)-1-[6-(1-methyl-4-piperidyloxy)-2-pyridyl]-1,2-dihydro-3H-1,2,5,7-tetraazainden-3-one using 6-[(1-methyl-1H-indazol-5-yl)amino]-2-(prop-2-en-1-yl)-1-(6-{[(3R)-pyrrolidin-3-yl]amino}pyridin-2-yl)-1H,2H,3H-pyrazolo[3,4-d]pyrimidin-3-one; trifluoroacetic acid (45 mg) and acetaldehyde (excess). Yield: 15 mg TFA salt (32%) as a powder.

### Example 157. Synthesis of 6-[(1-methyl-1H-indazol-5-yl)amino]-2-(prop-2-en-1-yl)-1-(6-{[(3R)-1-(propan-2-yl) pyrrolidin-3-yl]amino}pyridin-2-yl)-1H,2H,3H-pyrazolo[3,4-d]pyrimidin-3-one (Compound 120)

This compound was made using a similar method as described in the synthesis of 2-allyl-6-(p-bromophenylamino)-1-[6-(1-methyl-4-piperidyloxy)-2-pyridyl]-1,2-dihydro-3H-1,2,5,7-tetraazainden-3-one using 6-[(1-methyl-1H-indazol-5-yl)amino]-2-(prop-2-en-1-yl)-1-(6-{[(3R)-pyrrolidin-3-yl]amino}pyridin-2-yl)-1H,2H,3H-pyrazolo[3,4-d]pyrimidin-3-one; trifluoroacetic acid (45 mg) and acetone (excess). Yield: 30 mg TFA salt (63%) as a powder.

### Example 158. Synthesis of 6-[(1-methyl-1H-indazol-5-yl)amino]-1-{6-[(piperidin-4-yl)amino]pyridin-2-yl}-2-(prop-2-en-1-yl)-1H,2H,3H-pyrazolo[3,4-d]pyrimidin-3-one (Compound 122)

This compound was made using a similar method as described in the synthesis of 2-allyl-6-((4-chlorophenyl)amino)-1-(6-(piperidin-4-ylamino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (182 mg) and 1-methyl-1H-indazol-5-amine (1 equiv.). Yield: 107 mg TFA salt (48%) as a yellow powder.

### Example 159. Synthesis of 6-(phenylamino)-1-{6-[(piperidin-4-yl)amino]pyridin-2-yl}-2-(prop-2-en-1-yl)-1H,2H,3H-pyrazolo[3,4-d]pyrimidin-3-one (Compound 123)

This compound was made using a similar method as described in the synthesis of 2-allyl-6-((4-chlorophenyl)amino)-1-(6-(piperidin-4-ylamino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg) and aniline (3 equiv.). Yield: 31 mg TFA salt (55%) as a yellow powder.

### Example 160. Synthesis of 6-[(4-fluorophenyl)amino]-1-{6-[(piperidin-4-yl)amino]pyridin-2-yl}-2-(prop-2-en-1-yl)-1H,2H,3H-pyrazolo[3,4-d]pyrimidin-3-one (Compound 125)

This compound was made using a similar method as described in the synthesis of 2-allyl-6-((4-chlorophenyl)amino)-1-(6-(piperidin-4-ylamino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg) and 4-fluoroaniline (2 equiv.). Yield: 37 mg TFA salt (64%) as a powder.

### Example 161. Synthesis of 6-[(1-methyl-1H-indazol-5-yl)amino]-1-{6-[(1-methylpiperidin-4-yl)amino]pyridin-2-yl}-2-(prop-2-en-1-yl)-1H,2H,3H-pyrazolo[3,4-d]pyrimidin-3-one (Compound 129)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 6-[(1-methyl-1H-indazol-5-yl)amino]-1-{6-[(piperidin-4-yl)amino]pyridin-2-yl}-2-(prop-2-en-1-yl)-1H,2H,3H-pyrazolo[3,4-d]pyrimidin-3-one; trifluoroacetic acid (38 mg). Yield: 20 mg (52%) as a powder.

### Example 162. Synthesis of 2-methyl-4-[(3-oxo-1-{6-[(piperidin-4-yl)amino]pyridin-2-yl}-2-(prop-2-en-1-yl)-1H,2H,3H-pyrazolo[3,4-d]pyrimidin-6-yl)amino]benzonitrile (Compound 134)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using tert-butyl 4-[(6-{6-[(4-cyano-3-methylphenyl)amino]-3-oxo-2-(prop-2-en-1-yl)-1H,2H,3H-pyrazolo[3,4-d]pyrimidin-1-yl}pyridin-2-yl)oxy]piperidine-1-carboxylate followed by deprotection of the boc-group. Yield: 15.3 mg TFA salt (39%) as a white powder.

### Example 163. Synthesis of 1-{6-[(piperidin-4-yl)amino]pyridin-2-yl}-2-(prop-2-en-1-yl)-6-{[4-(trifluoromethoxy)phenyl]amino}-1H,2H,3H-pyrazolo[3,4-d]pyrimidin-3-one (Compound 135)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg) and 4-(trifluoromethoxy)aniline (2 equiv.). Yield: 27 mg (51%) as a powder.

### Example 164. Synthesis of 6-{[3-methyl-4-(trifluoromethoxy)phenyl]amino}-1-{6-[(piperidin-4-yl)amino]pyridin-2-yl}-2-(prop-2-en-1-yl)-1H,2H,3H-pyrazolo[3,4-d]pyrimidin-3-one (Compound 136)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg) and 3-methyl-4-(trifluoromethoxy)aniline (2 equiv.). Yield: 29 mg TFA salt (44%) as a powder.

### Example 165. Synthesis of 6-[(4-bromophenyl)amino]-1-{6-[(piperidin-4-yl)amino]pyridin-2-yl}-2-(prop-2-en-1-yl)-1H,2H,3H-pyrazolo[3,4-d]pyrimidin-3-one (Compound 143)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (60 mg) and 4-bromoaniline (2 equiv.). Yield: 27.5 mg (44%) as a white powder.

### Example 166. Synthesis of 6-[(3-bromo-5-chlorophenyl)amino]-1-{6-[(piperidin-4-yl)amino]pyridin-2-yl}-2-(prop-2-en-1-yl)-1H,2H,3H-pyrazolo[3,4-d]pyrimidin-3-one (Compound 144)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (50 mg), 3-bromo-5-chloroaniline (2 equiv.) and methanesulfonic acid (1 equiv.). Yield: 25 mg TFA salt (37%) as a powder.

### Example 167. Synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one (Compound 149)

### tert-butyl 4-[(6-bromopyridin-2-yl)(propyl)amino]piperidine-1-carboxylate

Sodium hydride 60% in mineral oil (113 mg, 2.82 mmol, 1.3 equiv.) was added to as stirred solution of tert-butyl 4-[(6-bromopyridin-2-yl)amino]piperidine-1-carboxylate (773 mg, 2.17 mmol, 1 equiv.) and propyl methanesulfonate (360 mg, 2.6 mmol, 1.2 equiv.) in DMF (8 mL) at 0 °C. Upon complete addition, the mixture was brought to room temperature and stirred for 40 min. The reaction mixture was diluted with water (50 mL) and extracted with ethyl acetate (3×120 mL). The organic layer was washed with water (4×100 mL), brine (100 mL) and then dried over magnesium sulfate. The organic layer was concentrated to residues under reduced pressure. The residues were purified by flash chromatography on silica, eluting with a gradient of 5-70% ethyl acetate in petroleum ether to give the title compound (470 mg, 54%) as a colorless oil.

### tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}(propyl)amino)piperidine-1-carboxylate

This intermediate was made using a similar method as described in the synthesis of tert-butyl 4-((6-(2-allyl-6-(methylthio)-3-oxo-2,3-dihydro-1H-pyrazolo[3,4-d]pyrimidin-1-yl)pyridin-2-yl)amino)piperidine-1-carboxylate using tert-butyl 4-[(6-bromopyridin-2-yl)(propyl)amino]piperidine-1-carboxylate (475 mg). Yield: 519 mg (81%).

### 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one

The title compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 6-[(4-chlorophenyl)amino]-1-{6-[(piperidin-4-yl)(propyl)amino]pyridin-2-yl}-2-(prop-2-en-1-yl)-1H,2H,3H-pyrazolo[3,4-d]pyrimidin-3-one; trifluoroacetic acid (85 mg). Yield: 65 mg TFA salt (75%) as a powder.

### Example 168. Synthesis of 6-{[3-methyl-5-(trifluoromethoxy)phenyl]amino}-1-{6-[(piperidin-4-yl)amino]pyridin-2-yl}-2-(prop-2-en-1-yl)-1H,2H,3H-pyrazolo[3,4-d]pyrimidin-3-one (Compound 155)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (100 mg) and 3-methyl-5-(trifluoromethoxy)aniline (1.5 equiv.). Yield: 60 mg TFA salt (46%) as a powder.

### Example 169. Synthesis of 6-[(4-chlorophenyl)amino]-1-{6-[methyl(piperidin-4-yl)amino]pyridin-2-yl}-2-(prop-2-en-1-yl)-1H,2H,3H-pyrazolo[3,4-d]pyrimidin-3-one (Compound 156)

This compound was made using a similar method as described in the synthesis of: 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using tert-butyl 4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate (200 mg) and 4-chloroaniline (1 equiv.). Yield: 112 mg (58%) as a white powder.

### Example 170. Synthesis of 6-[(4-chlorophenyl)amino]-1-{6-[methyl(1-methylpiperidin-4-yl)amino]pyridin-2-yl}-2-(prop-2-en-1-yl)-1H,2H,3H-pyrazolo[3,4-d]pyrimidin-3-one (Compound 157)

This compound was made using a similar method as described in the synthesis of: 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using 6-[(4-chlorophenyl)amino]-1-{6-[methyl(piperidin-4-yl)amino]pyridin-2-yl}-2-(prop-2-en-1-yl)-1H,2H,3H-pyrazolo[3,4-d]pyrimidin-3-one (65 mg). Yield: 68 mg TFA salt (83%) as a pale-yellow powder.

### Example 171. Synthesis of 6-[(4-chlorophenyl)amino]-1-{6-[(3R)-piperidin-3-yloxy]pyridin-2-yl}-2-(prop-2-en-1-yl)-1H,2H,3H-pyrazolo[3,4-d]pyrimidin-3-one (Compound 159)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate (166 mg) and 4-chloroaniline (1 equiv.). Yield: 150 mg TFA salt (76%) as a yellow powder.

### Example 172. Synthesis of 6-[(4-chlorophenyl)amino]-1-(6-{[(3S)-1-methylpiperidin-3-yl]oxy}pyridin-2-yl)-2-(prop-2-en-1-yl)-1H,2H,3H-pyrazolo[3,4-d]pyrimidin-3-one (Compound 162)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 6-[(4-chlorophenyl)amino]-1-{6-[(3R)-piperidin-3-yloxy]pyridin-2-yl}-2-(prop-2-en-1-yl)-1H,2H,3H-pyrazolo[3,4-d]pyrimidin-3-one; trifluoroacetic acid (kdh-0088) (150 mg). Yield: 82 mg free base (53%) as a powder.

### Example 173. Synthesis of 1-{6-[methyl(piperidin-4-yl)amino]pyridin-2-yl}-2-(prop-2-en-1-yl)-6-{[1-(propan-2-yl)-1H-pyrazol-4-yl]amino}-1H,2H,3H-pyrazolo[3,4-d]pyrimidin-3-one (Compound 169)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using tert-butyl 4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate (200 mg) and 1-isopropyl-4-pyrazolamine (1.5 equiv.). Yield: 139 mg TFA salt (59%) as a powder.

### Example 174. Synthesis of 1-{6-[methyl(piperidin-4-yl)amino]pyridin-2-yl}-6-{[1-(2-methylpropyl)-1H-pyrazol-4-yl]amino}-2-(prop-2-en-1-yl)-1H,2H,3H-pyrazolo[3,4-d]pyrimidin-3-one (Compound 170)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using tert-butyl 4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate (200 mg) and 1-isobutyl-4-pyrazolamine (1.5 equiv.). Yield: 153 mg TFA salt (63%) as a powder.

### Example 175. Synthesis of 1-{6-[methyl(1-methylpiperidin-4-yl)amino]pyridin-2-yl}-2-(prop-2-en-1-yl)-6-{[1-(propan-2-yl)-1H-pyrazol-4-yl]amino}-1H,2H,3H-pyrazolo[3,4-d]pyrimidin-3-one (Compound 174)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-{6-[methyl(piperidin-4-yl)amino]pyridin-2-yl}-2-(prop-2-en-1-yl)-6-{[1-(propan-2-yl)-1H-pyrazol-4-yl]amino}-1H,2H,3H-pyrazolo[3,4-d]pyrimidin-3-one; trifluoroacetic acid (110 mg). Yield: 37 mg TFA salt (33%) as a powder.

### Example 176. Synthesis of 1-{6-[methyl(1-methylpiperidin-4-yl)amino]pyridin-2-yl}-6-{[1-(2-methylpropyl)-1H-pyrazol-4-yl]amino}-2-(prop-2-en-1-yl)-1H,2H,3H-pyrazolo[3,4-d]pyrimidin-3-one (Compound 175)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-{6-[methyl(piperidin-4-yl)amino]pyridin-2-yl}-6-{[1-(2-methylpropyl)-1H-pyrazol-4-yl]amino}-2-(prop-2-en-1-yl)-1H,2H,3H-pyrazolo[3,4-d]pyrimidin-3-one; trifluoroacetic acid (126 mg). Yield: 67 mg TFA salt (52%) as a powder.

### Example 177. Synthesis of 1-{6-[(3R)-1-azabicyclo[2.2.2]octan-3-yloxy]pyridin-2-yl}-6-[(4-chlorophenyl)amino]-2-(prop-2-en-1-yl)-1H,2H,3H-pyrazolo[3,4-d]pyrimidin-3-one (Compound 176)

This compound was made using a similar method as described in the synthesis of (R)-2-allyl-6-((1-methyl-1H-pyrazol-3-yl)amino)-1-(6-(quinuclidin-3-yloxy)pyridin-2-yl)-1,2-dihydro-3H-pyrazolo[3,4-d]pyrimidin-3-one using 1-{6-[(3R)-1-azabicyclo[2.2.2]octan-3-yloxy]pyridin-2-yl}-6-(methylsulfanyl)-2-(prop-2-en-1-yl)-1H,2H,3H-pyrazolo[3,4-d]pyrimidin-3-one (100 mg) and 4-chloroaniline (2 equiv.). Yield: 67 mg TFA salt (46%) as a yellow powder.

### Example 178. Synthesis of 2-allyl-1-[6-(4-piperidyloxy)-2-pyridyl]-6-(1-propyl-4-pyrazolylamino)-1,2-dihydro-3H-1,2,5,7-tetraazainden-3-one (Compound 179)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (123 mg) and 1-propyl-4-pyrazolylamine (48.9 mg). Yield: 12 mg TFA salt.

### Example 179. Synthesis of 1-{6-[methyl(piperidin-4-yl)amino]pyridin-2-yl}-6-[(1-methyl-1H-pyrazol-4-yl)amino]-2-(prop-2-en-1-yl)-1H,2H,3H-pyrazolo[3,4-d]pyrimidin-3-one (Compound 182)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using tert-butyl 4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate (200 mg) and 1-methyl-4-pyrazolamine (1 equiv.) to give the crude free base intermediate (227 mg) as a brown oil of which 13% was purified by prep-HPLC. Yield: 13.4 mg TFA salt (45%) as a powder.

### Example 180. Synthesis of 1-[6-(N-methyl-N-4-piperidylamino)-2-pyridyl]-2-allyl-6-[1-(2-fluoro-2-methylpropyl)-4-pyrazolylamino]-1,2-dihydro-3H-1,2,5,7-tetraazainden-3-one (Compound 183)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using tert-butyl 4-({6-[2-allyl-6-(methylthio)-3-oxo-1,2-dihydro-3H-1,2,5,7-tetraazainden-1-yl]-2-pyridyl}-N-methylamino)-1-piperidinecarboxylate (200 mg) and 1-(2-fluoro-2-methylpropyl)-4-pyrazolylamine (67.6 mg) followed by deprotection of the boc-group. Yield 14.5 mg (TFA salt).

### Example 181. Synthesis of 1-{6-[methyl(1-methylpiperidin-4-yl)amino]pyridin-2-yl}-6-[(1-methyl-1H-pyrazol-4-yl)amino]-2-(prop-2-en-1-yl)-1H,2H,3H-pyrazolo[3,4-d]pyrimidin-3-one (Compound 184)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using 1-{6-[methyl(piperidin-4-yl)amino]pyridin-2-yl}-6-[(1-methyl-1H-pyrazol-4-yl)amino]-2-(prop-2-en-1-yl)-1H,2H,3H-pyrazolo[3,4-d]pyrimidin-3-one (197 mg, crude). Yield: 50 mg TFA salt (20%) as a powder.

### Example 182. Synthesis of 6-{[1-(2-fluoro-2-methylpropyl)-1H-pyrazol-4-yl]amino}-1-{6-[methyl(1-methylpiperidin-4-yl)amino]pyridin-2-yl}-2-(prop-2-en-1-yl)-1H,2H,3H-pyrazolo[3,4-d]pyrimidin-3-one (Compound 185)

This compound was made using a similar method as described in the synthesis of 6-[(4-chlorophenyl)amino]-1-{6-[(1-methylpiperidin-4-yl)(propyl)amino]pyridin-2-yl}-2-(prop-2-en-1-yl)-1H,2H,3H-pyrazolo[3,4-d]pyrimidin-3-one using 6-{[1-(2-fluoro-2-methylpropyl)-1H-pyrazol-4-yl]amino}-1-{6-[methyl(piperidin-4-yl)amino]pyridin-2-yl}-2-(prop-2-en-1-yl)-1H,2H,3H-pyrazolo[3,4-d]pyrimidin-3-one (290 mg, crude). Yield: 112 mg TFA salt (31%) as a powder.

### Example 183. Synthesis of 1-{6-[(R)-1-methyl-3-piperidyloxy]-2-pyridyl}-2-allyl-6-(p-chlorophenylamino)-1,2-dihydro-3H-1,2,5,7-tetraazainden-3-one (Compound 187)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-{6-[(R)-3-piperidyloxy]-2-pyridyl}-2-allyl-6-(p-chlorophenylamino)-1,2-dihydro-3H-1,2,5,7-tetraazainden-3-one (25 mg). Yield 20 mg (TFA salt).

### Example 184. Synthesis of 6-[(1-methyl-1H-pyrazol-4-yl)amino]-1-(6-{[(trans)-8-methyl-8-azabicyclo[3.2.1]octan-3-yl]oxy}pyridin-2-yl)-2-(prop-2-en-1-yl)-1H,2H,3H-pyrazolo[3,4-d]pyrimidin-3-one (Compound 190) and 6-[(1-methyl-1H-pyrazol-4-yl)amino]-1-(6-{[(cis)-8-methyl-8-azabicyclo[3.2.1]octan-3-yl]oxy}pyridin-2-yl)-2-(prop-2-en-1-yl)-1H,2H,3H-pyrazolo[3,4-d]pyrimidin-3-one (Compound 189)

### tert-butyl 3-[(6-bromopyridin-2-yl)oxy]-8-azabicyclo[3.2.1]octane-8-carboxylate

This intermediate was made using a similar method as described in the synthesis tert-butyl 4-((6-bromopyridin-2-yl)oxy)piperidine-1-carboxylate using 2,6-dibromopyridine (2 g) and tert-butyl 3-hydroxy-8-azabicyclo[3.2.1]octane-8-carboxylate (1 equiv.). Yield: 2.4 g (71%) as a colourless oil. Mixture of diastereomers.

### tert-butyl 3-({6-[6-(methylsulfanyl)-3-oxo-2-(prop-2-en-1-yl)-1H,2H,3H-pyrazolo[3,4-d]pyrimidin-1-yl]pyridin-2-yl}oxy)-8-azabicyclo[3.2.1]octane-8-carboxylate

This intermediate was made using a similar method as described in the synthesis of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl 3-[(6-bromopyridin-2-yl)oxy]-8-azabicyclo[3.2.1]octane-8-carboxylate (850 mg). Purified by flash chromatography (SiO₂, 0-40% ethyl acetate in petroleum ether). Yield: 687 mg mixture of diastereomers (59%) as an orange oil.

### 1-(6-{8-azabicyclo[3.2.1]octan-3-yloxy}pyridin-2-yl)-6-[(1-methyl-1H-pyrazol-4-yl)amino]-2-(prop-2-en-1-yl)-1H,2H,3H-pyrazolo[3,4-d]pyrimidin-3-one

This compound was made using a similar method as described in the synthesis 2-allyl-6-((4-chlorophenyl)amino)-1-(6-(piperidin-4-yloxy)pyridin-2-yl)-1,2-dihydro-3H-pyrazolo[3,4-d]pyrimidin-3-one using tert-butyl 3-({6-[6-(methylsulfanyl)-3-oxo-2-(prop-2-en-1-yl)-1H,2H,3H-pyrazolo[3,4-d]pyrimidin-1-yl]pyridin-2-yl}oxy)-8-azabicyclo[3.2.1]octane-8-carboxylate (400 mg) and 1-methyl-1H-pyrazol-4-amine hydrochloride (1.1 equiv.). Yield: 437 mg (crude) mixture of diastereomers as a brown oil.

### 6-[(1-methyl-1H-pyrazol-4-yl)amino]-1-(6-{[(trans)-8-methyl-8-azabicyclo[3.2.1]octan-3-yl]oxy}pyridin-2-yl)-2-(prop-2-en-1-yl)-1H,2H,3H-pyrazolo[3,4-d]pyrimidin-3-one and 6-[(1-methyl-1H-pyrazol-4-yl)amino]-1-(6-{[(cis)-8-methyl-8-azabicyclo[3.2.1]octan-3-yl]oxy}pyridin-2-yl)-2-(prop-2-en-1-yl)-1H,2H,3H-pyrazolo[3,4-d]pyrimidin-3-one

These compounds were made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 1-(6-{8-azabicyclo[3.2.1]octan-3-yloxy}pyridin-2-yl)-6-[(1-methyl-1H-pyrazol-4-yl)amino]-2-(prop-2-en-1-yl)-1H,2H,3H-pyrazolo[3,4-d]pyrimidin-3-one (422 mg, crude, mixture of diastereomers). The diastereomers were separated by prep-HPLC on C18 (CH₃CN/H₂O+0.1% NH₃) to yield 27 mg of Compound 189 and 56 mg of Compound 190.

### Example 185. Synthesis of 2-allyl-6-(1-isopropyl-4-pyrazolylamino)-1-{6-(9-methyl-9-azabicyclo[3.3.1]non-3-yloxy)-2-pyridyl}-1,2-dihydro-3H-1,2,5,7-tetraazainden-3-one (Compound 193)

This compound was made using a similar method as described in the synthesis of allyl-6-(p-bromophenylamino)-1-[6-(1-methyl-4-piperidyloxy)-2-pyridyl]-1,2-dihydro-3H-1,2,5,7-tetraazainden-3-one. Yield 35 mg.

### Example 186. Synthesis of 6-[(2,6-dimethylpyridin-4-yl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 199)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using 6-[(2,6-dimethylpyridin-4-yl)amino]-1-[6-(piperidin-4-yloxy)pyridin-2-yl]-2-(prop-2-en-1-yl)-1H,2H,3H-pyrazolo[3,4-d]pyrimidin-3-one (crude). Yield: 67 mg TFA salt as a powder.

### Example 187. Synthesis of 6-{[2-methyl-6-(trifluoromethyl)pyridin-4-yl]amino}-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 202)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using 6-[(2,6-dimethylpyridin-4-yl)amino]-1-[6-(piperidin-4-yloxy)pyridin-2-yl]-2-(prop-2-en-1-yl)-1H,2H,3H-pyrazolo[3,4-d]pyrimidin-3-one (crude). Yield: 105 mg TFA salt as a powder.

### Example 188. Synthesis of 2-methyl-4-[(1-{6-[(1-methylpiperidin-4-yl)oxy]pyridin-2-yl}-3-oxo-2-(prop-2-en-1-yl)-1H,2H,3H-pyrazolo[3,4-d]pyrimidin-6-yl)amino]benzonitrile (Compound 204)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 2-methyl-4-({3-oxo-1-[6-(piperidin-4-yloxy)pyridin-2-yl]-2-(prop-2-en-1-yl)-1H,2H,3H-pyrazolo[3,4-d]pyrimidin-6-yl}amino)benzonitrile (crude). Yield: 28 mg TFA salt as a powder.

### Example 189. Synthesis of 6-[(3-fluoro-5-methylphenyl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 205)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 1-{6-[(1-methylpiperidin-4-yl)oxy]pyridin-2-yl}-6-(methylsulfanyl)-2-(prop-2-en-1-yl)-1H,2H,3H-pyrazolo[3,4-d]pyrimidin-3-one (100 mg) and 3-fluoro-5-methylaniline (1 equiv.). Yield: 47 mg TFA salt (32%) as a white powder.

### Example 190. Synthesis of 6-[(4-fluoro-3-methylphenyl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 206)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 1-{6-[(1-methylpiperidin-4-yl)oxy]pyridin-2-yl}-6-(methylsulfanyl)-2-(prop-2-en-1-yl)-1H,2H,3H-pyrazolo[3,4-d]pyrimidin-3-one (100 mg) and 4-fluoro-3-methylaniline (1 equiv.). Yield: 30 mg TFA salt (21%) as a powder.

### Example 191. Synthesis of 6-[(4-fluorophenyl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 207)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 1-{6-[(1-methylpiperidin-4-yl)oxy]pyridin-2-yl}-6-(methylsulfanyl)-2-(prop-2-en-1-yl)-1H,2H,3H-pyrazolo[3,4-d]pyrimidin-3-one (100 mg) and 4-fluoroaniline (1 equiv.). Yield: 30 mg TFA salt (21%) as a powder.

### Example 192. Synthesis of 2-allyl-6-(benzo[d]thiazol-5-ylamino)-1-(6-(piperidin-4-yloxy)pyridin-2-yl)-1,2-dihydro-3H-pyrazolo[3,4-d]pyrimidin-3-one (Compound 246) and 2-allyl-6-(benzo[d]thiazol-5-ylamino)-1-(6-(piperidin-4-yloxy)pyridin-2-yl)-1,2-dihydro-3H-pyrazolo[3,4-d]pyrimidin-3-one (Compound 209)

### tert-butyl 4-((6-(2-allyl-6-(benzo[d]thiazol-5-ylamino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (1.2 g, 2.262 mmol) in Acetic acid (20 mL) was added benzo[d]thiazol-5-amine (0.374 g, 2.488 mmol) at room temperature. The resultant reaction mixture was subjected to stirring at 25 °C for 16 h. The progress of the reaction was monitored by TLC. After completion of the reaction, the reaction mixture was directly concentrated under reduced pressure, and the residue was diluted with water and extracted with 10% methanol-DCM (50 mL×2). The combined organic layer was dried over Na₂SO₄, filtered, and concentrated under reduced pressure. The above crude was purified by flash column chromatography (silica gel, 100-200 mesh size) using ethyl acetate-hexane (50-60%) as an eluent to obtain tert-butyl 4-((6-(2-allyl-6-(benzo[d]thiazol-5-ylamino)-3-oxo-2,3-dihydro-1H-pyrazolo[3,4-d]pyrimidin-1-yl)pyridin-2-yl)oxy)piperidine-1-carboxylate (3, 1 g, 1.648 mmol, 72.9% yield) as a brown solid.

### 2-allyl-6-(benzo[d]thiazol-5-ylamino)-1-(6-(piperidin-4-yloxy)pyridin-2-yl)-1,2-dihydro-3H-pyrazolo[3,4-d]pyrimidin-3-one (Compound 246)

To a stirred solution of tert-butyl 4-((6-(2-allyl-6-(benzo[d]thiazol-5-ylamino)-3-oxo-2,3-dihydro-1H-pyrazolo[3,4-d]pyrimidin-1-yl)pyridin-2-yl)oxy)piperidine-1-carboxylate (1 g, 1.665 mmol) in 1,4-dioxane (5 mL), was added 4M HCl in 1,4-dioxane (2.0 mL) at 0 °C. The resulting reaction mixture was stirred at room temperature for 4 h. The progress of the reaction was monitored by TLC. After completion of the reaction, the reaction mixture was concentrated under reduced pressure. The resultant crude (300 mg) residue was purified by prep. HPLC X-Select C18 (19*250, 5 mL), Mobile phase A: 0.1 M FA in H2O, Mobile phase B: acetonitrile) to get 2-allyl-6-(benzo[d]thiazol-5-ylamino)-1-(6-(piperidin-4-yloxy)pyridin-2-yl)-1,2-dihydro-3H-pyrazolo[3,4-d]pyrimidin-3-one (Compound 246, 130 mg, 0.260 mmol, 15.60% yield) as an off white-solid. The remaining 600 mg was taken as such for next step without further purification.

### 2-allyl-6-(benzo[d]thiazol-5-ylamino)-1-(6-((1-methylpiperidin-4-yl)oxy)pyridin-2-yl)-1,2-dihydro-3H-pyrazolo[3,4-d]pyrimidin-3-one (Compound 209)

To a stirred solution of 2-allyl-6-(benzo[d]thiazol-5-ylamino)-1-(6-(piperidin-4-yloxy)pyridin-2-yl)-1,2-dihydro-3H-pyrazolo[3,4-d]pyrimidin-3-one (500 mg, 0.999 mmol) in THF (10 ml) was 20% aq. formaldehyde (405 mg, 4.99 mmol) was added at 25 °C. The reaction mixture was stirred at 25 °C for 30 min. After 30 min sodium triacetoxyborohydride (STAB 635 mg, 3.00 mmol) was added portion wise under inert atmosphere. After the complete addition of STAB, the reaction mixture was stirred to 25 °C and continued to stir for 1 h. The progress of the reaction was monitored by LCMS after completion of the reaction. The reaction mixture was quenched by 10% NaOH solution and extracted with 10% methanol-DCM. Organic layers were dried over sodium sulfate and concentrated over a vacuum to afford crude compound. Then crude compound was purified by prep HPLC. After prep, HPLC pure fractions were lyophilized to get 2-allyl-6-(benzo[d]thiazol-5-ylamino)-1-(6-((1-methylpiperidin-4-yl)oxy)pyridin-2-yl)-1,2-dihydro-3H-pyrazolo[3,4-d]pyrimidin-3-one (80 mg, 0.428 mmol, 17% yield) as an pale yellow solid.

### Example 193. Synthesis of 6-[(1,3-benzothiazol-6-yl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 212)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using 6-[(1,3-benzothiazol-6-yl)amino]-1-[6-(piperidin-4-yloxy)pyridin-2-yl]-2-(prop-2-en-1-yl)-1H,2H,3H-pyrazolo[3,4-d]pyrimidin-3-one (21 mg, crude). Yield: 19 mg TFA salt (80%) as a pale-yellow powder.

### Example 194. Synthesis of 6-[(1,2-benzoxazol-6-yl)amino]-1-[6-(piperidin-4-yloxy)pyridin-2-yl]-2-(prop-2-en-1-yl)-1H,2H,3H-pyrazolo[3,4-d]pyrimidin-3-one (Compound 214)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using tert-butyl 4-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate (150 mg) and 1,2-benzoxazol-6-amine (2 equiv.). Yield: 145 mg.

### Example 195. Synthesis of 2-allyl-1-(3-(methyl(1-methylpiperidin-4-yl)amino)phenyl)-6-((1-methyl-1H-indazol-5-yl)amino)-1,2-dihydro-3H-pyrazolo[3,4-d]pyrimidin-3-one (Compound 217)

### 2-allyl-6-(methylthio)-1-(3-nitrophenyl)-1,2-dihydro-3H-pyrazolo[3,4-d]pyrimidin-3-one

To a solution of 2-allyl-6-(methylthio)-1,2-dihydro-3H-pyrazolo[3,4-d]pyrimidin-3-one (1.2 g, 5.40 mmol) in dichloroethane (15 mL) was added 3-nitrophenyl boronic acid (1.352 g, 8.10 mmol), sodium carbonate (1.707 g, 16.20 mmol) and copper (II) acetate (0.49 g, 2.70 mmol) followed by pyridine (0.169 g, 1.080 mmol) and the mixture was stirred at room temperature and the temperature raised to 70 °C and stirred for 16 h. The progress of the reaction was monitored by TLC and LCMS. After completion of the reaction, the reaction mass was filtered through celite and washed with 10% MeOH in DCM (2×100 mL). The combined organic layers were dried over anhydrous Na₂SO₄, evaporated under reduced pressure to give the crude compound which was purified by column chromatography (silica mesh 100-200 at eluent of 70-100% Ethyl acetate and hexane) to get the pure compound 2-allyl-6-(methylthio)-1-(3-nitrophenyl)-1,2-dihydro-3H-pyrazolo[3,4-d]pyrimidin-3-one (700 mg, 1.794 mmol, 33.2% yield) as a white solid.

### 2-allyl-6-(methylsulfonyl)-1-(3-nitrophenyl)-1,2-dihydro-3H-pyrazolo[3,4-d]pyrimidin-3-one

To a stirred solution of 2-allyl-6-(methylthio)-1-(3-nitrophenyl)-1,2-dihydro-3H-pyrazolo[3,4-d]pyrimidin-3-one (50 mg, 0.146 mmol) in DCM (10 ml) was added mCPBA (53.7 mg, 0.218 mmol) at room temperature under inert atmosphere, and the mixture was stirred for 2 h. The progress of the reaction was monitored by TLC and UPLC. After completion of the reaction, the reaction mixture was quenched with aq. 10% sodium bicarbonate solution (10 mL) and extracted with 10% MeOH in DCM (2×50 mL). The combined organic layers were washed with brine (100 mL), dried over anhydrous sodium sulfate, filtered, and concentrated under reduced pressure to give the crude compound 2-allyl-6-(methylsulfonyl)-1-(3-nitrophenyl)-1,2-dihydro-3H-pyrazolo[3,4-d]pyrimidin-3-one (30 mg, 0.024 mmol, 16.47% yield) as an off-white solid. This crude compound was taken as such for the next step without further purification.

### 2-allyl-6-((1-methyl-1H-indazol-5-yl)amino)-1-(3-nitrophenyl)-1,2-dihydro-3H-pyrazolo[3,4-d]pyrimidin-3-one

To a stirred solution of 2-allyl-6-(methylthio)-1-(3-nitrophenyl)-1,2-dihydro-3H-pyrazolo[3,4-d]pyrimidin-3-one (0.7 g, 2.039 mmol) in AcOH (5 mL) was added 1-methyl-1H-indazol-5-amine (0.3 g, 2.039 mmol) and the mixture was allowed to stir for 16 h at room temperature. The progress of the reaction was monitored by TLC and UPLC. After completion of the reaction, solvents were evaporated under reduced pressure and the residue obtained was quenched with 10% aq. sodium bicarbonate solution (100 mL) and extracted with ethyl acetate (2×300 mL). The combined organic extracts were washed with brine (100 mL), dried over anhydrous sodium sulfate, filtered and concentrated under reduced pressure to give the crude compound which was purified by column chromatography (silica mesh 100-200 at eluent of 10-20% ethyl acetate and hexane) to get the pure compound 2-allyl-6-((1-methyl-1H-indazol-5-yl)amino)-1-(3-nitrophenyl)-1,2-dihydro-3H-pyrazolo[3,4-d]pyrimidin-3-one (600 mg, 1.221 mmol, 66.51% yield) as a yellow solid.

### tert-butyl(2-allyl-1-(3-nitrophenyl)-3-oxo-2,3-dihydro-1H-pyrazolo[3,4-d]pyrimidin-6-yl)(1-methyl-1H-indazol-5-yl) carbamate

To a stirred solution of 2-allyl-6-((1-methyl-1H-indazol-5-yl)amino)-1-(3-nitrophenyl)-1,2-dihydro-3H-pyrazolo[3,4-d]pyrimidin-3-one (0.2 g, 0.452 mmol) in DCM (5 mL) was added TEA (0.452 mmol) and Boc anhydride (0.148 g, 0.678 mmol) at 0 °C under inert atmosphere and the resulting mixture was allowed to stir for 16 h at room temperature. The progress of the reaction was monitored by TLC and UPLC. After completion of the reaction, the reaction mixture was diluted with water (100 mL) and extracted with ethyl acetate (2×200 mL). The combined organic layers were washed with brine (100 mL), dried over anhydrous sodium sulfate, filtered and concentrated under reduced pressure to give tert-butyl(2-allyl-1-(3-nitrophenyl)-3-oxo-2,3-dihydro-1H-pyrazolo[3,4-d]pyrimidin-6-yl)(1-methyl-1H-indazol-5-yl) carbamate (250 mg, 0.424 mmol, 94% yield) as a white solid.

### tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-nitrophenyl)-3-oxo-2,3-dihydro-1H-pyrazolo[3,4-d]pyrimidin-6-yl)(1-methyl-1H-indazol-5-yl) carbamate (250 mg, 0.461 mmol) in EtOH (3.0 mL) and water (10 mL) was added Iron (257 mg, 4.61 mmol) and NH₄Cl (246 mg, 4.61 mmol) and the resulting mixture was allowed to stir for 16 h. The progress of the reaction was monitored by TLC and UPLC. After completion of the reaction, the reaction mixture was diluted with water (100 mL) and extracted with ethyl acetate (2×30 mL). The combined organic layers was washed with brine, dried over anhydrous Na₂SO₄, filtered, and concentrated under reduced pressure to give the crude compound which was purified by column chromatography (silica gel mesh 100-200, at eluent of 50-80% ethyl acetate in hexane) to give tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate (0.150 g, 0.234 mmol, 50.8% yield) as a brown solid compound.

### tert-butyl(2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-aminophenyl)-3-oxo-2,3-dihydro-1H-pyrazolo[3,4-d]pyrimidin-6-yl)(1-methyl-1H-indazol-5-yl) carbamate (0.15 g, 0.293 mmol) and 1-methylpiperidin-4-one (0.033 g, 0.293 mmol) in Dichloroethane (5 mL) was added AcOH (1 mL) under inert atmosphere. The mixture was stirred for 4 h at room temperature and then cooled to 0 °C. STAB (0.311 g, 0.293 mmol) was added and the mixture allowed to stir for 16 h. The progress of the reaction was monitored by TLC and UPLC. After completion of the reaction, the reaction mixture was diluted with water (100 mL) and extracted with ethyl acetate (2×100 mL). The combined organic layers was washed with brine (100 mL), dried over anhydrous sodium sulfate, filtered, and concentrated under reduced pressure to give the crude compound which was purified by flash column chromatography (silica mesh 100-200 at eluent of 0-20% MeOH and DCM) to give tert-butyl (2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (140 mg, 0.204 mmol, 69.8% yield) as a yellow solid compound.

### tert-butyl(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate

To a stirred solution of tert-butyl(2-allyl-1-(3-((1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (0.130 g, 0.213 mmol) in THF (2 mL) was added NaH (0.012 g, 0.533 mmol) and allowed to stir for 30 min at 0 °C under inert atmosphere. Mel (0.030 g, 0.213 mmol) was added and the mixture was allowed to stir for 16 h at room temperature. The progress of the reaction was monitored by TLC and UPLC. After completion of the reaction, the reaction mixture was diluted with water (100 mL) and extracted with DCM (2×100 mL). The combined organic layers was washed with brine (100 mL), dried over anhydrous sodium sulfate, filtered, and concentrated under reduced pressure to give the crude compound tert-butyl(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (120 mg) as a yellow solid. The crude compound was taken as such for the next step without purification.

### 2-allyl-1-(3-(methyl(1-methylpiperidin-4-yl)amino)phenyl)-6-((1-methyl-1H-indazol-5-yl)amino)-1,2-dihydro-3H-pyrazolo[3,4-d]pyrimidin-3-one

To a stirred solution of tert-butyl(2-allyl-1-(3-(methyl(1-methylpiperidin-4-yl)amino)phenyl)-3-oxo-2,3-dihydro-1H-pyrazolo[3,4-d]pyrimidin-6-yl)(1-methyl-1H-indazol-5-yl) carbamate (90 mg, 0.144 mmol) in DCM (5 mL) was added 4M HCl in 1,4dioxane (0.3 mL, 1.200 mmol) and the mixture was stirred at room temperature for 16 h. The progress of the reaction was monitored by TLC and UPLC. After completion of the reaction, the reaction mixture was concentrated under reduced pressure to get the crude compound which was purified by prep-HPLC (Column: X-select CSH C18, Mobile Phase A: 0.1% Formic acid in water, Mobile Phase B: acetonitrile, Flowrate: 15.0 mL/Min, Rt-10.8) to give 2-allyl-1-(3-(methyl(1-methylpiperidin-4-yl)amino)phenyl)-6-((1-methyl-1H-indazol-5-yl)amino)-1,2-dihydro-3H-pyrazolo[3,4-d]pyrimidin-3-one (7.7 mg, 0.015 mmol, 10.09% yield) as an off-white solid.

### Example 196. Synthesis of 2-allyl-6-((1-methyl-1H-indazol-5-yl)amino)-1-(3-((1-methylpiperidin-4-yl)oxy)phenyl)-1,2-dihydro-3H-pyrazolo[3,4-d]pyrimidin-3-one (Compound 218)

To a stirred solution of 2-allyl-6-((1-methyl-1H-indazol-5-yl)amino)-1-(3-(piperidin-4-yloxy)phenyl)-1,2-dihydro-3H-pyrazolo[3,4-d]pyrimidin-3-one (210 mg, 0.423 mmol) in THF (5 mL) was added 37% aq. formaldehyde (172 mg, 2.114 mmol) at 25 °C, and the mixture was stirred for 10 min, then STAB (269 mg, 1.269 mmol) was added portion wise. After the complete addition of STAB, the reaction mixture was stirred at 25 °C for 16 h. The progress of the reaction was monitored by UPLC. After completion of the reaction, the reaction mixture was quenched with TFA, followed by Ammonia in MeOH. The reaction mixture was diluted with water and extracted with 10% MeOH in DCM. The combined organic extracts were washed with aq. sodium bicarbonate, dried over anhydrous Na₂SO₄, filtered and concentrated under reduced pressure to afford crude compound which was purified by Prep-HPLC (FA method) to give 2-allyl-6-((1-methyl-1H-indazol-5-yl)amino)-1-(3-((1-methylpiperidin-4-yl)oxy)phenyl)-1,2-dihydro-3H-pyrazolo[3,4-d]pyrimidin-3-one (21.22 mg, 0.040 mmol, 9.53% yield) as a white solid.

### Example 197. Synthesis of 2-allyl-6-((4-(2-fluoroethoxy)phenyl)amino)-1-(6-(piperidin-4-yloxy)pyridin-2-yl)-1,2-dihydro-3H-pyrazolo[3,4-d]pyrimidin-3-one (Compound 233) and 2-allyl-6-((4-(2-fluoroethoxy)phenyl)amino)-1-(6-((1-methylpiperidin-4-yl)oxy)pyridin-2-yl)-1,2-dihydro-3H-pyrazolo[3,4-d]pyrimidin-3-one (Compound 234)

### 1-(2-fluoroethoxy)-4-nitrobenzene

To a stirred solution of 4-nitrophenol (1.5 g, 10.78 mmol) in DMF (10 ml) was added K₂CO₃ (4.47 g, 32.3 mmol) followed by the addition of 1-fluoro-2-iodoethane (2, 1.876 g, 10.78 mmol) at 0 °C under inert atmosphere. The mixture was stirred at rt for 16 h. The progress of the reaction was monitored by TLC and LCMS. After completion of the reaction, water (20 mL) was added and the resulting solid was filtered and washed with n-hexane to get the pure compound 1-(2-fluoroethoxy)-4-nitrobenzene (3, 1.5 g, 8.10 mmol, 75% yield) as a white solid.

### 4-(2-fluoroethoxy)aniline

To a stirred solution of 1-(2-fluoroethoxy)-4-nitrobenzene (1 g, 5.40 mmol) in MeOH (10 ml) was added 10% Pd/C (0.575 g, 0.540 mmol) at rt under inert atmosphere. The mixture was stirred under H₂ bladder pressure for 16 h. The progress of the reaction was monitored by TLC and LCMS. After completion of the reaction, the reaction mass was filtered through celite and washed with MeOH (2×100 mL). The combined organic layers was collected and solvents evaporated under reduced pressure to give 4-(2-fluoroethoxy)aniline (850 mg, 4.82 mmol, 89% yield) as a brown colored solid.

### tert-butyl 4-((6-(2-allyl-6-((4-(2-fluoroethoxy)phenyl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (1 g, 1.885 mmol) in AcOH (12 ml) was added 4-(2-fluoroethoxy)aniline (0.439 g, 2.83 mmol) at rt under inert atmosphere. The mixture was stirred at rt for 16 h. The progress of the reaction was monitored by TLC and LCMS. After completion of the reaction, the solvent was evaporated under reduced pressure and the residue was quenched with aq. NaHCO₃ (50 mL) and then extracted with EA (2×100 mL). The combined organic layers were dried over anhydrous Na₂SO₄, evaporated under reduced pressure to give the crude compound which was purified by column chromatography (SiO₂/230-400 mesh; ~50-80% EtOAc/n-hexane) to give tert-butyl 4-((6-(2-allyl-6-((4-(2-fluoroethoxy)phenyl)amino)-3-oxo-2,3-dihydro-1H-pyrazolo[3,4-d]pyrimidin-1-yl)pyridin-2-yl)oxy)piperidine-1-carboxylate (500 mg, 0.793 mmol, 42.1% yield) as an off-white solid.

### 2-allyl-6-((4-(2-fluoroethoxy)phenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-(2-fluoroethoxy)phenyl)amino)-3-oxo-2,3-dihydro-1H-pyrazolo[3,4-d]pyrimidin-1-yl)pyridin-2-yl)oxy)piperidine-1-carboxylate (500 mg, 0.826 mmol) in 1,4-dioxane (4 ml) was added 4M HCl in dioxane (1 ml, 4.00 mmol) at 0 °C under inert atmosphere, and the mixture was then stirred at rt for 16 h. The progress of the reaction was monitored by TLC and LCMS. After completion of the reaction, the solvent was evaporated under reduced pressure. The residue was washed with n-hexane to get the compound 2-allyl-6-((4-(2-fluoroethoxy)phenyl)amino)-1-(6-(piperidin-4-yloxy)pyridin-2-yl)-1,2-dihydro-3H-pyrazolo[3,4-d]pyrimidin-3-one (400 mg, 0.792 mmol, 95.84% yield) as an off-white solid. Fifty mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to get the pure compound.

### 2-allyl-6-((4-(2-fluoroethoxy)phenyl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((4-(2-fluoroethoxy)phenyl)amino)-1-(6-(piperidin-4-yloxy)pyridin-2-yl)-1,2-dihydro-3H-pyrazolo[3,4-d]pyrimidin-3-one (400 mg, 0.791 mmol) in THF (5 ml) was added 37% formaldehyde (0.4 mL, 3.96 mmol) followed by the addition of STAB (503 mg, 2.374 mmol) portion wise. The reaction mixture was stirred at 25 °C for 1 h. The progress of the reaction was monitored by TLC and LCMS. After completion of the reaction, the reaction mixture was quenched with NH₃ in MeOH followed by TFA at rt and extracted with 10% MeOH in DCM (2×100 mL). The combined organic layers were dried over anhydrous Na₂SO₄. The solvent was evaporated under reduced pressure to give the crude compound which was purified by prep HPLC (Column: X-select CSH C18, Mobile Phase A: Ammonium acetate in water, Mobile Phase B: acetonitrile, Flowrate: 15.0 mL/Min, Rt-10.8) to give 2-allyl-6-((4-(2-fluoroethoxy)phenyl)amino)-1-(6-((1-methylpiperidin-4-yl)oxy)pyridin-2-yl)-1,2-dihydro-3H-pyrazolo[3,4-d]pyrimidin-3-one (143 mg, 0.271 mmol, 34.3% yield) as white solid.

### Example 198. Synthesis of 2-ethyl-6-((1-methyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one (Compound 235)

2-Ethyl-1-(6-fluoropyridin-2-yl)-6-(methylthio)-1,2-dihydro-3H-pyrazolo[3,4-d]pyrimidin-3-one

A stirred solution of 2-ethyl-6-(methylthio)-1,2-dihydro-3H-pyrazolo[3,4-d]pyrimidin-3-one (1 g, 4.76 mmol), 2-bromo-6-fluoropyridine (0.837 g, 4.76 mmol), trans-N,N-Dimethylethylenediamine (0.397 g, 4.50 mmol) and potassium carbonate (1.972 g, 14.27 mmol) in 1,4-dioxane (4 ml), was degassed for 5 min, then copper (I) iodide (0.904 g, 4.76 mmol) was added at 25 °C and the resulting mixture was stirred at 100 °C for 2 h. The progress of the reaction was monitored by TLC. After completion of the reaction, the reaction mixture was filtered on celite pad and washed with ethyl acetate (250 mL). The filtrate was evaporated under reduced pressure. The resulting crude material was purified by flash chromatography (SiO₂/100-200 mesh; 20-30% Ethyl acetate-hexane) to afford 2-ethyl-1-(6-fluoropyridin-2-yl)-6-(methylthio)-1,2-dihydro-3H-pyrazolo[3,4-d]pyrimidin-3-one (1 g, 3.28 mmol, 68.9% yield) as a pale brown solid.

### 2-Ethyl-1-(6-fluoropyridin-2-yl)-6-(methylsulfonyl)-1,2-dihydro-3H-pyrazolo[3,4-d]pyrimidin-3-one

To a stirred solution of 2-ethyl-1-(6-fluoropyridin-2-yl)-6-(methylthio)-1,2-dihydro-3H-pyrazolo[3,4-d]pyrimidin-3-one (700 mg, 2.293 mmol) in dichloromethane (30 ml) was added mCPBA (1187 mg, 4.81 mmol) at 0 °C and the resulting mixture stirred at 25 °C for 2 h. The progress of the reaction was monitored by TLC. After completion of the reaction, the reaction mixture was diluted with DCM (300 mL) and washed with sodium bicarbonate solution (2×150 mL). The combined organic layer was dried over Na₂SO₄, filtered and concentrated under reduced pressure to afford the crude 2-ethyl-1-(6-fluoropyridin-2-yl)-6-(methylsulfonyl)-1,2-dihydro-3H-pyrazolo[3,4-d]pyrimidin-3-one (600 mg, 1.138 mmol, 49.7% yield) as pale yellow solid.

### 2-ethyl-1-(6-fluoropyridin-2-yl)-6-((1-methyl-1H-benzo[d]imidazol-5-yl)amino)-1,2-dihydro-3H-pyrazolo[3,4-d]pyrimidin-3-one

To a stirred solution of 2-ethyl-1-(6-fluoropyridin-2-yl)-6-(methylsulfonyl)-1,2-dihydro-3H-pyrazolo[3,4-d]pyrimidin-3-one (650 mg, 1.927 mmol) in acetic acid (5 mL) was added 1-methyl-1H-benzo[d]imidazol-5-amine (312 mg, 2.120 mmol) at room temperature. The resulting reaction mixture was stirred at rt for 16 h. The progress of the reaction was monitored by TLC. After completion of the reaction, the reaction mixture was concentrated under reduced pressure. The residue was basified with sodium bicarbonate and extracted with ethyl acetate (10 mL×2). The combined organic layer was dried over Na₂SO₄, filtered, and concentrated under reduced pressure to afford crude material that was purified by flash chromatography (SiO₂/100-200 mesh; 10-15% methanol-DCM) to afford 2-ethyl-1-(6-fluoropyridin-2-yl)-6-((1-methyl-1H-benzo[d]imidazol-5-yl)amino)-1,2-dihydro-3H-pyrazolo[3,4-d]pyrimidin-3-one (550 mg, 1.238 mmol, 64.2% yield) as an off-white solid.

### 2-ethyl-6-((1-methyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one

NaH (60% in oil) (90.6 mg, 2.078 mmol) was added to a stirred solution of 1-methylpiperidin-4-ol (170 mg, 0.720 mmol) in Tetrahydrofuran (5 ml) at RT and the mixture was then stirred at 60 °C for 1 h. 2-ethyl-1-(6-fluoropyridin-2-yl)-6-((1-methyl-1H-benzo[d]imidazol-5-yl)amino)-1,2-dihydro-3H-pyrazolo[3,4-d]pyrimidin-3-one (400 mg, 0.990 mmol) was added and the mixture stirred at 60 °C for 16 h. The progress of the reaction was monitored by TLC. After completion of the reaction, the reaction mixture was diluted with ice water (150 mL) and extracted with 10% methanol-DCM (2×100 mL). The combined organic layer was dried over Na₂SO₄, filtered, and concentrated under reduced pressure. The resulting crude material was purified by prep. HPLC (Shimpak C18 (250×19 mm), Mobile phase A: 10 MM ABC in H₂O, Mobile phase B: acetonitrile) to give 2-ethyl-6-((1-methyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one (75 mg, 0.149 mmol, 30.1% yield) as an off white solid.

### Example 199. Synthesis of 2-allyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (Compound 236)

### tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (2.7 g, 5.42 mmol) in DCM (30 ml), mCPBA (1.869 g, 10.83 mmol) was added portion wise in reaction mixture at 0 °C. The reaction mixture was stirred at rt for 2 h. The reaction progress was monitored by TLC. The reaction mixture was quenched with 10% sodium bicarbonate solution in water (50 mL) and extracted with DCM (250 mL). The organic layer was washed with brine water (250 ml), and dried over Na₂SO₄, filtered, and concentrated under reduced pressure to give tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (2.5 g, 3.58 mmol, 66.1% yield) as a yellow gum. The crude compound was taken as such for the next step without further purification.

### tert-butyl 4-((6-(2-allyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (500 mg, 0.942 mmol) in acetic acid (10 ml), 1-methyl-1H-indol-5-amine (138 mg, 0.942 mmol) was added in the reaction mixture and stirred at RT for 6 h. The progress of the reaction was monitored by TLC. The reaction mixture was concentrated under vacuum to afford crude compound. The crude product was purified by flash column chromatography (silica-gel, mesh size 60-120) using ethyl acetate-hexane (55 to 60%) as an eluent to obtain tert-butyl 4-((6-(2-allyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (600 mg, 0.674 mmol) as an brown solid.

### 2-allyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (400 mg, 0.670 mmol) in DCM (10 ml), methane sulfonic acid (322 mg, 3.35 mmol) was added in the reaction mixture and stirred at RT for 6 h. The progress of the reaction was monitored by TLC. After completion of the reaction, the reaction mixture was concentrated under vacuum to afford crude compound. The crude compound was neutralized by aqueous sodium bicarbonate solution (50 ml) and extracted with 10% methanol-DCM. Organic layers were dried over sodium sulfate and concentrated under vacuum to afford crude compound. The crude compound was purified by prep-HPLC (Ammonium acetate method). Pure fractions were collected and lyophilized to give 2-allyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (23 mg, 0.045 mmol, 6.74% yield) as an off white solid.

### Example 200. Synthesis of 2-allyl-6-((6-methylpyridin-3-yl)amino)-1-(6-(piperidin-4-yloxy)pyridin-2-yl)-1,2-dihydro-3H-pyrazolo[3,4-d]pyrimidin-3-one (Compound 237)

### tert-butyl 4-((6-(2-allyl-6-((6-methylpyridin-3-yl)amino)-3-oxo-2,3-dihydro-1H-pyrazolo[3,4-d]pyrimidin-1-yl)pyridin-2-yl)oxy)piperidine-1-carboxylate

A stirred mixture of tert-butyl 4-((6-(2-allyl-6-amino-3-oxo-2,3-dihydro-1H-pyrazolo[3,4-d]pyrimidin-1-yl)pyridin-2-yl)oxy)piperidine-1-carboxylate (300 mg, 0.642 mmol) and potassium carbonate (310 mg, 2.246 mmol) in dioxane (8 ml) was degassed for 20 min at rt under inert atmosphere, then was added 5-bromo-2-methylpyridine (143 mg, 0.834 mmol) and stirred at 100 °C for 16 h. The progress of the reaction was monitored by UPLC; after completion of the reaction, the reaction mixture was diluted with DCM and filtered through celite pad. The collected filtrate was concentrated under reduced pressure. The crude was purified by flash column chromatography (silica gel, 100-200 mesh size) using ethyl acetate-pet ether (30%-40%) eluent to obtain tert-butyl 4-((6-(2-allyl-6-((6-methylpyridin-3-yl)amino)-3-oxo-2,3-dihydro-1H-pyrazolo[3,4-d]pyrimidin-1-yl)pyridin-2-yl)oxy)piperidine-1-carboxylate (350 mg, 0.601 mmol, 94% yield) as an off white solid.

### 2-allyl-6-((6-methylpyridin-3-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((6-methylpyridin-3-yl)amino)-3-oxo-2,3-dihydro-1H-pyrazolo[3,4-d]pyrimidin-1-yl)pyridin-2-yl)oxy)piperidine-1-carboxylate (150 mg, 0.269 mmol) in 1,4-dioxane (5 mL), was added 4M HCl in 1,4-dioxane (2.0 mL) at 0 °C. The resulting reaction mixture was stirred at room temperature for 4 h. The reaction was monitored by TLC. After completion of the reaction, the reaction mixture was concentrated under reduced pressure. The resultant crude residue was purified by prep. HPLC X-Select C18 (19*250, 5 mL), Mobile phase A: 10 MM AA in H2O, Mobile phase B: acetonitrile) to give 2-allyl-6-((6-methylpyridin-3-yl)amino)-1-(6-(piperidin-4-yloxy)pyridin-2-yl)-1,2-dihydro-3H-pyrazolo[3,4-d]pyrimidin-3-one (33 mg, 0.072 mmol, 26.8% yield) as an off white-solid.

### Example 201. Synthesis of 6-((1H-indazol-5-yl)amino)-2-allyl-1-(6-((1-methylpiperidin-4-yl)oxy)pyridin-2-yl)-1,2-dihydro-3H-pyrazolo[3,4-d]pyrimidin-3-one (Compound 238)

### 2-allyl-1-(6-fluoropyridin-2-yl)-6-(methylsulfonyl)-1,2-dihydro-3H-pyrazolo[3,4-d]pyrimidin-3-one

To a stirred solution of 2-allyl-1-(6-fluoropyridin-2-yl)-6-(methylthio)-1,2-dihydro-3H-pyrazolo[3,4-d]pyrimidin-3-one (1 g, 3.15 mmol) in DCM (10 ml) was added m-CPBA (1.679 g, 6.62 mmol) at 0 °C under inert atmosphere. The resulting reaction mixture was stirred at room temperature for 2 h. The progress of the reaction was monitored by TLC and LCMS. After completion of the reaction, the reaction mixture was quenched with sodium bicarbonate and extracted with DCM to give crude 2-allyl-1-(6-fluoropyridin-2-yl)-6-(methylsulfonyl)-1,2-dihydro-3H-pyrazolo[3,4-d]pyrimidin-3-one (1 g, 2.86 mmol, 91% yield). This crude compound was taken for the next step without any further purification.

### 6-((1H-indazol-5-yl)amino)-2-allyl-1-(6-fluoropyridin-2-yl)-1,2-dihydro-3H-pyrazolo[3,4-d]pyrimidin-3-one

To a stirred solution of 2-allyl-1-(6-fluoropyridin-2-yl)-6-(methylsulfonyl)-1,2-dihydro-3H-pyrazolo[3,4-d]pyrimidin-3-one (1 g, 2.86 mmol) in AcOH (5 ml) was added 1H-indazol-5-amine (0.572 g, 4.29 mmol) at room temperature under inert atmosphere. The resultant reaction mixture was stirred at 25 °C for 16 h. The progress of the reaction was monitored by TLC and LCMS. After completion of the reaction, the reaction mixture was diluted with water and extracted with 10% MeOH in DCM (2×50 mL). The combined organic layers were dried over Na₂SO₄, filtered, and concentrated under reduced pressure to give crude compound which was purified by column chromatography (SiO₂/230-400 mesh; 5-20% MeOH-DCM) to give 6-((1H-indazol-5-yl)amino)-2-allyl-1-(6-fluoropyridin-2-yl)-1,2-dihydro-3H-pyrazolo[3,4-d]pyrimidin-3-one (600 mg, 1.491 mmol, 52.1% yield) as a brown gummy solid.

### tert-butyl 5-((2-allyl-1-(6-fluoropyridin-2-yl)-3-oxo-2,3-dihydro-1H-pyrazolo[3,4-d]pyrimidin-6-yl)amino)-1H-indazole-1-carboxylate

To a stirred solution of 6-((1H-indazol-5-yl)amino)-2-allyl-1-(6-fluoropyridin-2-yl)-1,2-dihydro-3H-pyrazolo[3,4-d]pyrimidin-3-one (400 mg, 0.994 mmol) in DCM (10 ml) was added TEA (0.693 ml, 4.97 mmol) followed by the addition of DMAP (24.29 mg, 0.199 mmol) and Boc-anhydride (0.277 ml, 1.193 mmol) at room temperature under inert atmosphere. Then, the resultant reaction mixture was stirred at 25 °C for 16 h. The progress of the reaction was monitored by TLC and LCMS. After completion of the reaction, water was added (40 mL) to the reaction mixture and it was extracted with EtOAc (2×100 mL). The organic layer was concentrated under reduced pressure to give crude compound which was purified by flash column chromatography (SiO₂/230-400 mesh; 50-100% ethyl acetate-pet ether) to give tert-butyl 5-((2-allyl-1-(6-fluoropyridin-2-yl)-3-oxo-2,3-dihydro-1H-pyrazolo[3,4-d]pyrimidin-6-yl)amino)-1H-indazole-1-carboxylate (400 mg, 0.796 mmol, 80% yield) as an off-white solid.

### tert-butyl 5-((2-allyl-1-(6-((1-methylpiperidin-4-yl)oxy)pyridin-2-yl)-3-oxo-2,3-dihydro-1H-pyrazolo[3,4-d]pyrimidin-6-yl)amino)-1H-indazole-1-carboxylate

To a stirred solution of 1-methylpiperidin-4-ol (241 mg, 2.090 mmol) in THF (2 ml) was added NaH (84 mg, 2.090 mmol) at 0 °C and the mixture was stirred at 60 °C for 1 h. Then was added tert-butyl 5-((2-allyl-1-(6-fluoropyridin-2-yl)-3-oxo-2,3-dihydro-1H-pyrazolo[3,4-d]pyrimidin-6-yl)amino)-1H-indazole-1-carboxylate (350 mg, 0.697 mmol) and the reaction mixture was stirred at room temperature for 16 h. The progress of the reaction was monitored by TLC and LCMS. After completion of the reaction, the reaction mixture was quenched with ice-cold water, and the solid formed was filtered and dried under vacuum to give tert-butyl 5-((2-allyl-1-(6-((1-methylpiperidin-4-yl)oxy)pyridin-2-yl)-3-oxo-2,3-dihydro-1H-pyrazolo[3,4-d]pyrimidin-6-yl)amino)-1H-indazole-1-carboxylate (350 mg, 0.586 mmol, 84% yield) as an off-white solid. This compound, as such, was taken for the next step without any further purification.

### 6-((1H-indazol-5-yl)amino)-2-allyl-1-(6-((1-methylpiperidin-4-yl)oxy)pyridin-2-yl)-1,2-dihydro-3H-pyrazolo[3,4-d]pyrimidin-3-one

To a stirred solution of tert-butyl 5-((2-allyl-1-(6-((1-methylpiperidin-4-yl)oxy)pyridin-2-yl)-3-oxo-2,3-dihydro-1H-pyrazolo[3,4-d]pyrimidin-6-yl)amino)-1H-indazole-1-carboxylate (350 mg, 0.586 mmol) in DCM (15 ml) was added 4M HCl in dioxane (3 ml, 12 mmol) at 0 °C under inert atmosphere. The resulting reaction mixture was stirred at room temperature for 16 h. The progress of the reaction was monitored by TLC and LCMS. After completion of the reaction, the reaction mixture was concentrated under reduced pressure. The resultant crude residue was washed with n-hexane to give crude compound which was purified by Prep HPLC (X Select C18, 19×250, Mobile phase A: 0.1% AA in H2O, Mobile phase B: acetonitrile) to give 6-((1H-indazol-5-yl)amino)-2-allyl-1-(6-((1-methylpiperidin-4-yl)oxy)pyridin-2-yl)-1,2-dihydro-3H-pyrazolo[3,4-d]pyrimidin-3-one (77 mg, 0.128 mmol, 26% yield) as an off-white solid.

### Example 202. Synthesis of 2-allyl-1-[6-(4-piperidyloxy)-2-pyridyl]-6-(7-quinolylamino)-1,2-dihydro-3H-1,2,5,7-tetraazainden-3-one (Compound 243)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one. Yield 50 mg.

### Example 203. Synthesis of 2-allyl-6-((4-fluorophenyl)amino)-1-(2-((1-methylpiperidin-4-yl)oxy)pyrimidin-4-yl)-1,2-dihydro-3H-pyrazolo[3,4-d]pyrimidin-3-one (Compound 348) and 2-allyl-6-((4-fluorophenyl)amino)-1-(2-((piperidin-4-yl)oxy)pyrimidin-4-yl)-1,2-dihydro-3H-pyrazolo[3,4-d]pyrimidin-3-one (Compound 247)

### tert-butyl 4-((4-(2-allyl-6-((4-fluorophenyl)amino)-3-oxo-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (200 mg, 0.376 mmol) in acetic acid (2 ml) was added 4-fluoroaniline (62.7 mg, 0.564 mmol) at rt under inert atmosphere. The mixture was then stirred at rt for 16 h. The progress of the reaction was monitored by TLC and LCMS. After completion of the reaction, the solvent was evaporated under reduced pressure. To the residue was added aq. NaHCO₃ (10 mL), at which a appeared solid was filtered and washed with MTBE and n-hexane to give tert-butyl 4-((4-(2-allyl-6-((4-fluorophenyl)amino)-3-oxo-2,3-dihydro-1H-pyrazolo[3,4-d]pyrimidin-1-yl)pyrimidin-2-yl)oxy)piperidine-1-carboxylate (160 mg, 0.270 mmol, 71.8% yield) as an off-white solid.

### 2-allyl-6-((4-fluorophenyl)amino)-1-(2-(piperidin-4-yloxy)pyrimidin-4-yl)-1,2-dihydro-3H-pyrazolo[3,4-d]pyrimidin-3-one

To a stirred solution of tert-butyl 4-((4-(2-allyl-6-((4-fluorophenyl)amino)-3-oxo-2,3-dihydro-1H-pyrazolo[3,4-d]pyrimidin-1-yl)pyrimidin-2-yl)oxy)piperidine-1-carboxylate (300 mg, 0.533 mmol) in dioxane (3 ml) was added 4M HCl in dioxane (2 ml, 8.00 mmol) at 0 °C under inert atmosphere. The mixture was stirred at rt for 16 h. The progress of the reaction was monitored by TLC and LCMS. After completion of the reaction, the solvent was evaporated under reduced pressure. A solid was collected. This solid was washed with n-hexane to give 2-allyl-6-((4-fluorophenyl)amino)-1-(2-(piperidin-4-yloxy)pyrimidin-4-yl)-1,2-dihydro-3H-pyrazolo[3,4-d]pyrimidin-3-one (240 mg, 0.498 mmol, 93% yield) as an off-white solid. 40 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to give the pure compound (6.98 mg).

### 2-allyl-6-((4-fluorophenyl)amino)-1-(2-((1-methylpiperidin-4-yl)oxy)pyrimidin-4-yl)-1,2-dihydro-3H-pyrazolo[3,4-d]pyrimidin-3-one

To a stirred solution of 2-allyl-6-((4-fluorophenyl)amino)-1-(2-(piperidin-4-yloxy)pyrimidin-4-yl)-1,2-dihydro-3H-pyrazolo[3,4-d]pyrimidin-3-one (300 mg, 0.649 mmol) in THF (4 ml) was added 37% formaldehyde (0.240 ml, 1.946 mmol) followed by the addition of STAB (687 mg, 3.24 mmol). The mixture was stirred at rt for 1 h. The progress of the reaction was monitored by TLC and LCMS. After completion of the reaction, the reaction mixture was quenched with NH₃ in MeOH followed by TFA at rt and extracted with 10% MeOH in DCM (2×100 mL). The combined organic layers was dried over anhydrous Na₂SO₄. The solvent was evaporated under reduced pressure to give the crude compound which was purified by prep HPLC (X Select C18, 19×250, Mobile phase A: 10 mm Ammonium bicarbonate in water in H₂O, Mobile phase B: acetonitrile) to give 2-allyl-6-((4-fluorophenyl)amino)-1-(2-((1-methylpiperidin-4-yl)oxy)pyrimidin-4-yl)-1,2-dihydro-3H-pyrazolo[3,4-d]pyrimidin-3-one (26 mg, 0.054 mmol, 8.4% yield) as a white solid.

### Example 204. Synthesis of 2-allyl-1-(2-(piperidin-4-yloxy)pyrimidin-4-yl)-6-((4-(2,2,2-trifluoroethoxy)phenyl)amino)-1,2-dihydro-3H-pyrazolo[3,4-d]pyrimidin-3-one (Compound 252) and 2-allyl-1-(2-((1-methylpiperidin-4-yl)oxy)pyrimidin-4-yl)-6-((4-(2,2,2-trifluoroethoxy)phenyl)amino)-1,2-dihydro-3H-pyrazolo[3,4-d]pyrimidin-3-one (Compound 253)

### 4-iodo-2-(methylthio)pyrimidine

To the solution of 4-chloro-2-(methylthio)pyrimidine (1 g, 6.23 mmol) in a vial, was added hydriodic acid (10 ml, 73.1 mmol) at 0 °C and stirred at rt for 16 h. The progress of the reaction was monitored by TLC and LCMS. After completion of the reaction, the solid was filtered, and the collected solid was dissolved in water quenched with aq. NaHCO₃ (2×50 ml) and extracted with EA (2×100 mL). The collected organic layer was washed with aq. sodium thiosulfate (100 mL), and the solvent was evaporated under reduced pressure to get the pure compound 4-iodo-2-(methylthio)pyrimidine (800 mg, 3.01 mmol, 48.4% yield) as a gummy colorless solid.

### 4-iodo-2-(methylsulfonyl)pyrimidine

To a stirred solution of 4-iodo-2-(methylthio)pyrimidine (1 g, 3.97 mmol) in DCM (10 ml) was added mCPBA (1.369 g, 7.93 mmol) at rt under an inert atmosphere. The mixture was stirred at rt for 2 h. The progress of the reaction was monitored by TLC and LCMS. After completion of the reaction, was added aq. NaHCO₃ (50 mL) and the mixture was extracted with EA (2×100 mL). The combined organic layers was dried over anhydrous Na₂SO₄. The solvent was evaporated under reduced pressure to give the crude 4-iodo-2-(methylsulfonyl)pyrimidine (3, 1.1 g, 1.700 mmol, 42.9% yield) as an off-white solid. This compound was taken as such for the next step without any further purification.

### tert-butyl 4-((4-iodopyrimidin-2-yl)oxy)piperidine-1-carboxylate

To a stirred solution of tert-butyl 4-hydroxypiperidine-1-carboxylate (800 mg, 3.97 mmol) in THF (10 ml) at 0 °C under inert atmosphere was added NaH (477 mg, 11.92 mmol), followed by the addition of 4-iodo-2-(methylsulfonyl)pyrimidine (1.129 g, 3.97 mmol), which was dissolved in THF (5 mL). The mixture was stirred at rt for 20 min. The progress of the reaction was monitored by TLC and LCMS. After completion of the reaction, the reaction was quenched with ice-cold water (50 mL) at 0 °C and extracted with EA (2×100 mL). The combined organic layers were dried over anhydrous Na₂SO₄. The solvent was evaporated under reduced pressure to give the crude compound which was purified by column chromatography (SiO₂/230-400 mesh; 20-40% EA-n-hexane) to give tert-butyl 4-((4-iodopyrimidin-2-yl)oxy)piperidine-1-carboxylate (300 mg, 0.600 mmol, 15.09% yield) as an off-white solid.

### tert-butyl 4-((4-(2-allyl-6-(methylthio)-3-oxo-2,3-dihydro-1H-pyrazolo[3,4-d]pyrimidin-1-yl)pyrimidin-2-yl)oxy)piperidine-1-carboxylate

To a stirred solution of 2-allyl-6-(methylthio)-1,2-dihydro-3H-pyrazolo[3,4-d]pyrimidin-3-one (2.5 g, 11.25 mmol) in dioxane (5 ml) was added tert-butyl 4-((4-iodopyrimidin-2-yl)oxy)piperidine-1-carboxylate (4.56 g, 11.25 mmol) followed by the addition of K₂CO₃ (4.66 g, 33.7 mmol) and N,N′-dimethyl ethylene diamine (0.990 g, 11.25 mmol). The mixture was degassed for 30 min, then was added copper (I) iodide (2.142 g, 11.25 mmol) and again the mixture was degassed for 5 min and then stirred at 100 °C for 2 h. The progress of the reaction was monitored by TLC and LCMS. After completion of the reaction, the reaction mass was filtered through celite and washed with EA (2×20 mL). The combined filtrate was dried over anhydrous Na₂SO₄. The solvent was evaporated under reduced pressure to give the crude compound which was purified by column chromatography (SiO₂/230-400 mesh; 55-70% EA-n-hexane) to give tert-butyl 4-((4-(2-allyl-6-(methylthio)-3-oxo-2,3-dihydro-1H-pyrazolo[3,4-d]pyrimidin-1-yl)pyrimidin-2-yl)oxy)piperidine-1-carboxylate (700 mg, 1.149 mmol, 10.21% yield) as an off-white solid.

### tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylthio)-3-oxo-2,3-dihydro-1H-pyrazolo[3,4-d]pyrimidin-1-yl)pyrimidin-2-yl)oxy)piperidine-1-carboxylate (450 mg, 0.901 mmol) in DCM (10 ml) was added mCPBA (311 mg, 1.801 mmol) and the mixture was stirred at rt for 2 h. The progress of the reaction was monitored by TLC and LCMS. After completion of the reaction, aq. NaHCO₃ solution was added and the mixture was extracted with EA (2×100 mL). The combined organic layers was dried over anhydrous Na₂SO₄. The solvents were evaporated under reduced pressure to give the crude compound tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (400 mg, 0.241 mmol, 26.7% yield). This crude compound, as such, is taken for the next step without any purification.

### tert-butyl 4-((4-(2-allyl-3-oxo-6-((4-(2,2,2-trifluoroethoxy)phenyl)amino)-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (400 mg, 0.752 mmol) in acetic acid (2 ml) was added 4-(2,2,2-trifluoroethoxy)aniline (216 mg, 1.129 mmol) at rt under inert atmosphere. The mixture was stirred for 16 h. The progress of the reaction was monitored by TLC and LCMS. After completion of the reaction, the solvent was evaporated under reduced pressure, ice was added and the resulting mixture slowly quenched with aq. NaHCO₃ (10 mL). A solid appeared that was filtered and dried under vacuum. The collected solid was washed with MTBE (4 mL) and n-hexane (10 mL) to give tert-butyl 4-((4-(2-allyl-3-oxo-6-((4-(2,2,2-trifluoroethoxy)phenyl)amino)-2,3-dihydro-1H-pyrazolo[3,4-d]pyrimidin-1-yl)pyrimidin-2-yl)oxy)piperidine-1-carboxylate (400 mg, 0.548 mmol, 72.8% yield) as an off-white solid.

### 2-allyl-1-(2-(piperidin-4-yloxy)pyrimidin-4-yl)-6-((4-(2,2,2-trifluoroethoxy)-phenyl)amino)-1,2-dihydro-3H-pyrazolo[3,4-d]pyrimidin-3-one

To a stirred solution of tert-butyl 4-((4-(2-allyl-3-oxo-6-((4-(2,2,2-trifluoroethoxy)phenyl)amino)-2,3-dihydro-1H-pyrazolo[3,4-d]pyrimidin-1-yl)pyrimidin-2-yl)oxy)piperidine-1-carboxylate (450 mg, 0.700 mmol) in dioxane (3 ml) was added 4M HCl in dioxane (2 mL, 8.00 mmol) at 0 °C under inert atmosphere. The mixture was stirred at rt for 16 h. The progress of the reaction was monitored by TLC and LCMS. After completion of the reaction, the solvent was evaporated under reduced pressure, and it appeared a solid that was collected. This solid was washed with n-hexane to give 2-allyl-1-(2-(piperidin-4-yloxy)pyrimidin-4-yl)-6-((4-(2,2,2-trifluoroethoxy)phenyl)amino)-1,2-dihydro-3H-pyrazolo[3,4-d]pyrimidin-3-one (360 mg, 0.66 mmol, 94.7% yield) as an off-white solid. 60 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to get the pure compound (31.2 mg).

### 2-allyl-1-(2-((1-methylpiperidin-4-yl)oxy)pyrimidin-4-yl)-6-((4-(2,2,2-trifluoroethoxy)phenyl)amino)-1,2-dihydro-3H-pyrazolo[3,4-d]pyrimidin-3-one

To a stirred solution of 2-allyl-1-(2-(piperidin-4-yloxy)pyrimidin-4-yl)-6-((4-(2,2,2-trifluoroethoxy)phenyl)amino)-1,2-dihydro-3H-pyrazolo[3,4-d]pyrimidin-3-one (250 mg, 0.461 mmol) in THF (4 ml) was added formaldehyde (2.88 ml, 2.304 mmol) followed by the addition of STAB (293 mg, 1.382 mmol) and the mixture was stirred at rt for 1 h. The progress of the reaction was monitored by TLC and LCMS. After completion of the reaction, the reaction mixture was quenched with NH₃ in MeOH followed by TFA at rt and extracted with 10% MeOH in DCM (2×100 mL). The combined organic layers was dried over anhydrous Na₂SO₄. The solvent was evaporated under reduced pressure to give the crude compound which was purified by prep HPLC (X Select C18, 19×250, Mobile phase A: 10 mm Ammonium bicarbonate in water in H₂O, Mobile phase B: acetonitrile) to give 2-allyl-1-(2-((1-methylpiperidin-4-yl)oxy)pyrimidin-4-yl)-6-((4-(2,2,2-trifluoroethoxy)phenyl)amino)-1,2-dihydro-3H-pyrazolo[3,4-d]pyrimidin-3-one (43 mg, 0.076 mmol, 16.56% yield) as a white solid.

### Example 205. Synthesis of 2-allyl-6-((4-ethoxyphenyl)amino)-1-(6-(piperidin-4-yloxy)pyridin-2-yl)-1,2-dihydro-3H-pyrazolo[3,4-d]pyrimidin-3-one (Compound 254) and 2-allyl-6-((4-ethoxyphenyl)amino)-1-(6-((1-methylpiperidin-4-yl)oxy)pyridin-2-yl)-1,2-dihydro-3H-pyrazolo[3,4-d]pyrimidin-3-one (Compound 255)

### 1-ethoxy-4-nitrobenzene

To a stirred solution of 4-nitrophenol (1, 1.5 g, 10.78 mmol) in DMF (10 ml) was added bromoethane (2, 0.959 ml, 12.94 mmol) and K₂CO₃ (4.47 g, 32.3 mmol) and the mixture was stirred at 100 °C for 2 h. The progress of the reaction was monitored by TLC and LCMS. After completion of the reaction, the mixture was with water (250 mL) and extracted with ethyl acetate (2×250 mL). The combined organic extracts were washed with brine (100 mL), dried over anhydrous Na₂SO₄, filtered and concentrated under reduced pressure to afford 1-ethoxy-4-nitrobenzene (1.7 g, 10.07 mmol, 93% yield) as an off-white solid.

### 4-ethoxyaniline

To a stirred solution of 1-ethoxy-4-nitrobenzene (1.5 g, 8.97 mmol) in ethanol (20 ml) was added Pd/C (0.955 g, 8.97 mmol) and the mixture was stirred at rt for 16 h under a hydrogen bladder atmosphere. The progress of the reaction was monitored by TLC and LCMS. After completion of the reaction, the reaction mixture was filtered through celite. The collected filtrate was concentrated under reduced pressure to afford crude compound 4-ethoxyaniline (1.2 g, 7.96 mmol, 89% yield) as a brown liquid compound.

### tert-butyl 4-((6-(2-allyl-6-((4-ethoxyphenyl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (1.0 g, 1.885 mmol) in AcOH (10 ml) was added 4-ethoxyaniline (0.310 g, 2.262 mmol) and the mixture was stirred at rt for 16 h. The progress of the reaction was monitored by TLC and LCMS. The reaction mixture was diluted with water (250 mL) and extracted with ethyl acetate (250 mL×2). The combined organic extracts was washed with brine (100 mL), dried over anhydrous Na₂SO₄, filtered and concentrated under reduced pressure to afford crude compound which was purified column chromatography (SiO₂/230-400 mesh; 50-70% ethyl acetate in n-hexane) to give tert-butyl 4-((6-(2-allyl-6-((4-ethoxyphenyl)amino)-3-oxo-2,3-dihydro-1H-pyrazolo[3,4-d]pyrimidin-1-yl)pyridin-2-yl)oxy)piperidine-1-carboxylate (500 mg, 0.817 mmol, 43.3% yield) as an off-white solid.

### 2-allyl-6-((4-ethoxyphenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-ethoxyphenyl)amino)-3-oxo-2,3-dihydro-1H-pyrazolo[3,4-d]pyrimidin-1-yl)pyridin-2-yl)oxy)piperidine-1-carboxylate (500 mg, 0.851 mmol) in 1,4-dioxane (30 ml) was added 4M HCl in 1,4-dioxane (1 mL, 4.25 mmol) and the resulting mixture was stirred at rt for 16 h. The progress of the reaction was monitored by TLC and LCMS. After completion of the reaction, the solvent was evaporated under reduced pressure; the residue obtained was washed with n-hexane, filtered and concentrated under reduced pressure to afford crude 2-allyl-6-((4-ethoxyphenyl)amino)-1-(6-(piperidin-4-yloxy)pyridin-2-yl)-1,2-dihydro-3H-pyrazolo[3,4-d]pyrimidin-3-one (450 mg, 0.785 mmol, 92% yield) as a yellow solid. 50 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to give the pure compound (26.49 mg).

### 2-allyl-6-((4-ethoxyphenyl)amino)-1-(6-((1-methylpiperidin-4-yl)oxy)pyridin-2-yl)-1,2-dihydro-3H-pyrazolo[3,4-d]pyrimidin-3-one

To the stirred solution of 2-allyl-6-((4-ethoxyphenyl)amino)-1-(6-(piperidin-4-yloxy)pyridin-2-yl)-1,2-dihydro-3H-pyrazolo[3,4-d]pyrimidin-3-one (450 mg, 0.923 mmol) in THF (10 ml), was added formaldehyde (0.5 ml, 13.57 mmol). The mixture was stirred at rt for 1 h and then was added STAB (105 mg, 2.77 mmol) and the resulting mixture was allowed to stir for 1 h. The progress of the reaction was monitored by TLC and LCMS. After completion of the reaction, reaction mixture was quenched with TFA, followed by methanolic NH₃ and water. The mixture was extracted with 10% MeOH in DCM (2×100 mL). The combined organic layers was dried over anhydrous Na₂SO₄, filtered and concentrated under reduced pressure to afford crude compound which was purified by prep HPLC to give 2-allyl-6-((4-ethoxyphenyl)amino)-1-(6-((1-methylpiperidin-4-yl)oxy)pyridin-2-yl)-1,2-dihydro-3H-pyrazolo[3,4-d]pyrimidin-3-one (150 mg, 0.293 mmol, 31.8% yield) as an off-white solid.

### Example 206. Synthesis of 2-allyl-6-((4-isobutoxyphenyl)amino)-1-(6-(piperidin-4-yloxy)pyridin-2-yl)-1,2-dihydro-3H-pyrazolo[3,4-d]pyrimidin-3-one (Compound 256) and 2-allyl-6-((4-isobutoxyphenyl)amino)-1-(6-((1-methylpiperidin-4-yl)oxy)pyridin-2-yl)-1,2-dihydro-3H-pyrazolo[3,4-d]pyrimidin-3-one (Compound 257)

### 1-isobutoxy-4-nitrobenzene

To a stirred solution of 4-nitrophenol (1 g, 7.19 mmol) in DMF (10 ml) was added potassium carbonate (2.98 g, 21.57 mmol) and 1-bromo-2-methylpropane (0.782 ml, 7.19 mmol) at room temperature under inert atmosphere. Then, the resulting reaction mixture was stirred at 90 °C for 16 h. The progress of the reaction was monitored by TLC and LCMS. After completion of the reaction, the reaction mixture was quenched with water and extracted with DCM to give crude compound, which was purified by column chromatography (SiO₂/100-200 mesh; 40-50% ethyl acetate-hexane) to give 1-isobutoxy-4-nitrobenzene (700 mg, 2.87 mmol, 39.9% yield) as an off-white solid.

### 4-isobutoxyaniline

To a degassed solution of 1-isobutoxy-4-nitrobenzene (800 mg, 4.10 mmol) in methanol (20 ml) was added 10% Pd—C (436 mg, 4.10 mmol) under nitrogen atmosphere. The reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite to remove the catalyst; the celite pad was washed with methanol (100 mL), and the filtrate was concentrated under reduced pressure to give 4-isobutoxyaniline (700 mg, 3.39 mmol, 83% yield) as a black gummy solid. This compound, as such, is taken for the next step without further purification.

### tert-butyl 4-((6-(2-allyl-6-((4-isobutoxyphenyl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (800 mg, 1.508 mmol) in acetic acid (5 ml) was added 4-isobutoxyaniline (498 mg, 3.02 mmol) at room temperature. The resulting reaction mixture was stirred at 25 °C for 16 h. The progress of the reaction was monitored by TLC and LCMS. After completion of the reaction, the reaction mixture was diluted with water and extracted with 10% MeOH-DCM (2×50 mL). The combined organic layers was dried over Na₂SO₄, filtered, and concentrated under reduced pressure to give crude tert-butyl 4-((6-(2-allyl-6-((4-isobutoxyphenyl)amino)-3-oxo-2,3-dihydro-1H-pyrazolo[3,4-d]pyrimidin-1-yl)pyridin-2-yl)oxy)piperidine-1-carboxylate (800 mg, 1.299 mmol, 86% yield) as a brown gummy solid.

### 2-allyl-6-((4-isobutoxyphenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-isobutoxyphenyl)amino)-3-oxo-2,3-dihydro-1H-pyrazolo[3,4-d]pyrimidin-1-yl)pyridin-2-yl)oxy)piperidine-1-carboxylate (100 mg, 0.162 mmol) in DCM (2 ml) was added 4M HCl in dioxane (3 ml, 12 mmol) at 0 °C and under inert atmosphere. The resulting reaction mixture was stirred at room temperature for 16 h. The progress of the reaction was monitored by TLC and LCMS. After completion of the reaction, the reaction mixture was concentrated under reduced pressure. The crude residue was washed with sodium bicarbonate and extracted with DCM to give crude compound, which was purified by Prep HPLC (X Select C18, 19×250, Mobile phase A: 0.1% FA in H₂O, Mobile phase B: acetonitrile) to give 2-allyl-6-((4-isobutoxyphenyl)amino)-1-(6-(piperidin-4-yloxy)pyridin-2-yl)-1,2-dihydro-3H-pyrazolo[3,4-d]pyrimidin-3-one (10 mg, 0.019 mmol, 11.94% yield) as an off-white solid.

### 2-allyl-6-((4-isobutoxyphenyl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((4-isobutoxyphenyl)amino)-1-(6-(piperidin-4-yloxy)pyridin-2-yl)-1,2-dihydro-3H-pyrazolo[3,4-d]pyrimidin-3-one (200 mg, 0.388 mmol) in THF (5 ml) were added formaldehyde (31.5 mg, 0.388 mmol) and STAB (247 mg, 1.164 mmol) at 0 °C. The resulting reaction mixture was stirred at room temperature for 16 h. The progress of the reaction was monitored by TLC and LCMS. After completion of the reaction, the reaction mixture was concentrated under reduced pressure. The crude residue was washed with water and extracted with 10% MeOH in DCM. The organic layer was dried over anhydrous Na₂SO₄, and evaporated under reduced pressure to give the crude compound. The crude was purified by prep HPLC (X Select C18, 19×250, Mobile phase A: 0.1% FA in H₂O, Mobile phase B: acetonitrile) to give 2-allyl-6-((4-isobutoxyphenyl)amino)-1-(6-((1-methylpiperidin-4-yl)oxy)pyridin-2-yl)-1,2-dihydro-3H-pyrazolo[3,4-d]pyrimidin-3-one (95 mg, 0.179 mmol, 46.2% yield) as an off-white solid.

### Example 207. Synthesis of 2-allyl-6-((4-(cyclopropylmethoxy)phenyl)amino)-1-(6-(piperidin-4-yloxy)pyridin-2-yl)-1,2-dihydro-3H-pyrazolo[3,4-d]pyrimidin-3-one (Compound 259) and 2-allyl-6-((4-(cyclopropylmethoxy)phenyl)amino)-1-(6-((1-methylpiperidin-4-yl)oxy)pyridin-2-yl)-1,2-dihydro-3H-pyrazolo[3,4-d]pyrimidin-3-one (Compound 258)

### 1-(cyclopropylmethoxy)-4-nitrobenzene

To a stirred solution of 4-nitrophenol (1.5 g, 10.78 mmol) in DMF (10 ml) were added K₂CO₃ (4.47 g, 32.3 mmol) and (bromomethyl)cyclopropane (1.046 ml, 10.78 mmol) at 0 °C under inert atmosphere. The mixture was stirred at 90 °C for 16 h. The progress of the reaction was monitored by TLC and LCMS. After completion of the reaction, ice-cold water was added and the mixture stirred for another 10 minutes. The solid was filtered, washed with n-hexane, and dried under vacuum to give 1-(cyclopropylmethoxy)-4-nitrobenzene (1.2 g, 6.21 mmol, 57.6% yield) as a yellow solid.

### 4-(cyclopropylmethoxy)aniline

To a stirred solution of 1-(cyclopropylmethoxy)-4-nitrobenzene (1 g, 5.18 mmol) in ethanol (10 ml) was added 10% Pd/C (0.551 g, 5.18 mmol) at 25 °C under inert atmosphere, and allowed to stir under H₂ bladder pressure at rt for 16 h. The progress of the reaction was monitored by TLC and LCMS. After completion of the reaction, the reaction mixture was filtered through celite and washed with ethyl acetate (2×100 mL). The filtrate was concentrated under reduced pressure to give 4-(cyclopropylmethoxy)aniline (750 mg, 4.60 mmol, 89% yield) as a brown gummy solid.

### tert-butyl 4-((6-(2-allyl-6-((4-(cyclopropylmethoxy)phenyl)amino)-3-oxo-2,3-dihydro-1H-pyrazolo[3,4-d]pyrimidin-1-yl)pyridin-2-yl)oxy)piperidine-1-carboxylate

To a mixture of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (1 g, 1.885 mmol) in AcOH (15 ml) was added 4-(cyclopropylmethoxy)aniline (0.369 g, 2.262 mmol) at rt under nitrogen atmosphere. The reaction mixture was stirred for 16 h. The progress of the reaction was monitored by TLC and LCMS. After completion of the reaction, the reaction mixture was concentrated under reduced pressure, water was added (100 mL). The resulting mixture was extracted with 10% MeOH in DCM (2×100 mL). The combined organic layers was washed with 10% aq. NaHCO₃, dried over anhydrous Na₂SO₄ solvent, and evaporated under reduced pressure to give crude compound which was purified by column chromatography (silica mesh 100-200 using eluent of 70-80% ethyl acetate and hexane) to give tert-butyl 4-((6-(2-allyl-6-((4-(cyclopropylmethoxy)phenyl)amino)-3-oxo-2,3-dihydro-1H-pyrazolo[3,4-d]pyrimidin-1-yl)pyridin-2-yl)oxy)piperidine-1-carboxylate (460 mg, 0.750 mmol, 39.8% yield).

### 2-allyl-6-((4-(cyclopropylmethoxy)phenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-(cyclopropylmethoxy)phenyl)amino)-3-oxo-2,3-dihydro-1H-pyrazolo[3,4-d]pyrimidin-1-yl)pyridin-2-yl)oxy)piperidine-1-carboxylate (450 mg, 0.734 mmol) in 1,4-dioxane (3 ml) was added 4M HCl in dioxane (0.326 ml, 1.304 mmol) at 0 °C under inert atmosphere. The resulting reaction mixture was stirred at room temperature for 16 h. The progress of the reaction was monitored by TLC and LCMS. After completion of the reaction, the solvent was evaporated under reduced pressure and a solid appeared that was collected and washed with n-hexane to give 2-allyl-6-((4-(cyclopropylmethoxy)phenyl)amino)-1-(6-(piperidin-4-yloxy)pyridin-2-yl)-1,2-dihydro-3H-pyrazolo[3,4-d]pyrimidin-3-one (320 mg, 0.623 mmol, 84.8% yield) as off-white solid. 140 mg of the above compound was taken and purified by Prep HPLC (Column: X-select CSH C18, Mobile phase A: Water (with 0.1% AA), Mobile phase B: acetonitrile, Flow rate-15.0 mL/Min, Rt-12.8) to give the pure compound (101.71 mg).

### 2-allyl-6-((4-(cyclopropylmethoxy)phenyl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((4-(cyclopropylmethoxy)phenyl)amino)-1-(6-(piperidin-4-yloxy)pyridin-2-yl)-1,2-dihydro-3H-pyrazolo[3,4-d]pyrimidin-3-one (270 mg, 0.526 mmol) in THF (10 mL) was added 37% formaldehyde (0.3 mL, 2.63 mmol), followed by the addition of STAB (334 mg, 1.577 mmol) at rt. The mixture was stirred for 1 h. The progress of the reaction was monitored by TLC and LCMS. After completion of the reaction, the reaction mixture was quenched with NH₃ in MeOH followed by TFA at rt and water. The mixture was extracted with 10% MeOH in DCM (2×100 mL). The combined organic layers was dried over anhydrous Na₂SO₄. The solvent was evaporated under reduced pressure to give the crude compound that was purified by prep HPLC (Column: X-select CSH C18, Mobile phase A: Water (with 0.1% AA), Mobile phase B: acetonitrile, Flow rate-15.0 mL/Min, Rt-12.8) to give 2-allyl-6-((4-(cyclopropylmethoxy)phenyl)amino)-1-(6-((1-methylpiperidin-4-yl)oxy)pyridin-2-yl)-1,2-dihydro-3H-pyrazolo[3,4-d]pyrimidin-3-one (68 mg, 0.128 mmol, 24.27% yield) as white solid.

### Example 208. Synthesis of 2-allyl-1-[6-(1-methyl-4-piperidyloxy)-2-pyridyl]-6-[p-(1,4-thiazinan-4-yl)phenylamino]-1,2-dihydro-3H-1,2,5,7-tetraazainden-3-one (Compound 261)

This compound was made using a similar method as described in the synthesis of 2-allyl-6-((6-methylpyridin-3-yl)amino)-1-(6-(piperidin-4-yloxy)pyridin-2-yl)-1,2-dihydro-3H-pyrazolo[3,4-d]pyrimidin-3-one using 4-(p-bromophenyl)-1,4-thiazinane (46.9 mg, 182 μmol) and tert-butyl 4-[6-(2-allyl-6-amino-3-oxo-1,2-dihydro-3H-1,2,5,7-tetraazainden-1-yl)-2-pyridyloxy]-1-piperidinecarboxylate (85 mg). Yield 20 mg.

### Example 209. Synthesis of 2-allyl-6-(4-morpholino-3-toluidino)-1-[6-(4-piperidyloxy)-2-pyridyl]-1,2-dihydro-3H-1,2,5,7-tetraazainden-3-one (Compound 262)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using tert-butyl 4-{6-[2-allyl-6-(methylthio)-3-oxo-1,2-dihydro-3H-1,2,5,7-tetraazainden-1-yl]-2-pyridyloxy}-1-piperidinecarboxylate (0.20 g) and 4-morpholino-3-tolylamine (92.5 mg). Yield 180 mg.

### Example 210. Synthesis of rel-(S)-2-allyl-1-(6-(azepan-4-yloxy)pyridin-2-yl)-6-((4-fluorophenyl)amino)-1,2-dihydro-3H-pyrazolo[3,4-d]pyrimidin-3-one (Compound 266), rel-(S)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one (Compound 351), rel-(R)-2-allyl-1-(6-(azepan-4-yloxy)pyridin-2-yl)-6-((4-fluorophenyl)amino)-1,2-dihydro-3H-pyrazolo[3,4-d]pyrimidin-3-one (Compound 344), and rel-(R)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one (Compound 372)

### tert-butyl4-((6-bromopyridin-2-yl)oxy)azepane-1-carboxylate

To a stirred solution of 2-bromo-6-fluoropyridine (1.5 g, 8.52 mmol) in THF (30 ml) was added NaH (0.686 g, 17.90 mmol) followed by the addition of tert-butyl 4-hydroxyazepane-1-carboxylate (1.835 g, 8.52 mmol) at 0 °C under inert atmosphere and the mixture was allowed to stir at 60 °C for 2 h. The progress of the reaction was monitored by TLC and UPLC. After completion of the reaction, the reaction mixture was quenched with ice-cold water (50 mL) and extracted with ethyl acetate (2×100 mL), dried over anhydrous sodium sulfate, filtered, and concentrated under reduced pressure to give the crude compound, which was purified by column chromatography (silica mesh 100-200 at eluent of 40-50% ethyl acetate and hexane) to give tert-butyl 4-((6-bromopyridin-2-yl)oxy)azepane-1-carboxylate (2.0 g, 5.33 mmol, 62.6% yield) as an off-white solid.

### tert-butyl 4-((6-(2-allyl-6-(methylthio)-3-oxo-2,3-dihydro-1H-pyrazolo[3,4-d]pyrimidin-1-yl)pyridin-2-yl)oxy)azepane-1-carboxylate

To a stirred solution of tert-butyl 4-((6-bromopyridin-2-yl)oxy)azepane-1-carboxylate (5.01 g, 13.50 mmol) in 1,4-dioxane (15 ml) was added 2-allyl-6-(methylthio)-1,2-dihydro-3H-pyrazolo[3,4-d]pyrimidin-3-one (2.5 g, 11.25 mmol), N,N-Dimethylethylenediamine (0.992 g, 11.25 mmol) and K₂CO₃ (4.66 g, 33.7 mmol). The mixture was degassed for 20 min at rt under inert atmosphere, then was added CuI (2.137 g, 11.25 mmol) and again the mixture was degassed for 5 min. The mixture was stirred at 100 °C for 5 h. The progress of the reaction was monitored by TLC and LCMS. After completion of the reaction, the reaction mixture was filtered through celite and the filtrate concentrated under reduced pressure to afford crude compound, which was purified by column chromatography (silica mesh 100-200 at eluent of 40-50% ethyl acetate and hexane) to afford tert-butyl 4-((6-(2-allyl-6-(methylthio)-3-oxo-2,3-dihydro-1H-pyrazolo[3,4-d]pyrimidin-1-yl)pyridin-2-yl)oxy)azepane-1-carboxylate (1.9 g, 3.08 mmol, 27.4% yield) as a gummy solid.

### tert-butyl 4-((6-(2-allyl-6-(methylsulfonyl)-3-oxo-2,3-dihydro-1H-pyrazolo[3,4-d]pyrimidin-1-yl)pyridin-2-yl)oxy)azepane-1-carboxylate

To a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)azepane-1-carboxylate (2.0 g, 3.90 mmol), in DCM (20 ml) was added mCPBA (1.885 g, 8.19 mmol) at 0 °C under inert atmosphere, and the resulting mixture was allowed to stir at rt for 3 h. The progress of the reaction was monitored by TLC and UPLC. After completion of the reaction, the reaction mixture was quenched with aq. NaHCO₃ (100 mL) and extracted with DCM (2×200 mL). The combined organic layers was washed with brine (100 mL), dried over anhydrous sodium sulfate, filtered and concentrated under reduced pressure to give the crude compound tert-butyl 4-((6-(2-allyl-6-(methylsulfonyl)-3-oxo-2,3-dihydro-1H-pyrazolo[3,4-d]pyrimidin-1-yl)pyridin-2-yl)oxy)azepane-1-carboxylate (1.8 g, 1.124 mmol, 28.8% yield). This crude compound was taken for the next step without any further purification.

### tert-butyl 4-((6-(2-allyl-6-((4-fluorophenyl)amino)-3-oxo-2,3-dihydro-1H-pyrazolo[3,4-d]pyrimidin-1-yl)pyridin-2-yl)oxy)azepane-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)azepane-1-carboxylate (1.7 g, 3.12 mmol) in acetic acid (15 ml) was added 4-fluoro aniline (0.296 ml, 3.12 mmol) and the resulting mixture was allowed to stir at rt for 16 h. The progress of the reaction was monitored by TLC and UPLC. After completion of the reaction, the solvent was evaporated under reduced pressure and the residue quenched with aq. NaHCO₃ (100 mL) and extracted with 10% MeOH in DCM (2×200 mL). The combined organic extracts was washed with brine (100 mL), dried over anhydrous sodium sulfate, filtered, and concentrated under reduced pressure to afford crude compound which was purified by column chromatography (silica mesh 100-200 at eluent of 40-50% ethyl acetate and hexane) to give tert-butyl 4-((6-(2-allyl-6-((4-fluorophenyl)amino)-3-oxo-2,3-dihydro-1H-pyrazolo[3,4-d]pyrimidin-1-yl)pyridin-2-yl)oxy)azepane-1-carboxylate (1.0 g, 1.650 mmol, 52.9% yield) as an off-white solid. This pure compound was separated by chiral SFC (I-CELLULOSE-Z, 0.5% IPAm in ACN-IPA-60-40, Oven Temperature: 40, Column Position: 4, BPR Pressure: 102.0 kg/cm²) to get the two enantiomers.

### rel-(R)-2-allyl-1-(6-(azepan-4-yloxy)pyridin-2-yl)-6-((4-fluorophenyl)amino)-1,2-dihydro-3H-pyrazolo[3,4-d]pyrimidin-3-one

To a stirred solution of rel-tert-butyl (R)-4-((6-(2-allyl-6-((4-fluorophenyl)amino)-3-oxo-2,3-dihydro-1H-pyrazolo[3,4-d]pyrimidin-1-yl)pyridin-2-yl)oxy)azepane-1-carboxylate (the first eluting enantiomer, 230 mg, 0.4 mmol) in DCM (5 ml) was added 4M HCl in dioxane (0.1 ml, 0.052 mmol) at 0 °C under inert atmosphere. The mixture was stirred at rt for 16 h. The progress of the reaction was monitored by TLC and LCMS. After completion of the reaction, the solvent was evaporated under reduced pressure to give rel-(R)-2-allyl-1-(6-(azepan-4-yloxy)pyridin-2-yl)-6-((4-fluorophenyl)amino)-1,2-dihydro-3H-pyrazolo[3,4-d]pyrimidin-3-one (180 mg, 0.40 mmol, 94% yield) as an off-white solid. 40 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to get the pure compound (20 mg). Chiral HPLC: 97.381%, tR=4.72 min.

### rel-(R)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one

To a stirred solution of rel-(R)-2-allyl-1-(6-(azepan-4-yloxy)pyridin-2-yl)-6-((4-fluorophenyl)amino)-1,2-dihydro-3H-pyrazolo[3,4-d]pyrimidin-3-one (55 mg, 0.116 mmol) in THF (4 ml) was added 37% formaldehyde (0.1 ml, 0.578 mmol) followed by the addition of STAB (73.5 mg, 0.347 mmol) and the resulting mixture was stirred at rt for 1 h. Progress of the reaction was monitored by TLC and LCMS. After completion of the reaction, the reaction mixture was quenched with NH₃ in MeOH, followed by TFA at rt, and extracted with 10% MeOH in DCM (2×50 mL). The combined organic layers were dried over anhydrous Na₂SO₄. The solvent was evaporated under reduced pressure to give the crude compound, which was purified by prep HPLC (X Select C18, 19×250, Mobile phase A: 10 mm Ammonium bicarbonate in water in H₂O, Mobile phase B: acetonitrile) to give rel-(R)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one (19.52 mg, 0.040 mmol, 34.5% yield) as an off-white solid. Chiral HPLC: 99.145%, tR=4.97 min.

### rel-(S)-2-allyl-1-(6-(azepan-4-yloxy)pyridin-2-yl)-6-((4-fluorophenyl)amino)-1,2-dihydro-3H-pyrazolo[3,4-d]pyrimidin-3-one

To a stirred solution of rel-tert-butyl(S)-4-((6-(2-allyl-6-((4-fluorophenyl)amino)-3-oxo-2,3-dihydro-1H-pyrazolo[3,4-d]pyrimidin-1-yl)pyridin-2-yl)oxy)azepane-1-carboxylate (the second eluting enantiomer, 30 mg, 0.052 mmol) in DCM (2 ml) was added 4M HCl in 1,4-dioxane (0.1 ml, 0.052 mmol) at 0 °C under inert atmosphere and the resulting mixture was stirred for 16 h. The progress of the reaction was monitored by TLC and LCMS. After completion of the reaction, the solvent was evaporated under reduced pressure and washed with n-hexane to give the compound rel-(S)-2-allyl-1-(6-(azepan-4-yloxy)pyridin-2-yl)-6-((4-fluorophenyl)amino)-1,2-dihydro-3H-pyrazolo[3,4-d]pyrimidin-3-one (17 mg, 0.035 mmol, 67.9% yield) as an off-white solid. Chiral HPLC: 97.381%, tR=4.72 min.

### rel-(S)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one

To a stirred solution of rel-(S)-2-allyl-1-(6-(azepan-4-yloxy)pyridin-2-yl)-6-((4-fluorophenyl)amino)-1,2-dihydro-3H-pyrazolo[3,4-d]pyrimidin-3-one (300 mg, 0.631 mmol) in THF (4 ml) was added 37% formaldehyde (1 ml, 0.631 mmol) followed by the addition of STAB (401 mg, 1.893 mmol) and the resulting mixture was stirred at rt for 1 h. The progress of the reaction was monitored by TLC and LCMS. After completion of the reaction, the reaction mixture was quenched with NH₃ in MeOH followed by TFA at rt and extracted with 10% MeOH in DCM (2×100 mL). The combined organic layers were dried over anhydrous Na₂SO₄. The solvent was evaporated under reduced pressure to give the crude compound, which was purified by prep HPLC (X Select C18, 19×250, Mobile phase A: 10 mm Ammonium bicarbonate in water in H₂O, Mobile phase B: acetonitrile) to give rel-(S)-2-allyl-6-((4-fluorophenyl)amino)-1-(6-((1-methylazepan-4-yl)oxy)pyridin-2-yl)-1,2-dihydro-3H-pyrazolo[3,4-d]pyrimidin-3-one (19.52 mg, 0.040 mmol, 34.5% yield) as an off-white solid. Chiral SFC purity: 96.326%, tR=5.43 min.

### Example 211. Synthesis of 2-allyl-1-[6-(1-methyl-4-piperidyloxy)-2-pyridyl]-6-(4-morpholino-3-toluidino)-1,2-dihydro-3H-1,2,5,7-tetraazainden-3-one (Compound 270)

This compound was made using a similar method as described in the synthesis of 2-allyl-6-((4-chlorophenyl)amino)-1-(6-((1-methylpiperidin-4-yl)oxy)pyridin-2-yl)-1,2-dihydro-3H-pyrazolo[3,4-d]pyrimidin-3-one using 2-allyl-6-(4-morpholino-3-toluidino)-1-[6-(4-piperidyloxy)-2-pyridyl]-1,2-dihydro-3H-1,2,5,7-tetraazainden-3-one.

### Example 212. Synthesis of 2-allyl-6-((2-methyl-2H-indazol-5-yl)amino)-1-(6-(piperidin-4-yloxy)pyridin-2-yl)-1,2-dihydro-3H-pyrazolo[3,4-d]pyrimidin-3-one (Compound 274) and 2-allyl-6-((2-methyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 273)

### tert-butyl 4-((6-(2-allyl-6-((2-methyl-2H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (950 mg, 1.790 mmol) in acetonitrile (5 mL) was added 2-methyl-2H-indazol-5-amine (290 mg, 1.969 mmol) at room temperature. The resultant reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude was purified by flash column chromatography (silica gel, 100-200 mesh size) using methanol-DCM (15-20%) as an eluent to obtain tert-butyl 4-((6-(2-allyl-6-((2-methyl-2H-indazol-5-yl)amino)-3-oxo-2,3-dihydro-1H-pyrazolo[3,4-d]pyrimidin-1-yl)pyridin-2-yl)oxy)piperidine-1-carboxylate (550 mg, 0.782 mmol, 43.7% yield) as a brown solid.

### 2-allyl-6-((2-methyl-2H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((2-methyl-2H-indazol-5-yl)amino)-3-oxo-2,3-dihydro-1H-pyrazolo[3,4-d]pyrimidin-1-yl)pyridin-2-yl)oxy)piperidine-1-carboxylate (550 mg, 0.920 mmol) in 1,4-dioxane (5 mL), was added 4M HCl in 1,4-dioxane (2.0 mL) at 0 °C. The reaction mixture was stirred at room temperature for 4 h. The progress of the reaction was monitored by TLC. After completion of the reaction, the reaction mixture was concentrated under reduced pressure. The resultant crude residue was purified by prep. HPLC X-Select C18 (19*250, 5 mL), Mobile phase A: 0.1 M FA in H₂O, Mobile phase B: acetonitrile) to give 2-allyl-6-((2-methyl-2H-indazol-5-yl)amino)-1-(6-(piperidin-4-yloxy)pyridin-2-yl)-1,2-dihydro-3H-pyrazolo[3,4-d]pyrimidin-3-one (55 mg, 0.109 mmol, 11.89% yield) as an off white-solid.

### 2-allyl-6-((2-methyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((2-methyl-2H-indazol-5-yl)amino)-1-(6-(piperidin-4-yloxy)pyridin-2-yl)-1,2-dihydro-3H-pyrazolo[3,4-d]pyrimidin-3-one (450 mg, 0.904 mmol) in THF (10 mL) was added 37% formaldehyde (5 mL) at 25 °C. The reaction mixture was stirred at 25 °C for 5 min. STAB (575 mg, 2.71 mmol) was added portion-wise. After the complete addition of STAB, the reaction mixture was stirred at 25 °C for 20 min. The progress of the reaction was monitored by TLC and LCMS. The reaction mixture was quenched with TFA, followed by methanolic ammonia 2 molar solution diluted with water and extracted with 10% MeOH in DCM (2×100 mL). The combined organic extracts was washed with NaHCO₃, dried over anhydrous Na₂SO₄, filtered, and concentrated under reduced pressure to afford crude compound, which was purified by prep. HPLC ZORBAX (30 MM), Mobile phase A: 10 MM ABC in H₂O, Mobile phase B: acetonitrile) to give 2-allyl-6-((2-methyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one (185 mg, 0.347 mmol, 38.4% yield) as an off-white solid.

### Example 213. Synthesis of 2-allyl-6-((3-chloro-1H-indazol-5-yl)amino)-1-(6-(piperidin-4-yloxy)pyridin-2-yl)-1,2-dihydro-3H-pyrazolo[3,4-d]pyrimidin-3-one (Compound 275)

### tert-butyl 4-((6-(2-allyl-6-((3-chloro-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (600 mg, 1.166 mmol) in acetonitrile (5 mL), 3-chloro-1H-indazol-5-amine (215 mg, 1.283 mmol) was added at room temperature. The resulting reaction mixture was subjected to stirring at 75 °C for 16 h. The progress of reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The above crude was purified by flash column chromatography (silica gel, 100-200 mesh size) using ethylacetate-hexane (40% to 50%) as an eluent to obtain tert-butyl 4-((6-(2-allyl-6-((3-chloro-1H-indazol-5-yl)amino)-3-oxo-2,3-dihydro-1H-pyrazolo[3,4-d]pyrimidin-1-yl)pyridin-2-yl)oxy)piperidine-1-carboxylate (400 mg, 0.563 mmol, 48.3% yield) as a brown solid.

### 2-allyl-6-((3-chloro-1H-indazol-5-yl)amino)-1-(6-(piperidin-4-yloxy)pyridin-2-yl)-1,2-dihydro-3H-pyrazolo[3,4-d]pyrimidin-3-one

To a stirred solution tert-butyl 4-((6-(2-allyl-6-((3-chloro-1H-indazol-5-yl)amino)-3-oxo-2,3-dihydro-1H-pyrazolo[3,4-d]pyrimidin-1-yl)pyridin-2-yl)oxy)piperidine-1-carboxylate (400 mg, 0.647 mmol) in 1,4-dioxane (5 mL), was added HCl in 1,4-dioxane (4M, 2.0 mL) at 0 °C. The resulting reaction mixture was stirred at room temperature for 4 h. The progress of the reaction was monitored by TLC. After completion of the reaction, the reaction mixture was concentrated under reduced pressure. The resultant crude residue was purified by prep. HPLC X-Select C18 (19*250, 5 mL), Mobile phase A: 0.1 FA in H₂O, Mobile phase B: acetonitrile) to give 2-allyl-6-((3-chloro-1H-indazol-5-yl)amino)-1-(6-(piperidin-4-yloxy)pyridin-2-yl)-1,2-dihydro-3H-pyrazolo[3,4-d]pyrimidin-3-one (92 mg, 0.177 mmol, 68% yield) as an off white-solid.

### Example 214. Synthesis of Tert-butyl 4-((6-(2-allyl-6-((2-ethyl-2H-indazol-5-yl)amino)-3-oxo-2,3-dihydro-1H-pyrazolo[3,4-d]pyrimidin-1-yl)pyridin-2-yl)oxy)piperidine-1-carboxylate (Compound 276) and 2-allyl-6-((2-ethyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 282)

### tert-butyl 4-((6-(2-allyl-6-((2-ethyl-2H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (1 g, 1.943 mmol) in acetonitrile (15 mL) was added 2-ethyl-2H-indazol-5-amine (0.470 g, 2.91 mmol) at room temperature. The reaction mixture was stirred at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The above crude was purified by flash column chromatography (silica gel, 100-200 mesh size) using ethylacetate-hexane (40% to 50%) as an eluent to obtain the desired product tert-butyl 4-((6-(2-allyl-6-((2-ethyl-2H-indazol-5-yl)amino)-3-oxo-2,3-dihydro-1H-pyrazolo[3,4-d]pyrimidin-1-yl)pyridin-2-yl)oxy)piperidine-1-carboxylate (1.2 g, 1.373 mmol, 70.7% yield) as a brown solid.

### 2-allyl-6-((2-ethyl-2H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((2-ethyl-2H-indazol-5-yl)amino)-3-oxo-2,3-dihydro-1H-pyrazolo[3,4-d]pyrimidin-1-yl)pyridin-2-yl)oxy)piperidine-1-carboxylate (150 mg, 0.172 in 1,4-dioxane (5 mL), was added 4M HCl in 1,4-dioxane (2.0 mL) at 0 °C. The resulting reaction mixture was stirred at room temperature for 4 h. The reaction was monitored by TLC. After completion of the reaction, the reaction mixture was concentrated under reduced pressure. The resultant crude residue was purified by prep. HPLC (Shimpack 150*19 5 um, Mobile phase A: 10 MM ABC in H₂O, Mobile phase B: acetonitrile) to give 2-allyl-6-((2-ethyl-2H-indazol-5-yl)amino)-1-(6-(piperidin-4-yloxy)pyridin-2-yl)-1,2-dihydro-3H-pyrazolo[3,4-d]pyrimidin-3-one (43.4 mg, 0.083 mmol, 48.4% yield) as an off white-solid.

### 2-allyl-6-((2-ethyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((2-ethyl-2H-indazol-5-yl)amino)-1-(6-(piperidin-4-yloxy)pyridin-2-yl)-1,2-dihydro-3H-pyrazolo[3,4-d]pyrimidin-3-one (1 g, 1.955 mmol) in THF (10 mL) was added formaldehyde (0.437 ml, 5.86 mmol) at 25 °C. The reaction mixture was stirred at 25 °C for 5 min. Then, STAB (0.829 g, 3.91 mmol) was added portion-wise. After the complete addition of STAB, the reaction mixture was stirred at 25 °C for 20 minutes. The progress of the reaction was monitored by TLC and LCMS. The reaction mixture was quenched with TFA followed by NH₃ in MeOH, diluted with water and extracted with 10% MeOH in DCM (2×100 mL). The above combined organic extracts were washed with NaHCO₃, dried over anhydrous Na₂SO₄, filtered, and concentrated under reduced pressure. The above crude was purified by prep. HPLC (X-select C18,250 mm×19, Mobile phase A: 0.1% FA in H₂O, Mobile phase B: acetonitrile), to afford 2-allyl-6-((2-ethyl-2H-indazol-5-yl)amino)-1-(6-((1-methylpiperidin-4-yl)oxy)pyridin-2-yl)-1,2-dihydro-3H-pyrazolo[3,4-d]pyrimidin-3-one (162 mg, 0.302 mmol, 15.45% yield) as an off-white solid.

### Example 215. Synthesis of 2-allyl-1-(6-((1-methylpiperidin-4-yl)oxy)pyridin-2-yl)-6-((4-morpholinophenyl)amino)-1,2-dihydro-3H-pyrazolo[3,4-d]pyrimidin-3-one (Compound 278)

### tert-butyl 4-((6-(2-allyl-6-((4-morpholinophenyl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (500 mg, 0.972 mmol) in acetonitrile (10 mL) was added 4-morpholinoaniline (346 mg, 1.943 mmol) at room temperature. The reaction mixture was stirred at 70 °C for 16 h. The progress of reaction was monitored by LCMS, After the completion of the reaction, the reaction mixture was concentrated under reduced pressure to obtain crude product. The crude product was purified by flash column chromatography (silica gel, 100-200) using ethyl acetate-hexane (50 to 100%) as an eluent to obtain tert-butyl 4-((6-(2-allyl-6-((4-morpholinophenyl)amino)-3-oxo-2,3-dihydro-1H-pyrazolo[3,4-d]pyrimidin-1-yl)pyridin-2-yl)oxy)piperidine-1-carboxylate (500 mg, 0.445 mmol, 45.8% yield) as a red colored gum liquid.

### 2-allyl-6-((4-morpholinophenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-morpholinophenyl)amino)-3-oxo-2,3-dihydro-1H-pyrazolo[3,4-d]pyrimidin-1-yl)pyridin-2-yl)oxy)piperidine-1-carboxylate (500 mg, 0.445 mmol) in 1,4-dioxane (10 mL), was added 4 N HCl in 1,4-dioxane (5.0 mL) at 0 °C. The resulting reaction mixture was stirred at room temperature for 4 h. The progress of reaction was monitored by TLC. After completion of the reaction, the reaction mixture was concentrated under reduced pressure. The resultant crude residue was purified by prep-HPLC X-SELECT C18 (19*150 mm) 5u, Mobile phase A: 0.1% FA in Water, Mobile phase B: acetonitrile) to give 2-allyl-6-((4-morpholinophenyl)amino)-1-(6-(piperidin-4-yloxy)pyridin-2-yl)-1,2-dihydro-3H-pyrazolo[3,4-d]pyrimidin-3-one (21.9 mg, 0.041 mmol, 9.22% yield) as a white fluffy solid.

### 2-allyl-1-(6-((1-methylpiperidin-4-yl)oxy)pyridin-2-yl)-6-((4-morpholinophenyl)amino)-1,2-dihydro-3H-pyrazolo[3,4-d]pyrimidin-3-one

To a stirred solution of 2-allyl-6-((4-morpholinophenyl)amino)-1-(6-(piperidin-4-yloxy)pyridin-2-yl)-1,2-dihydro-3H-pyrazolo[3,4-d]pyrimidin-3-one (1 g, 1.892 mmol) in THF (20 mL) was added 37% aq. formaldehyde (5 mL) at 25 °C. The reaction mixture stirred at 25 °C for 5 min. sodium triacetoxyborohydride (1.203 g, 5.68 mmol) was added portion-wise. After the complete addition of STAB, the reaction mixture was stirred at 25 °C for 1 h. The progress of the reaction was monitored by TLC and LCMS. Then reaction mixture was quenched with TFA followed by NH₃ in MeOH then diluted with water and extracted with 10% MeOH in DCM (2×100 mL). The combined organic extract was washed with NaHCO₃, dried over anhydrous Na₂SO₄, filtered, and concentrated under reduced pressure to afford crude compound which was purified by prep. HPLC (X-SELECT C18 150M, Mobile phase A: 10 MM ABC in H₂O, Mobile phase B: acetonitrile) to give 2-allyl-1-(6-((1-methylpiperidin-4-yl)oxy)pyridin-2-yl)-6-((4-morpholinophenyl)amino)-1,2-dihydro-3H-pyrazolo[3,4-d]pyrimidin-3-one (95 mg, 0.173 mmol, 17.42% yield) as a white fluffy solid.

### Example 216. Synthesis of 2-allyl-6-((1-methyl-1H-indazol-5-yl)amino)-1-(2-(piperidin-4-yloxy)pyrimidin-4-yl)-1,2-dihydro-3H-pyrazolo[3,4-d]pyrimidin-3-one (Compound 350) and 2-allyl-6-((1-methyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one (Compound 279)

### tert-butyl 4-((4-(2-allyl-6-((1-methyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (0.7 g, 1.317 mmol) in acetic acid (10 ml) was added 1-methyl-1H-indazol-5-amine (2, 0.194 g, 1.317 mmol) at 0 °C. The resulting reaction mixture was stirred at room temperature for 16 h. The progress of reaction was monitored by TLC. After completion of the reaction, the reaction mixture was concentrated under reduced pressure. The resultant crude residue was washed with sodium bicarbonate and extracted with DCM to give crude compound. The crude compound was purified by column chromatography to give tert-butyl 4-((4-(2-allyl-6-((1-methyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate (300 mg, 0.436 mmol, 33.1% yield) as off white solid.

### 2-allyl-6-((1-methyl-1H-indazol-5-yl)amino)-1-(2-(piperidin-4-yloxy)pyrimidin-4-yl)-1,2-dihydro-3H-pyrazolo[3,4-d]pyrimidin-3-one

To a stirred solution of tert-butyl 4-((4-(2-allyl-6-((1-methyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate (0.270 g, 0.451 mmol) in DCM (10 ml) was added 4 N HCl in 1,4-dioxane (0.1 ml, 0.451 mmol) at 0 °C. The resulting reaction mixture was stirred at room temperature for 16 h. The progress of reaction was monitored by LCMS. After completion of the reaction, the reaction mixture was concentrated under reduced pressure. The resultant crude residue was washed with sodium bicarbonate and extracted with DCM. The solvent was removed in vacuo. The crude compound was submitted to prep. HPLC (Column: X-select CSH C18, Mobile phase A: Water (with 0.1% AA), Mobile phase B: acetonitrile, Flow rate-15.0 mL/Min, Rt-12.8) to give 2-allyl-6-((1-methyl-1H-indazol-5-yl)amino)-1-(2-(piperidin-4-yloxy)pyrimidin-4-yl)-1,2-dihydro-3H-pyrazolo[3,4-d]pyrimidin-3-one (11 mg, 0.022 mmol, 54.9% yield) as off white solid.

### 2-allyl-6-((1-methyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one

To a stirred solution of 2-allyl-6-((1-methyl-1H-indazol-5-yl)amino)-1-(2-(piperidin-4-yloxy)pyrimidin-4-yl)-1,2-dihydro-3H-pyrazolo[3,4-d]pyrimidin-3-one (350 mg, 0.702 mmol) in THF (2 ml) was added 37% formaldehyde (0.1 ml, 0.702 mmol) and the resulting mixture was stirred for 10 min and then was added sodium triacetoxyborohydride (149 mg, 0.702 mmol) at 0 °C. The resulting reaction mixture was stirred at room temperature for 16 h. The progress of reaction was monitored by TLC. The mixture was concentrated under reduced pressure. To the crude residue was added water and the mixture was extracted with DCM. Removal of solvent under reduced pressure gave a crude compound. The crude product was purified by prep HPLC (Shimpack, C18 (20*250 mm), 5 micron) Mobile phase A: 10 mm Ammonium bicarbonate in water, Mobile phase B: acetonitrile) to give 2-allyl-6-((1-methyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one (80 mg, 0.155 mmol, 22.10% yield) as off white solid.

### Example 217. Synthesis of 2-allyl-6-((1-ethyl-1H-indazol-5-yl)amino)-1-(6-(piperidin-4-yloxy)pyridin-2-yl)-1,2-dihydro-3H-pyrazolo[3,4-d]pyrimidin-3-one (Compound 280)

### 1-ethyl-5-nitro-1H-indazole

To a stirred solution of 5-nitro-1H-indazole (1.5 g, 9.19 mmol) in N,N-Dimethylformamide (15 ml) was added sodium hydride (60% in oil, 1.57 g, 10.11 mmol) at 25 °C and the mixture was stirred for 30 min. Then, ethyl iodide (0.817 ml, 10.11 mmol) was added to the reaction mixture under an argon atmosphere. The resulting reaction mixture was stirred at 25 °C for 16 h. The reaction was monitored by TLC. After completion of the reaction, the reaction mixture was poured into ice-cold water, and the precipitated solid was filtered and dried. The resulting crude material was purified by flash chromatography (SiO₂/230-400 mesh; ~20-30% EtOAc/hexane) to afford 1-ethyl-5-nitro-1H-indazole (1.1 g, 5.71 mmol, 62.1% yield) as an off white solid.

### 1-ethyl-1H-indazol-5-amine

To a degassed solution of 1-ethyl-5-nitro-1H-indazole (1.1 g, 5.75 mmol) in EtOH (20 ml) was added 10% Pd/C (400 mg) under an inert atmosphere. Then, the reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure to get 1-ethyl-1H-indazol-5-amine (3, 0.91 g, 4.97 mmol, 86% yield) as a brown gummy solid, which was taken into the next step without further purification.

### tert-butyl 4-((6-(2-allyl-6-((1-ethyl-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (300 mg, 0.583 mmol) in acetonitrile (5 ml) was added 1-ethyl-1H-indazol-5-amine (122 mg, 0.758 mmol) at room temperature. The resulting reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using EtOH-hexane (60-80%) as an eluent to obtain tert-butyl 4-((6-(2-allyl-6-((1-ethyl-1H-indazol-5-yl)amino)-3-oxo-2,3-dihydro-1H-pyrazolo[3,4-d]pyrimidin-1-yl)pyridin-2-yl)oxy)piperidine-1-carboxylate (350 mg, 0.244 mmol, 41.9% yield) as a brown solid.

### 2-allyl-6-((1-ethyl-1H-indazol-5-yl)amino)-1-(6-(piperidin-4-yloxy)pyridin-2-yl)-1,2-dihydro-3H-pyrazolo[3,4-d]pyrimidin-3-one

To a stirred solution tert-butyl 4-((6-(2-allyl-6-((1-ethyl-1H-indazol-5-yl)amino)-3-oxo-2,3-dihydro-1H-pyrazolo[3,4-d]pyrimidin-1-yl)pyridin-2-yl)oxy)piperidine-1-carboxylate (350 mg, 0.572 mmol) in DCM (10 ml), was added TFA (0.220 ml, 2.86 mmol) at 0 °C. The resulting reaction mixture was stirred at room temperature for 12 h. The progress of the reaction was monitored by TLC. After completion of the reaction, the reaction mixture was concentrated under reduced pressure. The crude residue was purified by prep. HPLC X-Select C18 (19*250, 5 mL), Mobile phase A: 0.1 M FA in H2O, Mobile phase B: acetonitrile) to give 2-allyl-6-((1-ethyl-1H-indazol-5-yl)amino)-1-(6-(piperidin-4-yloxy)pyridin-2-yl)-1,2-dihydro-3H-pyrazolo[3,4-d]pyrimidin-3-one (49 mg, 0.095 mmol, 16.55% yield) as an off white-solid.

### Example 218. Synthesis of 2-allyl-6-((1-methyl-1H-indazol-5-yl)amino)-1-(6-((1-(methyl-d3)piperidin-4-yl)oxy)pyridin-2-yl)-1,2-dihydro-3H-pyrazolo[3,4-d]pyrimidin-3-one (Compound 281)

To a stirred solution 2-allyl-6-((1-methyl-1H-indazol-5-yl)amino)-1-(6-(piperidin-4-yloxy)pyridin-2-yl)-1,2-dihydro-3H-pyrazolo[3,4-d]pyrimidin-3-one (400 mg, 0.804 mmol) in THF (10 mL) was added formaldehyde D₂O (20%, 2 ml, 0.804 mmol) at 25 °C. The reaction mixture was stirred at 25 °C for 5 min. Then, sodium borodeuteride (33.7 mg, 0.804 mmol) was added portion-wise, and the reaction mixture was stirred at 25 °C for 20 min. The progress of the reaction was monitored by TLC and LCMS. After completion of the reaction, the reaction mixture was quenched with sodium hydroxide solution (20 mL) diluted with water and extracted with 10% MeOH in DCM (2×100 mL). The combined organic extracts were washed with NaHCO₃, dried over anhydrous Na₂SO₄, filtered, and concentrated under reduced pressure. The residue was purified by prep. HPLC (X-select C18,250 mm×19, Mobile phase A: 10 MM AA in H₂O, Mobile phase B: acetonitrile), factions were lyophilized to give 2-allyl-6-((1-methyl-1H-indazol-5-yl)amino)-1-(6-((1-(methyl-d3)piperidin-4-yl)oxy)pyridin-2-yl)-1,2-dihydro-3H-pyrazolo[3,4-d]pyrimidin-3-one (62 mg, 0.120 mmol, 14.94% yield) as an off-white solid.

### Example 219. Synthesis of 2-ethyl-6-[(1-methyl-1H-indazol-5-yl)amino]-1-{6-[(piperidin-4-yl)amino]pyridin-2-yl}-1H,2H,3H-pyrazolo[3,4-d]pyrimidin-3-one (Compound 285)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using tert-butyl 4-({6-[2-ethyl-6-(methylsulfanyl)-3-oxo-1H,2H,3H-pyrazolo[3,4-d]pyrimidin-1-yl]pyridin-2-yl}amino)piperidine-1-carboxylate (190 mg) and 1-methyl-1H-indazol-5-amine (1 equiv.). Yield: 136 mg TFA salt (58%) as a powder.

### Example 220. Synthesis of 2-ethyl-6-[(1-methyl-1H-indazol-5-yl)amino]-1-{6-[(1-methylpiperidin-4-yl)amino]pyridin-2-yl}-1H,2H,3H-pyrazolo[3,4-d]pyrimidin-3-one (Compound 288)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 2-ethyl-6-[(1-methyl-1H-indazol-5-yl)amino]-1-{6-[(piperidin-4-yl)amino]pyridin-2-yl}-1H,2H,3H-pyrazolo[3,4-d]pyrimidin-3-one; trifluoroacetic acid (39 mg). Yield: 24 mg TFA salt (60%) as a powder.

### Example 221. Synthesis of 2-allyl-1-[6-(1-methyl-4-piperidyloxy)-2-pyridyl]-6-[p-(2,2,2-trifluoroethoxy)phenylamino]-1,2-dihydro-3H-1,2,5,7-tetraazainden-3-one (Compound 291)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 2-allyl-1-[6-(4-piperidyloxy)-2-pyridyl]-6-[p-(2,2,2-trifluoroethoxy)phenylamino]-1,2-dihydro-3H-1,2,5,7-tetraazainden-3-one (200 mg), Yield 100 mg.

### Example 222. Synthesis of 1-{6-[(1-methylpiperidin-4-yl)oxy]pyridin-2-yl}-6-[(5-methylpyridin-3-yl)amino]-2-(prop-2-en-1-yl)-1H,2H,3H-pyrazolo[3,4-d]pyrimidin-3-one (Compound 292)

### tert-butyl 4-[(6-{6-[(5-methylpyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (100 mg) and 3-bromo-5-methylpyridine (1.5 equiv.). Yield: 85 mg, purity 60% (43%).

### 6-[(5-methylpyridin-3-yl)amino]-1-[6-(piperidin-4-yloxy)pyridin-2-yl]-2-(prop-2-en-1-yl)-1H,2H,3H-pyrazolo[3,4-d]pyrimidin-3-one

Trifluoroacetic acid was added to a stirred solution of tert-butyl 4-[(6-{6-[(5-methylpyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate (81 mg, 60% purity) in dry dichloromethane at room temperature. The resulting solution was stirred until LCMS indicated full conversion. The reaction mixture was concentrated and purified using reversed phase chromatography. The pure fractions were pooled and lyophilized to give the crude intermediate. Yield: 63 mg, 60% purity (95%).

### 1-{6-[(1-methylpiperidin-4-yl)oxy]pyridin-2-yl}-6-[(5-methylpyridin-3-yl)amino]-2-(prop-2-en-1-yl)-1H,2H,3H-pyrazolo[3,4-d]pyrimidin-3-one

This material was methylated using a method similar as that described for 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 6-[(5-methylpyridin-3-yl)amino]-1-[6-(piperidin-4-yloxy)pyridin-2-yl]-2-(prop-2-en-1-yl)-1H,2H,3H-pyrazolo[3,4-d]pyrimidin-3-one (63 mg, 60% purity). Yield: 18.3 mg (45%) as a powder.

### Example 223. Synthesis of 6-[(1-methyl-1H-pyrazol-4-yl)amino]-1-(6-{[(1S,4S,5S)-2-methyl-2-azabicyclo[2.2.2]octan-5-yl]oxy}pyridin-2-yl)-2-(prop-2-en-1-yl)-1H,2H,3H-pyrazolo[3,4-d]pyrimidin-3-one (Compound 294)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-{6-[(1S,4S,5S)-2-azabicyclo[2.2.2]octan-5-yloxy]pyridin-2-yl}-6-[(1-methyl-1H-pyrazol-4-yl)amino]-2-(prop-2-en-1-yl)-1H,2H,3H-pyrazolo[3,4-d]pyrimidin-3-one (107 mg, crude). Yield: 13.3 mg TFA salt (10%) as a powder.

### Example 224. Synthesis of 6-[(1-methyl-1H-pyrazol-4-yl)amino]-1-(6-{[(1S,4S,5R)-2-methyl-2-azabicyclo[2.2.2]octan-5-yl]oxy}pyridin-2-yl)-2-(prop-2-en-1-yl)-1H,2H,3H-pyrazolo[3,4-d]pyrimidin-3-one (Compound 295)

This compound was made using a similar method as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one using 1-{6-[(1S,4S,5R)-2-azabicyclo[2.2.2]octan-5-yloxy]pyridin-2-yl}-6-[(1-methyl-1H-pyrazol-4-yl)amino]-2-(prop-2-en-1-yl)-1H,2H,3H-pyrazolo[3,4-d]pyrimidin-3-one (105 mg). Yield: 20.5 mg (19%) as a powder.

### Example 225. Synthesis of 2-allyl-1-(6-(piperidin-4-yloxy)pyridin-2-yl)-6-(m-tolylamino)-1,2-dihydro-3H-pyrazolo[3,4-d]pyrimidin-3-one (Compound 296)

### tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (1 g, 2.006 mmol) in 20 ml DCM, mCPBA (0.692 g, 4.01 mmol) was added at 0 °C. The reaction was stirred at rt for 2 h. The progress of the reaction was monitored by TLC, after completion of the reaction, the reaction mixture was quenched with 10% bicarbonate solution in water (50 mL) and extracted with DCM (250 mL). The organic layer was washed with water (250 ml), brine solution (250 ml) and dried over Na₂SO₄, filtered and concentrated under reduced pressure to give tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (1, 1.9 g).

### tert-butyl 4-((6-(2-allyl-3-oxo-6-(m-tolylamino)-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (600 mg, 1.131 mmol) in Acetic acid (10 ml), m-toluidine (2, 121 mg, 1.131 mmol) was added and the reaction mixture was stirred at RT for 16 h. The progress of the reaction was monitored by TLC. The reaction mixture was concentrated under vacuum. The crude residue was neutralized by aqueous sodium bicarbonate solution (50 ml) and extracted with 10% methanol-DCM. The combined organic layers were dried over sodium sulfate and concentrated under vacuum. The crude product was purified by flash column chromatography (silica-gel, mesh size 60-120) using ethyl acetate (100%) as an eluent to obtain desired product tert-butyl 4-((6-(2-allyl-3-oxo-6-(m-tolylamino)-2,3-dihydro-1H-pyrazolo[3,4-d]pyrimidin-1-yl)pyridin-2-yl)oxy)piperidine-1-carboxylate (400 mg, 0.572 mmol, 50.6% yield) as a yellow solid.

### 2-allyl-1-(6-(piperidin-4-yloxy)pyridin-2-yl)-6-(m-tolylamino)-1,2-dihydro-3H-pyrazolo[3,4-d]pyrimidin-3-one

To a stirred solution of tert-butyl 4-((6-(2-allyl-3-oxo-6-(m-tolylamino)-2,3-dihydro-1H-pyrazolo[3,4-d]pyrimidin-1-yl)pyridin-2-yl)oxy)piperidine-1-carboxylate (200 mg, 0.359 mmol) in DCM (10 ml), 4M HCl in dioxane (12.91 mg, 0.359 mmol) was added and the reaction mixture was stirred at RT for 16 h. The progress of the reaction was monitored by TLC. The reaction mixture was concentrated under vacuum. The residue was purified by prep-HPLC column no-S/DC/ARD/LC/22/29, X-SELECT C18 (250*19*5U), 0.1% FA in H₂O. Pure fractions collected and lyophilized to get 2-allyl-1-(6-(piperidin-4-yloxy)pyridin-2-yl)-6-(m-tolylamino)-1,2-dihydro-3H-pyrazolo[3,4-d]pyrimidin-3-one (125 mg, 0.264 mmol, 73.7% yield) as an off white solid.

### Example 226. Synthesis of 2-allyl-6-(p-fluorophenylamino)-1-[6-(4-piperidyloxy)-2-pyridyl]-1,2-dihydro-3H-1,2,5,7-tetraazainden-3-one (Compound 304)

This compound was made using a similar method as described in the synthesis of 2-allyl-6-((4-chlorophenyl)amino)-1-(6-(piperidin-4-yloxy)pyridin-2-yl)-1,2-dihydro-3H-pyrazolo[3,4-d]pyrimidin-3-one. Yield 101 mg.

### Example 227. Synthesis of 2-ethyl-6-(1-methyl-1H-indazol-5-ylamino)-1-[6-(4-piperidyloxy)-2-pyridyl]-1,2-dihydro-3H-1,2,5,7-tetraazainden-3-one (Compound 306)

This compound was made using a similar method as described in the synthesis of 2-allyl-6-((4-chlorophenyl)amino)-1-(6-(piperidin-4-yloxy)pyridin-2-yl)-1,2-dihydro-3H-pyrazolo[3,4-d]pyrimidin-3-one. Yield 39 mg.

### Example 228. Synthesis of 2-ethyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (Compound 318)

### tert-butyl 4-((6-(2-ethyl-6-(methylsulfonyl)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-ethyl-6-(methylthio)-3-oxo-2,3-dihydro-1H-pyrazolo[3,4-d]pyrimidin-1-yl)pyridin-2-yl)oxy)piperidine-1-carboxylate (300 mg, 0.617 mmol) in DCM (10 ml) at 0 °C, was added mCPBA (319 mg, 1.295 mmol) and the resulting mixture was stirred at RT for 2 h. The progress of the reaction was monitored by TLC. After completion of the reaction, the reaction mixture was diluted with DCM (300 mL) and washed with sodium bicarbonate solution (2×150 mL). The combined organic layer was dried over Na₂SO₄, filtered and concentrated under reduced pressure to afford the crude tert-butyl 4-((6-(2-ethyl-6-(methylsulfonyl)-3-oxo-2,3-dihydro-1H-pyrazolo[3,4-d]pyrimidin-1-yl)pyridin-2-yl)oxy)piperidine-1-carboxylate (250 mg, 0.741 mmol, 84% yield) as pale yellow solid.

### tert-butyl 4-((6-(2-ethyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-ethyl-6-(methylsulfonyl)-3-oxo-2,3-dihydro-1H-pyrazolo[3,4-d]pyrimidin-1-yl)pyridin-2-yl)oxy)piperidine-1-carboxylate (400 mg, 0.771 mmol) in acetic acid (5 mL), was added 1-methyl-1H-benzo[d]imidazol-5-amine (114 mg, 0.771 mmol) at room temperature. The reaction mixture was stirred at rt for 16 h. The progress of the reaction was monitored by TLC. After completion of the reaction, the reaction mixture was concentrated under reduced pressure. The residue was basified with sodium bicarbonate and extracted with ethyl acetate (10 mL×2). The combined organic layer was dried over Na₂SO₄, filtered, and concentrated under reduced pressure. The resulting crude material was purified by flash chromatography (SiO₂/100-200 mesh; 10-15% methanol-DCM) to afford tert-butyl 4-((6-(2-ethyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (250 mg, 0.213 mmol, 27.7% yield) as a pale brown solid.

### 2-ethyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-ethyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (200 mg, 0.341 mmol) in 1,4-dioxane (2 mL), was added 4M HCl in 1,4-dioxane (0.5 mL) at 0 °C. The resulting reaction mixture was stirred at room temperature for 8 h. The progress of the reaction was monitored by TLC. After completion of the reaction, the reaction mixture was concentrated under reduced pressure. The resultant crude residue was purified by prep. X-Select C18 (19*250, 5U), Mobile phase A: 10 MM ammonium acetate in H₂O, Mobile phase B: acetonitrile) to give 2-ethyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (22 mg, 0.045 mmol, 12.60% yield) as an off white solid.

### Example 229. Synthesis of 2-allyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (Compound 319)

### tert-butyl 4-((6-(2-Allyl-6-(methylsulfonyl)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (200 mg, 0.401 mmol) in DCM (10 ml) at 0 °C, was added m-CPBA (208 mg, 0.842 mmol) and the resulting mixture was stirred at RT for 2 h. The progress of the reaction was monitored by TLC. After completion of the reaction, the reaction mixture was diluted with DCM (300 mL) and washed with sodium bicarbonate solution (2×150 mL). The combined organic layer was dried over Na₂SO₄, filtered and concentrated under reduced pressure to afford the crude tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (180 mg, 0.292 mmol, 72.7% yield) as pale yellow solid.

### tert-butyl 4-((6-(2-allyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (200 mg, 0.377 mmol) in acetic acid (5 mL) was added 1-methyl-1H-benzo[d]imidazol-5-amine (61.0 mg, 0.415 mmol) at room temperature. The reaction mixture was stirred at rt for 16 h. The progress of the reaction was monitored by TLC. After completion of the reaction, the reaction mixture was concentrated under reduced pressure. The residue was basified with sodium bicarbonate and extracted with ethyl acetate (10 mL×2). The combined organic layer was dried over Na₂SO₄, filtered, and concentrated under reduced pressure. The crude product was purified by flash chromatography (SiO₂/100-200 mesh; 5-8% methanol-DCM) to afford tert-butyl 4-((6-(2-allyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (250 mg, 0.230 mmol, 61.0% yield)) as a pale brown solid.

### 2-allyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-methyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (100 mg, 0.167 mmol) in 1,4-dioxane (2 mL), was added 4M HCl in 1,4-dioxane (0.5 mL) at 0 °C. The resulting reaction mixture was stirred at room temperature for 8 h. The progress of the reaction was monitored by TLC. After completion of the reaction, the reaction mixture was concentrated under reduced pressure. The residue was purified by prep. X-Select C18 (19*250, 5U), Mobile phase A: 10 MM ABC in H₂O, Mobile phase B: acetonitrile) to give 2-allyl-6-((1-methyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (25.5 mg, 0.051 mmol, 30.6% yield) as an off white solid.

### Example 230. Synthesis of 2-allyl-1-[6-(1-methyl-4-piperidyloxy)-2-pyridyl]-6-(6-quinolylamino)-1,2-dihydro-3H-1,2,5,7-tetraazainden-3-one (Compound 325)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using tert-butyl 4-[6-(2-allyl-6-amino-3-oxo-1,2-dihydro-3H-1,2,5,7-tetraazainden-1-yl)-2-pyridyloxy]-1-piperidinecarboxylate (100 mg) and 6-bromoquinoline (44.5 mg). Yield 5.9 mg.

### Example 231. Synthesis of 5-[(1-{6-[(1-methylpiperidin-4-yl)oxy]pyridin-2-yl}-3-oxo-2-(prop-2-en-1-yl)-1H,2H,3H-pyrazolo[3,4-d]pyrimidin-6-yl)amino]pyridine-3-carbonitrile (Compound 330)

### tert-butyl 4-[(6-{6-[(5-cyanopyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This intermediate was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (204 mg) and 5-bromopyridine-3-carbonitrile (2 equiv.). Yield: 235 mg, purity 80% (77%).

### 5-({3-oxo-1-[6-(piperidin-4-yloxy)pyridin-2-yl]-2-(prop-2-en-1-yl)-1H,2H,3H-pyrazolo[3,4-d]pyrimidin-6-yl}amino)pyridine-3-carbonitrile

Trifluoroacetic acid was added to a stirred solution of tert-butyl 4-[(6-{6-[(5-cyanopyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate (235 mg). Yield: 199 mg (crude).

### 5-[(1-{6-[(1-methylpiperidin-4-yl)oxy]pyridin-2-yl}-3-oxo-2-(prop-2-en-1-yl)-1H,2H,3H-pyrazolo[3,4-d]pyrimidin-6-yl)amino]pyridine-3-carbonitrile

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using 5-({3-oxo-1-[6-(piperidin-4-yloxy)pyridin-2-yl]-2-(prop-2-en-1-yl)-1H,2H,3H-pyrazolo[3,4-d]pyrimidin-6-yl}amino)pyridine-3-carbonitrile (170 mg, 80% purity). Yield: 48 mg FA salt (31%) as a pale-yellow powder.

### Example 232. Synthesis of 6-[(1-methyl-1H-indazol-5-yl)amino]-1-(6-{[(3R)-1-methylpiperidin-3-yl]oxy}pyridin-2-yl)-2-(prop-2-en-1-yl)-1H,2H,3H-pyrazolo[3,4-d]pyrimidin-3-one (Compound 331)

This compound was made using a similar method as described in the synthesis of 2-allyl-6-((4-chlorophenyl)amino)-1-(6-(piperidin-4-yloxy)pyridin-2-yl)-1,2-dihydro-3H-pyrazolo[3,4-d]pyrimidin-3-one using 6-[(1-methyl-1H-indazol-5-yl)amino]-1-{6-[(3R)-piperidin-3-yloxy]pyridin-2-yl}-2-(prop-2-en-1-yl)-1H,2H,3H-pyrazolo[3,4-d]pyrimidin-3-one; trifluoroacetic acid (115 mg). Yield: 94 mg TFA salt (80%) as a pale-yellow powder.

### Example 233. Synthesis of 2-allyl-6-((3-(methyl-d3)phenyl)amino)-1-(6-(piperidin-4-yloxy)pyridin-2-yl)-1,2-dihydro-3H-pyrazolo[3,4-d]pyrimidin-3-one (Compound 334) and 2-allyl-6-((3-(methyl-d3)phenyl)amino)-1-(6-((1-methylpiperidin-4-yl)oxy)pyridin-2-yl)-1,2-dihydro-3H-pyrazolo[3,4-d]pyrimidin-3-one (Compound 333)

### 1-bromo-3-(methyl-d3)benzene

To a stirred solution of 1,3-dibromobenzene (4 g, 16.96 mmol) in THF (20 ml) was added n-BuLi (21.19 ml, 33.9 mmol) at −78 °C under inert atmosphere. The mixture was then stirred for 30 min, followed by the addition of CD₃I (2.156 ml, 33.9 mmol). The mixture was stirred for 1 h at −78 °C. The progress of the reaction was monitored by TLC and GCMS. After completion of the reaction, the reaction was quenched with ice-cold water and extracted with ethyl acetate (2×100 mL). The combined organic layers were dried over anhydrous Na₂SO₄. The solvent was evaporated under reduced pressure to give 1-bromo-3-(methyl-d3)benzene (1.5 g, 8.62 mmol, 50.8% yield) as pale yellow gummy liquid.

### tert-butyl (3-(methyl-d3)phenyl) carbamate

To a stirred solution of 1-bromo-3-(methyl-d3)benzene (400 mg, 2.298 mmol), tert-butyl carbamate 3538 mg, 4.60 mmol), Cs₂CO₃ (2246 mg, 6.89 mmol) and Xphos (219 mg, 0.460 mmol) in 1,4-dioxane (40 ml), were added. The mixture was degassed for 20 minutes at 25 °C. Then was added Pd(OAc)₂ (103 mg, 0.460 mmol) and the mixture was stirred at 100 °C for 16 h. The progress of the reaction was monitored by TLC and LCMS. After completion of the reaction, the reaction mixture was filtered using a sintered funnel, and collected fractions were concentrated under reduced pressure to give crude compound, which was purified by flash column chromatography (silica mesh 100-200 at eluent of 1-10% ethyl acetate and hexane) to give the pure compound tert-butyl (3-(methyl-d3)phenyl) carbamate (200 mg, 0.846 mmol, 36.8% yield) as yellow solid.

### Preparation of 3-(methyl-d3)aniline

To a stirred solution of tert-butyl (3-(methyl-d3)phenyl) carbamate (200 mg, 0.951 mmol) in dioxane (2 mL) was added 4M HCl in dioxane (0.3 mL, 41.6 mg, 1.141 mmol) at 0 °C and under inert atmosphere. The mixture was stirred at 25 °C for 16 h. The progress of the reaction was monitored by TLC and LCMS. After completion of the reaction, the reaction mixture was concentrated under reduced pressure, and the residue was quenched with aq. sodium bicarbonate solution (10 mL) and extracted with EtOAc (2×50 mL). The combined organic layers was dried over anhydrous Na₂SO₄, evaporated under reduced pressure to give 3-(methyl-d3)aniline (90 mg, 0.694 mmol, 73.0% yield) as a brown liquid.

### tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate

To the stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (500 mg, 1.003 mmol) in DCM (5 ml) was added m-CPBA (346 mg, 2.006 mmol) at room temperature under inert atmosphere, then reaction mixture was allowed to stir at rt for 2 h. The progress of the reaction was monitored by TLC and LCMS. After completion of the reaction, the reaction mixture was quenched with aq. sodium bicarbonate solution (50 mL) and extracted with DCM. The combined organic layers was dried over Na₂SO₄ and concentrated under reduced pressure to give tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (450 mg, 0.806 mmol, 80% yield) as an off-white solid. This crude compound was taken for the next step without any further purification.

### tert-butyl 4-((6-(2-allyl-6-((3-(methyl-d3)phenyl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (450 mg, 0.848 mmol) in acetic acid (3 ml), was added 3-(methyl-d3)aniline (93 mg, 0.848 mmol) at RT. The reaction mixture was allowed to stir at room temperature for 16 h. The progress of the reaction was monitored by TLC and LCMS. After completion of the reaction, the solvent was evaporated under reduced pressure, and the residue was quenched with aq. sodium bicarbonate solution (100 mL) and extracted with 5% MeOH in DCM. The combined organic layers were dried over anhydrous Na₂SO₄, filtered, and the filtrate was concentrated under reduced pressure to give a crude compound. This crude compound was purified by column chromatography (silica mesh 100-200 at eluent of 1-10% MeOH and DCM) to give tert-butyl 4-((6-(2-allyl-6-((3-(methyl-d3)phenyl)amino)-3-oxo-2,3-dihydro-1H-pyrazolo[3,4-d]pyrimidin-1-yl)pyridin-2-yl)oxy)piperidine-1-carboxylate (150 mg, 0.268 mmol, 31.5% yield) as an off-white solid.

### 2-allyl-6-((3-(methyl-d3)phenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((3-((methyl-d3)phenyl)amino)-3-oxo-2,3-dihydro-1H-pyrazolo[3,4-d]pyrimidin-1-yl)pyridin-2-yl)oxy)piperidine-1-carboxylate (500 mg, 0.892 mmol) in 1,4-dioxane, was added 4M HCl in dioxane (0.3 mL, 39.0 mg, 1.070 mmol) at 0 °C and under inert atmosphere. The mixture was stirred at 25 °C for 16 h. The progress of the reaction was monitored by TLC and LCMS. After completion of the reaction, the reaction mixture was concentrated under reduced pressure, and the crude compound was washed with n-hexane, followed by purification by prep HPLC (Column: X-select CSH C18, Mobile phase A: Water (with 0.1% AA), Mobile phase B: acetonitrile, Flow rate-15.0 mL/Min, Rt-12.8) to give 2-allyl-6-((3-(methyl-d3)phenyl)amino)-1-(6-(piperidin-4-yloxy)pyridin-2-yl)-1,2-dihydro-3H-pyrazolo[3,4-d]pyrimidin-3-one (102 mg, 0.221 mmol, 24.83% yield) as a white solid.

### 2-allyl-6-((3-(methyl-d3)phenyl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((3-(methyl-d3)phenyl)amino)-1-(6-(piperidin-4-yloxy)pyridin-2-yl)-1,2-dihydro-3H-pyrazolo[3,4-d]pyrimidin-3-one (290 mg, 0.630 mmol) in THF (2 ml) was added 20% aq. formaldehyde (255 mg, 3.15 mmol), followed by the addition of STAB (400 mg, 1.889 mmol) portion-wise. The progress of the reaction was monitored by TLC and LCMS. After completion of the reaction, the reaction mixture was quenched with TFA followed by NH₃ in methanol. The mixture was extracted with 10% MeOH in DCM. The combined organic layers were dried over anhydrous sodium sulfate, filtered and concentrated under reduced pressure to afford crude compound which was purified by Prep HPLC (Column: X-select CSH C18, Mobile phase A: Water (with 0.1% AA), Mobile phase B: acetonitrile, Flow rate-15.0 mL/Min, Rt-12.8) to give 2-allyl-6-((3-(methyl-d3)phenyl)amino)-1-(6-((1-methylpiperidin-4-yl)oxy)pyridin-2-yl)-1,2-dihydro-3H-pyrazolo[3,4-d]pyrimidin-3-one (86 mg, 0.181 mmol, 28.8% yield) as a white solid.

### Example 234. Synthesis of 2-methyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (Compound 336) and 2-methyl-6-((1-methyl-1H-indol-5-yl)amino)-1-(6-((1-methylpiperidin-4-yl)oxy)pyridin-2-yl)-1,2-dihydro-3H-pyrazolo[3,4-d]pyrimidin-3-one (Compound 360)

### tert-butyl 4-((6-(2-methyl-6-(methylthio)-3-oxo-2,3-dihydro-1H-pyrazolo[3,4-d]pyrimidin-1-yl)pyridin-2-yl)oxy)piperidine-1-carboxylate

A stirred solution of tert-butyl 4-((6-bromopyridin-2-yl)oxy)piperidine-1-carboxylate (1 g, 2.80 mmol), 2-methyl-6-(methylthio)-1,2-dihydro-3H-pyrazolo[3,4-d]pyrimidin-3-one (0.549 g, 2.80 mmol), K₂CO₃ (1.161 g, 8.40 mmol) and N,N-Dimethyl ethylenediamine (0.247 g, 2.80 mmol) in 1,4-dioxane (15 ml) was degassed for 20 min at rt under inert atmosphere. Then was added CuI (0.532 g, 2.80 mmol) and the mixture was stirred at 100 °C for 16 h. The progress of the reaction was monitored by TLC and LCMS. After completion of the reaction, the reaction mixture was filtered through celite and washed with ethyl acetate (2×100 mL). The collected organic layer was concentrated under reduced pressure to give the crude compound which was purified by column chromatography (SiO₂/230-400 mesh; 50-60% EtOAc in hexane) to give tert-butyl 4-((6-(2-methyl-6-(methylthio)-3-oxo-2,3-dihydro-1H-pyrazolo[3,4-d]pyrimidin-1-yl)pyridin-2-yl)oxy)piperidine-1-carboxylate (0.9 g, 1.847 mmol, 66.0% yield) as an off-white solid.

### tert-butyl 4-((6-(2-methyl-6-(methylsulfonyl)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-methyl-6-(methylthio)-3-oxo-2,3-dihydro-1H-pyrazolo[3,4-d]pyrimidin-1-yl)pyridin-2-yl)oxy)piperidine-1-carboxylate (1 g, 2.116 mmol) in DCM (10 ml) was added mCPBA (0.548 g, 3.17 mmol) at 0 °C under inert atmosphere. Then, the reaction was stirred at rt for 2 h. The progress of the reaction was monitored by TLC and LCMS. After completion of the reaction, aq. NaHCO₃ (100 mL) was added, and the mixture was extracted with ethyl acetate (2×150 mL). The combined organic layers was washed with brine solution (150 ml) and dried over Na₂SO₄, filtered and concentrated under reduced pressure to give tert-butyl 4-((6-(2-methyl-6-(methylsulfonyl)-3-oxo-2,3-dihydro-1H-pyrazolo[3,4-d]pyrimidin-1-yl)pyridin-2-yl)oxy)piperidine-1-carboxylate (900 mg, 1.463 mmol, 69.1% yield) as a yellow solid. This crude compound was taken as such for the next step without further purification.

### tert-butyl 4-((6-(2-methyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-methyl-6-(methylsulfonyl)-3-oxo-2,3-dihydro-1H-pyrazolo[3,4-d]pyrimidin-1-yl)-3,4-dihydropyridin-2-yl)oxy)piperidine-1-carboxylate (900 mg, 1.777 mmol) in acetonitrile (10 ml) was added 1-methyl-1H-indol-5-amine (312 mg, 2.132 mmol) at rt and the mixture was stirred at 70 °C for 16 h. The progress of the reaction was monitored by TLC and LCMS. After completion of the reaction, the reaction mixture was cooled to 0 °C. A solid was collected by filtration and washed with n-hexane (50 ml) and dried under vacuum to afford tert-butyl 4-((6-(2-methyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (700 mg, 0.994 mmol, 55.9% yield) as a brown colored solid.

### 2-methyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-methyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (550 mg, 0.964 mmol) in DCM (10 ml) was added 4M HCl in dioxane (1.205 ml, 4.82 mmol) at 0 °C and under inert atmosphere. The mixture was then stirred at rt for 16 h. The progress of the reaction was monitored by TLC and LCMS. After completion of the reaction, the solvent was evaporated under reduced pressure and the residue was washed with n-hexane to give the crude compound which was purified by prep HPLC (Column: X-select CSH C18, Mobile Phase A: Ammonium acetate in water, Mobile Phase B: acetonitrile, Flowrate: 15.0 mL/Min, Rt-10.8) to give 2-methyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (12 mg, 0.026 mmol, 2.65% yield) as a pale yellow solid.

### 2-methyl-6-((1-methyl-1H-indol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-methyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (50 mg, 0.106 mmol) in THF (10 ml) was added 37% formaldehyde (43.1 mg, 0.531 mmol) followed by the addition of STAB (67.6 mg, 0.319 mmol) at rt and the mixture was stirred for 1 h. The progress of the reaction was monitored by TLC and LCMS. After completion of the reaction, the reaction mixture was quenched with NH₃ in MeOH followed by TFA at rt and water. The mixture was extracted with 10% MeOH in DCM (2×100 mL). The combined organic layers was dried over anhydrous Na₂SO₄. The solvent was evaporated under reduced pressure to give the crude compound which was purified by prep HPLC (Column: X-select CSH C18, Mobile phase A: Water (with 0.1% AA), Mobile phase B: acetonitrile, Flow rate-15.0 mL/Min, Rt-12.8) to give 2-methyl-6-((1-methyl-1H-indol-5-yl)amino)-1-(6-((1-methylpiperidin-4-yl)oxy)pyridin-2-yl)-1,2-dihydro-3H-pyrazolo[3,4-d]pyrimidin-3-one (5.1 mg, 10.31 μmol, 9.71% yield) as an off-white solid.

### Example 235. Synthesis of 2-allyl-1-(6-((2,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (Compound 337) and 2-allyl-6-((1-methyl-1H-indazol-5-yl)amino)-1-(6-((1,2,2-trimethylpiperidin-4-yl)oxy)pyridin-2-yl)-1,2-dihydro-3H-pyrazolo[3,4-d]pyrimidin-3-one (Compound 343)

### tert-butyl 4-hydroxy-2,2-dimethylpiperidine-1-carboxylate

To a stirred solution of tert-butyl 2,2-dimethyl-4-oxopiperidine-1-carboxylate (500 mg, 2.200 mmol) in MeOH (10 mL) was added sodium borohydride (166 mg, 4.40 mmol) at 0 °C. The reaction was allowed to stir at 25 °C for 16 h. The progress of the reaction was monitored by TLC. After completion of the reaction, the reaction mixture was diluted with water and extracted with ethyl acetate (100 mL×2). The combined organic layer was dried over Na₂SO₄, filtered, and concentrated under reduced pressure to afford tert-butyl 4-hydroxy-2,2-dimethylpiperidine-1-carboxylate (500 mg, 2.093 mmol, 95% yield) as a colorless liquid.

### tert-butyl 4-((6-bromopyridin-2-yl)oxy)-2,2-dimethylpiperidine-1-carboxylate

To a stirred solution of tert-butyl 4-hydroxy-2,2-dimethylpiperidine-1-carboxylate (500 mg, 2.180 mmol) in THF (10 ml) was added NaH (60% in oil, 157 mg, 6.54 mmol). After 10 min at 25 °C, was added 2,6-dibromopyridine (3, 620 mg, 2.62 mmol). The reaction was allowed to stir at 25 °C for 2 h. The progress of the reaction was monitored by TLC and UPLC. After completion of the reaction, ice water was added, and the product was extracted with ethyl acetate. The combined organic layer was washed with brine, dried over anhydrous sodium sulfate, and concentrated under reduced pressure. The resulting crude material was purified by flash chromatography (SiO₂/100-200 mesh; 10-20% ethyl acetate/hexane) to afford tert-butyl 4-((6-bromopyridin-2-yl)oxy)-2,2-dimethylpiperidine-1-carboxylate (850 mg, 1.434 mmol, 65.8% yield) as an off-white solid.

### tert-butyl 4-((6-(2-allyl-6-(methylthio)-3-oxo-2,3-dihydro-1H-pyrazolo[3,4-d]pyrimidin-1-yl)pyridin-2-yl)oxy)-2,2-dimethylpiperidine-1-carboxylate

A stirred solution of tert-butyl 4-((6-bromopyridin-2-yl)oxy)-2,2-dimethylpiperidine-1-carboxylate (600 mg, 1.292 mmol), 2-allyl-6-(methylthio)-1,2-dihydro-3H-pyrazolo[3,4-d]pyrimidin-3-one (345 mg, 1.551 mmol, Potassium Carbonate (536 mg, 3.88 mmol) and N,N-dimethyl-ethylenediamine (228 mg, 2.58 mmol) in dioxane (10 ml) was degassed for 20 min at rt under inert atmosphere. Then CuI (114 mg, 0.600 mmol) was added and the mixture was stirred at 100 °C for 16 h. The progress of the reaction was monitored by UPLC. After completion of the reaction, the reaction mass was diluted with DCM and filtered through celite pad. The collected filtrate was concentrated under reduced pressure. The residue was purified by flash column chromatography (silica gel, 100-200 mesh size) using ethyl acetate-pet ether (10%-20%) as an eluent to obtain tert-butyl 4-((6-(2-allyl-6-(methylthio)-3-oxo-2,3-dihydro-1H-pyrazolo[3,4-d]pyrimidin-1-yl)pyridin-2-yl)oxy)-2,2-dimethylpiperidine-1-carboxylate (400 mg, 0.615 mmol, 47.6% yield) off white solid.

### tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2,2-dimethylpiperidine-1-carboxylate

To a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)-2,2-dimethylpiperidine-1-carboxylate (6, 400 mg, 0.760 mmol) in DCM (5 ml) at 0 °C was added mCPBA (262 mg, 1.519 mmol) and the mixture was stirred at RT for 2 h. The progress of the reaction was monitored by TLC and UPLC. After completion of the reaction, ice water was added and the mixture was extracted with DCM (100 mL×2). The combined organic layer was washed with aqueous sodium bicarbonate solution, dried over anhydrous sodium sulfate, and concentrated under reduced pressure to obtained tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2,2-dimethylpiperidine-1-carboxylate (410 mg, 0.491 mmol, 64.7% yield) as a pale yellow semi-solid.

### tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2,2-dimethylpiperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2,2-dimethylpiperidine-1-carboxylate (410 mg, 0.756 mmol) in acetonitrile (5 mL) was added 1-methyl-1H-indazol-5-amine (133 mg, 0.907 mmol) at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using ethyl acetate-hexane (40% to 50%) as an eluent to obtain tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2,2-dimethylpiperidine-1-carboxylate (425 mg, 0.506 mmol, 66.9% yield) as a brown solid.

### 2-allyl-1-(6-((2,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one

To a stirred solution of tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2,2-dimethylpiperidine-1-carboxylate (400 mg, 0.639 mmol) in 1,4-dioxane (5 mL), was added 4M HCl in 1,4-dioxane (2.0 mL) at 0 °C. The resulting reaction mixture was stirred at room temperature for 4 h. The progress of the reaction was monitored by TLC. After completion of the reaction, the reaction mixture was concentrated under reduced pressure. The crude residue was purified by prep. HPLC X-Select C18 (19*250, 5 mL), Mobile phase A: 10 MM AA in H2O, Mobile phase B: acetonitrile) to give 2-allyl-1-(6-((2,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (195.05 mg, 0.364 mmol, 56.9% yield) as an off white-solid.

### 2-allyl-6-((1-methyl-1H-indazol-5-yl)amino)-1-(6-((1,2,2-trimethylpiperidin-4-yl)oxy)pyridin-2-yl)-1,2-dihydro-3H-pyrazolo[3,4-d]pyrimidin-3-one

To a stirred solution of 2-allyl-1-(6-((2,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (450 mg, 0.856 mmol) in THF (10 mL) was added formaldehyde (0.382 ml, 5.14 mmol) at 25 °C. The reaction mixture stirred at 25 °C for 5 min. Then STAB (1089 mg, 5.14 mmol) was added portion-wise. After addition, the reaction mixture was stirred at 25 °C for 20 min. The progress of the reaction was monitored by TLC and LCMS. The reaction mixture was quenched with TFA, followed by NH₃ in MeOH, diluted with water and extracted with 10% MeOH in DCM (2×100 mL). The combined organic extracts was washed with NaHCO₃, dried over anhydrous Na₂SO₄, filtered, and concentrated under reduced pressure to afford crude compound, which was purified by prep. HPLC X-Select C18 (19*250, 5 mL), Mobile phase A: 10 MM AA in H₂O, Mobile phase B: acetonitrile) to give 2-allyl-6-((1-methyl-1H-indazol-5-yl)amino)-1-(6-((1,2,2-trimethylpiperidin-4-yl)oxy)pyridin-2-yl)-1,2-dihydro-3H-pyrazolo[3,4-d]pyrimidin-3-one (135.83 mg, 0.247 mmol, 28.8% yield) as an off-white solid.

### Example 236. Synthesis of 2-ethyl-6-[(1-methyl-1H-pyrazol-4-yl)amino]-1-{6-[(1-methylpiperidin-4-yl)oxy]pyridin-2-yl}-1H,2H,3H-pyrazolo[3,4-d]pyrimidin-3-one (Compound 341)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one using 2-ethyl-6-[m-(1-methyl-4-pyrazolyl)phenylamino]-1-[6-(4-piperidyloxy)-2-pyridyl]-1,2-dihydro-3H-1,2,5,7-tetraazainden-3-one (150 mg). Yield 77 mg.

### Example 237. Synthesis of 6-[(5-methoxypyridin-3-yl)amino]-1-{6-[(1-methylpiperidin-4-yl)oxy]pyridin-2-yl}-2-(prop-2-en-1-yl)-1H,2H,3H-pyrazolo[3,4-d]pyrimidin-3-one (Compound 342)

This compound was made using a similar method as described in the synthesis of 2-allyl-6-(2-methoxy-4-pyridylamino)-1-[6-(1-methyl-4-piperidyloxy)-2-pyridyl]-1,2-dihydro-3H-1,2,5,7-tetraazainden-3-one using 6-[(5-methoxypyridin-3-yl)amino]-1-[6-(piperidin-4-yloxy)pyridin-2-yl]-2-(prop-2-en-1-yl)-1H,2H,3H-pyrazolo[3,4-d]pyrimidin-3-one (198 mg). Yield: 157 mg TFA salt (62%) as a yellow powder.

### Example 238. Synthesis of 2-allyl-6-((3-methyl-1H-indazol-5-yl)amino)-1-(6-(piperidin-4-yloxy)pyridin-2-yl)-1,2-dihydro-3H-pyrazolo[3,4-d]pyrimidin-3-one (Compound 345) and 2-allyl-6-((3-methyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 355)

### tert-butyl 4-((6-(2-allyl-6-((3-methyl-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (1.0 g, 1.943 mmol) in acetonitrile (5 mL) 3-methyl-1H-indazol-5-amine (0.286 g, 1.943 mmol) was added at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The above crude was purified by flash column chromatography (silica gel, 100-200 mesh size) using ethyl acetate-hexane (40% to 50%) as an eluent to obtain tert-butyl 4-((6-(2-allyl-6-((3-methyl-1H-indazol-5-yl)amino)-3-oxo-2,3-dihydro-1H-pyrazolo[3,4-d]pyrimidin-1-yl)pyridin-2-yl)oxy)piperidine-1-carboxylate (1.0 g, 1.601 mmol, 82% yield) as a brown solid.

### 2-allyl-6-((3-methyl-1H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((3-methyl-1H-indazol-5-yl)amino)-3-oxo-2,3-dihydro-1H-pyrazolo[3,4-d]pyrimidin-1-yl)pyridin-2-yl)oxy)piperidine-1-carboxylate (1.0 g, 1.673 mmol) in 1,4-dioxane (5 mL), was added 4M HCl in 1,4-dioxane (2.0 mL) at 0 °C. The resulting reaction mixture was stirred at room temperature for 4 h. The progress of the reaction was monitored by TLC. After completion of the reaction, the reaction mixture was concentrated under reduced pressure. Of the crude material, 800 mg was taken for the next step without any further purification. The remaining 200 mg crude residue was purified by prep. HPLC X-Select C18 (19*250, 5 mL), Mobile phase A: 10 MM ABC in H₂O, Mobile phase B: acetonitrile) to give 2-allyl-6-((3-methyl-1H-indazol-5-yl)amino)-1-(6-(piperidin-4-yloxy)pyridin-2-yl)-1,2-dihydro-3H-pyrazolo[3,4-d]pyrimidin-3-one (0.100 g, 0.199 mmol, 11.89% yield) as an off white-solid.

### 2-allyl-6-((3-methyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((3-methyl-1H-indazol-5-yl)amino)-1-(6-(piperidin-4-yloxy)pyridin-2-yl)-1,2-dihydro-3H-pyrazolo[3,4-d]pyrimidin-3-one (800 mg, 1.608 mmol) in THF (10 mL) was added formaldehyde (20% Aq., 4 mL) at 25 °C. The reaction mixture was stirred at 25 °C for 5 min. Then, was added portion-wise STAB (1022 mg, 4.82 mmol). After the complete addition of STAB, the reaction mixture was stirred at 25 °C for 20 min. The reaction was monitored by TLC and LCMS. After completion of the reaction, the reaction mixture was quenched with TFA, followed by NH₃ in MeOH diluted with water and extracted with 10% MeOH in DCM (2×100 mL). The above combined organic extracts were washed with NaHCO₃, dried over anhydrous Na₂SO₄, filtered, and concentrated under reduced pressure. The resultant crude material was purified by prep. HPLC (ZORBAX 50*21 mm), Mobile phase A: 10 MM ABC in H₂O, Mobile phase B: acetonitrile) to give 2-allyl-6-((3-methyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (125 mg, 0.16 mmol, 10.2% yield) as an off-white solid.

### Example 239. Synthesis of 2-allyl-6-((1,3-dimethyl-1H-indazol-5-yl)amino)-1-(6-(piperidin-4-yloxy)pyridin-2-yl)-1,2-dihydro-3H-pyrazolo[3,4-d]pyrimidin-3-one (Compound 347) and 2-allyl-6-((1,3-dimethyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 346)

### tert-butyl 4-((6-(2-allyl-6-((1,3-dimethyl-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (1.0 g, 1.943 mmol) in acetonitrile (5 mL) was added 1,3-dimethyl-1H-indazol-5-amine (0.313 g, 1.943 mmol) at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using ethyl acetate-hexane (40% to 50%) as an eluent to obtain tert-butyl 4-((6-(2-allyl-6-((1,3-dimethyl-1H-indazol-5-yl)amino)-3-oxo-2,3-dihydro-1H-pyrazolo[3,4-d]pyrimidin-1-yl)pyridin-2-yl)oxy)piperidine-1-carboxylate (1.1 g, 1.068 mmol, 55.0% yield) as a brown solid.

### 2-allyl-6-((1,3-dimethyl-1H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1,3-dimethyl-1H-indazol-5-yl)amino)-3-oxo-2,3-dihydro-1H-pyrazolo[3,4-d]pyrimidin-1-yl)pyridin-2-yl)oxy)piperidine-1-carboxylate (1.0 g, 1.635 mmol) in 1,4-dioxane (5 mL), was added 4M HCl in 1,4-dioxane (2.0 mL) at 0 °C. The resulting reaction mixture was stirred at room temperature for 4 h. The progress of the reaction was monitored by TLC. After completion of the reaction, the reaction mixture was concentrated under reduced pressure. Of the resulting crude residue, 200 mg was purified by prep HPLC, and the remaining was taken to the next step without any further purification. HPLC X-Select C18 (19*250, 5 mL), Mobile phase A: 10 MM ABC in H₂O, Mobile phase B: acetonitrile) to get 2-allyl-6-((1,3-dimethyl-1H-indazol-5-yl)amino)-1-(6-(piperidin-4-yloxy)pyridin-2-yl)-1,2-dihydro-3H-pyrazolo[3,4-d]pyrimidin-3-one (0.070 g, 0.134 mmol, 8.18% yield) as an off white-solid.

### 2-allyl-6-((1,3-dimethyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1,3-dimethyl-1H-indazol-5-yl)amino)-1-(6-(piperidin-4-yloxy)pyridin-2-yl)-1,2-dihydro-3H-pyrazolo[3,4-d]pyrimidin-3-one (0.550 g, 1.075 mmol) in THF (10 mL) was added formaldehyde (0.214 ml, 2.150 mmol) at 25 °C. The reaction mixture was stirred at 25 °C for 5 min. Then, STAB (0.456 g, 2.150 mmol) was added portion-wise. After the complete addition of STAB, the reaction mixture was stirred at 25 °C for 20 min. The progress of the reaction was monitored by TLC and LCMS. The reaction mixture was quenched with TFA, followed by NH₃ in MeOH diluted with water and extracted with 10% MeOH in DCM (2×100 mL). The combined organic extracts were washed with NaHCO₃, dried over anhydrous Na₂SO₄, filtered, and concentrated under reduced pressure. The residue was purified by prep. HPLC (SHIMPACK C18 (250*19.5 mm), Mobile phase A: 10 MM ABC in H₂O, Mobile phase B: acetonitrile) to give 2-allyl-6-((1,3-dimethyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (0.160 g, 0.300 mmol, 27.9% yield) as an off-white solid.

### Example 240. Synthesis of 2-allyl-6-((1-methyl-1H-indazol-5-yl)amino)-1-(6-((1-(oxetan-3-yl)piperidin-4-yl)oxy)pyridin-2-yl)-1,2-dihydro-3H-pyrazolo[3,4-d]pyrimidin-3-one (Compound 349)

To a solution of 2-allyl-6-((1-methyl-1H-indazol-5-yl)amino)-1-(6-(piperidin-4-yloxy)pyridin-2-yl)-1,2-dihydro-3H-pyrazolo[3,4-d]pyrimidin-3-one (0.2 g, 0.402 mmol) in 1,2-DCE (10 ml) was added oxetan-3-one (0.058 g, 0.804 mmol) and the mixture was stirred at 60 °C for 1 h, STAB (0.128 g, 0.603 mmol) was added at room temperature and the mixture stirred for 16 h. The progress of the reaction was monitored by TLC and LCMS. After completion of the reaction, the reaction mixture was quenched with ice cold water (100 mL) and extracted with DCM (2×100 mL). The combined organic layers were dried over anhydrous Na₂SO₄, concentrated under reduced pressure to give the crude compound, which was purified by column chromatography (X Select C-8, 19×250, Mobile phase A: 10 mm ABC in Water, Mobile phase B: acetonitrile) to give 2-allyl-6-((1-methyl-1H-indazol-5-yl)amino)-1-(6-((1-(oxetan-3-yl)piperidin-4-yl)oxy)pyridin-2-yl)-1,2-dihydro-3H-pyrazolo[3,4-d]pyrimidin-3-one (180 mg, 0.321 mmol, 53.2% yield).

### Example 241. Synthesis of 2-ethyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (Compound 353)

### tert-butyl 4-((6-(2-ethyl-6-(methylthio)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-bromopyridin-2-yl)oxy)piperidine-1-carboxylate (700 mg, 1.959 mmol) in 1,4-dioxane (20 ml) were added 2-ethyl-6-(methylthio)-1,2-dihydro-3H-pyrazolo[3,4-d]pyrimidin-3-one (412 mg, 1.959 mmol), K₂CO₃ (812 mg, 5.88 mmol), N,N-dimethyl-ethylenediamine (173 mg, 1.959 mmol) and the mixture was degassed for 20 min at rt under inert atmosphere. Then was added CuI (373 mg, 1.959 mmol) at rt and the mixture was again degassed for 5 min. The reaction mixture was stirred at 110 °C for 16 h. The progress of the reaction was monitored by TLC and LCMS. After completion of the reaction, the reaction mixture was filtered through celite and washed with ethyl acetate (2×100 mL). The filtrate was collected and concentrated under reduced pressure to give the crude compound which was purified by column chromatography (silica gel, 100-200 mesh size: using ethyl acetate-pet ether 40%-60%) to give tert-butyl 4-((6-(2-ethyl-6-(methylthio)-3-oxo-2,3-dihydro-1H-pyrazolo[3,4-d]pyrimidin-1-yl)pyridin-2-yl)oxy)piperidine-1-carboxylate (900 mg, 1.794 mmol, 92% yield).

### tert-butyl 4-((6-(2-ethyl-6-(methylsulfonyl)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-ethyl-6-(methylthio)-3-oxo-2,3-dihydro-1H-pyrazolo[3,4-d]pyrimidin-1-yl)pyridin-2-yl)oxy)piperidine-1-carboxylate (0.9 g, 1.850 mmol) in DCM (10 ml) was added mCPBA (0.479 g, 2.77 mmol) at 0 °C under inert atmosphere. The reaction mixture was stirred at rt for 2 h. The progress of the reaction was monitored by TLC and LCMS. After completion of the reaction, aq. NaHCO₃ (150 mL) was added and the mixture was extracted with ethyl acetate (2×150 mL). The combined organic layers was washed with brine solution (150 ml), dried over Na₂SO₄, filtered, and concentrated under reduced pressure to give tert-butyl 4-((6-(2-ethyl-6-(methylsulfonyl)-3-oxo-2,3-dihydro-1H-pyrazolo[3,4-d]pyrimidin-1-yl)pyridin-2-yl)oxy)piperidine-1-carboxylate (0.9 g, 1.666 mmol, 90% yield) as a yellow solid. This crude compound, as such, is taken for the next step without any further purification.

### tert-butyl 4-((6-(2-ethyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-ethyl-6-(methylsulfonyl)-3-oxo-2,3-dihydro-1H-pyrazolo[3,4-d]pyrimidin-1-yl)-3,4-dihydropyridin-2-yl)oxy)piperidine-1-carboxylate (0.9 g, 1.729 mmol) in ACN (20 ml) was added 1-methyl-1H-indol-5-amine (0.303 g, 2.075 mmol) at rt under inert atmosphere. Then the reaction mixture was stirred at 70 °C for 16 h. The progress of the reaction was monitored by TLC and LCMS. After completion of the reaction, the reaction mixture was concentrated under reduced pressure. Then was added ice cold water (100 mL). The mixture was extracted with 10% MeOH in DCM (2×250 mL). The combined organic layers were dried over anhydrous Na₂SO₄, filtered and concentrated under reduced pressure to afford crude compound which was purified by flash column chromatography (silica-gel, mesh size 60-120: using ethyl acetate in n-hexane 70-80%) to obtain tert-butyl 4-((6-(2-ethyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (0.9 g, 1.509 mmol, 87% yield) as a white solid.

### 2-ethyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-ethyl-6-((1-methyl-1H-indol-5-yl)amino)-3-oxo-2,3-dihydro-1H-pyrazolo[3,4-d]pyrimidin-1-yl)pyridin-2-yl)oxy)piperidine-1-carboxylate (100 mg, 0.171 mmol) in MeOH (5 ml) was added oxalyl chloride (65.1 mg, 0.513 mmol) at 0 °C under inert atmosphere. Then, the reaction mixture was stirred at rt for 2 h. The progress of the reaction was monitored by TLC and LCMS. After completion of the reaction, reaction mixture was concentrated under reduced pressure to get crude material which was purified by reverse phase column chromatography to afford pure compound 2-ethyl-6-((1-methyl-1H-indol-5-yl)amino)-1-(6-(piperidin-4-yloxy)pyridin-2-yl)-1,2-dihydro-3H-pyrazolo[3,4-d]pyrimidin-3-one (15.6 mg, 0.032 mmol, 18.82% yield).

### Example 242. Synthesis of 2-allyl-6-((1-isobutyl-1H-indazol-5-yl)amino)-1-(6-(piperidin-4-yloxy)pyridin-2-yl)-1,2-dihydro-3H-pyrazolo[3,4-d]pyrimidin-3-one (Compound 356) and 2-allyl-6-((1-isobutyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 394)

### 1-isobutyl-5-nitro-1H-indazole

To a stirred solution of 1-iodo-2-methylpropane (0.714 mL, 6.13 mmol) and 5-nitro-1H-indazole (500 mg, 3.06 mmol) in DMF (12 mL) was added sodium hydride (60% in oil) (88 mg, 3.68 mmol) at 0 °C and the mixture was stirred for 16 h at room temperature. The progress of the reaction was monitored by TLC. After completion of the reaction, the reaction mixture was quenched with ice-cold water, and the precipitate obtained was filtered. The precipitate was then dissolved in DCM and purified by flash column chromatography (silica gel, 60-120 using ethyl acetate-hexane (20 to 70%) as an eluent to obtain 1-isobutyl-5-nitro-1H-indazole (0.155 g, 0.693 mmol, 22.61% yield) as an orange solid.

### 1-isobutyl-1H-indazol-5-amine

To a degassed solution of 1-isobutyl-5-nitro-1H-indazole (300 mg, 1.368 mmol) in MeOH (10 ml) was added 10% Pd/C (291 mg, 2.74 mmol) under an inert atmosphere. Then, the reaction mixture was stirred under a hydrogen atmosphere using hydrogen bladder pressure at room temperature for 16 hours. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure (bath temperature: 49 °C) to afford 1-isobutyl-1H-indazol-5-amine (290 mg, 1.165 mmol, 85% yield) as an orange solid, which was taken into the next step without further purification.

### tert-butyl 4-((6-(2-allyl-6-((1-isobutyl-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (600 mg, 1.166 mmol) in acetonitrile (10 mL) was added 1-isobutyl-1H-indazol-5-amine (276 mg, 1.457 mmol) at room temperature. The reaction mixture was subjected to stirring at 70 °C for 16 h. The progress of the reaction was monitored by LCMS. After the completion of the reaction, the reaction mixture was concentrated under reduced pressure. The crude product was purified by flash column chromatography (silica gel, 100-200) using ethyl acetate-hexane (50 to 100%) as an eluent to obtain tert-butyl 4-((6-(2-allyl-6-((1-isobutyl-1H-indazol-5-yl)amino)-3-oxo-2,3-dihydro-1H-pyrazolo[3,4-d]pyrimidin-1-yl)pyridin-2-yl)oxy)piperidine-1-carboxylate (310 mg, 0.359 mmol, 30.8% yield) as a red colored solid.

### 2-allyl-6-((1-isobutyl-1H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-isobutyl-1H-indazol-5-yl)amino)-3-oxo-2,3-dihydro-1H-pyrazolo[3,4-d]pyrimidin-1-yl)pyridin-2-yl)oxy)piperidine-1-carboxylate (310 mg, 0.354 mmol) in 1,4-dioxane (10 mL), was added 4M HCl in 1,4-dioxane (5.0 mL) at 0 °C. The resulting reaction mixture was stirred at room temperature for 4 h. The progress of the reaction was monitored by TLC. After completion of the reaction, the reaction mixture was concentrated under reduced pressure. The crude residue was purified by prep-HPLC Shimpack C18 (20*250) 5u, Mobile phase A: 10 mM Ammonium Bi-Carbonate in water, Mobile phase B: acetonitrile) to give 2-allyl-6-((1-isobutyl-1H-indazol-5-yl)amino)-1-(6-(piperidin-4-yloxy)pyridin-2-yl)-1,2-dihydro-3H-pyrazolo[3,4-d]pyrimidin-3-one (372 mg, 0.494 mmol, 98% yield) as an off white solid.

### 2-allyl-6-((1-isobutyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-isobutyl-1H-indazol-5-yl)amino)-1-(6-(piperidin-4-yloxy)pyridin-2-yl)-1,2-dihydro-3H-pyrazolo[3,4-d]pyrimidin-3-one (350 mg, 0.649 mmol) in THF (10 mL) was added 20% aq. formaldehyde (7 mL) at 25 °C. Then the reaction mixture was stirred at 25 °C for 5 min. sodium triacetoxyborohydride (275 mg, 1.297 mmol) was added portion-wise; after the complete addition of sodium triacetoxyborohydride, the reaction mixture was stirred at 25 °C for 20 min. The progress of the reaction was monitored by TLC and LCMS. The reaction mixture was quenched with TFA followed by NH₃ in MeOH, after which it was diluted with water and extracted with 10% MeOH in DCM (2×100 mL). The combined organic extract was washed with NaHCO₃, dried over anhydrous Na₂SO₄, filtered, and concentrated under reduced pressure to afford crude compound. The crude was purified by PREP-HPLC (xtim: xtimate c18, Mobile phase A: 0.1% FA in H₂O, Mobile phase B: acetonitrile) to give 2-allyl-6-((1-isobutyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (118 mg, 0.211 mmol, 32.5% yield) as a white fluffy solid.

### Example 243. Synthesis of rel-(R)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (Compound 357), rel-(R)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one (Compound 373), rel-(S)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (Compound 358), and rel-(S)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one (Compound 374)

### tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)azepane-1-carboxylate (4.6 g, 8.45 mmol) in AcOH (30 ml) was added 1-methyl-1H-indazol-5-amine (1.492 g, 10.14 mmol) and the mixture was allowed to stir for 3 h. The progress of the reaction was monitored by TLC and UPLC. After completion of the reaction, the solvent was evaporated under reduced pressure. The residue was quenched with aq. NaHCO₃ solution (100 mL) and extracted with 10% MeOH in DCM (2×200 mL). The combined organic extracts was washed with brine (100 mL), dried over anhydrous sodium sulfate, filtered, and concentrated under reduced pressure to afford crude compound; which was purified by column chromatography (silica mesh 100-200 at eluent of 40-50% ethyl acetate and hexane) to give tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate (1.2 g, 1.962 mmol, 23.23% yield) as an off-white solid. This racemate was separated by chiral SFC (I-CELLULOSE-Z, 0.5% IPAm in ACN-IPA-60-40, Oven Temperature: 40, Column Position: 4, BPR Pressure: 102.0 kg/cm²) to give the two enantiomers:

First eluting enantiomer: rel-tert-butyl (R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate.

Second eluting enantiomer rel-tert-butyl(S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate.

### rel-(R)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one

To a stirred solution of rel-tert-butyl (R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate (first eluting enantiomer, 300 mg, 0.490 mmol) in dioxane (5 ml) was added 4M HCl in dioxane (0.2 mL, 0.82 mmol) at 0 °C under inert atmosphere. The mixture was stirred for 16 h. The progress of the reaction was monitored by TLC and LCMS. After completion of the reaction, solvent was evaporated under reduced pressure to give rel-(R)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (200 mg, 0.391 mmol, 95.6% yield) as off-white solid. 30 mg of the above compound was taken and purified by prep HPLC (Column: X-SELECT CSH C18, Mobile phase A: Water (with 10 mM ABC), Mobile phase B: acetonitrile, Flow rate-15.0 mL/Min, Rt-10.8) to give the pure compound (14.71 mg). Chiral HPLC: 100.00%, tR=5.27 min.

### Preparation of rel-(R)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one

To a stirred solution of rel-(R)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (230 mg, 0.450 mmol) in THF (3 ml) was added 37% formaldehyde (0.2 mL, 2.248 mmol) followed by the addition of STAB (286 mg, 1.349 mmol) at rt. The mixture was stirred for 1 h. The progress of the reaction was monitored by TLC and LCMS. After completion of the reaction, the reaction mixture was quenched with NH₃ in MeOH followed by TFA at rt. The mixture was diluted with water and extracted with 10% MeOH in DCM (2×50 mL). The combined organic layers were dried over anhydrous Na₂SO₄. The solvent was evaporated under reduced pressure to give the crude compound which was purified by prep HPLC (Column: X-select CSH C18, Mobile phase A: Water (with 0.1% AA), Mobile phase B: acetonitrile, Flow rate-15.0 mL/Min, Rt-12.8) to give rel-(R)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one (37.34 mg, 0.071 mmol, 15.80% yield) as a white solid. Chiral HPLC: 96.81%, tR=5.50 min.

### Preparation of rel-(S)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one

To a stirred solution of rel-tert-butyl(S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)azepane-1-carboxylate (Second eluting enantiomer, 350 mg, 0.572 mmol) in dioxane (5 ml) was added 4M HCl in dioxane (0.15 mL, 0.572 mmol) at 0 °C under inert atmosphere. The mixture was stirred for 16 h. The progress of the reaction was monitored by TLC and LCMS. After completion of the reaction, the solvent was evaporated under reduced pressure to give rel-(S)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (260 mg, 0.508 mmol, 89.00% yield) as off-white solid. 30 mg of the above compound was taken and purified by prep HPLC (Column: X-SELECT CSH C18, Mobile phase A: Water (with 10 MM ABC), Mobile phase B: acetonitrile, Flow rate-15.0 mL/Min, Rt-10.8) to give the pure compound (12.11 mg). Chiral HPLC: 100.00%, tR=5.64 min.

### rel-(S)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one

To a stirred solution of rel-(S)-2-allyl-1-(6-(azepan-4-yloxy)pyridin-2-yl)-6-((1-methyl-1H-indazol-5-yl)amino)-1,2-dihydro-3H-pyrazolo[3,4-d]pyrimidin-3-one (345 mg, 0.674 mmol) in THF (5 ml) was added 37% formaldehyde (0.4 mL, 3.37 mmol) followed by the addition of STAB (429 mg, 2.023 mmol). The mixture was stirred at rt for 1 h. The progress of the reaction was monitored by TLC and LCMS. After completion of the reaction, the reaction mixture was quenched with methanolic ammonia 2 molar solution, followed by TFA at rt. The mixture was diluted with water and extracted with 10% MeOH in DCM (2×50 mL). The combined organic layers were dried over anhydrous Na₂SO₄. The solvent was evaporated under reduced pressure to give the crude compound which was purified by prep HPLC (Column: X-select CSH C18, Mobile phase A: Water (with 0.1% AA), Mobile phase B: acetonitrile, Flow rate-15.0 mL/Min, Rt-12.8) to give rel-(S)-2-allyl-6-((1-methyl-1H-indazol-5-yl)amino)-1-(6-((1-methylazepan-4-yl)oxy)pyridin-2-yl)-1,2-dihydro-3H-pyrazolo[3,4-d]pyrimidin-3-one (103.24 mg, 0.196 mmol, 29.1% yield) as a white solid. Chiral HPLC: 95.17%, tR=6.31 min.

### Example 244. Synthesis of 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one (Compound 359)

### tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (800 mg, 1.508 mmol) in AcOH (5 ml), was added 1-methyl-1H-indazol-6-amine (244 mg, 1.658 mmol) at rt under inert atmosphere. The mixture was stirred at rt for 16 h. The progress of the reaction was monitored by TLC and LCMS. After completion of the reaction, the reaction mixture was distilled, and residual solvent was neutralized with aq. NaHCO₃ (50 mL) and the mixture was extracted with DCM (2×250 mL). The combined organic layers were washed with brine solution (50 ml) and dried over anhydrous Na₂SO₄, filtered and concentrated under reduced pressure to give the crude compound which was purified by flash column chromatography (silica gel, 100-200 mesh size: ethyl acetate in hexane 60-80%) to give tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate (700 mg, 0.972 mmol, 64.5% yield) as an off-white solid.

### 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate (860 mg, 1.439 mmol) in 1,4-dioxane (5 ml) was added 4M HCl in dioxane (0.4 mL, 1.439 mmol) at 0 °C under inert atmosphere. Then, the resulting reaction mixture was stirred at room temperature for 16 h. The progress of the reaction was monitored by TLC and LCMS. After completion of the reaction, the reaction mixture was concentrated under reduced pressure. The crude residue was washed and titrated with n-hexane (40 mL) to give 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one (700 mg, 1.408 mmol, 97% yield) as an off-white solid. 50 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to give the pure compound (21.86 mg).

### 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one (700 mg, 1.407 mmol) in THF (5 ml) was added 37% formaldehyde (0.7 mL, 7.03 mmol), followed by the addition of STAB (895 mg, 4.22 mmol) portion wise. The reaction mixture was stirred at 25 °C for 2 h. The progress of the reaction was monitored by TLC and LCMS. After completion of the reaction, the reaction mixture was quenched with NH₃ in MeOH followed by TFA at rt. The mixture was diluted with water and extracted with 10% MeOH in DCM (2×100 mL). The combined organic layers was dried over anhydrous Na₂SO₄. Solvent was evaporated under reduced pressure to give the crude compound which was purified by prep HPLC (Column: X-select CSH C18, Mobile Phase A: Ammonium acetate in water, Mobile Phase B: acetonitrile, Flowrate: 15.0 mL/Min, Rt-10.8) to give 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one (96 mg, 0.187 mmol, 13.3% yield) as an off-white solid.

### Example 245. Synthesis of 2-ethyl-6-[(1-methyl-1H-indazol-5-yl)amino]-1-{6-[(1-methylpiperidin-4-yl)oxy]pyridin-2-yl}-1H,2H,3H-pyrazolo[3,4-d]pyrimidine-3-thione (Compound 362)

A stirred suspension of 2-ethyl-6-[(1-methyl-1H-indazol-5-yl)amino]-1-{6-[(1-methylpiperidin-4-yl)oxy]pyridin-2-yl}-1H,2H,3H-pyrazolo[3,4-d]pyrimidin-3-one (100 mg, 0.20 mmol, 1.0 equiv.) and phosphorous pentasulfide (133 mg, 0.30 mmol, 1.5 equiv.) in pyridine (2.5 ml) was heated at 100 °C in a closed vial for 7 h. The resulting yellow solution was diluted with DCM (30 ml) and filtered through a pad of silica, eluted with 1:1 MeOH:EtOAc containing 2.5% NH₃ aq. (conc.), then partitioned between DCM (30 ml) and water (2×30 ml, pH 11), washed with sat. brine and concentrated. The crude material was purified by prep-HPLC, converted to the free base by solid-phase extraction (1 g SCX-2, MeOH, 1.4M NH₃ in MeOH) and concentrated to give the title compound (54 mg, 52%) as a yellow powder.

### Example 246. Synthesis of 2-allyl-1-[6-(1-methyl-4-piperidyloxy)-2-pyridyl]-6-[m-(3-pyridyl)phenylamino]-1,2-dihydro-3H-1,2,5,7-tetraazainden-3-one (Compound 368)

This compound was made using a similar method as described in the synthesis of 2-ethyl-1-[6-(1-methyl-4-piperidyloxy)-2-pyridyl]-6-[m-(5-pyrimidinyl)phenylamino]-1,2-dihydro-3H-1,2,5,7-tetraazainden-3-one. Yield 109.7 mg.

### Example 247. Synthesis of 4-(p-{2-allyl-1-[6-(1-methyl-4-piperidyloxy)-2-pyridyl]-3-oxo-1,2-dihydro-3H-1,2,5,7-tetraazainden-6-ylamino}phenyl)-1λ⁶,4-thiazinane-1,1-dione (Compound 369)

m-chlorobenzeneperoxycarboxylic acid (83.1 mg, 1.2 eq., 481 μmol) was dissolved in dry DCM (2 ml) at RT and added to a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate (0.2 g, 401 μmol) in 6 ml Toluene. The mixture stirred at RT 1 h. DIPEA (210 μL, 3 eq., 1.2 mmol) was added and followed by p-(1,1-dioxo-1λ⁶,4-thiazinan-4-yl)aniline (90.8 mg, 401 μmol). The mixture was stirred at 60 °C and then allowed to cool to RT. The reaction was quenched with 1M NaOH (5 mL) which was added dropwise. The mixture was extracted with ethyl acetate (3×20 mL). The organic phase was washed with brine (20 mL). The combined organic layers were poured through a phase separator and concentrated under reduced pressure to give the product as a yellow powder (180 mg). Deprotection and reductive methylation of the resulting material was performed as described in the synthesis of 2-allyl-6-((4-chlorophenyl)amino)-1-(6-((1-methylpiperidin-4-yl)amino)pyridin-2-yl)-1,2-dihydro-3H-pyrazolo[3,4-d]pyrimidin-3-one. Yield 141 mg.

### Example 248. Synthesis of 2-allyl-1-(2-((1-(oxetan-3-yl)piperidin-4-yl)oxy)pyrimidin-4-yl)-6-((4-(2,2,2-trifluoroethoxy)phenyl)amino)-1,2-dihydro-3H-pyrazolo[3,4-d]pyrimidin-3-one (Compound 371)

To a solution of 2-allyl-1-(2-(piperidin-4-yloxy)pyrimidin-4-yl)-6-((4-(2,2,2-trifluoroethoxy)phenyl)amino)-1,2-dihydro-3H-pyrazolo[3,4-d]pyrimidin-3-one (400 mg, 0.737 mmol) in 1,2-Dichloroethane (10 ml) was added oxetan-3-one (106 mg, 1.475 mmol) and stirred at 60 °C for 1 h. The mixture was cooled to rt and STAB (469 mg, 2.212 mmol) was added. The resulting reaction mixture was stirred for 3 h. The progress of the reaction was monitored by TLC and LCMS. After completion of the reaction, the reaction mixture was quenched with ice cold water (100 mL) and extracted with 10% MeOH-DCM (2×100 mL). The combined organic layers was dried over anhydrous Na₂SO₄, evaporated under reduced pressure to give the crude compound which was purified by Prep HPLC (X Select C-8, 19×250, Mobile phase A: 10 mm ABC in Water, Mobile phase B: acetonitrile) to give 2-allyl-1-(2-((1-(oxetan-3-yl)piperidin-4-yl)oxy)pyrimidin-4-yl)-6-((4-(2,2,2-trifluoroethoxy)phenyl)amino)-1,2-dihydro-3H-pyrazolo[3,4-d]pyrimidin-3-one (73 mg, 0.112 mmol, 15.14% yield) as an off-white solid.

### Example 249. Synthesis of 2-allyl-6-((1-isopropyl-1H-indazol-5-yl)amino)-1-(2-(piperidin-4-yloxy)pyrimidin-4-yl)-1,2-dihydro-3H-pyrazolo[3,4-d]pyrimidin-3-one (Compound 375) and 2-allyl-6-((1-isopropyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one (Compound 401)

### 1-isopropyl-5-nitro-1H-indazole

To a stirred solution of 5-nitro-1H-indazole (1 g, 6.13 mmol) in DMF (10 ml), was added DBU (1.109 ml, 7.36 mmol) and 2-iodopropane (0.736 ml, 7.36 mmol) at 0 °C. The resulting reaction mixture was stirred at rt for 3 h. The progress of the reaction was monitored by TLC and LCMS. After completion of the reaction, the reaction mixture was quenched with water (100 mL) and extracted with ethyl acetate (2×100 mL) to give crude material which was purified by column chromatography (SiO₂/230-400 mesh; 30-50% ethyl acetate in hexane) to give 1-isopropyl-5-nitro-1H-indazole (700 mg, 3.41 mmol, 55.6% yield) as an off-white solid. This compound structure was also confirmed by 1D NOE.

### 1-isopropyl-1H-indazol-5-amine

To a degassed solution of 1-isopropyl-5-nitro-1H-indazole (600 mg, 2.92 mmol) in methanol (20 ml) was added 10% Pd/C (311 mg, 0.292 mmol) under nitrogen atmosphere. The reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through celite and washed with methanol (2×100 mL). The filtrate was concentrated under reduced pressure to give 1-isopropyl-1H-indazol-5-amine (500 mg, 2.85 mmol, 98% yield) as a black gummy solid, which was taken into the next step without further purification.

### tert-butyl 4-((4-(2-allyl-6-((1-isopropyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (500 mg, 0.941 mmol) in AcOH (10 ml) was added 1-isopropyl-1H-indazol-5-amine (247 mg, 1.411 mmol) at room temperature. The reaction mixture was stirred at 25 °C for 16 h. The progress of the reaction was monitored by TLC and LCMS. After completion of the reaction, the solvent was evaporated under reduced pressure, and then the reaction mixture was quenched with aq. NaHCO₃ (50 mL) at 0 °C and extracted with 10% methanol in DCM (2×50 mL). The combined organic layers was dried over Na₂SO₄, filtered, and concentrated under reduced pressure to give tert-butyl 4-((4-(2-allyl-6-((1-isopropyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate (500 mg, 0.798 mmol, 85% yield) as a brown gummy solid.

### 2-allyl-6-((1-isopropyl-1H-indazol-5-yl)amino)-1-(2-(piperidin-4-yloxy)pyrimidin-4-yl)-1,2-dihydro-3H-pyrazolo[3,4-d]pyrimidin-3-one

To a stirred solution of tert-butyl 4-((4-(2-allyl-6-((1-isopropyl-1H-indazol-5-yl)amino)-3-oxo-2,3-dihydro-1H-pyrazolo[3,4-d]pyrimidin-1-yl)pyrimidin-2-yl)oxy)piperidine-1-carboxylate (600 mg, 0.798 mmol) in DCM (15 ml) was added TFA (0.916 ml, 11.97 mmol) at 0 °C under inert atmosphere. Then, the resulting reaction mixture was stirred at room temperature for 16 h. The progress of the reaction was monitored by TLC and LCMS. After completion of the reaction, the solvent was evaporated under reduced pressure, and the solid was collected. This solid was washed with n-hexane to give the compound 2-allyl-6-((1-isopropyl-1H-indazol-5-yl)amino)-1-(2-(piperidin-4-yloxy)pyrimidin-4-yl)-1,2-dihydro-3H-pyrazolo[3,4-d]pyrimidin-3-one (400 mg, 0.471 mmol, 59.0% yield) as a brown solid. 50 mg of the above compound was taken and purified by Prep HPLC (X Select C18, 19×250), Mobile phase A: 0.1% FA in H2O, Mobile phase B: acetonitrile) to give the pure compound (22.23 mg).

### 2-allyl-6-((1-isopropyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one

To a stirred solution of 2-allyl-6-((1-isopropyl-1H-indazol-5-yl)amino)-1-(2-(piperidin-4-yloxy)pyrimidin-4-yl)-1,2-dihydro-3H-pyrazolo[3,4-d]pyrimidin-3-one (300 mg, 0.570 mmol) in THF (5 ml) was added 37% formaldehyde (0.240 ml, 1.946 mmol), followed by the addition of STAB (362 mg, 1.709 mmol) at rt. The mixture was stirred for 2 h. The progress of the reaction was monitored by TLC and LCMS. After completion of the reaction, the reaction mixture was quenched with NH₃ in MeOH followed by TFA at rt and water. The mixture was extracted with 10% MeOH in DCM (2×100 mL). The combined organic layers was dried over anhydrous Na₂SO₄ and evaporated under reduced pressure to give the crude compound which was purified by prep HPLC (X Select C18, 19×250), Mobile phase A: 0.1% FA in H₂O, Mobile phase B: acetonitrile) to give the pure compound 2-allyl-6-((1-isopropyl-1H-indazol-5-yl)amino)-1-(2-((1-methylpiperidin-4-yl)oxy)pyrimidin-4-yl)-1,2-dihydro-3H-pyrazolo[3,4-d]pyrimidin-3-one (15 mg, 0.027 mmol, 4.73% yield) as off white solid.

### Example 250. Synthesis of 2-allyl-6-((1-cyclopropyl-1H-indazol-5-yl)amino)-1-(6-(piperidin-4-yloxy)pyridin-2-yl)-1,2-dihydro-3H-pyrazolo[3,4-d]pyrimidin-3-one (Compound 376) and 2-allyl-6-((1-cyclopropyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 386)

### 1-cyclopropyl-5-nitro-1H-indazole

To a stirred solution of 5-nitro-1H-indazole (1.5 g, 9.19 mmol) in DCE (50 ml) were added sodium carbonate (1.949 g, 18.39 mmol), cyclopropylboronic acid (1.580 g, 18.39 mmol), 2-(2-pyridyl)pyridine (1.436 g, 9.19 mmol) and copper (II) acetate (1.670 g, 9.19 mmol). The resulting reaction mixture was stirred at 70 °C for 3 h. The progress of the reaction was monitored by TLC and LCMS. After completion of the reaction, the reaction mixture was quenched with water (100 mL) and extracted with ethyl acetate (2×100 mL). The combined organic layers were dried over anhydrous Na₂SO₄, filtered, and evaporated under reduced pressure to give crude compound which was purified by column chromatography (SiO₂/100-200 mesh; 40-50% ethyl acetate-hexane) to give 1-cyclopropyl-5-nitro-1H-indazole (3, 700 mg, 3.44 mmol, 37.5% yield).

### 1-cyclopropyl-1H-indazol-5-amine

To a stirred solution of 1-cyclopropyl-5-nitro-1H-indazole (1 g, 4.92 mmol) in methanol (20 ml) was added 10% Pd/C (0.524 g, 0.492 mmol) under nitrogen atmosphere. The reaction mixture was stirred under H₂ bladder pressure at room temperature for 16 h. The progress of the reaction was monitored by TLC and UPLC. After completion of the reaction, the reaction mixture was filtered through a pad of celite and washed with methanol (2×100 mL). The filtrate was concentrated under reduced pressure to give 1-cyclopropyl-1H-indazol-5-amine (700 mg, 4.04 mmol, 82% yield) as a black gummy solid, which was taken into the next step without further purification.

### tert-butyl 4-((6-(2-allyl-6-((1-cyclopropyl-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (500 mg, 0.942 mmol) in AcOH (10 ml) was added 1-cyclopropyl-1H-indazol-5-amine (163 mg, 0.942 mmol) at room temperature. The reaction mixture was stirred at 25 °C for 16 h. The progress of the reaction was monitored by TLC and LCMS. After completion of the reaction, the solvent was evaporated under reduced pressure and the residue was quenched with aq. NaHCO₃ (100 mL) at 0 °C and extracted with 10% MeOH-DCM (2×50 mL). The combined organic layers was dried over Na₂SO₄, filtered, and concentrated under reduced pressure to give tert-butyl 4-((6-(2-allyl-6-((1-cyclopropyl-1H-indazol-5-yl)amino)-3-oxo-2,3-dihydro-1H-pyrazolo[3,4-d]pyrimidin-1-yl)pyridin-2-yl)oxy)piperidine-1-carboxylate (500 mg, 0.802 mmol, 85% yield) as a brown gummy solid.

### 2-allyl-6-((1-cyclopropyl-1H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-cyclopropyl-1H-indazol-5-yl)amino)-3-oxo-2,3-dihydro-1H-pyrazolo[3,4-d]pyrimidin-1-yl)pyridin-2-yl)oxy)piperidine-1-carboxylate (700 mg, 1.122 mmol) in DCM (15 ml) was added TFA (0.7 ml, 8.98 mmol) at 0 °C under inert atmosphere. The resulting reaction mixture was stirred at room temperature for 16 h. The progress of the reaction was monitored by TLC and LCMS. After completion of the reaction, the reaction mixture was concentrated under reduced pressure. The crude residue was triturated with n-hexane (10 mL) to give 2-allyl-6-((1-cyclopropyl-1H-indazol-5-yl)amino)-1-(6-(piperidin-4-yloxy)pyridin-2-yl)-1,2-dihydro-3H-pyrazolo[3,4-d]pyrimidin-3-one (500 mg, 0.955 mmol, 85% yield) as an off-white solid. 50 mg of the above compound was taken and purified by prep HPLC (Column: X-select CSH C18, Mobile Phase A: 0.1% FA in H₂O, Mobile Phase B: acetonitrile, Flowrate: 15.0 mL/Min, Rt-10.8) to give the pure compound (6.87 mg).

### 2-allyl-6-((1-cyclopropyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-cyclopropyl-1H-indazol-5-yl)amino)-1-(6-(piperidin-4-yloxy)pyridin-2-yl)-1,2-dihydro-3H-pyrazolo[3,4-d]pyrimidin-3-one (350 mg, 0.668 mmol) in THF (5 ml) was added 37% formaldehyde (0.184 ml, 6.68 mmol), followed by the addition of STAB (425 mg, 2.005 mmol) at 0 °C. The resulting reaction mixture was stirred at room temperature for 1 h. The progress of the reaction was monitored by TLC and LCMS. After completion of the reaction, the reaction mixture was quenched with NH₃ in MeOH followed by TFA at rt. The mixture was diluted with water and extracted with 10% MeOH in DCM (2×100 mL). The combined organic layers were dried over anhydrous Na₂SO₄ and evaporated under reduced pressure to give the crude compound, which was purified by prep HPLC (Shim pack C18, 250 mm×20, Mobile phase A: 10 mm AA in Water, Mobile phase B: acetonitrile, Flow: 15 mL) to give 2-allyl-6-((1-cyclopropyl-1H-indazol-5-yl)amino)-1-(6-((1-methylpiperidin-4-yl)oxy)pyridin-2-yl)-1,2-dihydro-3H-pyrazolo[3,4-d]pyrimidin-3-one (100 mg, 0.186 mmol, 27.8% yield) as an off-white solid.

### Example 251. Synthesis of 2-allyl-6-((4-(3-fluoropropoxy)phenyl)amino)-1-(6-((1-methylpiperidin-4-yl)oxy)pyridin-2-yl)-1,2-dihydro-3H-pyrazolo[3,4-d]pyrimidin-3-one (Compound 377) and 2-allyl-6-((4-(3-fluoropropoxy)phenyl)amino)-1-(6-(piperidin-4-yloxy)pyridin-2-yl)-1,2-dihydro-3H-pyrazolo[3,4-d]pyrimidin-3-one (Compound 378)

### (1-(3-fluoropropoxy)-4-nitrobenzene

To a stirred solution of 4-nitrophenol (1 g, 7.19 mmol) in DMF (10 mL) was added potassium carbonate (0.993 g, 7.19 mmol) at 0 °C and the mixture was stirred for 1 h at room temperature. Then 1-fluoro-3-iodopropane (1.061 ml, 10.78 mmol) was added to the reaction mixture under an argon atmosphere. The resulting reaction mixture was stirred at 25 °C for 16 h. The progress of the reaction was monitored by TLC. After completion of the reaction, the reaction mixture was concentrated under reduced pressure. The resulting crude material was purified by flash chromatography (SiO₂/230-400 mesh; ~50-80% EtOAc/hexane) to afford 1-(3-fluoropropoxy)-4-nitrobenzene (1.5 g, 5.50 mmol, 76% yield) as a yellow solid.

### 4-(3-fluoropropoxy)aniline

To a degassed solution of 1-(3-fluoropropoxy)-4-nitrobenzene (1.5 g, 7.53 mmol) in MeOH (20 ml) was added Pd—C (10%, 217 mg, 2.039 mmol) under inert atmosphere. The reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure to give 4-(3-fluoropropoxy)aniline (630 mg, 3.7 mmol) as a brown gummy solid, which was taken into the next step without further purification.

### tert-butyl 4-((6-(2-allyl-6-((4-(3-fluoropropoxy)phenyl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (600 mg, 1.131 mmol) in acetonitrile (5 mL) was added 4-(3-fluoropropoxy)aniline (210 mg, 1.244 mmol) at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using methanol-DCM (15-20%) as an eluent to obtain tert-butyl 4-((6-(2-allyl-6-((4-(3-fluoropropoxy)phenyl)amino)-3-oxo-2,3-dihydro-1H-pyrazolo[3,4-d]pyrimidin-1-yl)pyridin-2-yl)oxy)piperidine-1-carboxylate (510 mg, 0.757 mmol, 67.0% yield) as a brown solid.

### 2-allyl-6-((4-(3-fluoropropoxy)phenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((4-(3-fluoropropoxy)phenyl)amino)-3-oxo-2,3-dihydro-1H-pyrazolo[3,4-d]pyrimidin-1-yl)pyridin-2-yl)oxy)piperidine-1-carboxylate (510 mg, 0.823 mmol) in 1,4-dioxane (5 mL), was added 4M HCl in 1,4-dioxane (2.0 mL) at 0 °C. The reaction mixture was stirred at room temperature for 4 h. The progress of the reaction was monitored by TLC. After completion of the reaction, the reaction mixture was concentrated under reduced pressure. The crude residue (70 mg) was purified by prep. HPLC X-Select C18 (19*250, 5 mL), Mobile phase A: 0.1 M FA in H₂O, Mobile phase B: acetonitrile) to get 2-allyl-6-((4-(3-fluoropropoxy)phenyl)amino)-1-(6-(piperidin-4-yloxy)pyridin-2-yl)-1,2-dihydro-3H-pyrazolo[3,4-d]pyrimidin-3-one (70 mg, 0.111 mmol, 72% Yield) as an off white-solid.

### 2-allyl-6-((4-(3-fluoropropoxy)phenyl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((4-(3-fluoropropoxy)phenyl)amino)-1-(6-(piperidin-4-yloxy)pyridin-2-yl)-1,2-dihydro-3H-pyrazolo[3,4-d]pyrimidin-3-one (400 mg, 0.770 mmol) in THF (10 mL) was added 20% aq. formaldehyde (8 ml) at 25 °C. The reaction mixture was stirred at 25 °C for 5 min. Then, STAB (575 mg, 2.71 mmol) was added portion-wise. After the complete addition of STAB, the reaction mixture was stirred at 25 °C for 20 min. The progress of the reaction was monitored by TLC and LCMS. After completion of the reaction, the reaction mixture was quenched with TFA, followed by NH₃ in MeOH, diluted with water and extracted with 10% MeOH in DCM (2×100 mL). The combined organic extracts were washed with NaHCO₃, dried over anhydrous Na₂SO₄, filtered, and concentrated under reduced pressure to afford crude compound that was purified by prep. HPLC ZORBAX (30 MM), Mobile phase A: 10 MM ABC in H₂O, Mobile phase B: acetonitrile) to give 2-allyl-6-((4-(3-fluoropropoxy)phenyl)amino)-1-(6-((1-methylpiperidin-4-yl)oxy)pyridin-2-yl)-1,2-dihydro-3H-pyrazolo[3,4-d]pyrimidin-3-one (71.57 mg, 0.133 mmol, 17.25% yield) as an off-white solid.

### Example 252. Synthesis of rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (Compound 404); rel-2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (Compound 402); rel-2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one (Compound 405); rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (Compound 403); 2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one (Compound 379); rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (Compound 380); 2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (Compound 381); and 2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (Compound 382)

### tert-butyl 4-hydroxy-2-methylpiperidine-1-carboxylate

To a stirred solution of tert-butyl 2-methyl-4-oxopiperidine-1-carboxylate (3 g, 14.07 mmol) in MeOH (50 mL) was added sodium borohydride (1.064 g, 28.1 mmol) at 0 °C, the reaction was allowed to stir at 25 °C for 16 h. The progress of the reaction was monitored by TLC. After completion of the reaction, the reaction mixture was diluted with water and extracted with ethyl acetate (100 mL×2). The combined organic layer was dried over Na₂SO₄, filtered, and concentrated under reduced pressure to afford tert-butyl 4-hydroxy-2-methylpiperidine-1-carboxylate (3 g, 13.93 mmol) as a colourless liquid.

### tert-butyl 4-((6-bromopyridin-2-yl)oxy)-2-methylpiperidine-1-carboxylate

To a stirred solution of tert-butyl 4-hydroxy-2-methylpiperidine-1-carboxylate (3 g, 13.93 mmol) in THF (10 ml) was added sodium hydride (60% in oil, 0.669 g, 16.72 mmol). After 10 min at 25 °C, was added 2,6-dibromopyridine (3.14 g, 13.24 mmol) at 0 °C. The reaction was allowed to stir at 25 °C for 2 h. The progress of the reaction was monitored by TLC and UPLC. After completion of the reaction, ice water was added, and the product was extracted with ethyl acetate. The combined organic layer was washed with brine, dried over anhydrous sodium sulfate, and concentrated under reduced pressure. The resulting crude material was purified by flash chromatography (SiO₂/100-200 mesh; 10-20% ethyl acetate/hexane) to afford tert-butyl 4-((6-bromopyridin-2-yl)oxy)-2-methylpiperidine-1-carboxylate (4 g, 9.37 mmol, 67.3% yield) as an off-white solid.

### tert-butyl 4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate

A stirred solution of tert-butyl 4-((6-bromopyridin-2-yl)oxy)-2-methylpiperidine-1-carboxylate (2 g, 5.39 mmol), 2-allyl-6-(methylthio)-1,2-dihydro-3H-pyrazolo[3,4-d]pyrimidin-3-one (1.437 g, 6.46 mmol), potassium carbonate (2.233 g, 16.16 mmol) and N,N′-dimethylethane-1,2-diamine (0.475 g, 5.39 mmol) in dioxane (30 ml) was degassed for 20 min at rt under inert atmosphere. Then was added copper (I) iodide (1.231 g, 6.46 mmol) and the mixture was stirred at 100 °C for 16 h. The progress of the reaction was monitored by UPLC. After completion of the reaction, the reaction mass was diluted with DCM and filtered through celite pad. The collected filtrate was concentrated under reduced pressure. The crude material was purified by flash column chromatography (silica gel, 100-200 mesh size) using ethyl acetate-petroleum ether (10%-20%) as an eluent to obtain tert-butyl 4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (2.2 g, 4.25 mmol, 79% yield) off white solid.

### tert-butyl (4S)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate; tert-butyl(2S,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate; and tert-butyl(2R,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate

The tert-butyl (4S)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (6, 6.6 g) enantiomers were separated by chiral SFC (I-CELLULOSE-C_0.5% IPAm in IPA_30). Fractions were concentrated and lyophilized to give:
tert-butyl (4S)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (1.65 g) as an off white solid;
ter-butyl(2S,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (1 g) as an off white solid; and
tert-butyl(2R,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (1.4 g) as an off white solid.

### tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate

To a stirred solution of tert-butyl 4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (400 mg, 0.780 mmol) in DCM (10 ml), mCPBA (65-70%, 148 mg, 0.858 mmol) was added at 0 °C and the mixture was stirred at RT for 2 h. The progress of the reaction was monitored by TLC and UPLC. After completion of the reaction, the reaction mixture was quenched with saturated sodium thiosulphate pentahydrate (100 mL) and extracted with DCM (250 mL×2). The combined organic extract was washed with saturated aqueous sodium bicarbonate solution (100 mL). The organic extract obtained was dried over anhydrous sodium sulfate, filtered, and concentrated under reduced pressure (bath temperature: 45 °C) to afford crude tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (400 mg, 0.643 mmol, 82% yield) as sticky solid.

### tert-butyl 4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (1.65 g, 3.12 mmol) in acetonitrile (15 mL) was added 1-methyl-1H-indazol-5-amine (0.551 g, 3.75 mmol) at room temperature. The reaction mixture was subjected to stirring at 70 °C for 16 h.

The progress of the reaction was monitored by TLC. After the completion of the reaction, the reaction mixture was concentrated under reduced pressure. The crude product (1.8 g) was purified by achiral SFC purification. Fractions were concentrated and lyophilized to obtain tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.1 g, 1.798 mmol, 57.6% yield) as a reddish brown solid.

### tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate and tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate

The tert-butyl (4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.1 g, 1.798 mmol) was purified by chiral SFC (Lux-i-Amylose-3, 0.5% IPAm in IPA_ACN) to obtain:
tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (350 mg, 0.566 mmol, 31.5% yield) as a yellow solid; and
tert-butyl(2S,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (360 mg, 0.559 mmol, 31.1% yield) as a brown solid.
rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one

To a stirred solution of tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (360 mg, 0.589 mmol) in 1,4-dioxane (10 mL), was added 4M HCl in 1,4-dioxane (5.0 mL) at 0 °C. The resulting reaction mixture was stirred at room temperature for 4 h. The progress of the reaction was monitored by TLC. After the completion of the reaction, the reaction mixture was concentrated under reduced pressure. The crude material was washed with hexane and MTBE. The residue obtained was then dried under vacuum to give rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (290 mg, 0.561 mmol, 95% yield) as a yellow solid (82.1 mg).

### rel-2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one

To a stirred solution of rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (250 mg, 0.782 mmol) in THF (10 mL) was added formaldehyde (37% aq., 5 mL) at 25 °C. The reaction mixture was stirred at 25 °C for 5 min. Then sodium triacetoxyborohydride (331 mg, 1.564 mmol) was added portion-wise. After the complete addition of STAB, the reaction mixture was stirred at 25 °C for 20 min. The progress of the reaction was monitored by TLC and LCMS. The reaction mixture was quenched with TFA, followed by NH₃ in MeOH, then diluted with water and extracted with 10% MeOH in DCM (2×100 mL). The combined organic extract was washed with NaHCO₃, dried over anhydrous Na₂SO₄, filtered, and concentrated under reduced pressure to afford crude compound which was purified by prep. HPLC X-Select C18 (19*250, 5 mL), Mobile phase A: 10 MM AA in H₂O, Mobile phase B: acetonitrile) to give rel-2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (139.5 mg, 0.263 mmol, 33.6% yield) as a white fluffy solid.

### rel-2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one

To a stirred solution of tert-butyl(2S,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (400 mg, 0.572 mmol) in 1,4-dioxane (10 mL), was added 4M HCl in 1,4-dioxane (5.0 mL) at 0 °C. The reaction mixture was stirred at room temperature for 4 h. The progress of the reaction was monitored by TLC. After the completion of the reaction, the reaction mixture was concentrated under reduced pressure. The crude material was washed with hexane and MTBE. The residue was then dried under vacuum to obtain rel-2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one (320 mg, 0.620 mmol, 99% yield) as a reddish brown solid.

### rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one

To a stirred solution of rel-2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one (300 mg, 0.586 mmol) in THF (10 mL) was added formaldehyde (37% aq., 6 mL) at 25 °C). The reaction mixture was stirred at 25 °C for 5 min. sodium triacetoxyborohydride (249 mg, 1.173 mmol) was added portion-wise. After addition, the reaction mixture was stirred at 25 °C for 20 min. The progress of the reaction was monitored by TLC and LCMS. The reaction mixture was quenched with TFA followed by NH₃ in MeOH, then diluted with water and extracted with 10% MeOH in DCM (2×100 mL). The combined organic extract was washed with NaHCO₃, dried over anhydrous Na₂SO₄, filtered, and concentrated under reduced pressure to afford crude compound which was purified by prep. HPLC X-Select C18 (19*250, 5 mL), Mobile phase A: 10 MM AA in H₂O, Mobile phase B: acetonitrile) to give rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (163.2 mg, 0.309 mmol, 52.7% yield) as a white fluffy solid.

### tert-butyl(2S,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate

To a stirred solution of tert-butyl(2R,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (1.4 g, 2.73 mmol) in DCM (50 ml) at 0 °C, was added mCPBA (65-70%, 262 mg, 1.519 mmol) and the mixture was stirred at RT for 2 h. The progress of the reaction was monitored by TLC and UPLC. After completion of the reaction, ice water was added and the mixture was extracted with DCM (100 mL×2). The combined organic layers was washed with aqueous sodium bicarbonate solution, dried over anhydrous sodium sulfate, and concentrated under reduced pressure to obtain tert-butyl(2R,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (1.40 g, 2.03 mmol, 93% yield) as a pale yellow semi-solid.

### rel-tert-butyl(2R,4S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate

To a stirred solution rel-tert-butyl(2R,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (1.4 g, 2.65 mmol) in acetonitrile (30 mL) was added 1-methyl-1H-indazol-5-amine (0.429 g, 2.91 mmol) at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using ethyl acetate-hexane (40% to 50%) as an eluent to obtain rel-tert-butyl(2S,4S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.03 g, 1.566 mmol, 59.1% yield) as a brown solid.

### 2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one

To a stirred solution of rel-tert-butyl(2S,4S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.3 g, 2.125 mmol) in 1,4-dioxane (5 mL), was added 4M HCl in 1,4-dioxane (2.0 mL) at 0 °C. The resulting reaction mixture was stirred at room temperature for 4 h. The progress of the reaction was monitored by TLC. After completion of the reaction, the reaction mixture was concentrated under reduced pressure to give crude product 1100 mg). The crude residue (450 mg) was purified by prep. HPLC X-Select C18 (19*250, 5 mL), Mobile phase A: 10 MM AA in H₂O, Mobile phase B: acetonitrile) to give rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (250 mg, 0.192 mmol, 67% yield) as an off white-solid.

### rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one

To a stirred solution of rel-2-allyl-6-((1-methyl-1H-indazol-5-yl)amino)-1-(6-(((2R,4S)-2-methylpiperidin-4-yl)oxy)pyridin-2-yl)-1,2-dihydro-3H-pyrazolo[3,4-d]pyrimidin-3-one (500 mg, 0.977 mmol) in THF (10 mL) was added formaldehyde (37% aq., 5 mL, 2.93 mmol) at 25 °C. The reaction mixture was stirred at 25 °C for 5 min. sodium triacetoxyborohydride (621 mg, 2.93 mmol) was added portion-wise. The reaction mixture was stirred at 25 °C for 20 min. The progress of the reaction was monitored by TLC and LCMS. The reaction mixture was quenched with TFA, followed by NH₃ in MeOH, diluted with water and extracted with 10% MeOH in DCM (2×100 mL). The combined organic extracts was washed with NaHCO₃, dried over anhydrous Na₂SO₄, filtered, and concentrated under reduced pressure to afford crude compound which was purified by prep. HPLC X-Select C18 (19*250, 5 mL), Mobile phase A: 10 MM AA in H₂O, Mobile phase B: acetonitrile) to give rel-2-allyl-1-(6-(((2R,4S)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (265.5 mg, 0.504 mmol, 52% yield) as an off-white solid.

### tert-butyl(2R,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate

To a stirred solution of tert-butyl(2R,4R)-4-((6-(2-allyl-6-(methylthio)-3-oxo-2,3-dihydro-1H-pyrazolo[3,4-d]pyrimidin-1-yl)pyridin-2-yl)oxy)-2-methylpiperidine-1-carboxylate (1.4 g, 2.73 mmol) in DCM (5 ml) at 0 °C, was added m-CPBA (70%, 0.518 g, 3.00 mmol) and the mixture was stirred at RT for 2 h. The progress of the reaction was monitored by TLC and UPLC. After completion of the reaction, the reaction mixture was added to ice water and extracted with DCM (100 mL×2). The combined organic layer was washed with aqueous sodium bicarbonate solution, dried over anhydrous sodium sulfate and concentrated under reduced pressure to obtain tert-butyl(2R,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (1.4 g, 2.65 mmol) as a pale yellow semi-solid.

### tert-butyl(2R,4R)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate

To a stirred solution of tert-butyl(2R,4R)-4-((6-(2-allyl-6-(methylsulfinyl)-3-oxo-2,3-dihydro-1H-pyrazolo[3,4-d]pyrimidin-1-yl)pyridin-2-yl)oxy)-2-methylpiperidine-1-carboxylate (1.4 g, 2.65 mmol) in acetonitrile (20 mL) was added 1-methyl-1H-indazol-5-amine (0.429 g, 2.91 mmol) at room temperature. The reaction mixture was stirred at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using ethyl acetate-hexane (40% to 50%) as an eluent to obtain tert-butyl(2S,4S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.03 g, 1.566 mmol, 59.1% yield) as a gummy oil.

### 2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one

To a stirred solution of tert-butyl(2S,4S)-4-((6-(2-allyl-6-((1-methyl-1H-indazol-5-yl)amino)-3-oxo-2,3-dihydro-1H-pyrazolo[3,4-d]pyrimidin-1-yl)pyridin-2-yl)oxy)-2-methylpiperidine-1-carboxylate (1.3 g, 2.125 mmol) in 1,4-dioxane (5 mL), was added 4M HCl in 1,4-dioxane (2.0 mL) at 0 °C. The resulting reaction mixture was stirred at room temperature for 4 h. The reaction was monitored by TLC. After completion of the reaction, the reaction mixture was concentrated under reduced pressure to afford the crude product (1100 mg). The crude residue (450 mg) was purified by prep. HPLC X-Select C18 (19*250, 5 mL), Mobile phase A: 10 MM AA in H₂O, Mobile phase B: acetonitrile) to give rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (234 mg, 0.364 mmol, 72% yield) as an off white-solid.

### 2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one

To a stirred solution of rel-2-allyl-6-((1-methyl-1H-indazol-5-yl)amino)-1-(6-(((2R,4R)-2-methylpiperidin-4-yl)oxy)pyridin-2-yl)-1,2-dihydro-3H-pyrazolo[3,4-d]pyrimidin-3-one (650 mg, 1.955 mmol) in THF (10 mL) was added formaldehyde (37% aq., 10 mL) at 25 °C. The reaction mixture was stirred at 25 °C for 5 min. Then was added, sodium triacetoxyborohydride (1.243 g, 5.86 mmol) portion-wise. After the complete addition, the reaction mixture was stirred at 25 °C for 20 min. The progress of the reaction was monitored by TLC and LCMS. After completion of the reaction, the reaction mixture was quenched with TFA, followed by NH₃ in MeOH, diluted with water and extracted with 10% MeOH in DCM (2×100 mL). The combined organic extracts were washed with NaHCO₃, dried over anhydrous Na₂SO₄, filtered, and concentrated under reduced pressure to afford crude compound that was purified by prep. HPLC X-Select C18 (19*250, 5 mL), Mobile phase A: 10 MM AA in H₂O, Mobile phase B: acetonitrile) to give rel-2-allyl-1-(6-(((2R,4R)-1,2-dimethylpiperidin-4-yl)oxy)pyridin-2-yl)-6-((1-methyl-1H-indazol-5-yl)amino)-1,2-dihydro-3H-pyrazolo[3,4-d]pyrimidin-3-one (200 mg, 0.380 mmol, 20.1% yield) as an off-white solid.

### Example 253. Synthesis of Synthesis of 2-allyl-6-((1-(3-fluoropropyl)-1H-indazol-5-yl)amino)-1-(6-(piperidin-4-yloxy)pyridin-2-yl)-1,2-dihydro-3H-pyrazolo[3,4-d]pyrimidin-3-one (Compound 397) and 2-allyl-6-((1-(3-fluoropropyl)-1H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 387)

### 1-(3-fluoropropyl)-5-nitro-1H-indazole

To a stirred solution of 5-nitro-1H-indazole (1.0 g, 6.13 mmol) in DMF (10 mL) was added NaH (0.294 g, 7.36 mmol) at 0 °C and the mixture was stirred for 1 h at room temperature. Then 1-fluoro-3-iodopropane (0.738 ml, 7.36 mmol) was added to the reaction mixture under argon atmosphere. The resulting reaction mixture was stirred at 90 °C for 16 h. The progress of reaction was monitored by TLC. After completion of the reaction, the reaction mixture was diluted with cold water and the precipitated solid was filtered and dried. The crude residue was purified by prep. HPLC (Shimpak C18 (250*19.5 um) Mobile phase A: 10 MM ABC in H₂O, Mobile phase B: acetonitrile) to give 1-(3-fluoropropyl)-5-nitro-1H-indazole (0.300 g, 1.344 mmol, 21.93% yield) as a yellow solid.

### 1-(3-fluoropropyl)-1H-indazol-5-amine

To a degassed solution of 1-(3-fluoropropyl)-5-nitro-1H-indazole (0.300 g, 1.344 mmol) in MeOH (10 ml) was added Pd—C (100 mg, 2.039 mmol) under inert atmosphere. Then the reaction mixture was stirred under hydrogen atmosphere using hydrogen bladder pressure at room temperature for 16 h. The progress of reaction was monitored by TLC and UPLC. After completion of the reaction, the reaction mixture was filtered under nitrogen atmosphere through a pad of celite and washed with MeOH (2×100 mL). The filtrate was concentrated under reduced pressure to give 1-(3-fluoropropyl)-1H-indazol-5-amine (0.250 g, 1.100 mmol, 82% yield) as a black gummy solid, which was taken into the next step without further purification.

### tert-butyl 4-((6-(2-allyl-6-((1-(3-fluoropropyl)-1H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (0.500 g, 0.972 mmol) in acetonitrile (5 mL) was added 1-(3-fluoropropyl)-1H-indazol-5-amine (0.225 g, 1.166 mmol) at room temperature. The reaction mixture was subjected to stirring at 70 °C for 16 h. The progress of reaction was monitored by TLC. The reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using methanol-DCM (15-20%) as an eluent to obtain tert-butyl 4-((6-(2-allyl-6-((1-(3-fluoropropyl)-1H-indazol-5-yl)amino)-3-oxo-2,3-dihydro-1H-pyrazolo[3,4-d]pyrimidin-1-yl)pyridin-2-yl)oxy)piperidine-1-carboxylate (0.3 g, 0.47 mmol, 48% yield) as a brown solid.

### 2-allyl-6-((4-(3-fluoropropoxy)phenyl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-(3-fluoropropyl)-1H-indazol-5-yl)amino)-3-oxo-2,3-dihydro-1H-pyrazolo[3,4-d]pyrimidin-1-yl)pyridin-2-yl)oxy)piperidine-1-carboxylate (0.300 g, 0.466 mmol) in 1,4-dioxane (5 mL), was added 4N HCl in 1,4-dioxane (2 mL) at 0 °C. The resulting reaction mixture was stirred at room temperature for 4 h. The progress of reaction was monitored by TLC. After completion of the reaction, the reaction mixture was concentrated under reduced pressure. The crude residue was purified by prep. HPLC (Shimpak C18 (250*19.5 um) Mobile phase A: 10 MM ABC in H₂O, Mobile phase B: acetonitrile) to give 2-allyl-6-((1-(3-fluoropropyl)-1H-indazol-5-yl)amino)-1-(6-(piperidin-4-yloxy)pyridin-2-yl)-1,2-dihydro-3H-pyrazolo[3,4-d]pyrimidin-3-one (14 mg, 0.024 mmol, 5.5% yield) as an off white-solid.

### 2-allyl-6-((1-(3-fluoropropyl)-1H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-(3-fluoropropyl)-1H-indazol-5-yl)amino)-1-(6-(piperidin-4-yloxy)pyridin-2-yl)-1,2-dihydro-3H-pyrazolo[3,4-d]pyrimidin-3-one (0.300 g, 0.552 mmol) in THF (10 mL) was added formaldehyde (6 mL) at 25 °C, then reaction mixture stirred at 25 °C for 5 min. Then was added STAB (0.234 g, 1.104 mmol) portion wise. After complete addition of STAB, the reaction mixture was stirred at 25 °C for 20 mins. The progress of the reaction was monitored by TLC and LCMS. The reaction mixture was quenched with TFA followed by NH₃ in MeOH and diluted with water and extracted with 10% MeOH in DCM (2×100 mL). The combined organic extracts was washed with NaHCO₃, dried over anhydrous Na₂SO₄, filtered and concentrated under reduced pressure to afford crude compound which was purified by prep. HPLC (Shimpak C18 (250*19.5 um) Mobile phase A: 10 MM ABC in H₂O, Mobile phase B: acetonitrile) to give 2-allyl-6-((1-(3-fluoropropyl)-1H-indazol-5-yl)amino)-1-(6-((1-methylpiperidin-4-yl)oxy)pyridin-2-yl)-1,2-dihydro-3H-pyrazolo[3,4-d]pyrimidin-3-one (45 mg, 0.079 mmol, 15% yield) as an off-white solid.

### Example 254. Synthesis of 2-allyl-6-(1-methyl-1H-indazol-5-ylamino)-1-(2-{1-[(²H₃)methyl]-4-piperidyloxy}-4-pyrimidinyl)-1,2-dihydro-3H-1,2,5,7-tetraazainden-3-one (Compound 383)

This compound was made using a similar method as described in the synthesis of 2-allyl-6-((1-methyl-1H-indazol-5-yl)amino)-1-(6-((1-(methyl-d3)piperidin-4-yl)oxy)pyridin-2-yl)-1,2-dihydro-3H-pyrazolo[3,4-d]pyrimidin-3-one using 2-allyl-6-((1-methyl-1H-indazol-5-yl)amino)-1-(2-(piperidin-4-yloxy)pyrimidin-4-yl)-1,2-dihydro-3H-pyrazolo[3,4-d]pyrimidin-3-one (470 mg). Yield 98 mg.

### Example 255. Synthesis of 2-allyl-6-((6-(2-fluoroethoxy)pyridin-3-yl)amino)-1-(6-(piperidin-4-yloxy)pyridin-2-yl)-1,2-dihydro-3H-pyrazolo[3,4-d]pyrimidin-3-one (Compound 384) and 6-((1-acetyl-1H-indol-5-yl)amino)-2-allyl-1-(6-((1-methylpiperidin-4-yl)oxy)pyridin-2-yl)-1,2-dihydro-3H-pyrazolo[3,4-d]pyrimidin-3-one (Compound 392)

### 1-(5-nitro-1H-indol-1-yl)ethan-1-one

To a stirred solution of 5-nitro-1H-indole (1.5 g, 9.25 mmol) in Dichloromethane (20 ml) were added triethylamine (6.69 ml, 46.3 mmol) and DMAP (0.565 g, 4.63 mmol) at 25 °C. The mixture was stirred 20 min. Then acetyl chloride (1.980 ml, 27.8 mmol) was dissolved in 5 ml of DCM and added dropwise to reaction mixture at 0 °C. The resulting reaction mixture was stirred at 70 °C for 16 h. The progress of reaction was monitored by TLC. After completion of the reaction, the reaction mixture was concentrated under reduced pressure. The crude product was purified by flash column chromatography (silica-gel, mesh size 60-120) using Ethyl acetate-hexane (20 to 21%) as an eluent to obtain 1-(5-nitro-1H-indol-1-yl)ethan-1-one (1.6 g, 7.13 mmol, 77% yield) as an yellow solid.

### 1-(5-amino-1H-indol-1-yl)ethan-1-one

To a degassed solution of 1-(5-nitro-1H-indol-1-yl)ethan-1-one (1.6 g, 7.84 mmol) in MeOH (30 mL) was added 10% Pd/C (0.834 g, 0.784 mmol) under an inert atmosphere. Then the reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure to give 1-(5-amino-1H-indol-1-yl)ethan-1-one (450 mg, 2.325 mmol, 29.7% yield).

### tert-butyl 4-((6-(6-((1-acetyl-1H-indol-5-yl)amino)-2-allyl-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (0.9 g, 1.696 mmol) in acetic acid (5 ml) was added 1-(5-amino-1H-indol-1-yl)ethan-1-one (0.355 g, 2.035 mmol) at room temperature. The reaction mixture was subjected to stirring at 70 °C for 16 h. The progress of reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure to give crude tert-butyl 4-((6-(6-((1-acetyl-1H-indol-5-yl)amino)-2-allyl-3-oxo-2,3-dihydro-1H-pyrazolo[3,4-d]pyrimidin-1-yl)pyridin-2-yl)oxy)piperidine-1-carboxylate (700 mg, 0.784 mmol, 46.2% yield) as a brown solid.

### 2-allyl-6-((6-(2-fluoroethoxy)pyridin-3-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(6-((1-acetyl-1H-indol-5-yl)amino)-2-allyl-3-oxo-2,3-dihydro-1H-pyrazolo[3,4-d]pyrimidin-1-yl)pyridin-2-yl)oxy)piperidine-1-carboxylate (270 mg, 0.432 mmol) in 1,4-dioxane (5 mL), was added 4N HCl in 1,4-dioxane (15.79 mg, 0.432 mmol) at 0 °C. The resulting reaction mixture was stirred at room temperature for 2 h. The progress of reaction was monitored by TLC. After completion of the reaction, the reaction mixture was concentrated under reduced pressure to give crude compound which was purified by prep. HPLC (X-Select C18 (19*250, 5 mL), Mobile phase A: 0.1 M FA in H₂O, Mobile phase B: acetonitrile) to give 6-((1-acetyl-1H-indol-5-yl)amino)-2-allyl-1-(6-(piperidin-4-yloxy)pyridin-2-yl)-1,2-dihydro-3H-pyrazolo[3,4-d]pyrimidin-3-one (15 mg, 0.028 mmol, 35.3% yield) as an off white solid.

### 6-((1-acetyl-1H-indol-5-yl)amino)-2-allyl-1-(6-((1-methylpiperidin-4-yl)oxy)pyridin-2-yl)-1,2-dihydro-3H-pyrazolo[3,4-d]pyrimidin-3-one

To a stirred solution of 6-((1-acetyl-1H-indol-5-yl)amino)-2-allyl-1-(6-(piperidin-4-yloxy)pyridin-2-yl)-1,2-dihydro-3H-pyrazolo[3,4-d]pyrimidin-3-one (200 mg, 0.381 mmol) in THF (10 mL) was added 37% aq. formaldehyde (1 mL) at 25 °C. The reaction mixture stirred at 25 °C for 5 min. Then was added sodium triacetoxyborohydride (242 mg, 1.144 mmol) portion-wise, after the complete addition of STAB, the reaction mixture was stirred at 25 °C for 20 minutes. The progress of the reaction was monitored by TLC and LCMS. After completion of reaction, the reaction mixture was quenched with 1N NaOH (10 mL) and extracted in ethyl acetate. The combined organic layers was concentrated under vacuum to give crude compound which was purified by prep. HPLC X-Select C18 (19*250, 5 mL), Mobile phase A: 0.1 M FA in H₂O, Mobile phase B: acetonitrile) to give 6-((1-acetyl-1H-indol-5-yl)amino)-2-allyl-1-(6-((1-methylpiperidin-4-yl)oxy)pyridin-2-yl)-1,2-dihydro-3H-pyrazolo[3,4-d]pyrimidin-3-one (28 mg, 0.051 mmol, 10.27% yield) as an off-white solid.

### Example 256. Synthesis of 2-allyl-1-[6-(4-piperidyloxy)-2-pyridyl]-6-(1-propyl-1H-indazol-5-ylamino)-1,2-dihydro-3H-1,2,5,7-tetraazainden-3-one (Compound 390)

This compound was made using a similar method as described in the synthesis of 2-allyl-6-[1-(3-fluoropropyl)-1H-indazol-5-ylamino]-1-[6-(4-piperidyloxy)-2-pyridyl]-1,2-dihydro-3H-1,2,5,7-tetraazainden-3-one using tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate and 1-cyclopropyl-1H-indazol-5-amine. Yield 31 mg.

### Example 257. Synthesis of 2-allyl-1-[6-(1-methyl-4-piperidyloxy)-2-pyridyl]-6-(1-propyl-1H-indazol-5-ylamino)-1,2-dihydro-3H-1,2,5,7-tetraazainden-3-one (Compound 385)

This compound was made using a similar method as described in the synthesis of 2-allyl-6-[1-(3-fluoropropyl)-1H-indazol-5-ylamino]-1-[6-(4-piperidyloxy)-2-pyridyl]-1,2-dihydro-3H-1,2,5,7-tetraazainden-3-one using 2-allyl-1-[6-(4-piperidyloxy)-2-pyridyl]-6-(1-propyl-1H-indazol-5-ylamino)-1,2-dihydro-3H-1,2,5,7-tetraazainden-3-one (400 mg). Yield 92 mg.

### Example 258. Synthesis of 2-allyl-6-((2-(tert-butyl)-2H-indazol-5-yl)amino)-1-(6-(piperidin-4-yloxy)pyridin-2-yl)-1,2-dihydro-3H-pyrazolo[3,4-d]pyrimidin-3-one (Compound 395) and 2-allyl-6-((2-(tert-butyl)-2H-indazol-5-yl)amino)-1-(6-((1-methylpiperidin-4-yl)oxy)pyridin-2-yl)-1,2-dihydro-3H-pyrazolo[3,4-d]pyrimidin-3-one (Compound 396)

### 2-(tert-butyl)-5-nitro-2H-indazole

To a stirred solution of 5-nitro-1H-indazole (2.5 g, 15.32 mmol) in DMF (10 mL) was added potassium carbonate (6.35 g, 46.0 mmol) at 0 °C. Then 2-bromo-2-methylpropane (1.00 g, 153 mmol) was added to the reaction mixture under argon atmosphere. The resulting reaction mixture was stirred at 100 °C for 6 h in a miniclave. The progress of reaction was monitored by TLC. After completion of the reaction, the reaction mixture was diluted with cold water and extracted with ethyl acetate. The combined organic layer was washed with cold water, dried over anhydrous sodium sulphate, concentrated under reduced pressure. The crude material was purified by column chromatography using 30-40% ethyl acetate-hexane as an eluent to give 2-(tert-butyl)-5-nitro-2H-indazole (2 g, 8.85 mmol, 57.7% yield) as an off-white solid. The structure was confirmed by 1D-NOE data.

### 2-(tert-butyl)-2H-indazol-5-amine

To a degassed solution of 2-(tert-butyl)-5-nitro-2H-indazole (2 g, 9.12 mmol) in MeOH (10 ml) was added 10% Pd—C (100 mg, 2.039 mmol) under inert atmosphere. Then the reaction mixture was stirred under hydrogen atmosphere using hydrogen bladder pressure at room temperature for 16 h. The progress of reaction was monitored by TLC and UPLC. After completion of the reaction, the reaction mixture was filtered under nitrogen atmosphere through a pad of celite and washed with MeOH (2×100 mL). The filtrate was concentrated under reduced pressure to give 2-(tert-butyl)-2H-indazol-5-amine (1.5 g, 6.97 mmol, 76% yield) as a brown solid, which was taken into the next step without further purification.

### tert-butyl 4-((6-(2-allyl-6-((2-(tert-butyl)-2H-indazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (1 g, 1.943 mmol) in acetonitrile (5 mL) was added 2-(tert-butyl)-2H-indazol-5-amine (0.478 g, 2.53 mmol) at room temperature. The reaction mixture was subjected to stirring at 70 °C for 16 h. The progress of reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using methanol-DCM (15-20%) as an eluent to obtain tert-butyl 4-((6-(2-allyl-6-((2-(tert-butyl)-2H-indazol-5-yl)amino)-3-oxo-2,3-dihydro-1H-pyrazolo[3,4-d]pyrimidin-1-yl)pyridin-2-yl)oxy)piperidine-1-carboxylate (1 g, 1.391 mmol, 71.6% yield) as a brown solid.

### 2-allyl-6-((2-(tert-butyl)-2H-indazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((2-(tert-butyl)-2H-indazol-5-yl)amino)-3-oxo-2,3-dihydro-1H-pyrazolo[3,4-d]pyrimidin-1-yl)pyridin-2-yl)oxy)piperidine-1-carboxylate (150 mg, 0.117 mmol) in 1,4-dioxane (5 mL), was added 4N HCl in 1,4-dioxane (1 mL) at 0 °C. The resulting reaction mixture was stirred at room temperature for 4 h. The progress of reaction was monitored by TLC. After completion of the reaction, the reaction mixture was concentrated under reduced pressure. The crude residue was purified by prep. HPLC (X-SELECT C18 (19*150 mm) 5u, 0.1% FA in Water-ACN as a eluent) to give the pure fraction, which upon lyophilization yielded 2-allyl-6-((2-(tert-butyl)-2H-indazol-5-yl)amino)-1-(6-(piperidin-4-yloxy)pyridin-2-yl)-1,2-dihydro-3H-pyrazolo[3,4-d]pyrimidin-3-one. (5.5 mg, 10.05 μmol, 8.57% yield) as an off white-solid.

### 2-allyl-6-((2-(tert-butyl)-2H-indazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((2-(tert-butyl)-2H-indazol-5-yl)amino)-1-(6-(piperidin-4-yloxy)pyridin-2-yl)-1,2-dihydro-3H-pyrazolo[3,4-d]pyrimidin-3-one (900 mg, 1.668 mmol) in THF (20 mL) was added 37% aq. formaldehyde (4.5 mL) at 25 °C. The reaction mixture was stirred at 25 °C for 5 min. Then was added sodium triacetoxyborohydride (1060 mg, 5.00 mmol) portion wise, after complete addition of STAB, the reaction mixture was stirred at 25 °C for 20 minutes. The progress of the reaction was monitored by TLC and LCMS. After completion of the reaction, the reaction mixture was quenched with TFA followed by NH₃ in MeOH and diluted with water and extracted with 10% MeOH in DCM (2×100 mL). The combined organic extracts were washed with NaHCO₃, dried over anhydrous Na₂SO₄, filtered and concentrated under reduced pressure to afford crude compound which was purified by prep. HPLC (gemini-NX—C18, 10 MM ABC in water-ACN, Rt-16.5, Flow rate-15 mL) to give 2-allyl-6-((2-(tert-butyl)-2H-indazol-5-yl)amino)-1-(6-((1-methylpiperidin-4-yl)oxy)pyridin-2-yl)-1,2-dihydro-3H-pyrazolo[3,4-d]pyrimidin-3-one (150 mg, 0.268 mmol, 16.08% yield) as an off-white solid.

### Example 259. Synthesis of 2-allyl-6-((1-isopropyl-1H-pyrazolo[3,4-b]pyridin-5-yl)amino)-1-(6-(piperidin-4-yloxy)pyridin-2-yl)-1,2-dihydro-3H-pyrazolo[3,4-d]pyrimidin-3-one (Compound 407) and 2-allyl-6-((1-isopropyl-1H-pyrazolo[3,4-b]pyridin-5-yl)amino)-1-(6-((1-methylpiperidin-4-yl)oxy)pyridin-2-yl)-1,2-dihydro-3H-pyrazolo[3,4-d]pyrimidin-3-one (Compound 406)

### 1-isopropyl-5-nitro-1H-pyrazolo[3,4-b]pyridine

To a stirred solution of 5-nitro-1H-pyrazolo[3,4-b]pyridine (700 mg, 4.27 mmol) in DMF (10 mL) was added cesium carbonate (2223 mg, 6.82 mmol) at 0 °C and, then 2-bromopropane (0.481 ml, 5.12 mmol) was added to the reaction mixture under argon atmosphere. The resulting reaction mixture was stirred at 25 °C for 16 h. The progress of reaction was monitored by TLC. After completion of the reaction, the reaction mixture was diluted with cold water and the precipitated solid was filtered and dried. The crude material was purified by flash column chromatography (silica gel, 100-200 mesh size) using methanol-DCM (15-20%) as an eluent to afford 1-isopropyl-5-nitro-1H-pyrazolo[3,4-b]pyridine (450 mg, 2.160 mmol, 50.7% yield) as a yellow solid. The structure was confirmed by 1D NOE.

### 1-isopropyl-1H-pyrazolo[3,4-b]pyridin-5-amine

To a degassed solution of 1-isopropyl-5-nitro-1H-pyrazolo[3,4-b]pyridine (450 mg, 2.182 mmol) in MeOH (10 ml) was added 10% Pd/C (100 mg, 2.039 mmol) under inert atmosphere. The reaction mixture was stirred under hydrogen atmosphere using hydrogen bladder pressure at room temperature for 16 hours. The progress of reaction was monitored by TLC and UPLC. After completion of the reaction, the reaction mixture was filtered under nitrogen atmosphere through a pad of celite and washed with MeOH (2×100 mL). The filtrate was concentrated under reduced pressure to give 1-isopropyl-1H-pyrazolo[3,4-b]pyridin-5-amine (350 mg, 1.887 mmol, 86% yield) as a black gummy solid, which was taken to the next step without further purification.

### tert-butyl 4-((6-(2-allyl-6-((1-isopropyl-1H-pyrazolo[3,4-b]pyridin-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (584 mg, 1.135 mmol) in acetonitrile (5 mL) was added 1-(3-fluoropropyl)-1H-indazol-5-amine (0.225 g, 1.166 mmol) at room temperature. The reaction mixture was subjected to stirring at 70 °C for 16 h. The progress of reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using methanol-DCM (15-20%) as an eluent to obtain tert-butyl 4-((6-(2-allyl-6-((1-isopropyl-1H-pyrazolo[3,4-b]pyridin-5-yl)amino)-3-oxo-2,3-dihydro-1H-pyrazolo[3,4-d]pyrimidin-1-yl)pyridin-2-yl)oxy)piperidine-1-carboxylate (240 mg, 0.375 mmol, 33.1% yield) as a brown solid.

### 2-allyl-6-((1-isopropyl-1H-pyrazolo[3,4-b]pyridin-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-isopropyl-1H-pyrazolo[3,4-b]pyridin-5-yl)amino)-3-oxo-2,3-dihydro-1H-pyrazolo[3,4-d]pyrimidin-1-yl)pyridin-2-yl)oxy)piperidine-1-carboxylate (240 mg, 0.383 mmol) in DCM (5 mL), was added TFA (0.5 mL) at 0 °C. The resulting reaction mixture was stirred at room temperature for 4 h. The progress of reaction was monitored by TLC. After completion of the reaction, the reaction mixture was concentrated under reduced pressure. The crude residue was purified by prep. HPLC (Shimpak C18 (250*19.5 um) Mobile phase A: 10 MM ABC in H₂O, Mobile phase B: acetonitrile) to give 2-allyl-6-((1-isopropyl-1H-pyrazolo[3,4-b]pyridin-5-yl)amino)-1-(6-(piperidin-4-yloxy)pyridin-2-yl)-1,2-dihydro-3H-pyrazolo[3,4-d]pyrimidin-3-one (20 mg, 0.038 mmol, 10% yield) as an off white-solid.

### 2-allyl-6-((1-isopropyl-1H-pyrazolo[3,4-b]pyridin-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-isopropyl-1H-pyrazolo[3,4-b]pyridin-5-yl)amino)-1-(6-(piperidin-4-yloxy)pyridin-2-yl)-1,2-dihydro-3H-pyrazolo[3,4-d]pyrimidin-3-one (190 mg, 0.361 mmol) in THF (10 mL) was added 37% aq. formaldehyde (6 mL) at 25 °C. The reaction mixture stirred at 25 °C for 5 min. Then was added sodium triacetoxyborohydride (229 mg, 1.082 mmol) portion wise. After complete addition of STAB, the reaction mixture was stirred at 25 °C for 20 min. The progress of the reaction was monitored by TLC and LCMS. After completion of the reaction. The reaction mixture was quenched with TFA followed by NH₃ in MeOH and diluted with water and extracted with 10% MeOH in DCM (2×100 mL). The combined organic extracts were washed with NaHCO₃, dried over anhydrous Na₂SO₄, filtered and concentrated under reduced pressure to afford crude compound, which was purified by prep. HPLC (X-select C18,250 mm×19, Mobile phase A: 0.1% FA in H₂O, Mobile phase B: acetonitrile) to give 2-allyl-6-((1-isopropyl-1H-pyrazolo[3,4-b]pyridin-5-yl)amino)-1-(6-((1-methylpiperidin-4-yl)oxy)pyridin-2-yl)-1,2-dihydro-3H-pyrazolo[3,4-d]pyrimidin-3-one (0.110 mmol, 31.3% yield) as an off-white solid.

### Example 260. Synthesis of 2-allyl-6-((1-isopropyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one (Compound 400)

### 1-isopropyl-5-nitro-1H-benzo[d]imidazole

To a stirred solution of 5-nitro-1H-benzo[d]imidazole (1 g, 6.13 mmol) in DMF (15 ml) was added sodium hydride (0.245 g, 6.13 mmol) at 0 °C under inert atmosphere, then was added 2-iodopropane (0.914 ml, 9.19 mmol) at 0 °C. The reaction mixture was stirred at 25 °C for 16 h. The progress of the reaction was monitored by TLC and LCMS. After completion of the reaction, the reaction mixture was quenched with ice-cold water and there appeared a white solid that was filtered and dried under vacuum to give 1-isopropyl-5-nitro-1H-benzo[d]imidazole (800 mg, 2.77 mmol, 45.2% yield).

### 1-isopropyl-1H-benzo[d]imidazol-5-amine

To a stirred solution of 1-isopropyl-5-nitro-1H-benzo[d]imidazole (1.70 g, 8.28 mmol) in MeOH (10 ml) was added 10% Pd—C (0.882 g, 0.828 mmol). The reaction mixture was stirred under H₂ bladder pressure for 16 h at 25 °C. The progress of the reaction was monitored by TLC and LCMS. After completion of the reaction, the reaction mixture was filtered through celite and washed with methanol. The collected organic layers were concentrated under reduced pressure to give 1-isopropyl-1H-benzo[d]imidazol-5-amine (1.5 g, 3.94 mmol, 47.5% yield). The compound was used as such for the next step.

### tert-butyl 4-((6-(2-allyl-6-((1-isopropyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (600 mg, 1.166 mmol) in acetic acid (10 ml) was added 1-isopropyl-1H-benzo[d]imidazol-5-amine (225 mg, 1.283 mmol) at 25 °C. The reaction mixture was stirred at 70 °C for 16 h. The progress of the reaction was monitored by TLC and LCMS. After completion of the reaction, the reaction mixture was concentrated on the rotary evaporator under reduced pressure. The remaining acid was quenched with aq. NaHCO₃ (100 mL) and the mixture was extracted with ethyl acetate (2×500 mL). The combined organic layers were dried over anhydrous Na₂SO₄, concentrated under reduced pressure to give the crude compound which was purified by column chromatography (SiO₂/230-400 mesh; 50-70% EA in n-hexane) to give tert-butyl 4-((6-(2-allyl-6-((1-isopropyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (300 mg, 0.369 mmol, 31.7% yield).

### 2-allyl-6-((1-isopropyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one

To the stirred solution of tert-butyl 4-((6-(2-allyl-6-((1-isopropyl-1H-benzo[d]imidazol-5-yl)amino)-3-oxo-2,3-dihydro-1H-pyrazolo[3,4-d]pyrimidin-1-yl)pyridin-2-yl)oxy)piperidine-1-carboxylate (300 mg, 0.479 mmol) in dioxane (5 ml) was added 4M HCl (0.144 ml, 0.575 mmol) in dioxane at 25 °C under inert atmosphere. Then the mixture was stirred at 25 °C for 16 h. The progress of the reaction was monitored by TLC and LCMS. After completion of the reaction, reaction mixture was evaporated under reduced pressure to give a crude material which was washed with n-hexane and dried under vacuum to give 2-allyl-6-((1-isopropyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (42 mg, 0.080 mmol, 16.67% yield).

### 2-allyl-6-((1-isopropyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-isopropyl-1H-benzo[d]imidazol-5-yl)amino)-1-(6-(piperidin-4-yloxy)pyridin-2-yl)-1,2-dihydro-3H-pyrazolo[3,4-d]pyrimidin-3-one (240 mg, 0.457 mmol) in THF (5 ml) was added formaldehyde (0.102 ml, 1.370 mmol) at 25 °C and the mixture was stirred for 1 hour. Then STAB (290 mg, 1.370 mmol) was added at 0 °C and the mixture stirred for 2 hours. Progress of the reaction was monitored by TLC and LCMS. After completion of the reaction, the reaction mixture was quenched with ice cold water and extracted with ethyl acetate (3×50 mL). The combined organic layers was washed with brine, dried over anhydrous Na₂SO₄ and concentrated under reduced pressure to give crude product which was purified by prep HPLC (X Select C18, 19×250), Mobile phase A: 0.1% FA in H₂O, Mobile phase B: acetonitrile) to give 2-allyl-6-((1-isopropyl-1H-benzo[d]imidazol-5-yl)amino)-1-(6-((1-methylpiperidin-4-yl)oxy)pyridin-2-yl)-1,2-dihydro-3H-pyrazolo[3,4-d]pyrimidin-3-one (2 mg, 3.62 μmol, 0.79% yield).

### Example 261. Synthesis of 2-allyl-6-((4-(2-fluoroethoxy)phenyl)amino)-1-(2-(piperidin-4-yloxy)pyrimidin-4-yl)-1,2-dihydro-3H-pyrazolo[3,4-d]pyrimidin-3-one (Compound 391) and 2-allyl-6-((4-(2-fluoroethoxy)phenyl)amino)-1-(2-((1-methylpiperidin-4-yl)oxy)pyrimidin-4-yl)-1,2-dihydro-3H-pyrazolo[3,4-d]pyrimidin-3-one (Compound 388)

### 1-(2-fluoroethoxy)-4-nitrobenzene

To a stirred solution of 4-nitrophenol (800 mg, 5.75 mmol) in DMF (10 ml) was added K₂CO₃ (2384 mg, 17.25 mmol) followed by the addition of 1-bromo-2-fluoroethane (876 mg, 6.90 mmol) at 25 °C under inert atmosphere. The mixture was then stirred at 100 °C for 16 h. The progress of the reaction was monitored by TLC and LCMS. After completion of the reaction, reaction mixture quenched with ice cold water and there appeared a solid that was filtered and dried under vacuum to give 1-(2-fluoroethoxy)-4-nitrobenzene (900 mg, 4.86 mmol, 85% yield) as white solid.

### 4-(2-fluoroethoxy)aniline

To a stirred solution of 1-(2-fluoroethoxy)-4-nitrobenzene (900 mg, 4.86 mmol) in ethanol (10 ml) was added Pd—C (517 mg, 4.86 mmol) at rt under inert atmosphere. The mixture was then stirred under H₂ bladder pressure for 16 h. The progress of the reaction was monitored by TLC and LCMS. After completion of the reaction, reaction mixture was filtered using celite bed and the collected fractions were concentrated under reduced pressure to give 4-(2-fluoroethoxy)aniline (700 mg, 4.51 mmol, 93% yield).

### tert-butyl 4-((4-(2-allyl-6-((4-(2-fluoroethoxy)phenyl)amino)-3-oxo-2,3-dihydro-1H-pyrazolo[3,4-d]pyrimidin-1-yl)pyrimidin-2-yl)oxy)piperidine-1-carboxylate

To a stirred solution of tert-butyl 4-((4-(2-allyl-6-(methylsulfonyl)-3-oxo-2,3-dihydro-1H-pyrazolo[3,4-d]pyrimidin-1-yl)pyrimidin-2-yl)oxy)piperidine-1-carboxylate (700 mg, 1.317 mmol) in acetic acid (2 ml) was added 4-(2-fluoroethoxy)aniline (245 mg, 1.580 mmol) at rt under inert atmosphere. The mixture was stirred at rt for 16 h. The progress of the reaction was monitored by TLC and LCMS. After completion of the reaction, the solvent was evaporated under reduced pressure. To the residue was added ice and aq. NaHCO₃ (10 mL). A solid precipitated and was filtered and then triturated with mixture of solvent MTBE and n-hexane to give tert-butyl 4-((4-(2-allyl-6-((4-(2-fluoroethoxy)phenyl)amino)-3-oxo-2,3-dihydro-1H-pyrazolo[3,4-d]pyrimidin-1-yl)pyrimidin-2-yl)oxy)piperidine-1-carboxylate (350 mg, 0.519 mmol, 39.4% yield) as an off-white solid.

### 2-allyl-6-((4-(2-fluoroethoxy)phenyl)amino)-1-(2-(piperidin-4-yloxy)pyrimidin-4-yl)-1,2-dihydro-3H-pyrazolo[3,4-d]pyrimidin-3-one

To a stirred solution of tert-butyl 4-((4-(2-allyl-6-((4-(2-fluoroethoxy)phenyl)amino)-3-oxo-2,3-dihydro-1H-pyrazolo[3,4-d]pyrimidin-1-yl)pyrimidin-2-yl)oxy)piperidine-1-carboxylate (360 mg, 0.593 mmol) in 1,4-dioxane (5 ml) was added 4M HCl in 1,4-dioxane (0.297 ml, 1.187 mmol) at 0 °C under inert atmosphere. The mixture was then stirred at 25 °C for 16 h. The progress of the reaction was monitored by TLC and LCMS. After completion of the reaction, reaction mixture was concentrated under reduced pressure and the residue was washed with n-hexane and further purified by Prep HPLC (X Select C18, 19×250, Mobile phase A: 0.1% FA in H₂O, Mobile phase B: acetonitrile) to give 2-allyl-6-((4-(2-fluoroethoxy)phenyl)amino)-1-(2-(piperidin-4-yloxy)pyrimidin-4-yl)-1,2-dihydro-3H-pyrazolo[3,4-d]pyrimidin-3-one (38.8 mg, 18.59% yield).

### 2-allyl-6-((4-(2-fluoroethoxy)phenyl)amino)-1-(2-((1-methylpiperidin-4-yl)oxy)pyrimidin-4-yl)-1,2-dihydro-3H-pyrazolo[3,4-d]pyrimidin-3-one

To a stirred solution of 2-allyl-6-((4-(2-fluoroethoxy)phenyl)amino)-1-(2-((1-methylpiperidin-4-yl)oxy)pyrimidin-4-yl)-1,2-dihydro-3H-pyrazolo[3,4-d]pyrimidin-3-one (210 mg, 0.403 mmol, 89% yield) in THF (3 ml) was added 37% aq. formaldehyde (68.9 mg, 2.270 mmol) at 25 °C. The reaction mixture was stirred at 25 °C for 5 min, then STAB (289 mg, 1.362 mmol) was added portion wise, under inert atmosphere. After complete addition of STAB, the reaction mixture was stirred at 25 °C for 30 min. The progress of the reaction was monitored by TLC and LCMS. After completion of the reaction, the reaction mixture was quenched with TFA and the excess TFA was quenched with Ammonia in methanol. The reaction mixture was diluted with water and extracted with 10% MeOH-DCM. The combined organic extracts was washed with brine, dried over anhydrous sodium sulphate, filtered, and concentrated under reduced pressure to give the crude compound which was purified by prep HPLC (X Select C18, 19×250, Mobile phase A: 0.1% FA in H₂O, Mobile phase B: acetonitrile) to give 2-allyl-6-((4-(2-fluoroethoxy)phenyl)amino)-1-(2-((1-methylpiperidin-4-yl)oxy)pyrimidin-4-yl)-1,2-dihydro-3H-pyrazolo[3,4-d]pyrimidin-3-one (110 mg, 0.211 mmol, 36.6% yield).

### Example 262. Synthesis of 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one (Compound 359) and 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one (Compound 389)

### tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate

To a solution of tert-butyl 4-((6-(2-allyl-6-(methylsulfonyl)-3-oxo-2,3-dihydro-1H-pyrazolo[3,4-d]pyrimidin-1-yl)pyridin-2-yl)oxy)piperidine-1-carboxylate (800 mg, 1.508 mmol) in AcOH (5 ml) was added 1-methyl-1H-indazol-6-amine (244 mg, 1.658 mmol) at rt under inert atmosphere and stirred at rt for 16 h. The progress of the reaction was monitor by TLC and LCMS. After completion of the reaction, reaction mixture was distilled and aq. NaHCO₃ (50 mL) was added. The mixture was extracted with DCM (2×250 mL). The combined organic layers was washed with brine solution (50 ml) and dried over anhydrous Na₂SO₄, filtered and concentrated under reduced pressure to give the crude compound which was purified by flash column chromatography (silica gel, 100-200 mesh size: ethyl acetate in hexane 60-80%) to give tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate (700 mg, 0.972 mmol, 64.5% yield) as an off-white solid.

### 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-methyl-1H-indazol-6-yl)amino)-3-oxo-2,3-dihydro-1H-pyrazolo[3,4-d]pyrimidin-1-yl)pyridin-2-yl)oxy)piperidine-1-carboxylate (860 mg, 1.439 mmol) in 1,4-dioxane (5 ml) was added 4M HCl in dioxane (0.4 mL, 1.439 mmol) at 0 °C under inert atmosphere. Then the resulting reaction mixture was stirred at room temperature for 16 hours. The progress of the reaction was monitored by TLC and LCMS. After completion of the reaction, the reaction mixture was concentrated under reduced pressure. The crude residue was triturated with n-hexane (40 mL) to give 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one (700 mg, 1.408 mmol, 97% yield) as an off-white solid. 50 mg of the above compound was taken and purified by prep HPLC (Column: X-select CSH C18, Mobile Phase A: Ammonium acetate in water, Mobile Phase B: acetonitrile, Flowrate: 15.0 mL/Min, Rt-10.8) to give the pure compound (21.86 mg).

### 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-methyl-1H-indazol-6-yl)amino)-1-(6-(piperidin-4-yloxy)pyridin-2-yl)-1,2-dihydro-3H-pyrazolo[3,4-d]pyrimidin-3-one (700 mg, 1.407 mmol) in THF (5 ml) was added 37% aq. formaldehyde (0.7 mL, 7.03 mmol) followed by the addition of STAB (895 mg, 4.22 mmol) portion wise. The reaction mixture was stirred at 25 °C for 2 h. The progress of the reaction was monitored by TLC and LCMS. After completion of the reaction, the reaction mixture was quenched with NH₃ in MeOH followed by TFA at rt. The mixture was diluted with water and extracted with 10% MeOH in DCM (2×100 mL). The combined organic layers was dried over anhydrous Na₂SO₄ and concentrated under reduced pressure to give the crude compound which was purified by prep HPLC (Column: X-select CSH C18, Mobile Phase A: Ammonium acetate in water, Mobile Phase B: acetonitrile, Flowrate: 15.0 mL/Min, Rt-10.8) to give 2-allyl-6-((1-methyl-1H-indazol-6-yl)amino)-1-(6-((1-methylpiperidin-4-yl)oxy)pyridin-2-yl)-1,2-dihydro-3H-pyrazolo[3,4-d]pyrimidin-3-one (96 mg, 0.187 mmol, 13.3% yield) as an off-white solid.

### Example 263. Synthesis of 2-allyl-6-((1-isopropyl-1H-benzo[d][1,2,3]triazol-5-yl)amino)-1-(6-(piperidin-4-yloxy)pyridin-2-yl)-1,2-dihydro-3H-pyrazolo[3,4-d]pyrimidin-3-one (Compound 408) and 2-allyl-6-((1-isopropyl-1H-benzo[d][1,2,3]triazol-5-yl)amino)-1-(6-((1-methylpiperidin-4-yl)oxy)pyridin-2-yl)-1,2-dihydro-3H-pyrazolo[3,4-d]pyrimidin-3-one (Compound 409)

### N1-isopropyl-4-nitrobenzene-1,2-diamine

To a stirred solution of 2-fluoro-5-nitroaniline (500 mg, 3.20 mmol) and propan-2-amine (0.273 mL, 3.20 mmol) in DMSO (5 mL) was added triethylamine (0.446 mL, 3.20 mmol) at 0 °C and the mixture was stirred for 16 h at room temperature. The progress of reaction was monitored by TLC. After completion of the reaction, the reaction mixture was quenched with ice-cold water and the precipitate obtained was collected by filtration. The precipitate was then dissolved in DCM and purified by flash column chromatography (silica gel, 60-120 using ethyl acetate-hexane (20 to 70%) as an eluent to obtain N1-isopropyl-4-nitrobenzene-1,2-diamine (550 mg, 2.68 mmol, 84% yield) as a pale brown solid.

### 1-isopropyl-5-nitro-1H-benzo[d][1,2,3]triazole

To a stirred solution of N1-isopropyl-4-nitrobenzene-1,2-diamine (1 g, 5.12 mmol) in DMF (10 mL) were added acetic acid (6 mL) at 0 °C and sodium nitrite (0.530 g, 7.68 mmol). The reaction mixture was stirred for 1 h at room temperature. The progress of reaction was monitored by TLC. After completion of the reaction, the reaction mixture was quenched with ice-cold water and the precipitate obtained was collected by filtration and dried in vacuum to give 1-isopropyl-5-nitro-1H-benzo[d][1,2,3]triazole (1 g, 4.36 mmol, 85% yield) as an orange solid.

### 1-isopropyl-1H-benzo[d][1,2,3]triazol-5-amine

To a degassed solution of 1-isopropyl-5-nitro-1H-benzo[d][1,2,3]triazole (500 mg, 2.425 mmol) in MeOH (10 ml) was added 10% Pd/C (160 mg, 2.74 mmol) under an inert atmosphere. The reaction mixture was stirred under a hydrogen atmosphere using hydrogen bladder pressure at room temperature for 16 h. The progress of the reaction was monitored by TLC and UPLC. After completion of the reaction, the reaction mixture was filtered under nitrogen atmosphere through a pad of celite and washed with MeOH (2×100 mL). The filtrate was concentrated under reduced pressure (bath temperature: 49 °C) to afford 1-isopropyl-1H-benzo[d][1,2,3]triazol-5-amine (410 mg, 2.234 mmol, 92% yield) as an brown solid, which was taken into the next step without further purification.

### tert-butyl 4-((6-(2-allyl-6-((1-isopropyl-1H-benzo[d][1,2,3]triazol-5-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (500 mg, 0.972 mmol) in acetonitrile (10 mL) was added 1-isopropyl-1H-benzo[d][1,2,3]triazol-5-amine (342 mg, 1.943 mmol) at room temperature. The reaction mixture was subjected to stirring at 70 °C for 16 h. The progress of reaction was monitored by LCMS. After the completion of the reaction, the reaction mixture was concentrated under reduced pressure to obtain crude material that was purified by flash column chromatography (silica gel, 100-200) using ethyl acetate-hexane (50 to 100%) as an eluent to obtain tert-butyl 4-((6-(2-allyl-6-((1-isopropyl-1H-benzo[d][1,2,3]triazol-5-yl)amino)-3-oxo-2,3-dihydro-1H-pyrazolo[3,4-d]pyrimidin-1-yl)pyridin-2-yl)oxy)piperidine-1-carboxylate (350 mg, 0.307 mmol, 31.6% yield) as a pale yellow gummy liquid.

### 2-allyl-6-((1-isopropyl-1H-benzo[d][1,2,3]triazol-5-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((1-isopropyl-1H-benzo[d][1,2,3]triazol-5-yl)amino)-3-oxo-2,3-dihydro-1H-pyrazolo[3,4-d]pyrimidin-1-yl)pyridin-2-yl)oxy)piperidine-1-carboxylate (350 mg, 0.335 mmol) in 1,4-dioxane (10 mL), was added 4N HCl in 1,4-dioxane (2.0 mL) at 0 °C. The resulting reaction mixture was stirred at room temperature for 4 h. The progress of reaction was monitored by TLC. After completion of the reaction, the reaction mixture was concentrated under reduced pressure. The crude residue was purified by PREP-HPLC (X-Select C18, Mobile phase A: 0.1% FA in H₂O, Mobile phase B: acetonitrile) to afford 2-allyl-6-((1-isopropyl-1H-benzo[d][1,2,3]triazol-5-yl)amino)-1-(6-(piperidin-4-yloxy)pyridin-2-yl)-1,2-dihydro-3H-pyrazolo[3,4-d]pyrimidin-3-one (10.1 mg, 0.019 mmol, 5.66% yield) as an off white solid.

### 2-allyl-6-((1-isopropyl-1H-benzo[d][1,2,3]triazol-5-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((1-isopropyl-1H-benzo[d][1,2,3]triazol-5-yl)amino)-1-(6-(piperidin-4-yloxy)pyridin-2-yl)-1,2-dihydro-3H-pyrazolo[3,4-d]pyrimidin-3-one (245 mg, 0.279 mmol) in THF (10 mL) was added 37% aq. formaldehyde (20 aq., 2.5 mL) at 25 °C. The reaction mixture was stirred at 25 °C for 5 min. Then sodium triacetoxyborohydride (118 mg, 0.558 mmol) was added portion-wise. After the complete addition, the reaction mixture was stirred at 25 °C for 1 h. The progress of the reaction was monitored by TLC and LCMS. After completion of the reaction, the reaction mixture was quenched with TFA followed by NH₃ in MeOH, after which it was diluted with water and extracted in 10% MeOH in DCM (2×100 mL). The combined organic extract was washed with NaHCO₃, dried over anhydrous Na₂SO₄, filtered, and concentrated under reduced pressure to afford crude compound. The crude was purified by PREP-HPLC (X-Select C18, Mobile phase A: 0.1% FA in H₂O, Mobile phase B: acetonitrile) to afford 2-allyl-6-((1-isopropyl-1H-benzo[d][1,2,3]triazol-5-yl)amino)-1-(6-((1-methylpiperidin-4-yl)oxy)pyridin-2-yl)-1,2-dihydro-3H-pyrazolo[3,4-d]pyrimidin-3-one (62.3 mg, 0.112 mmol, 25% yield) as an off-white solid.

### Example 264. Synthesis of 2-allyl-6-((2-methyl-2H-indazol-6-yl)amino)-1-(6-(piperidin-4-yloxy)pyridin-2-yl)-1,2-dihydro-3H-pyrazolo[3,4-d]pyrimidin-3-one (Compound 393) and 2-allyl-6-((2-methyl-2H-indazol-6-yl)amino)-1-(6-((1-methylpiperidin-4-yl)oxy)pyridin-2-yl)-1,2-dihydro-3H-pyrazolo[3,4-d]pyrimidin-3-one (Compound 398)

### 2-methyl-6-nitro-2H-indazole

To a stirred solution of 6-nitro-2H-indazole (2 g, 12.26 mmol) in Tetrahydrofuran (30 ml) was added potassium carbonate (2.54 g, 18.39 mmol) at 25 °C and the mixture was stirred at the same temperature. Then, iodomethane (0.763 ml, 12.26 mmol) was added to the reaction mixture under an argon atmosphere. The resulting reaction mixture was stirred at 70 °C for 16 h. The progress of the reaction was monitored by TLC. After completion of the reaction, the reaction mixture was concentrated under reduced pressure. The resulting crude material was purified by flash chromatography (SiO₂/230-400 mesh; ~50-80% EtOAc/hexane) to afford 2-methyl-6-nitro-2H-indazole (700 mg, 3.94 mmol, 32.1% yield) as a yellow solid.

### 2-methyl-2H-indazol-6-amine

To a degassed solution of 2-methyl-6-nitro-2H-indazole (700 mg, 3.95 mmol) in EtOH (20 ml) was added 10% Pd/C (546 mg, 3.95 mmol) under an inert atmosphere. Then, the reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure to give 2-methyl-2H-indazol-6-amine (400 mg, 2.60 mmol, 65.8% yield) as a brown gummy solid, which was taken into the next step without further purification.

### tert-butyl 4-((6-(2-allyl-6-((2-methyl-2H-indazol-6-yl)amino)-3-oxo-2,3-dihydro-1H-pyrazolo[3,4-d]pyrimidin-1-yl)pyridin-2-yl)oxy)piperidine-1-carboxylate

To a stirred solution tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (1014 mg, 1.970 mmol) in AcOH (5 ml) was added 2-methyl-2H-indazol-6-amine (290 mg, 1.970 mmol) at room temperature. The reaction mixture was subjected to stirring at 70 °C for 16 h. The progress of the reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure. The crude material was purified by flash column chromatography (silica gel, 100-200 mesh size) using EtOH-hexane (55-60%) as an eluent to obtain tert-butyl 4-((6-(2-allyl-6-((2-methyl-2H-indazol-6-yl)amino)-3-oxo-2,3-dihydro-1H-pyrazolo[3,4-d]pyrimidin-1-yl)pyridin-2-yl)oxy)piperidine-1-carboxylate (280 mg, 0.370 mmol, 18.78% yield) as a brown solid.

### 2-allyl-6-((2-methyl-2H-indazol-6-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((2-methyl-2H-indazol-6-yl)amino)-3-oxo-2,3-dihydro-1H-pyrazolo[3,4-d]pyrimidin-1-yl)pyridin-2-yl)oxy)piperidine-1-carboxylate (350 mg, 0.586 mmol) in DCM (10 ml), was added TFA (66.8 mg, 0.586 mmol) at 0 °C. The resulting reaction mixture was stirred at room temperature for 12 h. The progress of the reaction was monitored by TLC. After completion of the reaction, the reaction mixture was concentrated under reduced pressure. The crude residue was purified by prep. HPLC X-Select C18 (19*250, 5 mL), Mobile phase A: 0.1 M FA in H₂O, Mobile phase B: acetonitrile) to give 2-allyl-6-((2-methyl-2H-indazol-6-yl)amino)-1-(6-(piperidin-4-yloxy)pyridin-2-yl)-1,2-dihydro-3H-pyrazolo[3,4-d]pyrimidin-3-one (250 mg, 0.502 mmol, 86% yield) as an off white-solid.

### 2-allyl-6-((2-methyl-2H-indazol-6-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((2-methyl-2H-indazol-6-yl)amino)-1-(6-(piperidin-4-yloxy)pyridin-2-yl)-1,2-dihydro-3H-pyrazolo[3,4-d]pyrimidin-3-one (280 mg, 0.563 mmol) in THF (10 mL) was added 20% aq. formaldehyde (1 mL) at 25 °C, and then the reaction mixture stirred at 25 °C for 5 min. STAB (358 mg, 1.688 mmol) was added portion-wise and after the complete addition of STAB, the reaction mixture was stirred at 25 °C for 20 min. The progress of the reaction was monitored by TLC and LCMS after completion of the reaction. The reaction mixture was quenched with TFA, followed by NH₃ in MeOH, diluted with water and extracted with 10% MeOH in DCM (2×100 mL). The combined organic extracts were washed with NaHCO₃, dried over anhydrous Na₂SO₄, filtered, and concentrated under reduced pressure to afford crude compound that was purified by prep. HPLC X-Select C18 (19*250, 5 mL), Mobile phase A: 0.1 M FA in H₂O, Mobile phase B: acetonitrile) to give 2-allyl-6-((2-methyl-2H-indazol-6-yl)amino)-1-(6-((1-methylpiperidin-4-yl)oxy)pyridin-2-yl)-1,2-dihydro-3H-pyrazolo[3,4-d]pyrimidin-3-one (112 mg, 0.219 mmol, 38.9% yield) as an off-white solid.

### Example 265. Synthesis of 2-allyl-6-((6-(2-fluoroethoxy)pyridin-3-yl)amino)-1-(6-((1-methylpiperidin-4-yl)oxy)pyridin-2-yl)-1,2-dihydro-3H-pyrazolo[3,4-d]pyrimidin-3-one (Compound 399)

### 2-(2-fluoroethoxy)-5-nitropyridine

To a stirred solution of sodium hydroxide (0.706 g, 17.66 mmol) in Tetrahydrofuran (30 ml) was added 2-fluoroethan-1-ol (0.808 ml, 13.88 mmol) at 25 °C and the mixture was stirred 1 hr at the same temperature. Then 2-chloro-5-nitropyridine (2 g, 12.62 mmol) was added to the reaction mixture under an argon atmosphere. The resulting reaction mixture was stirred at 70 °C for 16 h. The progress of reaction was monitored by TLC. After completion of the reaction, the reaction mixture was concentrated under reduced pressure. Water was added in the crude and stirred for 10 min. A precipitated solid was collected by filtration to give 2-(2-fluoroethoxy)-5-nitropyridine (1.5 g, 7.40 mmol, 58.6% yield) as an off white solid.

### 6-(2-fluoroethoxy)pyridin-3-amine

To a degassed solution of 2-(2-fluoroethoxy)-5-nitropyridine (1.5 g, 8.06 mmol) in EtOH (20 ml) was added 10% Pd/C (1 g, 0.940 mmol) under an inert atmosphere. Then the reaction mixture was stirred under a hydrogen atmosphere using hydrogen bladder pressure at room temperature for 16 h. The progress of the reaction was monitored by TLC and UPLC. After completion of the reaction, the reaction mixture was filtered under a nitrogen atmosphere through a pad of celite and washed with MeOH (2×100 mL). The filtrate was concentrated under reduced pressure to give 6-(2-fluoroethoxy)pyridin-3-amine (840 mg, 5.25 mmol, 65.2% yield).

### tert-butyl 4-((6-(2-allyl-6-((6-(2-fluoroethoxy)pyridin-3-yl)amino)-3-oxo-2,3-dihydro-1H-pyrazolo[3,4-d]pyrimidin-1-yl)pyridin-2-yl)oxy)piperidine-1-carboxylate

To a stirred solution of tert-butyl 4-((6-(2-allyl-6-(methylsulfinyl)-3-oxo-2,3-dihydro-1H-pyrazolo[3,4-d]pyrimidin-1-yl)pyridin-2-yl)oxy)piperidine-1-carboxylate (362 mg, 0.704 mmol) in acetonitrile (5 ml) was added 6-(2-fluoroethoxy)pyridin-3-amine (100 mg, 0.640 mmol) at room temperature. The reaction mixture was subjected to stirring at 70 °C for 16 h. The progress of reaction was monitored by TLC. After completion of the reaction, the reaction mixture was diluted with water and extracted with 10% methanol-DCM (50 mL×2). The combined organic layer was dried over Na₂SO₄, filtered, and concentrated under reduced pressure to give tert-butyl 4-((6-(2-allyl-6-((6-(2-fluoroethoxy)pyridin-3-yl)amino)-3-oxo-2,3-dihydro-1H-pyrazolo[3,4-d]pyrimidin-1-yl)pyridin-2-yl)oxy)piperidine-1-carboxylate (300 mg, 0.223 mmol, 34.8% yield) as a brown solid.

### 2-allyl-6-((6-(2-fluoroethoxy)pyridin-3-yl)amino)-1-(6-(piperidin-4-yloxy)pyridin-2-yl)-1,2-dihydro-3H-pyrazolo[3,4-d]pyrimidin-3-one

To a stirred solution of tert-butyl 4-((6-(2-allyl-6-((6-(2-fluoroethoxy)pyridin-3-yl)amino)-3-oxo-2,3-dihydro-1H-pyrazolo[3,4-d]pyrimidin-1-yl)pyridin-2-yl)oxy)piperidine-1-carboxylate (200 mg, 0.330 mmol) in DCM (10 ml), was added 4M HCl in 1,4-dioxane (2 ml, 0.330 mmol) at 0 °C. The resulting reaction mixture was stirred at room temperature for 2 h. The progress of reaction was monitored by TLC. After completion of the reaction, the reaction mixture was concentrated under reduced pressure to give 2-allyl-6-((6-(2-fluoroethoxy)pyridin-3-yl)amino)-1-(6-(piperidin-4-yloxy)pyridin-2-yl)-1,2-dihydro-3H-pyrazolo[3,4-d]pyrimidin-3-one (136 mg, 0.27 mmol, 75% yield) as an brown solid.

### 2-allyl-6-((6-(2-fluoroethoxy)pyridin-3-yl)amino)-1-(6-((1-methylpiperidin-4-yl)oxy)pyridin-2-yl)-1,2-dihydro-3H-pyrazolo[3,4-d]pyrimidin-3-one

To a stirred solution of 2-allyl-6-((6-(2-fluoroethoxy)pyridin-3-yl)amino)-1-(6-(piperidin-4-yloxy)pyridin-2-yl)-1,2-dihydro-3H-pyrazolo[3,4-d]pyrimidin-3-one (200 mg, 0.395 mmol) in THF (10 mL) was added aq. formaldehyde (1 mL) at 25 °C. The reaction mixture was stirred at 25 °C for 5 min. Then was added sodium tri acetoxy borohydride (84 mg, 0.395 mmol) portion-wise. After the complete addition of STAB, the reaction mixture was stirred at 25 °C for 20 min. The progress of the reaction was monitored by TLC and LCMS. The reaction mixture was quenched with 1N NaOH (10 mL) and extracted with Ethyl acetate. The combined organic layers was concentrated over vacuum to give crude compound which was purified by prep. HPLC X-Select C18 (19*250, 5 mL), Mobile phase A: 0.1 M FA in H₂O, Mobile phase B: acetonitrile) to give 2-allyl-6-((6-(2-fluoroethoxy)pyridin-3-yl)amino)-1-(6-((1-methylpiperidin-4-yl)oxy)pyridin-2-yl)-1,2-dihydro-3H-pyrazolo[3,4-d]pyrimidin-3-one (33.7 mg, 0.063 mmol, 16.51% yield) as an off-white solid.

### Example 266. Synthesis of Intermediates

### 1-(2-chloropyrimidin-4-yl)-6-(methylsulfanyl)-2-(prop-2-en-1-yl)-1H,2H,3H-pyrazolo[3,4-d]pyrimidin-3-one

A stirred suspension of 6-(methylsulfanyl)-2-(prop-2-en-1-yl)-1H,2H,3H-pyrazolo[3,4-d]pyrimidin-3-one (500 mg, 2.25 mmol, 1.0 equiv.), 2,4-dichloropyrimidine (335 mg, 2.25 mmol, 1.0 equiv.), and cesium carbonate (733 mg, 2.25 mmol, 1.0 equiv.) in dry DMF was heated at 75 °C for 21 h. The reaction mixture was then diluted with ethyl acetate (30 ml) and washed successively with NaCl (30 ml, 15% aq.) and sat. brine (30 ml), then filtered through a phase-separator and concentrated under reduced pressure to give a yellow residue, which was purified by flash chromatography (SiO₂, 0-40% Ethyl acetate in petroleum ether) to yield the title compound (413 mg, 55%) as a white solid.

### tert-butyl-4-({4-[6-(methylsulfanyl)-3-oxo-2-(prop-2-en-1-yl)-1H,2H,3H-pyrazolo[3,4-d]pyrimidin-1-yl]pyrimidin-2-yl}amino)piperidine-1-carboxylate

Diisopropylethylamine (208 μl, 1.19 mmol, 2.0 equiv.) was added to a solution of 1-(2-chloropyrimidin-4-yl)-6-(methylsulfanyl)-2-(prop-2-en-1-yl)-1H,2H,3H-pyrazolo[3,4-d]pyrimidin-3-one (200 mg, 0.60 mmol, 1.0 equiv.) and tert-butyl 4-aminopiperidine-1-carboxylate (180 mg, 0.90 mmol, 1.5 equiv.) in a mixture of THF (5 ml) and DMF (1 ml) and the resulting solution was heated at 80-90 °C for 40 h at which point LCMS indicated full conversion of the chloropyrimidine starting material. The mixture was diluted with ethyl acetate (30 ml) and washed successively with NaCl (3×30 ml, 15% aq. adjusted to pH 5 with KH₂PO₄) and sat. brine (30 ml), then filtered through a phase-separator and concentrated under reduced pressure. The residue was purified by flash chromatography (SiO₂, 0-70% Ethyl acetate in petroleum ether) to yield the title compound (186 mg, 62%) as a colorless film.

### tert-butyl-(3S)-3-(methylamino)piperidine-1-carboxylate

Trifluoroacetic acid anhydride (412 μl, 2.97 mmol, 1.1 equiv.) was added dropwise to stirred a solution of tert-butyl (3S)-3-aminopiperidine-1-carboxylate (540 mg, 2.70 mmol, 1.0 equiv.) and diisopropylethylamine (939 μl, 5.39 mmol, 2.0 equiv.) in DCM (15 ml) at RT. After 30 min, the solution was washed with NaH₂PO₄ (20 ml, 1 M aq.) followed by sat. brine (20 ml), then filtered through a phase-separator and concentrated under reduced pressure to give tert-butyl (3S)-3-(2,2,2-trifluoroacetamido)piperidine-1-carboxylate (769 mg, 96%) as a colorless oil. This intermediate (769 mg, 2.60 mmol, 1.0 equiv.) was dissolved in DMF (8 ml) to which cesium carbonate (1.46 g, 4.47 mmol, 1.7 equiv.) followed by iodomethane (242 μl, 3.89 mmol, 1.5 equiv.) were added. The resulting mixture was stirred at RT for 17 h, when LCMS indicated full conversion to the methylated amide. A solution of lithium hydroxide (3 ml, 2M aq., 6 mmol, 2.3 equiv.) and methanol (2 ml) were then added, and the stirring was continued for 2 h when full conversion of the trifluoroacetamide intermediate was observed by LCMS. The reaction mixture was diluted with ethyl acetate (60 ml) and washed successively with water (2×60 ml) and sat. brine (60 ml), adjusting the aqueous pH to >11 with NaOH. The organic phase was filtered through a phase-separator and concentrated under reduced pressure to give the title compound (441 mg, 76% over 3 steps) as a yellow oil.

### tert-butyl-(3R)-3-(methylamino)piperidine-1-carboxylate

The title compound was prepared in analogy with tert-butyl (3S)-3-(methylamino)piperidine-1-carboxylate using tert-butyl (3R)-3-aminopiperidine-1-carboxylate (500 mg). Yield: 456 mg (85% over 3 steps) as a yellow oil.

### tert-butyl-(3S)-3-[(6-bromopyridin-2-yl)amino]pyrrolidine-1-carboxylate

This intermediate was prepared with a method similar to that as described in the procedure for making tert-butyl 4-((6-bromopyridin-2-yl)amino)piperidine-1-carboxylate using 2,6-dibromopyridine (254 mg) and tert-butyl (3S)-3-aminopyrrolidine-1-carboxylate (2 equiv.). Yield: 123 mg (33%).

### tert-butyl-(3S)-3-[(6-bromopyridin-2-yl)(methyl)amino]piperidine-1-carboxylate

This intermediate was prepared with a method similar to that as described in the procedure for making tert-butyl 4-((6-bromopyridin-2-yl)amino)piperidine-1-carboxylate using 2,6-dibromopyridine (487 mg) and tert-butyl (3S)-3-aminopyrrolidine-1-carboxylate (1 equiv.). Yield: 138 mg (18%) as a yellow oil.

### tert-buty-(3R)-3-[(6-bromopyridin-2-yl)(methyl)amino]piperidine-1-carboxylate

This intermediate was prepared with a method similar to that as described in the procedure for making tert-butyl 4-((6-bromopyridin-2-yl)amino)piperidine-1-carboxylate using 2,6-dibromopyridine (456 mg) and tert-butyl (3R)-3-aminopyrrolidine-1-carboxylate (1 equiv.). Yield: 252 mg (32%) as a yellow oil.

### tert-butyl-4-[(6-bromopyridin-2-yl)amino]piperidine-1-carboxylate

This intermediate was prepared with a method similar to that as described in the procedure for making tert-butyl 4-((6-bromopyridin-2-yl)amino)piperidine-1-carboxylate using 2,6-dibromopyridine (2 g) and tert-butyl 4-aminopiperidine-1-carboxylate. Yield: 1.45 g (48%).

### tert-butyl-4-[(6-bromopyridin-2-yl)oxy]piperidine-1-carboxylate

This intermediate was prepared with a method similar to that as described in the procedure for making tert-butyl 4-((6-bromopyridin-2-yl)oxy)piperidine-1-carboxylate using 2,6-dibromopyridine (650 mg) and tert-butyl 4-hydroxypiperidine-1-carboxylate (1 equiv.). Yield: 698 mg (71%) as a colorless oil.

### tert-butyl-(3S)-3-[(6-bromopyridin-2-yl)oxy]piperidine-1-carboxylate

This intermediate was prepared with a method similar to that as described in the procedure for making tert-butyl 4-((6-bromopyridin-2-yl)oxy)piperidine-1-carboxylate using 2,6-dibromopyridine (235 mg) and tert-butyl (3S)-3-hydroxypiperidine-1-carboxylate (1 equiv.). Yield: 275 mg (77%) as a colorless oil.

### 2-bromo-6-[(1-methylpiperidin-4-yl)oxy]pyridine

This intermediate was prepared with a method similar to that as described in the procedure for making tert-butyl 4-((6-bromopyridin-2-yl)oxy)piperidine-1-carboxylate using 2,6-dibromopyridine (2 g) and 1-methylpiperidin-4-ol (1 equiv.). Yield: 1.7 g (74%) as a colorless oil.

### tert-butyl-4-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl 4-[(6-bromopyridin-2-yl)oxy]piperidine-1-carboxylate (650 mg). Yield: 717 mg (79%) as a brown oil.

### tert-butyl-(3S)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl (3S)-3-[(6-bromopyridin-2-yl)oxy]piperidine-1-carboxylate (275 mg). Yield: 135 mg (35%) as pale-yellow oil.

### tert-butyl-(3S)-3-({6-[6-(methylsulfanyl)-3-oxo-2-(prop-2-en-1-yl)-1H,2H,3H-pyrazolo[3,4-d]pyrimidin-1-yl]pyridin-2-yl}oxy) pyrrolidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl (3S)-3-[(6-bromopyridin-2-yl)oxy]pyrrolidine-1-carboxylate (484 mg). Yield: 507 mg (74%).

### tert-butyl-(3S)-3-({6-[6-(methylsulfanyl)-3-oxo-2-(prop-2-en-1-yl)-1H,2H,3H-pyrazolo[3,4-d]pyrimidin-1-yl]pyridin-2-yl}amino)pyrrolidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl (3S)-3-[(6-bromopyridin-2-yl)amino]pyrrolidine-1-carboxylate (80 mg). Yield: 90 mg (52%) as pale-yellow oil.

### 2-methyl-1-{6-[(1-methylpiperidin-4-yl)oxy]pyridin-2-yl}-6-(methylsulfanyl)-1H,2H,3H-pyrazolo[3,4-d]pyrimidin-3-one

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using 2-bromo-6-[(1-methylpiperidin-4-yl)oxy]pyridine (500 mg) and 2-methyl-6-(methylsulfanyl)-1H,2H,3H-pyrazolo[3,4-d]pyrimidin-3-one (1 equiv.). Yield: 252 mg (35%) as pale-yellow oil.

### tert-butyl-(3S)-3-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl (3S)-3-[(6-bromopyridin-2-yl)(methyl)amino]piperidine-1-carboxylate (138 mg) and 2-methyl-6-(methylsulfanyl)-1H,2H,3H-pyrazolo[3,4-d]pyrimidin-3-one (1 equiv.). Yield: 193 mg (crude) as pale-yellow oil. Used in the next step without further purification.

### tert-butyl-(3R)-3-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl (3R)-3-[(6-bromopyridin-2-yl)(methyl)amino]piperidine-1-carboxylate (252 mg) and 2-methyl-6-(methylsulfanyl)-1H,2H,3H-pyrazolo[3,4-d]pyrimidin-3-one (1 equiv.). Yield: 345 mg (crude) as pale-yellow oil. Used in the next step without further purification.

### 2-ethyl-1-{6-[(1-methylpiperidin-4-yl)oxy]pyridin-2-yl}-6-(methylsulfanyl)-1H,2H,3H-pyrazolo[3,4-d]pyrimidin-3-one

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using 2-bromo-6-[(1-methylpiperidin-4-yl)oxy]pyridine (800 mg) and 2-ethyl-6-(methylsulfanyl)-1H,2H,3H-pyrazolo[3,4-d]pyrimidin-3-one (1 equiv.). Yield: 886 mg (75%) as pale-yellow oil.

### tert-butyl-4-({6-[2-methyl-6-(methylsulfanyl)-3-oxo-1H,2H,3H-pyrazolo[3,4-d]pyrimidin-1-yl]pyridin-2-yl}oxy)piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl 4-[(6-bromopyridin-2-yl)oxy]piperidine-1-carboxylate (165 mg) and 2-methyl-6-(methylsulfanyl)-1H,2H,3H-pyrazolo[3,4-d]pyrimidin-3-one (1 equiv.). The purified material was triturated with hot heptane to remove remaining bromopyridine not separated by chromatography. Yield: 210 mg (53%) as a pale-yellow powder.

### tert-butyl-4-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using tert-butyl 4-[(6-bromopyridin-2-yl)amino]piperidine-1-carboxylate (1 g). Yield: 914 mg (65%) as yellow solid.

### tert-butyl-4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate

3-chloro-peroxybenzoic acid (102 mg, 77%, 0.45 mmol, 1.1 equiv.) was added to a solution of tert-butyl-4-({6-[6-(methylsulfanyl)-3-oxo-2-(prop-2-en-1-yl)-1H,2H,3H-pyrazolo[3,4-d]pyrimidin-1-yl]pyridin-2-yl}oxy)piperidine-1-carboxylate in dichloromethane (5 ml) and the solution was stirred at room temperature for 1.5 h. Ammonia (200 μl, 28% aq., 5.53 mmol, 8.8 equiv.) was then added and stirring was continued for 18 h. The resulting white suspension was filtered to remove ammonium chlorobenzoate and the filtrate was concentrated under reduced pressure to give 6-amino-1-[6-(piperidin-4-yloxy)pyridin-2-yl]-2-(prop-2-en-1-yl)-1H,2H,3H-pyrazolo[3,4-d]pyrimidin-3-one (172 mg, 91%) as a brown foam.

### tert-butyl-4-[(6-{6-[(1,3-benzothiazol-6-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate; trifluoroacetic acid

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (100 mg) and 6-bromo-1,3-benzothiazole (2 equiv.). Yield: 27 mg TFA salt (18%) as a pale-yellow powder.

### tert-butyl-4-({6-[3-oxo-2-(prop-2-en-1-yl)-6-[(pyridin-3-yl)amino]-1H,2H,3H-pyrazolo[3,4-d]pyrimidin-1-yl]pyridin-2-yl}oxy)piperidine-1-carboxylate

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (204 mg) and 3-bromopyridine (2 equiv.). Yield: 125 mg (54%) as a pale-yellow powder.

### tert-butyl-4-[(6-{6-[(2-methyl-1,3-benzothiazol-6-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (180 mg) and 6-bromo-2-methyl-1,3-benzothiazole (1 equiv.). Yield: 127 mg (54%).

### tert-butyl-4-[(6-{6-[(2-methoxypyridin-4-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (200 mg) and 4-bromo-2-methoxypyridine (1 equiv.). Yield: 288 mg (crude).

### tert-butyl-4-[(6-{6-[(5-methoxypyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (200 mg) and 3-bromo-5-methoxypyridine (1 equiv.). Yield: 300 mg (crude).

### tert-butyl-4-[(6-{6-[(5-fluoropyridin-3-yl)amino]-3-oxo-2-(prop-2-en-1-yl)-1H,2H,3H-pyrazolo[3,4-d]pyrimidin-1-yl}pyridin-2-yl)oxy]piperidine-1-carboxylate

This intermediate was prepared with a method similar to that as described in the procedure for making tert-butyl 4-{6-[6-(2-methoxypyrid-4-ylamino)-3-oxo-2-(prop-2-enyl)-1,2-dihydro-3H-1,2,5,7-tetraazainden-1-yl]pyrid-2-yloxy}piperidine-1-carboxylate using tert-butyl 4-({6-[6-amino-3-oxo-2-(prop-2-en-1-yl)-1H,2H,3H-pyrazolo[3,4-d]pyrimidin-1-yl]pyridin-2-yl}oxy)piperidine-1-carboxylate (204 mg) and 3-bromo-5-fluoropyridine (2 equiv.). Yield: 231 mg, (67%).

### tert-butyl-4-[(6-{6-amino-2-ethyl-3-oxo-1H,2H,3H-pyrazolo[3,4-d]pyrimidin-1-yl}pyridin-2-yl)oxy]piperidine-1-carboxylate

mCPBA (~75%, 142 mg, 617 μmol) was added to a stirred solution of tert-butyl 4-({6-[2-ethyl-6-(methylsulfanyl)-3-oxo-1H,2H,3H-pyrazolo[3,4-d]pyrimidin-1-yl]pyridin-2-yl}oxy)piperidine-1-carboxylate (300 mg, 617 μmol) in dichloromethane (5 mL) at room temperature. The mixture was stirred for 2 hours, then aq. ammonia (0.3 mL, 4.93 mmol) was added to the reaction mixture. The resulting suspension was heated to 40 °C overnight. The precipitate was removed by filtration and the solution was concentrated to solids (315 mg, quant.) that were used without further purification.

### tert-butyl-(3R)-3-({6-[6-(methylsulfanyl)-3-oxo-2-(prop-2-en-1-yl)-1H,2H,3H-pyrazolo[3,4-d]pyrimidin-1-yl]pyridin-2-yl}amino)piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using 6-(methylsulfanyl)-2-(prop-2-en-1-yl)-1H,2H,3H-pyrazolo[3,4-d]pyrimidin-3-one and tert-butyl (3R)-3-[(6-bromopyridin-2-yl)amino]piperidine-1-carboxylate. Yield: 527 mg, 64%.

### tert-butyl-4-[(6-bromopyridin-2-yl)(methyl)amino]piperidine-1-carboxylate

Sodium hydride 60% (192 mg, 4.79 mmol) was added to tert-butyl 4-[(6-bromopyridin-2-yl)amino]piperidine-1-carboxylate (569 mg, 1.6 mmol) under nitrogen in dimethylformamide (15 mL) at rt. The suspension was stirred for one hour, then methyl iodide (497 μL, 7.99 mmol) was added at rt. After 150 minutes the mixture was added to brine (50 mL) and extracted with ethyl acetate (3×20 mL). The combined organic layers were separated, dried using a phase separator and concentrated to residues. The residues were purified by flash chromatography on silica using 0-20% ethyl acetate in petroleum ether. The fractions containing product were combined and concentrated to give colourless oil (420 mg, 71%).

### tert-butyl-4-[methyl({6-[6-(methylsulfanyl)-3-oxo-2-(prop-2-en-1-yl)-1H,2H,3H-pyrazolo[3,4-d]pyrimidin-1-yl]pyridin-2-yl})amino]piperidine-1-carboxylate

This intermediate was prepared with a method similar to that as described in the procedure for making tert-butyl 4-((6-(2-allyl-6-(methylthio)-3-oxo-2,3-dihydro-1H-pyrazolo[3,4-d]pyrimidin-1-yl)pyridin-2-yl)oxy)piperidine-1-carboxylate using 6-(methylsulfanyl)-2-(prop-2-en-1-yl)-1H,2H,3H-pyrazolo[3,4-d]pyrimidin-3-one and tert-butyl 4-[(6-bromopyridin-2-yl)(methyl)amino]piperidine-1-carboxylate. Yield: 55 mg, 78%.

### tert-butyl-(3S)-3-hydroxypyrrolidine-1-carboxylate

Boc-anhydride (1.5 g, 6.9 mmol) was added to a suspension of triethylamine (2.4 mL, 17 mmol) and (3S)-pyrrolidin-3-ol (0.5 g, 5.7 mmol) in dichloromethane (5 mL) at room temperature. After 1 day the material was concentrated, dissolved in ethyl acetate (25 mL) and washed with aq. NaOH (25 mL, 1M), water (25 mL), and brine (25 mL). The organic layer was dried using a phase separator and concentrated to give colourless oil (851 mg, 79%).

### tert-butyl-(3R)-3-hydroxypyrrolidine-1-carboxylate

Boc-anhydride (1.5 g, 6.9 mmol) was added to a suspension of triethylamine (2.4 mL, 17 mmol) and (3R)-pyrrolidin-3-ol (0.5 g, 5.7 mmol) in dichloromethane (5 mL) at room temperature. After 1 day the material was concentrated, dissolved in ethyl acetate (25 mL) and washed with aq. NaOH (25 mL, 1M), water (25 mL), and brine (25 mL). The organic layer was dried using a phase separator and concentrated to give colourless oil (758 mg, 71%).

### Example 267. Wee1A Kinase Binding Assay

We used a LanthaScreen Europium (Eu) Kinase Binding Assay to determine inhibitor affinities (IC₅₀) to Wee1A kinase. The assay utilizes an Alexa Fluor 647-labeled ATP competitive kinase tracer that binds to the ATP binding site of a GST-tagged Wee1A kinase, while a europium (Eu) labeled antibody binds to the GST tag. The proximity of fluorescently labeled kinase tracer and europium (Eu) donor fluorophore antibody leads to fluorescence resonance energy transfer (FRET) to the fluorescence label (acceptor) upon excitation of the Eu (donor). Displacement of tracer from the ATP-binding site by a test compound disturbs the proximity between both labels thus lowering the FRET.

This time resolved-FRET binding assay was performed in white 384-well low volume plates (Greiner, cat #784075), at room temperature in kinase buffer A (KBA; Invitrogen cat #PV3189), consisting of 50 mM HEPES-NaOH (pH 7.5), 0.01% Brij-35, 10 mM MgCl₂, and 1 mM EGTA. 5 μL of compound (diluted in reaction buffer, to 1% DMSO) were added to various wells in the plate, followed by 5 μL each of recombinant human Wee1A kinase (full length Wee1 kinase was expressed by baculovirus in insect cells using a N-terminal GST tag (MW: 99.1 kDa) (Invitrogen cat #PV3817) and LanthaScreen Eu-anti-GST antibody (Invitrogen, cat #PV5594). After this, 5 μL of kinase tracer 178 (Invitrogen, cat #PV5593) was added to the plate and the plate was incubated for 60 minutes at room temperature. The final assay conditions in each well were: 30 nM tracer 178, 5 nM Wee1A kinase and 2 nM Eu-labeled antibody in total assay volume of 15 μL. An Envision 2104 (Perkin-Elmer) Plate Reader with the following time-resolve fluorescence setting was used for performing LanthaScreen kinase binding assay.
Excitation 320 nm (30 nm bandpass)
Kinase Tracer Emission 665 nm (10 nm bandpass)
LanthaScreen Eu-anti-Tag Antibody Emission 615 nm (10 nm bandpass)
Dichroic Mirror Instrument dependent
Delay Time 100 μs
Integration Time 200 μs

To calculate the emission ratio, the acceptor/tracer emission (665 nM) was divided by the antibody/donor emission (615 nM) and the average of duplicate measurement was used for calculations. Data plotting, analysis of binding, and curve fitting was done using Excel add-in XLfit version 5.5.0.5 (IDBS, Guildford, United Kingdom). Results are presented in Table 2, below, where compounds having an IC₅₀ less than or equal to 10 nM are represented as “A”; compounds having an IC₅₀ greater than 10 nM but less than or equal to 100 nM are represented as “B”; compounds having an IC₅₀ greater than 100 nM but less than or equal to 500 nM are represented as “C”; and compounds having an IC₅₀ greater than 500 nM are represented as “D”. “NT” means not tested.

TABLE 2
Wee1A kinase Binding IC₅₀ Values for Exemplary Compounds

| Compound | IC₅₀ |
|---|---|
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | B |
| 163 | B |
| 164 | B |
| 165 | A |
| 166 | A |
| 167 | B |
| 168 | A |
| 169 | A |
| 170 | A |
| 171 | A |
| 172 | A |
| 173 | B |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 204 | A |
| 205 | A |
| 206 | A |
| 207 | A |
| 208 | A |
| 209 | A |
| 211 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 284 | A |
| 285 | A |
| 286 | A |
| 287 | A |
| 288 | A |
| 289 | A |
| 290 | A |
| 291 | A |
| 292 | A |
| 293 | A |
| 294 | B |
| 295 | B |
| 296 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | A |
| 348 | A |
| 349 | A |
| 350 | A |
| 351 | A |
| 352 | A |
| 353 | A |
| 354 | NT |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | A |
| 402 | A |
| 403 | A |
| 404 | A |
| 405 | A |
| 406 | A |
| 407 | A |
| 408 | A |
| 409 | A |

### Example 268. Myt1 Kinase Binding Assay

We used a LanthaScreen Europium (Eu) Kinase Binding Assay to determine the binding affinities (IC₅₀) of compounds to Myt1 kinase. The assay was identical to the Wee1A kinase binding assay disclosed in Example 4, except for the use of recombinant PMYT-1 (full length PMYT-1 was expressed by baculovirus in insect cells using a N-terminal GST tag (MW: 80.8 kDa) (Invitrogen cat #A30984) and LanthaScreen Eu-anti-GST antibody (Invitrogen, cat #PV5594) in place of recombinant human Wee1A kinase and the final concentration of reagents in the assay. The final assay conditions were: 3 nM tracer 178, 2.5 nM PMYT-1 and 1 nM Eu-labeled antibody in total assay volume of 15 μL. Results are presented in Table 3, below, where compounds having an IC₅₀ less than or equal to 20 nM are represented as “A”; compounds having an IC₅₀ greater than 20 nM but less than or equal to 100 nM are represented as “B”; compounds having an IC₅₀ greater than 100 nM but less than or equal to 500 nM are represented as “C”; and compounds having an IC₅₀ greater than 500 nM are represented as “D”. “NT” designates that the compound was not tested in this assay.

TABLE 3
Binding IC₅₀ Values for Exemplary Compounds

| Compound | IC₅₀ |
|---|---|
| 100 | A |
| 101 | C |
| 102 | B |
| 103 | C |
| 104 | A |
| 105 | B |
| 106 | B |
| 107 | B |
| 108 | B |
| 109 | A |
| 110 | B |
| 111 | B |
| 112 | B |
| 113 | B |
| 114 | B |
| 115 | B |
| 116 | D |
| 117 | B |
| 118 | D |
| 119 | C |
| 120 | C |
| 121 | B |
| 122 | C |
| 123 | C |
| 124 | B |
| 125 | C |
| 126 | D |
| 127 | C |
| 128 | C |
| 129 | C |
| 130 | B |
| 131 | C |
| 132 | C |
| 133 | C |
| 134 | C |
| 135 | C |
| 136 | C |
| 137 | B |
| 138 | C |
| 139 | C |
| 140 | C |
| 141 | A |
| 142 | A |
| 143 | B |
| 144 | D |
| 145 | B |
| 146 | C |
| 147 | D |
| 148 | B |
| 149 | D |
| 150 | B |
| 151 | C |
| 152 | B |
| 153 | B |
| 154 | C |
| 155 | C |
| 156 | C |
| 157 | D |
| 158 | B |
| 159 | B |
| 160 | C |
| 161 | C |
| 162 | C |
| 163 | D |
| 164 | D |
| 165 | B |
| 166 | C |
| 167 | C |
| 168 | D |
| 169 | D |
| 170 | D |
| 171 | D |
| 172 | D |
| 173 | D |
| 174 | D |
| 175 | D |
| 176 | D |
| 177 | D |
| 178 | C |
| 179 | D |
| 180 | C |
| 181 | C |
| 182 | D |
| 183 | D |
| 184 | D |
| 185 | D |
| 186 | B |
| 187 | C |
| 188 | C |
| 189 | D |
| 190 | A |
| 191 | A |
| 192 | C |
| 193 | D |
| 194 | A |
| 195 | C |
| 196 | C |
| 197 | B |
| 198 | C |
| 199 | C |
| 200 | C |
| 201 | C |
| 202 | C |
| 204 | B |
| 205 | B |
| 206 | B |
| 207 | B |
| 208 | A |
| 209 | A |
| 211 | B |
| 220 | C |
| 223 | D |
| 226 | D |
| 227 | A |
| 228 | A |
| 229 | B |
| 230 | A |
| 231 | A |
| 232 | A |
| 233 | B |
| 234 | B |
| 235 | B |
| 236 | B |
| 237 | B |
| 238 | B |
| 239 | A |
| 240 | B |
| 241 | A |
| 242 | A |
| 243 | A |
| 244 | B |
| 245 | B |
| 246 | A |
| 247 | A |
| 248 | A |
| 249 | B |
| 250 | B |
| 251 | B |
| 252 | B |
| 253 | B |
| 254 | B |
| 255 | A |
| 256 | A |
| 257 | A |
| 258 | A |
| 259 | A |
| 260 | A |
| 261 | A |
| 262 | B |
| 263 | A |
| 264 | A |
| 265 | A |
| 266 | B |
| 267 | B |
| 268 | A |
| 269 | A |
| 270 | A |
| 271 | A |
| 272 | A |
| 273 | A |
| 274 | A |
| 275 | B |
| 276 | B |
| 277 | B |
| 278 | B |
| 279 | B |
| 280 | A |
| 281 | B |
| 282 | A |
| 284 | A |
| 285 | C |
| 286 | NT |
| 287 | NT |
| 288 | D |
| 289 | C |
| 290 | C |
| 291 | B |
| 292 | B |
| 293 | B |
| 294 | NT |
| 295 | NT |
| 296 | B |
| 297 | C |
| 298 | C |
| 299 | B |
| 300 | B |
| 301 | B |
| 302 | B |
| 303 | A |
| 304 | B |
| 305 | B |
| 306 | B |
| 307 | B |
| 308 | A |
| 309 | A |
| 310 | B |
| 311 | B |
| 312 | C |
| 313 | B |
| 314 | B |
| 315 | B |
| 316 | B |
| 317 | B |
| 318 | B |
| 319 | B |
| 320 | A |
| 321 | B |
| 322 | B |
| 323 | B |
| 324 | B |
| 325 | B |
| 326 | B |
| 327 | B |
| 328 | B |
| 329 | B |
| 330 | C |
| 331 | NT |
| 332 | C |
| 333 | B |
| 334 | B |
| 335 | A |
| 336 | B |
| 337 | C |
| 338 | B |
| 339 | B |
| 340 | C |
| 341 | B |
| 342 | B |
| 343 | C |
| 344 | D |
| 345 | B |
| 346 | B |
| 347 | B |
| 348 | B |
| 349 | NT |
| 350 | C |
| 351 | B |
| 352 | B |
| 353 | B |
| 354 | A |
| 355 | A |
| 356 | A |
| 357 | B |
| 358 | C |
| 359 | NT |
| 360 | B |
| 361 | NT |
| 362 | C |
| 363 | B |
| 364 | B |
| 365 | B |
| 366 | C |
| 367 | C |
| 368 | B |
| 369 | A |
| 370 | A |
| 371 | C |
| 372 | B |
| 373 | A |
| 374 | B |
| 375 | B |
| 376 | A |
| 377 | A |
| 378 | A |
| 379 | C |
| 380 | C |
| 381 | C |
| 382 | C |
| 383 | B |
| 384 | B |
| 385 | A |
| 386 | A |
| 387 | B |
| 388 | B |
| 389 | B |
| 390 | A |
| 391 | B |
| 392 | B |
| 393 | B |
| 394 | A |
| 395 | A |
| 396 | A |
| 397 | B |
| 398 | B |
| 399 | A |
| 400 | A |
| 401 | A |
| 402 | C |
| 403 | B |
| 404 | B |
| 405 | B |
| 406 | A |
| 407 | A |
| 408 | A |
| 409 | A |

### Example 269. Wee1A Kinase and Myt1 Kinase Cellular Assays

### Cell Culture

Each of an ACHN renal cell carcinoma cell line (ATCC) and a DAOY medulloblastoma cell line, and an A427 lung cancer line were cultured in Minimum Essential Medium Eagle supplemented with 10% fetal calf serum (Sigma), 1% Penicillin-Streptomycin and 10 mM HEPES buffer (HyClone). Cell cultures were kept in a humidified incubator at 37 °C and 5% CO₂. Cells were routinely tested for Mycoplasma contamination.

### AlphaLISA Assay

For target engagement assessment of Myt1 kinase inhibition, quantification of Cdk1 phosphorylated on threonine 14 was detected using the AlphaLISA® SureFire® Ultra™ Human Phospho-CDK1 (Thr14) assay (Perkin Elmer). DAOY or ACHN cells were seeded into tissue culture treated 96-well plates (VWR) to a density of 10,000 or 20,000 cells per well respectively. 24 h post seeding cells were treated for 4 h with compounds at concentrations ranging from 7 to 5000 nM. Cells were washed with PBS and lysed in 50 μl AlphaLISA lysis buffer before freezing at −80 C. Ten ul of the lysate was transferred to 384 well plates and incubated with AlphaLISA donor and acceptor beads according to the manufacturer's instructions. Dose response curves and EC₅₀ values were calculated and visualized using GraphPad Prism version 9. Target engagement for Myt1 kinase can also be measured using the same AlphaLISA assay in OVCAR3 cells, an ovarian cancer cell line. NIH:OVCAR-3 (OVCAR3) cells are cultured in RPMI-1640 medium supplemented with 0.01 mg/ml insulin, 20% FBS, 1% Penicillin-Streptomycin and 10 mM HEPES buffer. 14,000 cells per well are seeded into 96 well tissue culture plates in full medium. Twenty-four hours post seeding, cells are treated with compound for 4 hours at concentrations ranging from 0.017 nM to 1000 nM.

Results are presented in Table 4 below where compounds having an EC₅₀ less than or equal to 200 nM are represented as “A”; compounds having an EC₅₀ greater than 200 nM but less than or equal to 500 nM are represented as “B”; compounds having an EC₅₀ greater than 500 nM but less than or equal to 1,000 nM are represented as “C”; and compounds having an EC₅₀ greater than 1,000 nM are represented as “D”.

TABLE 4
CDK1 Thr14 Phosphorylation EC₅₀
Values for Exemplary Compounds

| Compound | EC₅₀ | Compound | EC₅₀ | Compound | EC₅₀ |
|---|---|---|---|---|---|
| 100 | D | 241 | B | 320 | B |
| 104 | B | 242 | C | 321 | C |
| 105 | C | 243 | C | 322 | B* |
| 106 | B | 244 | C | 326 | B* |
| 109 | A | 248 | A | 333 | B* |
| 112 | B | 250 | B | 335 | B |
| 114 | C* | 253 | D | 346 | B* |
| 121 | B | 254 | C | 354 | A* |
| 129 | D* | 255 | B | 355 | B* |
| 138 | D | 257 | C | 360 | C* |
| 141 | B | 258 | B | 361 | B* |
| 142 | B | 259 | B | 369 | D |
| 145 | B | 260 | B | 370 | B* |
| 186 | B* | 261 | B | 373 | B* |
| 190 | C | 265 | B | 374 | C* |
| 191 | B | 270 | A | 378 | A* |
| 194 | D | 273 | A | 383 | C* |
| 196 | D | 277 | A* | 384 | D* |
| 197 | C | 278 | A* | 385 | A* |
| 198 | D | 279 | B* | 386 | A* |
| 200 | D | 280 | B* | 387 | A* |
| 204 | D | 281 | A* | 388 | A* |
| 205 | B | 282 | A* | 389 | A* |
| 206 | B* | 291 | B | 390 | A* |
| 207 | C | 293 | B | 392 | A* |
| 208 | B | 296 | B | 394 | A* |
| 209 | A | 299 | D | 395 | D* |
| 211 | A | 303 | B | 396 | A* |
| 228 | D | 307 | B | 397 | C* |
| 229 | D | 308 | A | 398 | B* |
| 230 | D | 309 | A | 399 | A* |
| 231 | B | 311 | A | 400 | B* |
| 232 | B | 313 | D | 401 | A* |
| 233 | B | 319 | D | 403 | A* |
| 234 | A | | | 406 | A* |
| 236 | B | | | | |
| 238 | B | | | | |

* indicates that the EC₅₀ was performed in ACHN cells.
Otherwise EC₅₀ was performed in DAOY cells.

High-Content Imaging of pCdk1 Y¹⁵

For target engagement assessment of Wee1A kinase inhibition, high-content imaging of ACHN renal cell carcinoma cells was used for quantification of CDK1 phosphorylated on tyrosine 15 by immunofluorescence (“IF”). 20,000 cells per well were seeded in tissue culture treated 96 well plates and treated with compounds at concentrations ranging from 7 to 5,000 nM or from 0.3 to 200 nM for 4h. Cells were fixated for 15 minutes in 4% paraformaldehyde solution, washed in PBS and permeabilized using 0.2% Triton X-100. Blocking was performed for 1 h at RT using Blocker FL Fluorescent Blocking Buffer (Thermo Scientific), thereafter cells were incubated with primary antibody (rabbit anti-pCDK1 Y¹⁵, #4539, Cell Signaling Technology) at 4 °C ON. Alexa Fluor Plus 647 labelled goat anti-rabbit (A32733, Thermo Scientific) was used as secondary antibody. After washing in PBS, nuclei were stained with DAPI solution for 10 min at RT, protected from light. Cells were imaged using an ImageXpress Pico automated imaging system and CellReporter Xpress software (Molecular Devices). The percentage of cells with nuclear positivity for pCdk1 Y¹⁵ compared to DMSO control was determined for each well and drug dose response curves and EC₅₀ values were calculated and visualized using GraphPad Prism. Target engagement for Wee1A kinase can also be measured using the same AlphaLISA assay in OVCAR3 cells, an ovarian cancer cell line. NIH:OVCAR-3 (OVCAR3) cells are cultured in RPMI-1640 medium supplemented with 0.01 mg/ml insulin, 20% FBS, 1% Penicillin-Streptomycin and 10 mM HEPES buffer. 14,000 cells per well are seeded into 96 well tissue culture plates in full medium. Twenty-four hours post seeding, cells are treated with compound for 4 hours at concentrations ranging from 0.017 nM to 1000 nM.

Results are presented in Table 5, below, where compounds having an EC₅₀ less than or equal to 100 nM are represented as “A”; compounds having an EC₅₀ greater than 100 nM but less than or equal to 500 nM are represented as “B”; compounds having an EC₅₀ greater than 500 nM but less than or equal to 1000 nM are represented as “C”; and compounds having an EC₅₀ greater than 1000 nM are represented as “D”.

TABLE 5
CDK1 Tyr15 Phosphorylation EC₅₀
Values for Exemplary Compounds

| Compound | EC₅₀ | Compound | EC₅₀ | Compound | EC₅₀ |
|---|---|---|---|---|---|
| 100 | B | 107 | D | 114 | A |
| 101 | B | 108 | A | 115 | A |
| 102 | D | 109 | A | 116 | A |
| 103 | A | 110 | B | 117 | A |
| 104 | A | 111 | A | 118 | A |
| 105 | A | 112 | A | 119 | A |
| 106 | A | 113 | C | 120 | A |
| 121 | A | 172 | D | 229 | B |
| 122 | C | 174 | B | 230 | D |
| 123 | C | 175 | C | 231 | A |
| 124 | C | 176 | D | 232 | A |
| 125 | C | 177 | B | 233 | A |
| 126 | B | 178 | B | 234 | A |
| 127 | B | 179 | D | 235 | A |
| 128 | B | 180 | A | 236 | A |
| 129 | A | 181 | A | 237 | B |
| 130 | C | 182 | D | 238 | A |
| 131 | C | 183 | D | 239 | B |
| 132 | B | 184 | B | 240 | B |
| 133 | B | 185 | C | 24 | A |
| 134 | D | 186 | A | 242 | B |
| 135 | C | 187 | C | 243 | A |
| 136 | B | 188 | A | 244 | A |
| 137 | A | 189 | C | 245 | D |
| 138 | A | 190 | B | 246 | A |
| 139 | B | 191 | A | 247 | B |
| 140 | A | 192 | B | 248 | A |
| 141 | A | 193 | B | 249 | B |
| 142 | A | 194 | B | 250 | A |
| 143 | C | 195 | A | 25 | B |
| 144 | C | 196 | A | 252 | A |
| 145 | B | 197 | A | 253 | A |
| 146 | D | 198 | A | 254 | A |
| 147 | D | 199 | A | 255 | A |
| 148 | B | 200 | A | 256 | A |
| 149 | C | 201 | C | 257 | A |
| 150 | B | 202 | C | 258 | A |
| 151 | B | 204 | A | 259 | A |
| 152 | B | 205 | A | 260 | A |
| 153 | C | 206 | A | 26 | A |
| 154 | B | 207 | A | 262 | A |
| 155 | C | 208 | A | 263 | A |
| 156 | C | 209 | A | 264 | A |
| 157 | B | 211 | A | 265 | A |
| 158 | B | 219 | B | 266 | B |
| 159 | C | 220 | A | 267 | B |
| 160 | B | 221 | C | 268 | B |
| 161 | A | 222 | D | 269 | B |
| 165 | C | 223 | B | 270 | A |
| 166 | D | 224 | D | 271 | B |
| 168 | D | 225 | D | 272 | B |
| 169 | D | 226 | A | 273 | A |
| 170 | D | 227 | A | 274 | B |
| 171 | D | 228 | B | 275 | D |
| Compound | EC₅₀ | Compound | EC₅₀ | Compound | EC₅₀ |
| 276 | A | 323 | B | 368 | B |
| 277 | A | 324 | D | 369 | A |
| 278 | A | 326 | A | 370 | A |
| 279 | A | 327 | B | 371 | A |
| 280 | A | 328 | B | 372 | A |
| 281 | A | 329 | C | 373 | A |
| 282 | A | 330 | B | 374 | A |
| 284 | B | 331 | A | 375 | A |
| 285 | D | 332 | A | 376 | A |
| 286 | D | 333 | A | 377 | A |
| 287 | A | 334 | A | 378 | A |
| 288 | A | 335 | A | 379 | B |
| 289 | A | 336 | A | 380 | B |
| 290 | B | 337 | A | 381 | A |
| 291 | A | 338 | D | 382 | A |
| 292 | A | 339 | D | 384 | A |
| 293 | A | 340 | D | 385 | A |
| 296 | A | 341 | B | 386 | A |
| 297 | B | 342 | B | 387 | A |
| 298 | B | 343 | B | 388 | A |
| 299 | C | 344 | B | 389 | A |
| 300 | B | 345 | C | 390 | A |
| 301 | A | 346 | A | 391 | B |
| 302 | C | 347 | A | 392 | A |
| 303 | A | 348 | A | 393 | B |
| 304 | A | 349 | A | 394 | A |
| 305 | A | 350 | B | 395 | A |
| 306 | B | 351 | A | 396 | A |
| 307 | A | 352 | A | 397 | A |
| 308 | A | 353 | A | 398 | A |
| 309 | A | 354 | A | 399 | A |
| 310 | B | 355 | A | 400 | A |
| 311 | A | 356 | A | 401 | A |
| 312 | A | 357 | A | 402 | A |
| 313 | A | 358 | A | 403 | A |
| 314 | A | 359 | A | 404 | A |
| 315 | C | 360 | A | 405 | A |
| 316 | A | 361 | A | 406 | A |
| 317 | B | 362 | B | 407 | A |
| 318 | D | 363 | B | 408 | A |
| 319 | B | 364 | A | 409 | A |
| 320 | A | 365 | A | | |
| 321 | A | 366 | C | | |
| 322 | A | 367 | A | | |

### Cell Titer Glo Viability Assay

ACHN cells, DAOY cells, or A427 cells were seeded at a density of 400, 250, or 500 cells per well respectively, in tissue culture treated 384 well plates (Corning Costar). After overnight incubation, cells were treated with drugs at concentrations ranging from 17 to 10 000 nM. Viability compared to untreated control was assessed at 144 h using Cell Titer Glo assay 2.0 (Promega). Results are presented in Table 6, below, where compounds having an absolute EC₅₀ less than or equal to 100 nM are represented as “A”; compounds having an BC₅₀ greater than 100 nM but less than or equal to 500 nM are represented as “B”; compounds having an EC₅₀ greater than 500 nM but less than or equal to 1000 nM are represented as “C”; and compounds having an EC₅₀ greater than 1000 nM are represented as “D”. “NT” means the compounds was not tested in the indicated cells.

TABLE 6
Viability Data in Different Cell Lines for Exemplary Compounds

| | ACHN | DAOY | A427 |
|---|---|---|---|
| | viability | viability | viability |
| Cpd. | EC₅₀ | EC₅₀ | EC₅₀ |
| 100 | B | NT | NT |
| 102 | D | NT | NT |
| 103 | B | NT | NT |
| 104 | A | NT | NT |
| 105 | A | NT | NT |
| 106 | A | B | NT |
| 108 | B | NT | NT |
| 109 | A | A | A |
| 112 | A | B | NT |
| 113 | D | NT | NT |
| 114 | A | A | B |
| 116 | B | NT | NT |
| 118 | A | NT | NT |
| 119 | A | NT | NT |
| 121 | A | A | NT |
| 127 | C | NT | NT |
| 129 | A | NT | NT |
| 137 | A | NT | A |
| 138 | A | NT | NT |
| 139 | B | NT | NT |
| 140 | A | NT | NT |
| 141 | B | NT | NT |
| 142 | A | B | B |
| 145 | B | NT | NT |
| 157 | B | NT | NT |
| 161 | A | NT | B |
| 172 | D | NT | NT |
| 180 | A | NT | NT |
| 181 | A | NT | NT |
| 186 | A | A | NT |
| 188 | A | NT | NT |
| 190 | B | NT | NT |
| 191 | A | A | NT |
| 195 | A | NT | NT |
| 196 | A | NT | NT |
| 197 | A | A | NT |
| 198 | A | NT | NT |
| 199 | B | NT | NT |
| 200 | A | NT | NT |
| 204 | A | NT | NT |
| 205 | A | B | NT |
| 206 | A | NT | NT |
| 207 | A | A | NT |
| 208 | A | NT | NT |
| 209 | A | A | NT |
| 211 | A | A | A |
| 220 | A | NT | NT |
| 226 | B | NT | NT |
| 227 | A | NT | NT |
| 229 | A | NT | NT |
| 231 | A | A | NT |
| 233 | A | A | A |
| 234 | A | A | A |
| 235 | A | NT | NT |
| 236 | A | A | NT |
| 238 | A | NT | NT |
| 240 | B | NT | NT |
| 241 | A | NT | NT |
| 242 | B | NT | NT |
| 243 | B | NT | NT |
| 244 | B | NT | NT |
| 246 | B | NT | NT |
| 248 | A | A | NT |
| 250 | A | NT | NT |
| 252 | B | NT | NT |
| 253 | A | NT | NT |
| 254 | A | NT | NT |
| 255 | A | NT | NT |
| 256 | A | NT | NT |
| 257 | A | NT | NT |
| 258 | A | NT | NT |
| 259 | A | NT | NT |
| 260 | A | NT | NT |
| 261 | A | NT | NT |
| 270 | A | NT | NT |
| 273 | A | NT | NT |
| 277 | A | NT | NT |
| 278 | A | NT | NT |
| 279 | A | NT | NT |
| 280 | A | NT | NT |
| 281 | A | NT | NT |
| 282 | A | NT | NT |
| 287 | B | NT | NT |
| 288 | B | NT | NT |
| 289 | B | NT | NT |
| 290 | B | NT | NT |
| 291 | A | A | A |
| 292 | A | NT | NT |
| 293 | B | NT | NT |
| 296 | A | A | A |
| 298 | B | NT | NT |
| 301 | A | NT | NT |
| 303 | A | B | NT |
| 304 | B | NT | NT |
| 305 | A | NT | NT |
| 306 | B | NT | NT |
| 307 | A | A | NT |
| 308 | A | A | NT |
| 309 | A | A | NT |
| 310 | B | NT | NT |
| 311 | A | A | NT |
| 312 | B | NT | NT |
| 313 | A | NT | NT |
| 314 | A | NT | NT |
| 316 | B | NT | NT |
| 317 | B | NT | NT |
| 318 | D | NT | NT |
| 319 | C | NT | NT |
| 320 | A | A | NT |
| 321 | A | NT | NT |
| 322 | A | NT | NT |
| 331 | B | NT | NT |
| 332 | B | NT | NT |
| 333 | A | NT | NT |
| 334 | A | NT | NT |
| 335 | A | A | NT |
| 336 | B | NT | NT |
| 346 | A | NT | NT |
| 354 | A | NT | NT |
| 360 | A | NT | NT |
| 361 | A | NT | NT |
| 373 | A | NT | NT |
| 377 | A | NT | NT |
| 378 | A | NT | NT |
| 384 | A | NT | NT |
| 385 | A | NT | NT |
| 386 | A | NT | NT |
| 387 | A | NT | NT |
| 388 | A | NT | NT |
| 390 | A | NT | NT |
| 392 | A | NT | NT |
| 394 | A | NT | NT |
| 396 | A | NT | NT |
| 397 | A | NT | NT |
| 398 | A | NT | NT |
| 400 | A | NT | NT |
| 403 | A | NT | NT |

### Example 270. Treatment of OVCAR-3 Xenografts in Mice

OVCAR-3 cells are an ovarian cancer cell line and a model system in which to study drug efficacy in ovarian cancer. Female balb/c nude mice are inoculated subcutaneously at the right flank with 10×10⁶ OVCAR-3 cells in 0.2 mL of Dulbecco's Phosphate-Buffered Saline with 50% Matrigel®. Animals are randomized into groups of 3-8 mice when average tumor volume reaches 150-200 mm³ and treated by oral gavage with either vehicle control or a compound disclosed herein at doses of between 7.5 mg/kg and 100 mg/kg and at various dosing frequency (5 days in a row on/2 days in a row off; 3 days on/4 days off; 2 days on/5 days off; and 1 day on/6 days off (i.e., once weekly)). Tumor volume and body weight are recorded twice per week. Tumor volume is measured with a caliper and calculated using the formula V=0.5a×b² where a and b are the long and short diameters of the tumor in mm, respectively. Anti-tumor activity is assessed using two parameters: tumor growth inhibition (TGI) and T/C. TGI was calculated for each treatment group using the formula: TGI (%)=[1−(Tᵢ−T₀)/(Vᵢ−V₀)]×100], where Tᵢ is the average tumor volume of a treatment group on a given day, T₀ is the average tumor volume of the treatment group on the day of treatment start, Vᵢ is the average tumor volume of the vehicle control group on the same day as Tᵢ, and V₀ is the average tumor volume of the vehicle group on the day of treatment start. T/C (%) is calculated by dividing the mean tumor volume of the treated group on a given day by the mean tumor volume of the vehicle control group on the same day, multiplied by 100.

The relevant teachings of all patents, published applications and references cited herein are incorporated by reference in their entirety.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound having structural formula AA: 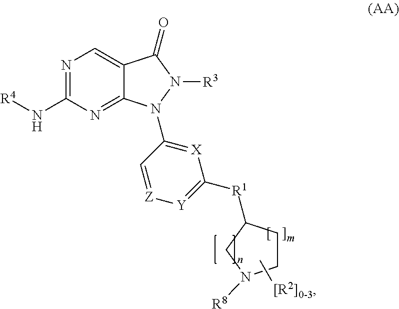
or a solvate, enantiomer, tautomer, or diastereomer thereof, or a pharmaceutically acceptable salt of any of the foregoing, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged or fused C₃-C₅ cycloalkyl ring, or one R² and R⁸ are optionally taken together to form a bridged or fused 3-5 membered ring;
R³ is —C₁-C₃ alkyl or —CH₂—CH═CH₂;
R⁴ is 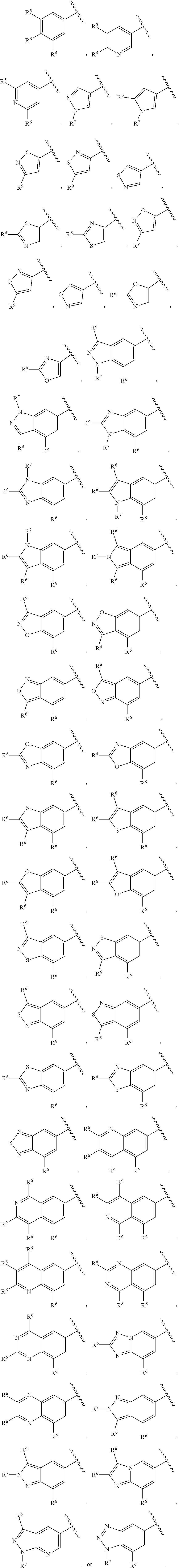
wherein “ [symbol] ” represents a point of attachment of R⁴ to the compound;
each R⁵ and each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl, wherein no more than two R⁶ are other than hydrogen; wherein R⁵ and an R⁶ on an adjacent ring atom are optionally taken together to form a 4-7 membered saturated heterocyclic or cycloalkyl ring that is fused to R⁴;
R⁷ is hydrogen, —C₁-C₄ alkyl, —C₁-C₄ alkylene-O—C₁-C₄ alkyl, —C(O)—C₁-C₄ alkyl, or a C₃-C₆ cycloalkyl, wherein any C₁-C₄ alkyl or C₁-C₄ alkylene portion of R⁷ is optionally substituted with one or more substituents independently selected from halo and —CN;
R⁸ is hydrogen, —C₁-C₄ alkyl, or a 4-6 membered saturated heterocycle;
R⁹ is hydrogen, halo, —CN, —C₁-C₄ alkyl optionally substituted with halo, —O—C₁-C₄ alkyl optionally substituted with halo, or an optionally substituted phenyl;
m is 0, 1, 2, 3 or 4;
n is 0, 1, 2 or 3; and
m+n is 1, 2, 3, or 4,
wherein any hydrogen atom is optionally replaced with deuterium.

2. The compound of claim 1, wherein:
each of X, Y and Z is independently CH or N;
R¹ is —O—, —NH—, or —N(C₁-C₃ alkyl)-;
each R² is independently fluoro, —CN, unsubstituted C₁-C₅ alkyl, fluoro-substituted C₁-C₅ alkyl, or cyano-substituted C₁-C₅ alkyl, wherein two R² bound to the same carbon atom are optionally taken together to form a spiro-fused C₃-C₇ cycloalkyl ring, or two R² bound to different carbon atoms are optionally taken together to form a bridged C₃-C₅ cycloalkyl ring, or one R² and R⁸ are optionally taken together to form a bridged or fused 3-5 membered ring;
R³ is —C₁-C₃ alkyl or —CH₂—CH═CH₂;
R⁴ is 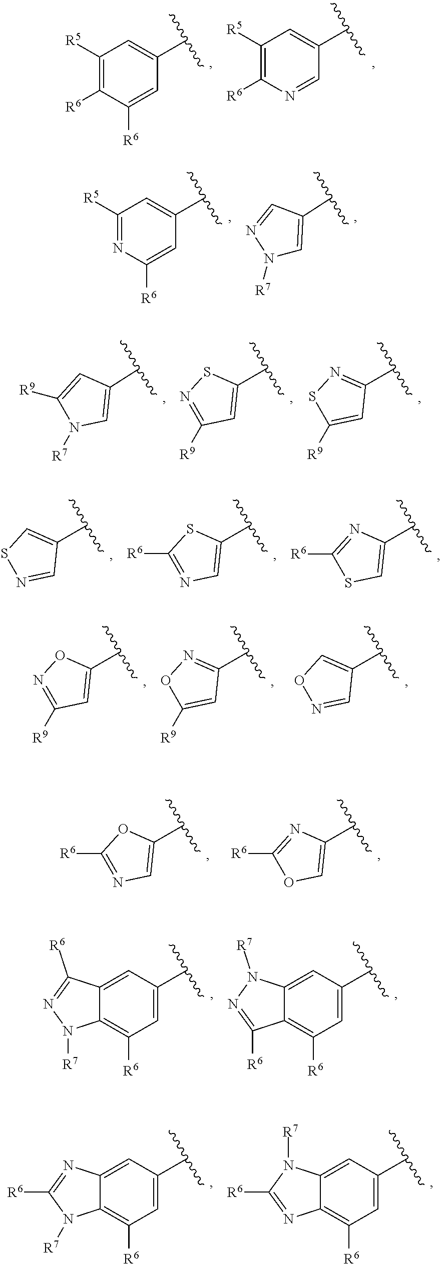 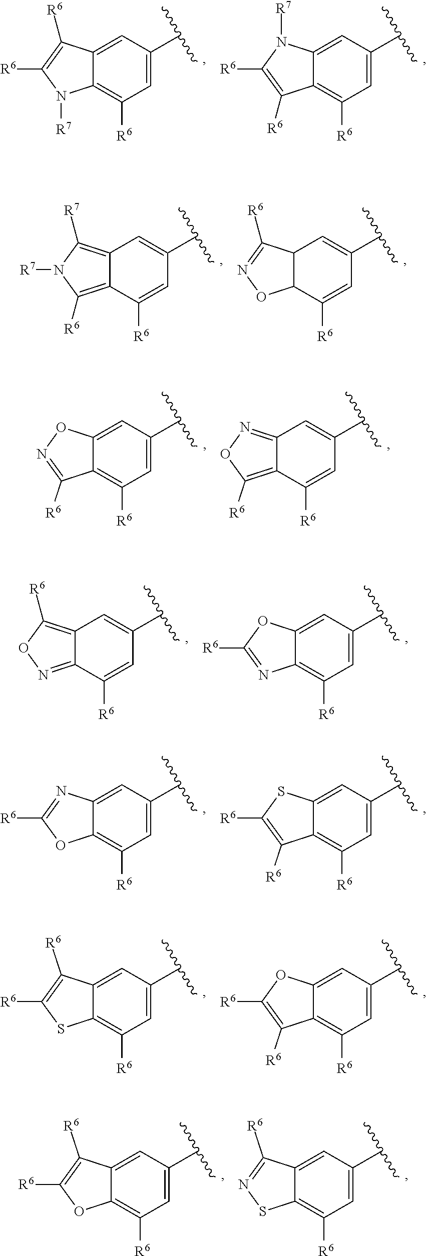 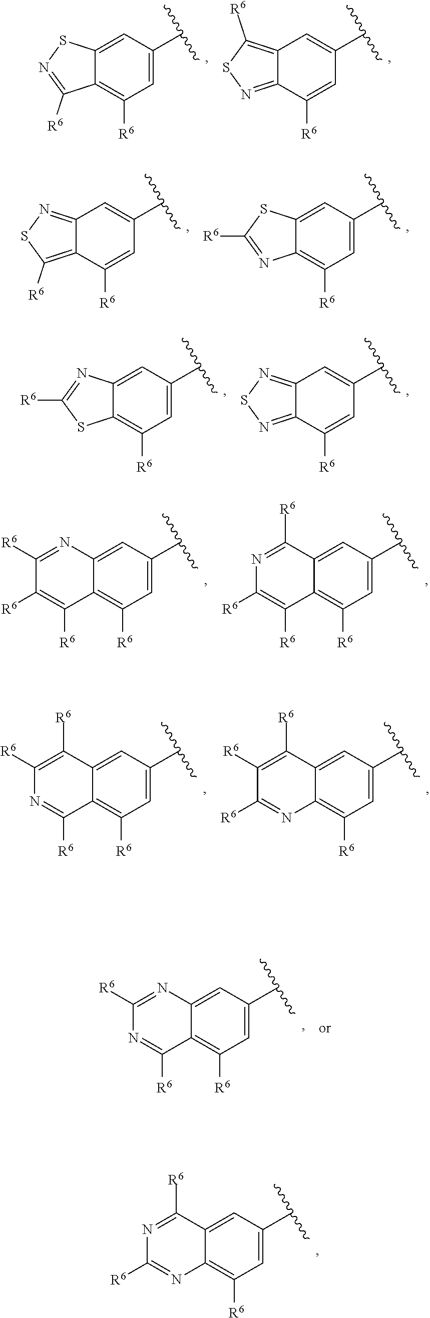
wherein “ [symbol] ” represents a point of attachment of R⁴ to the compound;
each R⁵ and each R⁶ is independently hydrogen, halo, —CN, —C₁-C₄ alkyl optionally substituted with halo, or —O—(C₁-C₄ alkyl) optionally substituted with halo, wherein no more than two R⁶ are other than hydrogen; wherein R⁵ and an R⁶ on an adjacent ring atom are taken together to form a 4-7 membered saturated heterocyclic or cycloalkyl ring that is fused to R⁴;
R⁷ is hydrogen, —C₁-C₄ alkyl, or —C₁-C₄ alkylene-O—C₁-C₄ alkyl, wherein any C₁-C₄ alkyl or C₁-C₄ alkylene portion of R⁷ is optionally substituted with one or more substituents independently selected from halo and —CN;
R⁸ is hydrogen or —C₁-C₄ alkyl;
R⁹ is hydrogen, halo, —CN, —C₁-C₄ alkyl optionally substituted with halo, —O—C₁-C₄ alkyl optionally substituted with halo, or an optionally substituted phenyl;
m is 0 or 1;
n is 0, 1, 2 or 3; and
m and n are not simultaneously 0.

3. The compound of claim 1 or 2, wherein:
each of X, Y and Z is CH.

4. The compound of claim 1 or 2, wherein:
X is N and each of Y and Z is CH.

5. The compound of claim 2, wherein:
each of X and Y is N and Z is CH.

6. The compound of any one of claims 1-5, wherein R⁴ is:

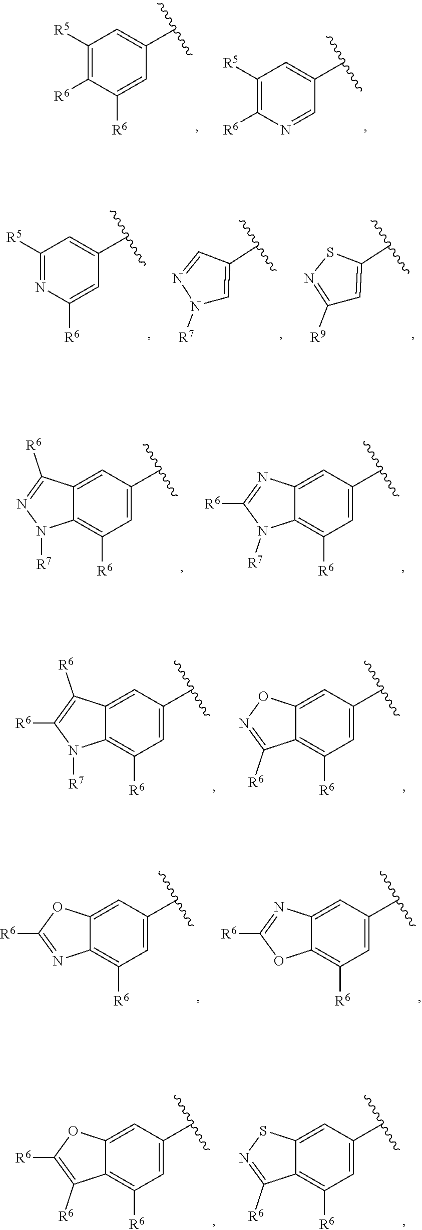

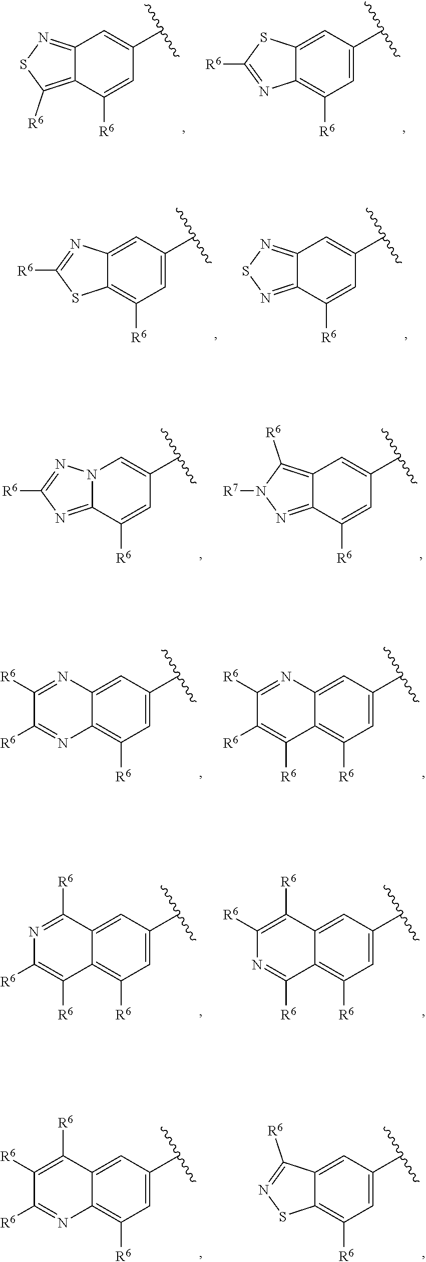

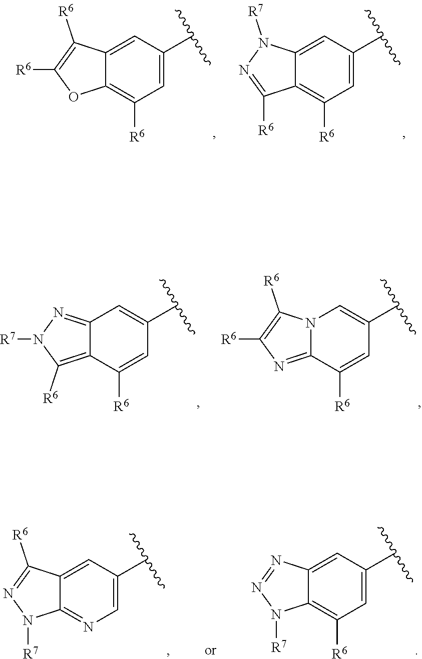

7. The compound of claim 6, wherein:
R⁴ is: 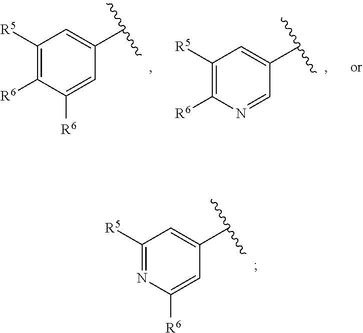
R⁵ is hydrogen, halo, —CN, C₁-C₄ alkyl optionally substituted with halo or —O—(C₁-C₄ alkyl) optionally substituted with halo; and
each R⁶ is independently hydrogen, halo, —CN, —C₁-C₄ alkyl optionally substituted with halo, —O—(C₁-C₄ alkyl) optionally substituted with halo, —O—(C₁-C₄ alkyl) substituted with C₃-C₆ cycloalkyl, an N-linked saturated 3-7 membered heterocyclyl, a 5-6 membered heteroaryl, or phenyl; or
R⁵ and R⁶ bound to an adjacent ring atom are taken together to form methylenedioxy.

8. The compound of any one of claims 1-7, wherein:
each R⁶ is independently hydrogen, fluoro, bromo, chloro, —CN, —CF₃, —CH₃, —OCH₃, —OCF₃, —OCH₂CF₃, —OCH₂CH₃, —OCH₂CH₂F, —OCH₂CH(CH₃)CH₃, cyclopropylmethoxy, morpholin-4-yl, thiomorpholin-4-yl, 1-methyl-1H-pyrazolyl, or phenyl optionally substituted with halo; and
each R⁵ is hydrogen, fluoro, chloro, —CN, —CF₃, —CH₃, or —OCH₃.

9. The compound of any one of claims 1-8, wherein no more than one R⁶ is other than hydrogen.

10. The compound of claim 6, wherein:
R⁴ is: 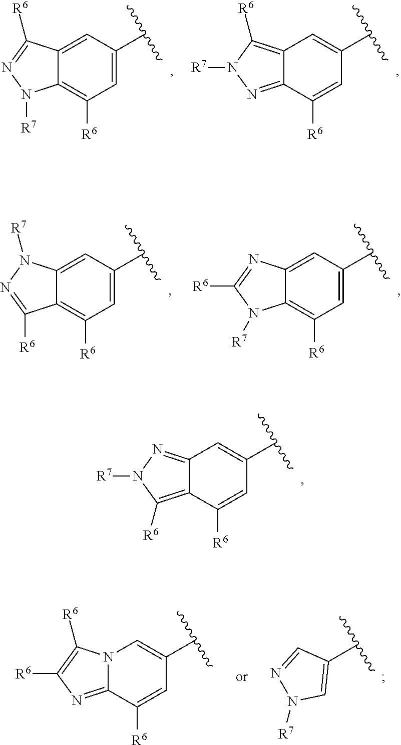
R⁶ is hydrogen, or chloro; and
R⁷ is hydrogen, —CH₃, —CH₂CH₃, —CH₂C(CH₃)₂F, —CH₂CH₂CF₃, —CH₂CH₂CH₃, —CH(CH₃)₂, —CH₂CH(CH₃)₂, —CH₂CH(CH₃)₂, —C(CH₃)₃, —CH₂CH₂CH₂F, —OCH₂CF₃, or cyclopropyl.

11. The compound of claim 6, wherein:
R⁴ is: 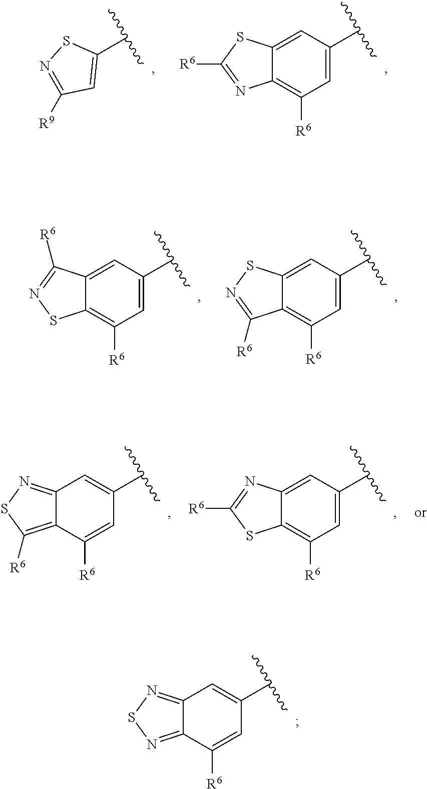
and
each R⁶ is hydrogen or —CH₃; and
R⁹ is optionally substituted phenyl.

12. The compound of claim 6, wherein:
R⁴ is: 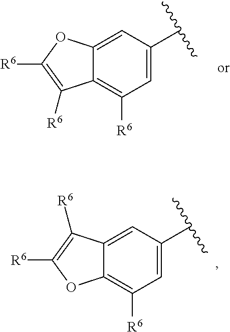
wherein each R⁶ is hydrogen.

13. The compound of claim 6, wherein R⁴ is 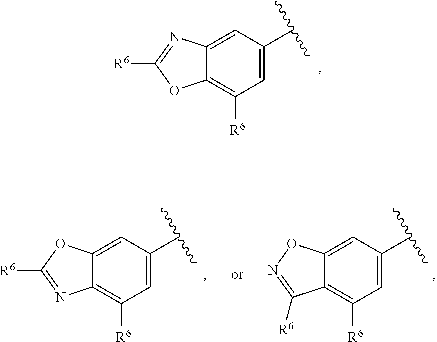
wherein each R⁶ is hydrogen or methyl.

14. The compound of claim 6, wherein R⁴ is 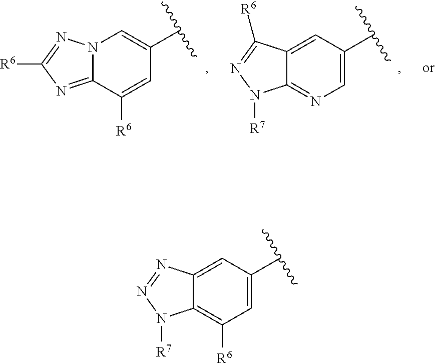
wherein each R⁶ is hydrogen; and R⁷, when present, is isopropyl.

15. The compound of claim 6, wherein R⁴ is 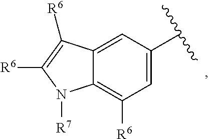
wherein R⁷ is methyl or —C(O)CH₃ and each R⁶ is hydrogen.

16. The compound of claim 6, wherein:
R⁴ is: 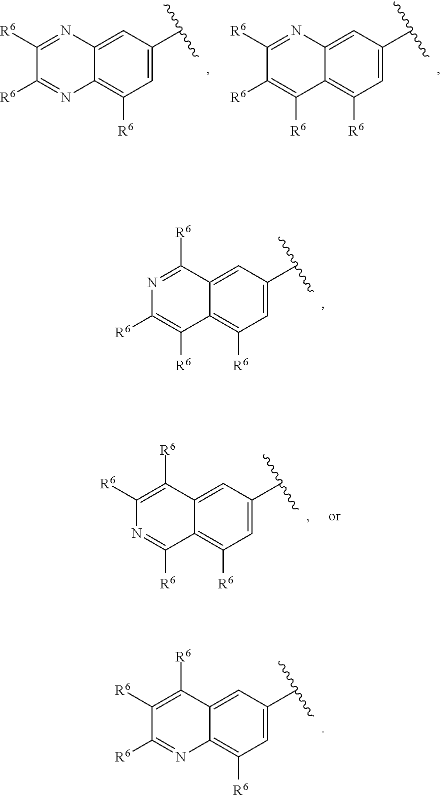
wherein each R⁶ is hydrogen.

17. The compound of claim 6, wherein R⁴ is phenyl, 3-methylphenyl, 3-chloro-5-bromophenyl, 3,4-dichlorophenyl, 4-bromophenyl, 4-chlorophenyl, 4-fluorophenyl, 4-(1-methylpiperidin-4-yl)-5-methylphenyl, 4-trifluoromethoxyphenyl, 3-methyl-4-trifluoromethoxyphenyl, 4-(2,2,2-trifluoroethan-1-yl)oxyphenyl, 3-methyl-4-(2,2,2-trifluoroethan-1-yl)oxyphenyl, 4-trifluoromethylphenyl, 3-methyl-4-chlorophenyl, 3-methyl-4-fluorophenyl, 3-methyl-5-fluorophenyl, 3-methyl-4-cyanophenyl, 3-methyl-4-methoxyphenyl, 3-methyl-4-(2,2,2-trifluoroethan-1-yloxy)phenyl, 3,4-methylenedioxyphenyl 4-(cyclopropylmethyloxy)phenyl, 4-(morpholin-4-yl)phenyl, 4-(thiomorpholin-4-yl)phenyl, 4-(2-methylpropan-1-yloxy)phenyl, 3-(1-methyl-1H-pyrazol-4-yl)phenyl, 4-(1-methyl-1H-pyrazol-4-yl)phenyl, 3-methyl-4-(morpholin-4-yl)phenyl, 4-(phenyl)phenyl, 3-bromophenyl, 3-cyanophenyl, 4-(2-fluoroethan-1-yloxy)phenyl, 4-cyanophenyl, 4-(1,1-dioxothiazinan-4-yl)phenyl, 3-(pyridin-3-yl)phenyl, 3-(pyrimidin-5-yl)phenyl, 3-(1H-imidazol-1-yl)phenyl, 3-(1H-pyrazol-1-yl)phenyl, 3-(d3-methyl)phenyl, 4-(3-fluoropropan-1-yloxy)phenyl, 4-ethoxyphenyl, 2-methylpyridin-4-yl, 6-methylpyridin-3-yl, 2,6-dimethylpyridin-4-yl, 2-trifluoromethyl-6-methylpyridin-4-yl, 2-trifluoromethylpyridin-4-yl, pyridin-3-yl, 2-methylpyridin-5-yl, 2-methoxypyridin-4-yl, 2-methoxypyridin-5-yl, 3-chloropyridin-5-yl, 3-fluoropyridin-5-yl, 3-cyanopyridin-5-yl, 3-methylpyridin-5-yl, 3-methoxypyridin-5-yl, 1-methyl-1H-pyrazol-4-yl, 1-propyl-1H-pyrazol-4yl, 1-isopropyl-1H-pyrazol-4-yl, 1-(2,2-dimethylethan-1-yl) pyrazol-4-yl, 1-(2-fluoro-2,2-dimethylethan-1-yl)-1H-pyrazol-4-yl, 1-(3,3,3-trifluoropropan-1-yl)-1H-pyrazol-4-yl, 1H-indazol-5-yl, 1-methyl-1H-indazol-5-yl, 1-ethyl-1H-indazol-5-yl, 1-propyl-1H-indazol-5-yl, 1-(3-fluoropropan-1-yl)-1H-indazol-5-yl, 1-isopropyl-1H-indazol-5-yl, 1-isobutyl-1H-indazol-5-yl, 1-cyclopropyl-1H-indazol-5-yl, 3-chloro-1H-indazol-5-yl, 3-methyl-1H-indazol-5-yl, 1,3-dimethyl-1H-indazol-5-yl, 1H-indazol-4-yl, 2-methyl-2H-indazol-5-yl, 2-ethyl-2H-indazol-5-yl, 3-phenylisothiazol-5-yl, benzofuran-6-yl, benzofuran-5-yl, 1-methyl-1H-indol-5-yl, 1-acetyl-1H-indol-5-yl, 1,2,4-triazolo[1,5-a]pyridin-6-yl, quinoxalin-6-yl, quinolin-6-yl, quinolin-7-yl, isoquinolin-6-yl, isoquinolin-7-yl, 2-methylbenzo[d]oxazol-5-yl, 2-methylbenzo[d]oxazol-6-yl, benzo[d]isoxazol-6-yl, benzo[d]thiazol-6-yl, 2-methylbenzo[d]thiazol-6-yl, 1-methyl-1H-benzo[d]imidazol-5-yl, benzo[d]thiazol-5-yl, 1-methyl-1H-indazol-6-yl, 2-methyl-2H-indazol-6-yl, imidazo[1,2-a]pyridin-6-yl, 2-(2-fluoroethoxy)pyridin-5-yl, 2-(1-butyl)-2H-indazol-5-yl, 1-isopropyl-1H-benzo[d]imidazol-5-yl, 1-isopropyl-1H-pyrazolo[3,4-b]pyridin-5-yl, or 1-isopropyl-1H-benzo[d][1,2,3]triazol-5-yl.

18. The compound of any one of claims 1-17, wherein the ring represented by the structure: 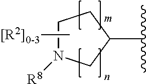
is selected from pyrrolidin-3-yl, 1-methylpyrrolidin-3-yl, 1-ethylpyrrolidin-3-yl, 1-isopropylpyrrolidin-3-yl, piperidin-4-yl, piperidin-3-yl, 1-methylpiperidin-4-yl, 1-methylpiperidin-3-yl, quinuclidin-3-yl, 8-methyl-8-azabicyclo[3.2.1]octan-3-yl, and 9-methyl-9-azabicyclo[3.3.1]nonan-3-yl.

19. The compound of any one of claims 1-17, wherein the ring represented by the structure: 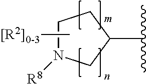
is selected from pyrrolidin-3-yl, 1-methylpyrrolidin-3-yl, 1-ethylpyrrolidin-3-yl, 1-isopropylpyrrolidin-3-yl, piperidin-4-yl, piperidin-3-yl, 1-methylpiperidin-4-yl, 1-methylpiperidin-3-yl, 1-(methyl-d3)piperidin-4-yl, azepan-4-yl, quinuclidin-3-yl, 8-methyl-8-azabicyclo[3.2.1]octan-3-yl, and 9-methyl-9-azabicyclo[3.3.1]nonan-3-yl.

20. The compound of any one of claims 1-19, wherein R³ is methyl, ethyl, n-propyl, or —CH₂—CH═CH₂.

21. The compound of claim 20, wherein R³ is —CH₂—CH═CH₂.

22. The compound of any one of claims 1-21, wherein R¹ is —O—, —N(CH₃)—, —N(CH₂CH₂CH₃)— or —NH—.

23. The compound of claim 22, wherein R¹ is —O—.

24. The compound of claim 1, wherein the compound is any one of the following compounds:

| # | Structure |
|---|---|
| 100 | 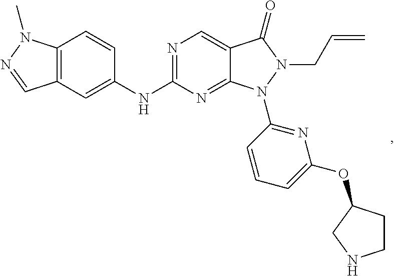 [SMILES: C=CCn1c(=O)c2cnc(Nc3ccc4c(cnn4C)c3)nc2n1-c1cccc(O[C@H]2CCNC2)n1] |
| 101 | 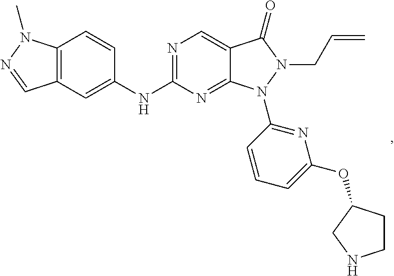 [SMILES: C=CCn1c(=O)c2cnc(Nc3ccc4c(cnn4C)c3)nc2n1-c1cccc(O[C@@H]2CCNC2)n1] |
| 102 | 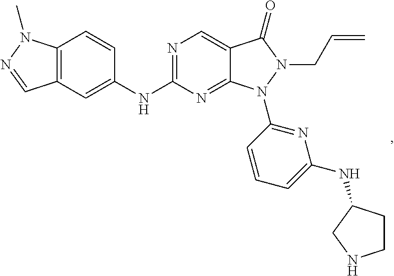 [SMILES: C=CCn1c(=O)c2cnc(Nc3ccc4c(cnn4C)c3)nc2n1-c1cccc(N[C@@H]2CCNC2)n1] |
| 103 | 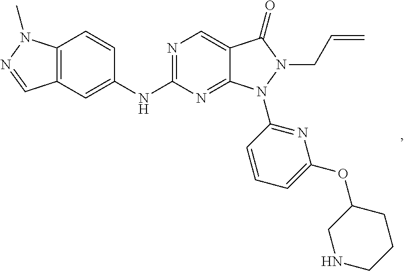 [SMILES: C=CCn1c(=O)c2cnc(Nc3ccc4c(cnn4C)c3)nc2n1-c1cccc(OC2CCCNC2)n1] |
| 104 | 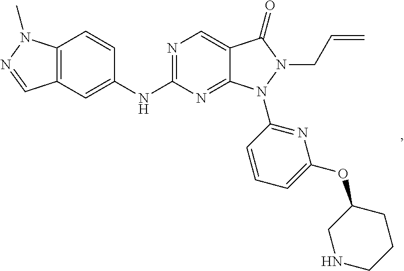 [SMILES: C=CCn1c(=O)c2cnc(Nc3ccc4c(cnn4C)c3)nc2n1-c1cccc(O[C@H]2CCCNC2)n1] |
| 105 | 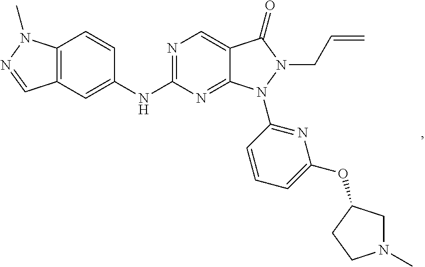 [SMILES: C=CCn1c(=O)c2cnc(Nc3ccc4c(cnn4C)c3)nc2n1-c1cccc(O[C@H]2CCN(C)C2)n1] |
| 106 | 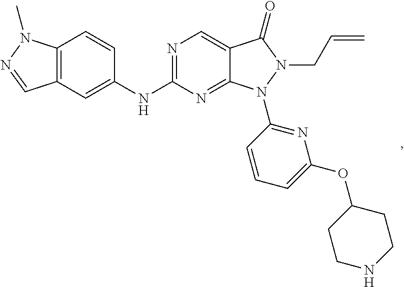 [SMILES: C=CCn1c(=O)c2cnc(Nc3ccc4c(cnn4C)c3)nc2n1-c1cccc(OC2CCNCC2)n1] |
| 107 | 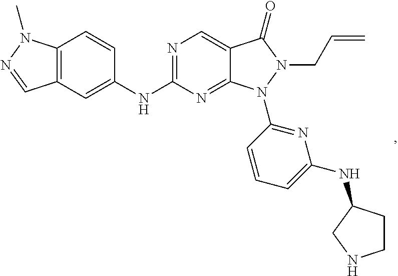 [SMILES: C=CCn1c(=O)c2cnc(Nc3ccc4c(cnn4C)c3)nc2n1-c1cccc(N[C@H]2CCNC2)n1] |
| 108 | 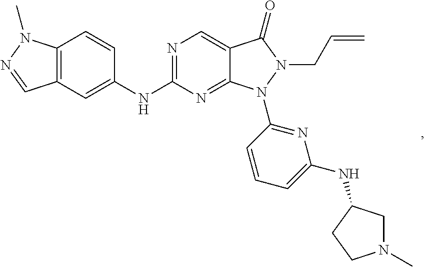 [SMILES: C=CCn1c(=O)c2cnc(Nc3ccc4c(cnn4C)c3)nc2n1-c1cccc(N[C@H]2CCN(C)C2)n1] |
| 109 | 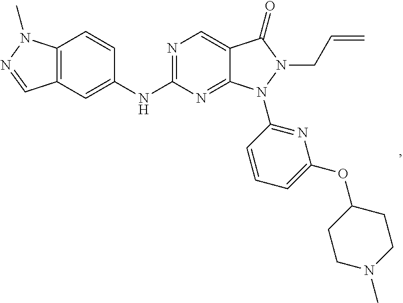 [SMILES: C=CCn1c(=O)c2cnc(Nc3ccc4c(cnn4C)c3)nc2n1-c1cccc(OC2CCN(C)CC2)n1] |
| 110 | 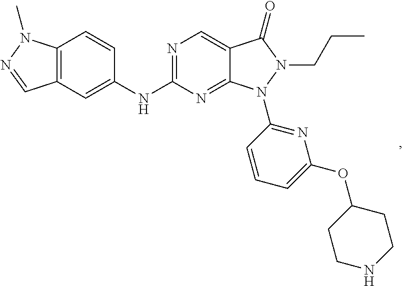 [SMILES: CCCn1c(=O)c2cnc(Nc3ccc4c(cnn4C)c3)nc2n1-c1cccc(OC2CCNCC2)n1] |
| 111 | 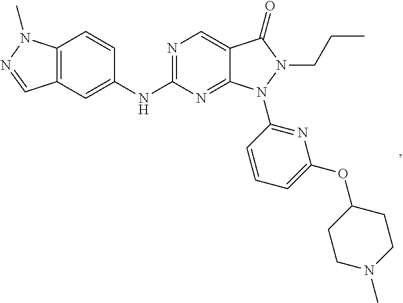 [SMILES: CCCn1c(=O)c2cnc(Nc3ccc4c(cnn4C)c3)nc2n1-c1cccc(OC2CCN(C)CC2)n1] |
| 112 | 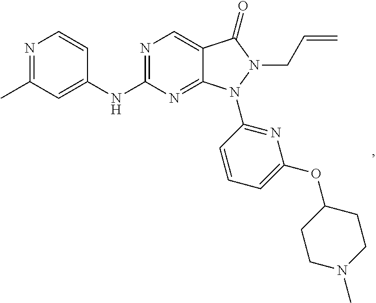 [SMILES: C=CCn1c(=O)c2cnc(Nc3ccnc(C)c3)nc2n1-c1cccc(OC2CCN(C)CC2)n1] |
| 113 | 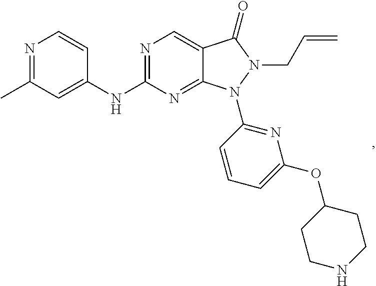 [SMILES: C=CCn1c(=O)c2cnc(Nc3ccnc(C)c3)nc2n1-c1cccc(OC2CCNCC2)n1] |
| 114 | 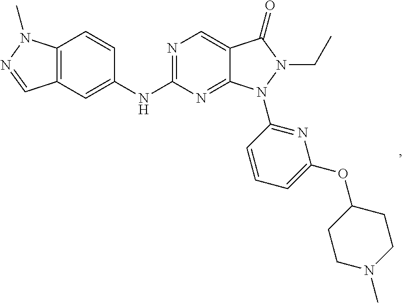 [SMILES: CCn1c(=O)c2cnc(Nc3ccc4c(cnn4C)c3)nc2n1-c1cccc(OC2CCN(C)CC2)n1] |
| 115 | 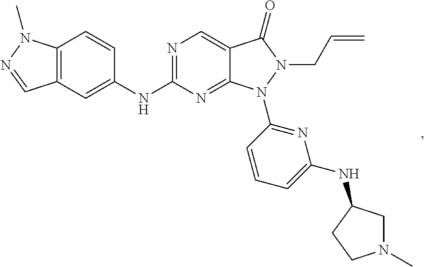 [SMILES: C=CCn1c(=O)c2cnc(Nc3ccc4c(cnn4C)c3)nc2n1-c1cccc(N[C@@H]2CCN(C)C2)n1] |
| 116 | 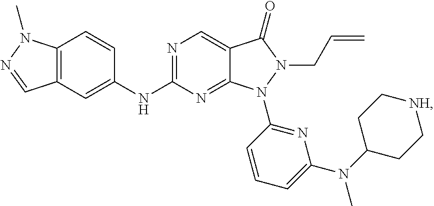 [SMILES: C=CCn1c(=O)c2cnc(Nc3ccc4c(cnn4C)c3)nc2n1-c1cccc(N(C)C2CCNCC2)n1] |
| 117 | 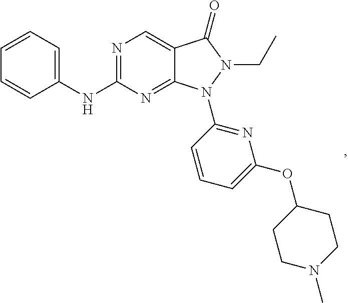 [SMILES: CCn1c(=O)c2cnc(Nc3ccccc3)nc2n1-c1cccc(OC2CCN(C)CC2)n1] |
| 118 | 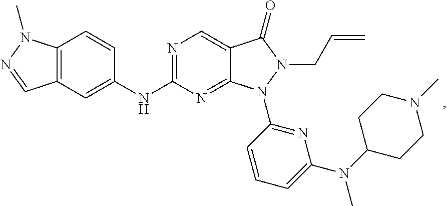 [SMILES: C=CCn1c(=O)c2cnc(Nc3ccc4c(cnn4C)c3)nc2n1-c1cccc(N(C)C2CCN(C)CC2)n1] |
| 119 | 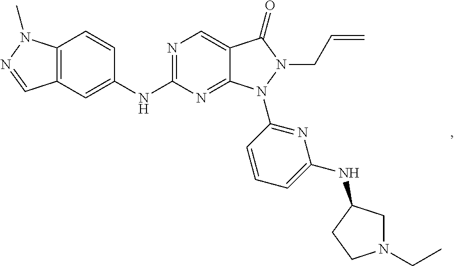 [SMILES: C=CCn1c(=O)c2cnc(Nc3ccc4c(cnn4C)c3)nc2n1-c1cccc(N[C@@H]2CCN(CC)C2)n1] |
| 120 | 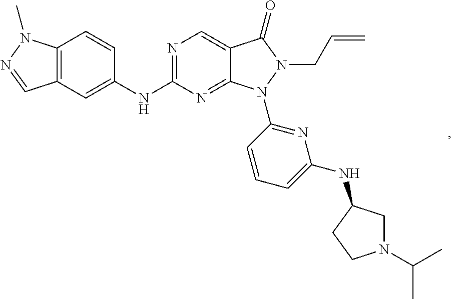 [SMILES: C=CCn1c(=O)c2cnc(Nc3ccc4c(cnn4C)c3)nc2n1-c1cccc(N[C@@H]2CCN(C(C)C)C2)n1] |
| 121 | 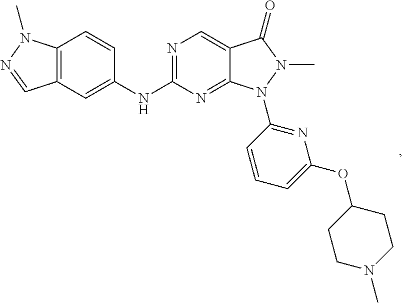 [SMILES: CN1CCC(Oc2cccc(-n3c4nc(Nc5ccc6c(cnn6C)c5)ncc4c(=O)n3C)n2)CC1] |
| 122 | 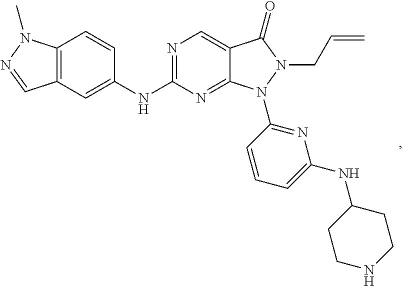 [SMILES: C=CCn1c(=O)c2cnc(Nc3ccc4c(cnn4C)c3)nc2n1-c1cccc(NC2CCNCC2)n1] |
| 123 | 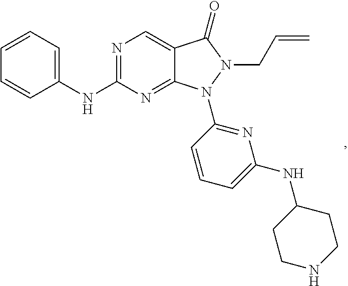 [SMILES: C=CCn1c(=O)c2cnc(Nc3ccccc3)nc2n1-c1cccc(NC2CCNCC2)n1] |
| 124 | 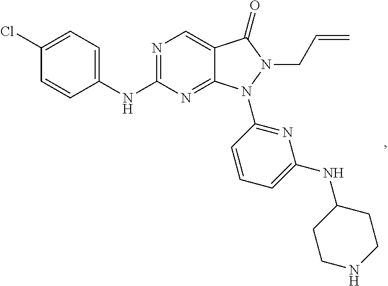 [SMILES: C=CCn1c(=O)c2cnc(Nc3ccc(Cl)cc3)nc2n1-c1cccc(NC2CCNCC2)n1] |
| 125 | 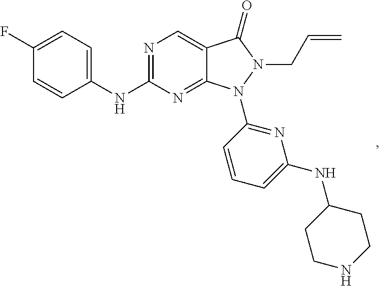 [SMILES: C=CCn1c(=O)c2cnc(Nc3ccc(F)cc3)nc2n1-c1cccc(NC2CCNCC2)n1] |
| 126 | 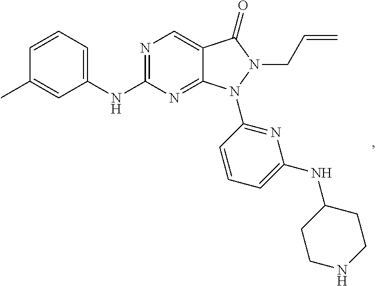 [SMILES: C=CCn1c(=O)c2cnc(Nc3cccc(C)c3)nc2n1-c1cccc(NC2CCNCC2)n1] |
| 127 | 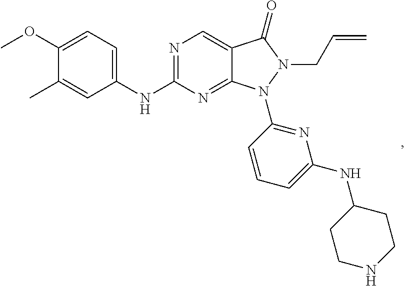 [SMILES: C=CCn1c(=O)c2cnc(Nc3ccc(OC)c(C)c3)nc2n1-c1cccc(NC2CCNCC2)n1] |
| 128 | 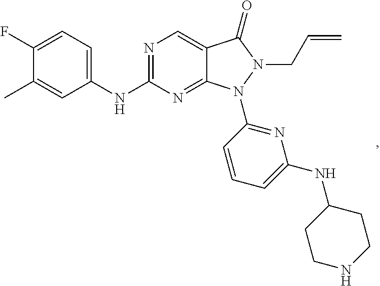 [SMILES: C=CCn1c(=O)c2cnc(Nc3ccc(F)c(C)c3)nc2n1-c1cccc(NC2CCNCC2)n1] |
| 129 | 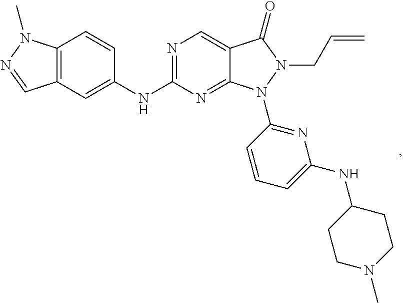 [SMILES: C=CCn1c(=O)c2cnc(Nc3ccc4c(cnn4C)c3)nc2n1-c1cccc(NC2CCN(C)CC2)n1] |
| 130 | 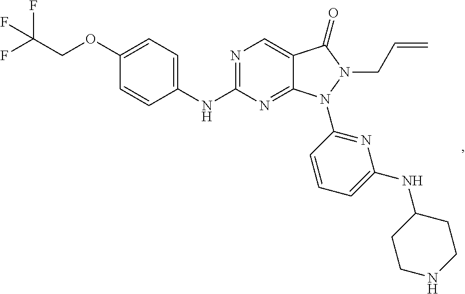 [SMILES: C=CCn1c(=O)c2cnc(Nc3ccc(OCC(F)(F)F)cc3)nc2n1-c1cccc(NC2CCNCC2)n1] |
| 131 | 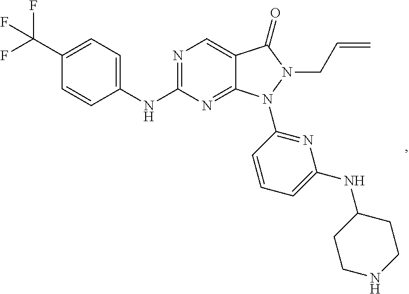 [SMILES: C=CCn1c(=O)c2cnc(Nc3ccc(C(F)(F)F)cc3)nc2n1-c1cccc(NC2CCNCC2)n1] |
| 132 | 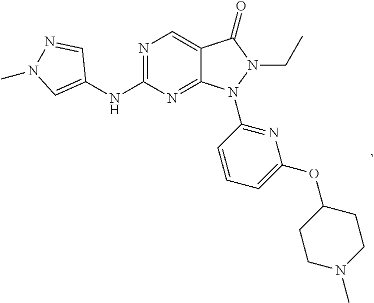 [SMILES: CCn1c(=O)c2cnc(Nc3cnn(C)c3)nc2n1-c1cccc(OC2CCN(C)CC2)n1] |
| 133 | 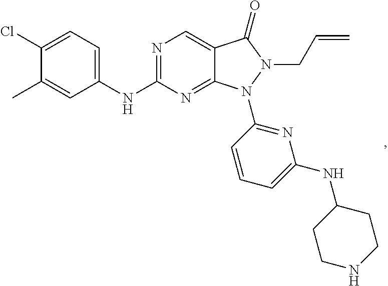 [SMILES: C=CCn1c(=O)c2cnc(Nc3ccc(Cl)c(C)c3)nc2n1-c1cccc(NC2CCNCC2)n1] |
| 134 | 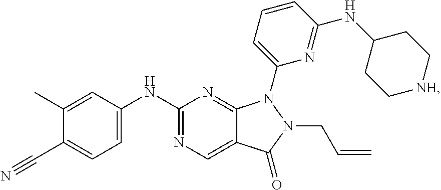 [SMILES: C=CCn1c(=O)c2cnc(Nc3ccc(C#N)c(C)c3)nc2n1-c1cccc(NC2CCNCC2)n1] |
| 135 | 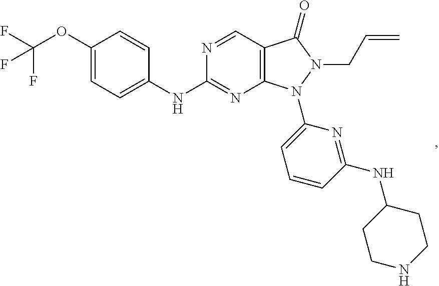 [SMILES: C=CCn1c(=O)c2cnc(Nc3ccc(OC(F)(F)F)cc3)nc2n1-c1cccc(NC2CCNCC2)n1] |
| 136 | 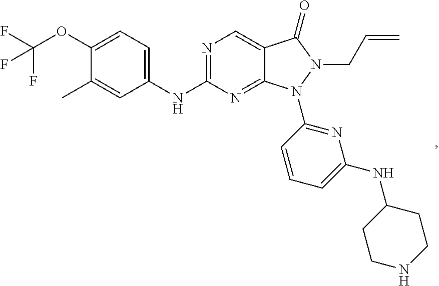 [SMILES: C=CCn1c(=O)c2cnc(Nc3ccc(OC(F)(F)F)c(C)c3)nc2n1-c1cccc(NC2CCNCC2)n1] |
| 137 | 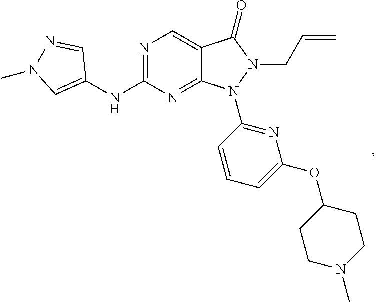 [SMILES: C=CCn1c(=O)c2cnc(Nc3cnn(C)c3)nc2n1-c1cccc(OC2CCN(C)CC2)n1] |
| 138 | 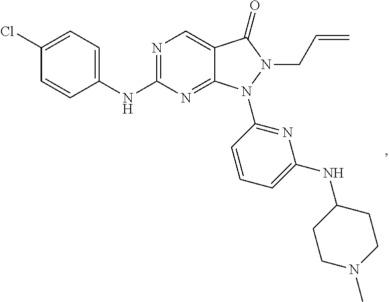 [SMILES: C=CCn1c(=O)c2cnc(Nc3ccc(Cl)cc3)nc2n1-c1cccc(NC2CCN(C)CC2)n1] |
| 139 | 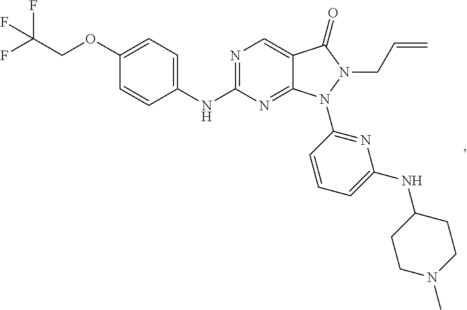 [SMILES: C=CCn1c(=O)c2cnc(Nc3ccc(OCC(F)(F)F)cc3)nc2n1-c1cccc(NC2CCN(C)CC2)n1] |
| 140 | 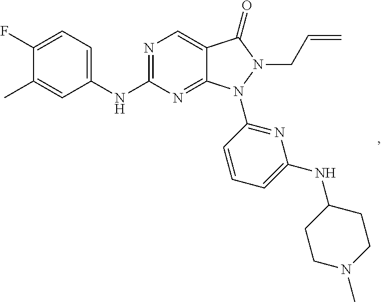 [SMILES: C=CCn1c(=O)c2cnc(Nc3ccc(F)c(C)c3)nc2n1-c1cccc(NC2CCN(C)CC2)n1] |
| 141 | 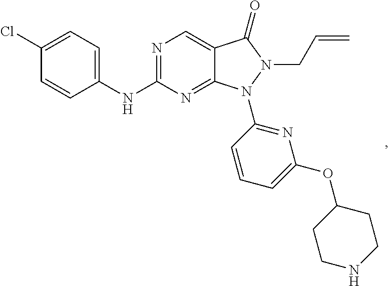 [SMILES: C=CCn1c(=O)c2cnc(Nc3ccc(Cl)cc3)nc2n1-c1cccc(OC2CCNCC2)n1] |
| 142 | 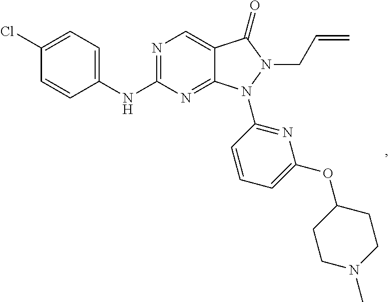 [SMILES: C=CCn1c(=O)c2cnc(Nc3ccc(Cl)cc3)nc2n1-c1cccc(OC2CCN(C)CC2)n1] |
| 143 | 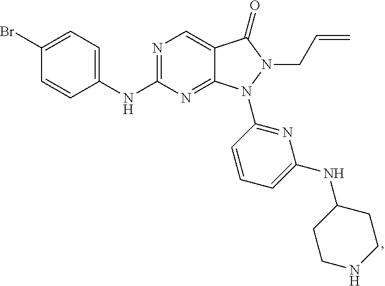 [SMILES: C=CCn1c(=O)c2cnc(Nc3ccc(Br)cc3)nc2n1-c1cccc(NC2CCNCC2)n1] |
| 144 | 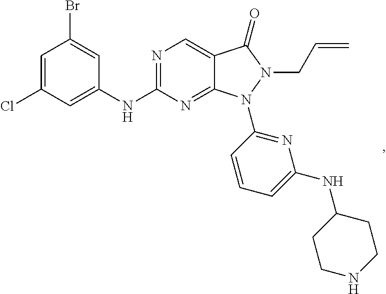 [SMILES: C=CCn1c(=O)c2cnc(Nc3cc(Cl)cc(Br)c3)nc2n1-c1cccc(NC2CCNCC2)n1] |
| 145 | 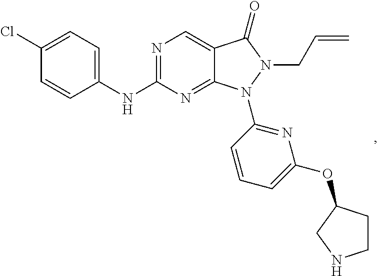 [SMILES: C=CCn1c(=O)c2cnc(Nc3ccc(Cl)cc3)nc2n1-c1cccc(O[C@H]2CCNC2)n1] |
| 146 | 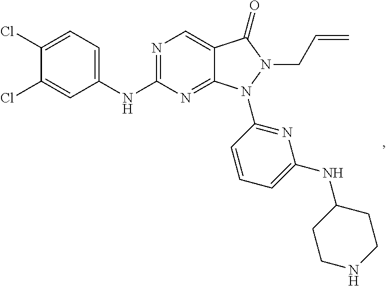 [SMILES: C=CCn1c(=O)c2cnc(Nc3ccc(Cl)c(Cl)c3)nc2n1-c1cccc(NC2CCNCC2)n1] |
| 147 | 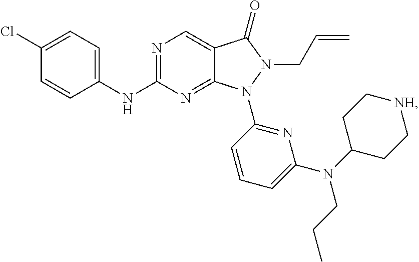 [SMILES: C=CCn1c(=O)c2cnc(Nc3ccc(Cl)cc3)nc2n1-c1cccc(N(CCC)C2CCNCC2)n1] |
| 148 | 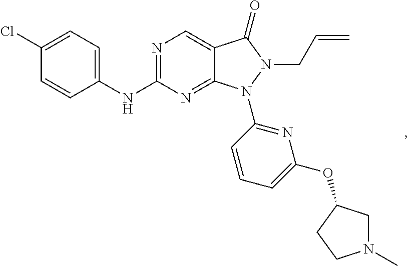 [SMILES: C=CCn1c(=O)c2cnc(Nc3ccc(Cl)cc3)nc2n1-c1cccc(O[C@H]2CCN(C)C2)n1] |
| 149 | 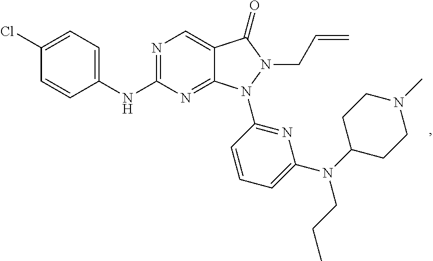 [SMILES: C=CCn1c(=O)c2cnc(Nc3ccc(Cl)cc3)nc2n1-c1cccc(N(CCC)C2CCN(C)CC2)n1] |
| 150 | 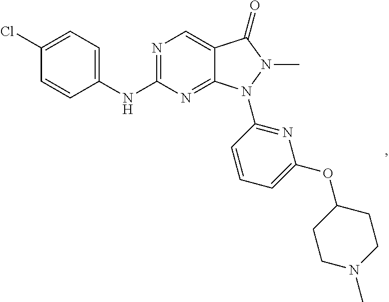 [SMILES: CN1CCC(Oc2cccc(-n3c4nc(Nc5ccc(Cl)cc5)ncc4c(=O)n3C)n2)CC1] |
| 151 | 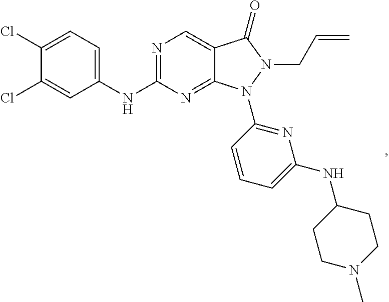 [SMILES: C=CCn1c(=O)c2cnc(Nc3ccc(Cl)c(Cl)c3)nc2n1-c1cccc(NC2CCN(C)CC2)n1] |
| 152 | 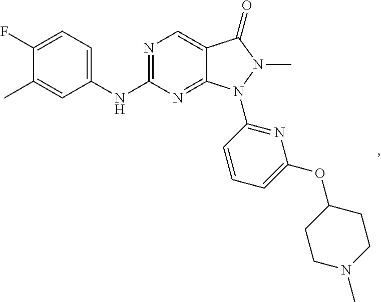 [SMILES: Cc1cc(Nc2ncc3c(=O)n(C)n(-c4cccc(OC5CCN(C)CC5)n4)c3n2)ccc1F] |
| 153 | 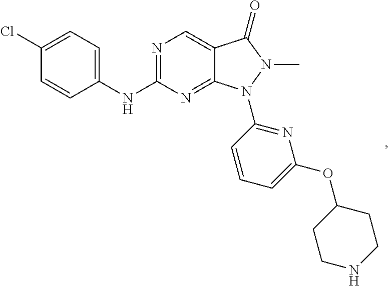 [SMILES: Cn1c(=O)c2cnc(Nc3ccc(Cl)cc3)nc2n1-c1cccc(OC2CCNCC2)n1] |
| 154 | 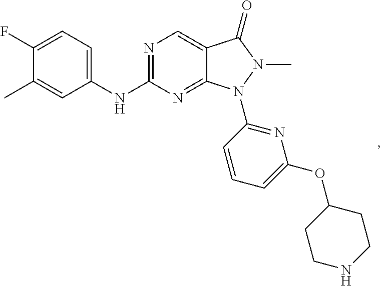 [SMILES: Cc1cc(Nc2ncc3c(=O)n(C)n(-c4cccc(OC5CCNCC5)n4)c3n2)ccc1F] |
| 155 | 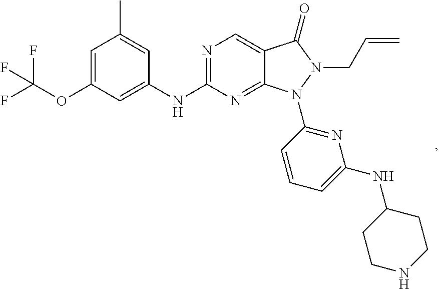 [SMILES: C=CCn1c(=O)c2cnc(Nc3cc(C)cc(OC(F)(F)F)c3)nc2n1-c1cccc(NC2CCNCC2)n1] |
| 156 | 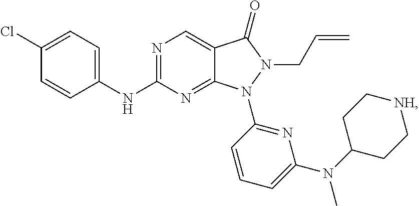 [SMILES: C=CCn1c(=O)c2cnc(Nc3ccc(Cl)cc3)nc2n1-c1cccc(N(C)C2CCNCC2)n1] |
| 157 | 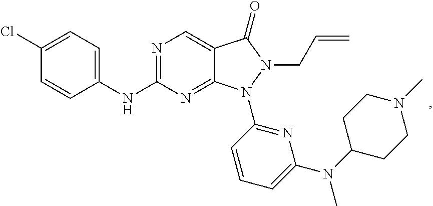 [SMILES: C=CCn1c(=O)c2cnc(Nc3ccc(Cl)cc3)nc2n1-c1cccc(N(C)C2CCN(C)CC2)n1] |
| 158 | 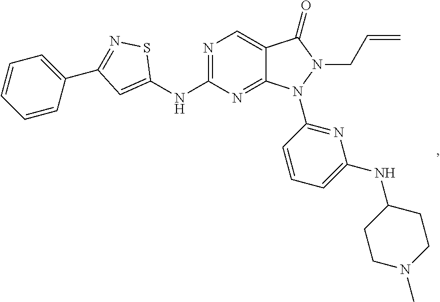 [SMILES: C=CCn1c(=O)c2cnc(Nc3cc(-c4ccccc4)ns3)nc2n1-c1cccc(NC2CCN(C)CC2)n1] |
| 159 | 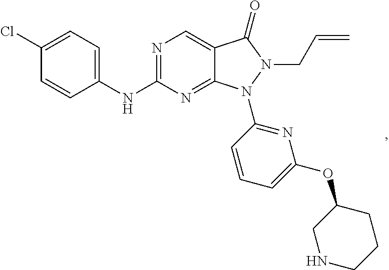 [SMILES: C=CCn1c(=O)c2cnc(Nc3ccc(Cl)cc3)nc2n1-c1cccc(O[C@H]2CCCNC2)n1] |
| 160 | 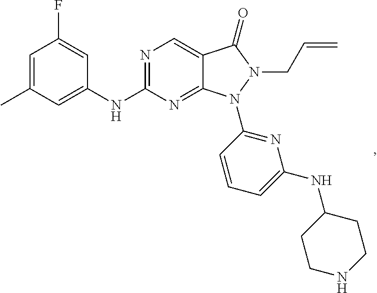 [SMILES: C=CCn1c(=O)c2cnc(Nc3cc(C)cc(F)c3)nc2n1-c1cccc(NC2CCNCC2)n1] |
| 161 | 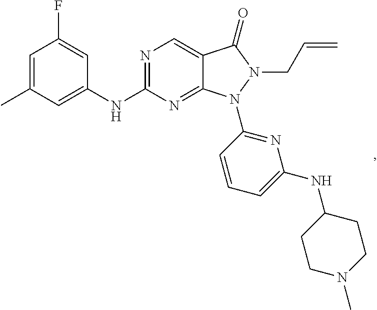 [SMILES: C=CCn1c(=O)c2cnc(Nc3cc(C)cc(F)c3)nc2n1-c1cccc(NC2CCN(C)CC2)n1] |
| 162 | 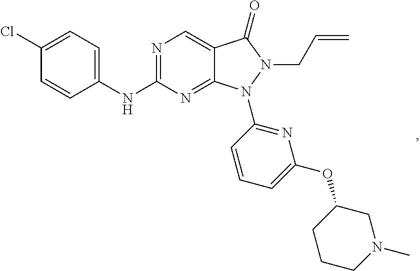 [SMILES: C=CCn1c(=O)c2cnc(Nc3ccc(Cl)cc3)nc2n1-c1cccc(O[C@H]2CCCN(C)C2)n1] |
| 163 | 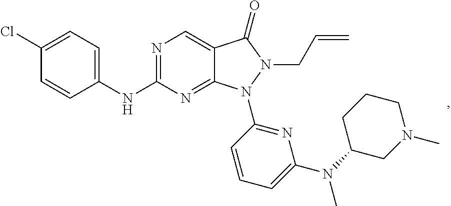 [SMILES: C=CCn1c(=O)c2cnc(Nc3ccc(Cl)cc3)nc2n1-c1cccc(N(C)[C@@H]2CCCN(C)C2)n1] |
| 164 | 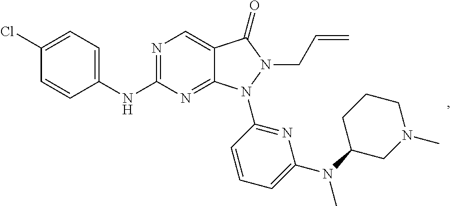 [SMILES: C=CCn1c(=O)c2cnc(Nc3ccc(Cl)cc3)nc2n1-c1cccc(N(C)[C@H]2CCCN(C)C2)n1] |
| 165 | 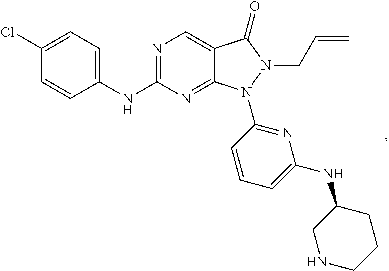 [SMILES: C=CCn1c(=O)c2cnc(Nc3ccc(Cl)cc3)nc2n1-c1cccc(N[C@H]2CCCNC2)n1] |
| 166 | 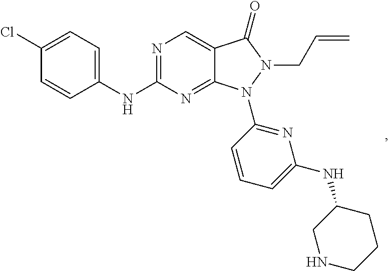 [SMILES: C=CCn1c(=O)c2cnc(Nc3ccc(Cl)cc3)nc2n1-c1cccc(N[C@@H]2CCCNC2)n1] |
| 167 | 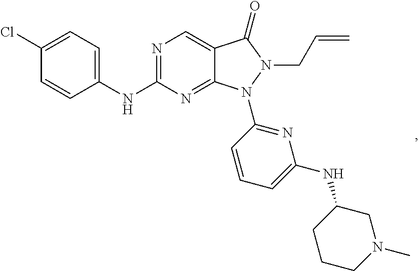 [SMILES: C=CCn1c(=O)c2cnc(Nc3ccc(Cl)cc3)nc2n1-c1cccc(N[C@H]2CCCN(C)C2)n1] |
| 168 | 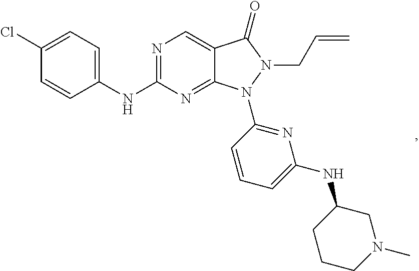 [SMILES: C=CCn1c(=O)c2cnc(Nc3ccc(Cl)cc3)nc2n1-c1cccc(N[C@@H]2CCCN(C)C2)n1] |
| 169 | 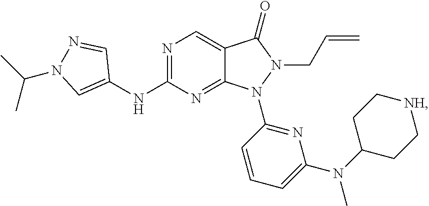 [SMILES: C=CCn1c(=O)c2cnc(Nc3cnn(C(C)C)c3)nc2n1-c1cccc(N(C)C2CCNCC2)n1] |
| 170 | 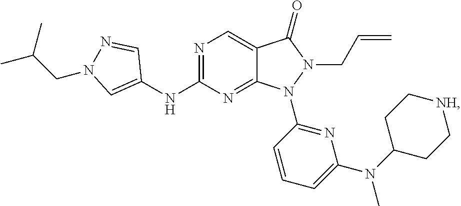 [SMILES: C=CCn1c(=O)c2cnc(Nc3cnn(CC(C)C)c3)nc2n1-c1cccc(N(C)C2CCNCC2)n1] |
| 171 | 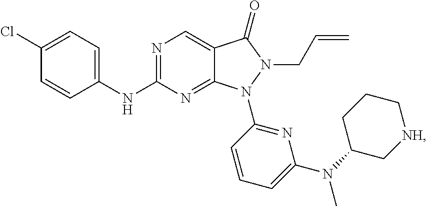 [SMILES: C=CCn1c(=O)c2cnc(Nc3ccc(Cl)cc3)nc2n1-c1cccc(N(C)[C@@H]2CCCNC2)n1] |
| 172 | 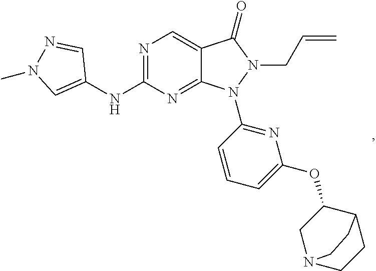 [SMILES: C=CCn1c(=O)c2cnc(Nc3cnn(C)c3)nc2n1-c1cccc(O[C@H]2CN3CCC2CC3)n1] |
| 173 | 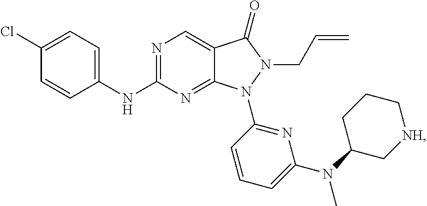 [SMILES: C=CCn1c(=O)c2cnc(Nc3ccc(Cl)cc3)nc2n1-c1cccc(N(C)[C@H]2CCCNC2)n1] |
| 174 | 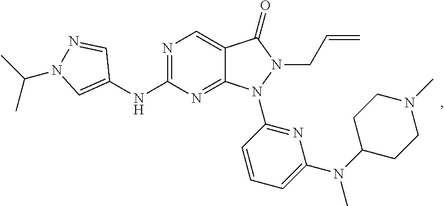 [SMILES: C=CCn1c(=O)c2cnc(Nc3cnn(C(C)C)c3)nc2n1-c1cccc(N(C)C2CCN(C)CC2)n1] |
| 175 | 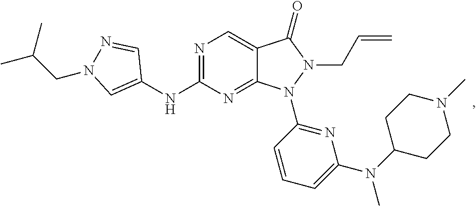 [SMILES: C=CCn1c(=O)c2cnc(Nc3cnn(CC(C)C)c3)nc2n1-c1cccc(N(C)C2CCN(C)CC2)n1] |
| 176 | 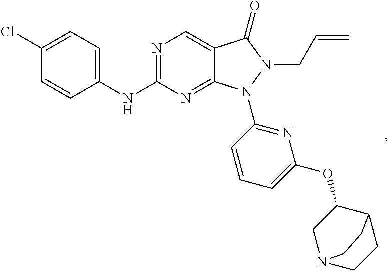 [SMILES: C=CCn1c(=O)c2cnc(Nc3ccc(Cl)cc3)nc2n1-c1cccc(O[C@H]2CN3CCC2CC3)n1] |
| 177 | 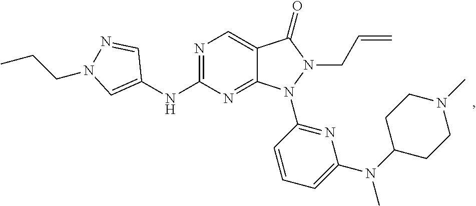 [SMILES: C=CCn1c(=O)c2cnc(Nc3cnn(CCC)c3)nc2n1-c1cccc(N(C)C2CCN(C)CC2)n1] |
| 178 | 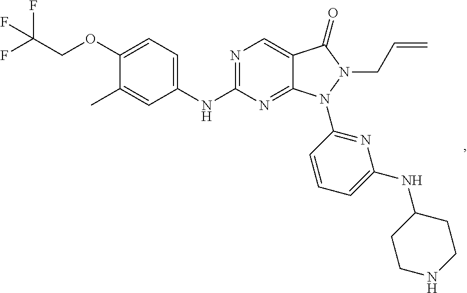 [SMILES: C=CCn1c(=O)c2cnc(Nc3ccc(OCC(F)(F)F)c(C)c3)nc2n1-c1cccc(NC2CCNCC2)n1] |
| 179 | 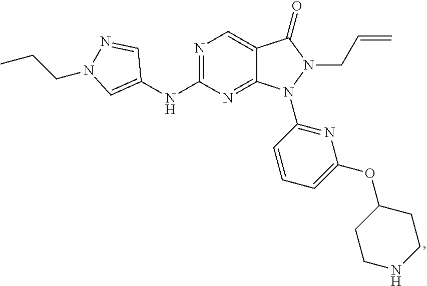 [SMILES: C=CCn1c(=O)c2cnc(Nc3cnn(CCC)c3)nc2n1-c1cccc(OC2CCNCC2)n1] |
| 180 | 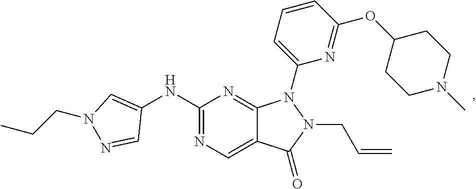 [SMILES: C=CCn1c(=O)c2cnc(Nc3cnn(CCC)c3)nc2n1-c1cccc(OC2CCN(C)CC2)n1] |
| 181 | 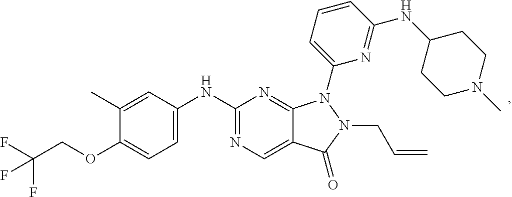 [SMILES: C=CCn1c(=O)c2cnc(Nc3ccc(OCC(F)(F)F)c(C)c3)nc2n1-c1cccc(NC2CCN(C)CC2)n1] |
| 182 | 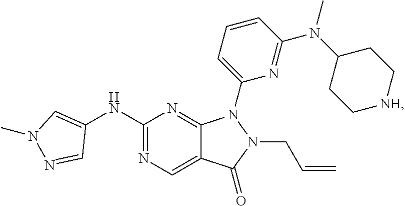 [SMILES: C=CCn1c(=O)c2cnc(Nc3cnn(C)c3)nc2n1-c1cccc(N(C)C2CCNCC2)n1] |
| 183 | 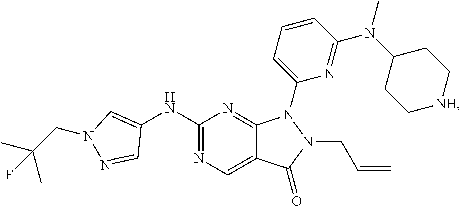 [SMILES: C=CCn1c(=O)c2cnc(Nc3cnn(CC(C)(C)F)c3)nc2n1-c1cccc(N(C)C2CCNCC2)n1] |
| 184 | 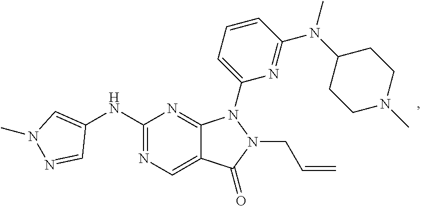 |
| 185 | 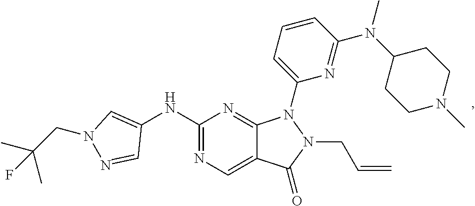 [SMILES: C=CCn1c(=O)c2cnc(Nc3cnn(CC(C)(C)F)c3)nc2n1-c1cccc(N(C)C2CCN(C)CC2)n1] |
| 186 | 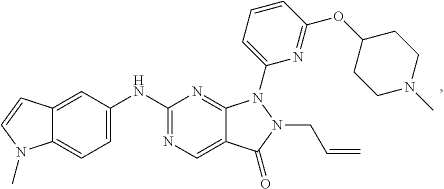 [SMILES: C=CCn1c(=O)c2cnc(Nc3ccc4c(ccn4C)c3)nc2n1-c1cccc(OC2CCN(C)CC2)n1] |
| 187 | 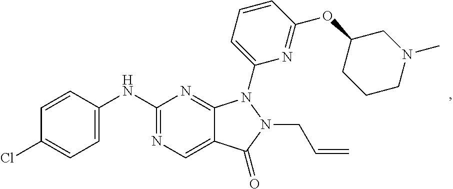 [SMILES: C=CCn1c(=O)c2cnc(Nc3ccc(Cl)cc3)nc2n1-c1cccc(O[C@@H]2CCCN(C)C2)n1] |
| 188 | 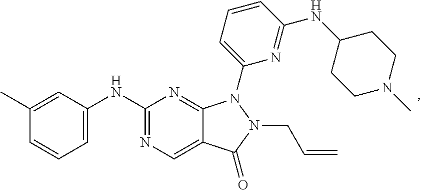 |
| 189 | 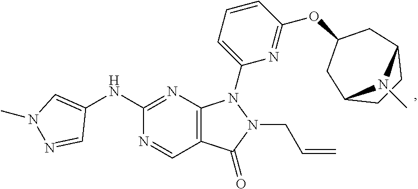 |
| 190 | 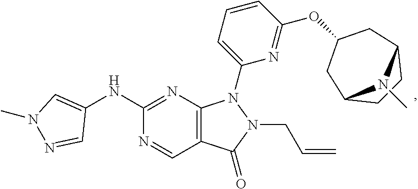 |
| 191 | 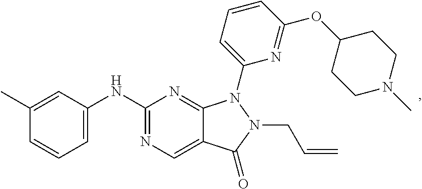 |
| 192 | 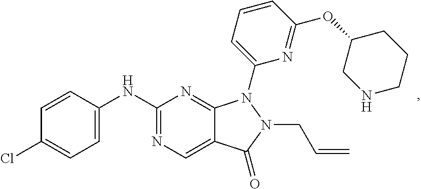 [SMILES: C=CCn1c(=O)c2cnc(Nc3ccc(Cl)cc3)nc2n1-c1cccc(O[C@@H]2CCCNC2)n1] |
| 193 | 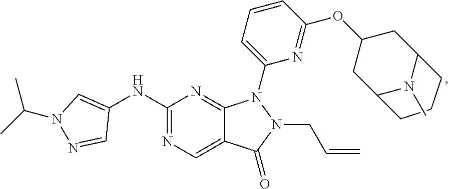 [SMILES: C=CCn1c(=O)c2cnc(Nc3cnn(C(C)C)c3)nc2n1-c1cccc(OC2CC3CCCC(C2)N3C)n1] |
| 194 | 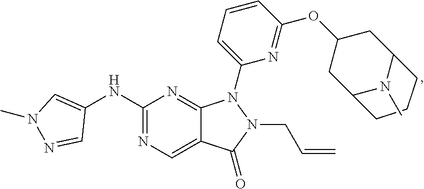 [SMILES: C=CCn1c(=O)c2cnc(Nc3cnn(C)c3)nc2n1-c1cccc(OC2CC3CCCC(C2)N3C)n1] |
| 195 | 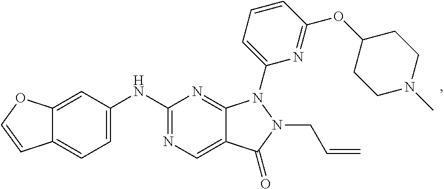 [SMILES: C=CCn1c(=O)c2cnc(Nc3ccc4ccoc4c3)nc2n1-c1cccc(OC2CCN(C)CC2)n1] |
| 196 | 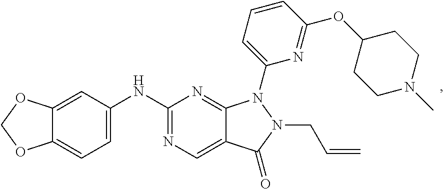 [SMILES: C=CCn1c(=O)c2cnc(Nc3ccc4c(c3)OCO4)nc2n1-c1cccc(OC2CCN(C)CC2)n1] |
| 197 | 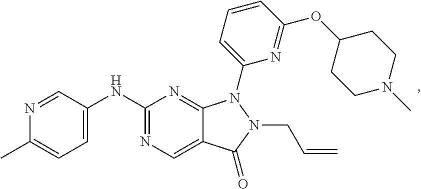 |
| 198 | 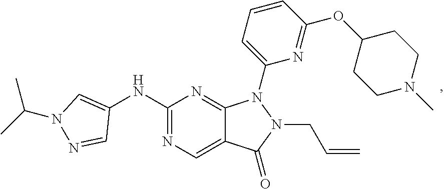 [SMILES: C=CCn1c(=O)c2cnc(Nc3cnn(C(C)C)c3)nc2n1-c1cccc(OC2CCN(C)CC2)n1] |
| 199 | 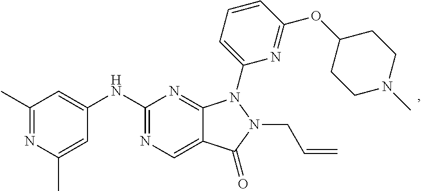 |
| 200 | 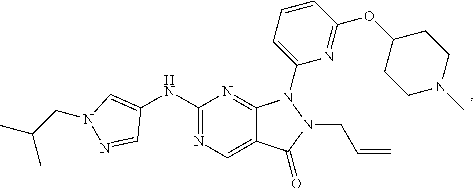 [SMILES: C=CCn1c(=O)c2cnc(Nc3cnn(CC(C)C)c3)nc2n1-c1cccc(OC2CCN(C)CC2)n1] |
| 201 | 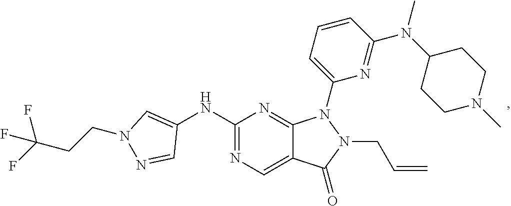 [SMILES: C=CCn1c(=O)c2cnc(Nc3cnn(CCC(F)(F)F)c3)nc2n1-c1cccc(N(C)C2CCN(C)CC2)n1] |
| 202 | 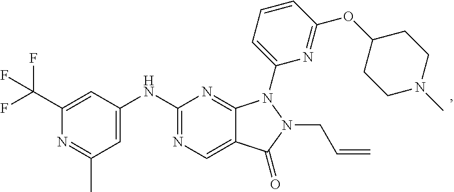 [SMILES: C=CCn1c(=O)c2cnc(Nc3cc(C)nc(C(F)(F)F)c3)nc2n1-c1cccc(OC2CCN(C)CC2)n1] |
| 204 | 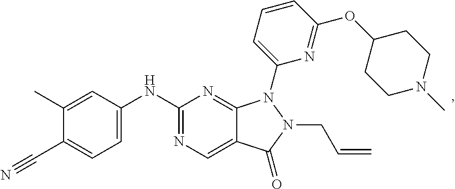 [SMILES: C=CCn1c(=O)c2cnc(Nc3ccc(C#N)c(C)c3)nc2n1-c1cccc(OC2CCN(C)CC2)n1] |
| 205 | 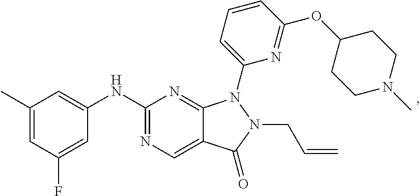 |
| 206 | 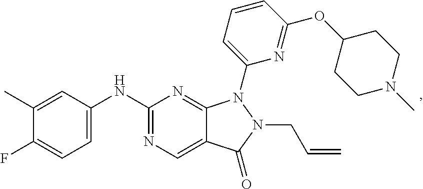 |
| 207 | 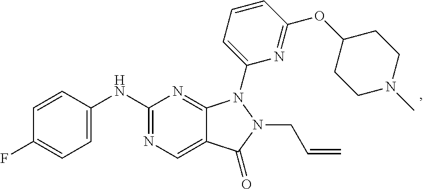 [SMILES: C=CCn1c(=O)c2cnc(Nc3ccc(F)cc3)nc2n1-c1cccc(OC2CCN(C)CC2)n1] |
| 208 | 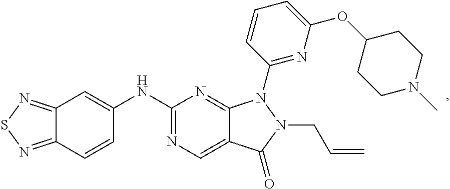 [SMILES: C=CCn1c(=O)c2cnc(Nc3ccc4nsnc4c3)nc2n1-c1cccc(OC2CCN(C)CC2)n1] |
| 209 | 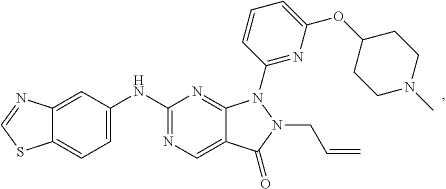 [SMILES: C=CCn1c(=O)c2cnc(Nc3ccc4scnc4c3)nc2n1-c1cccc(OC2CCN(C)CC2)n1] |
| 211 | 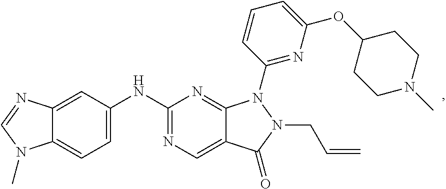 [SMILES: C=CCn1c(=O)c2cnc(Nc3ccc4c(c3)ncn4C)nc2n1-c1cccc(OC2CCN(C)CC2)n1] |
| 212 | 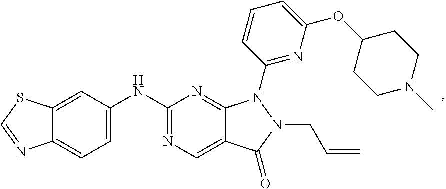 [SMILES: C=CCn1c(=O)c2cnc(Nc3ccc4ncsc4c3)nc2n1-c1cccc(OC2CCN(C)CC2)n1] |
| 214 | 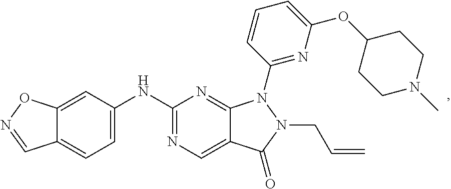 [SMILES: C=CCn1c(=O)c2cnc(Nc3ccc4cnoc4c3)nc2n1-c1cccc(OC2CCN(C)CC2)n1] |
| 217 | 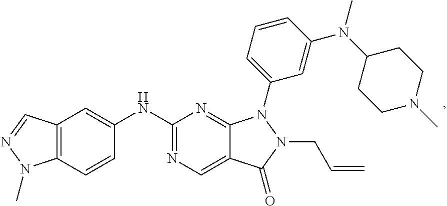 [SMILES: C=CCn1c(=O)c2cnc(Nc3ccc4c(cnn4C)c3)nc2n1-c1cccc(N(C)C2CCN(C)CC2)c1] |
| 218 | 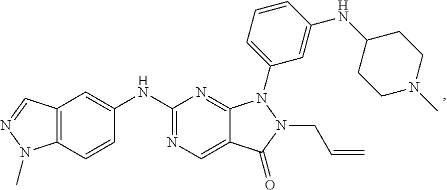 [SMILES: C=CCn1c(=O)c2cnc(Nc3ccc4c(cnn4C)c3)nc2n1-c1cccc(NC2CCN(C)CC2)c1] |
| 219 | 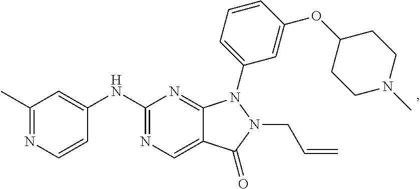 |
| 220 | 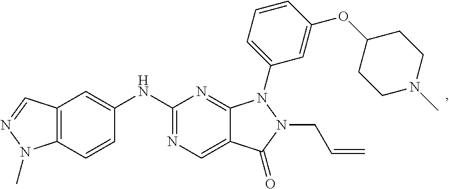 [SMILES: C=CCn1c(=O)c2cnc(Nc3ccc4c(cnn4C)c3)nc2n1-c1cccc(OC2CCN(C)CC2)c1] |
| 221 | 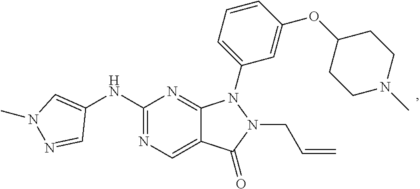 |
| 222 | 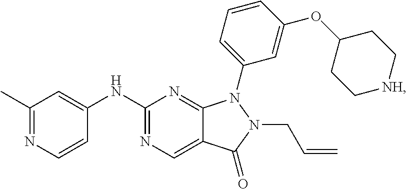 [SMILES: C=CCn1c(=O)c2cnc(Nc3ccnc(C)c3)nc2n1-c1cccc(OC2CCNCC2)c1] |
| 223 | 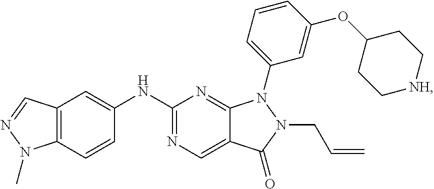 [SMILES: C=CCn1c(=O)c2cnc(Nc3ccc4c(cnn4C)c3)nc2n1-c1cccc(OC2CCNCC2)c1] |
| 224 | 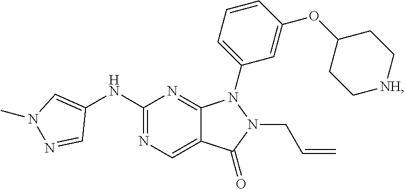 [SMILES: C=CCn1c(=O)c2cnc(Nc3cnn(C)c3)nc2n1-c1cccc(OC2CCNCC2)c1] |
| 225 | 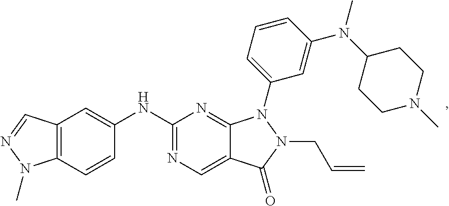 [SMILES: C=CCn1c(=O)c2cnc(Nc3ccc4c(cnn4C)c3)nc2n1-c1cccc(N(C)C2CCN(C)CC2)c1] |
| 226 | 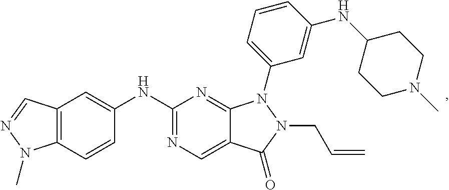 [SMILES: C=CCn1c(=O)c2cnc(Nc3ccc4c(cnn4C)c3)nc2n1-c1cccc(NC2CCN(C)CC2)c1] |
| 227 | 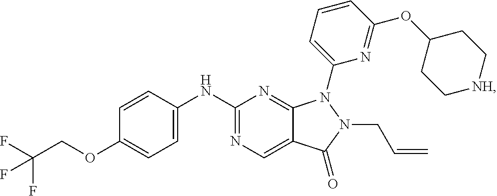 [SMILES: C=CCn1c(=O)c2cnc(Nc3ccc(OCC(F)(F)F)cc3)nc2n1-c1cccc(OC2CCNCC2)n1] |
| 228 | 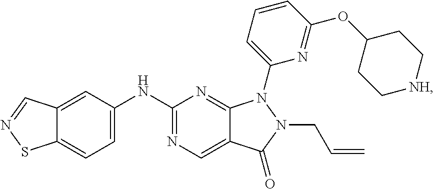 [SMILES: C=CCn1c(=O)c2cnc(Nc3ccc4sncc4c3)nc2n1-c1cccc(OC2CCNCC2)n1] |
| 229 | 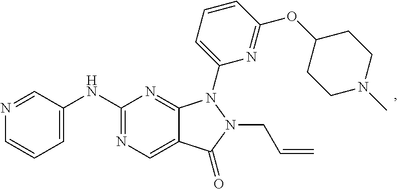 [SMILES: C=CCn1c(=O)c2cnc(Nc3cccnc3)nc2n1-c1cccc(OC2CCN(C)CC2)n1] |
| 230 | 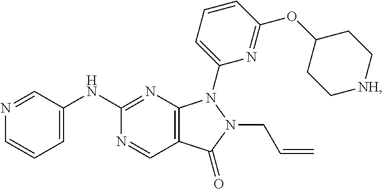 [SMILES: C=CCn1c(=O)c2cnc(Nc3cccnc3)nc2n1-c1cccc(OC2CCNCC2)n1] |
| 231 | 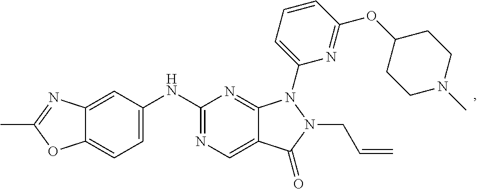 |
| 232 | 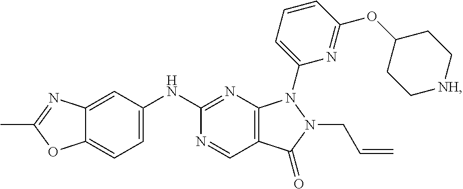 [SMILES: C=CCn1c(=O)c2cnc(Nc3ccc4oc(C)nc4c3)nc2n1-c1cccc(OC2CCNCC2)n1] |
| 233 | 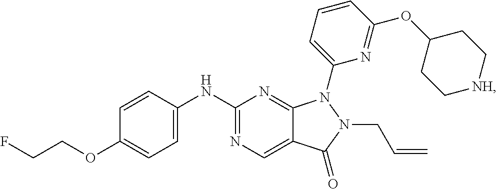 [SMILES: C=CCn1c(=O)c2cnc(Nc3ccc(OCCF)cc3)nc2n1-c1cccc(OC2CCNCC2)n1] |
| 234 | 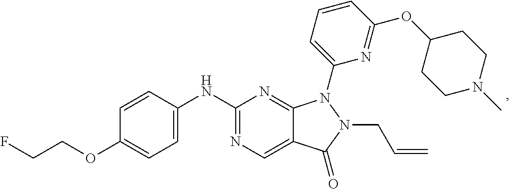 [SMILES: C=CCn1c(=O)c2cnc(Nc3ccc(OCCF)cc3)nc2n1-c1cccc(OC2CCN(C)CC2)n1] |
| 235 | 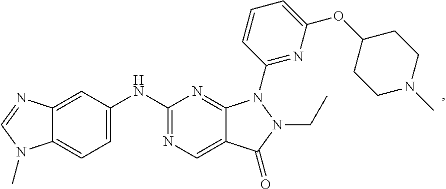 [SMILES: CCn1c(=O)c2cnc(Nc3ccc4c(c3)ncn4C)nc2n1-c1cccc(OC2CCN(C)CC2)n1] |
| 236 | 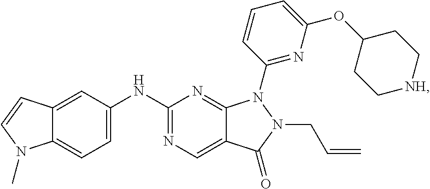 [SMILES: C=CCn1c(=O)c2cnc(Nc3ccc4c(ccn4C)c3)nc2n1-c1cccc(OC2CCNCC2)n1] |
| 237 | 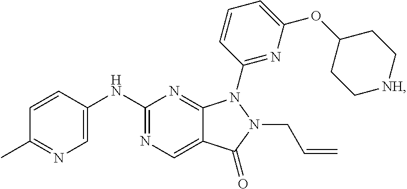 [SMILES: C=CCn1c(=O)c2cnc(Nc3ccc(C)nc3)nc2n1-c1cccc(OC2CCNCC2)n1] |
| 238 | 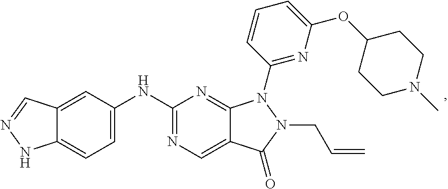 [SMILES: C=CCn1c(=O)c2cnc(Nc3ccc4[nH]ncc4c3)nc2n1-c1cccc(OC2CCN(C)CC2)n1] |
| 239 | 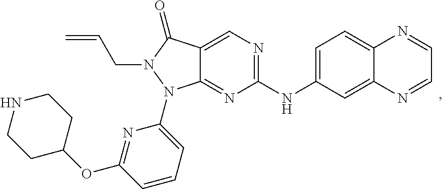 [SMILES: C=CCn1c(=O)c2cnc(Nc3ccc4nccnc4c3)nc2n1-c1cccc(OC2CCNCC2)n1] |
| 240 | 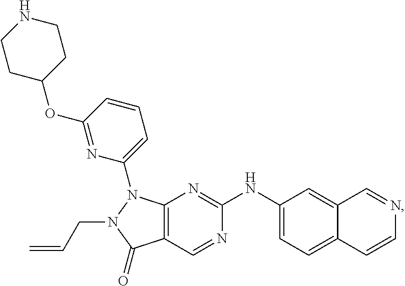 [SMILES: C=CCn1c(=O)c2cnc(Nc3ccc4ccncc4c3)nc2n1-c1cccc(OC2CCNCC2)n1] |
| 241 | 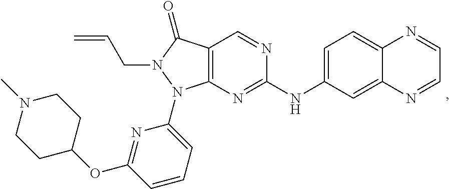 [SMILES: C=CCn1c(=O)c2cnc(Nc3ccc4nccnc4c3)nc2n1-c1cccc(OC2CCN(C)CC2)n1] |
| 242 | 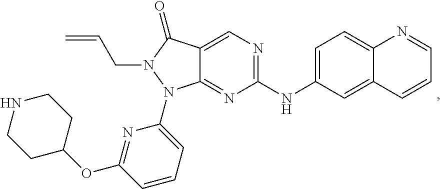 [SMILES: C=CCn1c(=O)c2cnc(Nc3ccc4ncccc4c3)nc2n1-c1cccc(OC2CCNCC2)n1] |
| 243 | 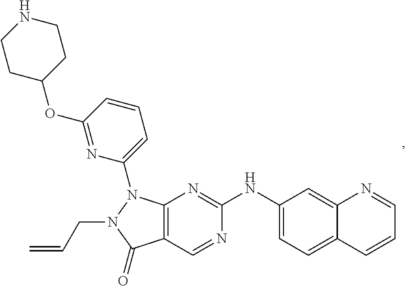 [SMILES: C=CCn1c(=O)c2cnc(Nc3ccc4cccnc4c3)nc2n1-c1cccc(OC2CCNCC2)n1] |
| 244 | 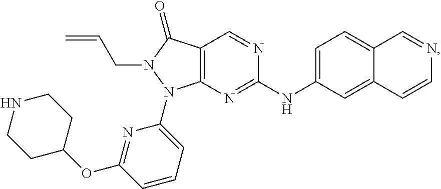 [SMILES: C=CCn1c(=O)c2cnc(Nc3ccc4cnccc4c3)nc2n1-c1cccc(OC2CCNCC2)n1] |
| 245 | 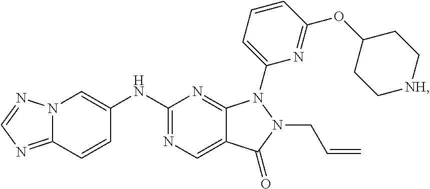 [SMILES: C=CCn1c(=O)c2cnc(Nc3ccc4ncnn4c3)nc2n1-c1cccc(OC2CCNCC2)n1] |
| 246 | 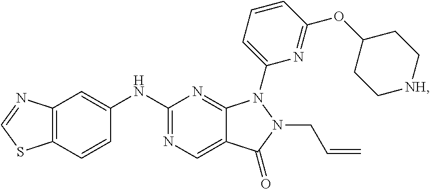 [SMILES: C=CCn1c(=O)c2cnc(Nc3ccc4scnc4c3)nc2n1-c1cccc(OC2CCNCC2)n1] |
| 247 | 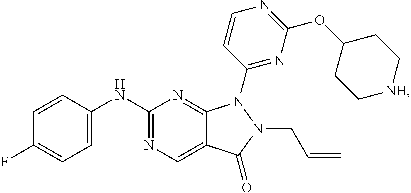 [SMILES: C=CCn1c(=O)c2cnc(Nc3ccc(F)cc3)nc2n1-c1ccnc(OC2CCNCC2)n1] |
| 248 | 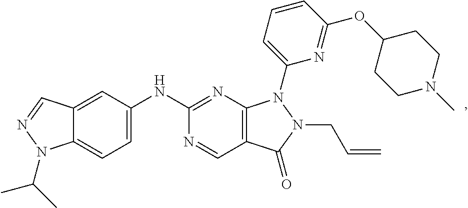 [SMILES: C=CCn1c(=O)c2cnc(Nc3ccc4c(cnn4C(C)C)c3)nc2n1-c1cccc(OC2CCN(C)CC2)n1] |
| 249 | 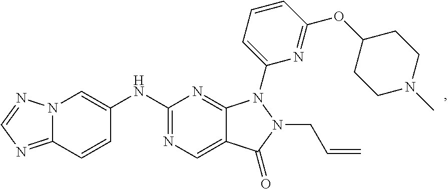 [SMILES: C=CCn1c(=O)c2cnc(Nc3ccc4ncnn4c3)nc2n1-c1cccc(OC2CCN(C)CC2)n1] |
| 250 | 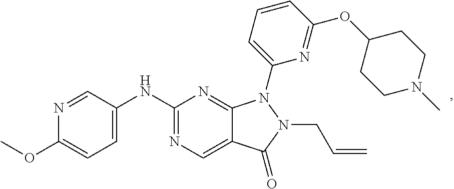 |
| 251 | 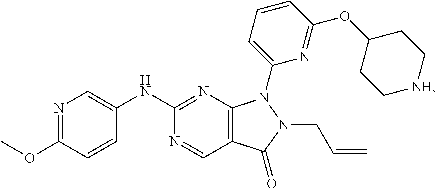 [SMILES: C=CCn1c(=O)c2cnc(Nc3ccc(OC)nc3)nc2n1-c1cccc(OC2CCNCC2)n1] |
| 252 | 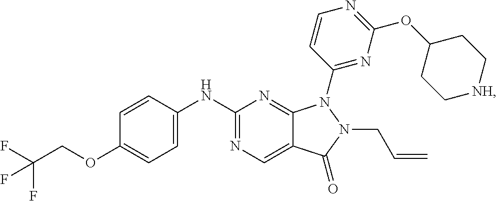 [SMILES: C=CCn1c(=O)c2cnc(Nc3ccc(OCC(F)(F)F)cc3)nc2n1-c1ccnc(OC2CCNCC2)n1] |
| 253 | 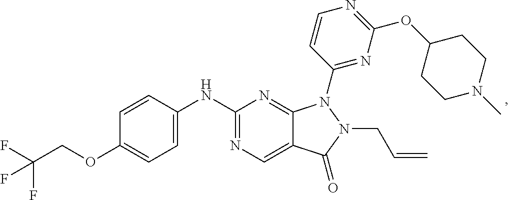 [SMILES: C=CCn1c(=O)c2cnc(Nc3ccc(OCC(F)(F)F)cc3)nc2n1-c1ccnc(OC2CCN(C)CC2)n1] |
| 254 | 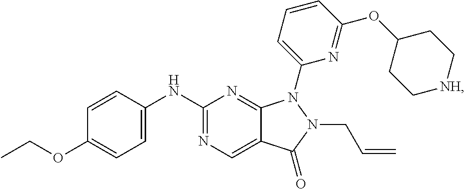 [SMILES: C=CCn1c(=O)c2cnc(Nc3ccc(OCC)cc3)nc2n1-c1cccc(OC2CCNCC2)n1] |
| 255 | 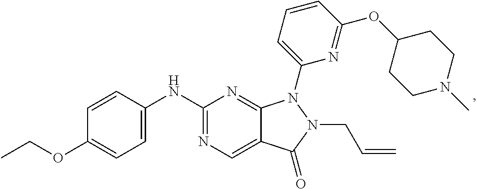 [SMILES: C=CCn1c(=O)c2cnc(Nc3ccc(OCC)cc3)nc2n1-c1cccc(OC2CCN(C)CC2)n1] |
| 256 | 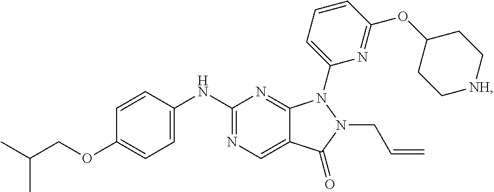 [SMILES: C=CCn1c(=O)c2cnc(Nc3ccc(OCC(C)C)cc3)nc2n1-c1cccc(OC2CCNCC2)n1] |
| 257 | 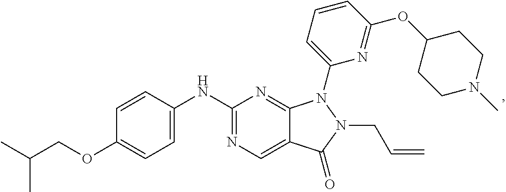 [SMILES: C=CCn1c(=O)c2cnc(Nc3ccc(OCC(C)C)cc3)nc2n1-c1cccc(OC2CCN(C)CC2)n1] |
| 258 | 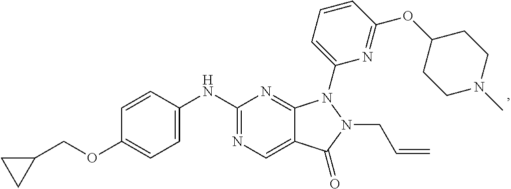 [SMILES: C=CCn1c(=O)c2cnc(Nc3ccc(OCC4CC4)cc3)nc2n1-c1cccc(OC2CCN(C)CC2)n1] |
| 259 | 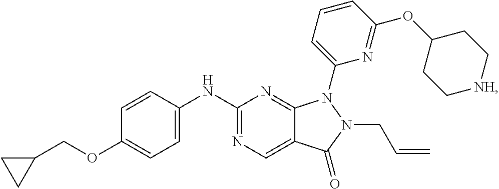 [SMILES: C=CCn1c(=O)c2cnc(Nc3ccc(OCC4CC4)cc3)nc2n1-c1cccc(OC2CCNCC2)n1] |
| 260 | 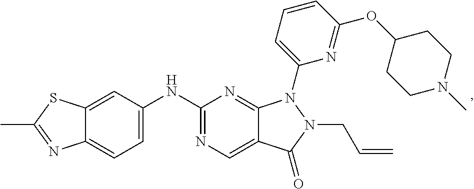 |
| 261 | 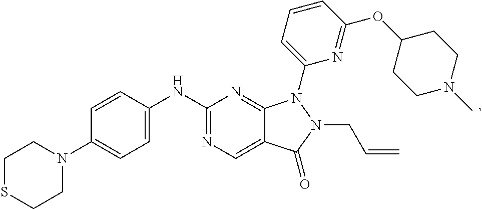 [SMILES: C=CCn1c(=O)c2cnc(Nc3ccc(N4CCSCC4)cc3)nc2n1-c1cccc(OC2CCN(C)CC2)n1] |
| 262 | 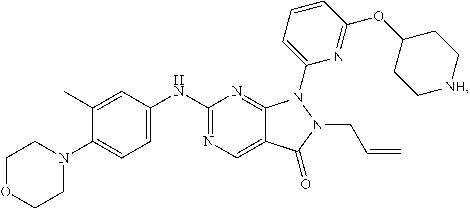 [SMILES: C=CCn1c(=O)c2cnc(Nc3ccc(N4CCOCC4)c(C)c3)nc2n1-c1cccc(OC2CCNCC2)n1] |
| 263 | 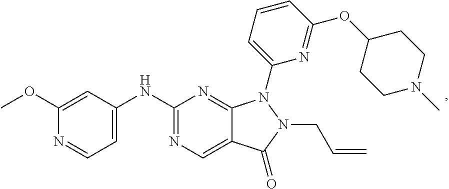 |
| 264 | 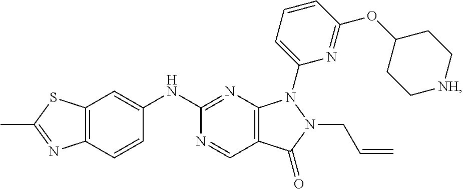 [SMILES: C=CCn1c(=O)c2cnc(Nc3ccc4nc(C)sc4c3)nc2n1-c1cccc(OC2CCNCC2)n1] |
| 265 | 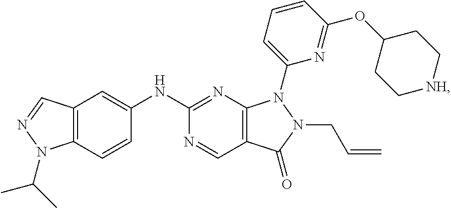 [SMILES: C=CCn1c(=O)c2cnc(Nc3ccc4c(cnn4C(C)C)c3)nc2n1-c1cccc(OC2CCNCC2)n1] |
| 266 | 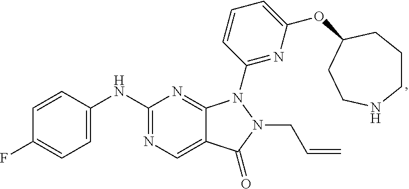 [SMILES: C=CCn1c(=O)c2cnc(Nc3ccc(F)cc3)nc2n1-c1cccc(O[C@H]2CCCNCC2)n1] |
| 267 | 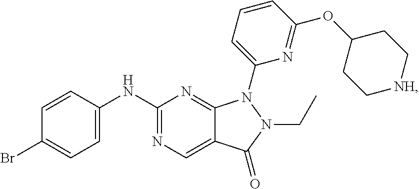 [SMILES: CCn1c(=O)c2cnc(Nc3ccc(Br)cc3)nc2n1-c1cccc(OC2CCNCC2)n1] |
| 268 | 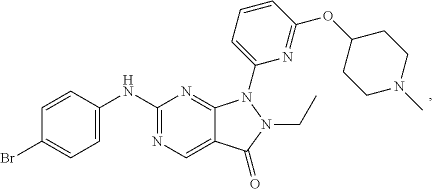 [SMILES: CCn1c(=O)c2cnc(Nc3ccc(Br)cc3)nc2n1-c1cccc(OC2CCN(C)CC2)n1] |
| 269 | 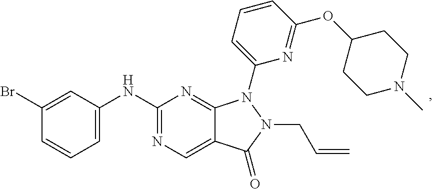 [SMILES: C=CCn1c(=O)c2cnc(Nc3cccc(Br)c3)nc2n1-c1cccc(OC2CCN(C)CC2)n1] |
| 270 | 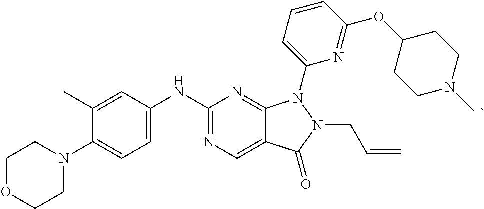 [SMILES: C=CCn1c(=O)c2cnc(Nc3ccc(N4CCOCC4)c(C)c3)nc2n1-c1cccc(OC2CCN(C)CC2)n1] |
| 271 | 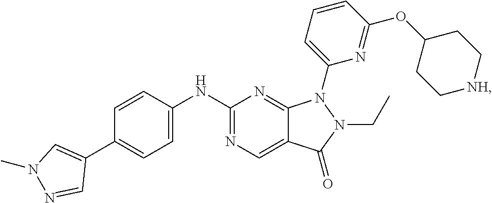 [SMILES: CCn1c(=O)c2cnc(Nc3ccc(-c4cnn(C)c4)cc3)nc2n1-c1cccc(OC2CCNCC2)n1] |
| 272 | 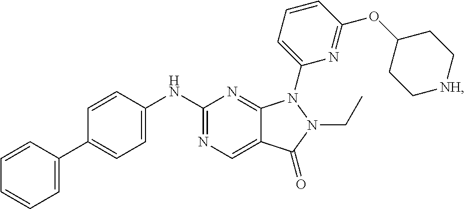 [SMILES: CCn1c(=O)c2cnc(Nc3ccc(-c4ccccc4)cc3)nc2n1-c1cccc(OC2CCNCC2)n1] |
| 273 | 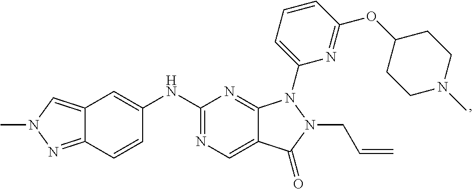 |
| 274 | 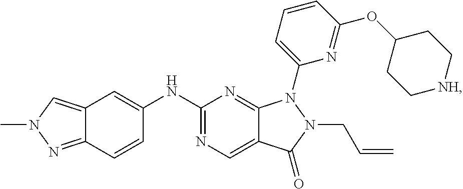 [SMILES: C=CCn1c(=O)c2cnc(Nc3ccc4nn(C)cc4c3)nc2n1-c1cccc(OC2CCNCC2)n1] |
| 275 | 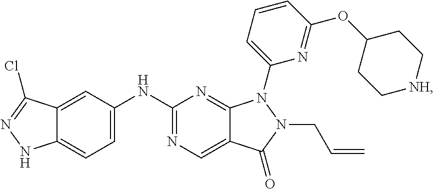 [SMILES: C=CCn1c(=O)c2cnc(Nc3ccc4[nH]nc(Cl)c4c3)nc2n1-c1cccc(OC2CCNCC2)n1] |
| 276 | 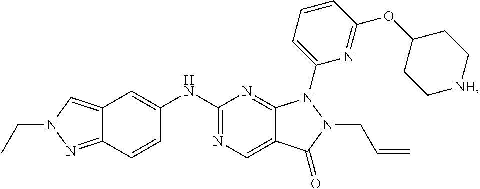 [SMILES: C=CCn1c(=O)c2cnc(Nc3ccc4nn(CC)cc4c3)nc2n1-c1cccc(OC2CCNCC2)n1] |
| 277 | 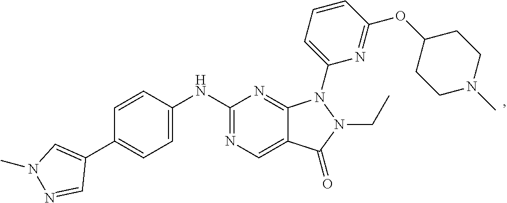 |
| 278 | 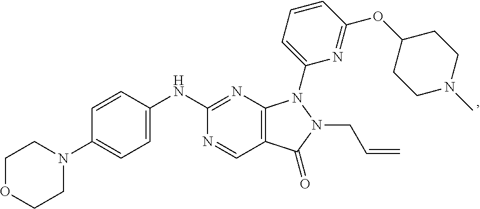 [SMILES: C=CCn1c(=O)c2cnc(Nc3ccc(N4CCOCC4)cc3)nc2n1-c1cccc(OC2CCN(C)CC2)n1] |
| 279 | 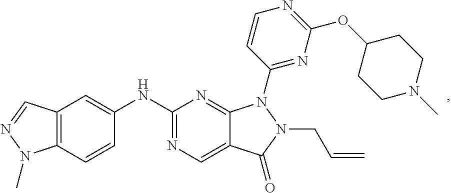 [SMILES: C=CCn1c(=O)c2cnc(Nc3ccc4c(cnn4C)c3)nc2n1-c1ccnc(OC2CCN(C)CC2)n1] |
| 280 | 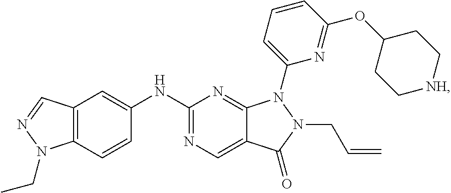 [SMILES: C=CCn1c(=O)c2cnc(Nc3ccc4c(cnn4CC)c3)nc2n1-c1cccc(OC2CCNCC2)n1] |
| 281 | 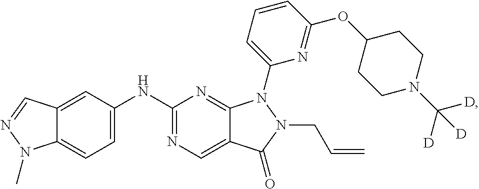 |
| 282 | 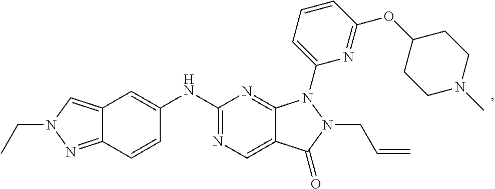 [SMILES: C=CCn1c(=O)c2cnc(Nc3ccc4nn(CC)cc4c3)nc2n1-c1cccc(OC2CCN(C)CC2)n1] |
| 284 | 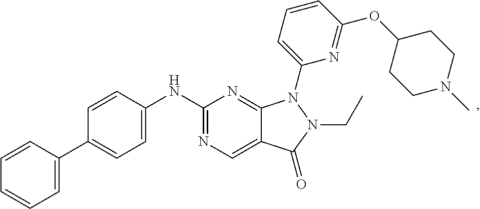 [SMILES: CCn1c(=O)c2cnc(Nc3ccc(-c4ccccc4)cc3)nc2n1-c1cccc(OC2CCN(C)CC2)n1] |
| 285 | 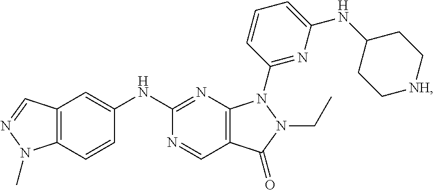 [SMILES: CCn1c(=O)c2cnc(Nc3ccc4c(cnn4C)c3)nc2n1-c1cccc(NC2CCNCC2)n1] |
| 286 | 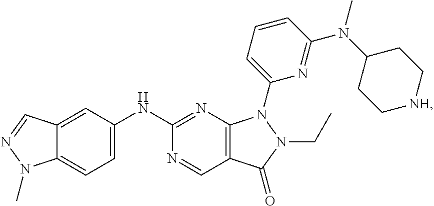 [SMILES: CCn1c(=O)c2cnc(Nc3ccc4c(cnn4C)c3)nc2n1-c1cccc(N(C)C2CCNCC2)n1] |
| 287 | 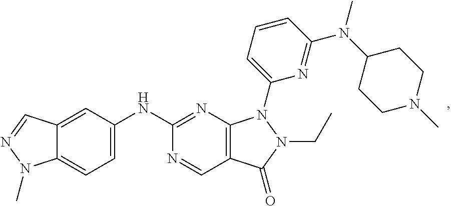 [SMILES: CCn1c(=O)c2cnc(Nc3ccc4c(cnn4C)c3)nc2n1-c1cccc(N(C)C2CCN(C)CC2)n1] |
| 288 | 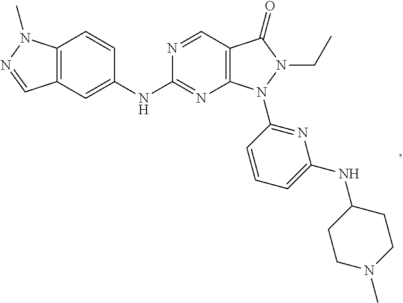 [SMILES: CCn1c(=O)c2cnc(Nc3ccc4c(cnn4C)c3)nc2n1-c1cccc(NC2CCN(C)CC2)n1] |
| 289 | 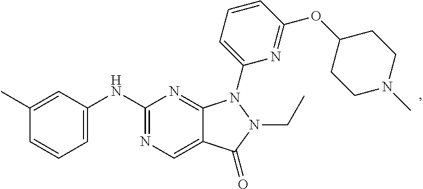 |
| 290 | 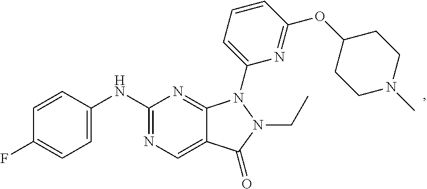 [SMILES: CCn1c(=O)c2cnc(Nc3ccc(F)cc3)nc2n1-c1cccc(OC2CCN(C)CC2)n1] |
| 291 | 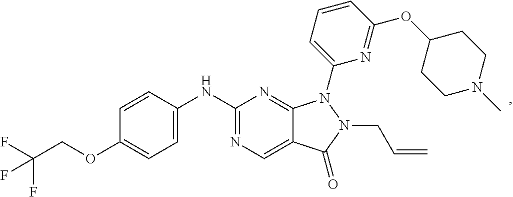 [SMILES: C=CCn1c(=O)c2cnc(Nc3ccc(OCC(F)(F)F)cc3)nc2n1-c1cccc(OC2CCN(C)CC2)n1] |
| 292 | 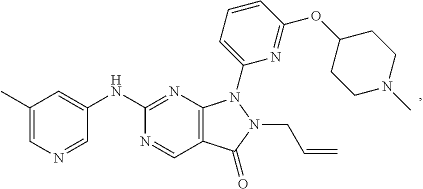 |
| 293 | 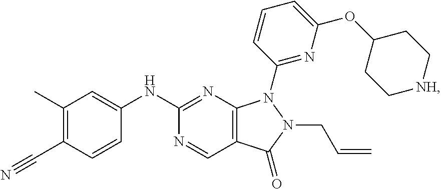 [SMILES: C=CCn1c(=O)c2cnc(Nc3ccc(C#N)c(C)c3)nc2n1-c1cccc(OC2CCNCC2)n1] |
| 294 | 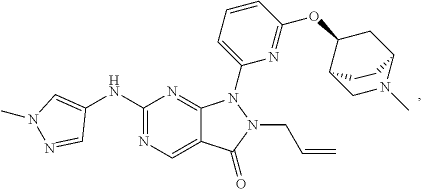 |
| 295 | 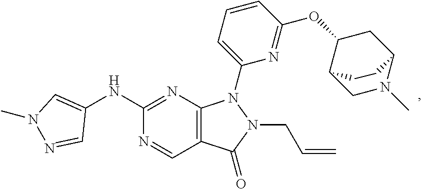 |
| 296 | 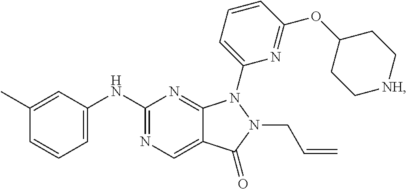 [SMILES: C=CCn1c(=O)c2cnc(Nc3cccc(C)c3)nc2n1-c1cccc(OC2CCNCC2)n1] |
| 297 | 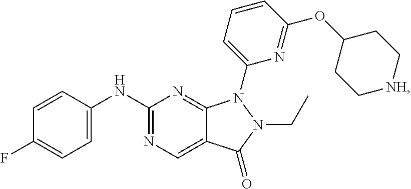 [SMILES: CCn1c(=O)c2cnc(Nc3ccc(F)cc3)nc2n1-c1cccc(OC2CCNCC2)n1] |
| 298 | 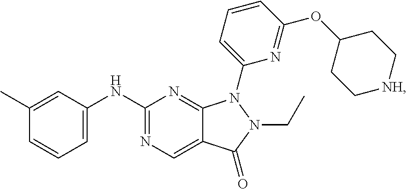 [SMILES: CCn1c(=O)c2cnc(Nc3cccc(C)c3)nc2n1-c1cccc(OC2CCNCC2)n1] |
| 299 | 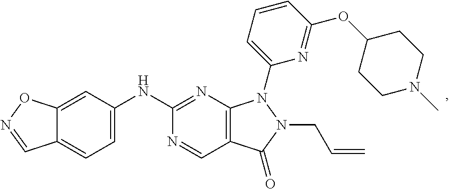 [SMILES: C=CCn1c(=O)c2cnc(Nc3ccc4cnoc4c3)nc2n1-c1cccc(OC2CCN(C)CC2)n1] |
| 300 | 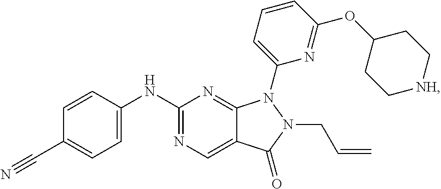 [SMILES: C=CCn1c(=O)c2cnc(Nc3ccc(C#N)cc3)nc2n1-c1cccc(OC2CCNCC2)n1] |
| 301 | 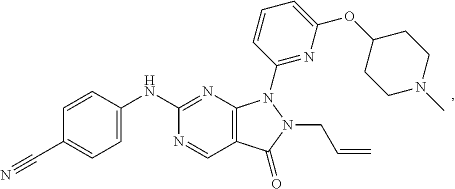 [SMILES: C=CCn1c(=O)c2cnc(Nc3ccc(C#N)cc3)nc2n1-c1cccc(OC2CCN(C)CC2)n1] |
| 302 | 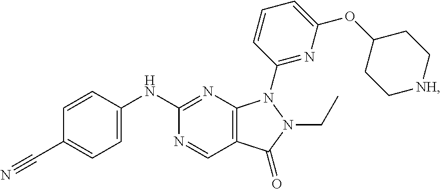 [SMILES: CCn1c(=O)c2cnc(Nc3ccc(C#N)cc3)nc2n1-c1cccc(OC2CCNCC2)n1] |
| 303 | 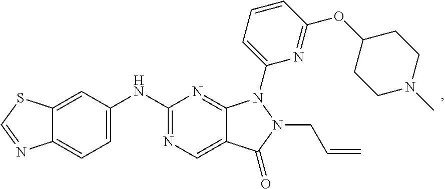 [SMILES: C=CCn1c(=O)c2cnc(Nc3ccc4ncsc4c3)nc2n1-c1cccc(OC2CCN(C)CC2)n1] |
| 304 | 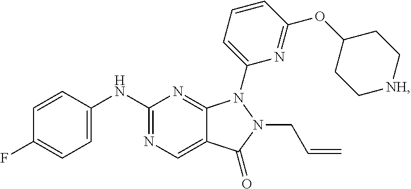 [SMILES: C=CCn1c(=O)c2cnc(Nc3ccc(F)cc3)nc2n1-c1cccc(OC2CCNCC2)n1] |
| 305 | 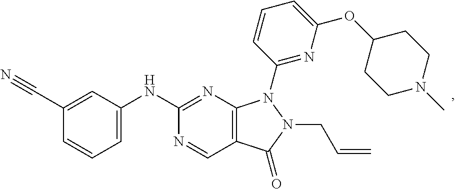 [SMILES: C=CCn1c(=O)c2cnc(Nc3cccc(C#N)c3)nc2n1-c1cccc(OC2CCN(C)CC2)n1] |
| 306 | 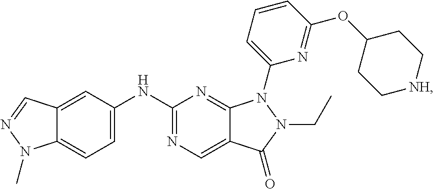 [SMILES: CCn1c(=O)c2cnc(Nc3ccc4c(cnn4C)c3)nc2n1-c1cccc(OC2CCNCC2)n1] |
| 307 | 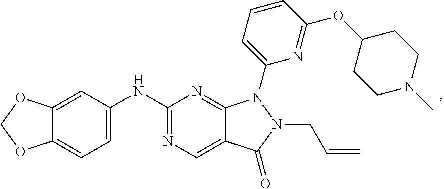 [SMILES: C=CCn1c(=O)c2cnc(Nc3ccc4c(c3)OCO4)nc2n1-c1cccc(OC2CCN(C)CC2)n1] |
| 308 | 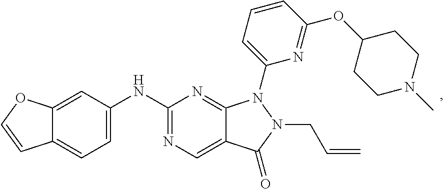 [SMILES: C=CCn1c(=O)c2cnc(Nc3ccc4ccoc4c3)nc2n1-c1cccc(OC2CCN(C)CC2)n1] |
| 309 | 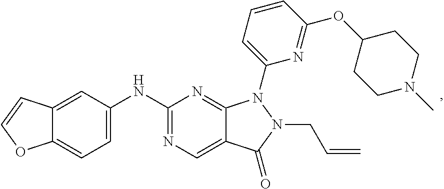 [SMILES: C=CCn1c(=O)c2cnc(Nc3ccc4occc4c3)nc2n1-c1cccc(OC2CCN(C)CC2)n1] |
| 310 | 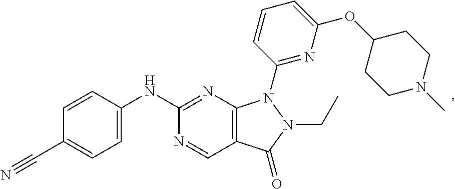 [SMILES: CCn1c(=O)c2cnc(Nc3ccc(C#N)cc3)nc2n1-c1cccc(OC2CCN(C)CC2)n1] |
| 311 | 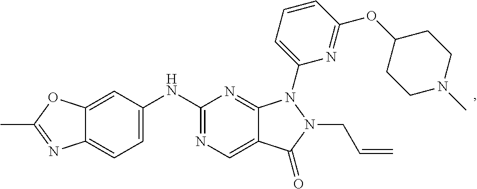 |
| 312 | 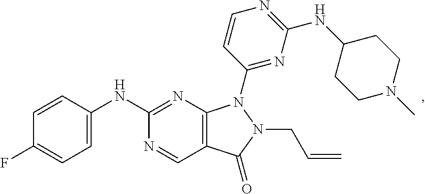 [SMILES: C=CCn1c(=O)c2cnc(Nc3ccc(F)cc3)nc2n1-c1ccnc(NC2CCN(C)CC2)n1] |
| 313 | 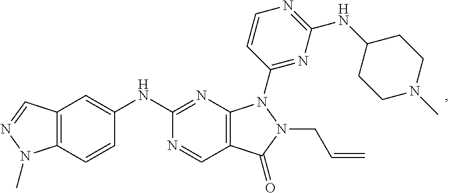 [SMILES: C=CCn1c(=O)c2cnc(Nc3ccc4c(cnn4C)c3)nc2n1-c1ccnc(NC2CCN(C)CC2)n1] |
| 314 | 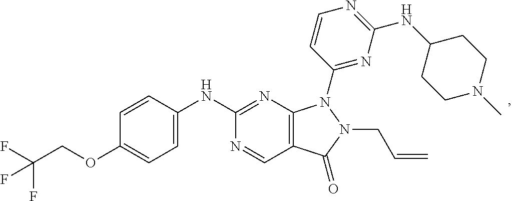 [SMILES: C=CCn1c(=O)c2cnc(Nc3ccc(OCC(F)(F)F)cc3)nc2n1-c1ccnc(NC2CCN(C)CC2)n1] |
| 315 | 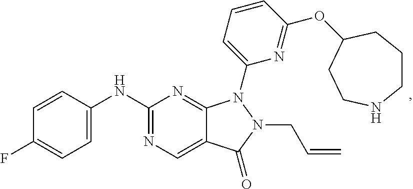 [SMILES: C=CCn1c(=O)c2cnc(Nc3ccc(F)cc3)nc2n1-c1cccc(OC2CCCNCC2)n1] |
| 316 | 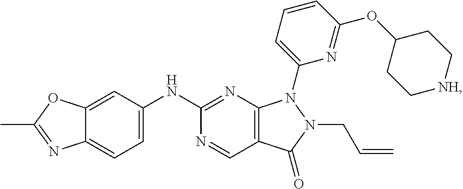 [SMILES: C=CCn1c(=O)c2cnc(Nc3ccc4nc(C)oc4c3)nc2n1-c1cccc(OC2CCNCC2)n1] |
| 317 | 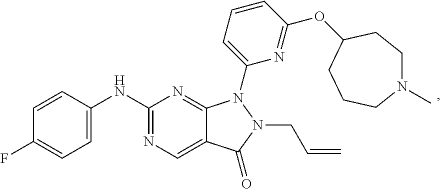 [SMILES: C=CCn1c(=O)c2cnc(Nc3ccc(F)cc3)nc2n1-c1cccc(OC2CCCN(C)CC2)n1] |
| 318 | 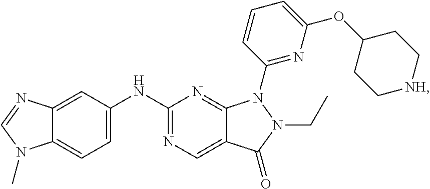 [SMILES: CCn1c(=O)c2cnc(Nc3ccc4c(c3)ncn4C)nc2n1-c1cccc(OC2CCNCC2)n1] |
| 319 | 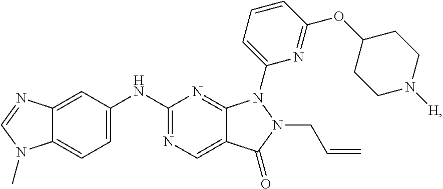 |
| 320 | 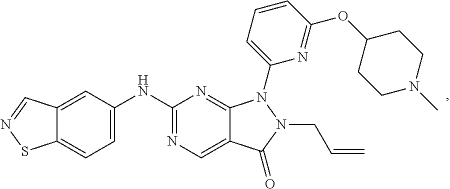 [SMILES: C=CCn1c(=O)c2cnc(Nc3ccc4sncc4c3)nc2n1-c1cccc(OC2CCN(C)CC2)n1] |
| 321 | 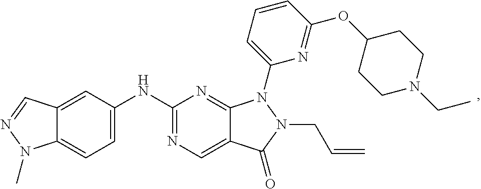 [SMILES: C=CCn1c(=O)c2cnc(Nc3ccc4c(cnn4C)c3)nc2n1-c1cccc(OC2CCN(CC)CC2)n1] |
| 322 | 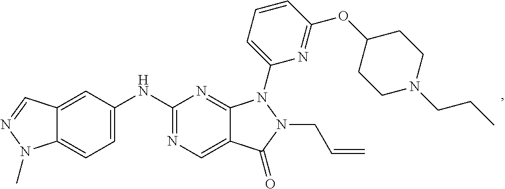 [SMILES: C=CCn1c(=O)c2cnc(Nc3ccc4c(cnn4C)c3)nc2n1-c1cccc(OC2CCN(CCC)CC2)n1] |
| 323 | 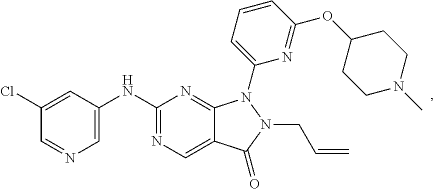 [SMILES: C=CCn1c(=O)c2cnc(Nc3cncc(Cl)c3)nc2n1-c1cccc(OC2CCN(C)CC2)n1] |
| 324 | 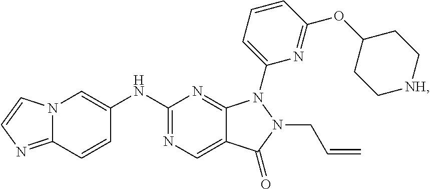 [SMILES: C=CCn1c(=O)c2cnc(Nc3ccc4nccn4c3)nc2n1-c1cccc(OC2CCNCC2)n1] |
| 325 | 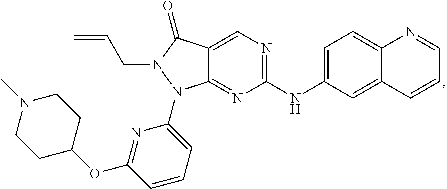 [SMILES: C=CCn1c(=O)c2cnc(Nc3ccc4ncccc4c3)nc2n1-c1cccc(OC2CCN(C)CC2)n1] |
| 326 | 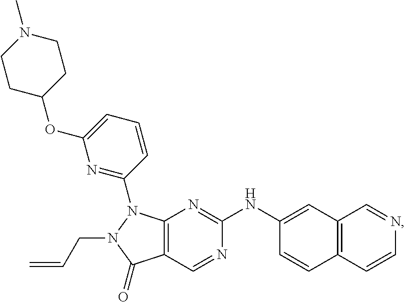 [SMILES: C=CCn1c(=O)c2cnc(Nc3ccc4ccncc4c3)nc2n1-c1cccc(OC2CCN(C)CC2)n1] |
| 327 | 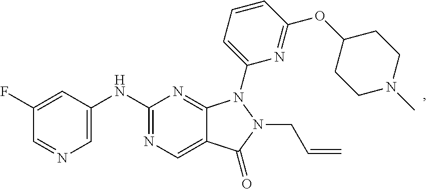 [SMILES: C=CCn1c(=O)c2cnc(Nc3cncc(F)c3)nc2n1-c1cccc(OC2CCN(C)CC2)n1] |
| 328 | 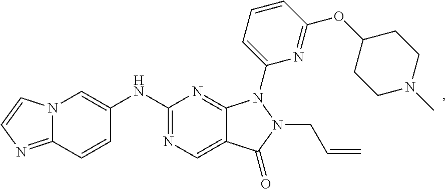 [SMILES: C=CCn1c(=O)c2cnc(Nc3ccc4nccn4c3)nc2n1-c1cccc(OC2CCN(C)CC2)n1] |
| 329 | 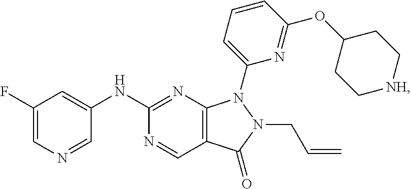 [SMILES: C=CCn1c(=O)c2cnc(Nc3cncc(F)c3)nc2n1-c1cccc(OC2CCNCC2)n1] |
| 330 | 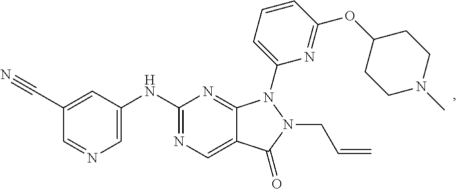 [SMILES: C=CCn1c(=O)c2cnc(Nc3cncc(C#N)c3)nc2n1-c1cccc(OC2CCN(C)CC2)n1] |
| 331 | 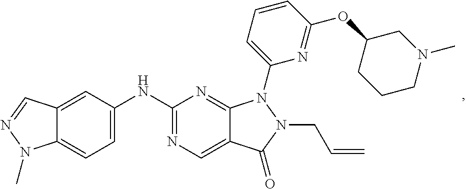 [SMILES: C=CCn1c(=O)c2cnc(Nc3ccc4c(cnn4C)c3)nc2n1-c1cccc(O[C@@H]2CCCN(C)C2)n1] |
| 332 | 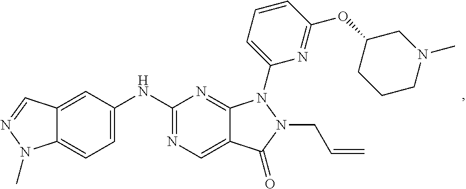 [SMILES: C=CCn1c(=O)c2cnc(Nc3ccc4c(cnn4C)c3)nc2n1-c1cccc(O[C@H]2CCCN(C)C2)n1] |
| 333 | 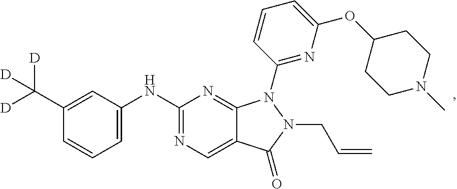 [SMILES: [2H]C([2H])([2H])c1cccc(Nc2ncc3c(=O)n(CC=C)n(-c4cccc(OC5CCN(C)CC5)n4)c3n2)c1] |
| 334 | 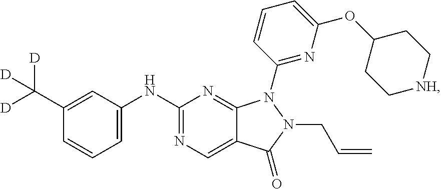 [SMILES: [2H]C([2H])([2H])c1cccc(Nc2ncc3c(=O)n(CC=C)n(-c4cccc(OC5CCNCC5)n4)c3n2)c1] |
| 335 | 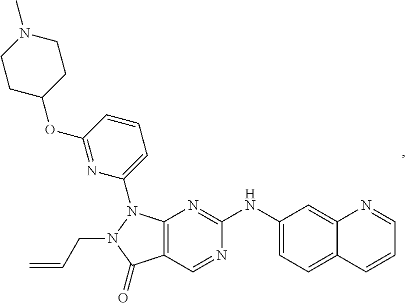 [SMILES: C=CCn1c(=O)c2cnc(Nc3ccc4cccnc4c3)nc2n1-c1cccc(OC2CCN(C)CC2)n1] |
| 336 | 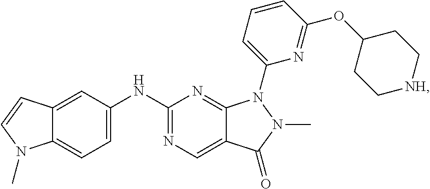 [SMILES: Cn1ccc2cc(Nc3ncc4c(=O)n(C)n(-c5cccc(OC6CCNCC6)n5)c4n3)ccc21] |
| 337 | 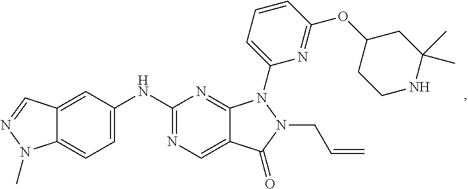 [SMILES: C=CCn1c(=O)c2cnc(Nc3ccc4c(cnn4C)c3)nc2n1-c1cccc(OC2CCNC(C)(C)C2)n1] |
| 338 | 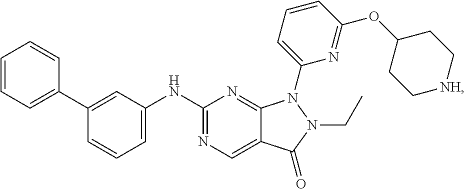 [SMILES: CCn1c(=O)c2cnc(Nc3cccc(-c4ccccc4)c3)nc2n1-c1cccc(OC2CCNCC2)n1] |
| 339 | 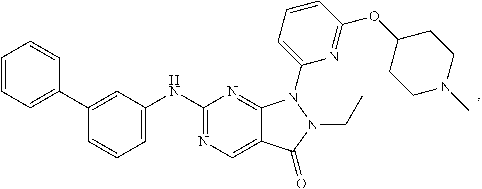 [SMILES: CCn1c(=O)c2cnc(Nc3cccc(-c4ccccc4)c3)nc2n1-c1cccc(OC2CCN(C)CC2)n1] |
| 340 | 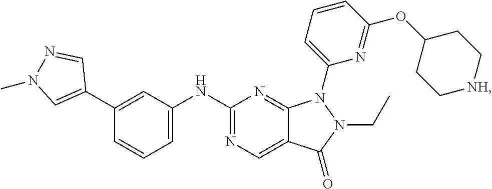 [SMILES: CCn1c(=O)c2cnc(Nc3cccc(-c4cnn(C)c4)c3)nc2n1-c1cccc(OC2CCNCC2)n1] |
| 341 | 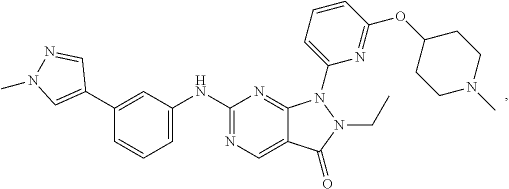 |
| 342 | 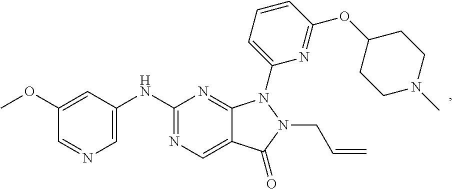 |
| 343 | 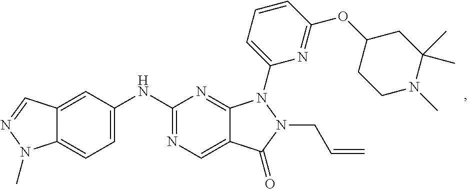 [SMILES: C=CCn1c(=O)c2cnc(Nc3ccc4c(cnn4C)c3)nc2n1-c1cccc(OC2CCN(C)C(C)(C)C2)n1] |
| 344 | 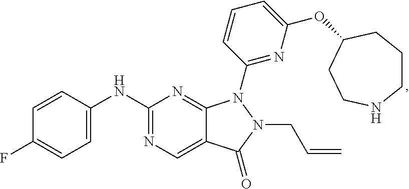 [SMILES: C=CCn1c(=O)c2cnc(Nc3ccc(F)cc3)nc2n1-c1cccc(O[C@@H]2CCCNCC2)n1] |
| 345 | 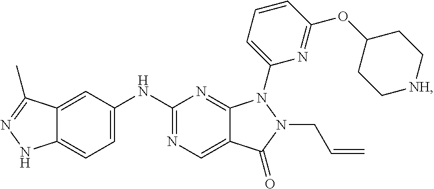 [SMILES: C=CCn1c(=O)c2cnc(Nc3ccc4[nH]nc(C)c4c3)nc2n1-c1cccc(OC2CCNCC2)n1] |
| 346 | 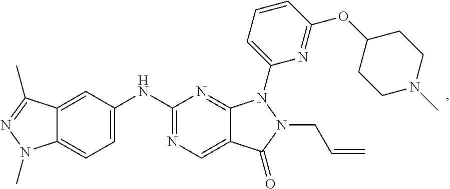 [SMILES: C=CCn1c(=O)c2cnc(Nc3ccc4c(c3)c(C)nn4C)nc2n1-c1cccc(OC2CCN(C)CC2)n1] |
| 347 | 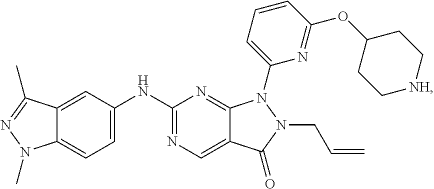 [SMILES: C=CCn1c(=O)c2cnc(Nc3ccc4c(c3)c(C)nn4C)nc2n1-c1cccc(OC2CCNCC2)n1] |
| 348 | 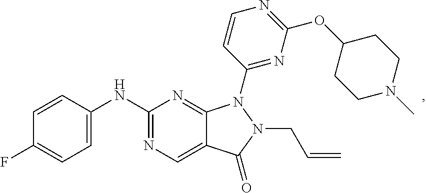 [SMILES: C=CCn1c(=O)c2cnc(Nc3ccc(F)cc3)nc2n1-c1ccnc(OC2CCN(C)CC2)n1] |
| 349 | 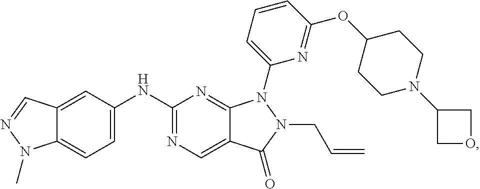 [SMILES: C=CCn1c(=O)c2cnc(Nc3ccc4c(cnn4C)c3)nc2n1-c1cccc(OC2CCN(C3COC3)CC2)n1] |
| 350 | 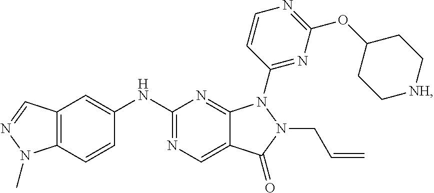 [SMILES: C=CCn1c(=O)c2cnc(Nc3ccc4c(cnn4C)c3)nc2n1-c1ccnc(OC2CCNCC2)n1] |
| 351 | 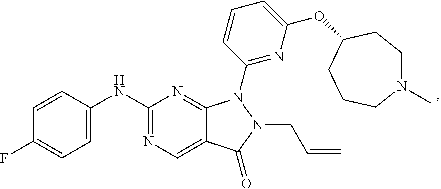 [SMILES: C=CCn1c(=O)c2cnc(Nc3ccc(F)cc3)nc2n1-c1cccc(O[C@H]2CCCN(C)CC2)n1] |
| 352 | 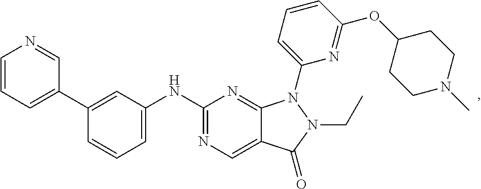 [SMILES: CCn1c(=O)c2cnc(Nc3cccc(-c4cccnc4)c3)nc2n1-c1cccc(OC2CCN(C)CC2)n1] |
| 353 | 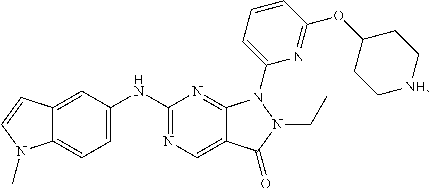 [SMILES: CCn1c(=O)c2cnc(Nc3ccc4c(ccn4C)c3)nc2n1-c1cccc(OC2CCNCC2)n1] |
| 354 | 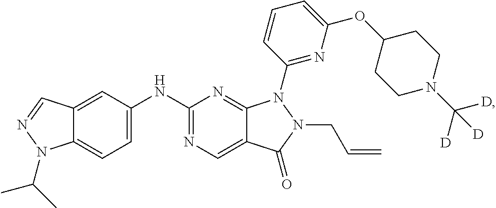 |
| 355 | 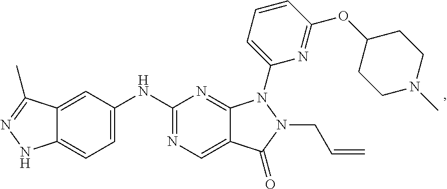 [SMILES: C=CCn1c(=O)c2cnc(Nc3ccc4[nH]nc(C)c4c3)nc2n1-c1cccc(OC2CCN(C)CC2)n1] |
| 356 | 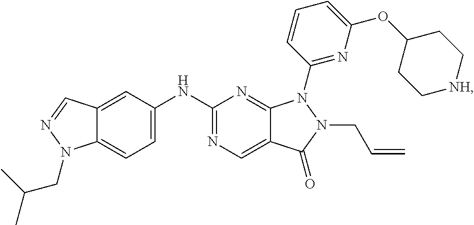 [SMILES: C=CCn1c(=O)c2cnc(Nc3ccc4c(cnn4CC(C)C)c3)nc2n1-c1cccc(OC2CCNCC2)n1] |
| 357 | 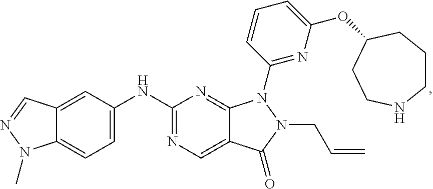 [SMILES: C=CCn1c(=O)c2cnc(Nc3ccc4c(cnn4C)c3)nc2n1-c1cccc(O[C@@H]2CCCNCC2)n1] |
| 358 | 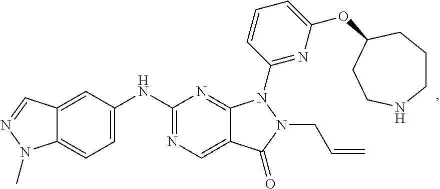 [SMILES: C=CCn1c(=O)c2cnc(Nc3ccc4c(cnn4C)c3)nc2n1-c1cccc(O[C@H]2CCCNCC2)n1] |
| 359 | 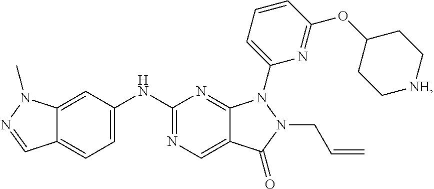 [SMILES: C=CCn1c(=O)c2cnc(Nc3ccc4cnn(C)c4c3)nc2n1-c1cccc(OC2CCNCC2)n1] |
| 360 | 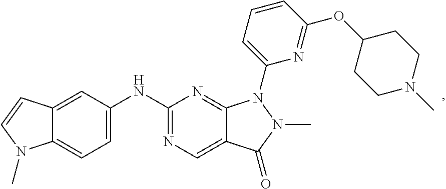 [SMILES: CN1CCC(Oc2cccc(-n3c4nc(Nc5ccc6c(ccn6C)c5)ncc4c(=O)n3C)n2)CC1] |
| 361 | 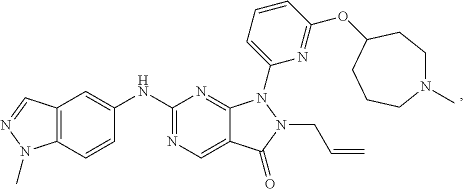 [SMILES: C=CCn1c(=O)c2cnc(Nc3ccc4c(cnn4C)c3)nc2n1-c1cccc(OC2CCCN(C)CC2)n1] |
| 362 | 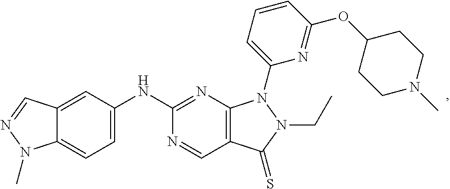 [SMILES: CCn1c(=S)c2cnc(Nc3ccc4c(cnn4C)c3)nc2n1-c1cccc(OC2CCN(C)CC2)n1] |
| 363 | 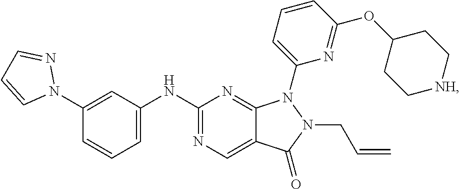 [SMILES: C=CCn1c(=O)c2cnc(Nc3cccc(-n4cccn4)c3)nc2n1-c1cccc(OC2CCNCC2)n1] |
| 364 | 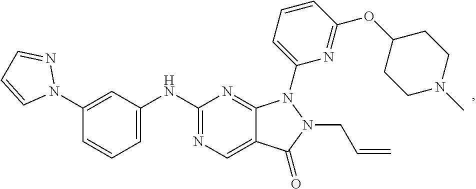 [SMILES: C=CCn1c(=O)c2cnc(Nc3cccc(-n4cccn4)c3)nc2n1-c1cccc(OC2CCN(C)CC2)n1] |
| 365 | 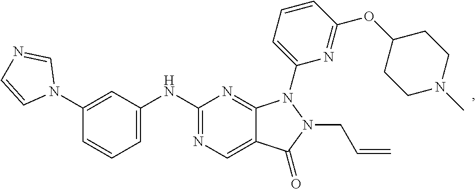 [SMILES: C=CCn1c(=O)c2cnc(Nc3cccc(-n4ccnc4)c3)nc2n1-c1cccc(OC2CCN(C)CC2)n1] |
| 366 | 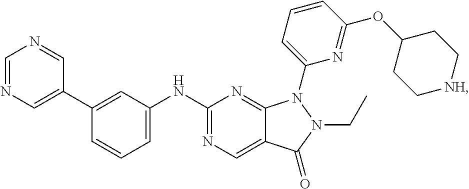 [SMILES: CCn1c(=O)c2cnc(Nc3cccc(-c4cncnc4)c3)nc2n1-c1cccc(OC2CCNCC2)n1] |
| 367 | 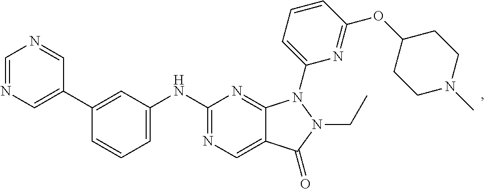 [SMILES: CCn1c(=O)c2cnc(Nc3cccc(-c4cncnc4)c3)nc2n1-c1cccc(OC2CCN(C)CC2)n1] |
| 368 | 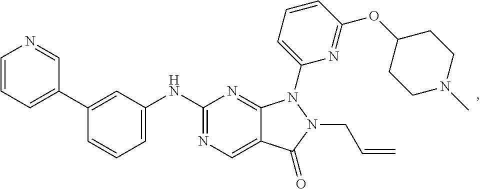 [SMILES: C=CCn1c(=O)c2cnc(Nc3cccc(-c4cccnc4)c3)nc2n1-c1cccc(OC2CCN(C)CC2)n1] |
| 369 | 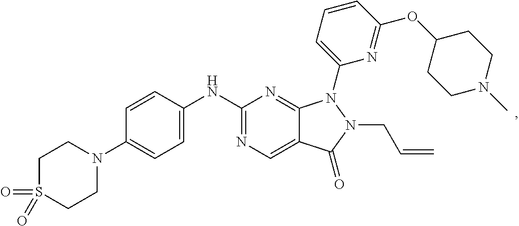 [SMILES: C=CCn1c(=O)c2cnc(Nc3ccc(N4CCS(=O)(=O)CC4)cc3)nc2n1-c1cccc(OC2CCN(C)CC2)n1] |
| 370 | 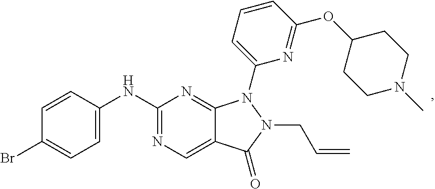 [SMILES: C=CCn1c(=O)c2cnc(Nc3ccc(Br)cc3)nc2n1-c1cccc(OC2CCN(C)CC2)n1] |
| 371 | 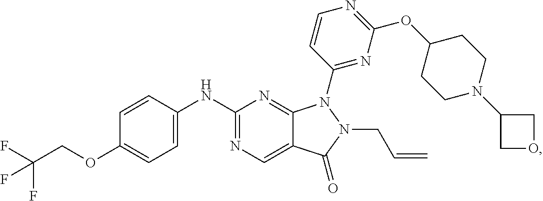 [SMILES: C=CCn1c(=O)c2cnc(Nc3ccc(OCC(F)(F)F)cc3)nc2n1-c1ccnc(OC2CCN(C3COC3)CC2)n1] |
| 372 | 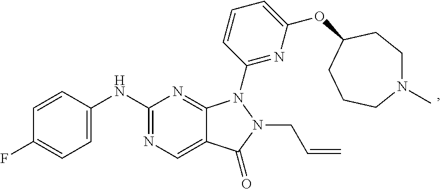 [SMILES: C=CCn1c(=O)c2cnc(Nc3ccc(F)cc3)nc2n1-c1cccc(O[C@@H]2CCCN(C)CC2)n1] |
| 373 | 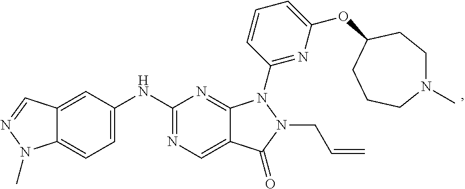 [SMILES: C=CCn1c(=O)c2cnc(Nc3ccc4c(cnn4C)c3)nc2n1-c1cccc(O[C@@H]2CCCN(C)CC2)n1] |
| 374 | 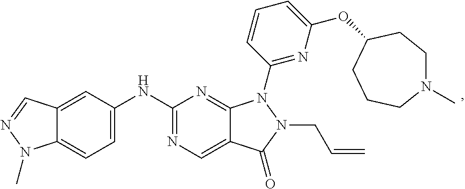 [SMILES: C=CCn1c(=O)c2cnc(Nc3ccc4c(cnn4C)c3)nc2n1-c1cccc(O[C@H]2CCCN(C)CC2)n1] |
| 375 | 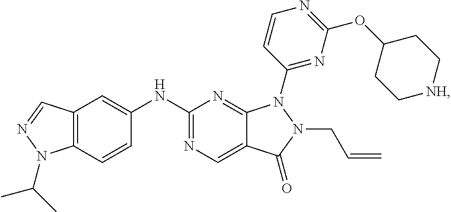 [SMILES: C=CCn1c(=O)c2cnc(Nc3ccc4c(cnn4C(C)C)c3)nc2n1-c1ccnc(OC2CCNCC2)n1] |
| 376 | 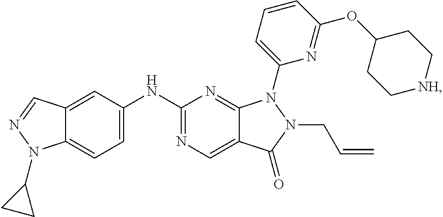 [SMILES: C=CCn1c(=O)c2cnc(Nc3ccc4c(cnn4C4CC4)c3)nc2n1-c1cccc(OC2CCNCC2)n1] |
| 377 | 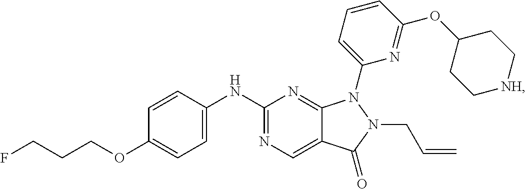 [SMILES: C=CCn1c(=O)c2cnc(Nc3ccc(OCCCF)cc3)nc2n1-c1cccc(OC2CCNCC2)n1] |
| 378 | 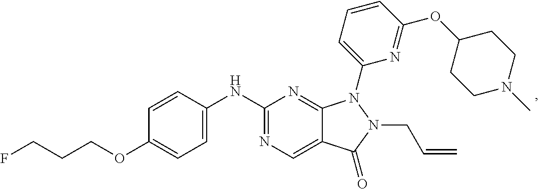 [SMILES: C=CCn1c(=O)c2cnc(Nc3ccc(OCCCF)cc3)nc2n1-c1cccc(OC2CCN(C)CC2)n1] |
| 379 | 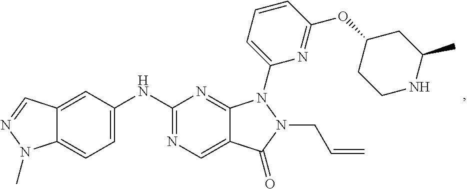 [SMILES: C=CCn1c(=O)c2cnc(Nc3ccc4c(cnn4C)c3)nc2n1-c1cccc(O[C@H]2CCN[C@H](C)C2)n1] |
| 380 | 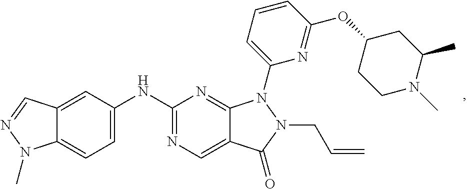 [SMILES: C=CCn1c(=O)c2cnc(Nc3ccc4c(cnn4C)c3)nc2n1-c1cccc(O[C@H]2CCN(C)[C@H](C)C2)n1] |
| 381 | 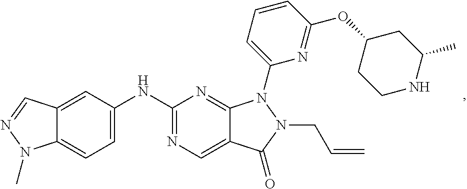 [SMILES: C=CCn1c(=O)c2cnc(Nc3ccc4c(cnn4C)c3)nc2n1-c1cccc(O[C@H]2CCN[C@@H](C)C2)n1] |
| 382 | 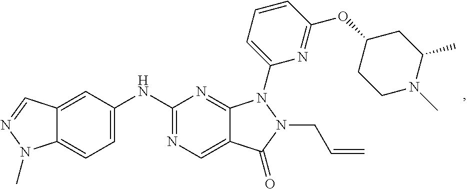 [SMILES: C=CCn1c(=O)c2cnc(Nc3ccc4c(cnn4C)c3)nc2n1-c1cccc(O[C@H]2CCN(C)[C@@H](C)C2)n1] |
| 383 | 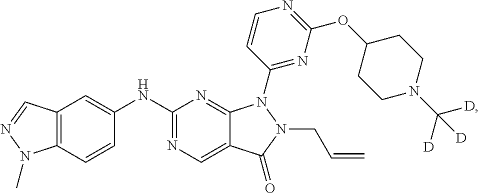 |
| 384 | 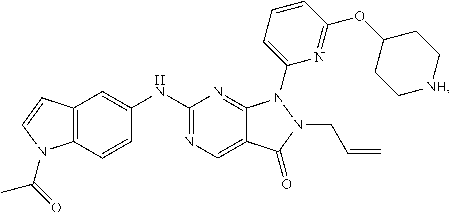 [SMILES: C=CCn1c(=O)c2cnc(Nc3ccc4c(ccn4C(C)=O)c3)nc2n1-c1cccc(OC2CCNCC2)n1] |
| 385 | 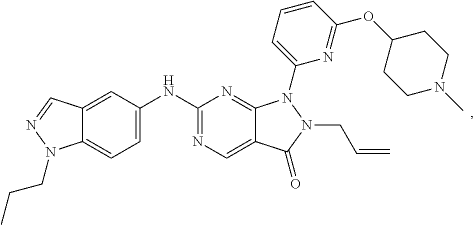 [SMILES: C=CCn1c(=O)c2cnc(Nc3ccc4c(cnn4CCC)c3)nc2n1-c1cccc(OC2CCN(C)CC2)n1] |
| 386 | 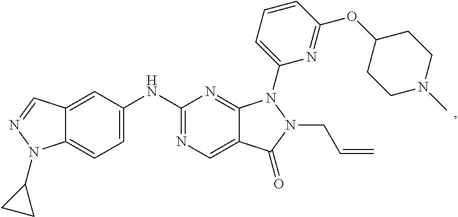 [SMILES: C=CCn1c(=O)c2cnc(Nc3ccc4c(cnn4C4CC4)c3)nc2n1-c1cccc(OC2CCN(C)CC2)n1] |
| 387 | 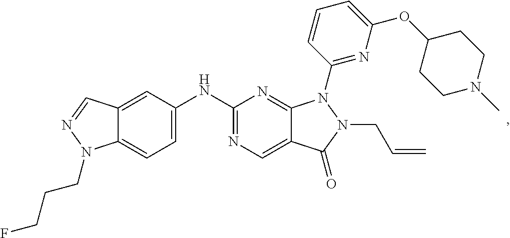 [SMILES: C=CCn1c(=O)c2cnc(Nc3ccc4c(cnn4CCCF)c3)nc2n1-c1cccc(OC2CCN(C)CC2)n1] |
| 388 | 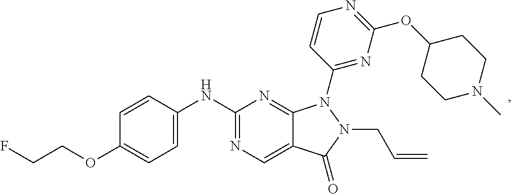 [SMILES: C=CCn1c(=O)c2cnc(Nc3ccc(OCCF)cc3)nc2n1-c1ccnc(OC2CCN(C)CC2)n1] |
| 389 | 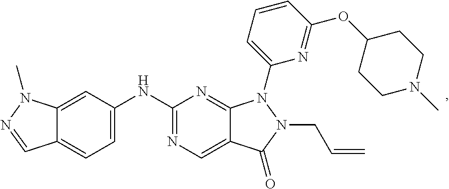 [SMILES: C=CCn1c(=O)c2cnc(Nc3ccc4cnn(C)c4c3)nc2n1-c1cccc(OC2CCN(C)CC2)n1] |
| 390 | 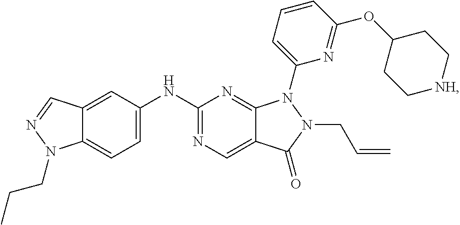 [SMILES: C=CCn1c(=O)c2cnc(Nc3ccc4c(cnn4CCC)c3)nc2n1-c1cccc(OC2CCNCC2)n1] |
| 391 | 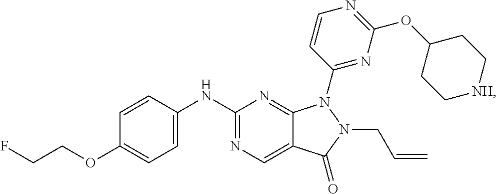 [SMILES: C=CCn1c(=O)c2cnc(Nc3ccc(OCCF)cc3)nc2n1-c1ccnc(OC2CCNCC2)n1] |
| 392 | 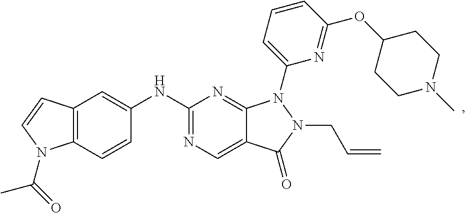 [SMILES: C=CCn1c(=O)c2cnc(Nc3ccc4c(ccn4C(C)=O)c3)nc2n1-c1cccc(OC2CCN(C)CC2)n1] |
| 393 | 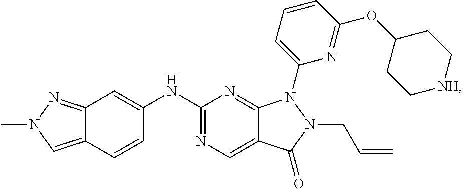 [SMILES: C=CCn1c(=O)c2cnc(Nc3ccc4cn(C)nc4c3)nc2n1-c1cccc(OC2CCNCC2)n1] |
| 394 | 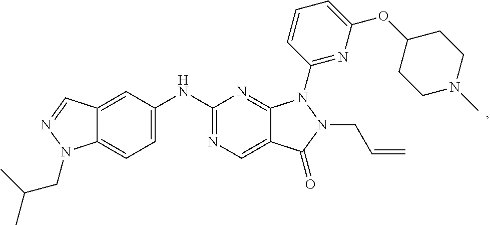 [SMILES: C=CCn1c(=O)c2cnc(Nc3ccc4c(cnn4CC(C)C)c3)nc2n1-c1cccc(OC2CCN(C)CC2)n1] |
| 395 | 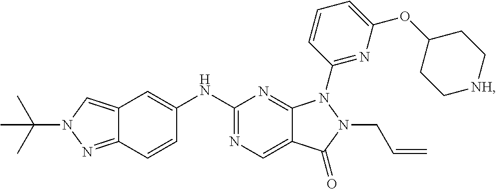 [SMILES: C=CCn1c(=O)c2cnc(Nc3ccc4nn(C(C)(C)C)cc4c3)nc2n1-c1cccc(OC2CCNCC2)n1] |
| 396 | 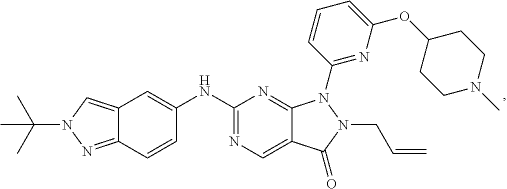 [SMILES: C=CCn1c(=O)c2cnc(Nc3ccc4nn(C(C)(C)C)cc4c3)nc2n1-c1cccc(OC2CCN(C)CC2)n1] |
| 397 | 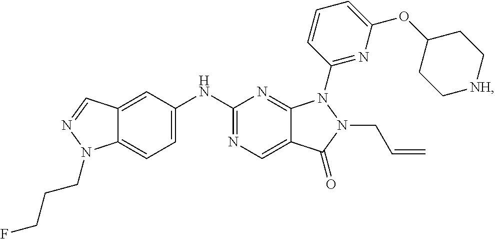 [SMILES: C=CCn1c(=O)c2cnc(Nc3ccc4c(cnn4CCCF)c3)nc2n1-c1cccc(OC2CCNCC2)n1] |
| 398 | 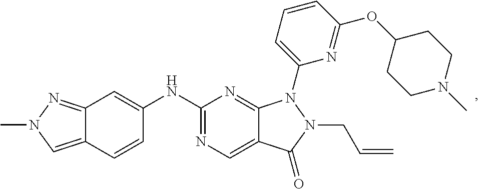 |
| 399 | 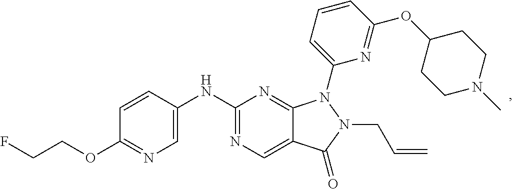 [SMILES: C=CCn1c(=O)c2cnc(Nc3ccc(OCCF)nc3)nc2n1-c1cccc(OC2CCN(C)CC2)n1] |
| 400 | 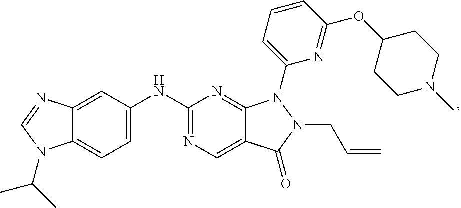 [SMILES: C=CCn1c(=O)c2cnc(Nc3ccc4c(c3)ncn4C(C)C)nc2n1-c1cccc(OC2CCN(C)CC2)n1] |
| 401 | 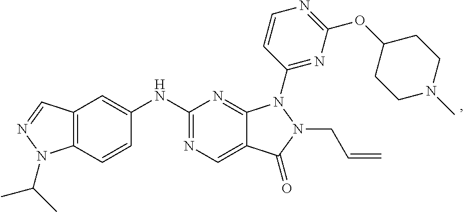 [SMILES: C=CCn1c(=O)c2cnc(Nc3ccc4c(cnn4C(C)C)c3)nc2n1-c1ccnc(OC2CCN(C)CC2)n1] |
| 402 | 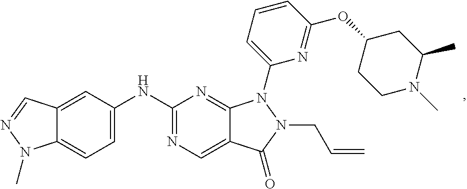 [SMILES: C=CCn1c(=O)c2cnc(Nc3ccc4c(cnn4C)c3)nc2n1-c1cccc(O[C@H]2CCN(C)[C@H](C)C2)n1] |
| 403 | 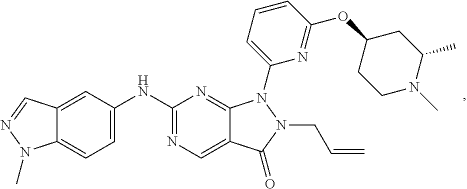 [SMILES: C=CCn1c(=O)c2cnc(Nc3ccc4c(cnn4C)c3)nc2n1-c1cccc(O[C@@H]2CCN(C)[C@@H](C)C2)n1] |
| 404 | 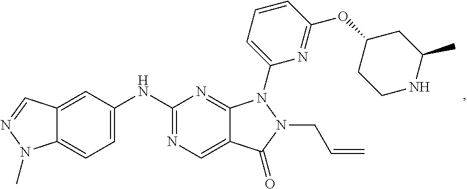 [SMILES: C=CCn1c(=O)c2cnc(Nc3ccc4c(cnn4C)c3)nc2n1-c1cccc(O[C@H]2CCN[C@H](C)C2)n1] |
| 405 | 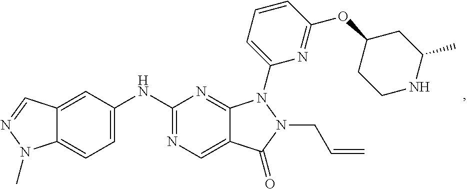 [SMILES: C=CCn1c(=O)c2cnc(Nc3ccc4c(cnn4C)c3)nc2n1-c1cccc(O[C@@H]2CCN[C@@H](C)C2)n1] |
| 406 | 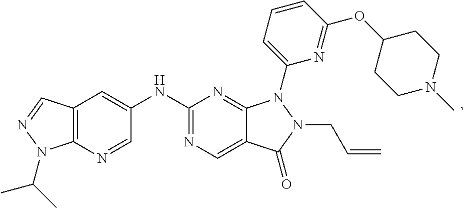 [SMILES: C=CCn1c(=O)c2cnc(Nc3cnc4c(cnn4C(C)C)c3)nc2n1-c1cccc(OC2CCN(C)CC2)n1] |
| 407 | 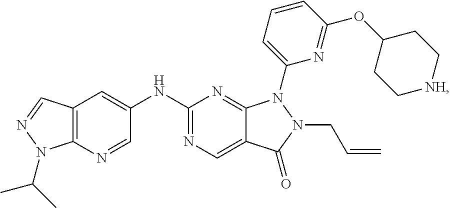 [SMILES: C=CCn1c(=O)c2cnc(Nc3cnc4c(cnn4C(C)C)c3)nc2n1-c1cccc(OC2CCNCC2)n1] |
| 408 | 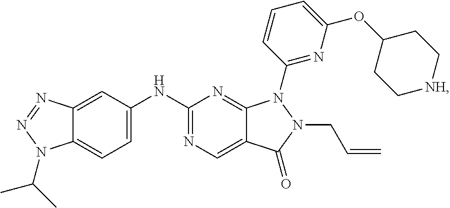 [SMILES: C=CCn1c(=O)c2cnc(Nc3ccc4c(c3)nnn4C(C)C)nc2n1-c1cccc(OC2CCNCC2)n1] |
| | or |
| 409 | 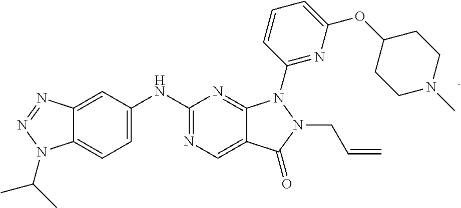 [SMILES: C=CCn1c(=O)c2cnc(Nc3ccc4c(c3)nnn4C(C)C)nc2n1-c1cccc(OC2CCN(C)CC2)n1] |

25. The compound of claim 1, wherein the compound is any one of the following compounds:

| # | Structure |
|---|---|
| 210 | 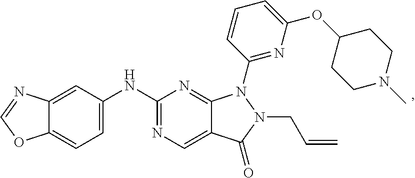 [SMILES: C=CCn1c(=O)c2cnc(Nc3ccc4ocnc4c3)nc2n1-c1cccc(OC2CCN(C)CC2)n1] |
| 213 | 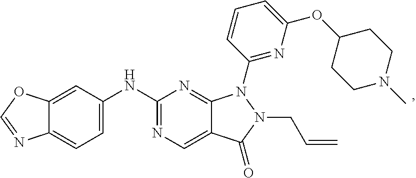 [SMILES: C=CCn1c(=O)c2cnc(Nc3ccc4ncoc4c3)nc2n1-c1cccc(OC2CCN(C)CC2)n1] |
| 215 | 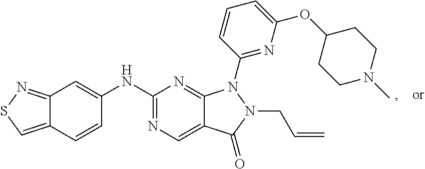 [SMILES: C=CCn1c(=O)c2cnc(Nc3ccc4csnc4c3)nc2n1-c1cccc(OC2CCN(C)CC2)n1] |
| 216 | 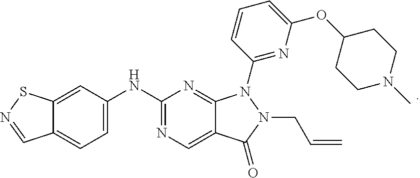 [SMILES: C=CCn1c(=O)c2cnc(Nc3ccc4cnsc4c3)nc2n1-c1cccc(OC2CCN(C)CC2)n1] |

26. A pharmaceutical composition comprising an effective amount of a compound of any one of claims 1-25; and a pharmaceutically acceptable carrier.

27. A method of inhibiting Wee1A kinase activity in a subject comprising the step of administering to the subject an effective amount of a compound of any one of claims 1-25, or a composition of claim 26.

28. A method of treating a subject suffering from a cancer or other disordered cell growth characterized by aberrant Wee1A kinase activity comprising the step of administering to the subject an effective amount of a compound of any one of claims 1-25, or a composition of claim 26.

29. The method of claim 28, wherein the subject is suffering from a cancer associated with inactivation of p53.

30. A method of inhibiting both Wee1A kinase and Myt1 kinase activity in a subject comprising the step of administering to the subject an effective amount of a compound of any one of claims 1-25 that is a dual inhibitor, or a composition of claim 26 comprising a dual inhibitor.

31. A method of treating a subject suffering from a cancer or other disordered cell growth characterized by both aberrant Wee1A kinase activity and aberrant Myt1 kinase activity comprising the step of administering to the subject an effective amount of a compound of any one of claims 1-25, or a composition of claim 26.

32. The method of any one of claim 28, 29, or 31, wherein the cancer is selected from a brain cancer, a cervicocerebral cancer, a cardiac cancer, a gastrointestinal cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

33. The method of claim 32, wherein the subject is suffering from a cancer selected from uterine serous carcinoma or a renal cancer.
